(19) United States
(12) Patent Application Publication
(10) Pub. No.: US 2022/0213061 A9
(43) Pub. Date: Jul. 7, 2022

(54) HETEROCYCLIC COMPOUNDS FOR MODULATING NR2F6

(71) Applicant: TES Pharma S.r.l., Corciano (IT)

(72) Inventor: Roberto PELLICCIARI, Perugia (IT)

(21) Appl. No.: 17/184,431

(22) Filed: Feb. 24, 2021

Related U.S. Application Data
(60) Provisional application No. 62/981,418, filed on Feb. 25, 2020.
(60) Provisional application No. 63/139,262, filed on Jan. 19, 2021.

(51) Int. Cl. C07D 401/12; C07D 417/14; C07D 513/04; C07D 207/16; C07D 409/04; C07D 495/04; C07D 409/14; C07D 403/12; C07D 401/14; C07D 405/14; C07D 405/12; A61P 35/00

(52) U.S. Cl. CPC C07D 401/12; C07D 417/14; C07D 513/04; C07D 207/16; C07D 409/04; A61P 35/00; C07D 409/14; C07D 403/12; C07D 401/14; C07D 405/14; C07D 405/12; C07D 495/04

(57) ABSTRACT

The present disclosure relates to compounds capable of modulating the activity of NR2F6. The compounds of the disclosure may be used in methods for the prevention and/or the treatment of diseases and disorders associated with modulating NR2F6 activity.

### CROSS REFERENCE TO RELATED APPLICATIONS

This application claims the benefit of U.S. Provisional Application No. 62/981,418, filed Feb. 25, 2020 and of U.S. Provisional Application No. 63/139,262, filed Jan. 19, 2021, the contents of which are incorporated herein by reference in their entireties.

### FIELD OF THE DISCLOSURE

The present disclosure relates to compounds capable of modulating the activity of NR2F6. The compounds of the disclosure may be used in methods for the prevention and/or the treatment of diseases and disorders associated with modulating NR2F6 activity.

### BACKGROUND OF THE DISCLOSURE

Nuclear receptor subfamily 2, group F, member 6 (NR2F6), also known as nuclear receptor Ear2 and COUP-TFIII, is an orphan member of the nuclear receptor (NR) superfamily of ligand-activated receptors. NRs exhibit a common modular structure and play important roles in homeostatic functions. Dysregulation of NR function has been linked to several pathological states (including; cancer, inflammatory, and metabolic syndromes).

NR2F6 modulates target gene expression through different mechanisms and competes with other NRs such as RAR for heterodimerization with RXR. Similar mechanism has been reported for thyroid hormone nuclear receptor (TR), whereas a direct interaction between NR2F6 and TR leads to reduced basal and T3-dependent activation of TR activity. NR2F6 activity plays an important role as a transrepressor through direct binding with other NRs.

NR2F6 limits immune system activation by repressing expression of pro-inflammatory cytokines such as IL-2, TNFα, IFNγ, and IL-17. Their downregulation is mediated by direct interaction between NR2F6 and nuclear factor of activated T cells (NFAT)/AP-1. NR2F6 and NFAT compete for the same loci. Moreover, the NR interacts with NFAT, preventing it to bind DNA response element. NR2F6 competes also with RORγ (NR1F3) for the same locus (i.e. IL-17a). Mutagenesis studies have demonstrated that NR2F6 transrepressor activity depends on the integrity of both its DNA- and ligand-binding domain. Post-translational modifications (i.e. phosphorylation) modulate NR2F6 functions.

Immunotherapy exploits small molecule compounds, monoclonal antibodies, cellular therapies, and pharmaceutical compositions thereof to modulate both adoptive and innate immune system. Immunotherapy has been successfully applied in different therapeutic fields such as oncology and autoimmune disorders.

NR2F6 plays a crucial role in immune-mediated cancer surveillance. NR2F6 deficient mice display an immune contexture favoring antitumor responses, for example through the upregulation of IL-17 and other pro-inflammatory cytokines (TNFα, IFNγ, and IL-2) in both CD4+ and CD8+. Therefore, NR2F6 controls the amplitude of tumor immunity and acts as a novel potential immune checkpoint for anticancer therapy.

NR2F6 cross-talks with other immune checkpoints. For instance, NR2F6 genetic ablation shows an increased expression of PD-L1 in immune cells. Moreover, both germinal NR2F6 knockout as well as adoptive cell therapy (ACT) which embodies acute NR2F6 knockout show synergic anticancer effects in combination with blockade of other immune checkpoints (i.e. PD-L1, CTLA-4). Both NR2F6 inhibition and downregulation can increase efficacy of immune checkpoint inhibitors.

Genomic studies raise NR2F6 as a pivotal protein that regulates cell differentiation. NR2F6 plays a crucial role in maintaining the clonogenic status within the leukemia cell hierarchy. Moreover, NR2F6 is overexpressed in undifferentiated cancer stem cells, while its ablation led to differentiation and consequent increasing of apoptosis rate.

NR2F6 KO mice are hypersusceptible to inflammatory states (i.e. experimental autoimmune encephalomyelitis (EAE)) and they demonstrate both a faster onset and an overall higher clinical score than wild-type mice. NR2F6 KO mice are also characterized by higher numbers of CNS-infiltrating IL-17-IFNγ double-positive CD4+ effector T cells and hyperreactive Th17 cells.

Besides controlling immunity and inflammation, NR2F6 activity is crucial for intestinal homeostasis. NR2F6 transactivates genes responsible for the maintenance of gut barrier such as Muc2. Genetic ablation of NR2F6 worsens conditions in colitis mouse model compared to wild type mice and Nr2f6−/− mice show increased susceptibility to DSS-induced colitis compared with wild-type mice, characterized by an aggravated clinical disease phenotype and enhanced immune cell infiltration. Nr2f6−/−CD4+ T cells are not the primary cause of increased colonic inflammation and disease pathology. Rather, loss of NR2F6 in colon epithelial cells enhanced intestinal permeability, leading to spontaneous colitis in Nr2f6-deficient mice. NR2F6 directly transactivates Muc2 expression via in human colon carcinoma cell line LoVo and primary mouse colon epithelial cells. Loss of NR2F6 alters intestinal permeability and results in spontaneous late-onset colitis in Nr2f6-deficient mice. Selective agonists of NR2F6 might represent a novel therapeutic strategy in the treatment of certain forms of human IBD.

NR2F6 modulation thus represents a novel approach to regulate adoptive and innate immunity in several diseases (including cancer) and immune-related disorders (such as autoimmune diseases), and to increase efficacy towards immune checkpoint inhibitors and adoptive cell therapy. Moreover, NR2F6 modulation also gastrointestinal disorders. The present disclosure is directed to, in certain embodiments, methods of using small molecule compounds capable of modulating NR2F6 activity and pharmaceutical compositions thereof, as well as to methods of making the compounds and pharmaceutical compositions thereof.

### SUMMARY OF THE DISCLOSURE

The present disclosure provides a compound represented by Formula (I-A) or (II-A):

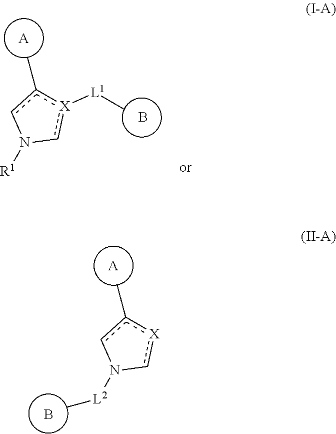

and pharmaceutically acceptable salts and tautomers thereof, wherein:

each [symbol] independently represents a single bond or a double bond;

X is N, NH, C, CH, or CH₂;

R¹ is H, C₁₋₆alkyl, cycloalkyl, heterocyclyl, —C(O)R^{1a}, —CH₂-aryl, —CH₂-heteroaryl, aryl, or heteroaryl; wherein R^{1a} is C₁₋₆alkyl; and wherein —CH₂-aryl, —CH₂-heteroaryl, aryl, and heteroaryl are optionally substituted with C₁₋₆alkyl or halo;

A is alkyl, cycloalkyl, heterocyclyl, a fused bicyclic aryl, a fused bicyclic heteroaryl, —CH₂-aryl, —CH₂-heteroaryl, aryl, or heteroaryl; wherein the aryl or heteroaryl is optionally substituted with aryl, heteroaryl, —Y^{A}-aryl, or —Y^{A}-heteroaryl; wherein Y^{A} is —O—, —C(O)—, —N(R^{A1})—, S(O)—, or —S(O)₂—; wherein R^{A1} is H or C₁₋₆alkyl;
wherein the fused bicyclic aryl, the fused bicyclic heteroaryl, —CH₂-aryl, —CH₂— heteroaryl, each aryl, and each heteroaryl are optionally substituted with one or more substituents selected from the group consisting of alkyl, halo, haloalkyl, —CN, —N(R^{A})₂, —OH, and —O-alkyl; wherein each R^{A} is independently H or C₁₋₆alkyl;

L¹ is —C(O)—NR^{L1}—, —O—C(S)—NR^{L1}—, —O—C(O)—NR^{L1}—, —NR^{L1}—C(O)—, —NR^{L1}—C(O)—O—, —NH—C(O)—NH—, —NR^{L1}—C(S)—NR^{L1}—, —NR^{L1}—S(O)₂—, —S(O)₂—NR^{L1}—, —CH₂—CH₂—, —CH₂—NR^{L1}—, —NR^{L1}—CH₂—, —CH₂—O—, —O—CH₂—, —O—, —NH—, —C(O)-azetidinyl, —CH₂—NR^{L1}—C(O)—, —C(O)—NR^{L1}—CH₂—, or —C(O)—; wherein each R^{L1} is independently H or C₁₋₆alkyl; and

L² is —C(O)—NR^{L2}—, —S(O)₂—NR^{L2}—, —CH₂—CH₂—, —C(S)—NR^{L2}—, —C(O)—, or —S(O)₂—; wherein each R^{L2} is independently H or C₁₋₆alkyl; and

B is a fused bicyclic aryl, a fused bicyclic heteroaryl, —CH₂-aryl, —CH₂-heteroaryl, aryl, heteroaryl, cycloalkyl, —CH₂-heterocyclyl, or heterocyclyl, wherein the aryl, heteroaryl, cycloalkyl, or heterocyclyl is optionally substituted with aryl, heteroaryl, —Y^{B}-aryl, —Y^{B}— heteroaryl, —Y^{B}-heterocyclyl, or cycloalkyl; wherein Y^{B} is —O—, —CH₂—, —C(O)—, —N(R^{B1})—, —S(O)—, or —S(O)₂—; wherein R^{B1} is H or C₁₋₆alkyl;
wherein the fused bicyclic aryl, the fused bicyclic heteroaryl, —CH₂-aryl, —CH₂— heteroaryl, each aryl, each heteroaryl, each cycloalkyl, —CH₂-heterocyclyl, and each heterocyclyl are optionally substituted with one or more substituents selected from the group consisting of alkyl, halo, haloalkyl, —CN, —N(R^{B2})₂, —OH, —O-alkyl, and oxo;

wherein each R^{B2} is independently H or C₁₋₆alkyl;

wherein when the compound is Formula (I-A); A is optionally substituted phenyl or thiophenyl, and L¹ is —C(O)—NH—; then B is not

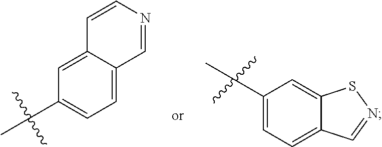 [SMILES: *c1ccc2cnccc2c1 ; *c1ccc2cnsc2c1]

wherein when the compound is Formula (I-A); A is phenyl, and L¹ is —C(O)—NH—; then B is not

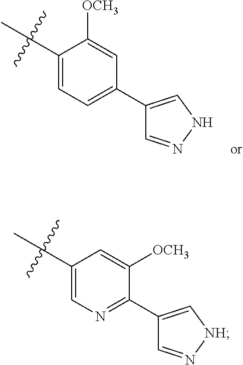 [SMILES: *c1ccc(-c2cn[nH]c2)cc1OC ; "or" ; *c1cnc(-c2cn[nH]c2)c(OC)c1]

wherein when the compound is Formula (I-A); A is a substituted phenyl and B is a substituted phenyl, then L¹ is not —C(O)—NH—, —NH—C(O)—, —NCH₃—C(O)—, or —NH—C(O)—NH—;

wherein when the compound is Formula (I-A); L¹ is —C(O)—NR^{L1}—CH₂— and B is an optionally substituted phenyl, substituted pyridyl, or

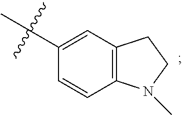 [SMILES: *c1ccc2c(c1)CCN2C]

then A is not substituted phenyl, substituted pyridyl, substituted thiophenyl, substituted thiazolyl, substituted pyrazolyl

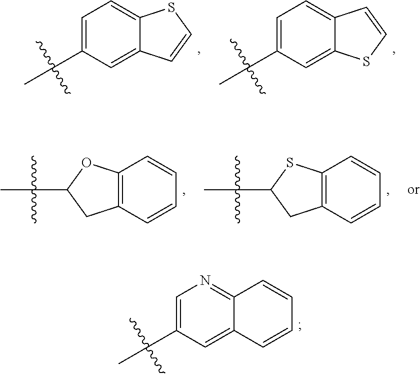 [SMILES: *c1ccc2sccc2c1 ; *c1ccc2ccsc2c1 ; *C1Cc2ccccc2O1 ; *C1Cc2ccccc2S1 ; *c1cnc2ccccc2c1]

wherein when the compound is Formula (I-A); B is optionally substituted —CH₂-aryl and A is optionally substituted aryl; then L¹ is not —C(O)—NH—;

wherein when the compound is Formula (II-A); A is optionally substituted phenyl and B is optionally substituted phenyl, then L¹ is not —C(O)—NCH₃—.

The present disclosure provides a compound represented by Formula (I) or (II):

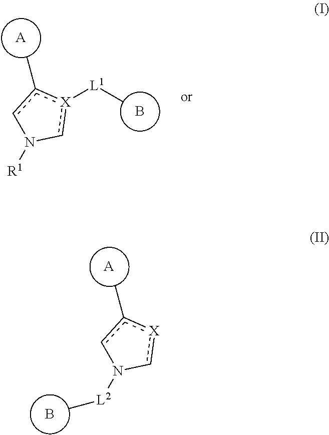

and pharmaceutically acceptable salts and tautomers thereof, wherein:

each [symbol] independently represents a single bond or a double bond;

X is N, NH, C, CH, or CH₂;

R¹ is H, C₁₋₆alkyl, cycloalkyl, heterocyclyl, —C(O)R^{1a}, —CH₂-aryl, —CH₂-heteroaryl, aryl, or heteroaryl; wherein R^{1a} is C₁₋₆alkyl; and wherein —CH₂-aryl, —CH₂-heteroaryl, aryl, and heteroaryl are optionally substituted with C₁₋₆alkyl or halo;

A is alkyl, cycloalkyl, heterocyclyl, a fused bicyclic aryl, a fused bicyclic heteroaryl, —CH₂-aryl, —CH₂-heteroaryl, aryl, or heteroaryl; wherein the aryl or heteroaryl is optionally substituted with aryl, heteroaryl, —Y^{A}-aryl, or —Y^{A}-heteroaryl; wherein Y^{A} is —O—, —C(O)—, —N(R^{A1})—, —S(O)—, or —S(O)₂—; wherein R^{A1} is H or C₁₋₆alkyl;
wherein the fused bicyclic aryl, the fused bicyclic heteroaryl, —CH₂-aryl, —CH₂— heteroaryl, each aryl, and each heteroaryl are optionally substituted with one or more substituents selected from the group consisting of alkyl, halo, —CN, —N(R^{A})₂, —OH, and —O-alkyl; wherein each R^{A} is independently H or C₁₋₆alkyl;

L¹ is —C(O)—NR^{L1}—, —O—C(S)—NR^{L1}—, —O—C(O)—NR^{L1}—, —NR^{L1}—C(O)—, —NR^{L1}—C(O)—O—, —NH—C(O)—NH—, —NR^{L1}—C(S)—NR^{L1}—, —NR^{L1}—S(O)₂—, —S(O)₂—NR^{L1}—, —CH₂—CH₂—, —CH₂—NR^{L1}—, —NR^{L1}—CH₂—, —CH₂—O—, —O—CH₂—, —O—, —NH—, —C(O)-azetidinyl, —CH₂—NR^{L1}—C(O)—, or —C(O)—NR^{L1}—CH₂—; wherein each R^{L1} is independently H or C₁₋₆alkyl; and

L² is —C(O)—NR^{L2}—, —S(O)₂—NR^{L2}—, —CH₂—CH₂—, —C(S)—NR^{L2}—, —C(O)—, or —S(O)₂—; wherein each R^{L2} is independently H or C₁₋₆alkyl; and

B is a fused bicyclic aryl, a fused bicyclic heteroaryl, —CH₂-aryl, —CH₂-heteroaryl, aryl, heteroaryl, cycloalkyl, or —CH₂-heterocyclyl, wherein the aryl or heteroaryl is optionally substituted with aryl, heteroaryl, —Y^{B}-aryl, or —Y^{B}-heteroaryl; wherein Y^{B} is —O—, —C(O)—, —N(R^{B1})—, —S(O)—, or —S(O)₂—; wherein R^{B1} is H or C₁₋₆alkyl;
wherein the fused bicyclic aryl, the fused bicyclic heteroaryl, —CH₂-aryl, —CH₂— heteroaryl, each aryl, each heteroaryl, cycloalkyl, and —CH₂-heterocyclyl are optionally substituted with one or more substituents selected from the group consisting of alkyl, halo, —CN, —N(R^{B2})₂, —OH, and —O-alkyl; wherein each R^{B2} is independently H or C₁₋₆alkyl;

wherein when the compound is Formula (I); A is optionally substituted phenyl or thiophenyl, and L¹ is —C(O)—NH—; then B is not

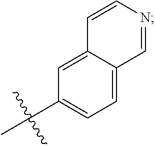 [SMILES: *c1ccc2cnccc2c1]

wherein when the compound is Formula (I); A is a substituted phenyl and B is a substituted phenyl, then L¹ is not —C(O)—NH—, —NH—C(O)—, —NCH₃—C(O)—, or —NH—C(O)—NH—;

wherein when the compound is Formula (I); B is optionally substituted —CH₂-aryl and A is optionally substituted aryl; then L¹ is not —C(O)—NH—;

wherein when the compound is Formula (II); A is optionally substituted phenyl and B is optionally substituted phenyl, then L¹ is not —C(O)—NCH₃—.

The present disclosure provides a compound represented by Formula (III):

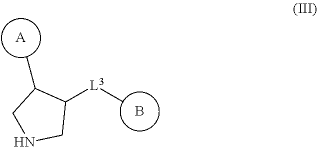

and pharmaceutically acceptable salts and tautomers thereof, wherein:

A is aryl or 5- to 6-membered heteroaryl, wherein the aryl and heteroaryl are optionally substituted with one or more substituents selected from the group consisting of alkyl, halo, —OH, and —O-alkyl;

L³ is —C(O)—NR^{L3}—, —O—C(S)—NR^{L3}—, —O—C(O)—NR^{L3}—, —NR^{L3}—C(O)—, —NR^{L3}—C(S)—NR^{L3}—, —NR^{L3}—S(O)₂—, —S(O)₂—NR^{L3}—, —CH₂—CH₂—, —CH₂—NR^{L3}—, —NR^{L3}—CH₂—, —CH₂—O—, —O—CH₂—, or —O—; wherein each R^{L3} is independently hydrogen or C₁₋₆alkyl; and

B is a fused bicyclic aryl, a fused bicyclic heteroaryl, —CH₂-aryl, —CH₂-heteroaryl, aryl, or heteroaryl, wherein the aryl or heteroaryl is optionally substituted with aryl or heteroaryl;
wherein the fused bicyclic aryl, the fused bicyclic heteroaryl, —CH₂-aryl, —CH₂— heteroaryl, each aryl, and each heteroaryl are optionally substituted with one or more substituents selected from the group consisting of alkyl, halo, —OH, and —O-alkyl;

wherein when A is optionally substituted phenyl or thiophenyl, and L³ is —C(O)—NH—; then B is not

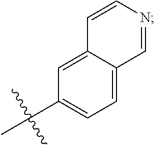 [SMILES: *c1ccc2cnccc2c1]

wherein when A is a substituted phenyl and B is a substituted phenyl, then L³ is not —C(O)—NH—, —NH—C(O)—, —NCH₃—C(O)—, or —NH—C(O)—NH—;

wherein when B is optionally substituted —CH₂-aryl and A is optionally substituted aryl; then L³ is not —C(O)—NH—.

The present disclosure provides a pharmaceutical composition comprising a compound of Formula (I-A), (II-A), (I), (II), or (III), or a pharmaceutically acceptable salt or tautomer thereof, and a pharmaceutically acceptable excipient.

The present disclosure provides a compound of Formula (I-A), (II-A), (I), (II), or (III), or a pharmaceutically acceptable salt thereof, for use as a medicament. Another aspect of the present disclosure provides a pharmaceutical composition comprising a compound of Formula (I-A), (II-A), (I), (II), or (III), or a pharmaceutically acceptable salt thereof, for use as a medicament.

The present disclosure provides a method of modulating activity of NR2F6 by exposure of NR2F6 to an effective amount of a compound of Formula (I-A), (II-A), (I), (II), or (III), or a pharmaceutically acceptable salt or tautomer thereof, or a pharmaceutical composition comprising a compound of Formula (I-A), (II-A), (I), (II), or (III), or a pharmaceutically acceptable salt or tautomer thereof. The present disclosure provides a method of treating or reducing the effect of a disease or disorder associated with NR2F6 modulation, the method comprising administration of an effective amount of a compound of Formula (I-A), (II-A), (I), (II), or (III), or a pharmaceutically acceptable salt or tautomer thereof, or the pharmaceutical composition comprising a compound of Formula (I-A), (II-A), (I), (II), or (III), or a pharmaceutically acceptable salt or tautomer thereof.

The present disclosure provides a compound of Formula (I-A), (II-A), (I), (II), or (III), or a pharmaceutically acceptable salt or tautomer thereof, or a pharmaceutical composition comprising a compound of Formula (I-A), (II-A), (I), (II), or (III), or a pharmaceutically acceptable salt or tautomer thereof for use in modulating activity of NR2F6 by exposure of NR2F6. The present disclosure provides a compound of Formula (I-A), (II-A), (I), (II), or (III), or a pharmaceutically acceptable salt or tautomer thereof, or a pharmaceutical composition comprising a compound of Formula (I-A), (II-A), (I), (II), or (III), or a pharmaceutically acceptable salt or tautomer thereof for use in treating or reducing the effect of a disease or disorder associated with NR2F6 modulation.

The present disclosure provides use of a compound of Formula (I-A), (II-A), (I), (II), or (III), or a pharmaceutically acceptable salt or tautomer thereof, or a pharmaceutical composition comprising a compound of Formula (I-A), (II-A), (I), (II), or (III), or a pharmaceutically acceptable salt or tautomer thereof for modulating activity of NR2F6 by exposure of NR2F6. The present disclosure provides use of a compound of Formula (I-A), (II-A), (I), (II), or (III), or a pharmaceutically acceptable salt or tautomer thereof, or a pharmaceutical composition comprising a compound of Formula (I-A), (II-A), (I), (II), or (III), or a pharmaceutically acceptable salt or tautomer thereof for treating or reducing the effect of a disease or disorder associated with NR2F6 modulation.

The present disclosure provides use of a compound of Formula (I-A), (II-A), (I), (II), or (III), or a pharmaceutically acceptable salt or tautomer thereof, or a pharmaceutical composition comprising a compound of Formula (I-A), (II-A), (I), (II), or (III), or a pharmaceutically acceptable salt or tautomer thereof, in the manufacture of a medicament for modulating activity of NR2F6. The present disclosure provides use of a compound of Formula (I-A), (II-A), (I), (II), or (III), or a pharmaceutically acceptable salt or tautomer thereof, or a pharmaceutical composition comprising a compound of Formula (I-A), (II-A), (I), (II), or (III), or a pharmaceutically acceptable salt or tautomer thereof, in the manufacture of a medicament for treating or reducing the effect of a disease or disorder associated with NR2F6 modulation.

Unless otherwise defined, all technical and scientific terms used herein have the same meaning as commonly understood by one of ordinary skill in the art to which this disclosure belongs. In the specification, the singular forms also include the plural unless the context clearly dictates otherwise. Although methods and materials similar to or equivalent to those described herein can be used in the practice and testing of the disclosure, suitable methods and materials are described below. All publications, patent applications, patents, and other references mentioned herein are incorporated by reference. The references cited herein are not admitted to be prior art to the claimed disclosure. In the case of conflict, the present specification, including definitions, will control. In addition, the materials, methods, and examples are illustrative only and not intended to be limiting.

Other features and advantages of the disclosure will be apparent from the following detailed description and claims.

### DETAILED DESCRIPTION OF THE DISCLOSURE

All references, including any patent or patent application, cited in this specification are hereby incorporated by reference. No admission is made that any reference constitutes prior art. Further, no admission is made that any of the prior art constitutes part of the common general knowledge in the art.

As used throughout this disclosure, the following terms, unless otherwise indicated, shall be understood to have the following meanings. If a term is missing, the conventional term as known to one skilled in the art controls.

As used herein, the terms “including,” “containing,” and “comprising” are used in their open, non-limiting sense. Throughout the description and claims of this specification, the words “comprise” and “contain” and variations of the words, for example “comprising” and “comprises”, mean “including but not limited to” and do not exclude other moieties, additives, components, integers, or steps. Throughout the description and claims of this specification, the singular encompasses the plural unless the context otherwise requires. In particular, where the indefinite article is used, the specification is to be understood as contemplating plurality as well as singularity, unless the context requires otherwise.

The articles “a” and “an” as used in this disclosure may refer to one or more than one (i.e., to at least one) of the grammatical object of the article. By way of example, “an element” may mean one element or more than one element.

The term “and/or” as used in this disclosure may mean either “and” or “or” unless indicated otherwise.

To provide a more concise description, some of the quantitative expressions given herein are not qualified with the term “about.” It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value. Whenever a yield is given as a percentage, such yield refers to a mass of the entity for which the yield is given with respect to the maximum amount of the same entity that could be obtained under the particular stoichiometric conditions. Concentrations that are given as percentages refer to mass ratios, unless indicated differently.

The term “alkyl” as used herein refers to a saturated, straight, or branched hydrocarbon chain. The hydrocarbon chain preferably contains from one to eight carbon atoms (C₁₋₈-alkyl), such as from one to six carbon atoms (C₁₋₆-alkyl), such as from one to four carbon atoms (C₁₋₄-alkyl), including methyl, ethyl, propyl, isopropyl, butyl, isobutyl, secondary butyl, tertiary butyl, pentyl, isopentyl, neopentyl, tertiary pentyl, hexyl, isohexyl, heptyl and octyl. In a certain embodiment, “alkyl” represents a C₁₋₄-alkyl group, which may in particular include methyl, ethyl, propyl, isopropyl, butyl, isobutyl, secondary butyl, and tertiary butyl. Correspondingly, the term “alkylene” means the corresponding biradical (-alkyl-).

The term “cycloalkyl” or “carbocycle” as used herein refers to a cyclic alkyl group, preferably containing from three to ten carbon atoms (C₃₋₁₀-cycloalkyl or C₃₋₁₀-carbocycle), such as from three to eight carbon atoms (C₃₋₈-cycloalkyl or C₃₋₁₀-carbocycle), preferably from three to six carbon atoms (C₃₋₆-cycloalkyl or C₃₋₁₀-carbocycle), including cyclopropyl, cyclobutyl, cyclopentyl, cyclohexyl, cycloheptyl, and cyclooctyl. Furthermore, the term “cycloalkyl” as used herein may also include polycyclic groups such as for example bicyclo[2.2.2]octyl, bicyclo[2.2.1]heptanyl, decalinyl, and adamantyl. Correspondingly, the term “cycloalkylene” means the corresponding biradical (-cycloalkyl-). “Cycloalkyl” includes ring systems where the cycloalkyl ring, as defined above, is fused with one or more cycloalkyl, heterocyclyl, aryl, or heteroaryl groups, wherein the point of attachment is on a cycloalkyl ring. Alkyl and cycloalkyl groups may be optionally substituted with 1-4 substituents. Examples of substituents on alkyl groups include, but are not limited to, alkyl, alkenyl, alkynyl, halogen, haloalkyl, alkoxy, heteroaryl, aryl, carbocyclyl, hydroxyl, carbamoyl, oxo, and —CN.

The term “alkenyl” as used herein refers to a straight or branched hydrocarbon chain or cyclic hydrocarbons containing one or more double bonds, including di-enes, tri-enes and poly-enes. Typically, the alkenyl group comprises from two to eight carbon atoms (C₂₋₈-alkenyl), such as from two to six carbon atoms (C₂₋₆-alkenyl), in particular from two to four carbon atoms (C₂₋₄-alkenyl), including at least one double bond. Examples of alkenyl groups include ethenyl; 1- or 2-propenyl; 1-, 2- or 3-butenyl, or 1,3-but-dienyl; 1-, 2-, 3-, 4- or 5-hexenyl, or 1,3-hex-dienyl, or 1,3,5-hex-trienyl; 1-, 2-, 3-, 4-, 5-, 6-, or 7-octenyl, or 1,3-octadienyl, or 1,3,5-octatrienyl, or 1,3,5,7-octatetraenyl, or cyclohexenyl. Correspondingly, the term “alkenylene” means the corresponding biradical (-alkenyl-). Alkenyl groups may be optionally substituted with 1-4 substituents. Examples of substituents on alkenyl groups include, but are not limited to, alkyl, alkenyl, alkynyl, halogen, haloalkyl, alkoxy, heteroaryl, aryl, carbocyclyl, hydroxyl, carbamoyl, oxo, and —CN.

The term “alkynyl” as used herein refers to a straight or branched hydrocarbon chain containing one or more triple bonds, including di-ynes, tri-ynes, and poly-ynes. Typically, the alkynyl group comprises of from two to eight carbon atoms (C₂₋₈-alkynyl), such as from two to six carbon atoms (C₂₋₆-alkynyl), in particular from two to four carbon atoms (C₂₋₄-alkynyl), including at least one triple bond. Examples of certain alkynyl groups include ethynyl; 1- or 2-propynyl; 1-, 2- or 3-butynyl, or 1,3-but-diynyl; 1-, 2-, 3-, 4- or 5-hexynyl, or 1,3-hex-diynyl, or 1,3,5-hex-triynyl; 1-, 2-, 3-, 4-, 5-, 6-, or 7-octynyl, or 1,3-oct-diynyl, or 1,3,5-oct-triynyl, or 1,3,5,7-oct-tetraynyl. Correspondingly, the term “alkynylene” means the corresponding biradical (-alkynyl-). Alkynyl groups may be optionally substituted with 1-4 substituents. Examples of substituents on alkynyl groups include, but are not limited to, alkyl, alkenyl, alkynyl, halogen, haloalkyl, alkoxy, heteroaryl, aryl, carbocyclyl, hydroxyl, carbamoyl, oxo, and —CN.

The terms “halo” and “halogen” as used herein refer to fluoro, chloro, bromo or iodo. Thus, a trihalomethyl group represents, e.g., a trifluoromethyl group, or a trichloromethyl group. Preferably, the terms “halo” and “halogen” designate fluoro or chloro.

The term “haloalkyl” as used herein refers to an alkyl group, as defined herein, which is substituted one or more times with one or more halogen. Examples of haloalkyl groups include, but are not limited to, trifluoromethyl, difluoromethyl, pentafluoroethyl, trichloromethyl, etc.

The term “alkoxy” as used herein refers to an “alkyl-O—” group, wherein alkyl is as defined above.

The term “oxo” as used herein refers to an “═O” group.

The term “amine” as used herein refers to primary (R—NH₂, R≠H), secondary ((R)₂—NH, (R)₂≈H), and tertiary ((R)₃—N, R≠H) amines. A substituted amine is intended to mean an amine where at least one of the hydrogen atoms has been replaced by the substituent.

The term “carbamoyl” as used herein refers to a “H₂N(C═O)—” group.

The term “aryl”, as used herein, refers to a monocyclic or polycyclic group having at least one hydrocarbon aromatic ring, wherein all of the ring atoms of the at least one hydrocarbon aromatic ring are carbon. Wherein aryl includes a polycyclic system, no aromatic ring heteroatoms are present. Aryl may include groups with a single aromatic ring (e.g., phenyl) and multiple fused aromatic rings (e.g., naphthyl, anthryl). Aryl may further include groups with one or more aromatic hydrocarbon rings fused to one or more non-aromatic hydrocarbon rings (e.g., fluorenyl; 2,3-dihydro-1H-indene; 1,2,3,4-tetrahydronaphthalene). In certain embodiments, aryl includes groups with an aromatic hydrocarbon ring fused to a non-aromatic ring, wherein the non-aromatic ring comprises at least one ring hetero atom independently selected from the group consisting of N, O, and S. For example, in some embodiments, aryl includes groups with a phenyl ring fused to a non-aromatic ring, wherein the non-aromatic ring comprises at least one ring hetero atom independently selected from the group consisting of N, O, and S (e.g., chromane; thiochromane; 2,3-dihydrobenzofuran; indoline). In some embodiments, aryl as used herein has from 6 to 14 carbon atoms ((C₆-C₁₄)aryl), or 6 to 10 carbon atoms ((C₆-C₁₀)aryl). Where the aryl includes fused rings, the aryl may connect to one or more substituents or moieties of the formulae described herein through any atom of the fused ring for which valency permits.

Examples of certain aryl moieties include phenyl, naphthyl, indenyl, indanyl, fluorenyl, biphenyl, indenyl, naphthyl, anthracenyl, phenanthrenyl, pentalenyl, azulenyl, and biphenylenyl. Examples of certain “aryls” include phenyl, naphthyl, and indanyl, such as phenyl, unless otherwise stated. Any aryl used may be optionally substituted. Correspondingly, the term “arylene” means the corresponding biradical (-aryl-). Aryl groups may be optionally substituted with 1-4 substituents. Examples of substituents on aryl groups include, but are not limited to, alkyl, alkenyl, alkynyl, halogen, haloalkyl, alkoxy, heteroaryl, aryl, carbocyclyl, hydroxyl, and —CN.

Fused bicyclic aryl refers to a polycyclic group with two fused rings having at least one hydrocarbon aromatic ring, wherein all of the ring atoms of the at least one hydrocarbon aromatic ring are carbon. In certain embodiments, fused bicyclic aryl comprises two aromatic rings.

As noted above, aryl may further include groups with one or more aromatic hydrocarbon rings fused to one or more non-aromatic hydrocarbon rings (e.g., fluorenyl; 2,3-dihydro-1H-indene; 1,2,3,4-tetrahydronaphthalene). In certain embodiments, aryl includes groups with an aromatic hydrocarbon ring fused to a non-aromatic ring, wherein the non-aromatic ring comprises at least one ring hetero atom independently selected from the group consisting of N, O, and S. For example, in some embodiments, aryl includes groups with a phenyl ring fused to a non-aromatic ring, wherein the non-aromatic ring comprises at least one ring hetero atom independently selected from the group consisting of N, O, and S (e.g., chromane; thiochromane; 2,3-dihydrobenzofuran; indoline; 2,3-dihydrobenzo[b][1,4]dioxine). In certain embodiments, fused bicyclic aryl comprises an aromatic ring and a non-aromatic ring.

The term “heteroaryl”, as used herein, refers to a monocyclic or polycyclic group comprising at least one aromatic ring, wherein the aromatic ring comprises at least one ring heteroatom independently selected from the group consisting of N, O, and S. The heteroaryl group may comprise 5, 6, 7, 8, 9, 10, 11, 12, or more ring atoms, where ring atoms refer to the sum of carbon and heteroatoms in the one or more rings (e.g., be a 5-membered, 6-membered, 7-membered, 8-membered, 9-membered, 10-membered, 11-membered, or 12-membered heteroaryl). In some embodiments, heteroaryl includes groups with an aromatic ring that comprises at least one ring heteroatom independently selected from the group consisting of N, O, and S, (e.g., pyridinyl, pyrazinyl, furanyl, thiophenyl). In certain embodiments, heteroaryl includes polycyclic groups with an aromatic ring comprising at least one ring heteroatom, fused to anon-aromatic hydrocarbon ring (e.g., 5,6,7,8-tetrahydroquinolinyl; 4,5,6,7-tetrahydroisobenzofuranyl). In some embodiments, heteroaryl includes polycyclic groups with an aromatic ring comprising at least one ring heteroatom fused to an aromatic hydrocarbon ring (e.g., quinolinyl, quinoxalinyl, benzothiazolyl). In still further embodiments, heteroaryl includes polycyclic groups with two fused aromatic rings, wherein each ring comprises at least one ring heteroatom (e.g., naphthyridinyl). Heteroaryl may include groups comprising 1 to 5 ring heteroatoms, 1 to 4 ring heteroatoms, 1 to 3 ring heteroatoms, 1 or 2 ring heteroatoms, or 1 ring heteroatom, wherein each ring heteroatom is independently selected from the group consisting of N, O, and S. In one example, a heteroaryl has 3 to 8 ring carbon atoms, with 1 to 3 ring heteroatoms independently selected from N, O, and S. Examples of heteroaryl groups include pyridyl, pyridazinyl, pyrimidinyl, benzothiazolyl, and pyrazolyl.

Examples of certain heteroaryl moieties include N-hydroxytetrazolyl, N-hydroxytriazolyl, N-hydroxyimidazolyl, furanyl, triazolyl, pyranyl, thiadiazinyl, benzothiophenyl, dihydro-benzo[b]thiophenyl, xanthenyl, isoindanyl, acridinyl, benzisoxazolyl, quinolinyl, isoquinolinyl, phteridinyl, azepinyl, diazepinyl, imidazolyl, thiazolyl, carbazolyl, pyridinyl, pyridazinyl, pyrimidinyl, pyrazolyl, pyrazinyl, tetrazolyl, furyl, thienyl, isoxazolyl, oxazolyl, isothiazolyl, pyrrolyl, indolyl, benzimidazolyl, benzofuranyl, cinnolinyl, indazolyl, indolizinyl, phthalazinyl, triazinyl, isoindolyl, purinyl, oxadiazolyl, thiadiazolyl, furazanyl, benzofurazanyl, benzothiophenyl, benzotriazolyl, benzothiazolyl, benzoxazolyl, quinazolinyl, quinoxalinyl, naphthyridinyl, dihydroquinolyl, tetrahydroquinolyl, dihydroisoquinolyl, tetrahydroisoquinolyl, benzofuryl, furopyridinyl, pyrolopyrimidinyl, azaindolyl, pyrazolinyl, 1,2,4-oxadiazol-5(4H)-one, and pyrazolidinyl. Non-limiting examples of partially hydrogenated derivatives are 1,2,3,4-tetrahydronaphthyl, 1,4-dihydronaphthyl, and 1-octalin. Correspondingly, the term “heteroarylene” means the corresponding biradical (-heteroaryl-). Heteroaryl groups may be optionally substituted with 1-4 substituents. Examples of substituents on heteroaryl groups include, but are not limited to, alkyl, alkenyl, alkynyl, halogen, haloalkyl, alkoxy, heteroaryl, aryl, carbocyclyl, hydroxyl, and —CN.

Fused bicyclic heteroaryl refers to a polycyclic group with two fused rings comprising at least one aromatic ring, wherein the aromatic ring comprises at least one ring heteroatom independently selected from the group consisting of N, O, and S. In certain embodiments, fused bicyclic heteroaryl comprises two aromatic rings.

The term “heterocyclyl” as used herein refers to a single saturated or partially unsaturated non-aromatic ring or a non-aromatic multiple ring system that has at least one heteroatom in the ring (at least one annular heteroatom selected from oxygen, nitrogen, and sulfur). Heterocyclyl” includes ring systems where the heterocyclyl ring, as defined above, is fused with one or more cycloalkyl, cycloalkenyl, heterocyclyl, aryl, or heteroaryl groups, wherein the point of attachment is on a heterocyclic ring, and, in such instances, the number of ring members recited continues to designate the number of annular atoms in the heterocyclic ring containing the point of attachment. Examples of heterocyclic groups include piperidinyl (6-membered heterocycle with 6 annular atoms), azepanyl (7-membered heterocycle with 7 annular atoms), and 3-chromanyl (6-membered heterocycle with 10 annular atoms)

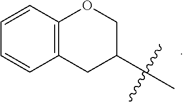 [SMILES: *C1COc2ccccc2C1]

Examples of heterocyclic groups are oxetane, pyrrolidinyl, pyrrolyl, 3H-pyrrolyl, oxolanyl, furanyl, thiolanyl, thiophenyl, pyrazolyl, pyrazolidinyl, imidazolyl, imidazolidinyl, 3H-pyrazolyl, 1,2-oxazolyl, 1,3-oxazolyl, 1,2-thiazolyl, 1,3-thiazolyl, 1,2,5-oxadiazolyl, piperidinyl, pyridinyl, oxanyl, 2-H-pyranyl, 4-H-pyranyl, thianyl, 2H-thiopyranyl, pyridazinyl, 1,2-diazinanyl, pyrimidinyl, 1,3-diazinanyl, pyrazinyl, piperazinyl, 1,4-dioxinyl, 1,4-dioxanyl, 1,3-diazinanyl, 1,4-oxazinyl, morpholino, thiomorpholino, 1,4-oxathianyl, benzofuranyl, isobenzofuranyl, indazolyl, benzimidazolyl, quinolinyl, isoquinolinyl, chromayl, isochromanyl, 4H-chromenyl, 1H-isochromenyl, cinnolinyl, quinazolinyl, quinoxalinyl, phthalazinyl, purinyl, naphthyridinyl, pteridinyl, indolizinyl, 1H-pyrrolizinyl, 4H-quinolizinyl, and aza-8-bicyclo[3.2.1]octane. Correspondingly, the term “heterocyclylene” means the corresponding biradical (-heterocyclyl-). Heterocyclyl groups may be optionally substituted with 1-4 substituents. Examples of substituents on heterocyclyl groups include, but are not limited, to alkyl, alkenyl, alkynyl, halogen, haloalkyl, alkoxy, heteroaryl, aryl, carbocyclyl, hydroxyl, and —CN.

In the present specification, the structural formula of the compound represents a certain isomer for convenience in some cases, but the present disclosure includes all isomers, such as geometrical isomers, optical isomers based on an asymmetrical carbon, stereoisomers, tautomers, and the like. Accordingly, it should be understood that the definition of compounds of Formula (I-A), (II-A), (I), (II), or (III) include each and every individual isomer corresponding to the Formula: Formula (I-A), (II-A), (I), (II), or (III), including cis-trans isomers, stereoisomers and tautomers, as well as racemic mixtures of these and pharmaceutically acceptable salts thereof. Hence, the definition of compounds of Formula (I-A), (II-A), (I), (II), or (III) are also intended to encompass all R- and S-isomers of a chemical structure in any ratio, e.g., with enrichment (i.e., enantiomeric excess or diastereomeric excess) of one of the possible isomers and corresponding smaller ratios of other isomers. In addition, a crystal polymorphism may be present for the compounds represented by Formula (I-A), (II-A), (I), (II), or (III). It is noted that any crystal form, crystal form mixture, or anhydride or hydrate thereof is included in the scope of the present disclosure. Furthermore, so-called metabolite which is produced by degradation of the present compound in vivo is included in the scope of the present disclosure.

“Isomerism” means compounds that have identical molecular formulae but differ in the sequence of bonding of their atoms or in the arrangement of their atoms in space. Isomers that differ in the arrangement of their atoms in space are termed “stereoisomers”. Stereoisomers that are not mirror images of one another are termed “diastereoisomers”, and stereoisomers that are non-superimposable mirror images of each other are termed “enantiomers” or sometimes optical isomers. A mixture containing equal amounts of individual enantiomeric forms of opposite chirality is termed a “racemic mixture”.

A carbon atom bonded to four non-identical substituents is termed a “chiral center”.

“Chiral isomer” means a compound with at least one chiral center. Compounds with more than one chiral center may exist either as an individual diastereomer or as a mixture of diastereomers, termed “diastereomeric mixture”. When one chiral center is present, a stereoisomer may be characterized by the absolute configuration (R or S) of that chiral center. Absolute configuration refers to the arrangement in space of the substituents attached to the chiral center. The substituents attached to the chiral center under consideration are ranked in accordance with the Sequence Rule of Cahn, Ingold and Prelog. (Cahn et al., Angew. Chem. Inter. Edit. 1966, 5, 385; errata 511; Cahn et al., Angew. Chem. 1966, 78, 413; Cahn and Ingold, J. Chem. Soc. 1951 (London), 612; Cahn et al., Experientia 1956, 12, 81; Cahn, J. Chem. Educ. 1964, 41, 116).

Diastereoisomers, i.e., non-superimposable stereochemical isomers, can be separated by conventional means such as chromatography, distillation, crystallization, or sublimation. The optical isomers can be obtained by resolution of the racemic mixtures according to conventional processes, for example by formation of diastereoisomeric salts by treatment with an optically active acid or base. Examples of appropriate acids include, without limitation, tartaric, diacetyltartaric, dibenzoyltartaric, ditoluoyltartaric, and camphorsulfonic acid. The mixture of diastereomers can be separated by crystallization followed by liberation of the optically active bases from these salts. An alternative process for separation of optical isomers includes the use of a chiral chromatography column optimally chosen to maximize the separation of the enantiomers. Still another available method involves synthesis of covalent diastereoisomeric molecules by reacting compounds of Formula (I-A), (II-A), (I), (II), or (III) with an optically pure acid in an activated form or an optically pure isocyanate. The synthesized diastereoisomers can be separated by conventional means such as chromatography, distillation, crystallization or sublimation, and then hydrolyzed to obtain the enantiomerically pure compound. The optically active compounds of Formula (I-A), (II-A), (I), (II), or (III) can likewise be obtained by utilizing optically active starting materials and/or by utilizing a chiral catalyst. These isomers may be in the form of a free acid, a free base, an ester or a salt. Examples of chiral separation techniques are given in Chiral Separation Techniques, A Practical Approach, 2^{nd} ed. by G. Subramanian, Wiley-VCH, 2001.

“Geometric isomer” means the diastereomers that owe their existence to hindered rotation about double bonds. These configurations are differentiated in their names by the prefixes cis and trans, or Z and E, which indicate that the groups are on the same or opposite side of the double bond in the molecule according to the Cahn-Ingold-Prelog rules.

Furthermore, the structures and other compounds discussed in this disclosure include all atropic isomers thereof. “Atropic isomers” are a type of stereoisomer in which the atoms of two isomers are arranged differently in space. Atropic isomers owe their existence to a restricted rotation caused by hindrance of rotation of large groups about a central bond. Such atropic isomers typically exist as a mixture, however as a result of recent advances in chromatography techniques; it has been possible to separate mixtures of two atropic isomers in select cases.

“Tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solid form, usually one tautomer predominates. In solutions where tautomerization is possible, a chemical equilibrium of the tautomers will be reached. The exact ratio of the tautomers depends on several factors, including temperature, solvent, and pH. The concept of tautomers that are interconvertable by tautomerizations is called tautomerism.

Of the various types of tautomerism that are possible, two are commonly observed. In keto-enol tautomerism a simultaneous shift of electrons and a hydrogen atom occurs. Ring-chain tautomerism arises as a result of the aldehyde group (—CHO) in a sugar chain molecule reacting with one of the hydroxy groups (—OH) in the same molecule to give it a cyclic (ring-shaped) form as exhibited by glucose.

Common tautomeric pairs are: ketone-enol, amide-nitrile, lactam-lactim, amide-imidic acid tautomerism in heterocyclic rings (e.g., in nucleobases such as guanine, thymine, and cytosine), amine-enamine and enamine-enamine. It is to be understood that the compounds of the present disclosure may be depicted as different tautomers. It should also be understood that when compounds have tautomeric forms, all tautomeric forms are intended to be included in the scope of the present disclosure, and the naming of the compounds does not exclude any tautomer form.

Additionally, the compounds of the present disclosure, for example, the salts of the compounds, can exist in either hydrated or unhydrated (the anhydrous) form or as solvates with other solvent molecules. Nonlimiting examples of hydrates include monohydrates, dihydrates, etc. Nonlimiting examples of solvates include ethanol solvates, acetone solvates, etc.

“Solvate” means solvent addition forms that contain either stoichiometric or non-stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O.

As used herein, a “subject” or “subject in need thereof” is a subject having a disease or disorder associated with modulating of NR2F6. A “subject” includes a mammal. The mammal can be e.g., any mammal, e.g., a human, primate, bird, mouse, rat, fowl, dog, cat, cow, horse, goat, camel, sheep, or a pig. Preferably, the mammal is a human.

The present disclosure is intended to include all isotopes of atoms occurring in the present compounds. Isotopes include those atoms having the same atomic number but different mass numbers. By way of general example and without limitation, isotopes of hydrogen include tritium and deuterium, and isotopes of carbon include C-13 and C-14.

### Compounds

The present disclosure provides a compound represented by Formula (I-A) or (II-A):

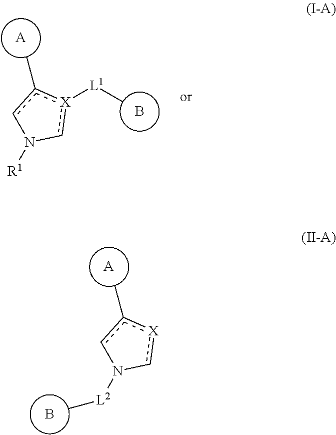

and pharmaceutically acceptable salts and tautomers thereof, wherein:

each [symbol] independently represents a single bond or a double bond;

X is N, NH, C, CH, or CH₂;

R¹ is H, C₁₋₆alkyl, cycloalkyl, heterocyclyl, —C(O)R^{1a}, —CH₂-aryl, —CH₂-heteroaryl, aryl, or heteroaryl; wherein R^{1a} is C₁₋₆alkyl; and wherein —CH₂-aryl, —CH₂-heteroaryl, aryl, and heteroaryl are optionally substituted with C₁₋₆alkyl or halo;

A is alkyl, cycloalkyl, heterocyclyl, a fused bicyclic aryl, a fused bicyclic heteroaryl, —CH₂-aryl, —CH₂-heteroaryl, aryl, or heteroaryl; wherein the aryl or heteroaryl is optionally substituted with aryl, heteroaryl, —Y^{A}-aryl, or —Y^{A}-heteroaryl; wherein Y^{A} is —O—, —C(O)—, —N(R^{A1})—, S(O)—, or —S(O)₂—; wherein R^{A1} is H or C₁₋₆alkyl;
wherein the fused bicyclic aryl, the fused bicyclic heteroaryl, —CH₂-aryl, —CH₂— heteroaryl, each aryl, and each heteroaryl are optionally substituted with one or more substituents selected from the group consisting of alkyl, halo, haloalkyl, —CN, —N(R^{A})₂, —OH, and —O-alkyl; wherein each R^{A} is independently H or C₁₋₆alkyl;

L¹ is —C(O)—NR^{L1}—, —O—C(S)—NR^{L1}—, —O—C(O)—NR^{L1}—, —NR^{L1}—C(O)—, —NR^{L1}—C(O)—O—, —NH—C(O)—NH—, —NR^{L1}—C(S)—NR^{L1}—, —NR^{L1}—S(O)₂—, —S(O)₂—NR^{L1}—, —CH₂—CH₂—, —CH₂—NR^{L1}—, —NR^{L1}—CH₂—, —CH₂—O—, —O—CH₂—, —O—, —NH—, —C(O)-azetidinyl, —CH₂—NR^{L1}—C(O)—, —C(O)—NR^{L1}—CH₂—, or —C(O)—; wherein each R^{L1} is independently H or C₁₋₆alkyl; and

L² is —C(O)—NR^{L2}—, —S(O)₂—NR^{L2}—, —CH₂—CH₂—, —C(S)—NR^{L2}—, —C(O)—, or —S(O)₂—; wherein each R^{L2} is independently H or C₁₋₆alkyl; and

B is a fused bicyclic aryl, a fused bicyclic heteroaryl, —CH₂-aryl, —CH₂-heteroaryl, aryl, heteroaryl, cycloalkyl, —CH₂-heterocyclyl, or heterocyclyl, wherein the aryl, heteroaryl, cycloalkyl, or heterocyclyl is optionally substituted with aryl, heteroaryl, —Y^{B}-aryl, —Y^{B}— heteroaryl, —Y^{B}-heterocyclyl, or cycloalkyl; wherein Y^{B} is —O—, —CH₂—, —C(O)—, —N(R^{B1})—, —S(O)—, or —S(O)₂—; wherein R^{B1} is H or C₁₋₆alkyl;
wherein the fused bicyclic aryl, the fused bicyclic heteroaryl, —CH₂-aryl, —CH₂-heteroaryl, each aryl, each heteroaryl, each cycloalkyl, —CH₂-heterocyclyl, and each heterocyclyl are optionally substituted with one or more substituents selected from the group consisting of alkyl, halo, haloalkyl, —CN, —N(R^{B2})₂, —OH, —O-alkyl, and oxo;

wherein each R^{B2} is independently H or C₁₋₆alkyl;

wherein when the compound is Formula (I-A); A is phenyl, and L¹ is —C(O)—NH—; then B is not

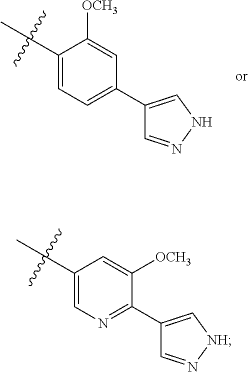 [SMILES: *c1ccc(-c2cn[nH]c2)cc1OC ; *c1cnc(-c2cn[nH]c2)c(OC)c1]

wherein when the compound is Formula (I-A); A is a substituted phenyl and B is a substituted phenyl, then L¹ is not —C(O)—NH—, —NH—C(O)—, —NCH₃—C(O)—, or —NH—C(O)—NH—;

wherein when the compound is Formula (I-A); L¹ is —C(O)—NR^{L1}—CH₂— and B is an optionally substituted phenyl, substituted pyridyl, or

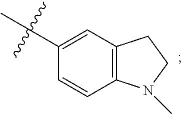 [SMILES: *c1ccc2c(c1)CCN2C]

then A is not substituted phenyl, substituted pyridyl, substituted thiophenyl, substituted thiazolyl, substituted pyrazolyl,

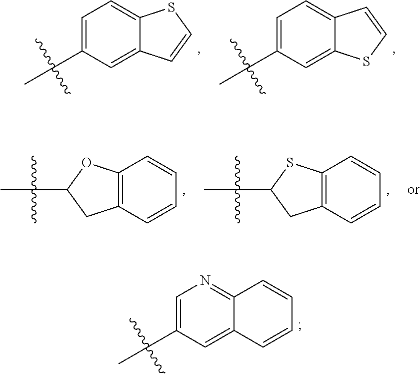 [SMILES: *c1ccc2sccc2c1 ; *c1ccc2ccsc2c1 ; *C1Cc2ccccc2O1 ; *C1Cc2ccccc2S1 ; *c1cnc2ccccc2c1]

wherein when the compound is Formula (I-A); B is optionally substituted —CH₂-aryl and A is optionally substituted aryl; then L¹ is not —C(O)—NH—;

wherein when the compound is Formula (II-A); A is optionally substituted phenyl and B is optionally substituted phenyl, then L¹ is not-C(O)—NCH₃—.

The present disclosure provides a compound represented by Formula (I) or (II):

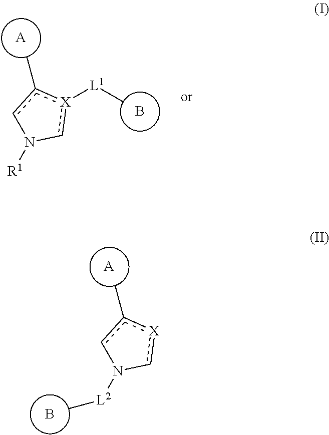

and pharmaceutically acceptable salts and tautomers thereof, wherein:

each [symbol] independently represents a single bond or a double bond;

X is N, NH, C, CH, or CH₂;

R¹ is H, C₁₋₆alkyl, cycloalkyl, heterocyclyl, —C(O)R^{1a}, —CH₂-aryl, —CH₂-heteroaryl, aryl, or heteroaryl; wherein R^{1a} is C₁₋₆alkyl; and wherein —CH₂-aryl, —CH₂-heteroaryl, aryl, and heteroaryl are optionally substituted with C₁₋₆alkyl or halo;

A is alkyl, cycloalkyl, heterocyclyl, a fused bicyclic aryl, a fused bicyclic heteroaryl, —CH₂-aryl, —CH₂-heteroaryl, aryl, or heteroaryl; wherein the aryl or heteroaryl is optionally substituted with aryl, heteroaryl, —Y^{A}-aryl, or —Y^{A}-heteroaryl; wherein Y^{A} is —O—, —C(O)—, —N(R^{A1})—, —S(O)—, or —S(O)₂—; wherein R^{A1} is H or C₁₋₆alkyl;
wherein the fused bicyclic aryl, the fused bicyclic heteroaryl, —CH₂-aryl, —CH₂— heteroaryl, each aryl, and each heteroaryl are optionally substituted with one or more substituents selected from the group consisting of alkyl, halo, —CN, —N(R^{A})₂, —OH, and —O-alkyl; wherein each R^{A} is independently H or C₁₋₆alkyl;

L¹ is —C(O)—NR^{L1}—, —O—C(S)—NR^{L1}—, —O—C(O)—NR^{L1}—, —NR^{L1}—C(O)—, —NR^{L1}—C(O)—O—, —NH—C(O)—NH—, —NR^{L1}—C(S)—NR^{L1}—, —NR^{L1}—S(O)₂—, —S(O)₂—NR^{L1}—, —CH₂—CH₂—, —CH₂—NR^{L1}—, —NR^{L1}—CH₂—, —CH₂—O—, —O—CH₂—, —O—, —NH—, —C(O)-azetidinyl, —CH₂—NR^{L1}—C(O)—, or —C(O)—NR^{L1}—CH₂—; wherein each R^{L1} is independently H or C₁₋₆alkyl; and

L² is —C(O)—NR^{L2}—, —S(O)₂—NR^{L2}—, —CH₂—CH₂—, —C(S)—NR^{L2}—, —C(O)—, or —S(O)₂—; wherein each R^{L2} is independently H or C₁₋₆alkyl; and

B is a fused bicyclic aryl, a fused bicyclic heteroaryl, —CH₂-aryl, —CH₂-heteroaryl, aryl, heteroaryl, cycloalkyl, or —CH₂-heterocyclyl, wherein the aryl or heteroaryl is optionally substituted with aryl, heteroaryl, —Y^{B}-aryl, or —Y^{B}-heteroaryl; wherein Y^{B} is —O—, —C(O)—, —N(R^{B1})—, —S(O)—, or —S(O)₂—; wherein R^{B1} is H or C₁₋₆alkyl;
wherein the fused bicyclic aryl, the fused bicyclic heteroaryl, —CH₂-aryl, —CH₂-heteroaryl, each aryl, each heteroaryl, cycloalkyl, and —CH₂-heterocyclyl are optionally substituted with one or more substituents selected from the group consisting of alkyl, halo, —CN, —N(R^{B2})₂, —OH, and —O-alkyl; wherein each R^{B2} is independently H or C₁₋₆alkyl;

wherein when the compound is Formula (I); A is optionally substituted phenyl or thiophenyl, and L¹ is —C(O)—NH—; then B is not

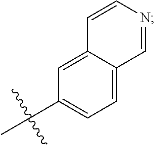 [SMILES: *c1ccc2cnccc2c1]

wherein when the compound is Formula (I); A is a substituted phenyl and B is a substituted phenyl, then L¹ is not —C(O)—NH—, —NH—C(O)—, —NCH₃—C(O)—, or —NH—C(O)—NH—;

wherein when the compound is Formula (I); B is optionally substituted —CH₂-aryl and A is optionally substituted aryl; then L¹ is not —C(O)—NH—;

wherein when the compound is Formula (II); A is optionally substituted phenyl and B is optionally substituted phenyl, then L¹ is not —C(O)—NCH₃—.

In certain embodiments, when the compound is Formula (I-A) or (I), A is a substituted phenyl, and L¹ is —CH₂—O—; then B is not

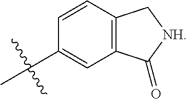 [SMILES: *c1ccc2c(c1)C(=O)NC2]

In certain embodiments, when the compound is Formula (I); A is optionally substituted phenyl or thiophenyl, and L¹ is —C(O)—NH—; then B is not

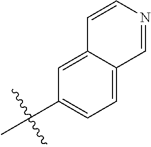 [SMILES: *c1ccc2cnccc2c1]

or

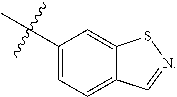 [SMILES: *c1ccc2cnsc2c1]

In certain embodiments, when the compound is Formula (I); A is phenyl, and L¹ is —C(O)—NH—; then B is not

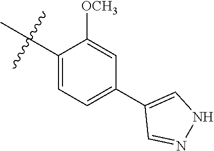 [SMILES: *c1ccc(-c2cn[nH]c2)cc1OC]

or

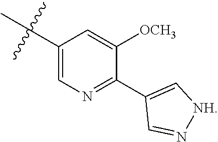 [SMILES: *c1cnc(-c2cn[nH]c2)c(OC)c1]

in certain embodiments, when the compound is Formula (I); L¹ is —C(O)—NR^{L1}—CH₂— and B is an optionally substituted phenyl, substituted pyridyl, or

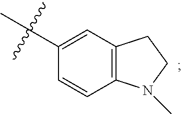 [SMILES: *c1ccc2c(c1)CCN2C]

then A is not substituted phenyl, substituted pyridyl, substituted thiophenyl, substituted thiazolyl, substituted pyrazolyl,

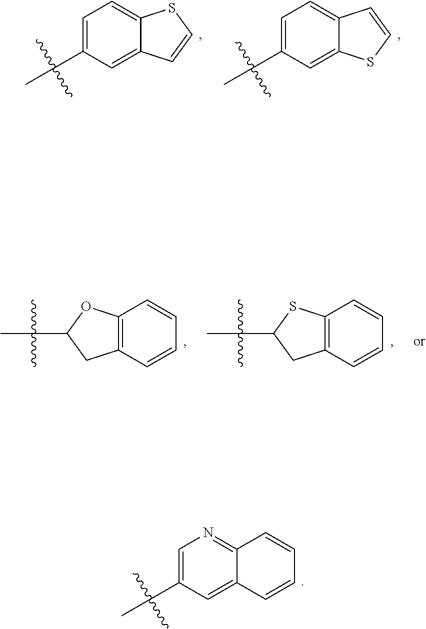 [SMILES: *c1ccc2sccc2c1 ; *c1ccc2ccsc2c1 ; *C1Cc2ccccc2O1 ; *C1Cc2ccccc2S1 ; *c1cnc2ccccc2c1]

The present disclosure provides a compound represented by Formula (III):

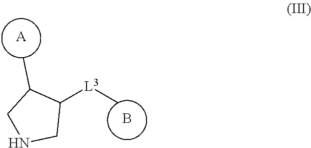

and pharmaceutically acceptable salts and tautomers thereof, wherein:

A is aryl or 5- to 6-membered heteroaryl, wherein the aryl and heteroaryl are optionally substituted with one or more substituents selected from the group consisting of alkyl, halo, —OH, and —O-alkyl;

L³ is —C(O)—NR^{L3}—, —O—C(S)—NR^{L3}—, —O—C(O)—NR^{L3}—, —NR^{L3}—C(O)—, —NR^{L3}—C(S)—NR^{L3}—, —NR^{L3}—S(O)₂—, —S(O)₂—NR^{L3}—, —CH₂—CH₂—, —CH₂—NR^{L3}—, —NR^{L3}—CH₂—, —CH₂—O—, —O—CH₂—, or —O—; wherein each R^{L3} is independently hydrogen or C₁₋₆alkyl; and

B is a fused bicyclic aryl, a fused bicyclic heteroaryl, —CH₂-aryl, —CH₂-heteroaryl, aryl, or heteroaryl, wherein the aryl or heteroaryl is optionally substituted with aryl or heteroaryl;
wherein the fused bicyclic aryl, the fused bicyclic heteroaryl, —CH₂-aryl, —CH₂— heteroaryl, each aryl, and each heteroaryl are optionally substituted with one or more substituents selected from the group consisting of alkyl, halo, —OH, and —O-alkyl;

wherein when A is optionally substituted phenyl or thiophenyl, and L³ is —C(O)—NH—; then B is not

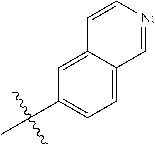 [SMILES: *c1ccc2cnccc2c1]

wherein when A is a substituted phenyl and B is a substituted phenyl, then L³ is not —C(O)—NH—, —NH—C(O)—, —NCH₃—C(O)—, or —NH—C(O)—NH—;

wherein when B is optionally substituted —CH₂-aryl and A is optionally substituted aryl; then L³ is not —C(O)—NH—.

In certain embodiments, when the compound is Formula (III), A is a substituted phenyl, and L³ is —CH₂—O—; then B is not

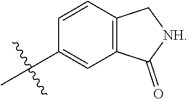 [SMILES: *c1ccc2c(c1)C(=O)NC2]

The present disclosure provides a compound represented by Formula (IV):

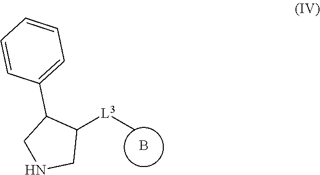

and pharmaceutically acceptable salts and tautomers thereof, wherein:

L³ is —C(O)—NR^{L3}—, —O—C(S)—NR^{L3}—, —O—C(O)—NR^{L3}—, —NR^{L3}—C(O)—, —NR^{L3}—C(S)—NR^{L3}—, —NR^{L3}—S(O)₂—, —S(O)₂—NR^{L3}—, —CH₂—CH₂—, —CH₂—NR^{L3}—, —NR^{L3}—CH₂—, —CH₂—O—, —O—CH₂—, or —O—; wherein each R^{L3} is independently hydrogen or C₁₋₆alkyl; and

B is a fused bicyclic aryl, a fused bicyclic heteroaryl, —CH₂-aryl, —CH₂-heteroaryl, aryl, or heteroaryl, wherein the aryl or heteroaryl is optionally substituted with aryl or heteroaryl;
wherein the fused bicyclic aryl, the fused bicyclic heteroaryl, —CH₂-aryl, —CH₂— heteroaryl, each aryl, and each heteroaryl are optionally substituted with one or more substituents selected from the group consisting of alkyl, halo, —OH, and —O-alkyl;

wherein when L³ is —C(O)—NH—; then B is not

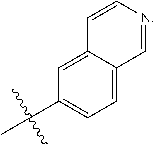 [SMILES: *c1ccc2cnccc2c1]

The present disclosure provides a compound represented by Formula (V):

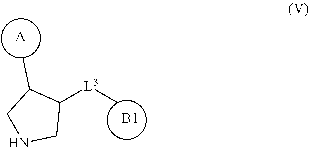

and pharmaceutically acceptable salts and tautomers thereof, wherein:

A is aryl or 5- to 6-membered heteroaryl, wherein the aryl and heteroaryl are optionally substituted with one or more substituents selected from the group consisting of alkyl, halo, —OH, and —O-alkyl;

L³ is —C(O)—NR^{L3}—, —O—C(S)—NR^{L3}—, —O—C(O)—NR^{L3}—, —NR^{L3}—C(O)—, —NR^{L3}—C(S)—NR^{L3}—, —NR^{L3}—S(O)₂—, —S(O)₂—NR^{L3}—, —CH₂—CH₂—, —CH₂—NR^{L3}—, —NR^{L3}—CH₂—, —CH₂—O—, —O—CH₂—, or —O—; wherein each R^{L3} is independently hydrogen or C₁₋₆alkyl; and

B1 is a fused bicyclic aryl or a fused bicyclic heteroaryl; wherein the fused bicyclic aryl and the fused bicyclic heteroaryl are optionally substituted with one or more substituents selected from the group consisting of alkyl, halo, —OH, and —O-alkyl;

wherein when A is optionally substituted phenyl or thiophenyl, and L³ is —C(O)—NH—; then B is not

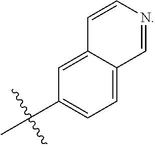 [SMILES: *c1ccc2cnccc2c1]

In certain embodiments, when the compound is Formula (V), A is a substituted phenyl, and L³ is —CH₂—O—; then B is not

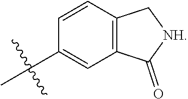 [SMILES: *c1ccc2c(c1)C(=O)NC2]

In certain embodiments of formula (V), B1 is a fused bicyclic aryl. In certain embodiments, B1 is a fused bicyclic heteroaryl. In certain embodiments, B1 is selected from the group consisting of

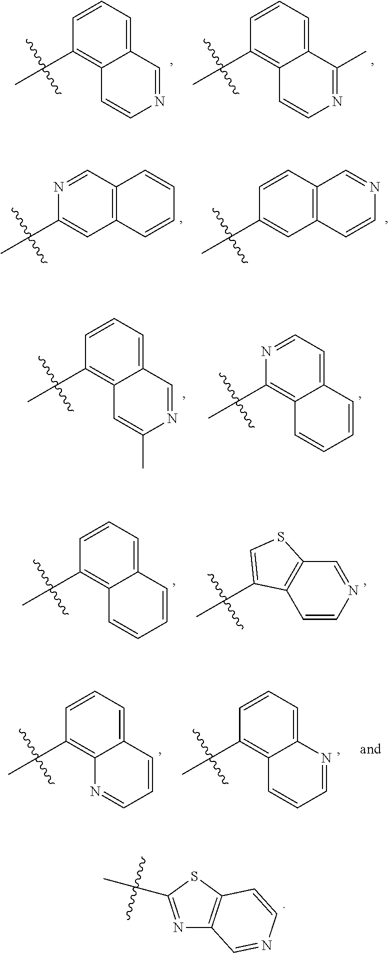

The present disclosure provides a compound represented by Formula (VI):

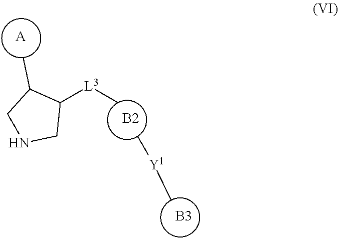

and pharmaceutically acceptable salts and tautomers thereof, wherein:

A is aryl or 5- to 6-membered heteroaryl, wherein the aryl and heteroaryl are optionally substituted with one or more substituents selected from the group consisting of alkyl, halo, —OH, and —O-alkyl;

L³ is —C(O)—NR^{L3}—, —O—C(S)—NR^{L3}—, —O—C(O)—NR^{L3}—, —NR^{L3}—C(O)—, —NR^{L3}—C(S)—NR^{L3}—, —NR^{L3}—S(O)₂—, —S(O)₂—NR^{L3}—, —CH₂—CH₂—, —CH₂—NR^{L3}—, —NR^{L3}—CH₂—, —CH₂—O—, —O—CH₂—, or —O—; wherein each R^{L3} is independently hydrogen or C₁₋₆alkyl; and

B2 is monocyclic aryl or monocyclic heteroaryl; wherein the aryl and heteroaryl are optionally substituted with one or more substituents selected from the group consisting of alkyl, halo, —OH, and —O-alkyl;

Y¹ is absent, —O—, —C(O)—, —N(R^{Y})—, —S(O)—, or —S(O)₂—; wherein R^{Y} is H or C₁₋₆alkyl; and

B3 is monocyclic aryl or monocyclic heteroaryl; wherein the aryl and heteroaryl are optionally substituted with one or more substituents selected from the group consisting of alkyl, halo, —OH, and —O-alkyl.

In certain embodiments of Formula (VI), B2 is monocyclic aryl. In certain embodiments, B2 is monocyclic heteroaryl. In certain embodiments, B3 is monocyclic aryl. In certain embodiments, B3 is monocyclic heteroaryl. In certain embodiments,

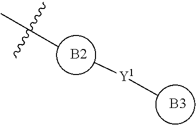

is selected from the group consisting of

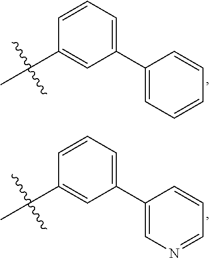 [SMILES: *c1cccc(-c2ccccc2)c1 ; *c1cccc(-c2cccnc2)c1]

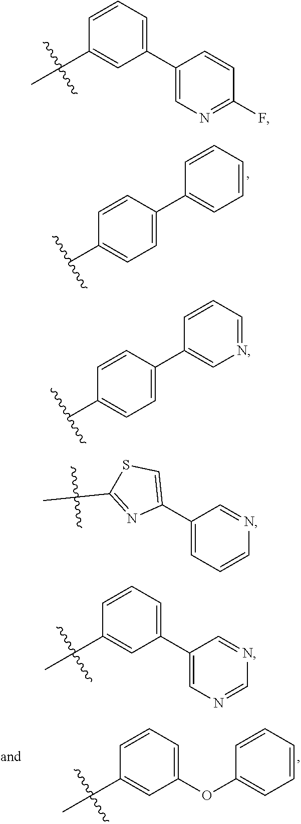

The present disclosure provides a compound represented by Formula (VII):

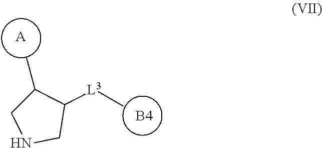

and pharmaceutically acceptable salts and tautomers thereof, wherein:

A is aryl or 5- to 6-membered heteroaryl, wherein the aryl and heteroaryl are optionally substituted with one or more substituents selected from the group consisting of alkyl, halo, —OH, and —O-alkyl;

L³ is —C(O)—NR^{L3}—, —O—C(S)—NR^{L3}—, —O—C(O)—NR^{L3}—, —NR^{L3}—C(O)—, —NR^{L3}—C(S)—NR^{L3}—, —NR^{L3}—S(O)₂—, —S(O)₂—NR^{L3}—, —CH₂—CH₂—, —CH₂—NR^{L3}—, —NR^{L3}—CH₂—, —CH₂—O—, —O—CH₂—, or —O—; wherein each R^{L3} is independently hydrogen or C₁₋₆alkyl; and

B4 is —CH₂-aryl or —CH₂-heteroaryl; wherein —CH₂-aryl and —CH₂-heteroaryl are optionally substituted with one or more substituents selected from the group consisting of alkyl, halo, —OH, and —O-alkyl;

wherein when B4 is optionally substituted —CH₂-aryl and A is optionally substituted aryl; then L³ is not —C(O)—NH—.

In certain embodiments of Formula (VII), B4 is —CH₂-aryl. In certain embodiments, B4 is —CH₂-heteroaryl. In certain embodiments, B4 is selected from the group consisting of

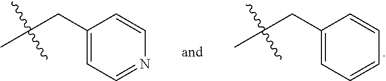 [SMILES: *Cc1ccncc1 ; *Cc1ccccc1]

As described above, Formula (I-A) or (I) is

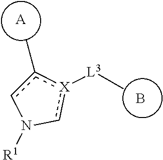

### and Formula (II-A) or (II) is

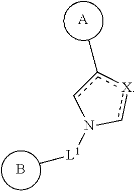

In certain embodiments, the compound is Formula (I-A) or (I). In certain embodiments, the compound is Formula (II-A) or (II).

In certain embodiments, Formula (I-A) or (I) has the following stereochemistry:

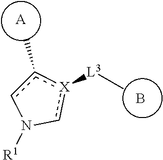

In certain embodiments, Formula (I-A) or (I) has the following stereochemistry:

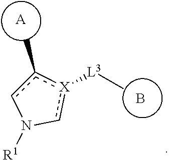

In certain embodiments, Formula (I-A) or (I) has the following stereochemistry:

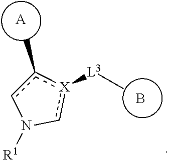

In certain embodiments, Formula (I-A) or (I) has the following stereochemistry:

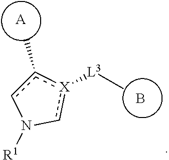

In certain embodiments

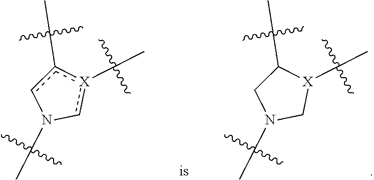

In certain embodiments

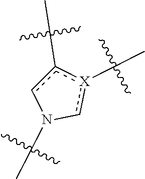

is

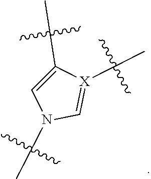

In certain embodiments,

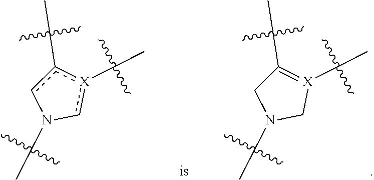

In certain embodiments, X is N or NH. In certain embodiments, X is C, CH, or CH₂.

As described above, R¹ is H, C₁₋₆alkyl, cycloalkyl, heterocyclyl, —C(O)R^{1a}, —CH₂-aryl, —CH₂-heteroaryl, aryl, or heteroaryl; wherein R^{1a} is C₁₋₆alkyl; and wherein —CH₂-aryl, —CH₂-heteroaryl, aryl, and heteroaryl are optionally substituted with C₁₋₆alkyl or halo.

In certain embodiments, R¹ is H. In certain embodiments, R¹ is C₁₋₆alkyl. In certain embodiments, R¹ is cycloalkyl. In certain embodiments, R¹ is heterocyclyl. In certain embodiments, R¹ is —C(O)R^{1a}. In certain embodiments, R¹ is —C(O)R^{1a}, wherein R^{1a} is C₁₋₆alkyl. In certain embodiments, R¹ is —CH₂-aryl. In certain embodiments, R¹ is —CH₂— heteroaryl. In certain embodiments, R¹ is aryl. In certain embodiments, R¹ is heteroaryl.

As described above, A is alkyl, cycloalkyl, heterocyclyl, a fused bicyclic aryl, a fused bicyclic heteroaryl, —CH₂-aryl, —CH₂-heteroaryl, aryl, or heteroaryl; wherein the aryl or heteroaryl is optionally substituted with aryl, heteroaryl, —Y^{A}-aryl, or —Y^{A}-heteroaryl; wherein Y^{A} is —O—, —C(O)—, —N(R^{A1})—, —S(O)—, or —S(O)₂—; wherein R^{A1} is H or C₁₋₆alkyl; wherein the fused bicyclic aryl, the fused bicyclic heteroaryl, —CH₂-aryl, —CH₂-heteroaryl, each aryl, and each heteroaryl are optionally substituted with one or more substituents selected from the group consisting of alkyl, halo, —CN, —N(R^{A})₂, —OH, and —O-alkyl; wherein each R^{A} is independently H or C₁₋₆ alkyl.

In certain embodiments, A is alkyl. In certain embodiments, A is cycloalkyl. In certain embodiments, A is heterocyclyl. In certain embodiments, A is a fused bicyclic aryl. In certain embodiments, A is a fused bicyclic heteroaryl. In certain embodiments, A is —CH₂-aryl. In certain embodiments, A is —CH₂-heteroaryl. In certain embodiments, A is aryl. In certain embodiments, the aryl is substituted with one or more substituents selected from the group consisting of alkyl, halo, —OH, and —O-alkyl. In certain embodiments, A is 5- to 6-membered heteroaryl. In certain embodiments, the heteroaryl is substituted with one or more substituents selected from the group consisting of alkyl, halo, —OH, and —O-alkyl.

In certain embodiments, A is aryl. In certain embodiments, the aryl is unsubstituted. In certain embodiments, the aryl of A ring is optionally substituted with aryl, heteroaryl, —Y^{A}-aryl, or —Y^{A}-heteroaryl, wherein Y^{A} is —O—, —C(O)—, —N(R^{A1})—, —S(O)—, or —S(O)₂—. In certain embodiments, the aryl is substituted with aryl. In certain embodiments, the aryl is substituted with heteroaryl. In certain embodiments, the aryl is substituted with —Y^{A}-aryl. In certain embodiments, the aryl is substituted with —Y^{A}-heteroaryl. In certain embodiments, Y^{A} is —O—. In certain embodiments, Y^{A} is —C(O)—. In certain embodiments, Y^{A} is —N(R^{A1})—. In certain embodiments, Y^{A} is —S(O)—. In certain embodiments, Y^{A} is —S(O)₂—.

In certain embodiments, A is heteroaryl. In certain embodiments, the heteroaryl is unsubstituted. In certain embodiments, the heteroaryl of A ring is optionally substituted with aryl, heteroaryl, —Y^{A}-aryl, or —Y^{A}-heteroaryl, wherein Y^{A} is —O—, —C(O)—, —N(R^{A1})—, —S(O)—, or —S(O)₂—. In certain embodiments, the heteroaryl is substituted with aryl. In certain embodiments, the heteroaryl is substituted with heteroaryl. In certain embodiments, the heteroaryl is substituted with —Y^{A}-aryl. In certain embodiments, the heteroaryl is substituted with —Y^{A}-heteroaryl. In certain embodiments, Y^{A} is —O—. In certain embodiments, Y^{A} is —C(O)—. In certain embodiments, Y^{A} is —N(R^{A1})—. In certain embodiments, Y^{A} is —S(O)—. In certain embodiments, Y^{A} is —S(O)₂—.

In certain embodiments, A is a monocyclic aryl or a monocyclic heteroaryl; wherein the monocyclic aryl or the monocyclic heteroaryl is substituted with aryl or heteroaryl. For example, in certain embodiments, A is a monocyclic aryl substituted with an aryl. For example, in certain embodiments, A is a monocyclic aryl substituted with a heteroaryl. For example, in certain embodiments, A is a monocyclic heteroaryl substituted with an aryl. For example, in certain embodiments, A is a monocyclic heteroaryl substituted with a heteroaryl. In certain embodiments, the monocyclic aryl, monocyclic heteroaryl, aryl, or heteroaryl are optionally substituted with one or more substituents selected from the group consisting of alkyl, halo, —OH, and —O-alkyl.

In certain embodiments, A is a fused bicyclic aryl. Fused bicyclic aryl refers to a polycyclic group with two fused rings having at least one hydrocarbon aromatic ring, wherein all of the ring atoms of the at least one hydrocarbon aromatic ring are carbon. In certain embodiments, fused bicyclic aryl comprises two aromatic rings.

In certain embodiments, A is a fused bicyclic heteroaryl. Fused bicyclic heteroaryl refers to a polycyclic group with two fused rings comprising at least one aromatic ring, wherein the aromatic ring comprises at least one ring heteroatom independently selected from the group consisting of N, O, and S. In certain embodiments, fused bicyclic heteroaryl comprises two aromatic rings.

As described above for Formula (III), A is aryl or 5- to 6-membered heteroaryl, wherein the aryl and heteroaryl are optionally substituted with one or more substituents selected from the group consisting of alkyl, halo, —OH, and —O-alkyl.

In certain embodiments, A is aryl. In certain embodiments, A is phenyl. In certain embodiments, A is a 5- to 6-membered heteroaryl. In certain embodiments, A is a 5-membered heteroaryl. In certain embodiments, A is a 5-membered heteroaryl containing S. In certain embodiments, A is a 6-membered heteroaryl.

As described above for A, the fused bicyclic aryl, the fused bicyclic heteroaryl, —CH₂-aryl, —CH₂-heteroaryl, each aryl, and each heteroaryl are optionally substituted with one or more substituents selected from the group consisting of alkyl, halo, —CN, —N(R^{A})₂, —OH, and —O-alkyl; wherein each R^{A} is independently H or C₁₋₆ alkyl.

As described above for A, the fused bicyclic aryl, the fused bicyclic heteroaryl, —CH₂-aryl, —CH₂-heteroaryl, each aryl, and each heteroaryl are optionally substituted with one or more substituents selected from the group consisting of alkyl, halo, haloalkyl, —CN, —N(R^{A})₂, —OH, and —O-alkyl; wherein each R^{A} is independently H or C₁₋₆alkyl.

As described above for formula (I), L¹ is —C(O)—NR^{L1}—, —O—C(S)—NR^{L1}—, —O—C(O)—NR^{L1}—, —NR^{L1}—C(O)—, —NR^{L1}—C(O)—O—, —NH—C(O)—NH—, —NR^{L1}—C(S)—NR^{L1}—, —NR^{L1}—S(O)₂—, —S(O)₂—NR^{L1}—, —CH₂—CH₂—, —CH₂—NR^{L1}—, —NR^{L1}—CH₂—, —CH₂—O—, —O—CH₂—, —O—, —NH—, —C(O)-azetidinyl, —CH₂—NR^{L1}—C(O)—, or —C(O)—NR^{L1}—CH₂—; wherein each R^{L1} is independently H or C₁₋₆alkyl. As described above for formula (I-A), L¹ is —C(O)—NR^{L1}—, —O—C(S)—NR^{L1}—, —O—C(O)—NR^{L1}—, —NR^{L1}—C(O)—, —NR^{L1}—C(O)—O—, —NH—C(O)—NH—, —NR^{L1}—C(S)—NR^{L1}—, —NR^{L1}—S(O)₂—, —S(O)₂—NR^{L1}—, —CH₂—CH₂—, —CH₂—NR^{L1}—, —NR^{L1}—CH₂—, —CH₂—O—, —O—CH₂—, —O—, —NH—, —C(O)-azetidinyl, —CH₂—NR^{L1}—C(O)—, —C(O)—NR^{L1}—CH₂—, or —C(O)—; wherein each R^{L1} is independently H or C₁₋₆alkyl.

In certain embodiments, L¹ is —C(O)—NR^{L1}—. In certain embodiments, L¹ is —O—C(S)—NR^{L1}—. In certain embodiments, L¹ is —O—C(O)—NR^{L1}—. In certain embodiments, L¹ is —NR^{L1}—C(O)—. In certain embodiments, L¹ is —NR^{L1}—C(O)—O—. In certain embodiments, L¹ is —NR^{L1}—C(O)—NR^{L1}—. In certain embodiments, L¹ is —NR^{L1}—C(S)—NR^{L1}—. In certain embodiments, L¹ is —NR^{L1}—S(O)₂—. In certain embodiments, L¹ is —S(O)₂—NR^{L1}—. In certain embodiments, L¹ is —CH₂—CH₂—. In certain embodiments, L¹ is —CH₂—NR^{L1}—. In certain embodiments, L¹ is —NR^{L1}—CH₂—. In certain embodiments, L¹ is —CH₂—O—. In certain embodiments, L¹ is —O—CH₂—. In certain embodiments, L¹ is —O—. In certain embodiments, L¹ is —NH—. In certain embodiments, L¹ is —C(O)-azetidinyl. In certain embodiments, L¹ is —CH₂—NR^{L1}—C(O)—. In certain embodiments, L¹ is —C(O)—NR^{L1}—CH₂—. In certain embodiments, L¹ is —C(O)—.

In certain embodiments, L¹ is —C(O)—NH—. In certain embodiments, L¹ is —O—C(S)—NH— In certain embodiments, L¹ is —O—C(O)—NH—. In certain embodiments, L¹ is —NH—C(O)—. In certain embodiments, L¹ is —NH—C(O)—O—. In certain embodiments, L¹ is —NH—C(O)—NH—. In certain embodiments, L¹ is —NH—C(S)—NH—. In certain embodiments, L¹ is —NH—S(O)₂—. In certain embodiments, L¹ is —S(O)₂—NH—. In certain embodiments, L¹ is —CH₂—CH₂—. In certain embodiments, L¹ is —CH₂—NH—. In certain embodiments, L¹ is —NH—CH₂—. In certain embodiments, L¹ is —CH₂—O—. In certain embodiments, L¹ is —O—CH₂—. In certain embodiments, L¹ is —O—. In certain embodiments, L¹ is —NH—. In certain embodiments, L¹ is —C(O)-azetidinyl. In certain embodiments, L¹ is —CH₂—NH—C(O)—. In certain embodiments, L¹ is —C(O)—NH—CH₂—.

As described above for formula (II), L² is —C(O)—NR^{L2}—, —S(O)₂—NR^{L2}—, —CH₂—CH₂—, —C(S)—NR^{L2}—, —C(O)—, or —S(O)₂—; wherein each R^{L2} is independently H or C₁₋₆alkyl.

In certain embodiments, L² is —C(O)—NR^{L2}—. In certain embodiments, L² is —S(O)₂—NR^{L2}—. In certain embodiments, L² is —CH₂—CH₂. In certain embodiments, L² is —C(S)—NR^{L2}—. In certain embodiments, L² is —C(O)—. In certain embodiments, L² is —S(O)₂—.

In certain embodiments, L² is —C(O)—NH—. In certain embodiments, L² is —S(O)₂—NH—. In certain embodiments, L² is —CH₂—CH₂. In certain embodiments, L² is —C(S)—NH—. In certain embodiments, L² is —C(O)—. In certain embodiments, L² is —S(O)₂—.

As described above for formula (III)-(VII), L³ is —C(O)—NR^{L3}—, —O—C(S)—NR^{L3}—, —O—C(O)—NR^{L3}—, —NR^{L3}—C(O)—, —NR^{L3}—C(S)—NR^{L3}—, —NR^{L3}—S(O)₂—, —S(O)₂—NR^{L3}—, —CH₂—CH₂—, —CH₂—NR^{L3}—, —NR^{L3}—CH₂—, —CH₂—O—, —O—CH₂—, or —O—; wherein each R^{L3} is independently hydrogen or C₁₋₆alkyl.

In certain embodiments, L³ is —C(O)—NR^{L3}—. In certain embodiments, L³ is —O—C(S)—NR^{L3}—. In certain embodiments, L³ is —O—C(O)—NR^{L3}—. In certain embodiments, L³ is —NR^{L3}—C(O)—. In certain embodiments, L³ is —NR^{L3}—C(S)—NR^{L3}—. In certain embodiments, L³ is —NR^{L3}—S(O)₂—. In certain embodiments, L³ is —S(O)₂—NR^{L3}—. In certain embodiments, L³ is —CH₂—CH₂—. In certain embodiments, L³ is —CH₂—NR^{L3}—. In certain embodiments, L³ is —NR^{L3}—CH₂—. In certain embodiments, L³ is —CH₂—O—. In certain embodiments, L³ is —O—CH₂—. In certain embodiments, L³ is —O—.

In certain embodiments, L³ is —C(O)—NH—. In certain embodiments, L³ is —O—C(S)—NH—. In certain embodiments, L³ is —O—C(O)—NH—. In certain embodiments, L³ is —NH—C(O)—. In certain embodiments, L³ is —NH—C(S)—NH—. In certain embodiments, L³ is —NH—S(O)₂—. In certain embodiments, L³ is —S(O)₂—NH—. In certain embodiments, L³ is —CH₂—CH₂—. In certain embodiments, L³ is —CH₂—NH—. In certain embodiments, L³ is —NH—CH₂—. In certain embodiments, L³ is —CH₂—O—. In certain embodiments, L³ is —O—CH₂—. In certain embodiments, L³ is —O—.

As described above, B is a fused bicyclic aryl, a fused bicyclic heteroaryl, —CH₂-aryl, —CH₂-heteroaryl, aryl, heteroaryl cycloalkyl, or —CH₂-heterocyclyl, wherein the aryl or heteroaryl is optionally substituted with aryl, heteroaryl, —Y^{B}-aryl, or —Y^{B}-heteroaryl; wherein Y^{B} is —O—, —C(O)—, —N(R^{B1})—, —S(O)—, or —S(O)₂—; wherein R^{B1} is H or C₁₋₆alkyl; wherein the fused bicyclic aryl, the fused bicyclic heteroaryl, —CH₂-aryl, —CH₂-heteroaryl, each aryl, each heteroaryl, cycloalkyl, and —CH₂-heterocyclyl are optionally substituted with one or more substituents selected from the group consisting of alkyl, halo, —CN, —N(R^{B2})₂, —OH, and —O— alkyl; wherein each R^{B2} is independently H or C₁₋₆alkyl. As described above for Formula (I-A), B is a fused bicyclic aryl, a fused bicyclic heteroaryl, —CH₂-aryl, —CH₂-heteroaryl, aryl, heteroaryl, cycloalkyl, —CH₂-heterocyclyl, or heterocyclyl, wherein the aryl, heteroaryl, cycloalkyl, or heterocyclyl is optionally substituted with aryl, heteroaryl, —Y^{B}-aryl, —Y^{B}— heteroaryl, —Y^{B}-heterocyclyl, or cycloalkyl; wherein Y^{B} is —O—, —CH₂—, —C(O)—, —N(R^{B1})—, —S(O)—, or —S(O)₂—; wherein R^{B1} is H or C₁₋₆alkyl; wherein the fused bicyclic aryl, the fused bicyclic heteroaryl, —CH₂-aryl, —CH₂-heteroaryl, each aryl, each heteroaryl, each cycloalkyl, —CH₂-heterocyclyl, and each heterocyclyl are optionally substituted with one or more substituents selected from the group consisting of alkyl, halo, haloalkyl, —CN, —N(R^{B2})₂, —OH, —O-alkyl, and oxo; wherein each R^{B2} is independently H or C₁₋₆alkyl

As described above for Formula (III), B is a fused bicyclic aryl, a fused bicyclic heteroaryl, —CH₂-aryl, —CH₂-heteroaryl, aryl, or heteroaryl, wherein the aryl or heteroaryl is optionally substituted with aryl or heteroaryl; wherein the fused bicyclic aryl, the fused bicyclic heteroaryl, —CH₂-aryl, —CH₂-heteroaryl, each aryl, and each heteroaryl are optionally substituted with one or more substituents selected from the group consisting of alkyl, halo, —OH, and —O-alkyl.

In certain embodiments, B is a fused bicyclic aryl. In certain embodiments, B is a fused bicyclic heteroaryl. In certain embodiments, B is —CH₂-aryl. In certain embodiments, B is —CH₂-heteroaryl. In certain embodiments, B is aryl. In certain embodiments, B is heteroaryl. In certain embodiments, B is cycloalkyl. In certain embodiments, B is —CH₂-heterocyclyl.

In certain embodiments, B is a fused bicyclic aryl. Fused bicyclic aryl refers to a polycyclic group with two fused rings having at least one hydrocarbon aromatic ring, wherein all of the ring atoms of the at least one hydrocarbon aromatic ring are carbon. In certain embodiments, fused bicyclic aryl comprises two aromatic rings. In certain embodiments, fused bicyclic aryl comprises an aromatic ring and a non-aromatic ring.

In certain embodiments, B is a fused bicyclic heteroaryl. Fused bicyclic heteroaryl refers to a polycyclic group with two fused rings comprising at least one aromatic ring, wherein the aromatic ring comprises at least one ring heteroatom independently selected from the group consisting of N, O, and S. In certain embodiments, fused bicyclic heteroaryl comprises two aromatic rings.

In certain embodiments, B is aryl. In certain embodiments, the aryl of B ring is optionally substituted with aryl, heteroaryl, —Y^{B}-aryl, or —Y^{B}-heteroaryl, wherein Y^{B} is —O—, —C(O)—, —N(R^{A1})—, —S(O)—, or —S(O)₂—. In certain embodiments, the aryl is unsubstituted. In certain embodiments, the aryl is substituted with aryl. In certain embodiments, the aryl is substituted with heteroaryl. In certain embodiments, the aryl is substituted with —Y^{B}-aryl. In certain embodiments, the aryl is substituted with —Y^{B}-heteroaryl. In certain embodiments, the aryl is substituted with —Y^{B}-heterocyclyl. In certain embodiments, the aryl is substituted with cycloalkyl. In certain embodiments, Y^{B} is —O—. In certain embodiments, Y^{B} is —C(O)—. In certain embodiments, Y^{B} is —N(R^{B1})—. In certain embodiments, Y^{B} is —S(O)—. In certain embodiments, Y^{B} is —S(O)₂—. In certain embodiments, Y^{B} is —CH₂—.

In certain embodiments, B is heteroaryl. In certain embodiments, the heteroaryl of B ring is optionally substituted with aryl, heteroaryl, —Y^{B}-aryl, or —Y^{B}-heteroaryl, Y^{B} is —O—, —C(O)—, —N(R^{B1})—, —S(O)—, or —S(O)₂—. In certain embodiments, the heteroaryl is unsubstituted. In certain embodiments, the heteroaryl is substituted with aryl. In certain embodiments, the heteroaryl is substituted with heteroaryl. In certain embodiments, the heteroaryl is substituted with —Y^{B}-aryl. In certain embodiments, the heteroaryl is substituted with —Y^{B}-heteroaryl. In certain embodiments, the heteroaryl is substituted with —Y^{B}— heterocyclyl. In certain embodiments, the heteroaryl is substituted with cycloalkyl. In certain embodiments, Y^{B} is —O—. In certain embodiments, Y^{B} is —C(O)—. In certain embodiments, Y^{B} is —N(R^{B1})—. In certain embodiments, Y^{B} is —S(O)—. In certain embodiments, Y^{B} is —S(O)₂—. In certain embodiments, Y^{B} is —CH₂—.

In certain embodiments, B is a monocyclic aryl or a monocyclic heteroaryl; wherein the monocyclic aryl or the monocyclic heteroaryl is substituted with aryl or heteroaryl. For example, in certain embodiments, B is a monocyclic aryl substituted with an aryl. For example, in certain embodiments, B is a monocyclic aryl substituted with a heteroaryl. For example, in certain embodiments, B is a monocyclic heteroaryl substituted with an aryl. For example, in certain embodiments, B is a monocyclic heteroaryl substituted with a heteroaryl. In certain embodiments, the monocyclic aryl, monocyclic heteroaryl, aryl, or heteroaryl are optionally substituted with one or more substituents selected from the group consisting of alkyl, halo, —OH, and —O-alkyl.

In certain embodiments, B is cyclocyclyl. In certain embodiments, the cyclocyclyl of B ring is optionally substituted with aryl, heteroaryl, —Y^{B}-aryl, or —Y^{B}-heteroaryl, Y^{B} is —O—, —C(O)—, —N(R^{B1})—, —S(O)—, or —S(O)₂—. In certain embodiments, the cyclocyclyl is unsubstituted. In certain embodiments, the cyclocyclyl is substituted with aryl. In certain embodiments, the cyclocyclyl is substituted with heteroaryl. In certain embodiments, the cyclocyclyl is substituted with —Y^{B}-aryl. In certain embodiments, the cyclocyclyl is substituted with —Y^{B}-heteroaryl. In certain embodiments, the cycloalkyl is substituted with —Y^{B}-heterocyclyl. In certain embodiments, the cycloalkyl is substituted with cycloalkyl. In certain embodiments, Y^{B} is —O—. In certain embodiments, Y^{B} is —C(O)—. In certain embodiments, Y^{B} is —N(R^{B1})—. In certain embodiments, Y^{B} is —S(O)—. In certain embodiments, Y^{B} is —S(O)₂—. In certain embodiments, Y^{B} is —CH₂—.

In certain embodiments, B is heterocyclyl. In certain embodiments, the heterocyclyl of B ring is optionally substituted with aryl, heteroaryl, —Y^{B}-aryl, or —Y^{B}— heteroaryl, Y^{B} is —O—, —C(O)—, —N(R^{B1})—, —S(O)—, or —S(O)₂—. In certain embodiments, the heterocyclyl is unsubstituted. In certain embodiments, the heterocyclyl is substituted with aryl. In certain embodiments, the heterocyclyl is substituted with heteroaryl. In certain embodiments, the heterocyclyl is substituted with —Y^{B}-aryl. In certain embodiments, the heterocyclyl is substituted with —Y^{B}-heteroaryl. In certain embodiments, the heterocyclyl is substituted with —Y^{B}-heterocyclyl. In certain embodiments, the heterocyclyl is substituted with cycloalkyl. In certain embodiments, Y^{B} is —O—. In certain embodiments, Y^{B} is —C(O)—. In certain embodiments, Y^{B} is —N(R^{B1})—. In certain embodiments, Y^{B} is —S(O)—. In certain embodiments, Y^{B} is —S(O)₂—. In certain embodiments, Y^{B} is —CH₂—.

As described above for B, the fused bicyclic aryl, the fused bicyclic heteroaryl, —CH₂-aryl, —CH₂-heteroaryl, each aryl, each heteroaryl, cycloalkyl, and —CH₂-heterocyclyl are optionally substituted with one or more substituents selected from the group consisting of alkyl, halo, —CN, —N(R^{B2})₂, —OH, and —O-alkyl; wherein each R^{B2} is independently H or C₁₋₆alkyl.

As described above for B, the fused bicyclic aryl, the fused bicyclic heteroaryl, —CH₂-aryl, —CH₂-heteroaryl, each aryl, each heteroaryl, each cycloalkyl, —CH₂-heterocyclyl, and each heterocyclyl are optionally substituted with one or more substituents selected from the group consisting of alkyl, halo, haloalkyl, —CN, —N(R^{B2})₂, —OH, —O-alkyl, and oxo; wherein each R^{B2} is independently H or C₁₋₆alkyl.

In certain embodiments, B is selected from the group consisting of

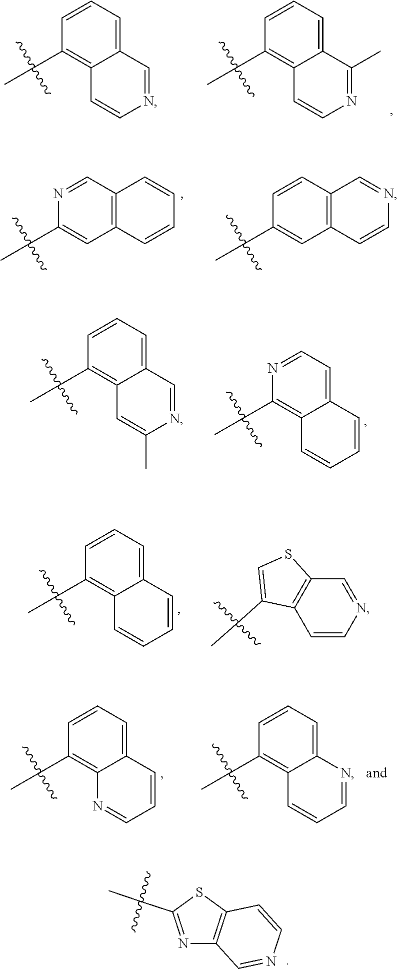

In certain embodiments, B is selected from the group consisting of

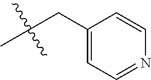 [SMILES: *Cc1ccncc1]

and

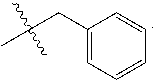 [SMILES: *Cc1ccccc1]

In certain embodiments, B is selected from the group consisting of

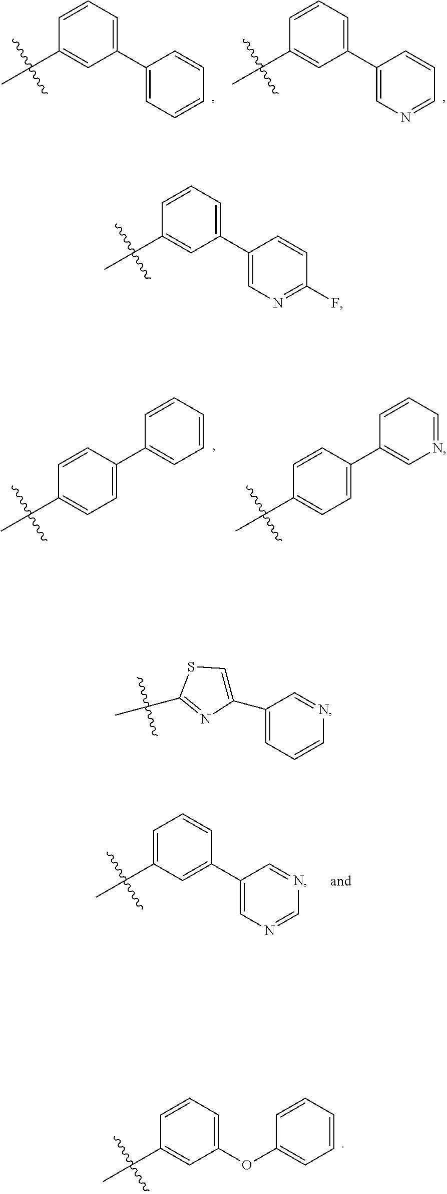

In certain embodiments, B is

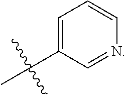 [SMILES: *c1cccnc1]

In certain embodiments, B is

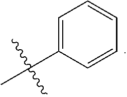 [SMILES: *c1ccccc1]

In certain embodiments, B is

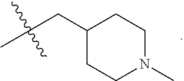 [SMILES: *CC1CCN(C)CC1]

In certain embodiments. B is

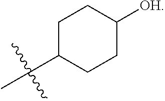 [SMILES: *C1CCC(O)CC1]

In certain embodiments, B is

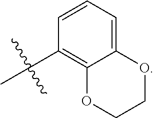 [SMILES: *c1cccc2c1OCCO2]

In certain embodiments, B is

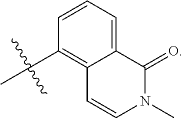 [SMILES: *c1cccc2c(=O)n(C)ccc12]

In certain embodiments, B is

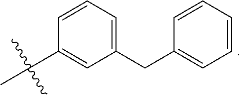 [SMILES: *c1cccc(Cc2ccccc2)c1]

In certain embodiments, B is

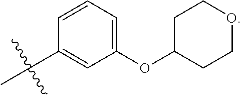 [SMILES: *c1cccc(OC2CCOCC2)c1]

In certain embodiments, B is

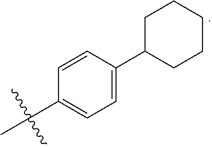 [SMILES: *c1ccc(C2CCCCC2)cc1]

In certain embodiments, B is

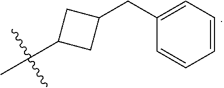 [SMILES: *C1CC(Cc2ccccc2)C1]

In certain embodiments, B is

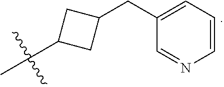 [SMILES: *C1CC(Cc2cccnc2)C1]

In certain embodiments, B is

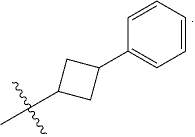 [SMILES: *C1CC(c2ccccc2)C1]

In certain embodiments, B is

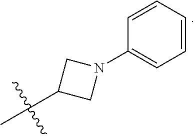 [SMILES: *C1CN(c2ccccc2)C1]

In certain embodiments, B is

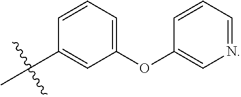 [SMILES: *c1cccc(Oc2cccnc2)c1]

In certain embodiments, B is

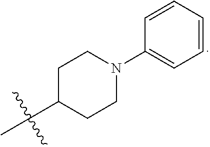 [SMILES: *C1CCN(c2ccccc2)CC1]

In the above, B is optionally substituted.

In some embodiments, the present disclosure provides a compound of formula (I-A), (II-A), (I) or (II) having one, two, or three of the following features:

a) A is aryl;
b) B is a fused bicyclic aryl;
c) L¹ is —C(O)—NR^{L1}—, —O—C(S)—NR^{L1}—, —O—C(O)—NR^{L1}—, or —NR^{L1}—C(S)—NR^{L1}—.

In some embodiments, the present disclosure provides a compound of formula (I-A), (II-A), (I), or (II) having one, two, or three of the following features:

a) A is aryl;
b) B is a fused bicyclic heteroaryl;
c) L¹ is —C(O)—NR^{L1}—, —O—C(S)—NR^{L1}—, —O—C(O)—NR^{L1}— or —NR^{L1}—C(S)—NR^{L1}—.

In some embodiments, the present disclosure provides a compound of formula (I-A), (II-A), (I), or (II) having one, two, or three of the following features:

a) A is aryl;
b) B is aryl substituted with aryl or heteroaryl;
c) L¹ is —C(O)—NR^{L1}—, —O—C(S)—NR^{L1}—, —O—C(O)—NR^{L1}—, or —NR^{L1}—C(S)—NR^{L1}—.

In some embodiments, the present disclosure provides a compound of formula (I-A), (II-A), (I), or (II) having one, two, or three of the following features:

a) A is aryl;
b) B is heteroaryl substituted with aryl or heteroaryl;
c) L¹ is —C(O)—NR^{L1}—, —O—C(S)—NR^{L1}—, —O—C(O)—NR^{L1}—, or —NR^{L1}—C(S)—NR^{L1}—.

In some embodiments, the present disclosure provides a compound of formula (III) having one, two, or three of the following features:

a) A is aryl;
b) B is a fused bicyclic aryl;
c) L³ is —C(O)—NR^{L3}—, —O—C(S)—NR^{L3}—, —O—C(O)—NR^{L3}—, or —NR^{L3}—C(S)—NR^{L3}—.

In some embodiments, the present disclosure provides a compound of formula (III) having one, two, or three of the following features:

a) A is aryl;
b) B is a fused bicyclic heteroaryl;
c) L³ is —C(O)—NR^{L3}—, —O—C(S)—NR^{L3}—, —O—C(O)—NR^{L3}—, or —NR^{L3}—C(S)—NR^{L3}—.

In some embodiments, the present disclosure provides a compound of formula (III) having one, two, or three of the following features:

a) A is aryl;
b) B is aryl substituted with aryl or heteroaryl;
c) L³ is —C(O)—NR^{L3}—, —O—C(S)—NR^{L3}—, —O—C(O)—NR^{L3}—, or —NR^{L3}—C(S)—NR^{L3}—.

In some embodiments, the present disclosure provides a compound of formula (III) having one, two, or three of the following features:

a) A is aryl;
b) B is heteroaryl substituted with aryl or heteroaryl;
c) L³ is —C(O)—NR^{L3}—, —O—C(S)—NR^{L3}—, —O—C(O)—NR^{L3}—, or —NR^{L3}—C(S)—NR^{L3}—.

In some embodiments, the compound of Formula (I-A) or (I) is a compound selected from:

| Compound | |
|---|---|
| No. | Structure |
| I-1 | 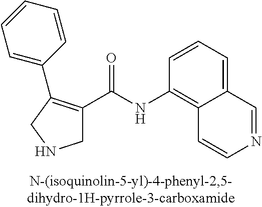 [SMILES: O=C(Nc1cccc2cnccc12)C1=C(c2ccccc2)CNC1] |
| I-2 | 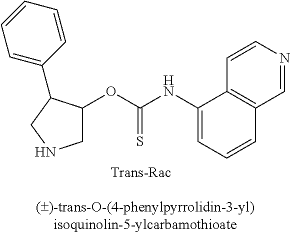 [SMILES: S=C(Nc1cccc2cnccc12)OC1CNCC1c1ccccc1] |
| I-3 | 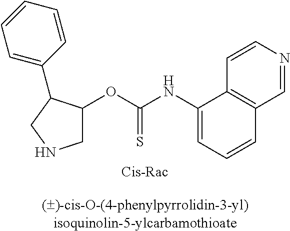 [SMILES: S=C(Nc1cccc2cnccc12)OC1CNCC1c1ccccc1] |
| I-4 | 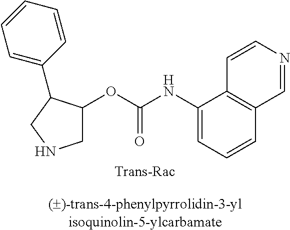 [SMILES: O=C(Nc1cccc2cnccc12)OC1CNCC1c1ccccc1] |
| I-5 | 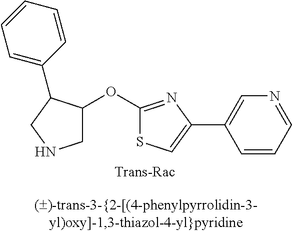 [SMILES: c1ccc(C2CNCC2Oc2nc(-c3cccnc3)cs2)cc1] |
| I-6 | 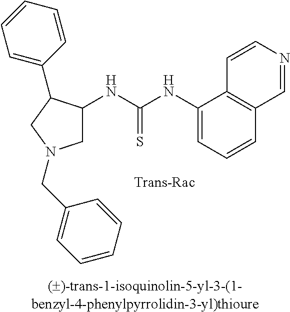 [SMILES: S=C(Nc1cccc2cnccc12)NC1CN(Cc2ccccc2)CC1c1ccccc1] |
| I-7 | 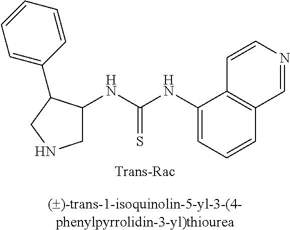 [SMILES: S=C(Nc1cccc2cnccc12)NC1CNCC1c1ccccc1] |
| I-8 | 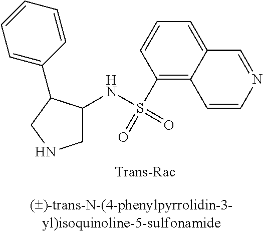 [SMILES: O=S(=O)(NC1CNCC1c1ccccc1)c1cccc2cnccc12] |
| I-9 | 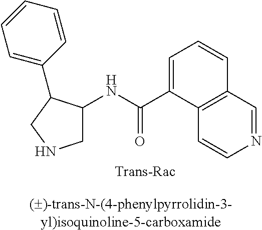 [SMILES: O=C(NC1CNCC1c1ccccc1)c1cccc2cnccc12] |
| I-10 | 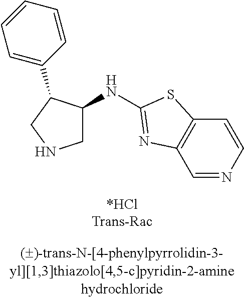 [SMILES: c1ccc([C@H]2CNC[C@@H]2Nc2nc3cnccc3s2)cc1] |
| I-11 | 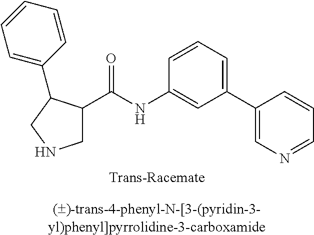 [SMILES: O=C(Nc1cccc(-c2cccnc2)c1)C1CNCC1c1ccccc1] |
| I-12 | 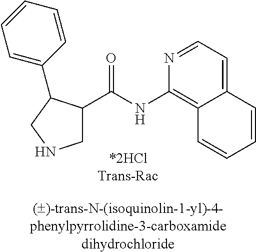 [SMILES: O=C(Nc1nccc2ccccc12)C1CNCC1c1ccccc1] |
| I-13 | 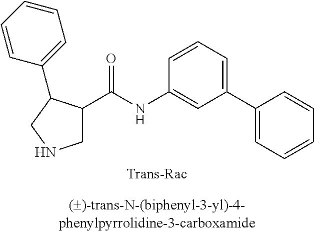 [SMILES: O=C(Nc1cccc(-c2ccccc2)c1)C1CNCC1c1ccccc1] |
| I-14 | 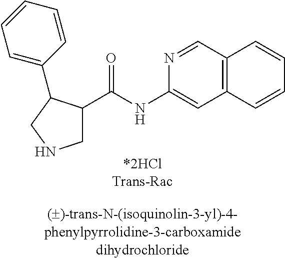 [SMILES: O=C(Nc1cc2ccccc2cn1)C1CNCC1c1ccccc1] |
| I-15 | 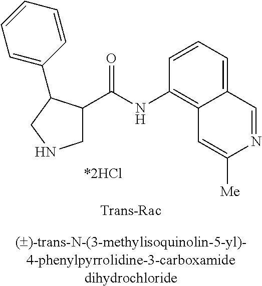 [SMILES: Cc1cc2c(NC(=O)C3CNCC3c3ccccc3)cccc2cn1] |
| I-16 | 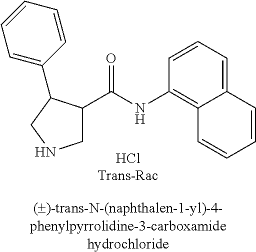 [SMILES: O=C(Nc1cccc2ccccc12)C1CNCC1c1ccccc1] |
| I-17 | 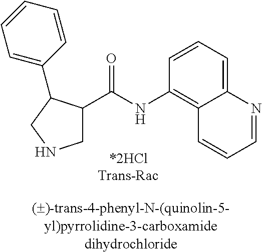 [SMILES: O=C(Nc1cccc2ncccc12)C1CNCC1c1ccccc1] |
| I-18 | 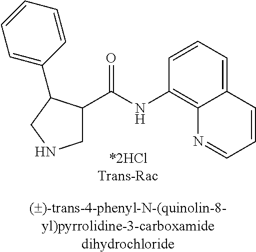 [SMILES: O=C(Nc1cccc2cccnc12)C1CNCC1c1ccccc1] |
| I-19 | 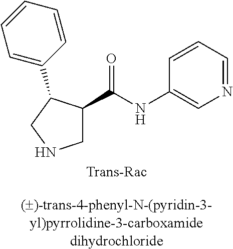 [SMILES: O=C(Nc1cccnc1)[C@H]1CNC[C@@H]1c1ccccc1] |
| I-20 | 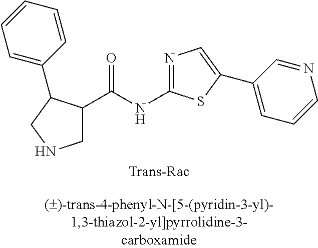 [SMILES: O=C(Nc1ncc(-c2cccnc2)s1)C1CNCC1c1ccccc1] |
| I-21 | 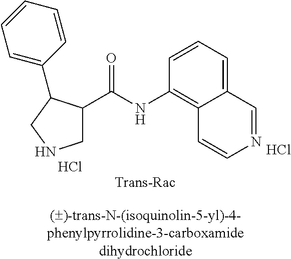 [SMILES: O=C(Nc1cccc2cnccc12)C1CNCC1c1ccccc1 ; Cl ; Cl] |
| I-22 | 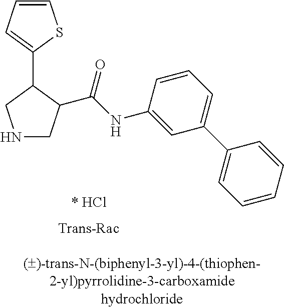 [SMILES: O=C(Nc1cccc(-c2ccccc2)c1)C1CNCC1c1cccs1] |
| I-23 | 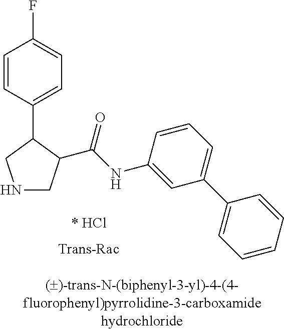 [SMILES: O=C(Nc1cccc(-c2ccccc2)c1)C1CNCC1c1ccc(F)cc1] |
| I-24 | 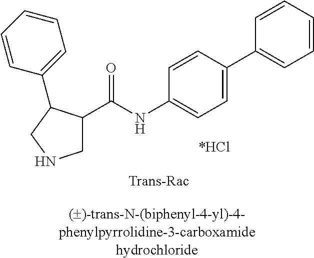 [SMILES: O=C(Nc1ccc(-c2ccccc2)cc1)C1CNCC1c1ccccc1] |
| I-25 | 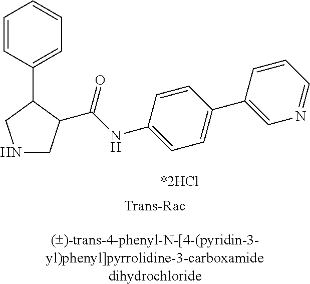 [SMILES: O=C(Nc1ccc(-c2cccnc2)cc1)C1CNCC1c1ccccc1] |
| I-26 | 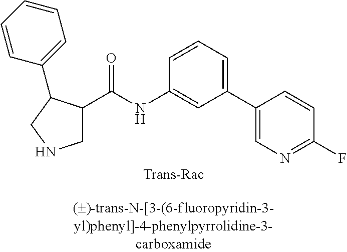 [SMILES: O=C(Nc1cccc(-c2ccc(F)nc2)c1)C1CNCC1c1ccccc1] |
| I-27 | 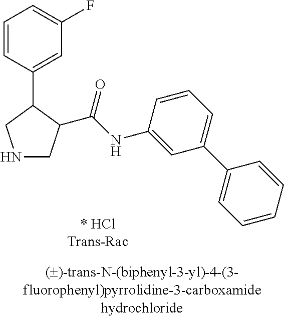 [SMILES: O=C(Nc1cccc(-c2ccccc2)c1)C1CNCC1c1cccc(F)c1] |
| I-28 | 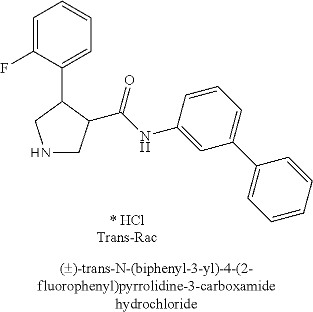 [SMILES: O=C(Nc1cccc(-c2ccccc2)c1)C1CNCC1c1ccccc1F] |
| I-29 | 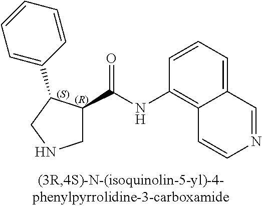 |
| I-30 | 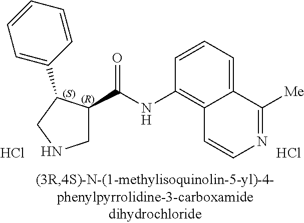 |
| I-31 | 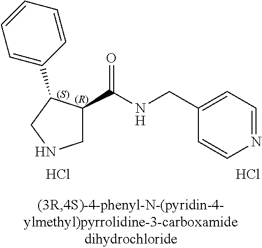 |
| I-32 | 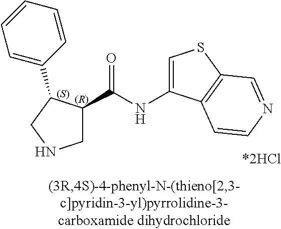 |
| I-33 | 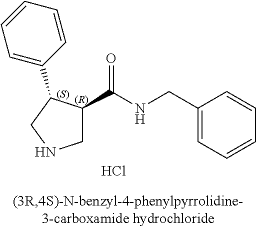 |
| I-34 | 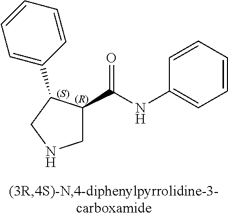 |
| I-35 | 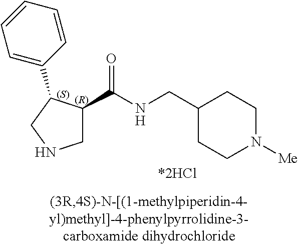 |
| I-36 | 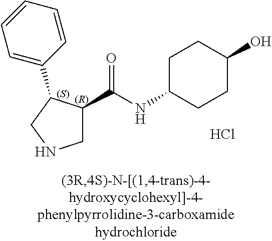 |
| I-37 | 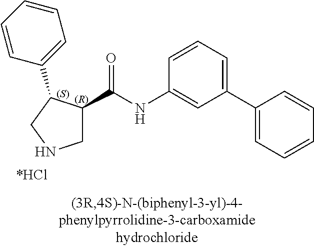 |
| I-38 | 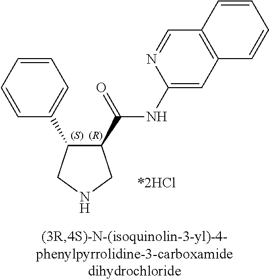 |
| I-39 | 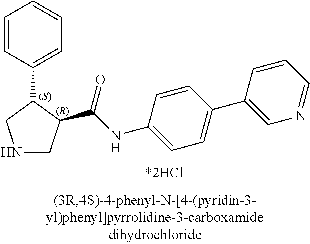 |
| I-40 | 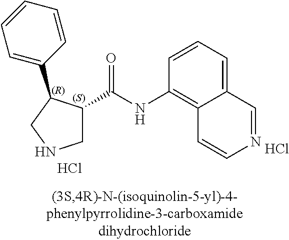 |
| I-41 | 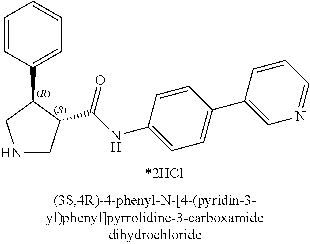 |
| I-42 | 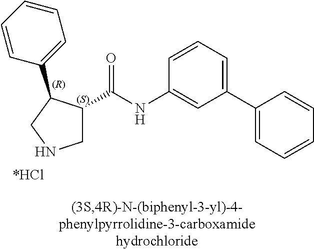 |
| I-43 | 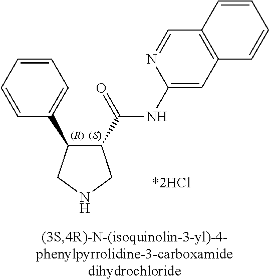 |
| I-44 | 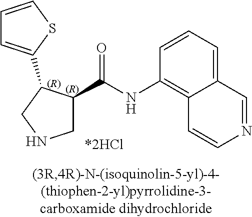 |
| I-45 | 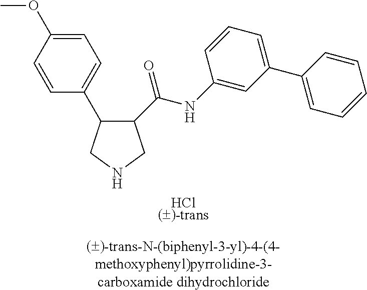 |
| I-46 | 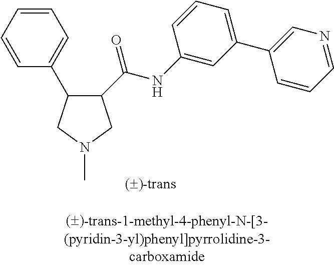 |
| I-47 | 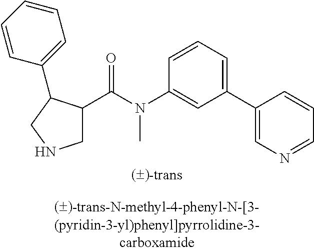 |
| I-48 | 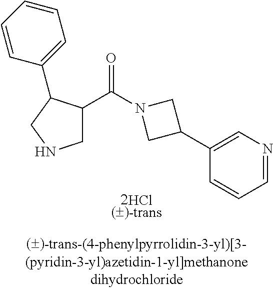 |
| I-49 | 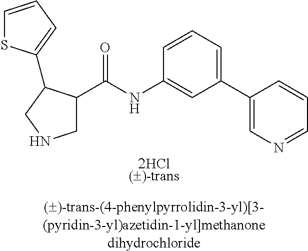 |
| I-50 | 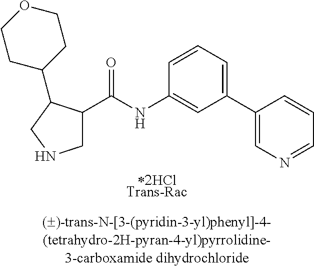 [SMILES: O=C(Nc1cccc(-c2cccnc2)c1)C1CNCC1C1CCOCC1 ; "*2HClTrans-Rac"] |
| I-51 | 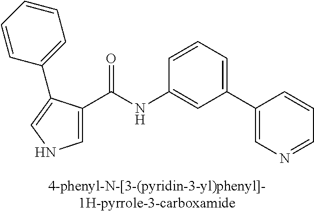 [SMILES: O=C(Nc1cccc(-c2cccnc2)c1)c1c[nH]cc1-c1ccccc1] |
| I-52 | 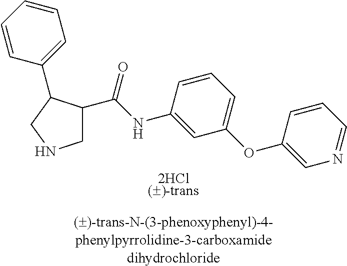 |
| I-53 | 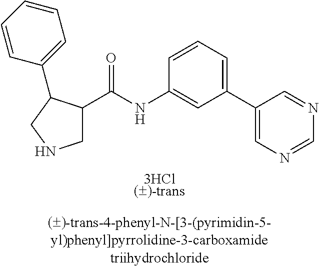 |
| I-54 | 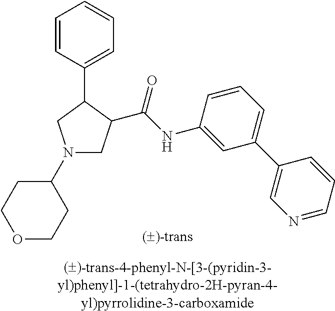 |
| I-55 | 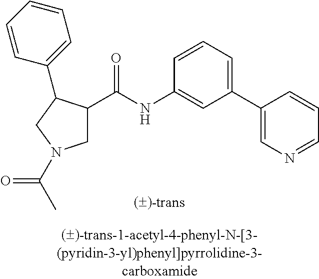 |
| I-56 | 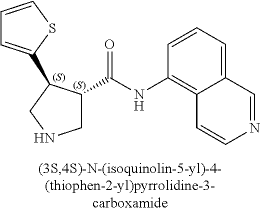 |
| I-57 | 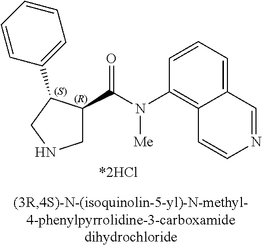 |
| I-58 | 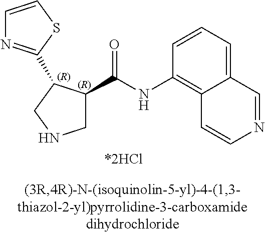 |
| I-59 | 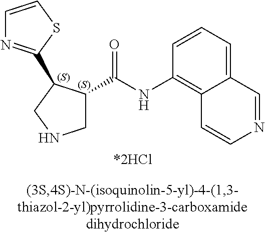 |

and pharmaceutically acceptable salts and tautomers thereof.

In some embodiments, the compound of Formula (I-A) or (I) is a compound selected from:

| Compound | |
|---|---|
| No. | Structure |
| I-60 | 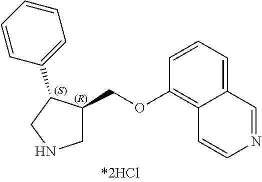 |
| I-61 | 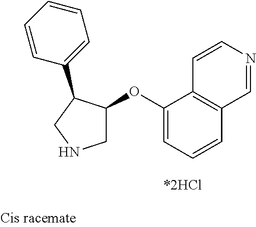 [SMILES: c1ccc([C@@H]2CNC[C@@H]2Oc2cccc3cnccc23)cc1] |
| I-62 | 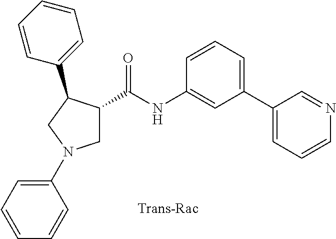 [SMILES: O=C(Nc1cccc(-c2cccnc2)c1)[C@@H]1CN(c2ccccc2)C[C@H]1c1ccccc1] |
| I-63 | 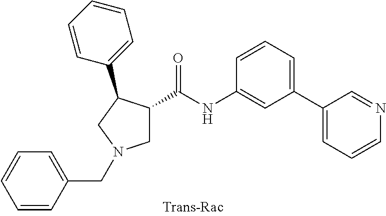 [SMILES: O=C(Nc1cccc(-c2cccnc2)c1)[C@@H]1CN(Cc2ccccc2)C[C@H]1c1ccccc1] |
| I-64 | 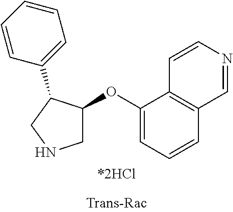 [SMILES: c1ccc([C@H]2CNC[C@@H]2Oc2cccc3cnccc23)cc1] |
| I-65 | 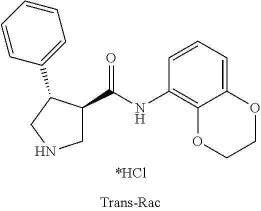 [SMILES: O=C(Nc1cccc2c1OCCO2)[C@H]1CNC[C@@H]1c1ccccc1] |
| I-66 | 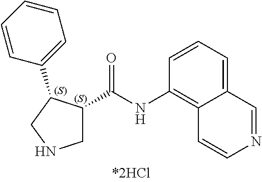 |
| I-67 | 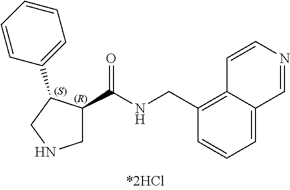 |
| I-68 | 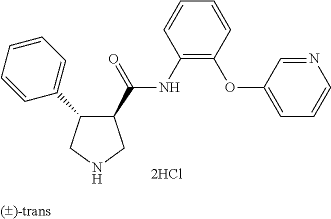 [SMILES: O=C(Nc1ccccc1Oc1cccnc1)[C@H]1CNC[C@@H]1c1ccccc1] |
| I-69 | 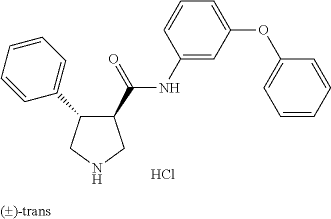 [SMILES: O=C(Nc1cccc(Oc2ccccc2)c1)[C@H]1CNC[C@@H]1c1ccccc1 ; Cl] |
| I-70 | 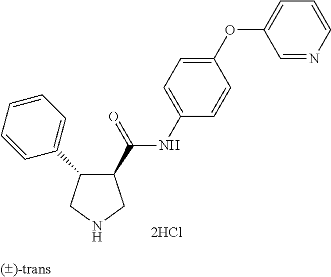 [SMILES: O=C(Nc1ccc(Oc2cccnc2)cc1)[C@H]1CNC[C@@H]1c1ccccc1] |
| I-71 | 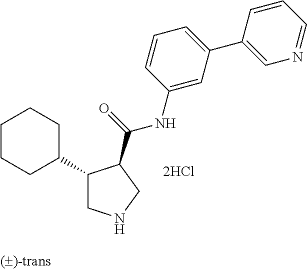 [SMILES: O=C(Nc1cccc(-c2cccnc2)c1)[C@H]1CNC[C@@H]1C1CCCCC1] |
| I-72 | 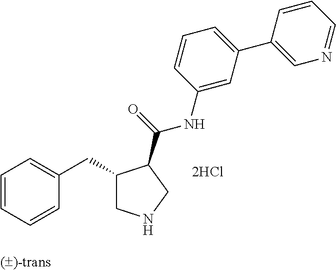 [SMILES: O=C(Nc1cccc(-c2cccnc2)c1)[C@H]1CNC[C@@H]1Cc1ccccc1] |
| I-73 | 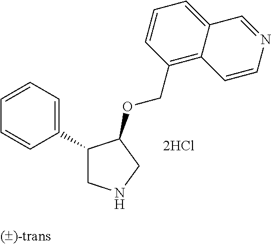 [SMILES: c1ccc([C@H]2CNC[C@@H]2OCc2cccc3cnccc23)cc1] |
| I-74 | 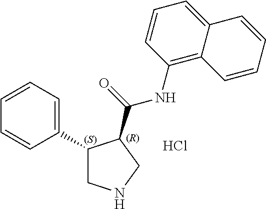 |
| I-75 | 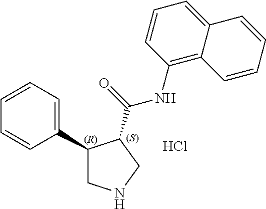 |
| I-76 | 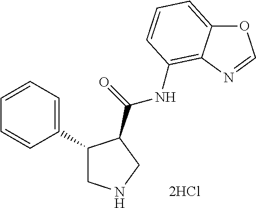 [SMILES: O=C(Nc1cccc2ocnc12)[C@H]1CNC[C@@H]1c1ccccc1 ; "2HCl"] |

and pharmaceutically acceptable salts and tautomers thereof.

In some embodiments, the compound of Formula (II-A) or (II) is a compound selected from:

| Compound | |
|---|---|
| No. | Structure |
| I-77 | 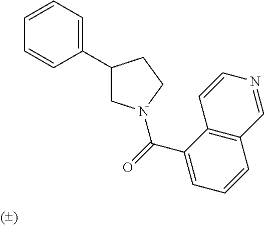 [SMILES: O=C(c1cccc2cnccc12)N1CCC(c2ccccc2)C1] |
| I-78 | 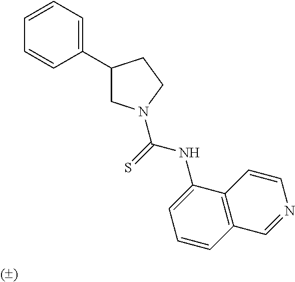 [SMILES: S=C(Nc1cccc2cnccc12)N1CCC(c2ccccc2)C1] |
| I-79 | 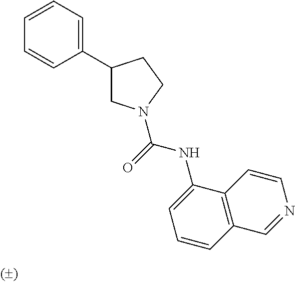 [SMILES: O=C(Nc1cccc2cnccc12)N1CCC(c2ccccc2)C1] |
| I-80 | 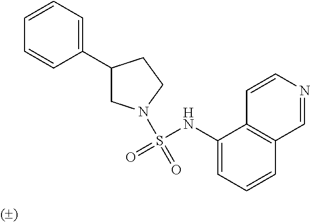 [SMILES: O=S(=O)(Nc1cccc2cnccc12)N1CCC(c2ccccc2)C1] |

and pharmaceutically acceptable salts and tautomers thereof.

In some embodiments, the compound of Formula (I-A) or (I) is a compound selected from:

| Compound | |
|---|---|
| No. | Structure |
| I-81 | 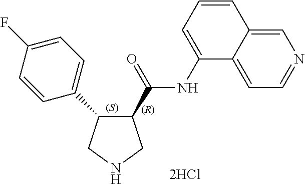 |
| I-82 | 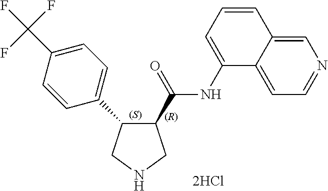 |
| I-83 | 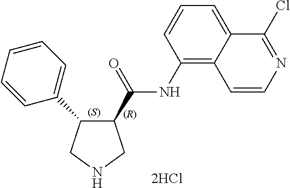 |
| I-84 | 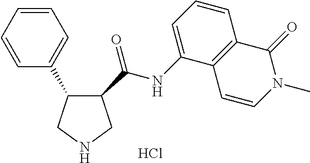 [SMILES: Cn1ccc2c(NC(=O)[C@H]3CNC[C@@H]3c3ccccc3)cccc2c1=O ; Cl] |
| I-85 | 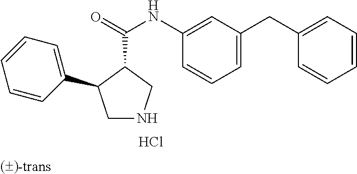 |
| I-86 | 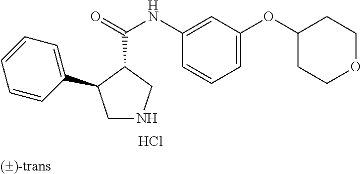 |
| I-87 | 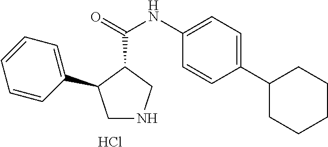 [SMILES: O=C(Nc1ccc(C2CCCCC2)cc1)[C@@H]1CNC[C@H]1c1ccccc1 ; Cl] |
| I-88 | 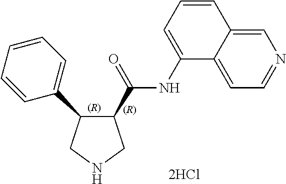 |
| I-89 | 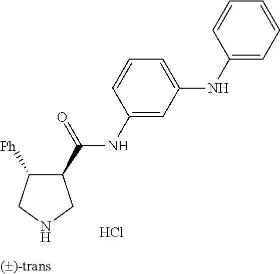 [SMILES: O=C(Nc1cccc(Nc2ccccc2)c1)[C@H]1CNC[C@@H]1c1ccccc1 ; Cl] |
| I-90 | 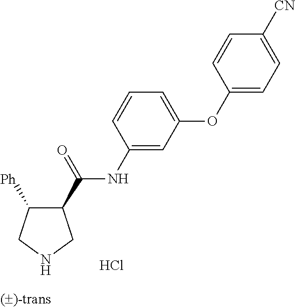 [SMILES: N#Cc1ccc(Oc2cccc(NC(=O)[C@H]3CNC[C@@H]3c3ccccc3)c2)cc1 ; Cl] |
| I-91 | 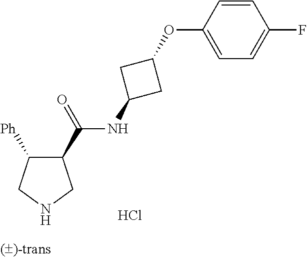 [SMILES: O=C(N[C@H]1C[C@H](Oc2ccc(F)cc2)C1)[C@H]1CNC[C@@H]1c1ccccc1 ; Cl] |
| I-92 | 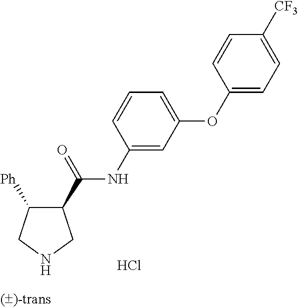 [SMILES: O=C(Nc1cccc(Oc2ccc(C(F)(F)F)cc2)c1)[C@H]1CNC[C@@H]1c1ccccc1 ; Cl] |
| I-93 | 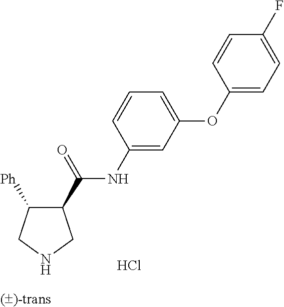 [SMILES: O=C(Nc1cccc(Oc2ccc(F)cc2)c1)[C@H]1CNC[C@@H]1c1ccccc1 ; Cl] |
| I-94 | 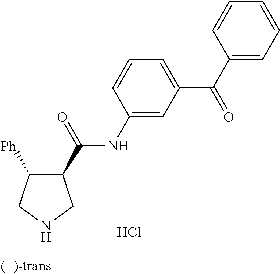 [SMILES: O=C(c1ccccc1)c1cccc(NC(=O)[C@H]2CNC[C@@H]2c2ccccc2)c1 ; Cl] |
| I-95 | 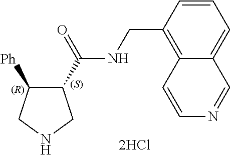 |
| I-96 | 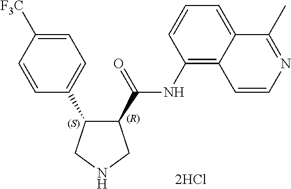 |
| I-97 | 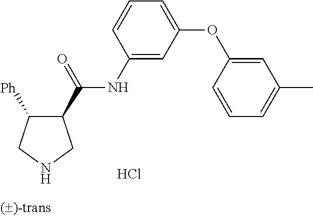 [SMILES: Cc1cccc(Oc2cccc(NC(=O)[C@H]3CNC[C@@H]3c3ccccc3)c2)c1 ; Cl] |
| I-98 | 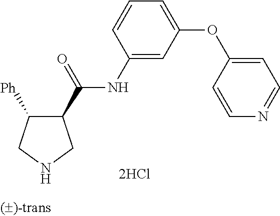 [SMILES: O=C(Nc1cccc(Oc2ccncc2)c1)[C@H]1CNC[C@@H]1c1ccccc1 ; "2HCl"] |
| I-99 | 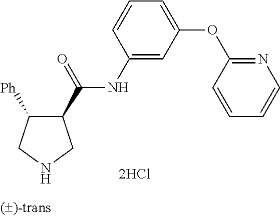 [SMILES: O=C(Nc1cccc(Oc2ccccn2)c1)[C@H]1CNC[C@@H]1c1ccccc1 ; "2HCl"] |
| I-100 | 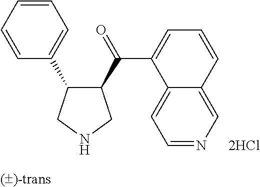 [SMILES: O=C(c1cccc2cnccc12)[C@H]1CNC[C@@H]1c1ccccc1 ; "2HCl"] |
| I-101 | 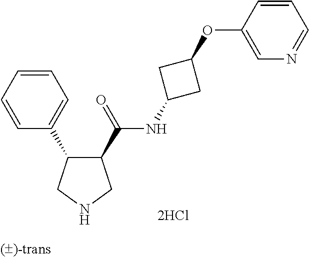 [SMILES: O=C(N[C@H]1C[C@H](Oc2cccnc2)C1)[C@H]1CNC[C@@H]1c1ccccc1 ; "2HCl"] |
| I-102 | 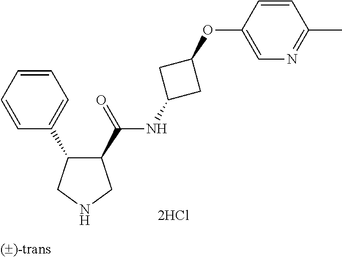 [SMILES: Cc1ccc(O[C@H]2C[C@H](NC(=O)[C@H]3CNC[C@@H]3c3ccccc3)C2)cn1 ; "2HCl"] |
| I-103 | 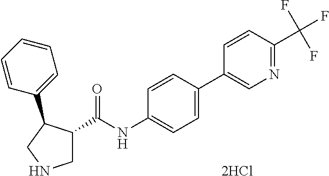 [SMILES: O=C(Nc1ccc(-c2ccc(C(F)(F)F)nc2)cc1)[C@@H]1CNC[C@H]1c1ccccc1 ; "2HCl"] |
| I-104 | 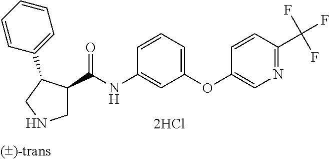 |
| I-105 | 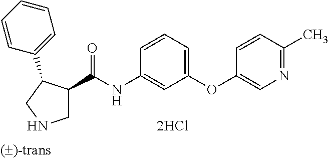 |
| I-106 | 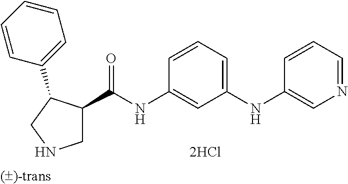 |
| I-107 | 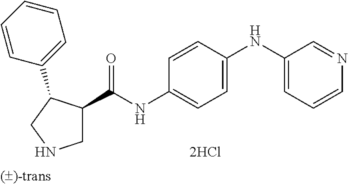 |
| I-108 | 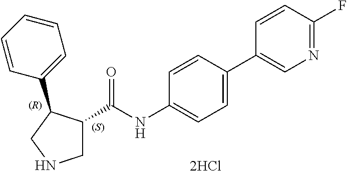 |
| I-109 | 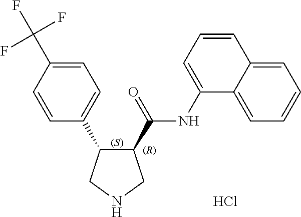 |
| I-110 | 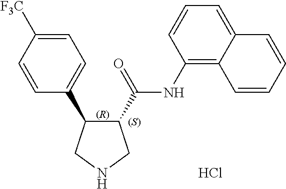 |
| I-111 | 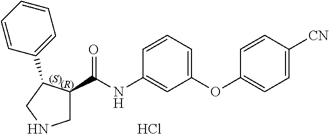 |
| I-112 | 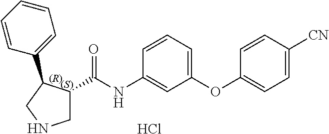 |
| I-113 | 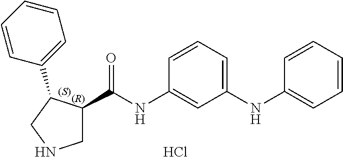 |
| I-114 | 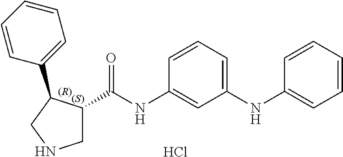 |
| I-115 | 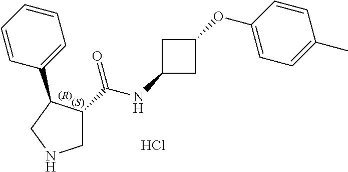 |
| I-116 | 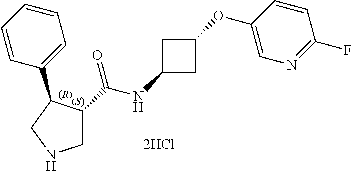 |
| I-117 | 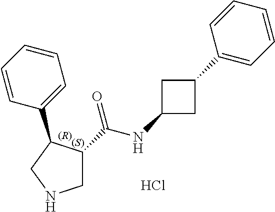 |
| I-118 | 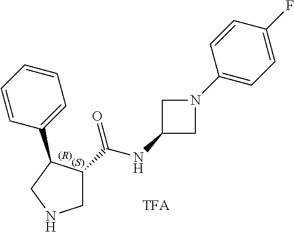 |
| I-119 | 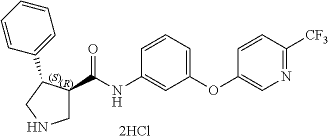 |
| I-120 | 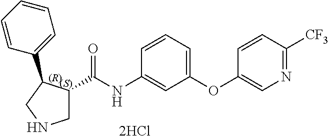 |
| I-121 | 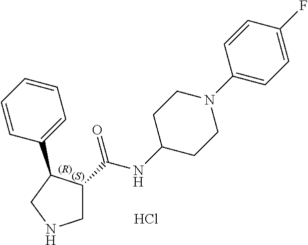 |
| I-122 | 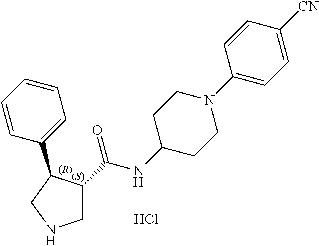 |
| I-123 | 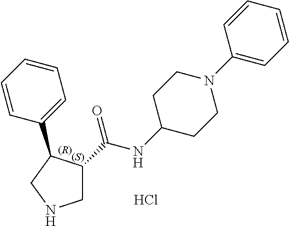 |

and pharmaceutically acceptable salts and tautomers thereof.

In some embodiments, the compound of Formula (I-A) or (I) is a compound selected from:

| | |
|---|---|
| I- 124 | 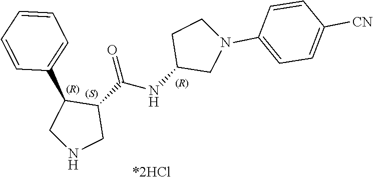 |
| I- 125 | 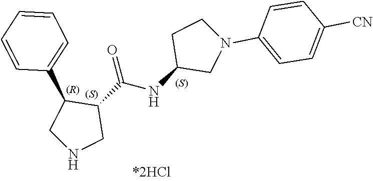 |
| I- 126 | 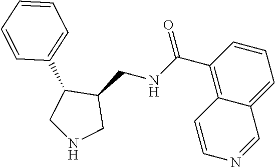 [SMILES: O=C(NC[C@H]1CNC[C@@H]1c1ccccc1)c1cccc2cnccc12] |
| I- 127 | 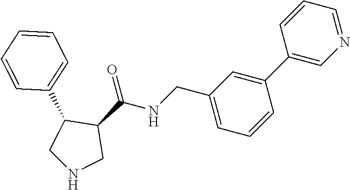 [SMILES: O=C(NCc1cccc(-c2cccnc2)c1)[C@H]1CNC[C@@H]1c1ccccc1] |
| I- 128 | 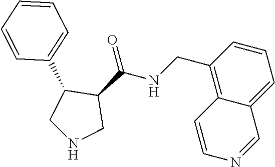 [SMILES: O=C(NCc1cccc2cnccc12)[C@H]1CNC[C@@H]1c1ccccc1] |
| I- 129 | 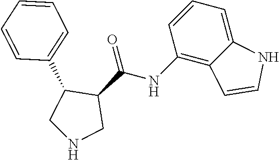 [SMILES: O=C(Nc1cccc2[nH]ccc12)[C@H]1CNC[C@@H]1c1ccccc1] |
| I- 130 | 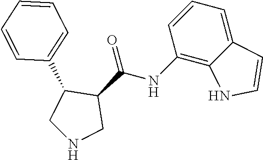 [SMILES: O=C(Nc1cccc2cc[nH]c12)[C@H]1CNC[C@@H]1c1ccccc1] |
| I- 131 | 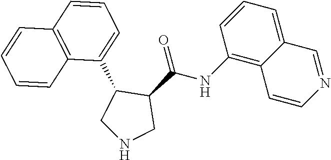 [SMILES: O=C(Nc1cccc2cnccc12)[C@H]1CNC[C@@H]1c1cccc2ccccc12] |
| I- 132 | 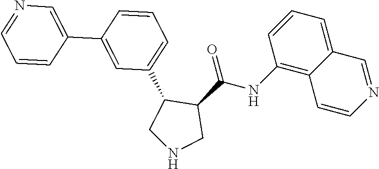 [SMILES: O=C(Nc1cccc2cnccc12)[C@H]1CNC[C@@H]1c1cccc(-c2cccnc2)c1] |
| I- 133 | 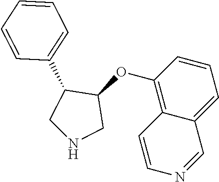 [SMILES: c1ccc([C@H]2CNC[C@@H]2Oc2cccc3cnccc23)cc1] |
| I- 134 | 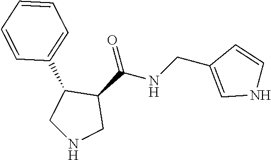 [SMILES: O=C(NCc1cc[nH]c1)[C@H]1CNC[C@@H]1c1ccccc1] |
| I- 135 | 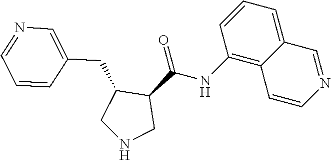 [SMILES: O=C(Nc1cccc2cnccc12)[C@H]1CNC[C@@H]1Cc1cccnc1] |
| I- 136 | 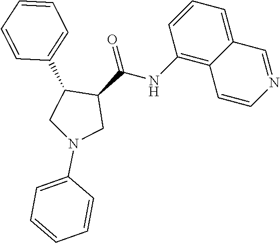 [SMILES: O=C(Nc1cccc2cnccc12)[C@H]1CN(c2ccccc2)C[C@@H]1c1ccccc1] |
| I- 137 | 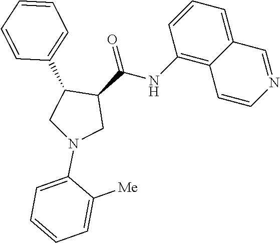 [SMILES: Cc1ccccc1N1C[C@H](C(=O)Nc2cccc3cnccc23)[C@@H](c2ccccc2)C1] |
| I- 138 | 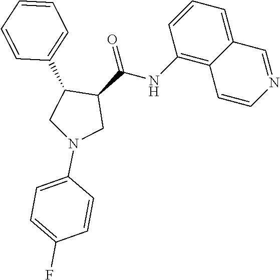 [SMILES: O=C(Nc1cccc2cnccc12)[C@H]1CN(c2ccc(F)cc2)C[C@@H]1c1ccccc1] |
| I- 139 | 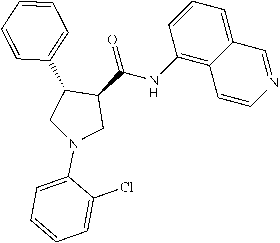 [SMILES: O=C(Nc1cccc2cnccc12)[C@H]1CN(c2ccccc2Cl)C[C@@H]1c1ccccc1] |

and pharmaceutically acceptable salts and tautomers thereof.

In some embodiments, the compound of Formula (II-A) or (II) is a compound selected from:

| | |
|---|---|
| I-140 | 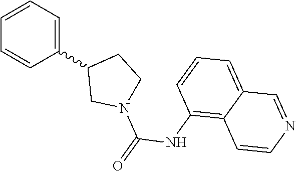 [SMILES: O=C(Nc1cccc2cnccc12)N1CCC(c2ccccc2)C1] |
| I-141 | 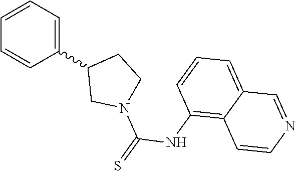 [SMILES: S=C(Nc1cccc2cnccc12)N1CCC(c2ccccc2)C1] |
| I-142 | 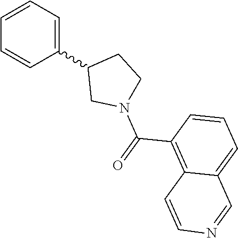 [SMILES: O=C(c1cccc2cnccc12)N1CCC(c2ccccc2)C1] |
| I-143 | 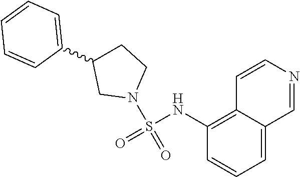 [SMILES: O=S(=O)(Nc1cccc2cnccc12)N1CCC(c2ccccc2)C1] |
| I-144 | 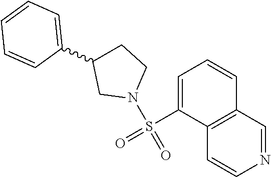 [SMILES: O=S(=O)(c1cccc2cnccc12)N1CCC(c2ccccc2)C1] |
| I-145 | 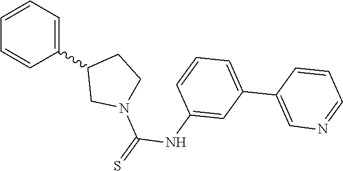 [SMILES: S=C(Nc1cccc(-c2cccnc2)c1)N1CCC(c2ccccc2)C1] |
| I-146 | 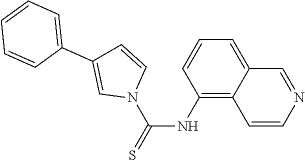 [SMILES: S=C(Nc1cccc2cnccc12)n1ccc(-c2ccccc2)c1] |
| I-147 | 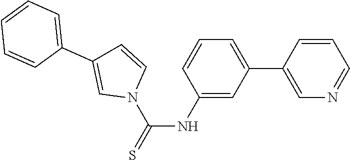 [SMILES: S=C(Nc1cccc(-c2cccnc2)c1)n1ccc(-c2ccccc2)c1] |
| I-148 | 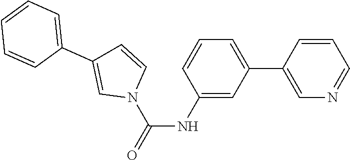 [SMILES: O=C(Nc1cccc(-c2cccnc2)c1)n1ccc(-c2ccccc2)c1] |
| I-149 | 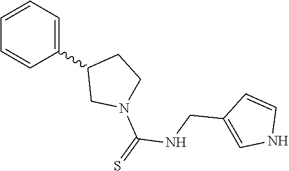 [SMILES: S=C(NCc1cc[nH]c1)N1CCC(c2ccccc2)C1] |
| I-150 | 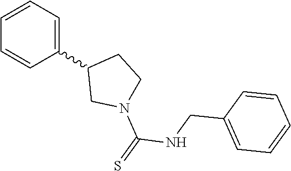 [SMILES: S=C(NCc1ccccc1)N1CCC(c2ccccc2)C1] |
| I-151 | 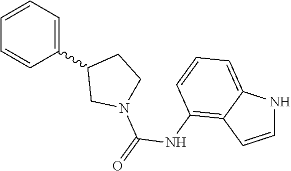 [SMILES: O=C(Nc1cccc2[nH]ccc12)N1CCC(c2ccccc2)C1] |
| I-152 | 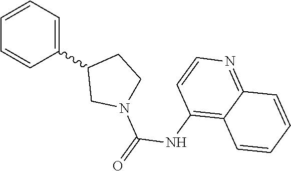 [SMILES: O=C(Nc1ccnc2ccccc12)N1CCC(c2ccccc2)C1] |
| I-153 | 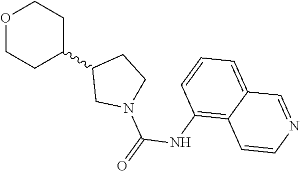 [SMILES: O=C(Nc1cccc2cnccc12)N1CCC(C2CCOCC2)C1] |
| I-154 | 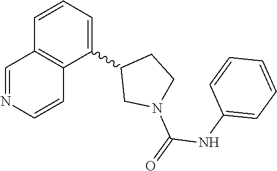 [SMILES: O=C(Nc1ccccc1)N1CCC(c2cccc3cnccc23)C1] |
| I-155 | 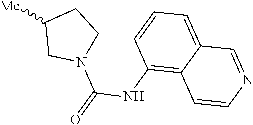 [SMILES: CC1CCN(C(=O)Nc2cccc3cnccc23)C1] |
| I-156 | 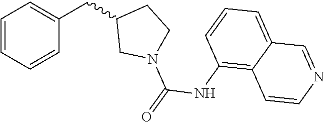 [SMILES: O=C(Nc1cccc2cnccc12)N1CCC(Cc2ccccc2)C1] |
| I-157 | 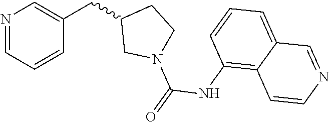 [SMILES: O=C(Nc1cccc2cnccc12)N1CCC(Cc2cccnc2)C1] |
| I-158 | 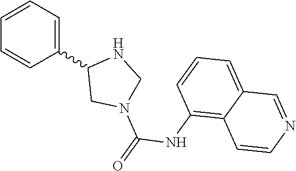 [SMILES: O=C(Nc1cccc2cnccc12)N1CNC(c2ccccc2)C1] |
| I-159 | 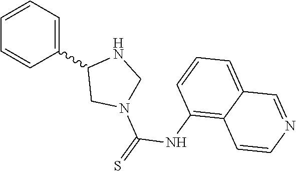 [SMILES: S=C(Nc1cccc2cnccc12)N1CNC(c2ccccc2)C1] |
| I-160 | 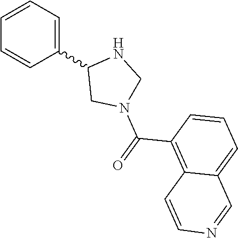 [SMILES: O=C(c1cccc2cnccc12)N1CNC(c2ccccc2)C1] |
| I-161 | 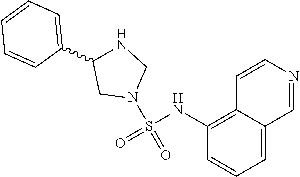 [SMILES: O=S(=O)(Nc1cccc2cnccc12)N1CNC(c2ccccc2)C1] |
| I-162 | 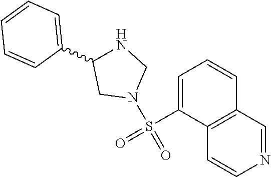 [SMILES: O=S(=O)(c1cccc2cnccc12)N1CNC(c2ccccc2)C1] |
| I-163 | 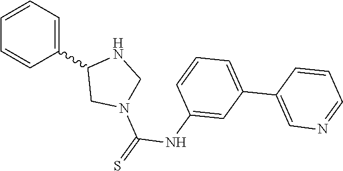 [SMILES: S=C(Nc1cccc(-c2cccnc2)c1)N1CNC(c2ccccc2)C1] |
| I-164 | 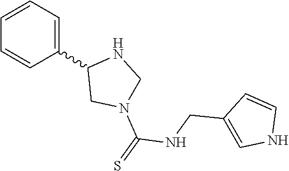 [SMILES: S=C(NCc1cc[nH]c1)N1CNC(c2ccccc2)C1] |
| I-165 | 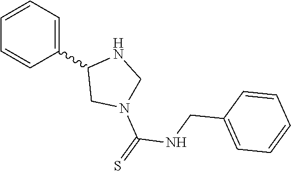 [SMILES: S=C(NCc1ccccc1)N1CNC(c2ccccc2)C1] |
| I-166 | 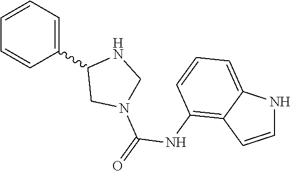 [SMILES: O=C(Nc1cccc2[nH]ccc12)N1CNC(c2ccccc2)C1] |
| I-167 | 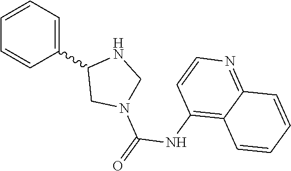 [SMILES: O=C(Nc1ccnc2ccccc12)N1CNC(c2ccccc2)C1] |
| I-168 | 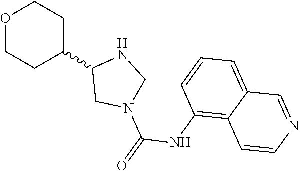 [SMILES: O=C(Nc1cccc2cnccc12)N1CNC(C2CCOCC2)C1] |
| I-169 | 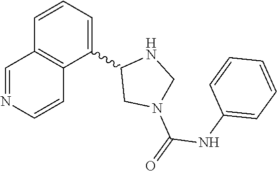 [SMILES: O=C(Nc1ccccc1)N1CNC(c2cccc3cnccc23)C1] |
| I-170 | 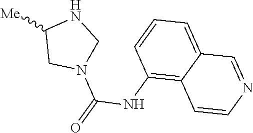 [SMILES: CC1CN(C(=O)Nc2cccc3cnccc23)CN1] |
| I-171 | 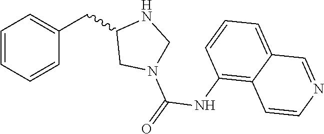 [SMILES: O=C(Nc1cccc2cnccc12)N1CNC(Cc2ccccc2)C1] |

and pharmaceutically acceptable salts and tautomers thereof.

It should be understood, that such references are intended to encompass not only the above general formula, but also each and every of the embodiments, etc. discussed in the following. It should also be understood, that unless stated to the opposite, such references also encompass isomers, mixtures of isomers, pharmaceutically acceptable salts, solvates and prodrugs of the compounds of Formula (I-A), (II-A), (I), (II), or (III).

### Methods for the Preparation of Compounds

The compounds of the present disclosure (e.g., compounds of Formula (I)) can be prepared in a number of ways well known to those skilled in the art of organic synthesis. By way of example, compounds of the present disclosure can be synthesized using the methods described below, together with synthetic methods known in the art of synthetic organic chemistry, or variations thereon as appreciated by those skilled in the art. Preferred methods include but are not limited to those methods described below. The final products of the reactions described herein may be isolated by conventional techniques, e.g., by extraction, crystallisation, distillation, chromatography, etc.

Compounds of the present disclosure can be synthesized by following the steps outlined in General Schemes 1-3. Starting materials are either commercially available or made by known procedures in the reported literature or as illustrated. Useful steps that may be used in the preparation steps of the compounds will be known to the skilled person. The method below is given as a non-limiting example on how the compounds may be prepared.

### General Scheme 1. General Method for the Preparation of Racemates Compounds

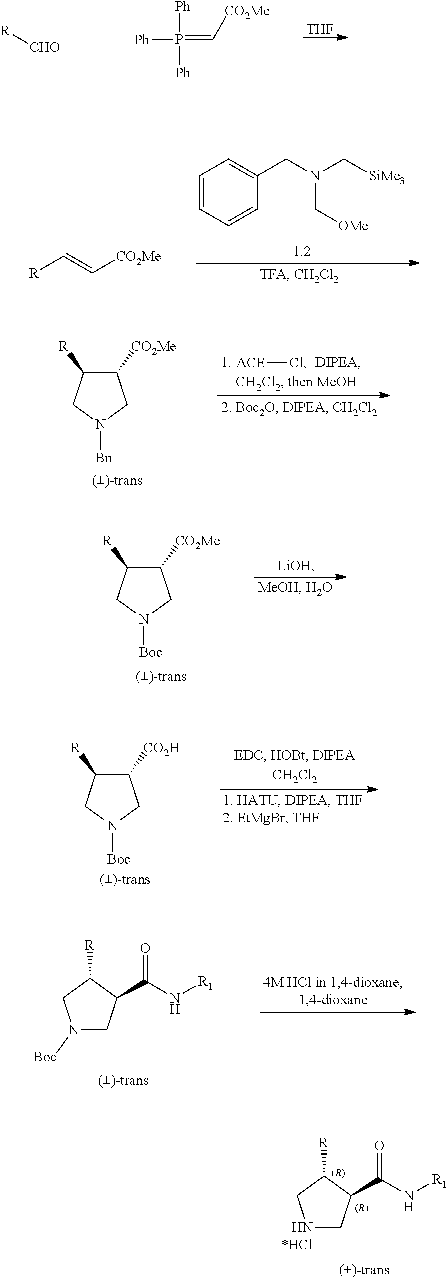

General Scheme 2. General Method for the Preparation of Compounds with 3R,4S Absolute Configuration

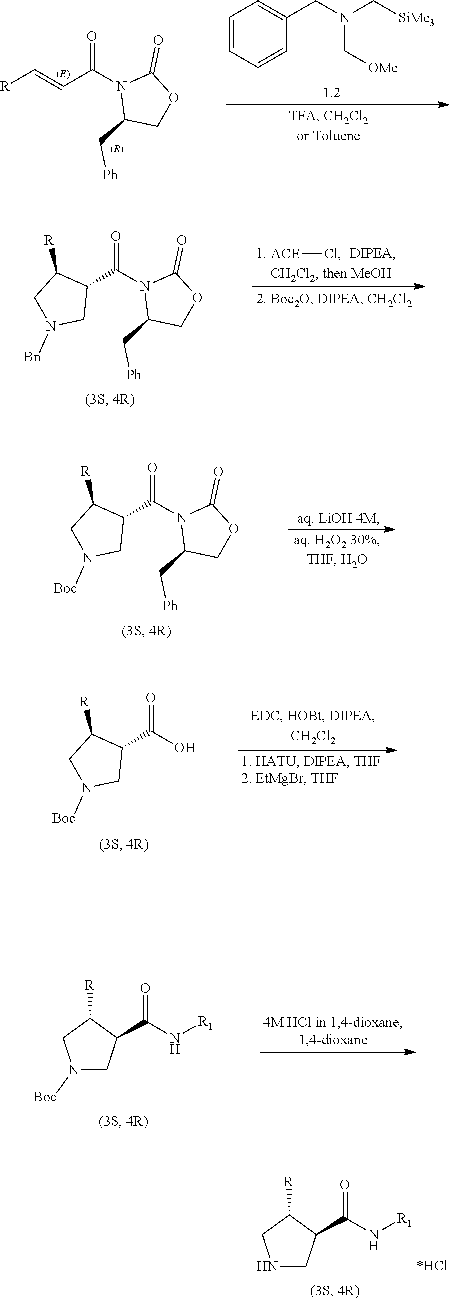

General Scheme 3. General Method for the Preparation of Compounds with 3S,4R Absolute Configuration

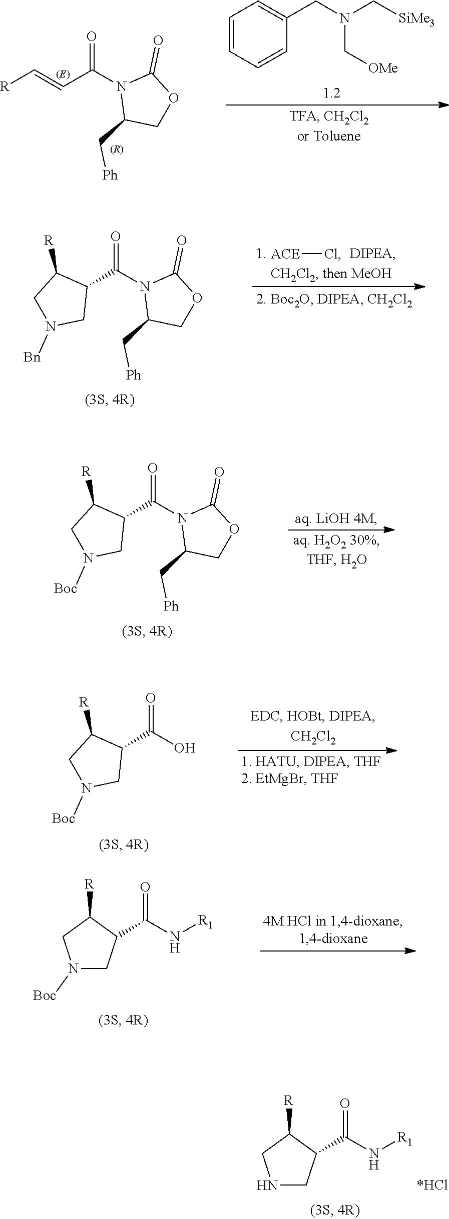

A mixture of enantiomers, diastereomers, cis/trans isomers resulting from the process described above can be separated into their single components by chiral salt technique, chromatography using normal phase, reverse phase or chiral column, depending on the nature of the separation.

It should be understood that in the description and formula shown above, the various groups A ring, B ring, X, R¹, L¹, L², and other variables are as defined herein above, except where otherwise indicated. Furthermore, for synthetic purposes, the compounds of General Schemes 1-3 are merely representative with elected radicals to illustrate the general synthetic methodology of the disclosed compounds.

### Pharmaceutical Compositions

The compound of Formula (I-A), (II-A), (I), (II), or (III) may be provided in any form suitable for the intended administration, in particular including pharmaceutically acceptable salts, solvates and prodrugs of the compound of Formula (I-A), (II-A), (I), (II), or (III).

Pharmaceutically acceptable salts refer to salts of the compounds of Formula (I-A), (II-A), (I), (II), or (III) which are considered to be acceptable for clinical and/or veterinary use. Typical pharmaceutically acceptable salts include those salts prepared by reaction of the compounds of Formula (I-A), (II-A), (I), (II), or (III) and a mineral or organic acid or an organic or inorganic base. Such salts are known as acid addition salts and base addition salts, respectively. It will be recognized that the particular counter-ion forming a part of any salt is not of a critical nature, so long as the salt as a whole is pharmaceutically acceptable and as long as the counter-ion does not contribute undesired qualities to the salt as a whole. These salts may be prepared by methods known to the skilled person. Pharmaceutically acceptable salts are, e.g., those described and discussed in Remington's Pharmaceutical Sciences, 17. Ed. Alfonso R. Gennaro (Ed.), Mack Publishing Company, Easton, Pa., U.S.A., 1985 and more recent editions and in Encyclopedia of Pharmaceutical Technology.

Examples of pharmaceutically acceptable addition salts include acid addition salts formed with inorganic acids, e.g., hydrochloric, hydrobromic, sulfuric, nitric, hydroiodic, metaphosphoric, or phosphoric acid; and organic acids e.g., succinic, maleic, acetic, fumaric, citric, tartaric, benzoic, trifluoroacetic, malic, lactic, formic, propionic, glycolic, gluconic, camphorsulfuric, isothionic, mucic, gentisic, isonicotinic, saccharic, glucuronic, furoic, glutamic, ascorbic, anthranilic, salicylic, phenylacetic, mandelic, embonic (pamoic), ethanesulfonic, pantothenic, stearic, sulfinilic, alginic, and galacturonic acid; and arylsulfonic, for example benzenesulfonic, p-toluenesulfonic, methanesulfonic, or naphthalenesulfonic acid; and base addition salts formed with alkali metals and alkaline earth metals and organic bases such as N,N-dibenzylethylenediamine, chloroprocaine, choline, diethanolamine, ethylenediamine, meglumine (N-methylglucamine), lysine, and procaine; and internally formed salts. It should be understood that all references to pharmaceutically acceptable salts include solvent addition forms (solvates) or crystal forms (polymorphs) as defined herein, of the same salt.

The compound of Formula (I-A), (II-A), (I), (II), or (III), or a pharmaceutically acceptable salt thereof, may be provided in dissoluble or indissoluble forms together with a pharmaceutically acceptable solvent such as water, ethanol, and the like. Dissoluble forms may also include hydrated forms such as the mono-hydrate, the dihydrate, the hemihydrate, the trihydrate, the tetrahydrate, and the like.

The compound of Formula (I-A), (II-A), (I), (II), or (III), or a pharmaceutically acceptable salt thereof, may be provided as a prodrug. The term “prodrug” used herein is intended to mean a compound which upon exposure to certain physiological conditions—will liberate the compound of Formula (I-A), (II-A), (I), (II), or (III), or a pharmaceutically acceptable salt thereof, which then will be able to exhibit the desired biological action. A typical example is a labile carbamate of an amine.

Since prodrugs are known to enhance numerous desirable qualities of pharmaceuticals (e.g., solubility, bioavailability, manufacturing, etc.), the compounds of the present disclosure can be delivered in prodrug form. Thus, the present disclosure is intended to cover prodrugs of the presently claimed compounds, methods of delivering the same and compositions containing the same. “Prodrugs” are intended to include any covalently bonded carriers that release an active parent drug of the present disclosure in vivo when such prodrug is administered to a subject. Prodrugs in the present disclosure are prepared by modifying functional groups present in the compound in such a way that the modifications are cleaved, either in routine manipulation or in vivo, to the parent compound. Prodrugs include compounds of the present disclosure wherein a hydroxy, amino, sulfhydryl, carboxy, or carbonyl group is bonded to any group that may be cleaved in vivo to form a free hydroxyl, free amino, free sulfhydryl, free carboxy, or free carbonyl group, respectively.

Examples of prodrugs include, but are not limited to, esters (e.g., acetate, dialkylaminoacetates, formates, phosphates, sulfates, and benzoate derivatives) and carbamates (e.g., N,N-dimethylaminocarbonyl) of hydroxy functional groups, esters (e.g., C₁₋₆ alkyl esters, e.g., methyl esters, ethyl esters, 2-propyl esters, phenyl esters, 2-aminoethyl esters, morpholinoethanol esters, etc.) of carboxyl functional groups, N-acyl derivatives (e.g., N-acetyl), N-Mannich bases, Schiff bases, and enaminones of amino functional groups, oximes, acetals, ketals, and enol esters of ketone and aldehyde functional groups in compounds of the disclosure, and the like. See Bundegaard, H., Design of Prodrugs, p1-92, Elesevier, New York-Oxford (1985).

The compounds, or pharmaceutically acceptable salts, esters or prodrugs thereof, are administered orally, nasally, transdermally, pulmonary, inhalationally, buccally, sublingually, intraperintoneally, subcutaneously, intramuscularly, intravenously, rectally, intrapleurally, intrathecally, and parenterally. In one embodiment, the compound is administered orally. One skilled in the art will recognize the advantages of certain routes of administration.

The dosage regimen utilizing the compounds is selected in accordance with a variety of factors including type, species, age, weight, sex, and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter or arrest the progress of the condition.

Techniques for formulation and administration of the disclosed compounds of the disclosure can be found in Remington: the Science and Practice of Pharmacy, 19^{th} edition, Mack Publishing Co., Easton, Pa. (1995). In an embodiment, the compounds described herein, and the pharmaceutically acceptable salts thereof, are used in pharmaceutical preparations in combination with a pharmaceutically acceptable carrier or diluent. Suitable pharmaceutically acceptable carriers include inert solid fillers or diluents and sterile aqueous or organic solutions. The compounds will be present in such pharmaceutical compositions in amounts sufficient to provide the desired dosage amount in the range described herein.

In one aspect of this disclosure, there is provided a pharmaceutical composition comprising at, as an active ingredient, at least one compound of Formula (I-A), (II-A), (I), (II), or (III), or a pharmaceutically acceptable salt thereof, as defined herein, and optionally one or more pharmaceutically acceptable excipients, diluents and/or carriers. The compounds of Formula (I-A), (II-A), (I), (II), or (III), or a pharmaceutically acceptable salt thereof, may be administered alone or in combination with pharmaceutically acceptable carriers, diluents or excipients, in either single or multiple doses. Suitable pharmaceutically acceptable carriers, diluents and excipients include inert solid diluents or fillers, sterile aqueous solutions, and various organic solvents.

A “pharmaceutical composition” is a formulation containing the compounds of the present disclosure in a form suitable for administration to a subject. The pharmaceutical compositions may be formulated with pharmaceutically acceptable carriers or diluents as well as any other known adjuvants and excipients in accordance with conventional techniques such as those disclosed in Remington: The Science and Practice of Pharmacy, 21st Edition, 2000, Lippincott Williams & Wilkins.

As used herein, the phrase “pharmaceutically acceptable” refers to those compounds, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

The pharmaceutical compositions formed by combining a compound of Formula (I-A), (II-A), (I), (II), or (III), or a pharmaceutically acceptable salt thereof, as defined herein, with pharmaceutically acceptable carriers, diluents or excipients can be readily administered in a variety of dosage forms such as tablets, powders, lozenges, syrups, suppositories, injectable solutions, and the like. In powders, the carrier is a finely divided solid such as talc or starch which is in a mixture with the finely divided active component. In tablets, the active component is mixed with the carrier having the necessary binding properties in suitable proportions and compacted in the shape and size desired.

The pharmaceutical compositions may be specifically prepared for administration by any suitable route such as the oral and parenteral (including subcutaneous, intramuscular, intrathecal, intravenous and intradermal) route. It will be appreciated that the preferred route will depend on the general condition and age of the subject to be treated, the nature of the condition to be treated and the active ingredient chosen.

Pharmaceutical compositions for oral administration include solid dosage forms such as capsules, tablets, dragees, pills, lozenges, powders, and granules. Where appropriate, they can be prepared with coatings such as enteric coatings or they can be prepared so as to provide controlled release of the active ingredient such as sustained or prolonged release according to methods well known in the art.

For oral administration in the form of a tablet or capsule, a compound of Formula (I-A), (II-A), (I), (II), or (III), or a pharmaceutically acceptable salt thereof, as defined herein, may suitably be combined with an oral, non-toxic, pharmaceutically acceptable carrier such as ethanol, glycerol, water, or the like. Furthermore, suitable binders, lubricants, disintegrating agents, flavoring agents, and colourants may be added to the mixture, as appropriate. Suitable binders include, e.g., lactose, glucose, starch, gelatin, acacia gum, tragacanth gum, sodium alginate, carboxymethylcellulose, polyethylene glycol, waxes, or the like. Lubricants include, e.g., sodium oleate, sodium stearate, magnesium stearate, sodium benzoate, sodium acetate, sodium chloride, or the like. Disintegrating agents include, e.g., starch, methyl cellulose, agar, bentonite, xanthan gum, sodium starch glycolate, crospovidone, croscarmellose sodium, or the like. Additional excipients for capsules include macrogels or lipids.

For the preparation of solid compositions such as tablets, the active compound of Formula (I-A), (II-A), (I), (II), or (III), or a pharmaceutically acceptable salt thereof, is mixed with one or more excipients, such as the ones described above, and other pharmaceutical diluents such as water to make a solid pre-formulation composition containing a homogenous mixture of a compound of Formula (I-A), (II-A), (I), (II), or (III), or a pharmaceutically acceptable salt thereof. The term “homogenous” is understood to mean that the compound of Formula (I), (II), or (III), or a pharmaceutically acceptable salt thereof, is dispersed evenly throughout the composition so that the composition may readily be subdivided into equally effective unit dosage forms such as tablets or capsules.

Liquid compositions for either oral or parenteral administration of the compound of Formula (I-A), (II-A), (I), (II), or (III), or a pharmaceutically acceptable salt thereof, include, e.g., aqueous solutions, syrups, elixirs, aqueous or oil suspensions, and emulsion with edible oils such as cottonseed oil, sesame oil, coconut oil, or peanut oil. Suitable dispersing or suspending agents for aqueous suspensions include synthetic or natural gums such as tragacanth, alginate, acacia, dextran, sodium carboxymethylcellulose, gelatin, methylcellulose, or polyvinylpyrrolidone.

Pharmaceutical compositions for parenteral administration include sterile aqueous and non-aqueous injectable solutions, dispersions, suspensions or emulsions as well as sterile powders to be reconstituted in sterile injectable solutions or dispersions prior to use.

For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, and sodium chloride in the composition. Prolonged absorption of the injectable compositions can be brought about by including in the composition an agent which delays absorption, for example, aluminum monostearate and gelatin.

The preparation of all these solutions under sterile conditions is readily accomplished by standard pharmaceutical techniques well known to those skilled in the art.

For example, sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof. Depot injectable compositions are also contemplated as being within the scope of the present disclosure.

For parenteral administration, solutions containing a compound of Formula (I-A), (II-A), (I), (II), or (III), or a pharmaceutically acceptable salt thereof, in sesame or peanut oil, aqueous propylene glycol, or in sterile aqueous solution may be employed. Such aqueous solutions should be suitably buffered if necessary and the liquid diluent first rendered isotonic with sufficient saline or glucose. These particular aqueous solutions are especially suitable for intravenous, intramuscular, subcutaneous, and intraperitoneal administration. The oily solutions are suitable for intra-articular, intra-muscular, and subcutaneous injection purposes.

In addition to the aforementioned ingredients, the compositions of a compound of Formula (I-A), (II-A), (I), (II), or (III), or a pharmaceutically acceptable salt thereof, may include one or more additional ingredients such as diluents, buffers, flavouring agents, colourant, surface active agents, thickeners, preservatives, e.g., methyl hydroxybenzoate (including anti-oxidants), emulsifying agents, and the like.

The term “therapeutically effective amount”, as used herein, refers to an amount of a pharmaceutical agent to treat, ameliorate, or prevent an identified disease, disorder, or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician. In a preferred aspect, the disease or disorder to be treated is a disease or disorder associated with modulation of NR2F6.

For any compound, the therapeutically effective amount can be estimated initially either in cell culture assays, e.g., in cells, or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred. The dosage may vary within this range depending upon the dosage form employed, sensitivity of the patient, and the route of administration.

Dosage and administration are adjusted to provide sufficient levels of the active agent(s) or to maintain the desired effect. Factors which may be taken into account include the severity of the disease state, general health of the subject, age, weight, and gender of the subject, diet, time, and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

A suitable dosage of the compound of Formula (I-A), (II-A), (I), (II), or (III), or a pharmaceutically acceptable salt thereof, will depend on the age and condition of the patient, the severity of the disease to be treated and other factors well known to the practicing physician. The compound may be administered for example either orally, parenterally, or topically according to different dosing schedules, e.g., daily or with intervals, such as weekly intervals. In general a single dose will be in the range from 0.01 to 500 mg/kg body weight, preferably from about 0.05 to 100 mg/kg body weight, more preferably between 0.1 to 50 mg/kg body weight, and most preferably between 0.1 to 25 mg/kg body weight. The compound may be administered as a bolus (i.e., the entire daily dose is administered at once) or in divided doses two or more times a day. Variations based on the aforementioned dosage ranges may be made by a physician of ordinary skill taking into account known considerations such as weight, age, and condition of the person being treated, the severity of the affliction, and the particular route of administration.

The compounds of Formula (I-A), (II-A), (I), (II), or (III), or a pharmaceutically acceptable salt thereof, may also be prepared in a pharmaceutical composition comprising one or more further active substances alone, or in combination with pharmaceutically acceptable carriers, diluents, or excipients in either single or multiple doses.

### Methods of Treatment

The present disclosure provides a method of modulating activity of NR2F6 by exposure of NR2F6 to an effective amount of a compound of Formula (I-A), (II-A), (I), (II), or (III), or a pharmaceutically acceptable salt or tautomer thereof, or a pharmaceutical composition comprising a compound of Formula (I-A), (II-A), (I), (II), or (III), or a pharmaceutically acceptable salt or tautomer thereof. The present disclosure provides a method of treating or reducing the effect of a disease or disorder associated with NR2F6 modulation, the method comprising administration of an effective amount of a compound of Formula (I-A), (II-A), (I), (II), or (III), or a pharmaceutically acceptable salt or tautomer thereof, or the pharmaceutical composition comprising a compound of Formula (I-A), (II-A), (I), (II), or (III), or a pharmaceutically acceptable salt or tautomer thereof.

The present disclosure provides a compound of Formula (I-A), (II-A), (I), (II), or (III), or a pharmaceutically acceptable salt or tautomer thereof, or a pharmaceutical composition comprising a compound of Formula (I-A), (II-A), (I), (II), or (III), or a pharmaceutically acceptable salt or tautomer thereof for use in modulating activity of NR2F6 by exposure of NR2F6. The present disclosure provides a compound of Formula (I-A), (II-A), (I), (II), or (III), or a pharmaceutically acceptable salt or tautomer thereof, or a pharmaceutical composition comprising a compound of Formula (I-A), (II-A), (I), (II), or (III), or a pharmaceutically acceptable salt or tautomer thereof for use in treating or reducing the effect of a disease or disorder associated with NR2F6 modulation.

The present disclosure provides use of a compound of Formula (I-A), (II-A), (I), (II), or (III), or a pharmaceutically acceptable salt or tautomer thereof, or a pharmaceutical composition comprising a compound of Formula (I-A), (II-A), (I), (II), or (III), or a pharmaceutically acceptable salt or tautomer thereof for modulating activity of NR2F6 by exposure of NR2F6. The present disclosure provides use of a compound of Formula (I-A), (II-A), (I), (II), or (III), or a pharmaceutically acceptable salt or tautomer thereof, or a pharmaceutical composition comprising a compound of Formula (I-A), (II-A), (I), (II), or (III), or a pharmaceutically acceptable salt or tautomer thereof for treating or reducing the effect of a disease or disorder associated with NR2F6 modulation.

The present disclosure provides use of a compound of Formula (I-A), (II-A), (I), (II), or (III), or a pharmaceutically acceptable salt or tautomer thereof, or a pharmaceutical composition comprising a compound of Formula (I-A), (II-A), (I), (II), or (III), or a pharmaceutically acceptable salt or tautomer thereof, in the manufacture of a medicament for modulating activity of NR2F6. The present disclosure provides use of a compound of Formula (I-A), (II-A), (I), (II), or (III), or a pharmaceutically acceptable salt or tautomer thereof, or a pharmaceutical composition comprising a compound of Formula (I-A), (II-A), (I), (II), or (III), or a pharmaceutically acceptable salt or tautomer thereof, in the manufacture of a medicament for treating or reducing the effect of a disease or disorder associated with NR2F6 modulation.

Disclosed are compounds useful for modulation of NR2F6 activity. In some embodiments, compounds disclosed are utilized for stimulation of NR2F6 activity. In some embodiments, the present disclosure provides for use of compounds for inhibition of NR2F6 activation. Stimulation of NR2F6 within the context of the present disclosure is useful, intra alia, for induction of immune inhibition, or stimulation of cellular proliferation without significant induction of differentiation. Inhibition of NR2F6 is desired in situations where the skilled artisan seeks to augment immune response, or induce cellular differentiation. In some embodiments, inhibition of NR2F6 expression is desired in situations where inhibition of cancer or cancer stem cells is needed.

In certain embodiments, the modulation comprises augmentation of NR2F6 activity. In certain embodiments, the modulation comprises inhibition of NR2F6 activity.

Accordingly, the present disclosure provides compounds that bind to NR2F6 molecules or to portion of NR2F6, which as are at least 60%, 70%, 80%, 90%, 95%, 96%, 97%, 98%, or 99% identical to the amino acid sequence of NR2F6.

The term “agonist” or “activator” as used herein is known in the art and relates to a compound/substance capable of fully or partially stimulating the physiologic activity of (a) specific receptor(s). In the context of the present disclosure, an agonist, therefore, may stimulate the physiological activity of a receptor such as NR2F6 upon binding of said compound/substance to said receptor. Binding of an “agonist/activator” to a given receptor, e.g. NR2F6, may mimic the action of an endogenous ligand binding to said receptor. As used herein, accordingly, the term “agonist” also encompasses partial agonists or co-agonists/co-activators. In addition thereto, however, an “agonist” or “activator” of NR2F6 in the context of the present disclosure may also be capable of stimulating the function of a given receptor, such as NR2F6, by inducing/enhancing the expression of the nucleic acid molecule encoding for said receptor. Thus, an agonist/activator of NR2F6 may lead to an increased expression level of NR2F6 (e.g. increased level of NR2F6 mRNA, NR2F6 protein) which is reflected in an increased activity of NR2F6. An activator of NR2F6 in the context of the present disclosure, accordingly, may also encompass transcriptional activators of NR2F6 expression that are capable of enhancing NR2F6 function. The term “agonist” comprises partial agonists. As partial agonists, the art defines candidate molecules that behave like agonists, but that, even at high concentrations, cannot activate NR2F6 to the same extend as a full agonist. An increased expression and/or activity of NR2F6 by an agonist/activator of NR2F6 leads to a decreased activity (and/or expression) of components of the NR2F6-dependent signaling pathway; in particular the activity of NF-AT and AP-1 is decreased. NF-AT/AP-1 regulate transcription/expression of further “downstream” components of the NR2F6-dependent signaling pathway, such as IL-2, IL-17, and/or IFN-gamma. A decrease in NF-AT/AP-1 activity results in a decreased transcription of these “downstream” components (e.g. IL-2, IL-17, and/or IFN-gamma) which in turn leads to a suppression of an immune response. In sum, the herein described agonist/activator of NR2F6 will, accordingly, lead to a suppression of an immune response. Hence, the use of potent agonists/activators of NR2F6 will lead to a higher expression and/or activity of NR2F6.

An increase of NR2F6 activity leads to a decreased activity of NF-AT/AP-1 (and other components of the NR2F6-dependent signalling pathway) which in turn results in a suppressed immune response. Therefore, agonists/activators of NR2F6 can be useful in the treatment of diseases where suppression of the immune response is desired (e.g. diseases with an overstimulated immune response, such as allergies and multiple sclerosis).

In certain embodiments, the disorder is cancer. An inhibition of NR2F6 according to the present disclosure can be used for immunotherapies for treating cancer. “Treating a cancer”, “inhibiting cancer”, “reducing cancer growth” refers to inhibiting or preventing oncogenic activity of cancer cells. Oncogenic activity can comprise inhibiting migration, invasion, drug resistance, cell survival, anchorage-independent growth, non-responsiveness to cell death signals, angiogenesis, or combinations thereof of the cancer cells. The terms “cancer”, “cancer cell”, “tumor”, and “tumor cell” are used interchangeably herein and refer generally to a group of diseases characterized by uncontrolled, abnormal growth of cells (e.g., a neoplasia). In some forms of cancer, the cancer cells can spread locally or through the bloodstream and lymphatic system to other parts of the body (“metastatic cancer”). “Ex vivo activated lymphocytes”, “lymphocytes with enhanced antitumor activity”, and “dendritic cell cytokine induced killers” are terms used interchangeably to refer to composition of cells that have been activated ex vivo and subsequently reintroduced within the context of the present disclosure. Although the word “lymphocyte” is used, this also includes heterogenous cells that have been expanded during the ex vivo culturing process including dendritic cells, NKT cells, gamma delta T cells, and various other innate and adaptive immune cells. As used herein, “cancer” refers to all types of cancer or neoplasm or malignant tumors found in animals, including leukemias, carcinomas and sarcomas. Examples of cancers are cancer of the brain, melanoma, bladder, breast, cervix, colon, head and neck, kidney, lung, non-small cell lung, mesothelioma, ovary, prostate, sarcoma, stomach, uterus, and medulloblastoma.

The term “leukemia” is meant broadly progressive, malignant diseases of the hematopoietic organs/systems and is generally characterized by a distorted proliferation and development of leukocytes and their precursors in the blood and bone marrow. Leukemia diseases include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophilic leukemia, blast cell leukemia, bovine leukemia, chronic myelocytic leukemia, leukemia cutis, embryonal leukemia, eosinophilic leukemia, Gross' leukemia, Rieder cell leukemia, Schilling's leukemia, stem cell leukemia, subleukemic leukemia, undifferentiated cell leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, and promyelocytic leukemi.

The term “carcinoma” refers to a malignant new growth made up of epithelial cells tending to infiltrate the surrounding tissues, and/or resist physiological and non-physiological cell death signals and give rise to metastases. Exemplary carcinomas include, for example, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiennoid carcinoma, carcinoma epitheliale adenoides, exophytic carcinoma, carcinoma ex ulcere, carcinoma fibrosum, gelatiniform carcinoma, gelatinous carcinoma, giant cell carcinoma, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrmcous carcinoma, carcinoma villo sum, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, naspharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, Schneiderian carcinoma, scirrhous carcinoma, and carcinoma scroti,

The term “sarcoma” generally refers to a tumor which is made up of a substance like the embryonic connective tissue and is generally composed of closely packed cells embedded in a fibrillar, heterogeneous, or homogeneous substance. Sarcomas include, chondro sarcoma, fibro sarcoma, lympho sarcoma, melano sarcoma, myxo sarcoma, osteosarcoma, endometrial sarcoma, stromal sarcoma, Ewing's sarcoma, fascial sarcoma, fibroblastic sarcoma, giant cell sarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilns' tumor sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma. Additional exemplary neoplasias include, for example, Hodgkin's Disease, Non-Hodgkin's Lymphoma, multiple myeloma, neuroblastoma, breast cancer, ovarian cancer, lung cancer, rhabdomyo sarcoma, primary thrombocytosis, primary macroglobulinemia, small-cell lung tumors, primary brain tumors, stomach cancer, colon cancer, malignant pancreatic insulanoma, malignant carcinoid, premalignant skin lesions, testicular cancer, lymphomas, thyroid cancer, neuroblastoma, esophageal cancer, genitourinary tract cancer, malignant hypercalcemia, cervical cancer, endometrial cancer, and adrenal cortical cancer.

In certain embodiments, the disorder is a hematological malignancy. In certain instances, the hematological malignancy is via differentiation of hematopoietic cells.

In certain embodiments, the hematologic malignancy is selected from the group consisting of acute myeloid leukemia, chronic myelogenous leukemia (CML), accelerated CML, CML blast phase (CML-BP), acute lymphoblastic leukemia, chronic lymphocytic leukemia (CLL), Hodgkin's disease, non-Hodgkin's lymphoma, follicular lymphoma, mantle cell lymphoma, B-cell lymphoma, T-cell lymphoma, multiple myeloma, Waldenstrom's macroglobulinemia, myelodysplastic syndromes (MDS), refractory anemia (RA), RA with ringed sideroblasts, RA with excess blasts (RAEB), RAEB in transformation, and a myeloproliferative syndrome.

In certain embodiments, the disorder is cancer. An inhibition of NR2F6 according to the present disclosure can be used for immunotherapies for treating cancer.

In certain embodiments, the cancer is a solid tumor selected from adenocarcinoma of the lung, bile duct cancer, bladder cancer; bone cancer, brain tumor, glioma, anaplastic oligodendroglioma, adult glioblastoma multiforme, adult anaplastic astrocytoma; benign prostate hyperplasia bronchoalveolar carcinoma, breast cancer, including metastatic breast cancer; cervical cancer, cholangiocarcinoma, colorectal cancer, esophageal cancer, gastric cancer, head and neck cancer, squamous cell carcinoma of the head and neck, gallbladder cancer hepatocellular cancer, kidney cancer, liver cancer, lung cancer, melanoma; neuroendocrine cancer, metastatic neuroendocrine tumor, non-small cell lung cancer (NSCLC), small cell lung cancer, ovarian cancer, primary peritoneal cancer, pancreatic cancer, prostate cancer, including androgen-dependent and androgen-independent prostate cancer, colorectal carcinoma, renal cancer, metastatic renal cell carcinoma, soft tissue sarcoma, urinary bladder cancer, and uterine cancer.

In certain embodiments, the reaction, disease or disorder comprises an autoimmune disease. An inhibition of NR2F6 according to the present disclosure can be used for treating an augmented autoimmune response. An “augmented immune response” is characterized by a particularly strong response/reaction of the immune system to the presence of an antigen. Under normal, non-pathological conditions, immune responses are regulated in a tightly controlled fashion. Moreover, immune responses are self-limiting and decline in time after exposure to the antigen. In case of an “augmented immune response” however, the immune response may be hypersensitive, i.e. the immune response may cause damage to the organism's own cells/tissue in presence of an antigen. Furthermore, in some cases of an “augmented immune response” for example in autoimmune diseases/disorders or in transplant rejects (and the like), the immune system may fail to distinguish between self and non-self substances. The term “disease related to an augmented immune response”, accordingly, relates to any disease/disorder in which an “augmented immune response” as defined herein above is etiological for, associated with, secondary to or the resultant of said disorder. An augmented immune response may be determined by directly or indirectly measuring parameters which are indicative for the magnitude of the immune response/reaction to an antigen and comparing the outcome of said measurement raised in a to be tested subject with the outcome of the same test in a physiologically normal subject. Parameters indicative for the magnitude of the immune response/reaction may include, but are not limited to the presence/quantity of (specific) antibodies, presence/quantity of (specific) immune cells, the presence/quantity of (specific) cytokines and/or the presence/quantity of (specific) regulatory, activation, and/or adhesion molecules. For a disease to be related to an augmented immune response, accordingly, said augmented immune response may be detectable preceding, during or following said disease. In certain embodiments, the augmented autoimmune response is an autoimmune disease. In a preferred embodiment, the disease related to an augmented immune response is selected from the group consisting of acute or chronic transplant rejection, dermatological disease, T- and B-cell-mediated inflammatory disease, graft-versus-host disease and auto-immune disease. In another preferred embodiment, said dermatological disease is psoriasis, atopic dermatitis or contact allergy. In another preferred embodiment, said T- and B-cell-mediated inflammatory disease is asthma or chronic obstructive pulmonary disease (COPD). In yet another preferred embodiment, said graft-versus-host disease is acute (or fulminant) graft-versus-host disease or chronic graft-versus-host disease. In certain embodiments, said auto-immune disease is multiple sclerosis, inflammatory bowel disease, like ulcerative colitis or Behcet's disease; lupus erythematosus, pemphigus vulgaris, pemphigus foliaceus, myasthenia gravis, polymyositis, mixed collective tissue disease (MCTD) rheumatoid arthritis, diabetes mellitus, celiac disease, atherosclerosis, Goodpasture's syndrome, Grave's disease, autoimmune hepatitis/hepatic autoimmune diseases, autoimmune thrombocytopenic purpura, granulomatosis (e.g. morbus Wegener), or autoimmune haemolytic anaemia. In certain embodiments, the augmented autoimmune response is rheumatoid arthritis, systemic lupus erythematosiss (lupus), inflammatory bowel disease, multiple sclerosis, type-1 diabetes mellitus, Guillian-Barre syndrome, chronic inflammatory demyelinating polyneuropathy, psoriasis/psoriatic arthritis, Grave's disease, Hashimoto's thyroiditis, myasthenia gravis, or vasculitis.

In certain embodiments, the disorder is gastrointestinal disorder. Examples of gastrointestinal disorder include peptic ulcers, regional enteritis, diverticulitis, gastrointestinal bleeding, eosinophilic gastrointestinal disorders (e.g., eosinophilic esophagitis, eosinophilic gastritis, eosinophilic gastroenteritis, eosinophilic colitis), gastritis, diarrhea, gastroesophageal reflux disease (GORD, or its synonym GERD), inflammatory bowel disease (IBD) (e.g., Crohn's disease, ulcerative colitis, collagenous colitis, lymphocytic colitis, ischaemic colitis, diversion colitis, Behcet's syndrome, indeterminate colitis), inflammatory bowel syndrome (IBS)), disorders ameliorated by a gastroprokinetic agent (e.g., ileus, postoperative ileus and ileus during sepsis; gastroesophageal reflux disease (GORD, or its synonym GERD), eosinophilic esophagitis, gastroparesis such as diabetic gastroparesis; food intolerances and food allergies and other functional bowel disorders, such as non-ulcerative dyspepsia (NUD). and non-cardiac chest pain (NCCP, including costo-chondritis)).

The present disclosure provides a method of treating a condition associated with hepatic steatosis. The accumulation of excess triglyceride in the liver is known as hepatic steatosis (or fatty liver). This condition is associated with adverse metabolic consequences, such as insulin resistance and dyslipidemia. Fatty liver is frequently found in subjects having excessive alcohol intake and subjects having obesity, diabetes, or hyperlipidemia. However, in the absence of excessive alcohol intake (>10 g/day), nonalcoholic fatty liver disease (NAFLD) can develop. NAFLD refers to a wide spectrum of liver diseases that can progress from simple fatty liver (steatosis), to nonalcoholic steatohepatitis (NASH), to cirrhosis (irreversible, advanced scarring of the liver). All of the stages of NAFLD have in common the accumulation of fat (fatty infiltration) in the liver cells (hepatocytes).

The NAFLD spectrum begins with and progresses from its simplest stage, called simple fatty liver (steatosis). Simple fatty liver involves the accumulation of fat (triglyceride) in the liver cells with no inflammation (hepatitis) or scarring (fibrosis). The next stage and degree of severity in the NAFLD spectrum is NASH, which involves the accumulation of fat in the liver cells, as well as inflammation of the liver. The inflammatory cells destroy liver cells (hepatocellular necrosis), and NASH ultimately leads to scarring of the liver (fibrosis), followed by irreversible, advanced scarring (cirrhosis). Cirrhosis that is caused by NASH is the last and most severe stage in the NAFLD spectrum.

As used herein, “treating” or “treat” describes the management and care of a patient for the purpose of reversing, inhibiting, or combating a disease, condition, or disorder and includes the administration of a compound of the present disclosure (i.e., a compound of Formula (I-A), (II-A), (I), (II), or (III)), or a pharmaceutically acceptable salt, prodrug, metabolite, polymorph or solvate thereof, to reverse the disease, condition, or disorder, eliminate the disease, condition, or disorder, or inhibit the process of the disease, condition, or disorder.

A compound of the present disclosure (i.e., a compound of Formula (I-A), (II-A), (I), (II), or (III)), or a pharmaceutically acceptable salt, prodrug, metabolite, polymorph, or solvate thereof, can also be used to prevent a disease, condition, or disorder or one or more symptoms of such disease, condition, or disorder. As used herein, “preventing” or “prevent” describes reducing or eliminating the onset of the symptoms or complications of the disease, condition, or disorder.

A compound of the present disclosure (i.e., a compound of Formula (I-A), (II-A), (I), (II), or (III)), or a pharmaceutically acceptable salt, prodrug, metabolite, polymorph, or solvate thereof, can also be used to alleviate one or more symptoms of such disease, condition, or disorder. As used herein, the term “alleviate” is meant to describe a process by which the severity of a sign or symptom of a disorder is decreased. Importantly, a sign or symptom can be alleviated without being eliminated. Preferably treatment is curative or ameliorating.

### Kits

In some embodiments, this disclosure also provides a pharmaceutical package or kit comprising one or more containers filled with at least one compound or composition of this disclosure. Optionally associated with such a container(s) can be a notice in the form prescribed by a governmental agency regulating the manufacture, use or sale of pharmaceuticals or biological products, which notice reflects (a) approval by the agency of manufacture, use or sale for human administration, (b) directions for use, or both. In some embodiments, the kit comprises at least two containers, at least one of which contains at least one compound or composition of this disclosure. In some embodiments, the kit contains at least two containers, and each of the at least two containers contains at least one compound or composition of this disclosure.

In some embodiments, the kit includes additional materials to facilitate delivery of the subject compounds and compositions. For example, the kit may include one or more of a catheter, tubing, infusion bag, syringe, and the like. In some embodiments, the compounds and compositions are packaged in a lyophilized form, and the kit includes at least two containers: a container comprising the lyophilized compounds or compositions and a container comprising a suitable amount of water, buffer, or other liquid suitable for reconstituting the lyophilized material.

The foregoing applies to any of the compounds, compositions, methods, and uses described herein. This disclosure specifically contemplates any combination of the features of such compounds, compositions, methods, and uses (alone or in combination) with the features described for the various kits described in this section.

### ENUMERATED EMBODIMENTS

Embodiment I-1. A compound of Formula (I) or (II):

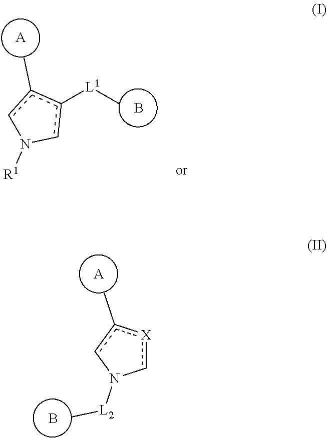

or a pharmaceutically acceptable salt or tautomer thereof, wherein:

each [symbol] independently represents a single bond or a double bond;

X is N, NH, C, CH, or CH₂;

R¹ is H, C₁₋₆alkyl, cycloalkyl, heterocyclyl, —C(O)R^{1a}, —CH₂-aryl, —CH₂-heteroaryl, aryl, or heteroaryl; wherein R^{1a} is C₁₋₆alkyl; and wherein —CH₂-aryl, —CH₂-heteroaryl, aryl, and heteroaryl are optionally substituted with C₁₋₆alkyl or halo;

A is alkyl, cycloalkyl, heterocyclyl, a fused bicyclic aryl, a fused bicyclic heteroaryl, —CH₂-aryl, —CH₂-heteroaryl, aryl, or heteroaryl; wherein the aryl or heteroaryl is optionally substituted with aryl, heteroaryl, —Y^{A}-aryl, or —Y^{A}-heteroaryl; wherein Y^{A} is —O—, —C(O)—, —N(R^{A1})—, —S(O)—, or —S(O)₂—; wherein R^{A1} is H or C₁₋₆alkyl;
wherein the fused bicyclic aryl, the fused bicyclic heteroaryl, —CH₂-aryl, —CH₂— heteroaryl, each aryl, and each heteroaryl are optionally substituted with one or more substituents selected from the group consisting of alkyl, halo, —CN, N(R^{A})₂, —OH, and —O-alkyl; wherein each R^{A} is independently H or C₁₋₆alkyl;

L¹ is —C(O)—NR^{L1}—, —O—C(S)—NR^{L1}—, —O—C(O)—NR^{L1}—, —NR^{L1}—C(O)—, —NR^{L1}—C(O)—O—, —NH—C(O)—NH—, —NR^{L1}—C(S)—NR^{L1}—, —NR^{L1}—S(O)₂—, —S(O)₂—NR^{L1}—, —CH₂—CH₂—, —CH₂—NR^{L1}—, —NR^{L1}—CH₂—, —CH₂—O—, —O—CH₂—, —O—, —NH—, —C(O)-azetidinyl, —CH₂—NR^{L1}—C(O)—, or —C(O)—NR^{L1}—CH₂—; wherein each R^{L1} is independently H or C₁₋₆alkyl; and

L² is —C(O)—NR^{L2}—, —S(O)₂—NR^{L2}—, —CH₂—CH₂—, —C(S)—NR^{L2}—, —C(O)—, or —S(O)₂—; wherein each R^{L2} is independently H or C₁₋₆alkyl; and

B is a fused bicyclic aryl, a fused bicyclic heteroaryl, —CH₂-aryl, —CH₂-heteroaryl, aryl, heteroaryl, cycloalkyl, or —CH₂-heterocyclyl, wherein the aryl or heteroaryl is optionally substituted with aryl, heteroaryl, —Y^{B}-aryl, or —Y^{B}-heteroaryl; wherein Y^{B} is —O—, —C(O)—, —N(R^{B1})—, —S(O)—, or —S(O)₂—; wherein R^{B1} is H or C₁₋₆alkyl;
wherein the fused bicyclic aryl, the fused bicyclic heteroaryl, —CH₂-aryl, —CH₂— heteroaryl, each aryl, each heteroaryl, cycloalkyl, and —CH₂-heterocyclyl are optionally substituted with one or more substituents selected from the group consisting of alkyl, halo, —CN, —N(R^{B2})₂, —OH, and —O-alkyl; wherein each R^{B2} is independently H or C₁₋₆alkyl;

wherein when the compound is Formula (I); A is optionally substituted phenyl or thiophenyl, and L¹ is —C(O)—NH—; then B is not

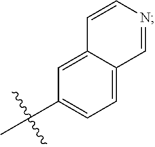 [SMILES: *c1ccc2cnccc2c1]

wherein when the compound is Formula (I); A is a substituted phenyl and B is a substituted phenyl, then L¹ is not —C(O)—NH—, —NH—C(O)—, —NCH₃—C(O)—, or —NH—C(O)—NH—;

wherein when the compound is Formula (I); B is optionally substituted —CH₂-aryl and A is optionally substituted aryl; then L¹ is not —C(O)—NH—;

wherein when the compound is Formula (II); A is optionally substituted phenyl and B is optionally substituted phenyl, then L¹ is not —C(O)—NCH₃—.

Embodiment I-2. A compound of Formula (III):

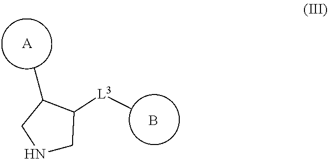

or a pharmaceutically acceptable salt or tautomer thereof, wherein:

A is aryl or 5- to 6-membered heteroaryl, wherein the aryl and heteroaryl are optionally substituted with one or more substituents selected from the group consisting of alkyl, halo, —OH, and —O-alkyl;

L³ is —C(O)—NR^{L3}—, —O—C(S)—NR^{L3}—, —O—C(O)—NR^{L3}—, —NR^{L3}—C(O)—, —NR^{L3}—C(S)—NR^{L3}—, —NR^{L3}—S(O)₂—, —S(O)₂—NR^{L3}—, —CH₂—CH₂—, —CH₂—NR^{L3}—, —NR^{L3}—CH₂—, —CH₂—O—, —O—CH₂—, or —O—; wherein each R^{L3} is independently hydrogen or C₁₋₆alkyl; and

B is a fused bicyclic aryl, a fused bicyclic heteroaryl, —CH₂-aryl, —CH₂-heteroaryl, aryl, or heteroaryl, wherein the aryl or heteroaryl is optionally substituted with aryl or heteroaryl;

wherein the fused bicyclic aryl, the fused bicyclic heteroaryl, —CH₂-aryl, —CH₂— heteroaryl, each aryl, and each heteroaryl are optionally substituted with one or more substituents selected from the group consisting of alkyl, halo, —OH, and —O-alkyl;

wherein when A is optionally substituted phenyl or thiophenyl, and L³ is —C(O)—NH—; then B is not

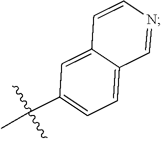 [SMILES: *c1ccc2cnccc2c1]

wherein when A is a substituted phenyl and B is a substituted phenyl, then L³ is not —C(O)—NH—, —NH—C(O)—, —NCH₃—C(O)—, or —NH—C(O)—NH—;

wherein when the compound is Formula (I); B is optionally substituted —CH₂-aryl and A is optionally substituted aryl; then L³ is not —C(O)—NH—.

Embodiment I-3. A compound of Formula (IV):

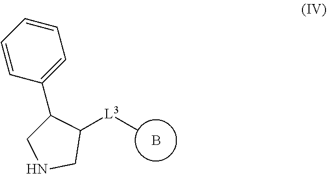

or a pharmaceutically acceptable salt or tautomer thereof, wherein:

L³ is —C(O)—NR^{L3}—, —O—C(S)—NR^{L3}—, —O—C(O)—NR^{L3}—, —NR^{L3}—C(O)—, —NR^{L3}—C(S)—NR^{L3}—, —NR^{L3}—S(O)₂—, —S(O)₂—NR^{L3}—, —CH₂—CH₂—, —CH₂—NR^{L3}—, —NR^{L3}—CH₂—, —CH₂—O—, —O—CH₂—, or —O—; wherein each R^{L3} is independently hydrogen or C₁₋₆alkyl; and

B is a fused bicyclic aryl, a fused bicyclic heteroaryl, —CH₂-aryl, —CH₂-heteroaryl, aryl, or heteroaryl, wherein the aryl or heteroaryl is optionally substituted with aryl or heteroaryl;

wherein the fused bicyclic aryl, the fused bicyclic heteroaryl, —CH₂-aryl, —CH₂— heteroaryl, each aryl, and each heteroaryl are optionally substituted with one or more substituents selected from the group consisting of alkyl, halo, —OH, and —O-alkyl;

wherein when L³ is —C(O)—NH—; then B is not

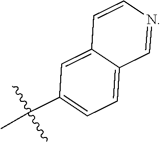 [SMILES: *c1ccc2cnccc2c1]

Embodiment I-4. A compound of Formula (V):

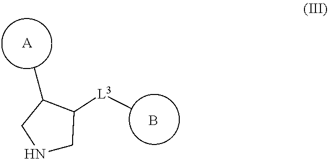

or a pharmaceutically acceptable salt or tautomer thereof, wherein:

A is aryl or 5- to 6-membered heteroaryl, wherein the aryl and heteroaryl are optionally substituted with one or more substituents selected from the group consisting of alkyl, halo, —OH, and —O-alkyl;

L³ is —C(O)—NR^{L3}—, —O—C(S)—NR^{L3}—, —O—C(O)—NR^{L3}—, —NR^{L3}—C(O)—, —NR^{L3}—C(S)—NR^{L3}—, —NR^{L3}—S(O)₂—, —S(O)₂—NR^{L3}—, —CH₂—CH₂—, —CH₂—NR^{L3}—, —NR^{L3}—CH₂—, —CH₂—O—, —O—CH₂—, or —O—; wherein each R^{L3} is independently hydrogen or C₁₋₆alkyl; and

B1 is a fused bicyclic aryl or a fused bicyclic heteroaryl; wherein the fused bicyclic aryl and the fused bicyclic heteroaryl are optionally substituted with one or more substituents selected from the group consisting of alkyl, halo, —OH, and —O-alkyl;

wherein when A is optionally substituted phenyl or thiophenyl, and L³ is —C(O)—NH—; then B is not

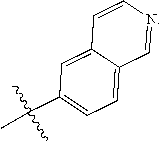 [SMILES: *c1ccc2cnccc2c1]

Embodiment I-5. The compound of embodiment I-4, or a pharmaceutically acceptable salt or tautomer thereof, wherein B1 is a fused bicyclic aryl.

Embodiment I-6. The compound of embodiment I-4, or a pharmaceutically acceptable salt or tautomer thereof, wherein B1 is a fused bicyclic heteroaryl.

Embodiment I-7. The compound of embodiment I-4, or a pharmaceutically acceptable salt or tautomer thereof, wherein B1 is selected from the group consisting of

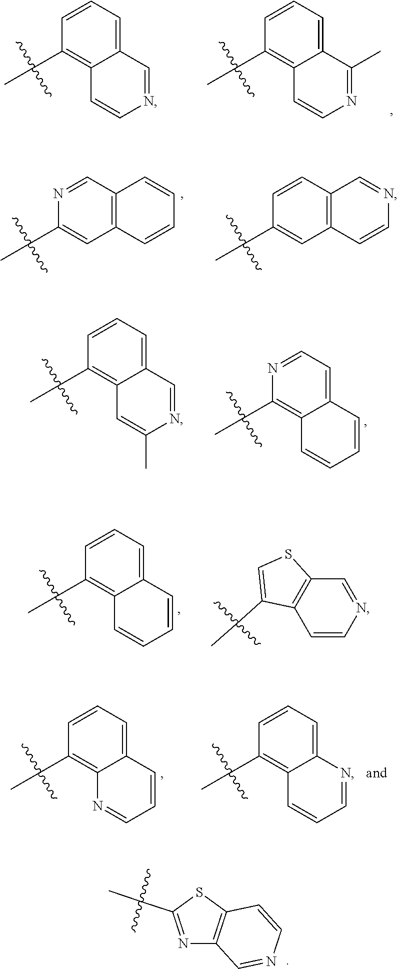

Embodiment I-8. A compound of Formula (VI):

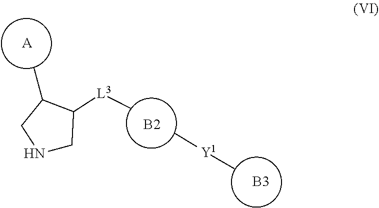

or a pharmaceutically acceptable salt or a tautomer thereof, wherein:

A is aryl or 5- to 6-membered heteroaryl, wherein the aryl and heteroaryl are optionally substituted with one or more substituents selected from the group consisting of alkyl, halo, —OH, and —O-alkyl;

L³ is —C(O)—NR^{L3}—, —O—C(S)—NR^{L3}—, —O—C(O)—NR^{L3}—, —NR^{L3}—C(O)—, —NR^{L3}—C(S)—NR^{L3}—, —NR^{L3}—S(O)₂—, —S(O)₂—NR^{L3}—, —CH₂—CH₂—, —CH₂—NR^{L3}—, —NR^{L3}—CH₂—, —CH₂—O—, —O—CH₂—, or —O—; wherein each R^{L3} is independently hydrogen or C₁₋₆alkyl; and

B2 is monocyclic aryl or monocyclic heteroaryl; wherein the aryl and heteroaryl are optionally substituted with one or more substituents selected from the group consisting of alkyl, halo, —OH, and —O-alkyl;

Y¹ is absent, —O—, —C(O)—, —N(R^{Y})—, —S(O)—, or —S(O)₂—; wherein R^{Y} is H or C₁₋₆alkyl; and

B3 is monocyclic aryl or monocyclic heteroaryl; wherein the aryl and heteroaryl are optionally substituted with one or more substituents selected from the group consisting of alkyl, halo, —OH, and —O-alkyl.

Embodiment I-9. The compound of embodiment I-8, or a pharmaceutically acceptable salt or tautomer thereof, wherein B2 is monocyclic aryl.

Embodiment I-10. The compound of embodiment I-8, or a pharmaceutically acceptable salt or tautomer thereof, wherein B2 is monocyclic heteroaryl.

Embodiment I-11. The compound of embodiment I-8, or a pharmaceutically acceptable salt or tautomer thereof, wherein B3 is monocyclic aryl.

Embodiment I-12. The compound of embodiment I-8, or a pharmaceutically acceptable salt or tautomer thereof, wherein B3 is monocyclic heteroaryl.

Embodiment I-13. The compound of embodiment I-8, or a pharmaceutically acceptable salt or tautomer thereof, wherein

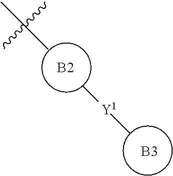

is selected from the group consisting of

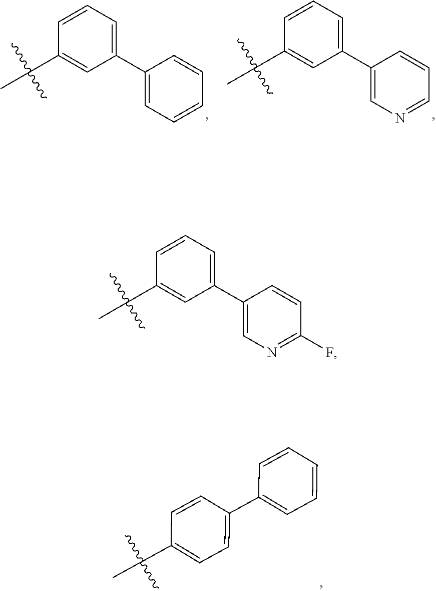 [SMILES: *c1cccc(-c2ccccc2)c1 ; *c1cccc(-c2cccnc2)c1 ; *c1cccc(-c2ccc(F)nc2)c1 ; *c1ccc(-c2ccccc2)cc1]

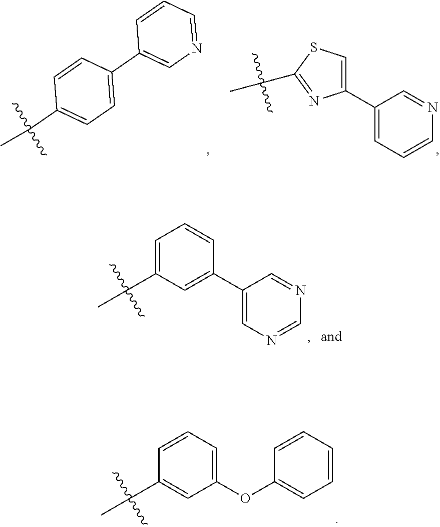 [SMILES: *c1ccc(-c2cccnc2)cc1 ; *c1nc(-c2cccnc2)cs1 ; *c1cccc(-c2cncnc2)c1 ; *c1cccc(Oc2ccccc2)c1]

Embodiment I-14. A compound of Formula (VII):

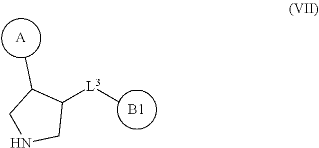

or a pharmaceutically acceptable salt or tautomer thereof, wherein:

A is aryl or 5- to 6-membered heteroaryl, wherein the aryl and heteroaryl are optionally substituted with one or more substituents selected from the group consisting of alkyl, halo, —OH, and —O-alkyl;

L³ is —C(O)—NR^{L3}—, —O—C(S)—NR^{L3}—, —O—C(O)—NR^{L3}—, —NR^{L3}—C(O)—, —NR^{L3}—C(S)—NR^{L3}—, —NR^{L3}—S(O)₂—, —S(O)₂—NR^{L3}—, —CH₂—CH₂—, —CH₂—NR^{L3}—, —NR^{L3}—CH₂—, —CH₂—O—, —O—CH₂—, or —O—; wherein each R^{L3} is independently hydrogen or C₁₋₆alkyl; and

B1 is a fused bicyclic aryl or a fused bicyclic heteroaryl; wherein the fused bicyclic aryl and the fused bicyclic heteroaryl are optionally substituted with one or more substituents selected from the group consisting of alkyl, halo, —OH, and —O-alkyl;

wherein when A is optionally substituted phenyl or thiophenyl, and L³ is —C(O)—NH—; then B is not

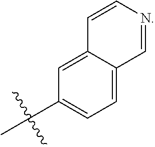 [SMILES: *c1ccc2cnccc2c1]

Embodiment I-15. The compound of embodiment I-14, or a pharmaceutically acceptable salt or tautomer thereof, wherein B4 is —CH₂-aryl.

Embodiment I-16. The compound of embodiment I-14, or a pharmaceutically acceptable salt or tautomer thereof, wherein B4 is —CH₂-heteroaryl.

Embodiment I-17. The compound of embodiment I-14, or a pharmaceutically acceptable salt or tautomer thereof, wherein B4 is selected from the group consisting of

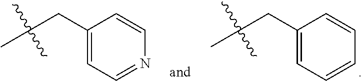 [SMILES: *Cc1ccncc1 ; *Cc1ccccc1]

Embodiment I-18. The compound of embodiment I-1, or a pharmaceutically acceptable salt or tautomer thereof, wherein

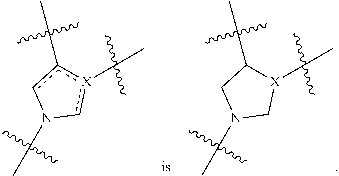

Embodiment I-19. The compound of embodiment I-1, or a pharmaceutically acceptable salt or tautomer thereof, wherein

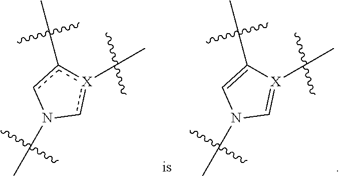

Embodiment I-20. The compound of embodiment I-1, or a pharmaceutically acceptable salt or tautomer thereof, wherein

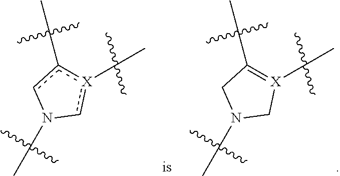

Embodiment I-21. The compound of any one of embodiments I-1 and I-18 to I-20, or a pharmaceutically acceptable salt or tautomer thereof, wherein X is N or NH.

Embodiment I-22. The compound of any one of embodiments I-1 and I-18 to I-20, or a pharmaceutically acceptable salt or tautomer thereof, wherein X is C, CH, or CEE.

Embodiment I-23. The compound of any one of embodiments I-1 and I-18 to I-22, or a pharmaceutically acceptable salt or tautomer thereof, wherein R¹ is H.

Embodiment I-24. The compound of any one of embodiments I-1 and I-18 to I-22, or a pharmaceutically acceptable salt or tautomer thereof, wherein R¹ is C₁₋₆alkyl.

Embodiment I-25. The compound of any one of embodiments I-1 and I-18 to I-22, or a pharmaceutically acceptable salt or tautomer thereof, wherein R¹ is cycloalkyl.

Embodiment I-26. The compound of any one of embodiments I-1 and I-18 to I-22, or a pharmaceutically acceptable salt or tautomer thereof, wherein R¹ is heterocyclyl.

Embodiment I-27. The compound of any one of embodiments I-1 and I-18 to I-22, or a pharmaceutically acceptable salt or tautomer thereof, wherein R¹ is —C(O)R^{1a}.

Embodiment I-28. The compound of any one of embodiments I-1 and I-18 to I-22, or a pharmaceutically acceptable salt or tautomer thereof, wherein R¹ is —CH₂-aryl.

Embodiment I-29. The compound of any one of embodiments I-1 to I-28, or a pharmaceutically acceptable salt thereof, wherein A is aryl.

Embodiment I-30. The compound of embodiment I-29, or a pharmaceutically acceptable salt or tautomer thereof, wherein the aryl is substituted with one or more substituents selected from the group consisting of alkyl, halo, —OH, and —O-alkyl.

Embodiment I-31. The compound of any one of embodiments I-1 to I-28, or a pharmaceutically acceptable salt or tautomer thereof, wherein A is 5- to 6-membered heteroaryl.

Embodiment I-32. The compound of embodiment I-31, or a pharmaceutically acceptable salt or tautomer thereof, wherein the heteroaryl is substituted with one or more substituents selected from the group consisting of alkyl, halo, —OH, and —O-alkyl.

Embodiment I-33. The compound of any one of embodiments I-1 to I-28, or a pharmaceutically acceptable salt or tautomer thereof, wherein A is alkyl.

Embodiment I-34. The compound of any one of embodiments I-1 to I-28, or a pharmaceutically acceptable salt or tautomer thereof, wherein A is cycloalkyl.

Embodiment I-35. The compound of any one of embodiments I-1 to I-28, or a pharmaceutically acceptable salt or tautomer thereof, wherein A is heterocyclyl.

Embodiment I-36. The compound of any one of embodiments I-1 to I-28, or a pharmaceutically acceptable salt or tautomer thereof, wherein A is a fused bicyclic aryl or a fused bicyclic heteroaryl.

Embodiment I-37. The compound of any one of embodiments I-1 to I-28, or a pharmaceutically acceptable salt or tautomer thereof, wherein A is —CH₂-aryl or —CH₂— heteroaryl.

Embodiment I-38. The compound of any one of embodiments I-1 and I-17 to I-37, or a pharmaceutically acceptable salt or tautomer thereof, wherein L¹ is —C(O)—NR^{L1}—.

Embodiment I-39. The compound of any one of embodiments I-1 and I-17 to I-37, or a pharmaceutically acceptable salt or tautomer thereof, wherein L¹ is —O—C(S)—NR^{L1}—.

Embodiment I-40. The compound of any one of embodiments I-1 and I-17 to I-37, or a pharmaceutically acceptable salt or tautomer thereof, wherein L¹ is —O—C(O)—NR^{L1}—.

Embodiment I-41. The compound of any one of embodiments I-1 and I-17 to I-37, or a pharmaceutically acceptable salt or tautomer thereof, wherein L¹ is —NR^{L1}—C(S)—NR^{L1}—.

Embodiment I-42. The compound of any one of embodiments I-1 and I-17 to I-37, or a pharmaceutically acceptable salt or tautomer thereof, wherein L¹ is —O—.

Embodiment I-43. The compound of any one of embodiments I-1 and I-17 to I-37, or a pharmaceutically acceptable salt or tautomer thereof, wherein L¹ is —NR^{L1}—C(O)—, —NR^{L1}—C(O)—O—, —NH—C(O)—NH—, —NR^{L1}—S(O)₂—, or —S(O)₂—NR^{L1}—.

Embodiment I-44. The compound of any one of embodiments I-1 and I-17 to I-37, or a pharmaceutically acceptable salt or tautomer thereof, wherein L¹ is —CH₂—CH₂—, —CH₂—NR^{L1}—, —NR^{L1}—CH₂—, —CH₂—O—, —O—CH₂—, —NH—, or —C(O)-azetidinyl.

Embodiment I-45. The compound of any one of embodiments I-1 and I-17 to I-37, or a pharmaceutically acceptable salt or tautomer thereof, wherein L² is —C(O)—NR^{L2}—.

Embodiment I-46. The compound of any one of embodiments I-1 and I-17 to I-37, or a pharmaceutically acceptable salt or tautomer thereof, wherein L² is —S(O)₂—NR^{L2}— or —CH₂—CH₂—.

Embodiment I-47. The compound of any one of embodiments I-2 to I-17 and I-29 to I-37, or a pharmaceutically acceptable salt or tautomer thereof, wherein L³ is —C(O)—NR^{L3}—.

Embodiment I-48. The compound of any one of embodiments I-2 to I-17 and I-29 to I-37, or a pharmaceutically acceptable salt or tautomer thereof, wherein L³ is —O—C(S)—NR^{L3}—.

Embodiment I-49. The compound of any one of embodiments I-2 to I-17 and I-29 to I-37, or a pharmaceutically acceptable salt or tautomer thereof, wherein L³ is —O—C(O)—NR^{L3}—.

Embodiment I-50. The compound of any one of embodiments I-2 to I-17 and I-29 to I-37, or a pharmaceutically acceptable salt or tautomer thereof, wherein L³ is —NR^{L3}—C(S)—NR^{L3}—.

Embodiment I-51. The compound of any one of embodiments I-2 to I-17 and I-29 to I-37, or a pharmaceutically acceptable salt or tautomer thereof, wherein L³ is —NR^{L3}—C(O)—, —NR^{L3}—S(O)₂—, —S(O)₂—NR^{L3}—, —CH₂—CH₂—, —CH₂—NR^{L3}—, or —NR^{L3}—CH₂—.

Embodiment I-52. The compound of any one of embodiments I-2 to I-17 and I-29 to I-37, or a pharmaceutically acceptable salt or tautomer thereof, wherein L³ is —CH₂—O—, —O—CH₂—, or —O—.

Embodiment I-53. The compound of any one of embodiments I-1 to I-3 and I-18 to I-52, or a pharmaceutically acceptable salt or tautomer thereof, wherein B is a fused bicyclic aryl.

Embodiment I-54. The compound of any one of embodiments I-1 to I-3 and I-18 to I-52, or a pharmaceutically acceptable salt or tautomer thereof, wherein B is a fused bicyclic heteroaryl.

Embodiment I-55. The compound of any one of embodiments I-1 to I-3 and I-18 to I-52, or a pharmaceutically acceptable salt or tautomer thereof, wherein B is selected from the group consisting of

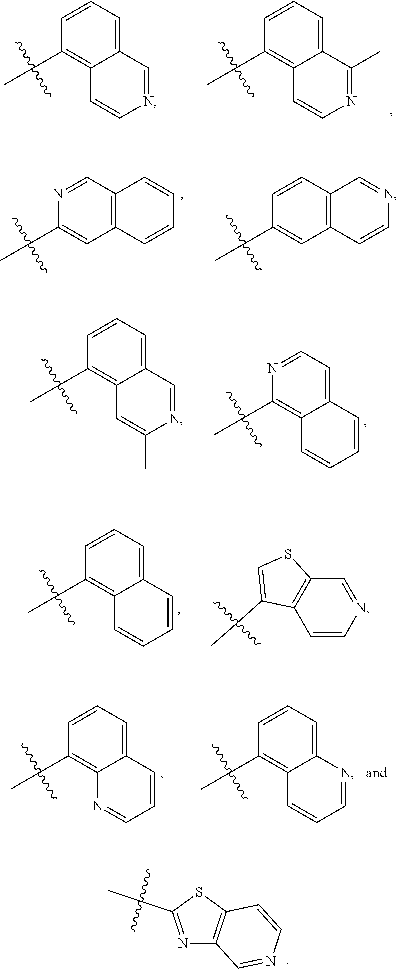

Embodiment I-56. The compound of any one of embodiments I-1 to I-3 and I-18 to I-52, or a pharmaceutically acceptable salt or tautomer thereof, wherein B is —CH₂-aryl.

Embodiment I-57. The compound of any one of embodiments I-1 to I-3 and I-18 to I-52, or a pharmaceutically acceptable salt or tautomer thereof, wherein B is —CH₂— heteroaryl.

Embodiment I-58. The compound of any one of embodiments I-1 to I-3 and I-18 to I-52, or a pharmaceutically acceptable salt or tautomer thereof, wherein B is selected from the group consisting of

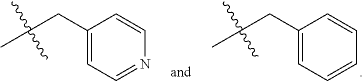 [SMILES: *Cc1ccncc1 ; *Cc1ccccc1]

Embodiment I-59. The compound of any one of embodiments I-1 to I-3 and I-18 to I-51, or a pharmaceutically acceptable salt or tautomer thereof, wherein B is aryl.

Embodiment I-60. The compound of any one of embodiments I-1 to I-3 and I-18 to I-51, or a pharmaceutically acceptable salt or tautomer thereof, wherein B is aryl substituted with aryl or heteroaryl.

Embodiment I-61. The compound of any one of embodiments I-1 to I-3 and I-18 to I-51, or a pharmaceutically acceptable salt or tautomer thereof, wherein B is heteroaryl.

Embodiment I-62. The compound of any one of embodiments I-1 to I-3 and I-18 to I-51, or a pharmaceutically acceptable salt or tautomer thereof, wherein B is heteroaryl substituted with aryl or heteroaryl.

Embodiment I-63. The compound of any one of embodiments I-1 to I-3 and I-18 to I-51, or a pharmaceutically acceptable salt or tautomer thereof, wherein B is selected from the group consisting of

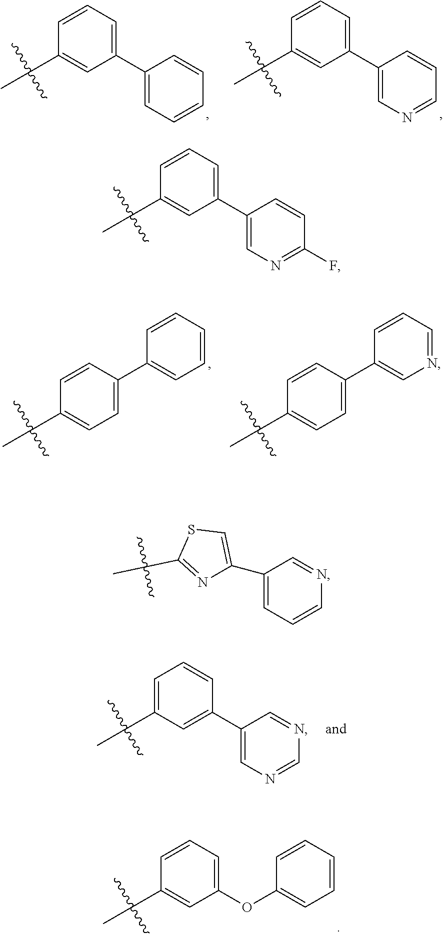

Embodiment I-64. The compound of any one of embodiments I-1 to I-3 and I-18 to I-51, or a pharmaceutically acceptable salt or tautomer thereof, wherein B is cycloalkyl.

Embodiment I-65. The compound of any one of embodiments I-1 to I-3 and I-18 to I-51, or a pharmaceutically acceptable salt or tautomer thereof, wherein B is —CH₂— heterocyclyl.

Embodiment I-66. A compound, or a pharmaceutically acceptable salt or tautomer thereof, selected from the group consisting of

| Compound | |
|---|---|
| No. | Structure |
| I-1 | 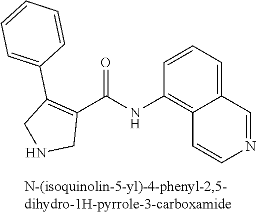 [SMILES: O=C(Nc1cccc2cnccc12)C1=C(c2ccccc2)CNC1] |
| I-2 | 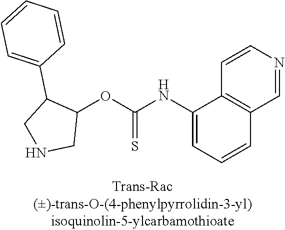 [SMILES: S=C(Nc1cccc2cnccc12)OC1CNCC1c1ccccc1] |
| I-3 | 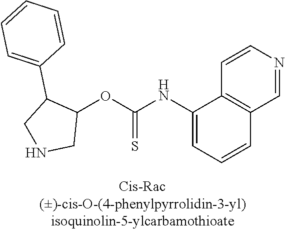 [SMILES: S=C(Nc1cccc2cnccc12)OC1CNCC1c1ccccc1] |
| I-4 | 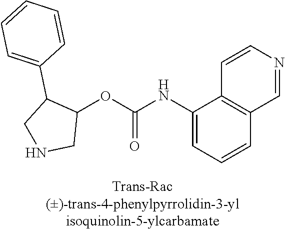 [SMILES: O=C(Nc1cccc2cnccc12)OC1CNCC1c1ccccc1] |
| I-5 | 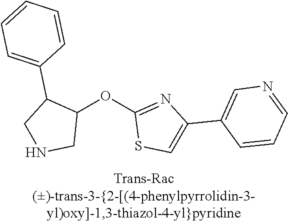 [SMILES: c1ccc(C2CNCC2Oc2nc(-c3cccnc3)cs2)cc1] |
| I-6 | 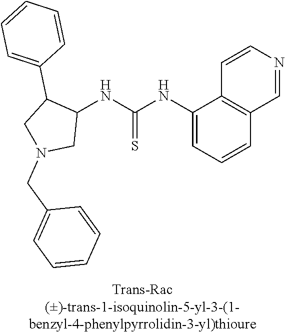 [SMILES: S=C(Nc1cccc2cnccc12)NC1CN(Cc2ccccc2)CC1c1ccccc1] |
| I-7 | 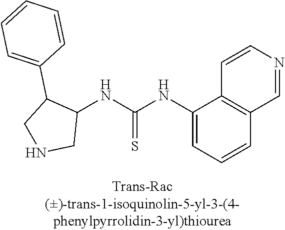 [SMILES: S=C(Nc1cccc2cnccc12)NC1CNCC1c1ccccc1] |
| I-8 | 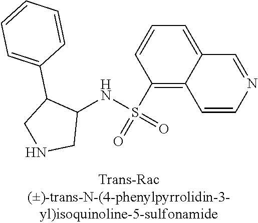 [SMILES: O=S(=O)(NC1CNCC1c1ccccc1)c1cccc2cnccc12] |
| I-9 | 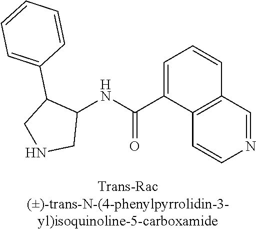 [SMILES: O=C(NC1CNCC1c1ccccc1)c1cccc2cnccc12] |
| I-10 | 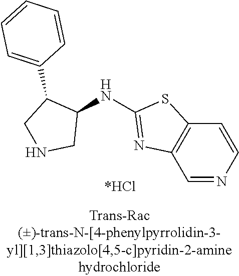 [SMILES: c1ccc([C@H]2CNC[C@@H]2Nc2nc3cnccc3s2)cc1] |
| I-11 | 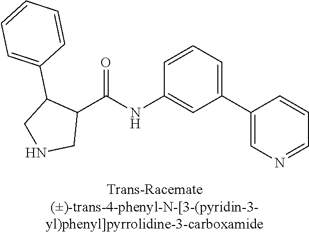 [SMILES: O=C(Nc1cccc(-c2cccnc2)c1)C1CNCC1c1ccccc1] |
| I-12 | 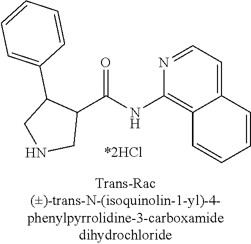 [SMILES: O=C(Nc1nccc2ccccc12)C1CNCC1c1ccccc1] |
| I-13 | 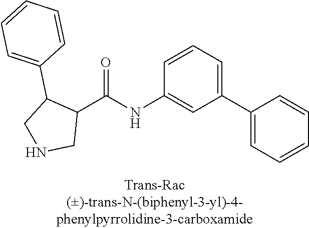 [SMILES: O=C(Nc1cccc(-c2ccccc2)c1)C1CNCC1c1ccccc1] |
| I-14 | 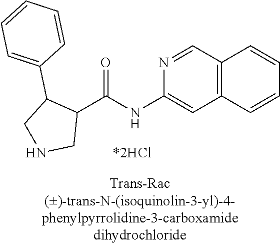 [SMILES: O=C(Nc1cc2ccccc2cn1)C1CNCC1c1ccccc1] |
| I-15 | 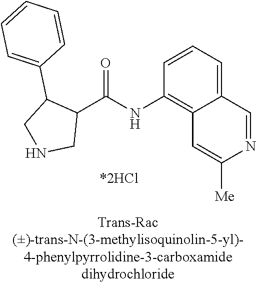 [SMILES: Cc1cc2c(NC(=O)C3CNCC3c3ccccc3)cccc2cn1] |
| I-16 | 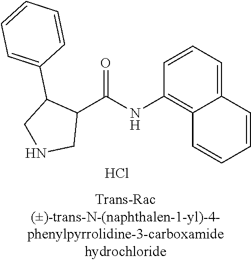 [SMILES: O=C(Nc1cccc2ccccc12)C1CNCC1c1ccccc1 ; Cl] |
| I-17 | 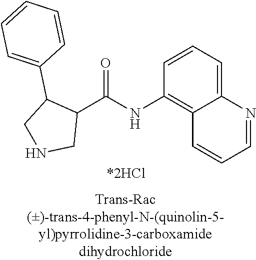 |
| I-18 | 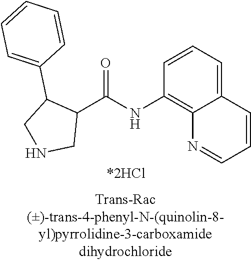 |
| I-19 | 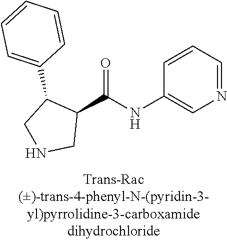 [SMILES: O=C(Nc1cccnc1)[C@H]1CNC[C@@H]1c1ccccc1] |
| I-20 | 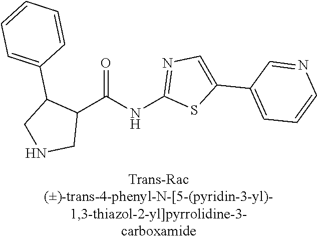 [SMILES: O=C(Nc1ncc(-c2cccnc2)s1)C1CNCC1c1ccccc1] |
| I-21 | 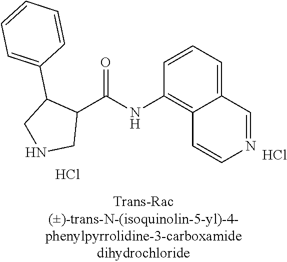 [SMILES: O=C(Nc1cccc2cnccc12)C1CNCC1c1ccccc1 ; Cl ; Cl] |
| I-22 | 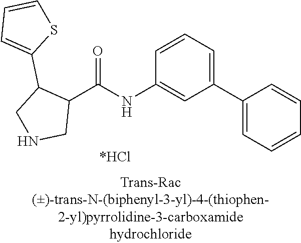 |
| I-23 | 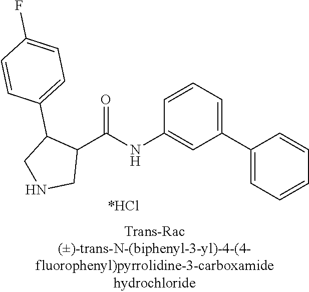 |
| I-24 | 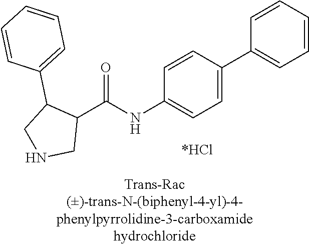 |
| I-25 | 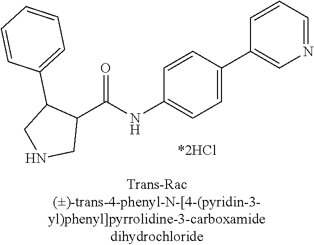 |
| I-26 | 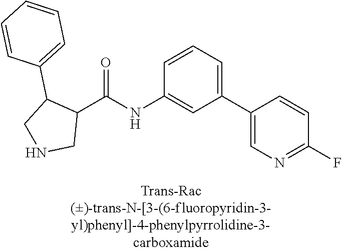 [SMILES: O=C(Nc1cccc(-c2ccc(F)nc2)c1)C1CNCC1c1ccccc1] |
| I-27 | 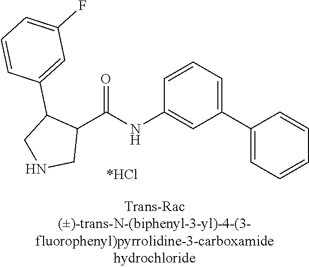 [SMILES: O=C(Nc1cccc(-c2ccccc2)c1)C1CNCC1c1cccc(F)c1] |
| I-28 | 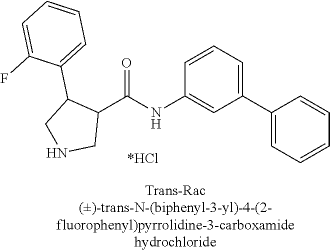 [SMILES: O=C(Nc1cccc(-c2ccccc2)c1)C1CNCC1c1ccccc1F] |
| I-29 | 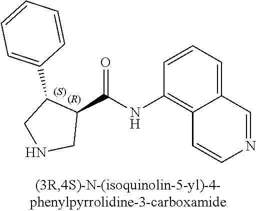 |
| I-30 | 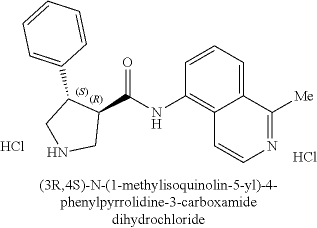 |
| I-31 | 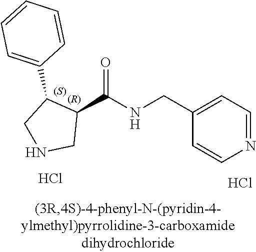 |
| I-32 | 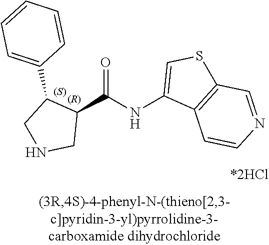 |
| I-33 | 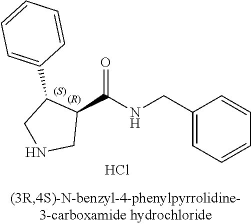 |
| I-34 | 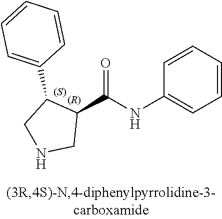 |
| I-35 | 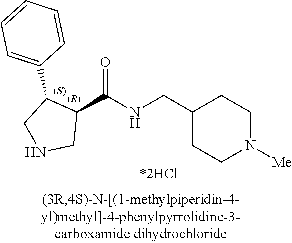 |
| I-36 | 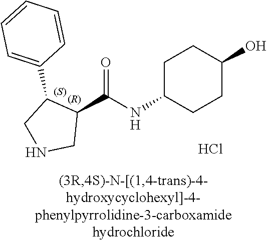 |
| I-37 | 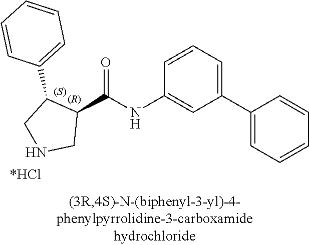 |
| I-38 | 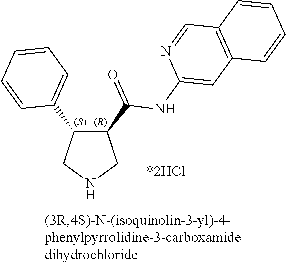 |
| I-39 | 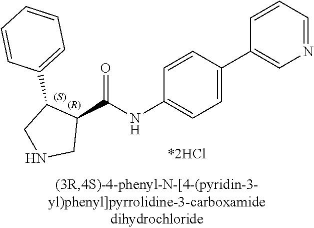 |
| I-40 | 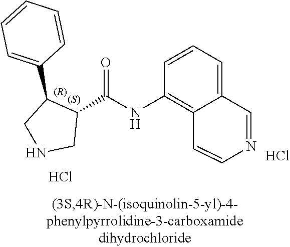 |
| I-41 | 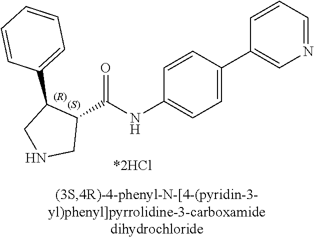 |
| I-42 | 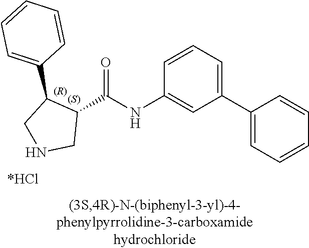 |
| I-43 | 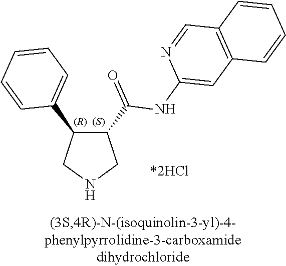 |
| I-44 | 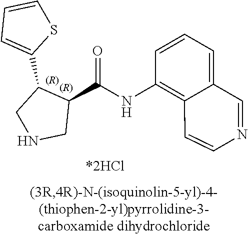 |
| I-45 | 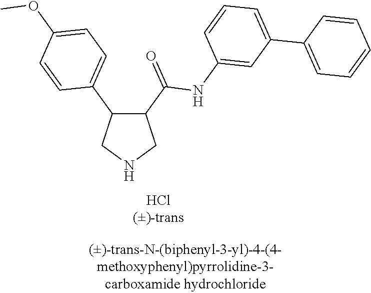 [SMILES: COc1ccc(C2CNCC2C(=O)Nc2cccc(-c3ccccc3)c2)cc1] |
| I-46 | 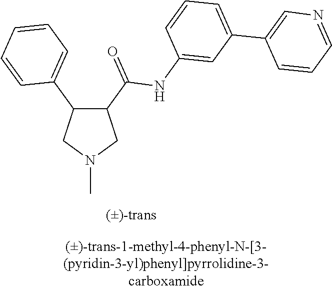 [SMILES: CN1CC(C(=O)Nc2cccc(-c3cccnc3)c2)C(c2ccccc2)C1] |
| I-47 | 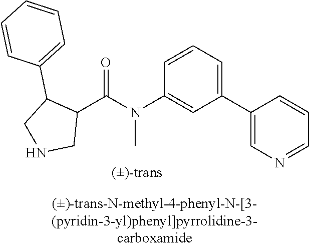 [SMILES: CN(C(=O)C1CNCC1c1ccccc1)c1cccc(-c2cccnc2)c1] |
| I-48 | 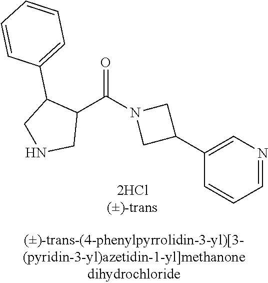 [SMILES: O=C(C1CNCC1c1ccccc1)N1CC(c2cccnc2)C1] |
| I-49 | 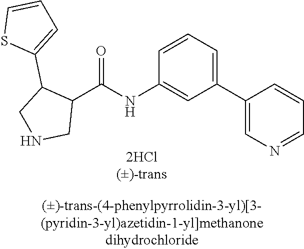 [SMILES: O=C(Nc1cccc(-c2cccnc2)c1)C1CNCC1c1cccs1] |
| I-50 | 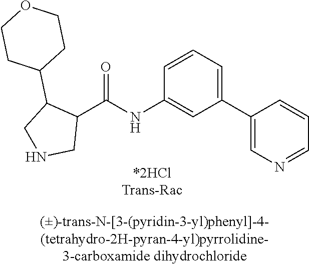 [SMILES: O=C(Nc1cccc(-c2cccnc2)c1)C1CNCC1C1CCOCC1] |
| I-51 | 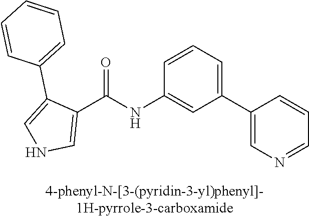 [SMILES: O=C(Nc1cccc(-c2cccnc2)c1)c1c[nH]cc1-c1ccccc1] |
| I-52 | 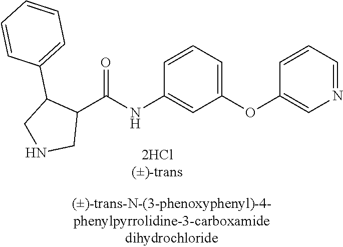 [SMILES: O=C(Nc1cccc(Oc2cccnc2)c1)C1CNCC1c1ccccc1] |
| I-53 | 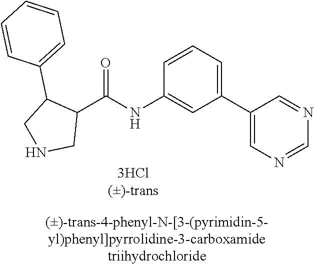 [SMILES: O=C(Nc1cccc(-c2cncnc2)c1)C1CNCC1c1ccccc1] |
| I-54 | 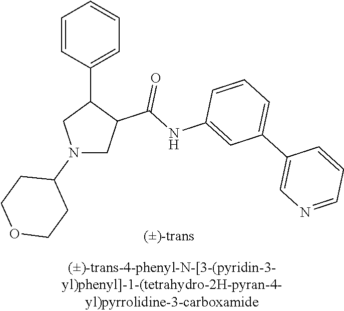 [SMILES: O=C(Nc1cccc(-c2cccnc2)c1)C1CN(C2CCOCC2)CC1c1ccccc1] |
| I-55 | 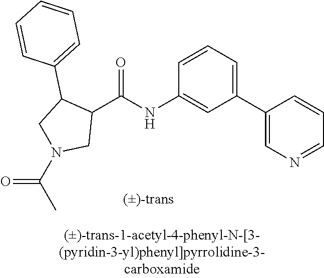 [SMILES: CC(=O)N1CC(C(=O)Nc2cccc(-c3cccnc3)c2)C(c2ccccc2)C1] |
| I-56 | 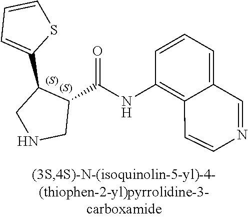 |
| I-57 | 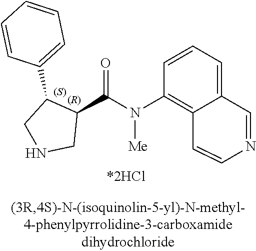 |
| I-58 | 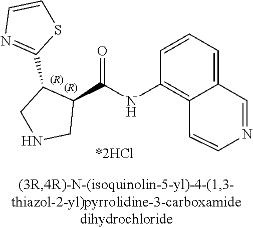 |
| I-59 | 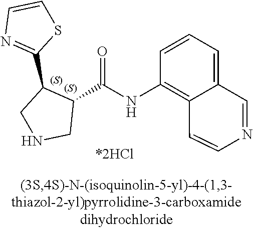 |

Embodiment I-67. A pharmaceutical composition, comprising the compound of any one of embodiments I-1 to I-66, or a pharmaceutically acceptable salt or tautomer thereof, and a pharmaceutically acceptable excipient.

Embodiment I-68. A method of modulating activity of NR2F6 by exposure of NR2F6 to an effective amount of a compound of any one of embodiments I-1 to I-66, or a pharmaceutically acceptable salt or tautomer thereof, or the pharmaceutical composition of embodiment I-67.

Embodiment I-69. The method of embodiment I-68, wherein said modulation comprises of augmentation of NR2F6 activity.

Embodiment I-70. The method of embodiment I-68, wherein said modulation comprise of inhibition of NR2F6 activity.

Embodiment I-71. A method of treating or reducing the effect of a disease or disorder associated with NR2F6 modulation, the method comprising administration of an effective amount of a compound of any one of embodiments I-1 to I-66, or a pharmaceutically acceptable salt or tautomer thereof, or the pharmaceutical composition of embodiment I-67.

Embodiment I-72. The method of embodiment I-71, wherein the disease or disorder comprises an augmented autoimmune response.

Embodiment I-73. The method according to embodiment I-72, wherein the augmented autoimmune response is selected from the group consisting of rheumatoid arthritis, systemic lupus erythematosiss (lupus), inflammatory bowel disease, multiple sclerosis, type-1 diabetes mellitus, Guillian-Barre syndrome, chronic inflammatory demyelinating polyneuropathy, psoriasis/psoriatic arthritis, Grave's disease, Hashimoto's thyroiditis, myasthenia gravis, and vasculitis.

Embodiment I-74. The method of embodiment I-71, wherein the disorder is cancer.

Embodiment I-75. The method according to embodiment I-74, wherein the cancer is a solid tumor selected from the group consisting of adenocarcinoma of the lung, bile duct cancer, bladder cancer; bone cancer, brain tumor, glioma, anaplastic oligodendroglioma, adult glioblastoma multiforme, adult anaplastic astrocytoma; benign prostate hyperplasia bronchoalveolar carcinoma, breast cancer, including metastatic breast cancer; cervical cancer, cholangiocarcinoma, colorectal cancer, esophageal cancer, gastric cancer, head and neck cancer, squamous cell carcinoma of the head and neck, gallbladder cancer hepatocellular cancer, kidney cancer, liver cancer, lung cancer, melanoma; neuroendocrine cancer, metastatic neuroendocrine tumor, non-small cell lung cancer (NSCLC), small cell lung cancer, ovarian cancer, primary peritoneal cancer, pancreatic cancer, prostate cancer, including androgen-dependent and androgen-independent prostate cancer, colorectal carcinoma, renal cancer, metastatic renal cell carcinoma, soft tissue sarcoma, urinary bladder cancer, and uterine cancer.

Embodiment I-76. The method of embodiment I-71, wherein the disorder is a haematological malignancy.

Embodiment I-77. The method of embodiment I-76, wherein the hematologic malignancy is selected from the group consisting of acute myeloid leukemia, chronic myelogenous leukemia (CML), accelerated CML, CML blast phase (CML-BP), acute lymphoblastic leukemia, chronic lymphocytic leukemia (CLL), Hodgkin's disease, non-Hodgkin's lymphoma, follicular lymphoma, mantle cell lymphoma, B-cell lymphoma, T-cell lymphoma, multiple myeloma, Waldenstrom's macroglobulinemia, myelodysplastic syndromes (MDS), refractory anemia (RA), RA with ringed sideroblasts, RA with excess blasts (RAEB), RAEB in transformation, and a myeloproliferative syndrome.

Embodiment I-78. A method of treating or reducing the effect of a gastrointestinal disease or disorder, the method comprising administration of an effective amount of a compound of any one of embodiments I-1 to I-66, or a pharmaceutically acceptable salt or tautomer thereof, or the pharmaceutical composition of embodiment I-67.

Embodiment I-79. The method of embodiment I-78, wherein the gastrointestinal disorder is IBD, Crohn's disease, or colitis.

Embodiment I-80. A compound of any one of embodiments I-1 to I-66, or a pharmaceutically acceptable salt or tautomer thereof, or the pharmaceutical composition of embodiment I-67 for use in modulating activity of NR2F6 by exposure of NR2F6.

Embodiment I-81. A compound of any one of embodiments I-1 to I-66, or a pharmaceutically acceptable salt or tautomer thereof, or the pharmaceutical composition of embodiment I-67 for use in treating or reducing the effect of a disease or disorder associated with NR2F6 modulation.

Embodiment I-82. Use of a compound of any one of embodiments I-1 to I-66, or a pharmaceutically acceptable salt or tautomer thereof, or the pharmaceutical composition of embodiment I-67, for modulating activity of NR2F6.

Embodiment I-83. Use of a compound of any one of embodiments I-1 to I-66, or a pharmaceutically acceptable salt or tautomer thereof, or the pharmaceutical composition of embodiment I-67, for treating or reducing the effect of a disease or disorder associated with NR2F6 modulation.

Embodiment I-84. Use of a compound of any one of embodiments I-1 to I-66, or a pharmaceutically acceptable salt or tautomer thereof, or the pharmaceutical composition of embodiment I-67, in the manufacture of a medicament for modulating activity of NR2F6.

Embodiment I-85. Use of a compound of any one of embodiments I-1 to I-66, or a pharmaceutically acceptable salt or tautomer thereof, or the pharmaceutical composition of embodiment I-67, in the manufacture of a medicament for treating or reducing the effect of a disease or disorder associated with NR2F6 modulation.

Embodiment II-1. A compound represented by Formula (I-A) or (II-A):

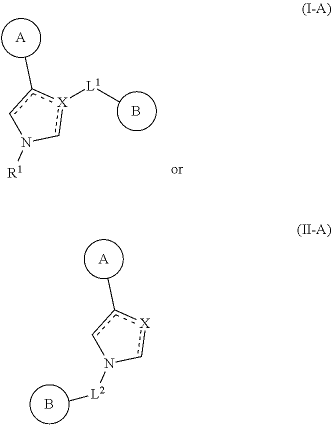

or a pharmaceutically acceptable salt and tautomer thereof, wherein:

each [symbol] independently represents a single bond or a double bond;

X is N, NH, C, CH, or CH₂;

R¹ is H, C₁₋₆alkyl, cycloalkyl, heterocyclyl, —C(O)R^{1a}, —CH₂-aryl, —CH₂-heteroaryl, aryl, or heteroaryl; wherein R^{1a} is C₁₋₆alkyl; and wherein —CH₂-aryl, —CH₂-heteroaryl, aryl, and heteroaryl are optionally substituted with C₁₋₆alkyl or halo;

A is alkyl, cycloalkyl, heterocyclyl, a fused bicyclic aryl, a fused bicyclic heteroaryl, —CH₂-aryl, —CH₂-heteroaryl, aryl, or heteroaryl; wherein the aryl or heteroaryl is optionally substituted with aryl, heteroaryl, —Y^{A}-aryl, or —Y^{A}-heteroaryl; wherein Y^{A} is —O—, —C(O)—, —N(R^{A1})—, S(O)—, or —S(O)₂—; wherein R^{A1} is H or C₁₋₆alkyl;
wherein the fused bicyclic aryl, the fused bicyclic heteroaryl, —CH₂-aryl, —CH₂— heteroaryl, each aryl, and each heteroaryl are optionally substituted with one or more substituents selected from the group consisting of alkyl, halo, haloalkyl, —CN, —N(R^{A})₂, —OH, and —O-alkyl; wherein each R^{A} is independently H or C₁₋₆alkyl;

L¹ is —C(O)—NR^{L1}—, —O—C(S)—NR^{L1}—, —O—C(O)—NR^{L1}—, —NR^{L1}—C(O)—, —NR^{L1}—C(O)—O—, —NH—C(O)—NH—, —NR^{L1}—C(S)—NR^{L1}—, —NR^{L1}—S(O)₂—, —S(O)₂—NR^{L1}—, —CH₂—CH₂—, —CH₂—NR^{L1}—, —NR^{L1}—CH₂—, —CH₂—O—, —O—CH₂—, —O—, —NH—, —C(O)-azetidinyl, —CH₂—NR^{L1}—C(O)—, —C(O)—NR^{L1}—CH₂—, or —C(O)—; wherein each R^{L1} is independently H or C₁₋₆alkyl; and

L² is —C(O)—NR^{L2}—, —S(O)₂—NR^{L2}—, —CH₂—CH₂—, —C(S)—NR^{L2}—, —C(O)—, or —S(O)₂—; wherein each R^{L2} is independently H or C₁₋₆alkyl; and

B is a fused bicyclic aryl, a fused bicyclic heteroaryl, —CH₂-aryl, —CH₂-heteroaryl, aryl, heteroaryl, cycloalkyl, —CH₂-heterocyclyl, or heterocyclyl, wherein the aryl, heteroaryl, cycloalkyl, or heterocyclyl is optionally substituted with aryl, heteroaryl, —Y^{B}-aryl, —Y^{B}— heteroaryl, —Y^{B}-heterocyclyl, or cycloalkyl; wherein Y^{B} is —O—, —CH₂—, —C(O)—, —N(R^{B1})—, —S(O)—, or —S(O)₂—; wherein R^{B1} is H or C₁₋₆alkyl;
wherein the fused bicyclic aryl, the fused bicyclic heteroaryl, —CH₂-aryl, —CH₂— heteroaryl, each aryl, each heteroaryl, each cycloalkyl, —CH₂-heterocyclyl, and each heterocyclyl are optionally substituted with one or more substituents selected from the group consisting of alkyl, halo, haloalkyl, —CN, —N(R^{B2})₂, —OH, —O-alkyl, and oxo;

wherein each R^{B2} is independently H or C₁₋₆alkyl;

wherein when the compound is Formula (I-A); A is phenyl, and L¹ is —C(O)—NH—; then Bis not

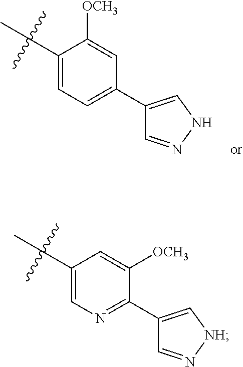 [SMILES: *c1ccc(-c2cn[nH]c2)cc1OC ; "or" ; *c1cnc(-c2cn[nH]c2)c(OC)c1]

wherein when the compound is Formula (I-A); A is a substituted phenyl and B is a substituted phenyl, then L¹ is not —C(O)—NH—, —NH—C(O)—, —NCH₃—C(O)—, or —NH—C(O)—NH—;

wherein when the compound is Formula (I-A); L¹ is —C(O)—NR^{L1}—CH₂— and B is an optionally substituted phenyl, substituted pyridyl, or

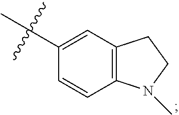 [SMILES: *c1ccc2c(c1)CCN2C]

then A is not substituted phenyl, substituted pyridyl, substituted thiophenyl, substituted thiazolyl, substituted pyrazolyl,

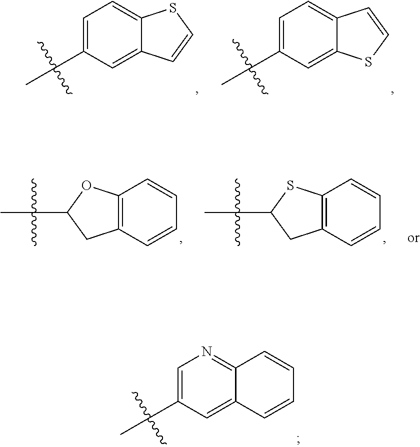 [SMILES: *c1ccc2sccc2c1 ; *c1ccc2ccsc2c1 ; *C1Cc2ccccc2O1 ; *C1Cc2ccccc2S1 ; *c1cnc2ccccc2c1]

wherein when the compound is Formula (I-A); B is optionally substituted —CH₂-aryl and A is optionally substituted aryl; then L¹ is not —C(O)—NH—;

wherein when the compound is Formula (II-A); A is optionally substituted phenyl and B is optionally substituted phenyl, then L¹ is not —C(O)—NCH₃—.

Embodiment II-2. A compound of Formula (I) or (II):

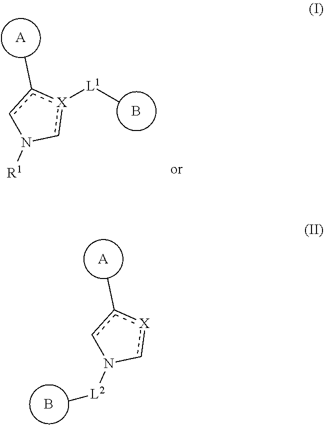

or a pharmaceutically acceptable salt or tautomer thereof, wherein:

each [symbol] independently represents a single bond or a double bond;

X is N, NH, C, CH, or CH₂;

R¹ is H, C₁₋₆alkyl, cycloalkyl, heterocyclyl, —C(O)R^{1a}, —CH₂-aryl, —CH₂-heteroaryl, aryl, or heteroaryl; wherein R^{1a} is C₁₋₆alkyl; and wherein —CH₂-aryl, —CH₂-heteroaryl, aryl, and heteroaryl are optionally substituted with C₁₋₆alkyl or halo;

A is alkyl, cycloalkyl, heterocyclyl, a fused bicyclic aryl, a fused bicyclic heteroaryl, —CH₂-aryl, —CH₂-heteroaryl, aryl, or heteroaryl; wherein the aryl or heteroaryl is optionally substituted with aryl, heteroaryl, —Y^{A}-aryl, or —Y^{A}-heteroaryl; wherein Y^{A} is —O—, —C(O)—, —N(R^{A1})—, —S(O)—, or —S(O)₂—; wherein R^{A1} is H or C₁₋₆alkyl;
wherein the fused bicyclic aryl, the fused bicyclic heteroaryl, —CH₂-aryl, —CH₂— heteroaryl, each aryl, and each heteroaryl are optionally substituted with one or more substituents selected from the group consisting of alkyl, halo, —CN, —N(R^{A})₂, —OH, and —O-alkyl; wherein each R^{A} is independently H or C₁₋₆alkyl;

L¹ is —C(O)—NR^{L1}—, —O—C(S)—NR^{L1}—, —O—C(O)—NR^{L1}—, —NR^{L1}—C(O)—, —NR^{L1}—C(O)—O—, —NH—C(O)—NH—, —NR^{L1}—C(S)—NR^{L1}—, —NR^{L1}—S(O)₂—, —S(O)₂—NR^{L1}—, —CH₂—CH₂—, —CH₂—NR^{L1}—, —NR^{L1}—CH₂—, —CH₂—O—, —O—CH₂—, —O—, —NH—, —C(O)-azetidinyl, —CH₂—NR^{L1}—C(O)—, or —C(O)—NR^{L1}—CH₂—; wherein each R^{L1} is independently H or C₁₋₆alkyl; and

L² is —C(O)—NR^{L2}—, —S(O)₂—NR^{L2}—, —CH₂—CH₂—, —C(S)—NR^{L2}—, —C(O)—, or —S(O)₂—; wherein each R^{L2} is independently H or C₁₋₆alkyl; and

B is a fused bicyclic aryl, a fused bicyclic heteroaryl, —CH₂-aryl, —CH₂-heteroaryl, aryl, heteroaryl, cycloalkyl, or —CH₂-heterocyclyl, wherein the aryl or heteroaryl is optionally substituted with aryl, heteroaryl, —Y^{B}-aryl, or —Y^{B}-heteroaryl; wherein Y^{B} is —O—, —C(O)—, —N(R^{B1})—, —S(O)—, or —S(O)₂—; wherein R^{B1} is H or C₁₋₆alkyl;
wherein the fused bicyclic aryl, the fused bicyclic heteroaryl, —CH₂-aryl, —CH₂— heteroaryl, each aryl, each heteroaryl, cycloalkyl, and —CH₂-heterocyclyl are optionally substituted with one or more substituents selected from the group consisting of alkyl, halo, —CN, N(R^{B2})₂, —OH, and —O-alkyl; wherein each R^{B2} is independently H or C₁₋₆alkyl;

wherein when the compound is Formula (I); A is optionally substituted phenyl or thiophenyl, and L¹ is —C(O)—NH—; then B is not

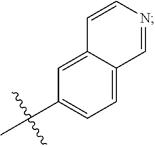 [SMILES: *c1ccc2cnccc2c1]

wherein when the compound is Formula (I); A is a substituted phenyl and B is a substituted phenyl, then L¹ is not —C(O)—NH—, —NH—C(O)—, —NCH₃—C(O)—, or —NH—C(O)—NH—;

wherein when the compound is Formula (I); B is optionally substituted —CH₂-aryl and A is optionally substituted aryl; then L¹ is not —C(O)—NH—;

wherein when the compound is Formula (II); A is optionally substituted phenyl and B is optionally substituted phenyl, then L¹ is not —C(O)—NCH₃—.

Embodiment II-3. A compound of Formula (III):

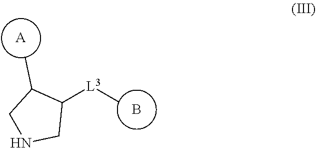

or a pharmaceutically acceptable salt or tautomer thereof, wherein:

A is aryl or 5- to 6-membered heteroaryl, wherein the aryl and heteroaryl are optionally substituted with one or more substituents selected from the group consisting of alkyl, halo, —OH, and —O-alkyl;

L³ is —C(O)—NR^{L3}—, —O—C(S)—NR^{L3}—, —O—C(O)—NR^{L3}—, —NR^{L3}—C(O)—, —NR^{L3}—C(S)—NR^{L3}—, —NR^{L3}—S(O)₂—, —S(O)₂—NR^{L3}—, —CH₂—CH₂—, —CH₂—NR^{L3}—, —NR^{L3}—CH₂—, —CH₂—O—, —O—CH₂—, or —O—; wherein each R^{L3} is independently hydrogen or C₁₋₆alkyl; and

B is a fused bicyclic aryl, a fused bicyclic heteroaryl, —CH₂-aryl, —CH₂-heteroaryl, aryl, or heteroaryl, wherein the aryl or heteroaryl is optionally substituted with aryl or heteroaryl;
wherein the fused bicyclic aryl, the fused bicyclic heteroaryl, —CH₂-aryl, —CH₂— heteroaryl, each aryl, and each heteroaryl are optionally substituted with one or more substituents selected from the group consisting of alkyl, halo, —OH, and —O-alkyl;

wherein when A is optionally substituted phenyl or thiophenyl, and L³ is —C(O)—NH—; then B is not

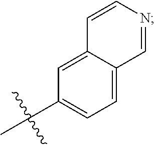 [SMILES: *c1ccc2cnccc2c1]

wherein when A is a substituted phenyl and B is a substituted phenyl, then L³ is not —C(O)—NH—, —NH—C(O)—, —NCH₃—C(O)—, or —NH—C(O)—NH—;

wherein when the compound is Formula (I); B is optionally substituted —CH₂-aryl and A is optionally substituted aryl; then L³ is not —C(O)—NH—.

Embodiment II-4. A compound of Formula (IV):

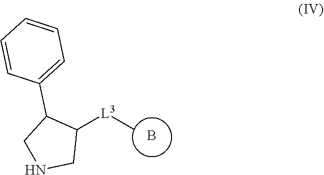

or a pharmaceutically acceptable salt or tautomer thereof, wherein:

L³ is —C(O)—NR^{L3}—, —O—C(S)—NR^{L3}—, —O—C(O)—NR^{L3}—, —NR^{L3}—C(O)—, —NR^{L3}—C(S)—NR^{L3}—, —NR^{L3}—S(O)₂—, —S(O)₂—NR^{L3}—, —CH₂—CH₂—, —CH₂—NR^{L3}—, —NR^{L3}—CH₂—, —CH₂—O—, —O—CH₂—, or —O—; wherein each R^{L3} is independently hydrogen or C₁₋₆alkyl; and

B is a fused bicyclic aryl, a fused bicyclic heteroaryl, —CH₂-aryl, —CH₂-heteroaryl, aryl, or heteroaryl, wherein the aryl or heteroaryl is optionally substituted with aryl or heteroaryl;
wherein the fused bicyclic aryl, the fused bicyclic heteroaryl, —CH₂-aryl, —CH₂-heteroaryl, each aryl, and each heteroaryl are optionally substituted with one or more substituents selected from the group consisting of alkyl, halo, —OH, and —O-alkyl;

wherein when L³ is —C(O)—NH—; then B is not

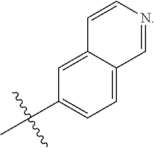 [SMILES: *c1ccc2cnccc2c1]

Embodiment II-5. A compound of Formula (V):

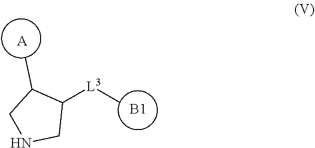

or a pharmaceutically acceptable salt or tautomer thereof, wherein:

A is aryl or 5- to 6-membered heteroaryl, wherein the aryl and heteroaryl are optionally substituted with one or more substituents selected from the group consisting of alkyl, halo, —OH, and —O-alkyl;

L³ is —C(O)—NR^{L3}—, —O—C(S)—NR^{L3}—, —O—C(O)—NR^{L3}—, —NR^{L3}—C(O)—, —NR^{L3}—C(S)—NR^{L3}—, —NR^{L3}—S(O)₂—, —S(O)₂—NR^{L3}—, —CH₂—CH₂—, —CH₂—NR^{L3}—, —NR^{L3}—CH₂—, —CH₂—O—, —O—CH₂—, or —O—; wherein each R^{L3} is independently hydrogen or C₁₋₆alkyl; and

B1 is a fused bicyclic aryl or a fused bicyclic heteroaryl; wherein the fused bicyclic aryl and the fused bicyclic heteroaryl are optionally substituted with one or more substituents selected from the group consisting of alkyl, halo, —OH, and —O-alkyl;

wherein when A is optionally substituted phenyl or thiophenyl, and L³ is —C(O)—NH—; then B is not

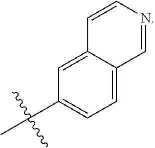 [SMILES: *c1ccc2cnccc2c1]

Embodiment II-6. The compound of Embodiment II-5, or a pharmaceutically acceptable salt or tautomer thereof, wherein B1 is a fused bicyclic aryl.

Embodiment II-7. The compound of Embodiment II-5, or a pharmaceutically acceptable salt or tautomer thereof, wherein B1 is a fused bicyclic heteroaryl.

Embodiment II-8. The compound of Embodiment II-5, or a pharmaceutically acceptable salt or tautomer thereof, wherein B1 is selected from the group consisting of

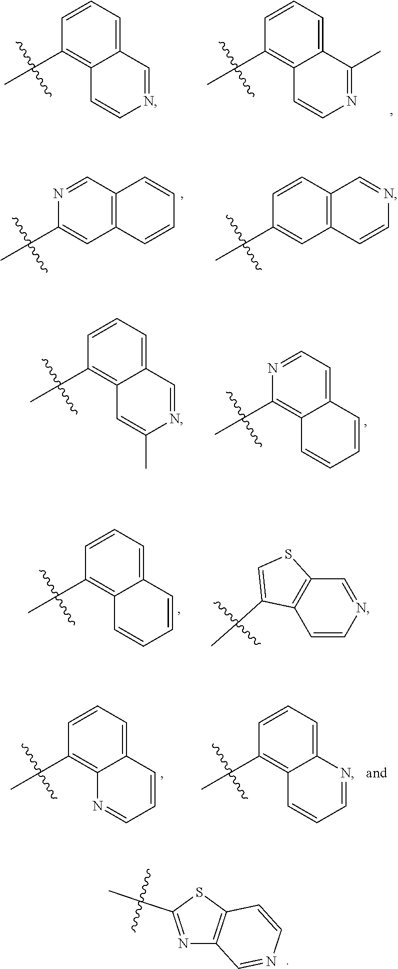

Embodiment II-9. A compound of Formula (VI):

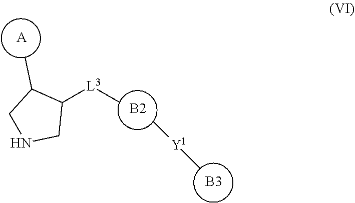

or a pharmaceutically acceptable salt or a tautomer thereof, wherein:

A is aryl or 5- to 6-membered heteroaryl, wherein the aryl and heteroaryl are optionally substituted with one or more substituents selected from the group consisting of alkyl, halo, —OH, and —O-alkyl;

L³ is —C(O)—NR^{L3}—, —O—C(S)—NR^{L3}—, —O—C(O)—NR^{L3}—, —NR^{L3}—C(O)—, —NR^{L3}—C(S)—NR^{L3}—, —NR^{L3}—S(O)₂—, —S(O)₂—NR^{L3}—, —CH₂—CH₂—, —CH₂—NR^{L3}—, —NR^{L3}—CH₂—, —CH₂—O—, —O—CH₂—, or —O—; wherein each R^{L3} is independently hydrogen or C₁₋₆alkyl; and

B2 is monocyclic aryl or monocyclic heteroaryl; wherein the aryl and heteroaryl are optionally substituted with one or more substituents selected from the group consisting of alkyl, halo, —OH, and —O-alkyl;

Y¹ is absent, —O—, —C(O)—, —N(R^{Y})—, —S(O)—, or —S(O)₂—; wherein R^{Y} is H or C₁₋₆alkyl; and

B3 is monocyclic aryl or monocyclic heteroaryl; wherein the aryl and heteroaryl are optionally substituted with one or more substituents selected from the group consisting of alkyl, halo, —OH, and —O-alkyl.

Embodiment II-10. The compound of Embodiment II-9, or a pharmaceutically acceptable salt or tautomer thereof, wherein B2 is monocyclic aryl.

Embodiment II-11. The compound of Embodiment II-9, or a pharmaceutically acceptable salt or tautomer thereof, wherein B2 is monocyclic heteroaryl.

Embodiment II-12. The compound of Embodiment II-9, or a pharmaceutically acceptable salt or tautomer thereof, wherein B3 is monocyclic aryl.

Embodiment II-13. The compound of Embodiment II-9, or a pharmaceutically acceptable salt or tautomer thereof, wherein B3 is monocyclic heteroaryl.

Embodiment II-14. The compound of Embodiment II-9, or a pharmaceutically acceptable salt or tautomer thereof, wherein

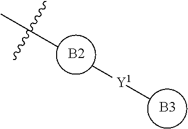

is selected from the group consisting of

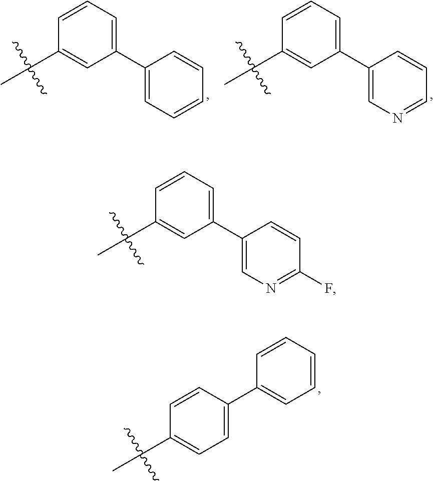 [SMILES: *c1cccc(-c2ccccc2)c1 ; *c1cccc(-c2cccnc2)c1 ; *c1cccc(-c2ccc(F)nc2)c1 ; *c1ccc(-c2ccccc2)cc1]

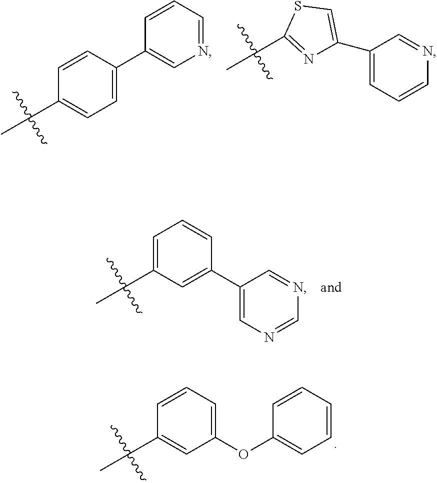 [SMILES: *c1ccc(-c2cccnc2)cc1 ; *c1nc(-c2cccnc2)cs1 ; *c1cccc(-c2cncnc2)c1 ; "and" ; *c1cccc(Oc2ccccc2)c1]

Embodiment II-15. A compound of Formula (VII):

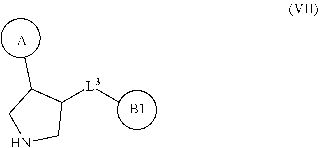

or a pharmaceutically acceptable salt or tautomer thereof, wherein:

A is aryl or 5- to 6-membered heteroaryl, wherein the aryl and heteroaryl are optionally substituted with one or more substituents selected from the group consisting of alkyl, halo, —OH, and —O-alkyl;

L³ is —C(O)—NR^{L3}—, —O—C(S)—NR^{L3}—, —O—C(O)—NR^{L3}—, —NR^{L3}—C(O)—, —NR^{L3}—C(S)—NR^{L3}—, —NR^{L3}—S(O)₂—, —S(O)₂—NR^{L3}—, —CH₂—CH₂—, —CH₂—NR^{L3}—, —NR^{L3}—CH₂—, —CH₂—O—, —O—CH₂—, or —O—; wherein each R^{L3} is independently hydrogen or C₁₋₆alkyl; and

B1 is a fused bicyclic aryl or a fused bicyclic heteroaryl; wherein the fused bicyclic aryl and the fused bicyclic heteroaryl are optionally substituted with one or more substituents selected from the group consisting of alkyl, halo, —OH, and —O-alkyl;

wherein when A is optionally substituted phenyl or thiophenyl, and L³ is —C(O)—NH—; then B is not

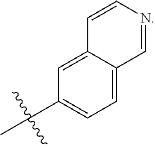 [SMILES: *c1ccc2cnccc2c1]

Embodiment II-16. The compound of Embodiment II-15, or a pharmaceutically acceptable salt or tautomer thereof, wherein B4 is —CH₂-aryl.

Embodiment II-17. The compound of Embodiment II-15, or a pharmaceutically acceptable salt or tautomer thereof, wherein B4 is —CH₂-heteroaryl.

Embodiment II-18. The compound of Embodiment II-15, or a pharmaceutically acceptable salt or tautomer thereof, wherein B4 is selected from the group consisting of

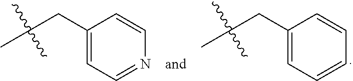 [SMILES: *Cc1ccncc1 ; "and" ; *Cc1ccccc1 ; "."]

Embodiment II-19. The compound of Embodiment II-1 or II-2, or a pharmaceutically acceptable salt or tautomer thereof, wherein

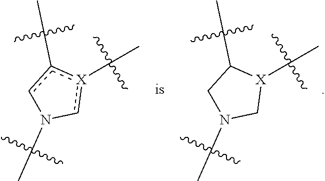

Embodiment II-20. The compound of Embodiment II-1 or II-2, or a pharmaceutically acceptable salt or tautomer thereof, wherein

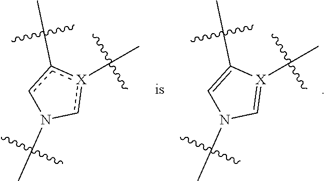

Embodiment II-21 The compound of Embodiment II-1 or II-2, or a pharmaceutically acceptable salt or tautomer thereof, wherein

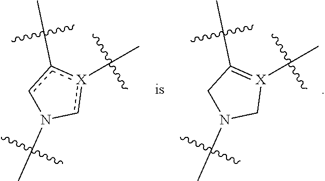

Embodiment II-22. The compound of any one of Embodiments II-1 to II-2 and II-19 to II-21, or a pharmaceutically acceptable salt or tautomer thereof, wherein X is N or NH.

Embodiment II-23. The compound of any one of Embodiments II-1 to II-2 and II-19 to II-21, or a pharmaceutically acceptable salt or tautomer thereof, wherein X is C, CH, or CH₂.

Embodiment II-24. The compound of any one of Embodiments II-1 to II-2 and II-19 to II-23, or a pharmaceutically acceptable salt or tautomer thereof, wherein R¹ is H.

Embodiment II-25. The compound of any one of Embodiments II-1 to II-2 and II-19 to II-23, or a pharmaceutically acceptable salt or tautomer thereof, wherein R¹ is C₁₋₆alkyl.

Embodiment II-26. The compound of any one of Embodiments II-1 to II-2 and II-19 to II-23, or a pharmaceutically acceptable salt or tautomer thereof, wherein R¹ is cycloalkyl.

Embodiment II-27. The compound of any one of Embodiments II-1 to II-2 and II-19 to II-23, or a pharmaceutically acceptable salt or tautomer thereof, wherein R¹ is heterocyclyl.

Embodiment II-28. The compound of any one of Embodiments II-1 to II-2 and II-19 to II-23, or a pharmaceutically acceptable salt or tautomer thereof, wherein R¹ is —C(O)R^{1a}.

Embodiment II-29. The compound of any one of Embodiments II-1 to II-2 and II-19 to II-23, or a pharmaceutically acceptable salt or tautomer thereof, wherein R¹ is —CH₂-aryl.

Embodiment II-30. The compound of any one of Embodiments II-1 to II-29, or a pharmaceutically acceptable salt thereof, wherein A is aryl.

Embodiment II-31. The compound of Embodiment II-30, or a pharmaceutically acceptable salt or tautomer thereof, wherein the aryl is substituted with one or more substituents selected from the group consisting of alkyl, halo, —OH, and —O-alkyl.

Embodiment II-32. The compound of any one of Embodiments II-1 to II-29, or a pharmaceutically acceptable salt or tautomer thereof, wherein A is 5- to 6-membered heteroaryl.

Embodiment II-33. The compound of Embodiment II-32, or a pharmaceutically acceptable salt or tautomer thereof, wherein the heteroaryl is substituted with one or more substituents selected from the group consisting of alkyl, halo, —OH, and —O-alkyl.

Embodiment II-34. The compound of any one of Embodiments II-1 to II-29, or a pharmaceutically acceptable salt or tautomer thereof, wherein A is alkyl.

Embodiment II-35. The compound of any one of Embodiments II-1 to II-29, or a pharmaceutically acceptable salt or tautomer thereof, wherein A is cycloalkyl.

Embodiment II-36. The compound of any one of Embodiments II-1 to II-29, or a pharmaceutically acceptable salt or tautomer thereof, wherein A is heterocyclyl.

Embodiment II-37. The compound of any one of Embodiments II-1 to II-29, or a pharmaceutically acceptable salt or tautomer thereof, wherein A is a fused bicyclic aryl or a fused bicyclic heteroaryl.

Embodiment II-38. The compound of any one of Embodiments II-1 to II-29, or a pharmaceutically acceptable salt or tautomer thereof, wherein A is —CH₂-aryl or —CH₂— heteroaryl.

Embodiment II-39. The compound of any one of Embodiments II-1 to II-2 and II-18 to II-38, or a pharmaceutically acceptable salt or tautomer thereof, wherein L¹ is —C(O)—NR^{L1}—.

Embodiment II-40. The compound of any one of Embodiments II-1 to II-2 and II-18 to II-38, or a pharmaceutically acceptable salt or tautomer thereof, wherein L¹ is —O—C(S)—NR^{L1}—.

Embodiment II-41. The compound of any one of Embodiments II-1 to II-2 and II-18 to II-38, or a pharmaceutically acceptable salt or tautomer thereof, wherein L¹ is —O—C(O)—NR^{L1}—.

Embodiment II-42. The compound of any one of Embodiments II-1 to II-2 and II-18 to II-38, or a pharmaceutically acceptable salt or tautomer thereof, wherein L¹ is —NR^{L1}—C(S)—NR^{L1}—.

Embodiment II-43. The compound of any one of Embodiments II-1 to II-2 and II-18 to II-38, or a pharmaceutically acceptable salt or tautomer thereof, wherein L¹ is —O—.

Embodiment II-44. The compound of any one of Embodiments II-1 to II-2 and II-18 to II-38, or a pharmaceutically acceptable salt or tautomer thereof, wherein L¹ is —NR^{L1}—C(O)—, —NR^{L1}—C(O)—O—, —NH—C(O)—NH—, —NR^{L1}—S(O)₂— or —S(O)₂—NR^{L1}—.

Embodiment II-45. The compound of any one of Embodiments II-1 to II-2 and II-18 to II-38, or a pharmaceutically acceptable salt or tautomer thereof, wherein L¹ is —CH₂—CH₂—, —CH₂—NR^{L1}—, —NR^{L1}—CH₂—, —CH₂—O—, —O—CH₂—, —NH—, or —C(O)-azetidinyl.

Embodiment II-46. The compound of any one of Embodiments II-1 to II-2 and II-18 to II-38, or a pharmaceutically acceptable salt or tautomer thereof, wherein L² is —C(O)—NR^{L2}—.

Embodiment II-47. The compound of any one of Embodiments II-1 to II-2 and II-18 to II-38, or a pharmaceutically acceptable salt or tautomer thereof, wherein L² is —S(O)₂—NR^{L2}— or —CH₂—CH₂—.

Embodiment II-48. The compound of any one of Embodiments II-3 to II-18 and II-30 to II-38, or a pharmaceutically acceptable salt or tautomer thereof, wherein L³ is —C(O)—NR^{L3}—.

Embodiment II-49. The compound of any one of Embodiments II-3 to II-18 and II-30 to II-38, or a pharmaceutically acceptable salt or tautomer thereof, wherein L³ is —O—C(S)—NR^{L3}—.

Embodiment II-50. The compound of any one of Embodiments II-3 to II-18 and II-30 to II-38, or a pharmaceutically acceptable salt or tautomer thereof, wherein L³ is —O—C(O)—NR^{L3}—.

Embodiment II-51. The compound of any one of Embodiments II-3 to II-18 and II-30 to II-38, or a pharmaceutically acceptable salt or tautomer thereof, wherein L³ is —NR^{L3}—C(S)—NR^{L3}—.

Embodiment II-52. The compound of any one of Embodiments II-3 to II-18 and II-30 to II-38, or a pharmaceutically acceptable salt or tautomer thereof, wherein L³ is —NR^{L3}—C(O)—, —NR^{L3}—S(O)₂—, —S(O)₂—NR^{L3}—, —CH₂—CH₂—, —CH₂—NR^{L3}—, or —NR^{L3}—CH₂—.

Embodiment II-53. The compound of any one of Embodiments II-3 to II-18 and II-30 to II-38, or a pharmaceutically acceptable salt or tautomer thereof, wherein L³ is —CH₂—O—, —O—CH₂—, or —O—.

Embodiment II-54. The compound of any one of Embodiments II-1 to II-4 and II-19 to II-53, or a pharmaceutically acceptable salt or tautomer thereof, wherein B is a fused bicyclic aryl.

Embodiment II-55. The compound of any one of Embodiments II-1 to II-4 and II-19 to II-53, or a pharmaceutically acceptable salt or tautomer thereof, wherein B is a fused bicyclic heteroaryl.

Embodiment II-56. The compound of any one of Embodiments II-1 to II-4 and II-19 to II-53, or a pharmaceutically acceptable salt or tautomer thereof, wherein B is selected from the group consisting of

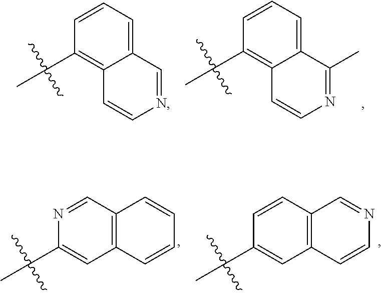 [SMILES: *c1cccc2cnccc12 ; *c1cccc2c(C)nccc12 ; *c1cc2ccccc2cn1 ; *c1ccc2cnccc2c1]

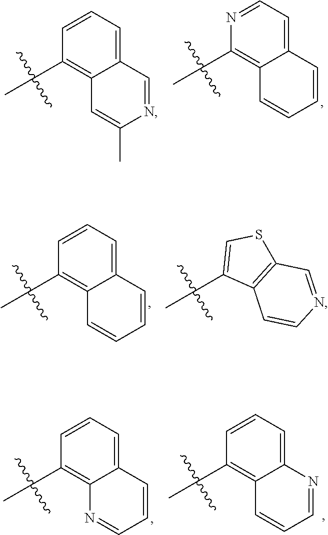 [SMILES: *c1cccc2cnc(C)cc12 ; *c1nccc2ccccc12 ; *c1cccc2ccccc12 ; *c1csc2cnccc12 ; *c1cccc2cccnc12 ; *c1cccc2ncccc12]

and

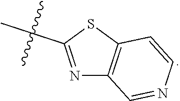 [SMILES: *c1nc2cnccc2s1]

Embodiment II-57. The compound of any one of Embodiments II-1 to II-4 and II-19 to II-53, or a pharmaceutically acceptable salt or tautomer thereof, wherein B is —CH₂-aryl.

Embodiment II-58. The compound of any one of Embodiments II-1 to II-4 and II-19 to II-53, or a pharmaceutically acceptable salt or tautomer thereof, wherein B is —CH₂-heteroaryl.

Embodiment II-59. The compound of any one of Embodiments II-1 to II-4 and II-19 to II-53, or a pharmaceutically acceptable salt or tautomer thereof, wherein B is selected from the group consisting of

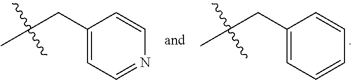 [SMILES: *Cc1ccncc1 ; *Cc1ccccc1]

Embodiment II-60. The compound of any one of Embodiments II-1 to II-4 and II-19 to II-52, or a pharmaceutically acceptable salt or tautomer thereof, wherein B is aryl.

Embodiment II-61. The compound of any one of Embodiments II-1 to II-4 and II-19 to II-52, or a pharmaceutically acceptable salt or tautomer thereof, wherein B is aryl substituted with aryl or heteroaryl.

Embodiment II-62. The compound of any one of Embodiments II-1 to II-4 and II-19 to II-52, or a pharmaceutically acceptable salt or tautomer thereof, wherein B is heteroaryl.

Embodiment II-63. The compound of any one of Embodiments II-1 to II-4 and II-19 to II-52, or a pharmaceutically acceptable salt or tautomer thereof, wherein B is heteroaryl substituted with aryl or heteroaryl.

Embodiment II-64. The compound of any one of Embodiments II-1 to II-4 and II-19 to II-52, or a pharmaceutically acceptable salt or tautomer thereof, wherein B is selected from the group consisting of

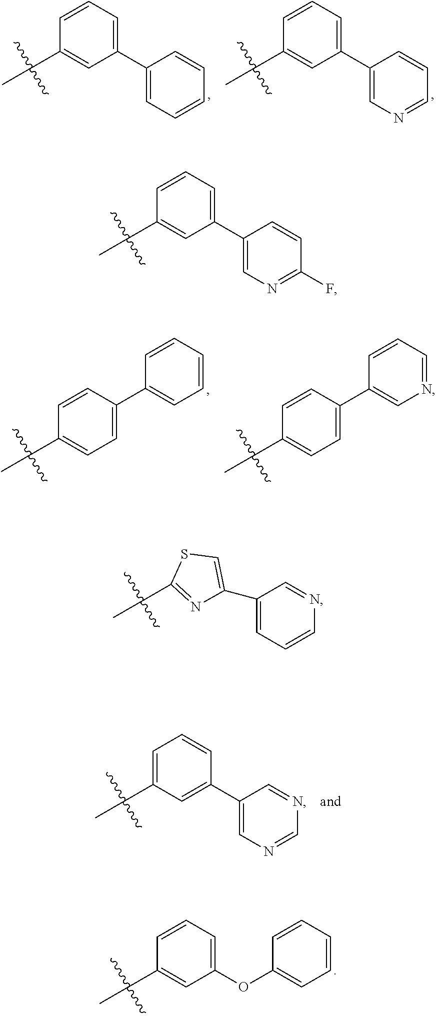

Embodiment II-65. The compound of any one of Embodiments II-1 to II-4 and II-19 to II-52, or a pharmaceutically acceptable salt or tautomer thereof, wherein B is cycloalkyl.

Embodiment II-66. The compound of any one of Embodiments II-1 to II-4 and II-19 to II-52, or a pharmaceutically acceptable salt or tautomer thereof, wherein B is cyclocyclyl substituted with aryl, heteroaryl, —Y^{B}-aryl, —Y^{B}-heteroaryl.

Embodiment II-67. The compound of any one of Embodiments II-1 to II-4 and II-19 to II-52, or a pharmaceutically acceptable salt or tautomer thereof, wherein B is —CH₂— heterocyclyl.

Embodiment II-68. The compound of any one of Embodiments II-1 to II-4 and II-19 to II-52, or a pharmaceutically acceptable salt or tautomer thereof, wherein B is heterocyclyl.

Embodiment II-69. The compound of any one of Embodiments II-1 to II-4 and II-19 to II-52, or a pharmaceutically acceptable salt or tautomer thereof, wherein B is heterocyclyl substituted with aryl or heteroaryl.

Embodiment II-70. A compound, or a pharmaceutically acceptable salt or tautomer thereof, selected from the group consisting of

| Compound | |
|---|---|
| No. | Structure |
| I-1 | 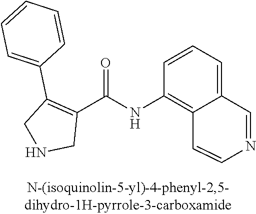 [SMILES: O=C(Nc1cccc2cnccc12)C1=C(c2ccccc2)CNC1] |
| I-2 | 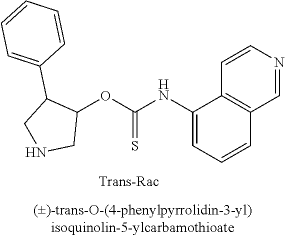 [SMILES: S=C(Nc1cccc2cnccc12)OC1CNCC1c1ccccc1] |
| I-3 | 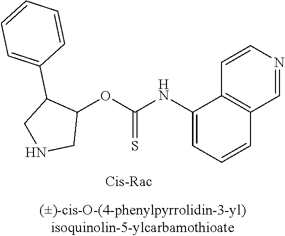 [SMILES: S=C(Nc1cccc2cnccc12)OC1CNCC1c1ccccc1] |
| I-4 | 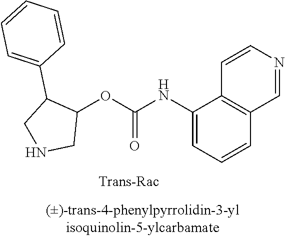 [SMILES: O=C(Nc1cccc2cnccc12)OC1CNCC1c1ccccc1] |
| I-5 | 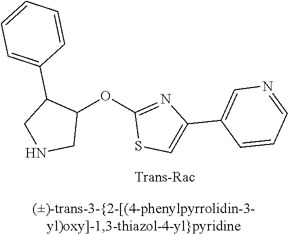 [SMILES: c1ccc(C2CNCC2Oc2nc(-c3cccnc3)cs2)cc1] |
| I-6 | 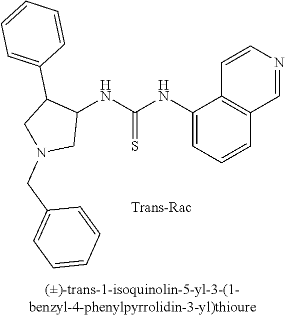 [SMILES: S=C(Nc1cccc2cnccc12)NC1CN(Cc2ccccc2)CC1c1ccccc1] |
| I-7 | 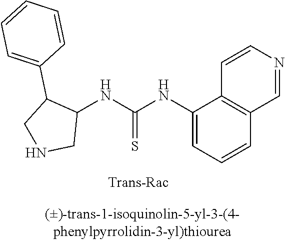 [SMILES: S=C(Nc1cccc2cnccc12)NC1CNCC1c1ccccc1] |
| I-8 | 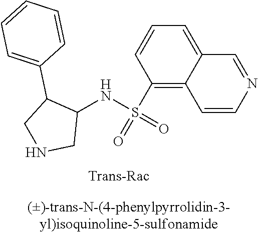 [SMILES: O=S(=O)(NC1CNCC1c1ccccc1)c1cccc2cnccc12] |
| I-9 | 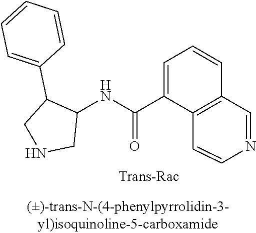 [SMILES: O=C(NC1CNCC1c1ccccc1)c1cccc2cnccc12] |
| I-10 | 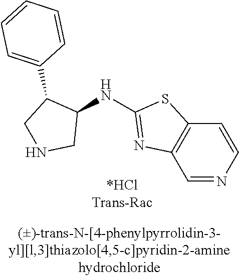 [SMILES: c1ccc([C@H]2CNC[C@@H]2Nc2nc3cnccc3s2)cc1] |
| I-11 | 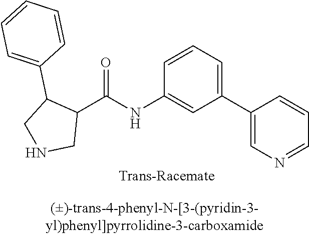 [SMILES: O=C(Nc1cccc(-c2cccnc2)c1)C1CNCC1c1ccccc1] |
| I-12 | 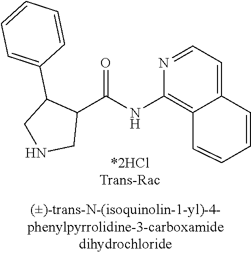 [SMILES: O=C(Nc1nccc2ccccc12)C1CNCC1c1ccccc1] |
| I-13 | 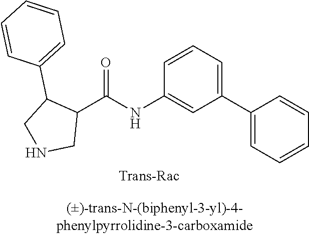 [SMILES: O=C(Nc1cccc(-c2ccccc2)c1)C1CNCC1c1ccccc1] |
| I-14 | 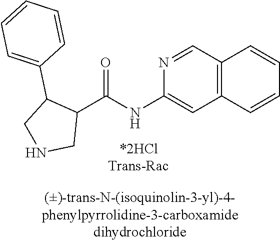 [SMILES: O=C(Nc1cc2ccccc2cn1)C1CNCC1c1ccccc1] |
| I-15 | 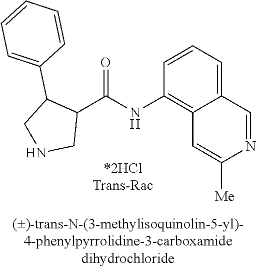 [SMILES: Cc1cc2c(NC(=O)C3CNCC3c3ccccc3)cccc2cn1] |
| I-16 | 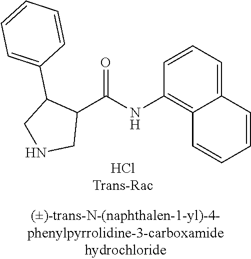 [SMILES: O=C(Nc1cccc2ccccc12)C1CNCC1c1ccccc1] |
| I-17 | 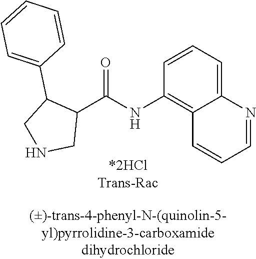 [SMILES: O=C(Nc1cccc2ncccc12)C1CNCC1c1ccccc1] |
| I-18 | 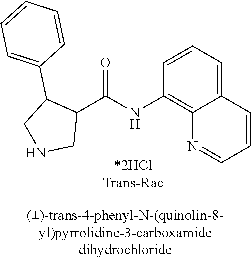 [SMILES: O=C(Nc1cccc2cccnc12)C1CNCC1c1ccccc1] |
| I-19 | 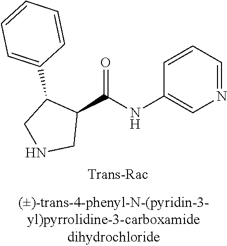 [SMILES: O=C(Nc1cccnc1)[C@H]1CNC[C@@H]1c1ccccc1] |
| I-20 | 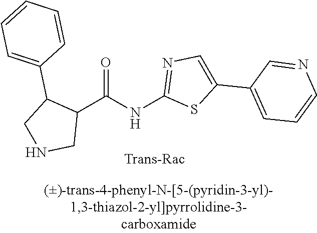 [SMILES: O=C(Nc1ncc(-c2cccnc2)s1)C1CNCC1c1ccccc1] |
| I-21 | 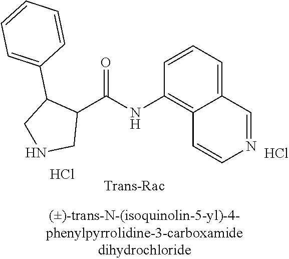 [SMILES: O=C(Nc1cccc2cnccc12)C1CNCC1c1ccccc1 ; Cl ; Cl] |
| I-22 | 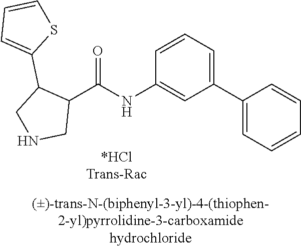 [SMILES: O=C(Nc1cccc(-c2ccccc2)c1)C1CNCC1c1cccs1] |
| I-23 | 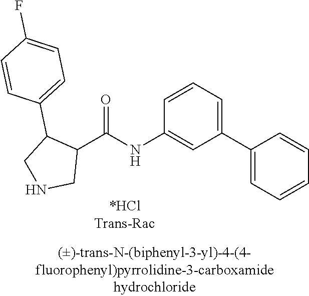 [SMILES: O=C(Nc1cccc(-c2ccccc2)c1)C1CNCC1c1ccc(F)cc1] |
| I-24 | 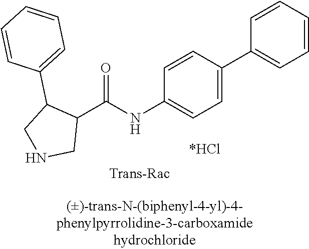 [SMILES: O=C(Nc1ccc(-c2ccccc2)cc1)C1CNCC1c1ccccc1] |
| I-25 | 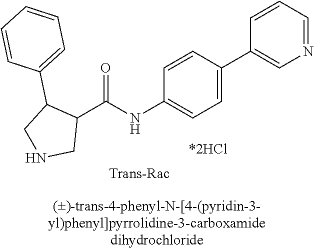 [SMILES: O=C(Nc1ccc(-c2cccnc2)cc1)C1CNCC1c1ccccc1] |
| I-26 | 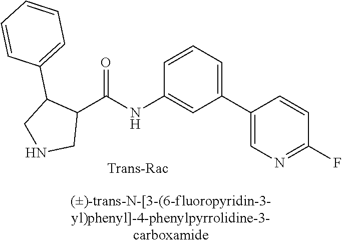 [SMILES: O=C(Nc1cccc(-c2ccc(F)nc2)c1)C1CNCC1c1ccccc1] |
| I-27 | 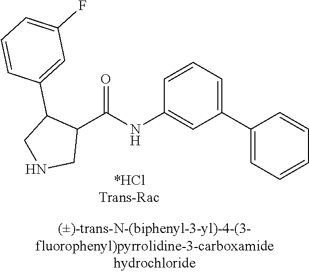 [SMILES: O=C(Nc1cccc(-c2ccccc2)c1)C1CNCC1c1cccc(F)c1] |
| I-28 | 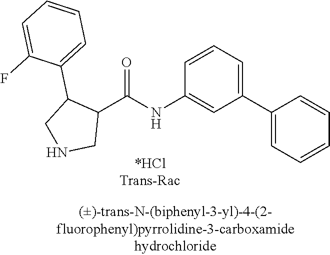 [SMILES: O=C(Nc1cccc(-c2ccccc2)c1)C1CNCC1c1ccccc1F] |
| I-29 | 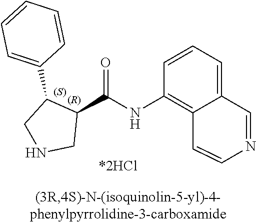 |
| I-30 | 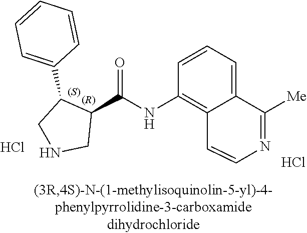 |
| I-31 | 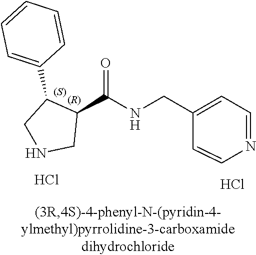 |
| I-32 | 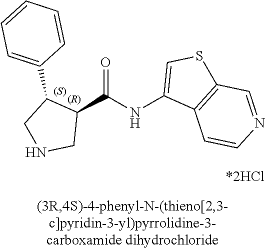 |
| I-33 | 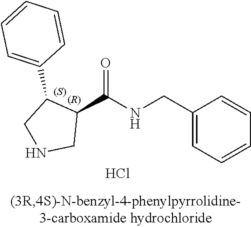 |
| I-34 | 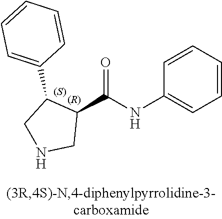 |
| I-35 | 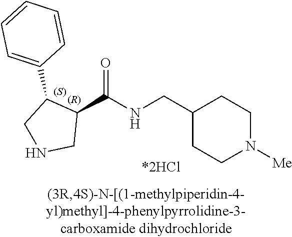 |
| I-36 | 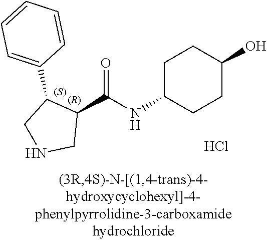 |
| I-37 | 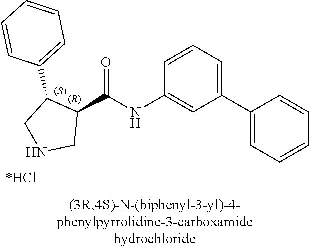 |
| I-38 | 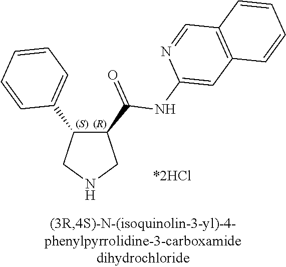 |
| I-39 | 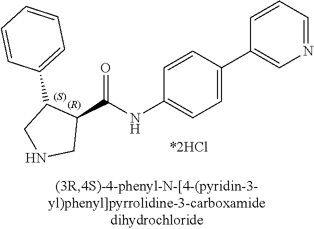 |
| I-40 | 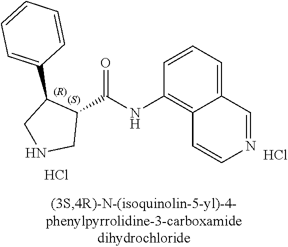 |
| I-41 | 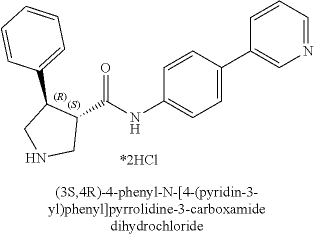 |
| I-42 | 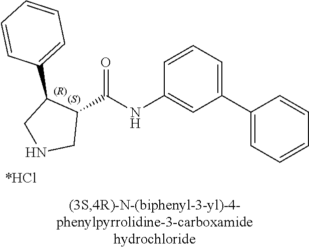 |
| I-43 | 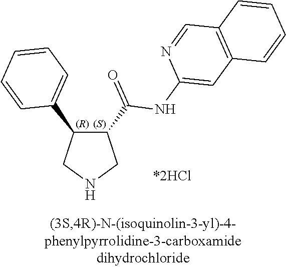 |
| I-44 | 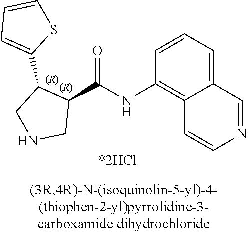 |
| I-45 | 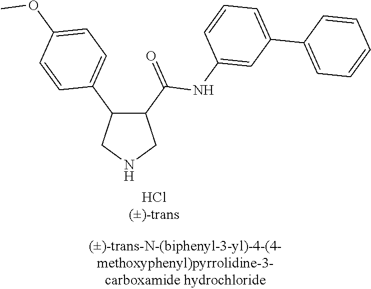 [SMILES: COc1ccc(C2CNCC2C(=O)Nc2cccc(-c3ccccc3)c2)cc1] |
| I-46 | 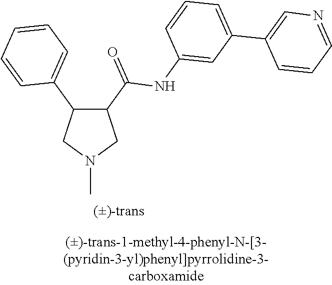 [SMILES: CN1CC(C(=O)Nc2cccc(-c3cccnc3)c2)C(c2ccccc2)C1] |
| I-47 | 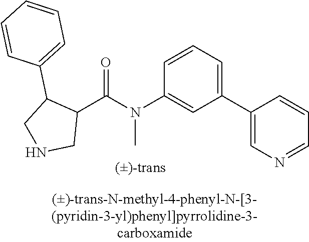 [SMILES: CN(C(=O)C1CNCC1c1ccccc1)c1cccc(-c2cccnc2)c1] |
| I-48 | 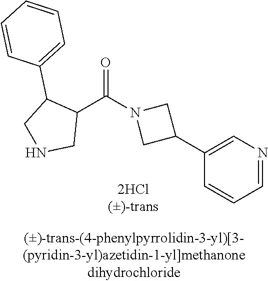 [SMILES: O=C(C1CNCC1c1ccccc1)N1CC(c2cccnc2)C1] |
| I-49 | 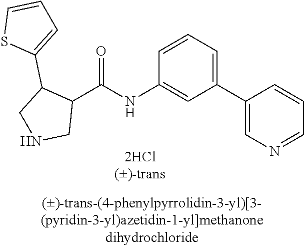 [SMILES: O=C(Nc1cccc(-c2cccnc2)c1)C1CNCC1c1cccs1] |
| I-50 | 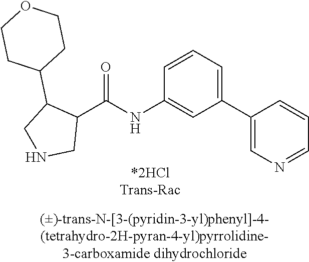 [SMILES: O=C(Nc1cccc(-c2cccnc2)c1)C1CNCC1C1CCOCC1] |
| I-51 | 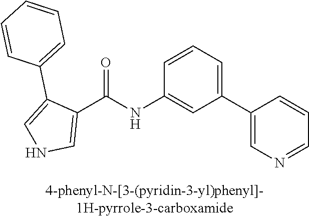 [SMILES: O=C(Nc1cccc(-c2cccnc2)c1)c1c[nH]cc1-c1ccccc1] |
| I-52 | 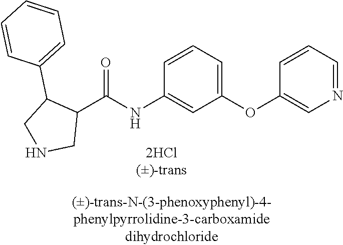 [SMILES: O=C(Nc1cccc(Oc2cccnc2)c1)C1CNCC1c1ccccc1] |
| I-53 | 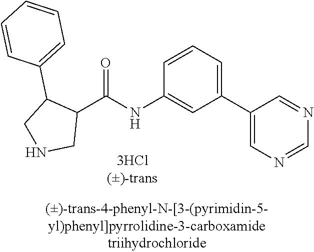 [SMILES: O=C(Nc1cccc(-c2cncnc2)c1)C1CNCC1c1ccccc1] |
| I-54 | 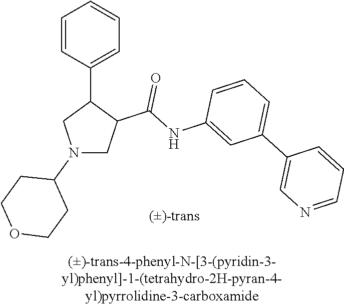 [SMILES: O=C(Nc1cccc(-c2cccnc2)c1)C1CN(C2CCOCC2)CC1c1ccccc1] |
| I-55 | 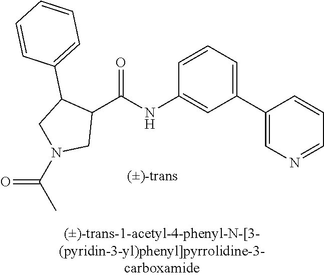 [SMILES: CC(=O)N1CC(C(=O)Nc2cccc(-c3cccnc3)c2)C(c2ccccc2)C1] |
| I-56 | 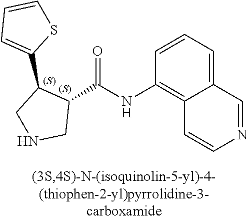 |
| I-57 | 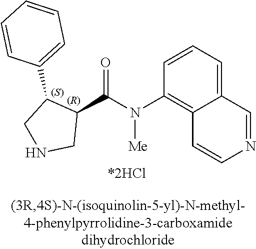 |
| I-58 | 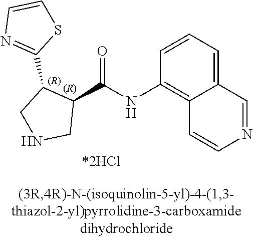 |
| I-59 | 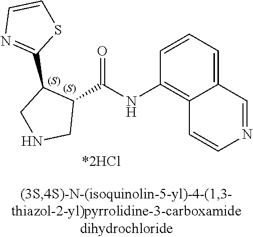 |

Embodiment II-71. A compound, or a pharmaceutically acceptable salt or tautomer thereof, selected from the group consisting of

| Compound | |
|---|---|
| No. | Structure |
| I-60 | 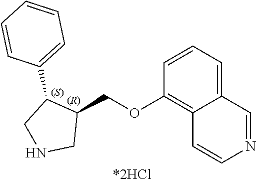 |
| I-61 | 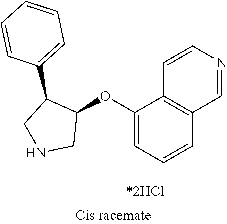 [SMILES: c1ccc([C@@H]2CNC[C@@H]2Oc2cccc3cnccc23)cc1] |
| I-62 | 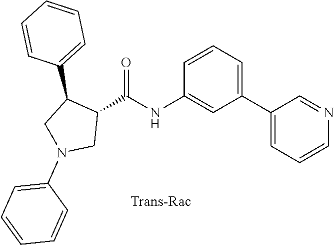 [SMILES: O=C(Nc1cccc(-c2cccnc2)c1)[C@@H]1CN(c2ccccc2)C[C@H]1c1ccccc1] |
| I-63 | 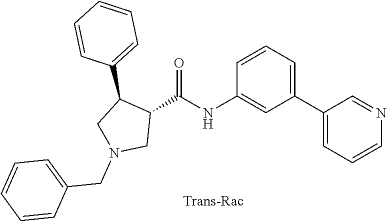 [SMILES: O=C(Nc1cccc(-c2cccnc2)c1)[C@@H]1CN(Cc2ccccc2)C[C@H]1c1ccccc1] |
| I-64 | 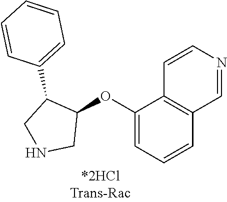 [SMILES: c1ccc([C@H]2CNC[C@@H]2Oc2cccc3cnccc23)cc1] |
| I-65 | 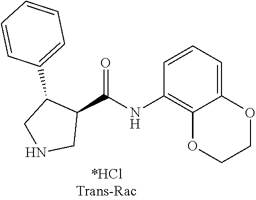 [SMILES: O=C(Nc1cccc2c1OCCO2)[C@H]1CNC[C@@H]1c1ccccc1] |
| I-66 | 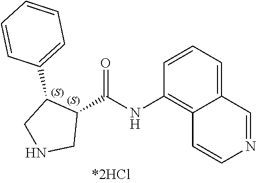 |
| I-67 | 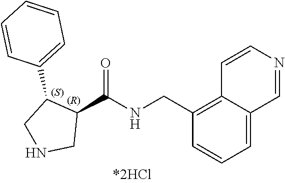 |
| I-68 | 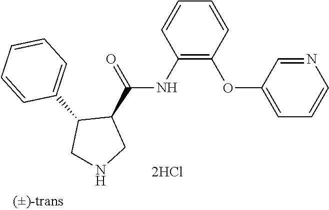 [SMILES: O=C(Nc1ccccc1Oc1cccnc1)[C@H]1CNC[C@@H]1c1ccccc1] |
| I-69 | 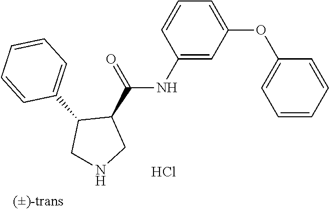 [SMILES: O=C(Nc1cccc(Oc2ccccc2)c1)[C@H]1CNC[C@@H]1c1ccccc1 ; Cl] |
| I-70 | 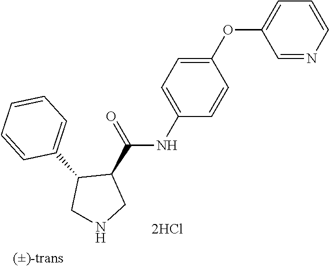 [SMILES: O=C(Nc1ccc(Oc2cccnc2)cc1)[C@H]1CNC[C@@H]1c1ccccc1] |
| I-71 | 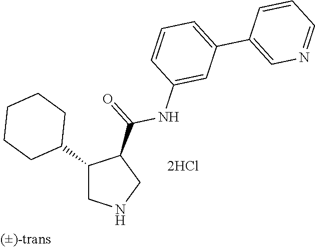 [SMILES: O=C(Nc1cccc(-c2cccnc2)c1)[C@H]1CNC[C@@H]1C1CCCCC1] |
| I-72 | 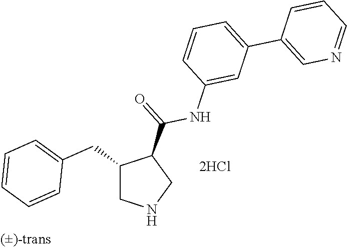 [SMILES: O=C(Nc1cccc(-c2cccnc2)c1)[C@H]1CNC[C@@H]1Cc1ccccc1] |
| I-73 | 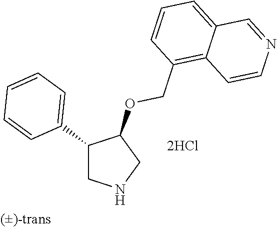 [SMILES: c1ccc([C@H]2CNC[C@@H]2OCc2cccc3cnccc23)cc1] |
| I-74 | 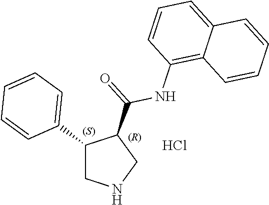 |
| I-75 | 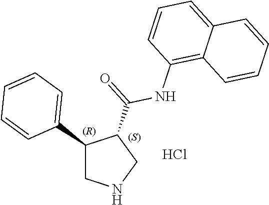 |
| I-76 | 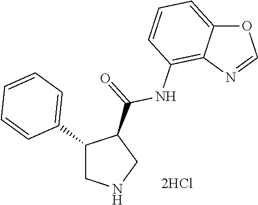 [SMILES: O=C(Nc1cccc2ocnc12)[C@H]1CNC[C@@H]1c1ccccc1 ; "2HCl"] |

Embodiment II-72. A compound, or a pharmaceutically acceptable salt or tautomer thereof, selected from the group consisting of

| Compound | |
|---|---|
| No. | Structure |
| I-77 | 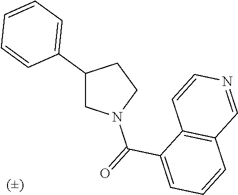 [SMILES: O=C(c1cccc2cnccc12)N1CCC(c2ccccc2)C1] |
| I-78 | 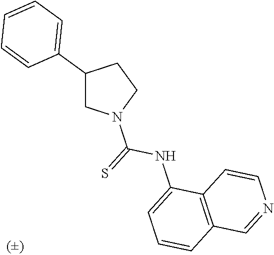 [SMILES: S=C(Nc1cccc2cnccc12)N1CCC(c2ccccc2)C1] |
| I-79 | 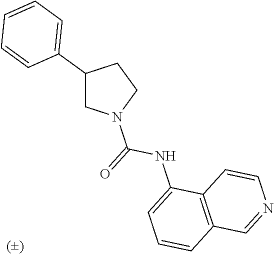 [SMILES: O=C(Nc1cccc2cnccc12)N1CCC(c2ccccc2)C1] |
| I-80 | 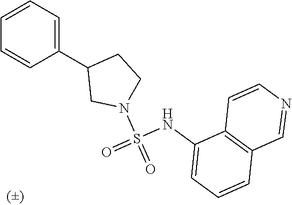 [SMILES: O=S(=O)(Nc1cccc2cnccc12)N1CCC(c2ccccc2)C1] |

Embodiment II-73. A compound, or a pharmaceutically acceptable salt or tautomer thereof, selected from the group consisting of

| Com- | |
|---|---|
| pound | |
| No. | Structure |
| I-81 | 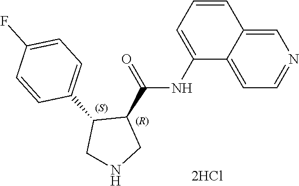 |
| I-82 | 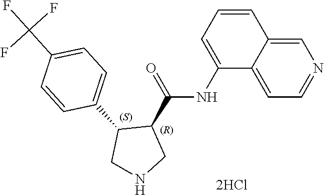 |
| I-83 | 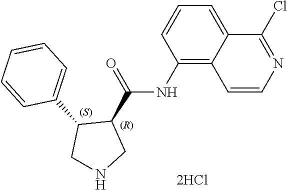 |
| I-84 | 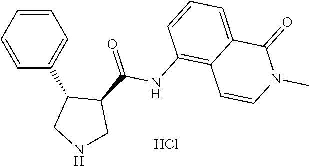 [SMILES: Cn1ccc2c(NC(=O)[C@H]3CNC[C@@H]3c3ccccc3)cccc2c1=O ; Cl] |
| I-85 | 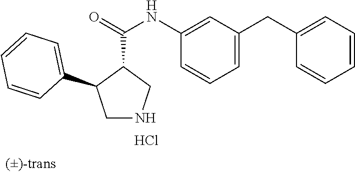 [SMILES: O=C(Nc1cccc(Cc2ccccc2)c1)[C@@H]1CNC[C@H]1c1ccccc1 ; Cl] |
| I-86 | 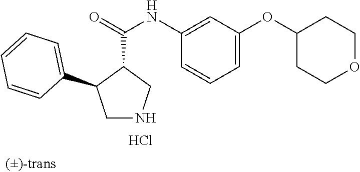 [SMILES: O=C(Nc1cccc(OC2CCOCC2)c1)[C@@H]1CNC[C@H]1c1ccccc1 ; Cl] |
| I-87 | 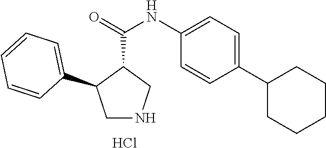 [SMILES: O=C(Nc1ccc(C2CCCCC2)cc1)[C@@H]1CNC[C@H]1c1ccccc1 ; Cl] |
| I-88 | 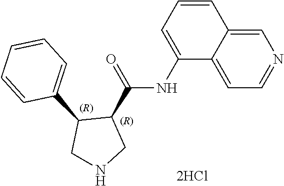 |
| I-89 | 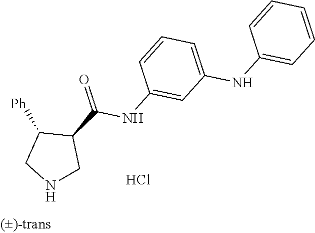 |
| I-90 | 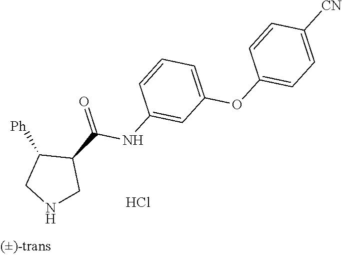 [SMILES: N#Cc1ccc(Oc2cccc(NC(=O)[C@H]3CNC[C@@H]3c3ccccc3)c2)cc1 ; Cl] |
| I-91 | 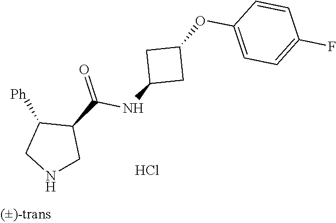 |
| I-92 | 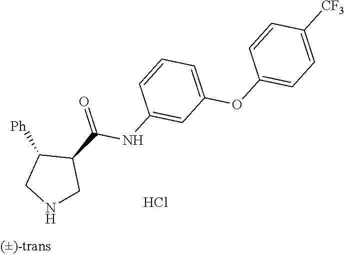 |
| I-93 | 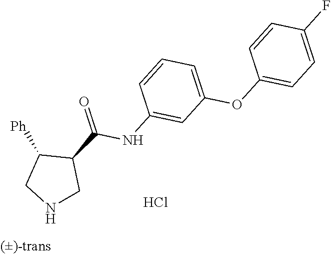 [SMILES: O=C(Nc1cccc(Oc2ccc(F)cc2)c1)[C@H]1CNC[C@@H]1c1ccccc1 ; Cl] |
| I-94 | 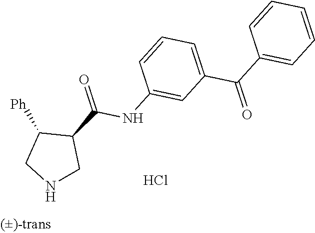 |
| I-95 | 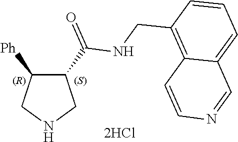 |
| I-96 | 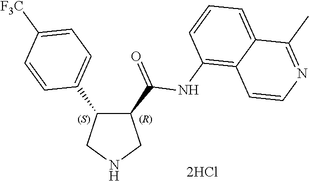 |
| I-97 | 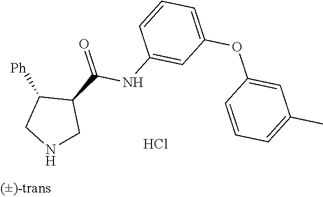 [SMILES: Cc1cccc(Oc2cccc(NC(=O)[C@H]3CNC[C@@H]3c3ccccc3)c2)c1 ; Cl] |
| I-98 | 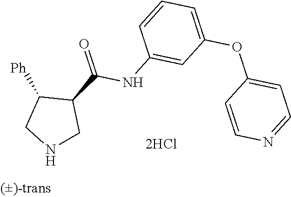 [SMILES: O=C(Nc1cccc(Oc2ccncc2)c1)[C@H]1CNC[C@@H]1c1ccccc1 ; "2HCl"] |
| I-99 | 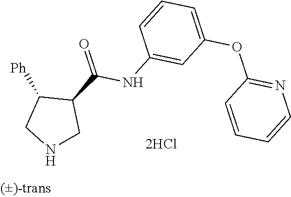 [SMILES: O=C(Nc1cccc(Oc2ccccn2)c1)[C@H]1CNC[C@@H]1c1ccccc1 ; "2HCl"] |
| I-100 | 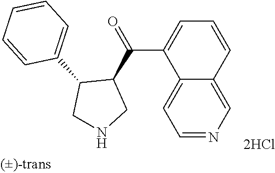 [SMILES: O=C(c1cccc2cnccc12)[C@H]1CNC[C@@H]1c1ccccc1] |
| I-101 | 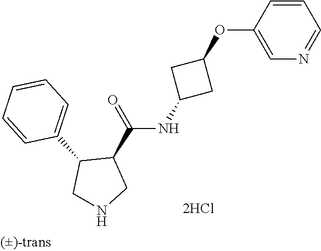 [SMILES: O=C(N[C@H]1C[C@H](Oc2cccnc2)C1)[C@H]1CNC[C@@H]1c1ccccc1] |
| I-102 | 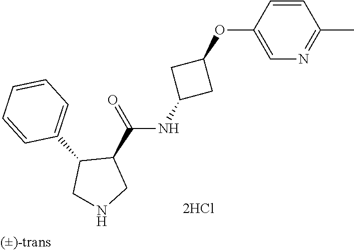 [SMILES: Cc1ccc(O[C@H]2C[C@H](NC(=O)[C@H]3CNC[C@@H]3c3ccccc3)C2)cn1] |
| I-103 | 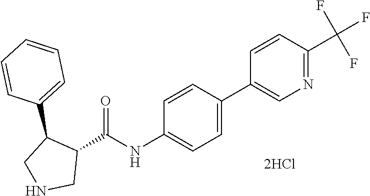 [SMILES: O=C(Nc1ccc(-c2ccc(C(F)(F)F)nc2)cc1)[C@@H]1CNC[C@H]1c1ccccc1] |
| I-104 | 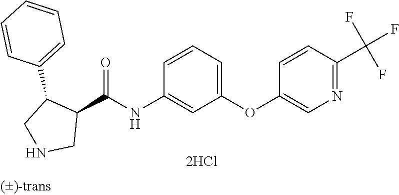 [SMILES: O=C(Nc1cccc(Oc2ccc(C(F)(F)F)nc2)c1)[C@H]1CNC[C@@H]1c1ccccc1] |
| I-105 | 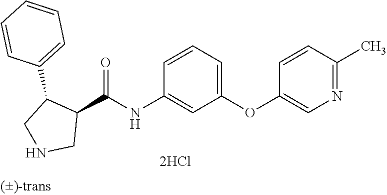 [SMILES: Cc1ccc(Oc2cccc(NC(=O)[C@H]3CNC[C@@H]3c3ccccc3)c2)cn1] |
| I-106 | 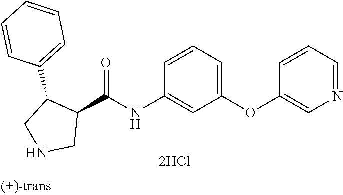 [SMILES: O=C(Nc1cccc(Oc2cccnc2)c1)[C@H]1CNC[C@@H]1c1ccccc1] |
| I-107 | 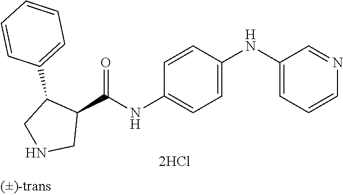 [SMILES: O=C(Nc1ccc(Nc2cccnc2)cc1)[C@H]1CNC[C@@H]1c1ccccc1] |
| I-108 | 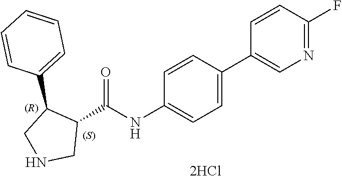 |
| I-109 | 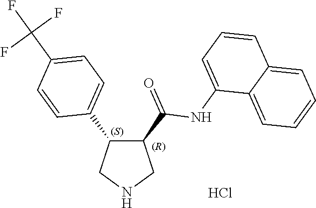 |
| I-110 | 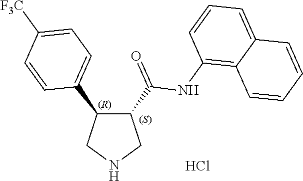 |
| I-111 | 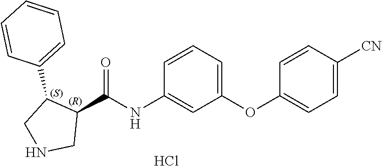 |
| I-112 | 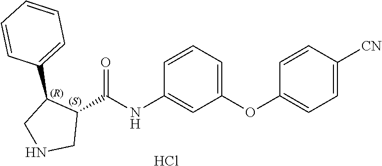 |
| I-113 | 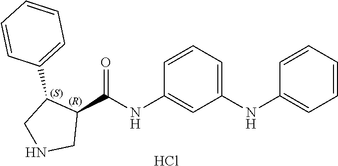 |
| I-114 | 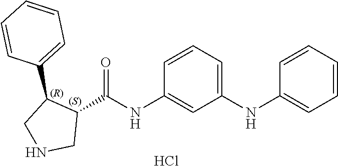 |
| I-115 | 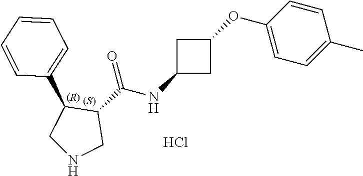 |
| I-116 | 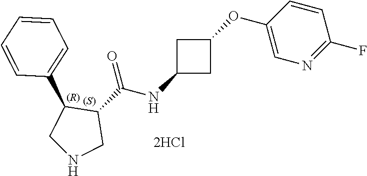 |
| I-117 | 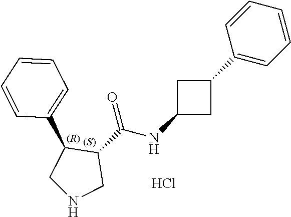 |
| I-118 | 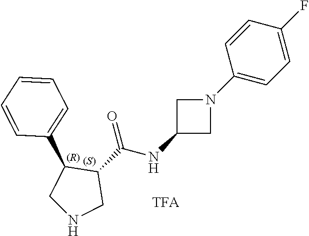 |
| I-119 | 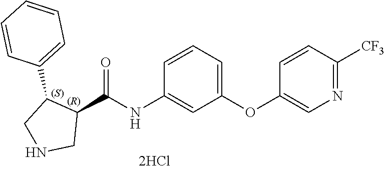 |
| I-120 | 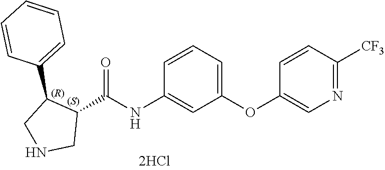 |
| I-121 | 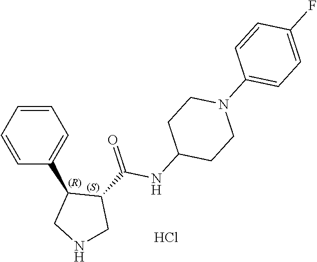 |
| I-122 | 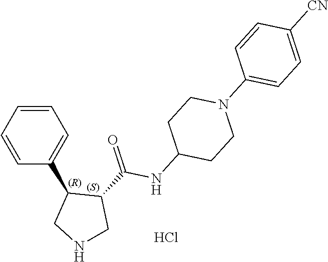 |
| I-123 | 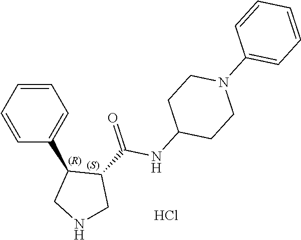 |

Embodiment II-74. A pharmaceutical composition, comprising the compound of any one of Embodiments II-1 to II-73, or a pharmaceutically acceptable salt or tautomer thereof, and a pharmaceutically acceptable excipient.

Embodiment II-75. A method of modulating activity of NR2F6 by exposure of NR2F6 to an effective amount of a compound of any one of Embodiments II-1 to II-73, or a pharmaceutically acceptable salt or tautomer thereof, or the pharmaceutical composition of Embodiment II-74.

Embodiment II-76. The method of Embodiment II-75, wherein said modulation comprises of augmentation of NR2F6 activity.

Embodiment II-77. The method of Embodiment II-75, wherein said modulation comprise of inhibition of NR2F6 activity.

Embodiment II-78. A method of treating or reducing the effect of a disease or disorder associated with NR2F6 modulation, the method comprising administration of an effective amount of a compound of any one of Embodiments II-1 to II-73, or a pharmaceutically acceptable salt or tautomer thereof, or the pharmaceutical composition of Embodiment II-76.

Embodiment II-79. The method of Embodiment II-78, wherein the disease or disorder comprises an augmented autoimmune response.

Embodiment II-80. The method according to Embodiment II-79, wherein the augmented autoimmune response is selected from the group consisting of rheumatoid arthritis, systemic lupus erythematosiss (lupus), inflammatory bowel disease, multiple sclerosis, type-1 diabetes mellitus, Guillian-Barre syndrome, chronic inflammatory demyelinating polyneuropathy, psoriasis/psoriatic arthritis, Grave's disease, Hashimoto's thyroiditis, myasthenia gravis, and vasculitis.

Embodiment II-81. The method of Embodiment II-78, wherein the disorder is cancer.

Embodiment II-82. The method according to Embodiment II-81, wherein the cancer is a solid tumor selected from the group consisting of adenocarcinoma of the lung, bile duct cancer, bladder cancer; bone cancer, brain tumor, glioma, anaplastic oligodendroglioma, adult glioblastoma multiforme, adult anaplastic astrocytoma; benign prostate hyperplasia bronchoalveolar carcinoma, breast cancer, including metastatic breast cancer; cervical cancer, cholangiocarcinoma, colorectal cancer, esophageal cancer, gastric cancer, head and neck cancer, squamous cell carcinoma of the head and neck, gallbladder cancer hepatocellular cancer, kidney cancer, liver cancer, lung cancer, melanoma; neuroendocrine cancer, metastatic neuroendocrine tumor, non-small cell lung cancer (NSCLC), small cell lung cancer, ovarian cancer, primary peritoneal cancer, pancreatic cancer, prostate cancer, including androgen-dependent and androgen-independent prostate cancer, colorectal carcinoma, renal cancer, metastatic renal cell carcinoma, soft tissue sarcoma, urinary bladder cancer, and uterine cancer.

Embodiment II-83. The method of Embodiment II-78, wherein the disorder is a haematological malignancy.

Embodiment II-84. The method of Embodiment II-83, wherein the hematologic malignancy is selected from the group consisting of acute myeloid leukemia, chronic myelogenous leukemia (CML), accelerated CML, CML blast phase (CML-BP), acute lymphoblastic leukemia, chronic lymphocytic leukemia (CLL), Hodgkin's disease, non-Hodgkin's lymphoma, follicular lymphoma, mantle cell lymphoma, B-cell lymphoma, T-cell lymphoma, multiple myeloma, Waldenstrom's macroglobulinemia, myelodysplastic syndromes (MDS), refractory anemia (RA), RA with ringed sideroblasts, RA with excess blasts (RAEB), RAEB in transformation, and a myeloproliferative syndrome.

Embodiment II-85. A method of treating or reducing the effect of a gastrointestinal disease or disorder, the method comprising administration of an effective amount of a compound of any one of Embodiments II-1 to II-73, or a pharmaceutically acceptable salt or tautomer thereof, or the pharmaceutical composition of Embodiment II-4.

Embodiment II-86. The method of Embodiment II-85, wherein the gastrointestinal disorder is IBD, Crohn's disease, or colitis.

Embodiment II-87. A method of treating a condition associated with hepatic steatosis, the method comprising administration of an effective amount of a compound of any one of Embodiments II-1 to II-73, or a pharmaceutically acceptable salt or tautomer thereof, or the pharmaceutical composition of Embodiment II-74.

Embodiment II-88. The method of Embodiment II-87, wherein the condition associated with hepatic steatosis is non-alcoholic fatty liver disease (NAFLD) or non-alcoholic steatohepatitis (NASH).

Embodiment II-89. A compound of any one of Embodiments II-1 to II-73, or a pharmaceutically acceptable salt or tautomer thereof, or the pharmaceutical composition of Embodiment II-74 for use in modulating activity of NR2F6.

Embodiment II-90. A compound of any one of Embodiments II-1 to II-73, or a pharmaceutically acceptable salt or tautomer thereof, or the pharmaceutical composition of Embodiment II-74 for use in treating or reducing the effect of a disease or disorder associated with NR2F6 modulation.

Embodiment II-91. Use of a compound of any one of Embodiments II-1 to II-73, or a pharmaceutically acceptable salt or tautomer thereof, or the pharmaceutical composition of Embodiment II-74, for modulating activity of NR2F6.

Embodiment II-92. Use of a compound of any one of Embodiments II-1 to II-73, or a pharmaceutically acceptable salt or tautomer thereof, or the pharmaceutical composition of Embodiment II-74, for treating or reducing the effect of a disease or disorder associated with NR2F6 modulation.

Embodiment II-93. Use of a compound of any one of v, or a pharmaceutically acceptable salt or tautomer thereof, or the pharmaceutical composition of Embodiment II-74, in the manufacture of a medicament for modulating activity of NR2F6.

Embodiment II-94. Use of a compound of any one of Embodiments II-1 to II-73, or a pharmaceutically acceptable salt or tautomer thereof, or the pharmaceutical composition of Embodiment II-4, in the manufacture of a medicament for treating or reducing the effect of a disease or disorder associated with NR2F6 modulation.

### EXAMPLES

All percentages and ratios used herein, unless otherwise indicated, are by weight. Other features and advantages of the present disclosure will become apparent from the different examples. The provided examples illustrate different components and methodology useful in practicing the present disclosure. Generally speaking, the disclosure extends to any novel one, or any novel combination, of the features disclosed in this specification (including the accompanying claims and drawings). The examples do not limit the claimed disclosure. Thus, features, integers, characteristics, compounds or chemical moieties described in conjunction with a particular aspect, embodiment or example of the disclosure are to be understood to be applicable to any other aspect, embodiment or example described herein, unless incompatible therewith. Based on the present disclosure the skilled artisan can identify and employ other components and methodology useful for practicing the present disclosure. Moreover, unless stated otherwise, any feature disclosed herein may be replaced by an alternative feature serving the same or a similar purpose.

The Disclosure will now be described by way of example only with reference to the Examples below:

### EXEMPLIFICATION

### Compound Preparation

### General Methods and Materials

All chemicals were purchased from Sigma-Aldrich, Alfa Aesar. ¹H NMR spectra were recorded at 200 and 400 MHz and ¹³C NMR spectra were recorded at 100.6 and 50.3 MHz by using deuterated solvents indicated below. TLCs were performed on aluminum backed silica plates (silica gel 60 F254). All the reactions were performed under nitrogen atmosphere using distilled solvents. All tested compounds were found to have >95% purity determined by HPLC analysis. HPLC-grade water was obtained from a tandem Milli-Ro/Milli-Q apparatus. The analytical HPLC measurements were made on a Shimadzu LC-20AProminence equipped with a CBM-20A communication bus module, two LC-20AD dual piston pumps, a SPD-M20A photodiode array detector and a Rheodyne 7725i injector with a 20 μL stainless steel loop.

Abbreviations used in the following examples and elsewhere herein are:
Ac₂O acetic anhydride
AcOH acetic acid
AIBN Azobisisobutyronitrile
atm atmosphere
brs broad singlet
DIPEA N,N-diisopropyl ethyl amine
DCM dichloromethane
DME dimethoxy ethane
DMF N,N-dimethylformamide
DMSO dimethyl sulfoxide
d doublet
dd doublets of doublet
EDC N-(3-Dimethylaminopropyl)-M-ethylcarbodiimide hydrochloride
ESI electrospray ionization
EtMgBr ethyl magnesium bromide
EtOAc ethyl acetate
Et₂O diethyl ether
EtOH ethanol
EtO⁻Na⁺ sodium ethoxide
h hour(s)
HATU 1-[Bis(dimethylamino)methylene]-1H-1,2,3-triazolo[4,5-b]pyridinium 3-oxide hexafluorophosphate
HPLC high-performance liquid chromatography
iPrOH iso-propanol
LCMS liquid chromatography-mass spectrometry
m multiplet
MeI methyl iodide
MeOH methanol
MHz megahertz
min minute(s)
MS molecular sieves
MW microwave
NBS N-bromosuccinamide
NMR nuclear magnetic resonance
PET petroleum ether
ppm parts per million
p-TSA para-toluenesulfonic acid
q quartet
r.t. room temperature
s singlet
TLC thin layer chromatography
THF tetrahydrofuran
t triplet
UHPLC ultra high-performance liquid chromatography
v/v volume-to-volume

### Example 1: 1-(tert-Butoxycarbonyl)-4-phenyl-2,5-dihydro-1H-pyrrole-3-carboxylic acid (1.6)

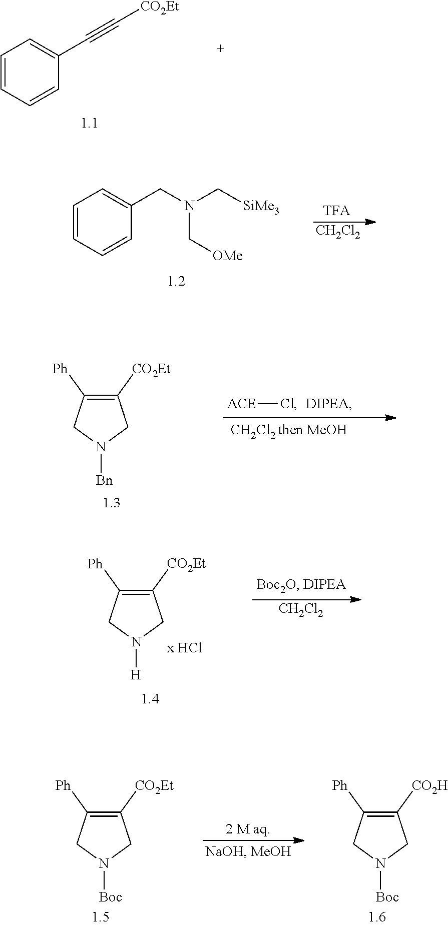

### Step 1: Ethyl 1-benzyl-4-phenyl-2,5-dihydro-1H-pyrrole-3-carboxylate (1.3)

A solution of TFA (0.15 mL, 1.98 mmol) in CH₂Cl₂ (3 mL), was added dropwise to a stirred solution of intermediate 1.1 (2.0 g, 11.48 mmol) and intermediate 1.2 (8.1 mL, 31.69 mmol) in CH₂Cl₂ (50 mL) cooled at 0-5° C. The resulting mixture was stirred at r.t. for 18 h. The reaction was poured into H₂O (100 mL), the two phases were separated, and the organic phase was washed with brine (100 mL), aq. NaHCO₃ ss (100 mL), dried over Na₂SO₄, and concentrated under reduced pressure. The crude was purified by flash chromatography (PET/EtOAc from 100% PET to 80:20 v/v PET/EtOAc). The intermediate 1.3 (2.71 g, 8.82 mmol) was obtained in 77% yield. MS-ESI(+) m/z: 308.4 (M+H).

### Step 2: 1-tert-Butyl 3-ethyl 4-phenyl-2,5-dihydro-1H-pyrrole-1,3-dicarboxylate (1.5)

DIPEA (1.75 mL, 10.03 mmol), and 1-chloroethylchloroformate (2.45 mL, 22.79 mmol) were added to a stirred solution of intermediate 1.3 (2.80 g, 9.12 mmol) in CH₂Cl₂ (100 mL), and the resulting mixture was stirred at reflux for 1 h. Once cooled at room temperature volatiles were removed under reduced pressure. The crude was dissolved in MeOH (50 mL) and vigorously stirred under reflux for 1 h. The reaction was cooled at room temperature and concentrated under reduced pressure. The obtained oil residue (intermediate 1.4) was dissolved in CH₂Cl₂ (70 mL) and reacted with Boc₂O (2.38 g, 10.94 mmol) and DIPEA (4.77 mL, 27.35 mmol) at r.t. for 3 h. The mixture was washed with 0.5 M aq. citric acid (50 mL), 10% aq. NaHCO₃ (50 mL), brine (50 mL), dried over Na₂SO₄, and concentrated under reduced pressure. 4.2 g of the intermediate 1.5 were obtained and used such as for the next step. MS-ESI(+) m/z 318.3 (M+H-100).

### Step 3: 1-(tert-Butoxycarbonyl)-4-phenyl-2,5-dihydro-1H-pyrrole-3-carboxylic acid (1.6)

A stirred solution of intermediate 1.5 (crude of previous step, 9.12 mmol) in MeOH (45 mL) was treated with 2.0 M aq. NaOH (45.5 mL, 91.15 mmol) at r.t. for 1 h. The mixture was then concentrated under reduced pressure to ⅓ of the initial volume and diluted with 50 mL of H₂O. The solution was washed with Et₂O (3×25 mL) and then acidified to pH=1 by adding 37% HCl. The aqueous phase was extracted with EtOAc (3×50 mL), washed with brine (50 mL), dried over Na₂SO₄, and concentrated under reduced pressure. 1.77 g of the title intermediate 1.6 were obtained as a pale brown solid (yield: 67% from intermediate 1.3). MS-ESI(−) m/z 288.1 (M−H).

### Example 2: tert-Butyl (±)-trans-3-hydroxy-4-phenylpyrrolidine-1-carboxylate (2.2)

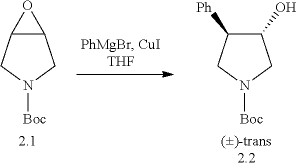 [SMILES: CC(C)(C)OC(=O)N1CC2OC2C1 ; CC(C)(C)OC(=O)N1C[C@@H](O)[C@H](c2ccccc2)C1]

### Step 1: tert-Butyl (±)-trans-3-hydroxy-4-phenylpyrrolidine-1-carboxylate (2.2)

A solution of intermediate 2.1 (2.36 g, 12.74 mmol) in THF (20 mL) was added dropwise to a stirred solution of 3.0 M phenylmagnesium bromide in Et₂O (8.5 mL, 25.48 mmol) and CuI (0.12 g, 0.63 mmol) in THF (20 mL) cooled at 0-5° C. The reaction was slowly warmed to r.t. and stirred for 3 h. The mixture was then diluted with EtOAc (50 mL) and cautiously quenched by adding brine (50 mL). The two phases were separated and the aqueous phase was extracted with EtOAc (2×50 mL). The collected organic layers were washed with 0.5 M aq. citric acid (30 mL), and brine (30 mL), dried over Na₂SO₄, and concentrated under reduced pressure. The crude was purified by flash chromatography (PET/EtOAc from 85:15 to 40:60 v/v), to give 3.17 g (23.92 mmol) of the title intermediate 2.2 (94%). MS-ESI(−) m/z 262.6 (M−H).

### Example 3: tert-Butyl (±)-cis-3-hydroxy-4-phenylpyrrolidine-1-carboxylate (3.3)

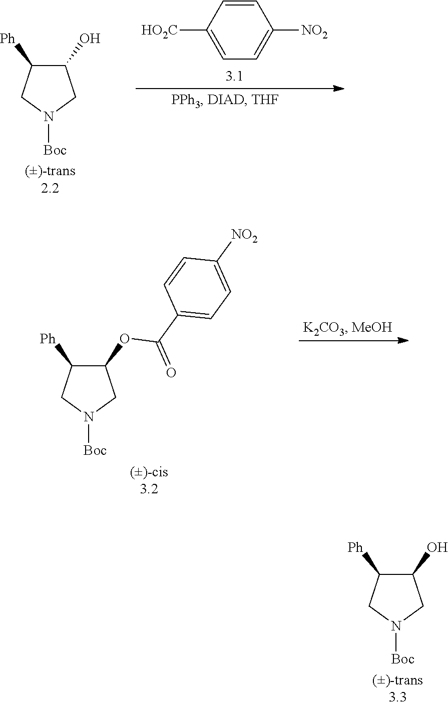 [SMILES: CC(C)(C)OC(=O)N1C[C@@H](O)[C@H](c2ccccc2)C1 ; O=C(O)c1ccc([N+](=O)[O-])cc1 ; CC(C)(C)OC(=O)N1C[C@H](OC(=O)c2ccc([N+](=O)[O-])cc2)[C@H](c2ccccc2)C1 ; CC(C)(C)OC(=O)N1C[C@H](O)[C@H](c2ccccc2)C1]

### Step 1: tert-Butyl (±)-cis 3-phenyl-4-{[(4-nitrophenyl)carbonyl]oxy}pyrrolidine-1-carboxylate (3.2)

A solution of intermediate 2.2 (1.06 g, 4.01 mmol) and triphenylphosphine (1.26 g, 4.81 mmol) in THF (10 mL) was added dropwise to a stirred solution of DIAD (0.94 mL, 4.812 mmol) and intermediate 3.1 (0.80 g, 4.812 mmol) in THF (20 mL) under a N₂ atmosphere and cooled at 0-5° C. The mixture was stirred at r.t. for 16 h, and then poured into aq. NaHCO₃ ss (20 mL). The two phases were separated and the aqueous phase was extracted with EtOAc (2×50 mL). The collected organic layers were washed with 0.5 M aq. citric acid (30 mL), brine (30 mL), dried over Na₂SO₄, and concentrated under reduced pressure. The crude was purified by flash chromatography (PET/EtOAc from 90:10 to 70:30 v/v), to give 1.64 g (3.98 mmol) of the intermediate 3.2 (99%). MS-ESI(−) m/z 411.5 (M−H).

### Step 2: tert-Butyl (±)-cis-3-hydroxy-4-phenylpyrrolidine-1-carboxylate (3.3)

A stirred solution of intermediate 3.2 (1.64 g, 3.98 mmol) in MeOH (15 mL) was treated with K₂CO₃ (2.19 g, 15.91 mmol) at r.t. for 1 h. The mixture was diluted with EtOAc (50 mL), washed with H₂O (30 mL), brine (30 mL), dried over Na₂SO₄, and concentrated under reduced pressure. The crude was purified by flash chromatography (PET/EtOAc from 90:10 to 60:40 v/v), to give 0.95 g (3.60 mmol) of the title intermediate 3.3 (89%). MS-ESI(−) m/z 262.6 (M−H).

### Example 4: (±)-trans-1-Benzyl-4-phenylpyrrolidin-3-amine (4.3)

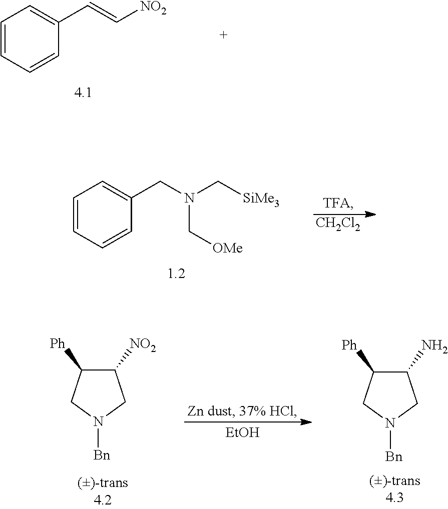 [SMILES: "EtOH" ; O=[N+]([O-])/C=C/c1ccccc1 ; COCN(Cc1ccccc1)C[Si](C)(C)C ; "CH2Cl2" ; O=[N+]([O-])[C@@H]1CN(Cc2ccccc2)C[C@H]1c1ccccc1 ; N[C@@H]1CN(Cc2ccccc2)C[C@H]1c1ccccc1]

### Step 1: (±)-trans-1-Benzyl-3-nitro-4-phenylpyrrolidine (4.2)

Intermediate 4.2 was synthesized according to the procedure described in Step 1 of Example 1 from intermediate 4.1 (2.00 g, 13.41 mmol), intermediate 1.2 (4.11 mL, 16.09 mmol), and TFA (0.10 mL, 1.34 mmol) in CH₂Cl₂ (20 mL). The intermediate 4.2 (2.44 g, 8.64 mmol) was obtained after work-up and chromatographic purification (PET/EtOAc, from 8:2 v/v to 2:8 v/v). Yield: 64%. MS-ESI(+) m/z: 283.3 (M+H).

### Step 2: (H-trans-1-Benzyl-4-phenylpyrrolidin-3-amine (4.3)

37% HCl (2.6 mL, 31.20 mmol) was added to a solution of intermediate 4.2 (400 mg, 1.42 mmol) in EtOH (5 mL), zinc dust (741 mg, 11.34 mmol) was then cautiously added portion wise, and the resulting mixture was stirred at r.t. for 16 h. The mixture was then poured into 28% aq. NH₃ (20 mL) and extracted with CH₂Cl₂ (3×20 mL). The collected organic layers were washed with brine (20 mL), dried over Na₂SO₄, and concentrated under reduced pressure, to give 294 mg (1.17 mmol) of the title intermediate 4.3 in 82% yield. MS-ESI(+) m/z: 253.1 (M+H).

### Example 5: tert-Butyl (±)-trans-3-amino-4-phenylpyrrolidine-1-carboxylate (5.3)

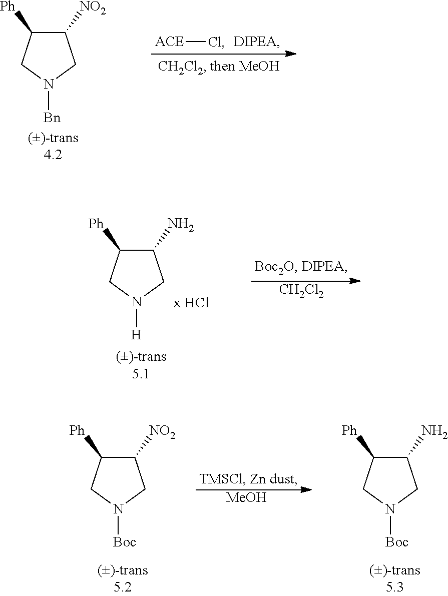

### Step 1: tert-Butyl (±)-trans-3-nitro-4-phenylpyrrolidine-1-carboxylate (5.2)

Intermediate 5.1 was synthesized according to the procedure described in Step 2 of Example 1 from intermediate 4.2 (710 mg, 2.52 mmol), DIPEA (0.48 mL, 2.77 mmol), and 1-chloroethylchloroformate (0.68 mL, 6.29 mmol) in CH₂Cl₂ (30 mL). The obtained crude was treated in refluxing MeOH (10 mL). After removal of volatiles, the intermediate 5.1 was reacted with Boc₂O (0.66 g, 3.02 mmol) and DIPEA (1.31 mL, 7.55 mmol) in CH₂Cl₂ (30 mL). After work-up and chromatographic purification (PET/EtOAc, from 80:20 to 50:50, v/v), the intermediate 5.2 was obtained in 79% yield (582 mg, 1.99 mmol). MS-ESI(+) m/z 293.1 (M+H).

### Step 2: tert-Butyl (±)-trans-3-amino-4-phenylpyrrolidine-1-carboxylate (5.3)

TMSCl (5.15 mL, 40.63 mmol) and Zn dust (2.81 g, 43.02 mmol) were added sequentially to a stirred solution of intermediate 5.2 (585 mg, 2.00 mmol) in MeOH (10 mL) cooled at 0° C., and the resulting mixture was reacted at the same conditions for 1 h. The mixture was filtered through a celite pad under vacuum. The collected liquor was diluted with CH₂Cl₂ (50 mL), washed with aq. NaHCO₃ ss (30 mL), brine (30 mL), dried over Na₂SO₄, and concentrated under reduced pressure, to give 400 mg of title intermediate 5.3 which was used such as without purification. MS-ESI(+) m/z 263.4 (M+H).

### Example 6: (±)-trans-1-(tert-Butoxycarbonyl)-4-phenylpyrrolidine-3-carboxylic acid (6.5)

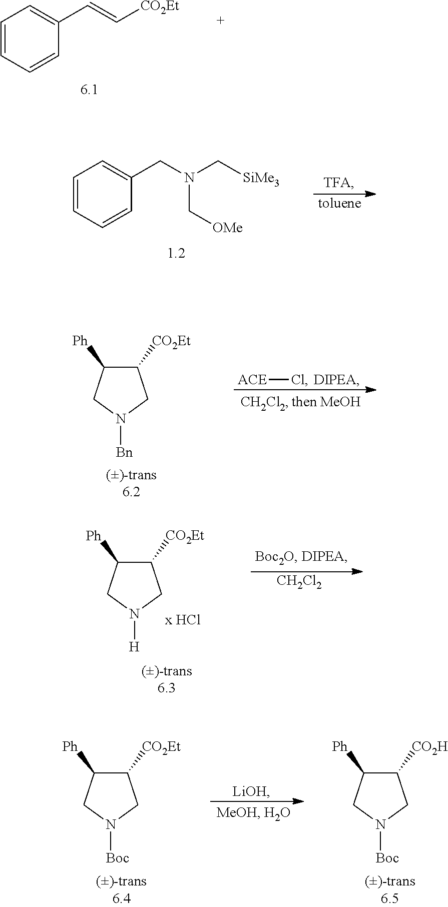

### Step 1: Ethyl (±)-trans I-benzyl-4-phenylpyrrolidine-3-carboxylate (6.2)

A solution of TFA (0.95 mL, 12.50 mmol) in toluene (10 mL) was added dropwise to a stirred solution of intermediate 6.1 (7.00 mL, 41.67 mmol) and intermediate 1.2 (11.7 mL, 45.84 mmol) in toluene (50 mL) cooled at 0-5° C. The resulting mixture was stirred at r.t. for 48 h. The reaction was poured into EtOAc (50 mL) and FLO (50 mL), the two phases were separated, and the organic phase was washed with aq. NaHCO₃ ss (60 mL), brine (60 mL), dried over Na₂SO₄, and concentrated under reduced pressure. The crude was purified by flash chromatography (PET/EtOAc, from 90:10 to 70:30 v/v PET/EtOAc). 7.01 g of intermediate 6.2 were obtained as a colorless oil (yield: 54%). MS-ESI(+) m/z: 310.5 (M+H).

### Step 2: 1-tert-Butyl 3-ethyl (H-trans-4-phenylpyrrolidine-1,3-dicarboxylate (6.4)

Intermediate 6.3 was synthesized according to the procedure described in Step 2 of Example 1 from intermediate 6.2 (7.00 g, 22.62 mmol), DIPEA (4.33 mL, 24.89 mmol), and 1-chloroethylchloroformate (6.17 mL, 57.24 mmol) in CH₂Cl₂ (100 mL). The obtained crude was treated in refluxing MeOH (100 mL). After removal of volatiles, the intermediate 6.3 was reacted with Boc₂O (5.43 g, 24.89 mmol) and DIPEA (11.82 mL, 7.55 mmol) in CH₂Cl₂ (100 mL). After work-up, the crude of intermediate 6.4 was used such without purification. MS-ESI(+) m/z 320.4 (M+H).

### Step 3: (±)-trans-1-(tert-Butoxycarbonyl)-4-phenylpyrrolidine-3-carboxylic acid (6.5)

Aq. LiOH 4.0 M (28 mL, 0.11 mol) was added to a stirred solution of intermediate 6.4 (crude of previous step, 22.62 mmol) in MeOH (75 mL) and H₂O (15 mL), and the reaction was vigorously stirred at r.t. for 4 h. Then, the mixture was concentrated under reduced pressure up to ¼ of initial volume, H₂O (30 mL) was added and the opalescent solution was washed with Et₂O (3×50 mL). The aqueous phase was acidified up to pH=1 by adding 37% HCl, and the obtained suspension was extracted with CH₂Cl₂ (3×50 mL). The collected organic layers were washed with brine (2×50 mL), dried over Na₂SO₄, and concentrated under reduced pressure. 5.95 g of the title intermediate 6.5 were obtained as a white powder (90% yield from intermediate 9.2). MS-ESI(−) m/z 290.1 (M−H).

### Example 7: (±)-trans-1-(tert-Butoxycarbonyl)-4-(thiophen-2-yl)pyrrolidine-3-carboxylic acid (7.7)

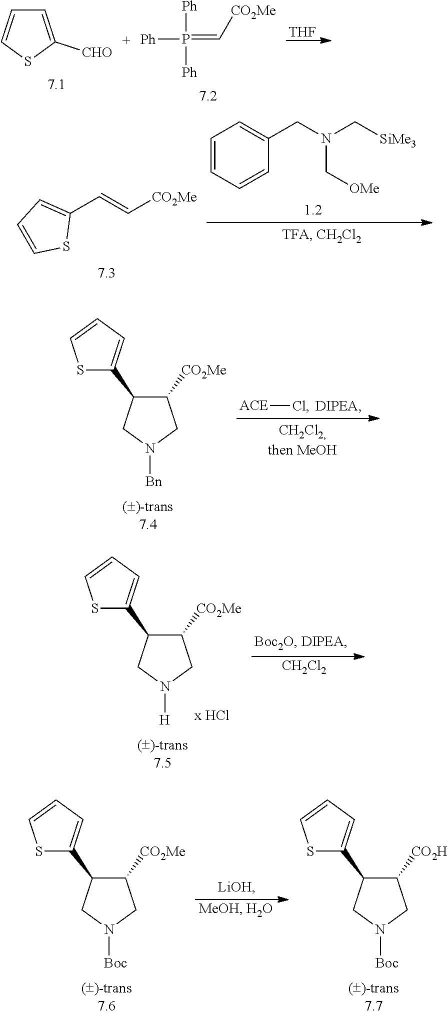

### Step 1: Methyl (2E)-3-(thiophen-2-yl)prop-2-enoate (7.3)

Intermediate 7.2 (1.49 g, 4.46 mmol) was added to a stirred solution of intermediate 7.1 (0.33 mL, 3.57 mmol) in THF (10 mL) under N2 atmosphere, and the resulting mixture was stirred at r.t. for 24 h. The mixture was concentrated under reduced pressure and purified by flash chromatography (PET/EtOAc from 95:5 to 80:20 v/v PET/EtOAc), to give 561 mg (3.34 mmol) of intermediate 7.3 (93%) as white crystals. MS-ESI(−) m/z: 167.4 (M−H).

### Step 2: Methyl (±)-trans-1-benzyl-4-(thiophen-2-yl)pyrrolidine-3-carboxylate (7.4)

Intermediate 7.4 was synthesized according to the procedure described in Step 1 of Example 1 from intermediate 7.3 (548 mg, 3.26 mmol), 1.2 (1.08 mL, 4.24 mmol) and TFA (0.025 mL, 0.33 mmol) in CH₂Cl₂ (7.0 mL). The intermediate 7.4 (740 mg, 2.46 mmol) was obtained after work-up and chromatographic purification (PET/EtOAc, from 100% PET to PET/EtOAc 8:2 v/v). Yield: 75%. MS-ESI(+) m/z: 302.5 (M+H).

### Step 3: 1-tert-Butyl 3-methyl (±)-trans-4-(thiophen-2-yl)pyrrolidine-1,3-dicarboxylate (7.6)

Intermediate 7.6 was synthesized according to the procedure described in Step 2 of Example 1 from intermediate 7.4 (580 mg, 1.92 mmol), DIPEA (0.37 mL, 2.12 mmol) and 1-chloroethylchloroformate (0.52 mL, 4.81 mmol) in CH₂Cl₂ (15 mL). The crude obtained was treated in refluxing MeOH (10 mL). After removal of volatiles, the intermediate 7.5 was reacted with Boc₂O (630 mg, 2.87 mmol) and DIPEA (1.00 mL, 5.77 mmol) in CH₂Cl₂ (20 mL). After work-up and chromatographic purification (PET/EtOAc, from 90:10 to 70:30, v/v), the intermediate 7.6 was obtained in 70% yield. MS-ESI(+) m/z 312.6 (M+H).

### Step 4: (±)-trans-1-(tert-Butoxycarbonyl)-4-(thiophen-2-yl)pyrrolidine-3-carboxylic acid (7.7)

Intermediate 7.7 was synthesized according to the procedure described in Step 3 of Example 6 from intermediate 7.6 (460 mg, 1.48 mmol), 4.0 M aq. LiOH (1.84 mL, 7.39 mmol) in MeOH (4 mL) and H₂O (1 mL). After workup, the title intermediate 7.7 was obtained as a white solid (440 mg, 1.48 mmol). Yield: quantitative. MS-ESI(−) m/z: 296.6 (M−H).

### Example 8: (±)-trans-1-(tert-Butoxycarbonyl)-4-(4-fluorophenyl)pyrrolidine-3-carboxylic acid (8.6)

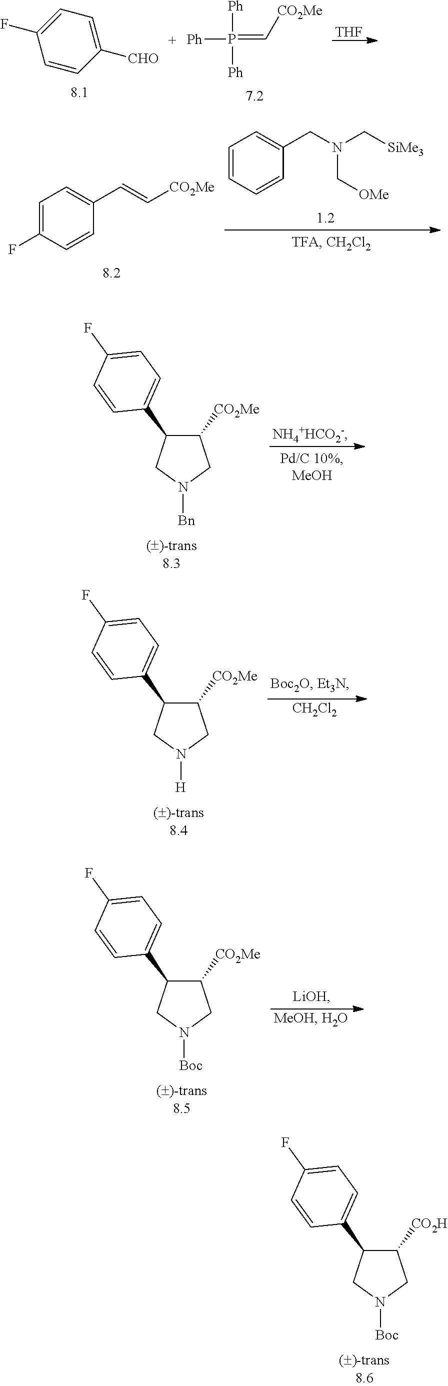

### Step 1: Methyl (2E)-3-(4-fluorophenyl)prop-2-enoate (8.2)

Intermediate 8.2 was synthesized according to the procedure described in Step 1 of Example 7 from intermediate 8.1 (0.34 mL, 3.22 mmol) and intermediate 7.2 (1.35 g, 4.03 mmol) in THF (10 mL). The intermediate 8.2 (557 mg) was obtained as white crystals after chromatographic purification (PET/EtOAc from 95:5 to 80:20, v/v). Yield: 96%. MS-ESI(−) m/z: 179.1 (M−H).

### Step 2: Methyl (±)-trans-1-benzyl-4-(4-fluorophenyl)pyrrolidine-3-carboxylate (8.3)

Intermediate 8.3 was synthesized according to the procedure described in Step 1 of Example 1 from intermediate 8.2 (544 mg, 3.02 mmol), intermediate 1.2 (1.0 mL, 3.92 mmol), and TFA (0.023 mL, 0.30 mmol) in CH₂Cl₂ (6.5 mL). The intermediate 8.3 (689 mg, 2.20 mmol) was obtained after work-up and chromatographic purification (PET/EtOAc, from 100% PET to PET/EtOAc 8:2 v/v). Yield: 73%. MS-ESI(+) m/z: 314.5 (M+H).

### Step 3: 1-tert-Butyl 3-methyl (±)-trans-4-(4-fluorophenyl)pyrrolidine-1,3-dicarboxylate (8.5)

Ammonium formate (410 mg, 6.51 mmol) and 10% Pd/C (68 mg) were added to a stirred solution of 8.3 (680 mg, 2.17 mmol) in MeOH (5 mL) under N2 atmosphere, and the resulting mixture was stirred at 70° C. for 1 h. Once cooled to r.t., the mixture was filtered under vacuum through a celite pad, to give a methanolic solution of 8.4. This solution was cooled to 0° C. and Et₃N (1.51 mL, 10.85 mmol) and Boc₂O (1.42 g, 6.51 mmol) were added. The resulting mixture was reacted at r.t. for 3 h. Volatiles were removed under reduced pressure, the crude was poured into EtOAc (15 mL) and washed with 0.5 M aq. citric acid (15 mL), and brine (15 mL). The organic phase was dried over Na₂SO₄ and concentrated under reduced pressure. After chromatographic purification (PET/EtOAc from 90:10 to 70:30 v/v), the intermediate 8.5 was obtained as a colorless oil (572 mg, 1.77 mmol, 81% yield). MS-ESI(+) m/z: 324.6 (M+H).

### Step 4: (±)-trans-1-(tert-Butoxycarbonyl)-4-(4-fluorophenyl)pyrrolidine-3-carboxylic acid (8.6)

Intermediate 8.6 was synthesized according to the procedure described in Step 3 of Example 6 from intermediate 8.5 (561 mg, 1.73 mmol), 4.0 M aq. LiOH (2.5 mL, 8.67 mmol) in MeOH (5 mL) and H₂O (1 mL). After workup, the title intermediate 8.6 was obtained as a white solid (417 mg, 1.35 mmol). Yield: 78%. MS-ESI(−) m/z: 308.5 (M−H).

### Example 9: (±)-trans-1-(tert-Butoxycarbonyl)-4-(3-fluorophenyl)pyrrolidine-3-carboxylic acid (12.7)

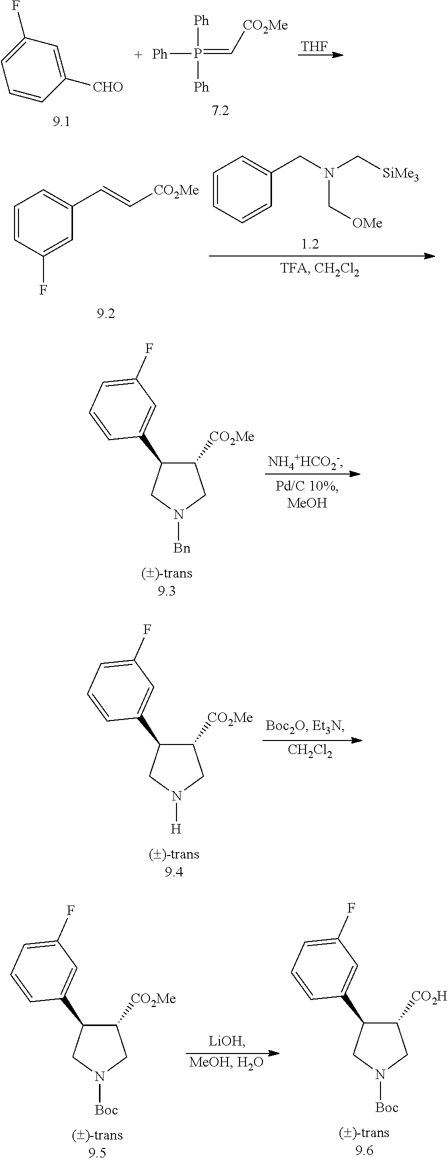

### Step 1: Methyl (2E)-3-(3-fluorophenyl)prop-2-enoate (9.2)

Intermediate 9.2 was synthesized according to the procedure described in Step 1 of Example 7 from 9.1 (0.34 mL, 3.22 mmol) and intermediate 7.2 (1.35 g, 4.03 mmol) in THF (10 mL). The intermediate 9.2 (550 mg, 3.05 mmol) was obtained as white crystals after chromatographic purification (PET/EtOAc, from 95:5 to 70:30, v/v). Yield: 95%. MS-ESI(−) m/z: 179.2 (M−H).

### Step 2: Methyl (±)-trans-1-benzyl-4-(3-fluorophenyl)pyrrolidine-3-carboxylate (9.3)

Intermediate 9.3 was synthesized according to the procedure described in Step 1 of Example 1 from intermediate 9.2 (537 mg, 2.98 mmol), intermediate 1.2 (0.99 mL, 3.87 mmol), and TFA (0.023 mL, 0.30 mmol) in CH₂Cl₂ (6.5 mL). The intermediate 9.3 (768 mg, 2.45 mmol) was obtained after work-up and chromatographic purification (PET/EtOAc, from 100% PET to PET/EtOAc 8:2 v/v). Yield: 82%. MS-ESI(−) m/z: 314.5 (M−H).

### Step 3: 1-tert-Butyl 3-methyl (±)-trans-4-(3-fluorophenyl)pyrrolidine-1,3-dicarboxylate (9.5)

Intermediate 9.4 was synthesized according to the procedure described in Step 3 of Example 8 from intermediate 9.3 (745 mg, 2.38 mmol), Pd/C 10% (70 mg), ammonium formate (450 mg, 7.13 mmol) in MeOH (10 mL). After filtration, the liquor containing the intermediate 9.4 was treated with Et₃N (1.65 mL, 11.89 mmol) and Boc₂O (1.55 g, 7.13 mmol). The title intermediate 9.5 (714 mg, 2.21 mmol) was obtained after work-up and chromatographic purification (PET/EtOAc, from 90:10 to 70:30, v/v). Yield: 93%. MS-ESI(+) m/z: 324.6 (M+H).

### Step 4: (±)-trans-1-(tert-Butoxycarbonyl)-4-(3-fluorophenyl)pyrrolidine-3-carboxylic acid (9.5)

Intermediate 9.6 was synthesized according to the procedure described in Step 3 of Example 6 from intermediate 9.5 (696 mg, 2.15 mmol), 4.0 M aq. LiOH (3.0 mL, 10.76 mmol) in MeOH (6 mL) and H₂O (1.5 mL). After workup, the title intermediate 9.6 was obtained as a white solid (573 mg, 1.85 mmol). Yield: 86%. MS-ESI(−) m/z: 308.5 (M−H).

### Example 10: (±)-trans-1-(tert-Butoxycarbonyl)-4-(2-fluorophenyl)pyrrolidine-3-carboxylic acid (10.6)

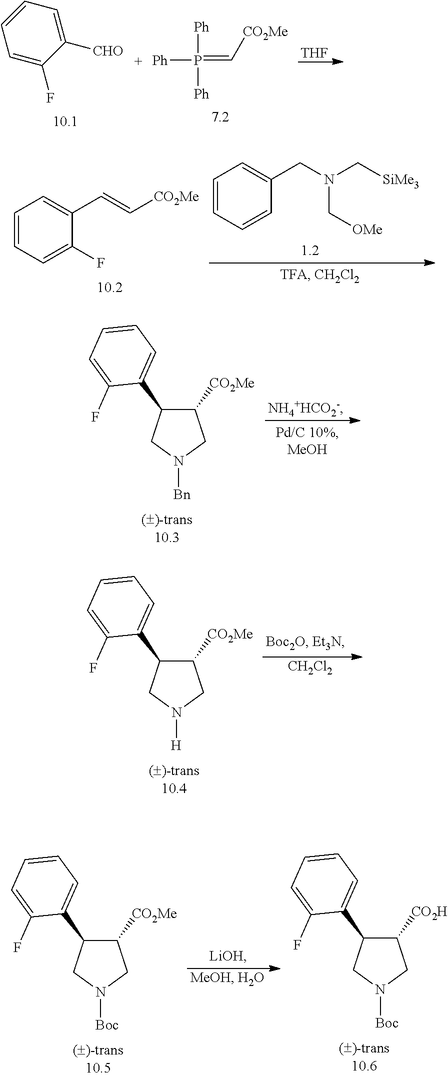

### Step 1: Methyl (2E)-3-(2-fluorophenyl)prop-2-enoate (10.2)

Intermediate 10.2 was synthesized according to the procedure described in Step 1 of Example 7 from intermediate 10.1 (0.25 mL, 2.42 mmol) and intermediate 7.2 (1.01 g, 3.02 mmol) in THF (8 mL). The intermediate 10.2 (408 mg, 2.26 mmol) was obtained as a colorless oil after chromatographic purification (PET/EtOAc, from 90:10 to 80:20, v/v). Yield: 94%. MS-ESI(−) m/z: 179.2 (M−H).

### Step 2: Methyl (±)-trans-1-benzyl-4-(2-fluorophenyl)pyrrolidine-3-carboxylate (10.3)

Intermediate 10.3 was synthesized according to the procedure described in Step 1 of Example 1 from intermediate 10.2 (394 mg, 2.19 mmol), intermediate 1.2 (0.73 mL, 2.84 mmol) and TFA (0.017 mL, 0.22 mmol) in CH₂Cl₂ (4.5 mL). The intermediate 10.3 (491 mg, 1.57 mmol) was obtained after work-up and chromatographic purification (PET/EtOAc, from 100% PET to PET/EtOAc 8:2 v/v). Yield: 72%. MS-ESI(−) m/z: 314.5 (M−H).

### Step 3: 1-tert-Butyl 3-methyl (±)-trans-4-(2-fluorophenyl)pyrrolidine-1,3-dicarboxylate (10.5)

Intermediate 10.5 was synthesized according to the procedure described in Step 3 of Example 8 from intermediate 10.3 (495 mg, 1.58 mmol), Pd/C 10% (50 mg), ammonium formate (299 mg, 4.74 mmol) in MeOH (10 mL). After filtration, the liquor containing the intermediate 10.4 was treated with Et₃N (1.10 mL, 7.90 mmol) and Boc₂O (1.03 g, 4.74 mmol). The intermediate 10.5 (475 mg, 1.47 mmol) was obtained after work-up and chromatographic purification (PET/EtOAc, from 90:10 to 70:30, v/v). Yield: 93%. MS-ESI(+) m/z: 324.5 (M+H).

### Step 4: (±)-trans-1-(tert-Butoxycarbonyl)-4-(2-fluorophenyl)pyrrolidine-3-carboxylic acid (10.6)

Intermediate 10.6 was synthesized according to the procedure described in Step 3 of Example 6 from intermediate 10.5 (464 mg, 1.43 mmol), 4.0 M aq. LiOH (2.0 mL, 7.17 mmol) in MeOH (4 mL) and H₂O (0.8 mL). After workup, the intermediate 10.6 was obtained as a white solid (477 mg, 1.43 mmol). Yield: quantitative. MS-ESI(−) m/z: 308.5 (M−H).

### Example 11: (±)-trans-1-(tert-Butoxycarbonyl)-4-(tetrahydro-2H-pyran-4-yl)pyrrolidine-3-carboxylic acid (11.6)

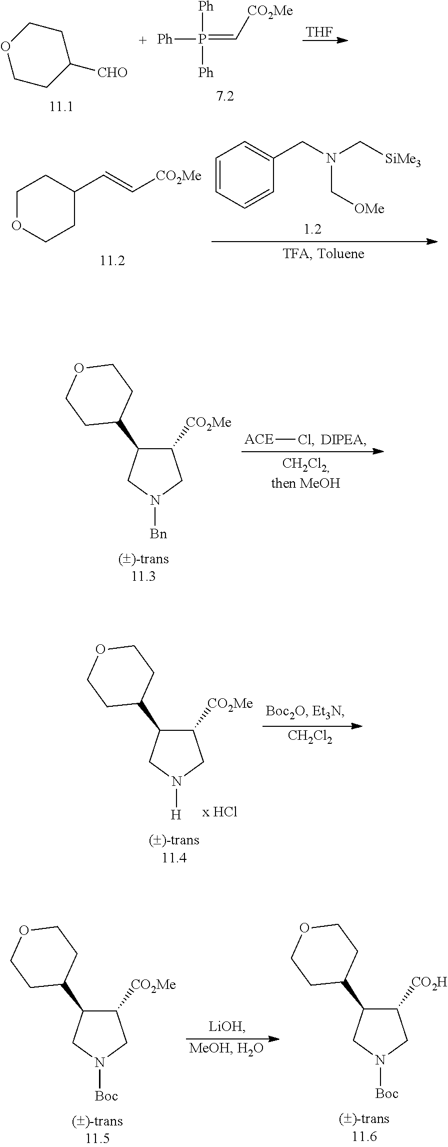

### Step 1: Methyl (2E)-3-(tetrahydro-2H-pyran-4-yl)prop-2-enoate (11.2)

Intermediate 11.2 was synthesized according to the procedure described in Step 1 of Example 7 from intermediate 11.1 (500 mg, 4.38 mmol) and intermediate 7.2 (1.70 g, 4.88 mmol) in THF (20 mL). The intermediate 11.2 (602 mg, 3.54 mmol) was obtained as a colorless oil after chromatographic purification (PET/EtOAc, from 100% PET to 90:10, v/v). Yield: 81%. MS-ESI(−) m/z: 169.5 (M−H).

### Step 2: Methyl (±)-trans-1-benzyl-4-(tetrahydro-2H-pyran-4-yl)pyrrolidine-3-carboxylate (11.3)

Intermediate 11.3 was synthesized according to the procedure described in Step 1 of Example 1 from intermediate 11.2 (600 mg, 3.53 mmol), intermediate 1.2 (1.17 mL, 4.58 mmol) and TFA (0.02 mL, 0.35 mmol) in CH₂Cl₂ (10 mL). The intermediate 11.3 (1.01 g, 3.33 mmol) was obtained after work-up and chromatographic purification (PET/EtOAc, from 100% PET to 90:10, v/v). Yield: 94%. MS-ESI(+) m/z: 304.0 (M+H).

### Step 3: 1-tert-Butyl 3-methyl (±)-trans-4-(tetrahydro-2H-pyran-4-yl)pyrrolidine-1,3-dicarboxylate (11.5)

Intermediate 11.5 was synthesized according to the procedure described in Step 2 of Example 1 from intermediate 11.3 (1.00 g, 3.30 mmol), DIPEA (0.63 mL, 3.63 mmol) and 1-chloroethylchloroformate (0.89 mL, 8.25 mmol) in CH₂Cl₂ (25 mL). The obtained crude was treated in refluxing MeOH (15 mL). After removal of volatiles, the intermediate 11.4 was reacted with Boc₂O (1.08 g, 4.95 mmol) and DIPEA (1.72 mL, 9.90 mmol) in CH₂Cl₂ (25 mL). After work-up and chromatographic purification (PET/EtOAc, from 100% PET to 80:20, v/v), the intermediate 11.5 (0.80 g, 2.55 mmol) was obtained in 78% yield. MS-ESI(+) m/z 314.5 (M+H).

### Step 4: (±)-trans-1-(tert-Butoxycarbonyl)-4-(tetrahydro-2H-pyran-4-yl)pyrrolidine-3-carboxylic acid (11.6)

Intermediate 11.6 was synthesized according to the procedure described in Step 3 of Example 6 from intermediate 11.5 (800 mg, 2.55 mmol), aq. LiOH 4.0 M (3.6 mL, 14.4 mmol) in MeOH (5 mL) and H₂O (1 mL). After workup, the title intermediate 11.6 was obtained as a white solid (0.65 g, 2.19 mmol). Yield: 86%. MS-ESI(−) m/z: 298.5 (M−H).

### Example 12: (±)-trans-1-(tert-Butoxycarbonyl)-4-(4-methoxyphenyl)pyrrolidine-3-carboxylic acid (12.6)

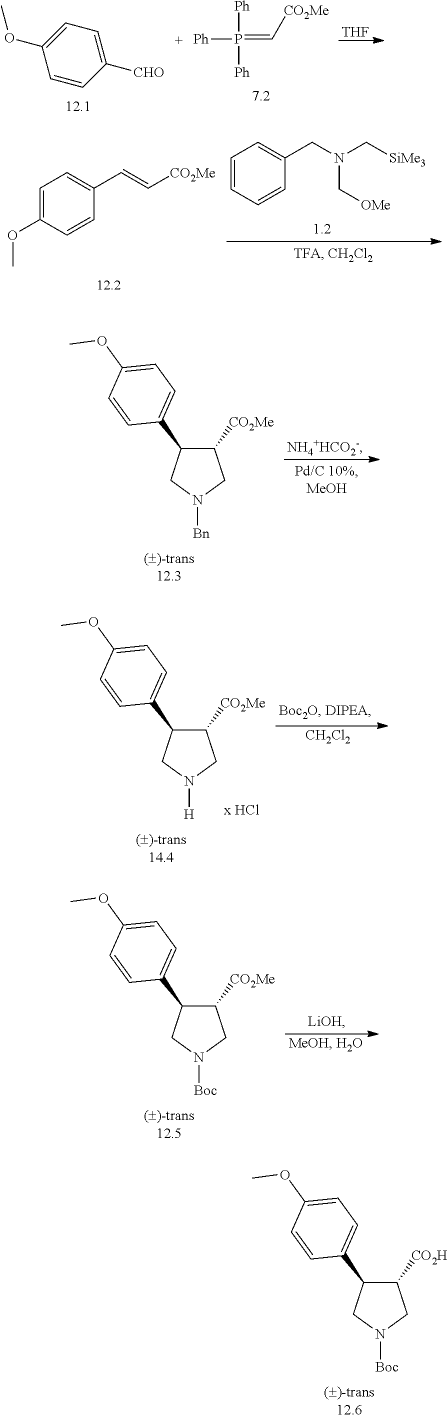

### Step 1: Methyl (2E)-3-(4-methoxyphenyl)prop-2-enoate (12.2)

Intermediate 12.2 was synthesized according to the procedure described in Step 1 of Example 7 from intermediate 12.1 (0.36 mL, 2.94 mmol) and intermediate 7.2 (1.23 g, 3.67 mmol) in THF (10 mL). The intermediate 12.2 (317 mg, 1.65 mmol) was obtained as white crystals after chromatographic purification (PET/EtOAc, from 95:5 to 80:20, v/v). Yield: 68%. MS-ESI(−) m/z: 191.2 (M−H).

### Step 2: Methyl (±)-trans-1-benzyl-4-(4-methoxyphenyl)pyrrolidine-3-carboxylate (12.3)

Intermediate 12.3 was synthesized according to the procedure described in Step 1 of Example 1 from intermediate 12.2 (306 mg, 1.59 mmol), intermediate 1.2 (0.53 mL, 2.07 mmol) and TFA (0.012 mL, 0.16 mmol) in CH₂Cl₂ (3.5 mL). The intermediate 12.3 (337 mg, 1.04 mmol) was obtained after work-up and chromatographic purification (PET/EtOAc, from 100% PET to PET/EtOAc 8:2 v/v). Yield: 65%. MS-ESI(−) m/z: 326.5 (M−H).

### Step 3: 1-tert-Butyl 3-methyl (±)-trans-4-(4-methoxyphenyl)pyrrolidine-1,3-dicarboxylate (12.5)

Intermediate 12.5 was synthesized according to the procedure described in Step 3 of Example 8 from intermediate 12.3 (330 mg, 1.01 mmol), Pd/C 10% (40 mg), ammonium formate (183 mg, 3.04 mmol) in MeOH (10 mL). After filtration, the liquor containing the intermediate 12.4 was treated with Et₃N (0.71 mL, 5.07 mmol) and Boc₂O (664 mg, 3.04 mmol). The title intermediate 12.5 (333 mg, 0.98 mmol) was obtained after work-up and chromatographic purification (PET/EtOAc, from 95:5 to 80:20, v/v). Yield: 98%. MS-ESI(+) m/z 336.5 (M+H).

### Step 4: (±)-trans-1-(tert-Butoxycarbonyl)-4-(4-methoxyphenyl)pyrrolidine-3-carboxylic acid (12.6)

Intermediate 12.6 was synthesized according to the procedure described in Step 3 of Example 6 from intermediate 12.5 (327 mg, 0.97 mmol), 4.0 M aq. LiOH (1.5 mL, 4.87 mmol) in MeOH (3 mL) and H2O (0.7 mL). After workup, the intermediate 12.6 was obtained as a white solid (244 mg, 0.76 mmol). Yield: 78%. MS-ESI(−) m/z: 320.4 (M−H).

### Example 13: (±)-trans-1-(tert-Butoxycarbonyl)-4-cyclohexyl-pyrrolidine-3-carboxylic acid (13.1)

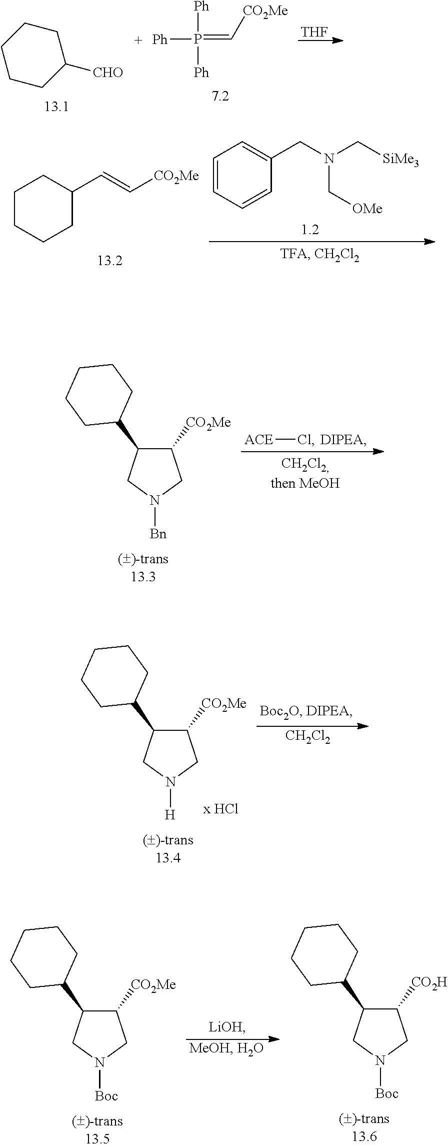

### Step 1: Methyl (2E)-3-cyclohexylprop-2-enoate (13.2)

Intermediate 13.2 was synthesized according to the procedure described in Step 1 of Example 7 from intermediate 13.1 (1.08 mL, 8.92 mmol) and intermediate 7.2 (3.72 g, 11.14 mmol) in THF (15 mL). The intermediate 13.2 (1.18 g, 7.01 mmol) was obtained as a colorless oil after chromatographic purification (PET/EtOAc, isocratic 95:5, v/v). Yield: 78%. MS-ESI(−) m/z: 167.4 (M−H).

### Step 2: Methyl (±)-trans-1-benzyl-4-(cyclohexyl)pyrrolidine-3-carboxylate (13.3)

Intermediate 13.3 was synthesized according to the procedure described in Step 1 of Example 1 from intermediate 13.2 (1.15 g, 6.84 mmol), intermediate 1.2 (1.92 mL, 7.51 mmol), and TFA (0.16 mL, 2.05 mmol) in CH₂Cl₂ (30 mL). The intermediate 13.3 (765 mg, 2.54 mmol) was obtained after work-up and chromatographic purification (PET/EtOAc, from 100% PET to 90:10, v/v). Yield: 37%. MS-ESI(−) m/z: 300.6 (M−H).

### Step 3: 1-tert-Butyl 3-methyl (±)-trans-4-(cyclohexyl)pyrrolidine-1,3-dicarboxylate (13.5)

Intermediate 13.5 was synthesized according to the procedure described in Step 2 of Example 1 from intermediate 13.3 (745 mg, 2.47 mmol), DIPEA (0.47 mL, 2.72 mmol), and 1-chloroethylchloroformate (0.67 mL, 6.18 mmol) in CH₂Cl₂ (10 mL). The obtained crude was treated in refluxing MeOH (10 mL). After removal of volatiles, the intermediate 13.4 was reacted with Boc₂O (593 mg, 2.72 mmol) and DIPEA (1.29 mL, 5.77 mmol) in CH₂Cl₂ (10 mL). After work-up the crude of intermediate 13.5 (1.0 g) was used such without purification. MS-ESI(+) m/z 312.3 (M+H).

### Step 4: (±)-trans-1-(tert-Butoxycarbonyl)-4-(cyclohexyl)pyrrolidine-3-carboxylic acid (13.6)

The title intermediate 13.6 was synthesized according to the procedure described in Step 3 of Example 6 from the crude of intermediate 13.5 (2.47 mmol), 4.0 M aq. LiOH (3.0 mL, 12.34 mmol) in MeOH (15 mL), and H₂O (5 mL). Yield: 82% from 11.3. MS-ESI(−) m/z: 296.4 (M−H).

### Example 14: (±)-trans-1-(tert-Butoxycarbonyl)-4-benzyl-pyrrolidine-3-carboxylic acid (14.1)

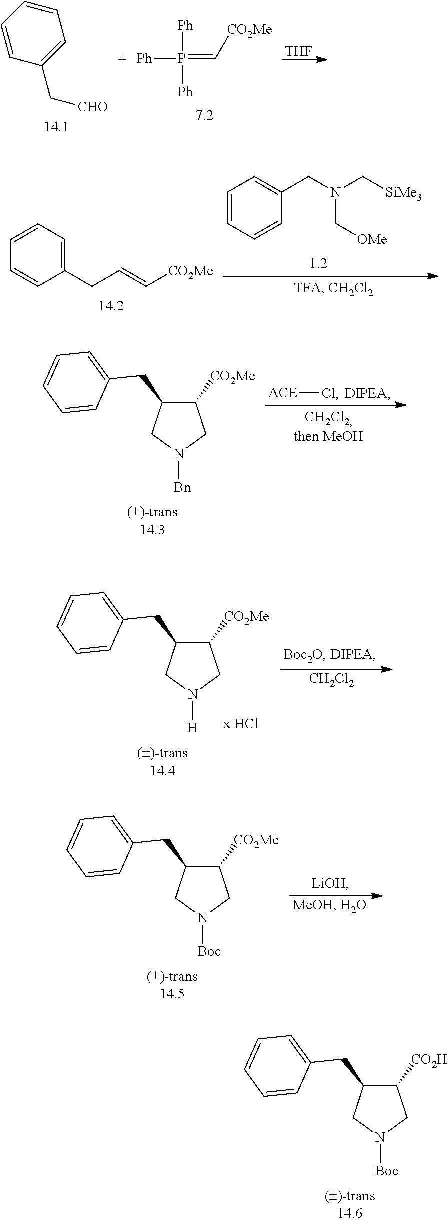

### Step 1: Methyl (2E)-4-phenylbut-2-enoate (14.2)

Intermediate 14.2 was synthesized according to the procedure described in Step 1 of Example 7 from intermediate 14.1 (0.93 mL, 8.33 mmol) and intermediate 7.2 (3.48 g, 10.41 mmol) in THF (15 mL). The intermediate 14.2 (1.05 g) was obtained as a colorless oil after chromatographic purification (PET/EtOAc, from 95:5 to 80:20, v/v). Yield: 57%. MS-ESI(−) m/z: 175.2 (M−H).

### Step 2: Methyl (±)-trans-1,4-dibenzylpyrrolidine-3-carboxylate (14.3)

Intermediate 14.3 was synthesized according to the procedure described in Step 1 of Example 1 from intermediate 14.2 (1.02 g, 5.79 mmol), intermediate 1.2 (1.63 mL, 6.37 mmol) and TFA (0.13 mL, 1.74 mmol) in CH₂Cl₂ (20 mL). The intermediate 14.3 (860 mg, 2.78 mmol) was obtained after work-up and chromatographic purification (PET/EtOAc, from PET 100% to 80:20, v/v). Yield: 48%. MS-ESI(−) m/z: 308.5 (M−H).

### Step 3: 1-tert-Butyl 3-methyl (±)-trans-4-benzylpyrrolidine-1,3-dicarboxylate (14.5)

Intermediate 14.5 was synthesized according to the procedure described in Step 2 of Example 1 from intermediate 14.3 (840 mg, 2.72 mmol), DIPEA (0.52 mL, 2.99 mmol) and 1-chloroethylchloroformate (0.73 mL, 6.79 mmol) in CH₂Cl₂ (10 mL). The obtained crude was treated in refluxing MeOH (10 mL). After removal of volatiles, the intermediate 14.4 was reacted with Boc₂O (651 mg, 2.99 mmol), and DIPEA (1.42 mL, 8.15 mmol) in CH₂Cl₂ (10 mL). After work-up the crude of intermediate 14.5 (1.1 g) was used such without purification. MS-ESI(+) m/z 320.3 (M+H).

### Step 4: (±)-trans-4-Benzyl-1-(tert-butoxycarbonyl)pyrrolidine-3-carboxylic acid (14.6)

The title intermediate 14.6 was synthesized according to the procedure described in Step 3 of Example 6 from the crude of intermediate 14.5 (2.72 mmol) and 4.0 M aq. LiOH (3.4 mL, 13.58 mmol) in MeOH (15 mL) and H₂O (5 mL). Yield: 97% from 2.3. MS-ESI(−) m/z: 304.8 (M−H).

### Example 15: (±)-trans-4-Phenyl-1-(tetrahydro-2H-pyran-4-yl)pyrrolidine-3-carboxylic acid hydrochloride (15.3)

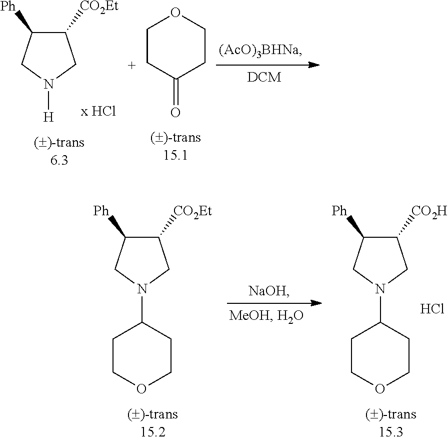

### Step 1: Ethyl (±)-trans-4-phenyl-1-(tetrahydro-2H-pyran-4-yl)pyrrolidine-3-carboxylate (15.2)

The crude coming from methanolysis of Step 2 Example 6 was concentrated under reduced pressure. The resulting intermediate 6.3 (0.57 g, 2.26 mmol) was dissolved in CH₂Cl₂ (30 mL) and reacted with intermediate 15.1 (0.63 mL, 6.79 mmol) in the presence of sodium triacetoxyborohydride (1.92 g, 9.05 mmol). The resulting mixture was stirred at r.t. for 18 h, and then poured into aq. NaHCO₃ ss (20 mL). The two phases were separated and the aqueous one was extracted with CH₂Cl₂ (2×30 mL). The collected organic layers were washed with brine (50 mL), dried over Na₂SO₄, and concentrated under reduced pressure. After chromatographic purification (CH₂Cl₂/MeOH from 99:1 to 94:6 v/v), 630 mg (2.08 mmol) of intermediate 15.2 were obtained as a yellow oil (yield: 92%). MS-ESI(−) m/z 302.4 (M−H).

### Step 2: (±)-trans-4-Phenyl-1-(tetrahydro-2H-pyran-4-yl)pyrrolidine-3-carboxylic acid hydrochloride (15.3)

5.0 M aq. NaOH (2.0 mL, 10.05 mmol) was added to a solution of intermediate 15.2 (610 mg, 2.01 mmol) in MeOH (10 mL), and the mixture was stirred at r.t. for 16 h. Volatiles were removed under reduced pressure and the crude was dissolved in H₂O (8 mL) and acidified up to pH=4.0 by adding 3.0 M HCl. The solution was washed with CH₂Cl₂ (3×5 mL) and concentrated under reduced pressure. The resulting solid was suspended in MeOH (5 mL) and filtered under vacuum. The collected liquor was concentrated under reduced pressure, to give the title intermediate 15.3 in nearly quantitative yield (548 mg, 1.99 mmol). MS-ESI(−) m/z 274.6 (M−H).

### Example 16: (±)-trans-1-Acetyl-4-phenylpyrrolidine-3-carboxylic acid (16.2)

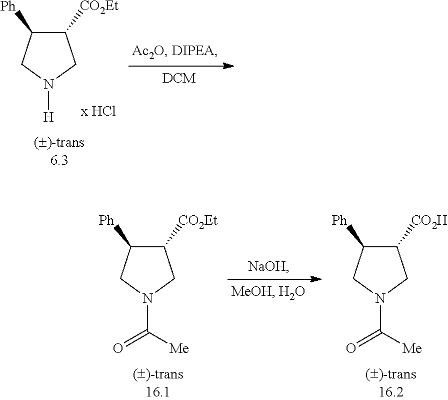 [SMILES: CCOC(=O)[C@@H]1CNC[C@H]1c1ccccc1 ; CCOC(=O)[C@@H]1CN(C(C)=O)C[C@H]1c1ccccc1 ; CC(=O)N1C[C@@H](C(=O)O)[C@H](c2ccccc2)C1]

### Step 1: Ethyl (±)-trans-1-acetyl-4-phenylpyrrolidine-3-carboxylate (16.1)

The crude coming from methanolysis of Step 2 Example 6 was concentrated under reduced pressure. The resulting intermediate 6.3 (0.57 g, 2.26 mmol) was dissolved in CH₂Cl₂ (30 mL) and reacted with acetic anhydride (0.32 mL, 3.39 mmol) and DIPEA (1.18 mL, 6.79 mmol) in CH₂Cl₂ (15 mL) for 3 h. The mixture was washed with 0.5 M aq. citric acid (15 mL), brine (15 mL), dried over Na₂SO₄, and concentrated under reduced pressure. After chromatographic purification (CH₂Cl₂/MeOH from 99:1 to 95:5 v/v), 0.59 g (2.26 mmol) of intermediate 16.1 were obtained as a yellow oil (quantitative yield). MS-ESI(−) m/z 260.5 (M−H).

### Step 2: (±)-trans-1-Acetyl-4-phenylpyrrolidine-3-carboxylic acid (16.2)

NaOH (488 mg, 12.21 mmol) was added to a stirred solution of intermediate 16.1 (638 mg, 2.44 mmol) in MeOH (15 mL), and the mixture was stirred at r.t. for 18 h. Volatiles were removed under reduced pressure, and the crude was dissolved in H₂O (10 mL). The aqueous solution was acidified up to pH=1 by adding 37% HCl and then extracted with CH₂Cl₂/MeOH (9:1, v/v, 3×15 mL). The collected organic phase was dried over Na₂SO₄ and concentrated under reduced pressure, to give 540 mg (2.32 mmol, yield: 95%) of the title intermediate 16.2. MS-ESI(−) m/z 232.5 (M−H).

### Example 17: (3S,4R)-1-(tert-Butoxycarbonyl)-4-phenylpyrrolidine-3-carboxylic acid (17.6)

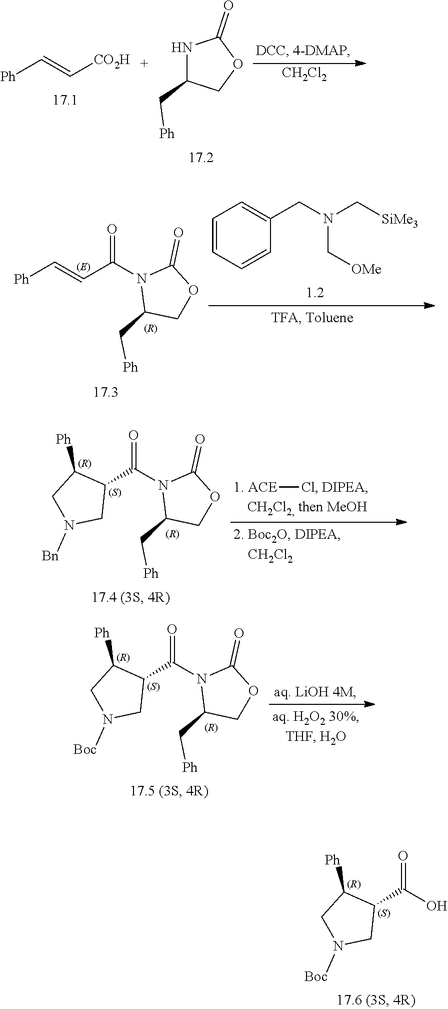

### Step 1: (4R)-4-Benzyl-3-[(2E)-3-phenylprop-2-enoyl]-1,3-oxazolidin-2-one (17.3)

DCC (13.98 g, 67.79 mmol) was added to a stirred solution of trans-cinnamic acid (10.00 g, 67.79 mmol), intermediate 17.2 (9.20 g, 51.92 mmol), and DMAP (0.83 g, 6.78 mmol) in CH₂Cl₂ (80 mL) cooled at 0-5° C., and the mixture was stirred at room temperature for 18 h. The obtained suspension was filtered under vacuum and the solid washed with CH₂Cl₂ (30 mL). The collected liquors were washed with 10% aq. NaHCO₃ (50 mL), brine (50 mL), dried over Na₂SO₄, and concentrated under reduced pressure. The crude was purified by flash chromatography (PET/EtOAc, from 95:5 to 60:40 v/v), to afford the intermediate 17.3 (16.46 g, 53.55 mmol) in 79% yield. MS-ESI(−) m/z 306.3 (M−H).

### Step 2: (4R)-Benzyl-3-[(3S,4R)1-benzyl-4-phenyl-pyrrolidine-3-carbonyl]-oxazolidin-2-one (17.4)

Intermediate 1.2 (49 mL, 0.19 mol) and TFA (3.67 mL, 47.87 mmol) were added to a stirred solution of intermediate 17.3 (49.00 g, 0.159 mol) in toluene (350 mL) cooled at 0-5° C., and the resulting mixture was stirred at r.t. for 18 h. 10% aq. NaHCO₃ (350 mL) was cautiously added, the two phases were separated and the organic phase was washed with brine (250 mL), dried over Na₂SO₄, and concentrated under reduced pressure. The crude was purified by flash chromatography (PET/EtOAc, from 90:10 to 50:50). The first eluate is the diasteroisomer 17.4, isolated in 51% yield (35.72 g, 81.09 mmol) as a whitish solid. MS-ESI(+) m/z 441.3 (M+H).

### Step 3: tert-Butyl (3S,4R)-3-[(4R)-benzyl-2-oxo-oxazolidine-3-carbonyl]-4-phenyl-pyrrolidine-1-carboxylate (17.5)

DIPEA (4.35 mL, 24.97 mmol) and 1-chloroethylchloroformate (6.12 mL, 56.75 mmol) were added to a stirred solution of intermediate 17.4 (10.00 g, 22.70 mmol) in CH₂Cl₂ (120 mL), and the resulting mixture was stirred and refluxed for 1 h. Once cooled to r.t., the volatiles were removed under reduced pressure. The crude was dissolved in MeOH (100 mL) and vigorously stirred and refluxed for 1 h. The reaction was cooled to r.t. and concentrated under reduced pressure. The obtained residue was treated with Boc₂O (5.45 g, 24.97 mmol) and DIPEA (11.86 mL, 68.10 mmol) in CH₂Cl₂ (100 mL) at r.t. for 3 h. The mixture was washed with 0.5 M aq. citric acid (2×50 mL), 10% aq. NaHCO₃ (80 mL), brine (280 mL), dried over Na₂SO₄, and concentrated under reduced pressure. The obtained crude of intermediate 17.5 was used such as for the next step. MS-ESI(+) m/z 351.3 (M+H-100).

### Step 4: (3S,4R)-1-(tert-Butoxycarbonyl)-4-phenylpyrrolidine-3-carboxylic acid (17.6)

4.0 M aq. LiOH (22.7 mL, 90.80 mmol), and 30% aq. H₂O₂ (23.2 mL, 0.23 mol) were added to a stirred solution of intermediate 17.5 (crude of previous step, 22.70 mmol) in THF (120 mL) and H₂O (20 mL), and the reaction was stirred at r.t. for 4 h. The mixture was concentrated under reduced pressure to % of the initial volume, then poured into H₂O (50 mL), and washed with EtOAc (3×30 mL). The aqueous phase was acidified with 3.0 M HCl up to pH=2.5 and then extracted with CH₂Cl₂ (3×50 mL). The collected organic layers were washed with brine (50 mL), dried over Na₂SO₄ and concentrated under reduced pressure. The title intermediate 17.6 was obtained as a white powder (6.01 g, 20.63 mmol, yield: 91%). MS-ESI(−) m/z 290.1 (M−H).

### Example 18: (3R,4S)-1-(tert-Butoxycarbonyl)-4-phenylpyrrolidine-3-carboxylic acid (18.3)

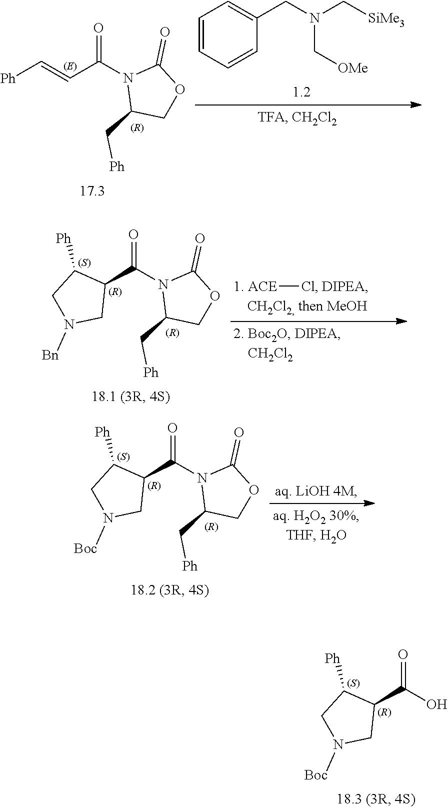

### Step 1: (4R)-Benzyl-3-[(3R,4S)1-benzyl-4-phenyl-pyrrolidine-3-carbonyl]-oxazolidin-2-one (18.1)

Intermediate 1.2 (49 mL, 0.19 mol) and TFA (3.67 mL, 47.87 mmol) were added to a stirred solution of intermediate 17.3 (49.00 g, 0.159 mol) in toluene (350 mL) cooled at 0-5° C., and the resulting mixture was stirred at r.t. for 18 h. 10% aq. NaHCO₃ (350 mL) was cautiously added, the two phases were separated and the organic one was washed with brine (250 mL), dried over Na₂SO₄, and concentrated under reduced pressure. The crude was purified by flash chromatography (PET/EtOAc, from 90:10 to 50:50). The second eluate is the diasteroisomer 18.1, isolated in 46% yield (32.65 g, 74.12 mmol) as a pale yellow oil. MS-ESI(+) m/z 441.3 (M+H).

### Step 2: tert-Butyl (3R,4S)-3-[(4R)-benzyl-2-oxo-oxazolidine-3-carbonyl]-4-phenyl-pyrrolidine-1-carboxylate (18.2)

To a stirred solution of intermediate 18.1 (32.60 g, 74.01 mmol) in CH₂Cl₂ (400 mL), DIPEA (14.20 mL, 81.41 mmol) and 1-chloroethylchloroformate (19.96 mL, 0.19 mol) were added, and the resulting mixture was stirred and refluxed for 1 h. Once cooled to r.t., volatiles were removed under reduced pressure. The crude was dissolved in MeOH (400 mL) and vigorously stirred at reflux for 1 h. The reaction was cooled to r.t. and concentrated under reduced pressure. The residue was triturated with cold acetone (250 mL), and the solid collected by filtration under vacuum. The obtained solid was treated with Boc₂O (18.69 g, 81.41 mmol) and DIPEA (38.66 mL, 0.22 mol) in CH₂Cl₂ (350 mL) and stirred at r.t. for 3 h. The mixture was washed with 0.5 M aq. citric acid (2×200 mL), 10% aq. NaHCO₃ (250 mL), brine (250 mL), dried over Na₂S04, and concentrated under reduced pressure, to give 33.20 g (73.74 mmol) of title intermediate 18.2 as a vitreous pale yellow solid (nearly quantitative yield). MS-ESI(+) m/z 351.3 (M+H-100).

### Step 3: (3R,4S)-1-(tert-Butoxycarbonyl)-4-phenylpyrrolidine-3-carboxylic acid (18.3)

The intermediate 18.3 was synthesized according to the procedure reported in Step 3 of Example 17, from intermediate 18.2 (33.20 g, 73.69 mmol), 4.0 M aq. LiOH (74 mL, 0.296 mol), and 30% aq. H₂O₂ (75 mL, 0.74 mol) in THF (350 mL) and H₂O (60 mL). After work-up, the title intermediate 18.3 was obtained as a white powder (19.52 g, 67.05 mmol, yield: 91%). MS-ESI(−) m/z 290.1 (M−H).

### Example 19: (3R,4R)-1-(tert-Butoxycarbonyl)-4-(thiophen-2-yl)pyrrolidine-3-carboxylic acid (19.5)

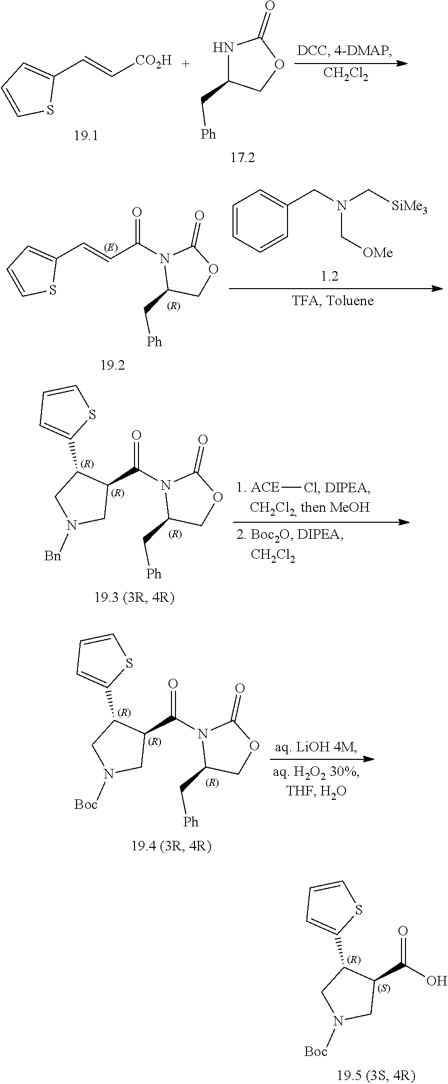

### Step 1: (4R)-4-Benzyl-3-[(2E)-3-(thiophen-2-yl)prop-2-enoyl]-1,3-oxazolidin-2-one (19.2)

The intermediate 19.2 was synthesized according to the experimental procedure of Step 1 of Example 17, starting from intermediate 19.1 (1.70 g, 11.02 mmol), intermediate 17.2 (1.69 g, 9.58 mmol), DMAP (0.15 g, 1.24 mmol), and DCC (2.37 g, 11.49 mmol) in CH₂Cl₂ (20 mL). After work up and chromatographic purification, 3.01 g (9.61 mmol) of intermediate 19.2 were obtained. Yield: 87%. MS-ESI(+) m/z 314.6 (M+H).

### Step 2: (4R)-Benzyl-3-[(3R,4R)1-benzyl-4-(thiophen-2-yl)-pyrrolidine-3-carbonyl]-oxazolidin-2-one (19.3)

The diasteroisomer 19.3 was synthesized according to the experimental procedure of Step 2 of Example 17, starting from intermediate 19.2 (3.00 g, 9.13 mmol), intermediate 1.2 (2.57 mL, 10.10 mmol), and TFA (0.12 mL, 1.64 mmol) in toluene (30 mL). After work up and chromatographic purification (PET/EtOAc, from 90:10 to 30:70, v/v), the second eluate is the diasteroisomer 19.3 (2.00 g, 4.47 mmol). Yield: 49%. MS-ESI(+) m/z 447.4 (M+H).

### Step 3: tert-Butyl (3R,4R)-3-[(4R)-benzyl-2-oxo-oxazolidine-3-carbonyl]-4-(thiophen-2-yl)-pyrrolidine-1-carboxylate (19.4)

Intermediate 19.4 was synthesized according to the procedure described in Step 2 of Example 1 from intermediate 19.3 (2.00 g, 4.47 mmol), DIPEA (0.86 mL, 4.91 mmol), and 1-chloroethylchloroformate (1.20 mL, 11.20 mmol) in CH₂Cl₂ (100 mL). The obtained crude was treated in refluxing MeOH (100 mL). After removal of volatiles, the crude was triturated in cold acetone (30 mL), and the solid filtered under vacuum. The pure collected debenzylated intermediate (0.94 g, 2.39 mmol) was reacted with Boc₂O (0.78 g, 3.58 mmol) and DIPEA (1.25 mL, 7.17 mmol) in CH₂Cl₂ (25 mL). After work-up the crude of intermediate 19.4 (2.2 g) was used such as for the next step. MS-ESI(+) m/z 457.8 (M+H).

### Step 4: (3R,4R)-1-(tert-Butoxycarbonyl)-4-(thiophen-2-yl)-pyrrolidine-3-carboxylic acid (19.5)

The intermediate 19.5 was synthesized according to the procedure reported in Step 4 of Example 17, from intermediate 19.4 (crude of previous step, 4.47 mmol), 4.0 M aq. LiOH (4.47 mL, 17.88 mol), and 30% aq. H₂O₂ (4.56 mL, 44.70 mol) in THF (50 mL) and H₂O (12 mL). After work-up, the title intermediate 19.5 was obtained as a white powder in nearly quantitative yield (1.33 g, 4.47 mmol) from 9.3. MS-ESI(−) m/z 296.6 (M−H).

### Example 20: (3S,4S)-1-(tert-Butoxycarbonyl)-4-(thiophen-2-yl)pyrrolidine-3-carboxylic acid (20.3)

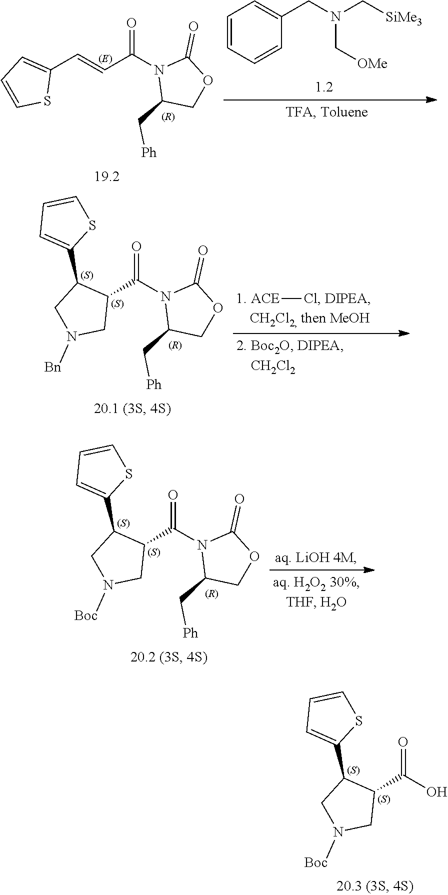

### Step 1: (4R)-Benzyl-3-[(3S,4S)1-benzyl-4-(thiophen-2-yl)-pyrrolidine-3-carbonyl]-oxazolidin-2-one (20.1)

The diasteroisomer 20.1 was synthesized according to the experimental procedure of Step 2 of Example 17, starting from intermediate 19.2 (3.00 g, 9.13 mmol), intermediate 1.2 (2.57 mL, 10.10 mmol), and TFA (0.12 mL, 1.64 mmol) in toluene (30 mL). After work up and chromatographic purification (PET/EtOAc, from 90:10 to 70:30, v/v), the first eluate is the diasteroisomer 20.1 (1.81 g, 4.05 mmol). Yield: 44%. MS-ESI(+) m/z 447.4 (M+H).

### Step 2: tert-Butyl (3S,4S)-3-[(4R)-benzyl-2-oxo-oxazolidine-3-carbonyl]-4-(thiophen-2-yl)-pyrrolidine-1-carboxylate (20.2)

Intermediate 20.2 was synthesized according to the procedure described in Step 2 of Example 1 from intermediate 20.1 (1.80 g, 4.03 mmol), DIPEA (0.77 mL, 4.43 mmol), and 1-chloroethylchloroformate (1.09 mL, 10.08 mmol) in CH₂Cl₂ (30 mL). The obtained crude was treated in refluxing MeOH (15 mL). After removal of volatiles, the debenzylated intermediate was reacted with Boc₂O (1.32 mg, 6.05 mmol) and DIPEA (2.10 mL, 12.09 mmol) in CH₂Cl₂ (30 mL). After work-up, the crude was purified by flash chromatography (PET/EtOAc, from 90:10 to 60:40, v/v), to afford 1.77 g (3.88 mmol) of intermediate 20.2 as a pale yellow oil. Yield: 96%. MS-ESI(+) m/z 357.3 (M+H-100).

### Step 3: (3S,4S)-1-(tert-Butoxycarbonyl)-4-(thiophen-2-yl)-pyrrolidine-3-carboxylic acid (20.3)

The intermediate 20.3 was synthesized according to the procedure reported in Step 4 of Example 17, from intermediate 20.2 (1.75 g, 3.83 mmol), 4.0 M aq. LiOH (3.8 mL, 15.33 mol), and 30% aq. H₂O₂ (5.8 mL, 57.50 mol) in THF (30 mL) and H₂O (7.5 mL). After work-up, the title intermediate 20.3 was obtained as a white powder (880 mg, 2.96 mmol, yield: 77%). MS-ESI(−) m/z 296.2 (M−H).

### Example 21: (3R,4R)-1-(tert-Butoxycarbonyl)-4-(1,3-thiazol-2-yl)pyrrolidine-3-carboxylic acid (21.7)

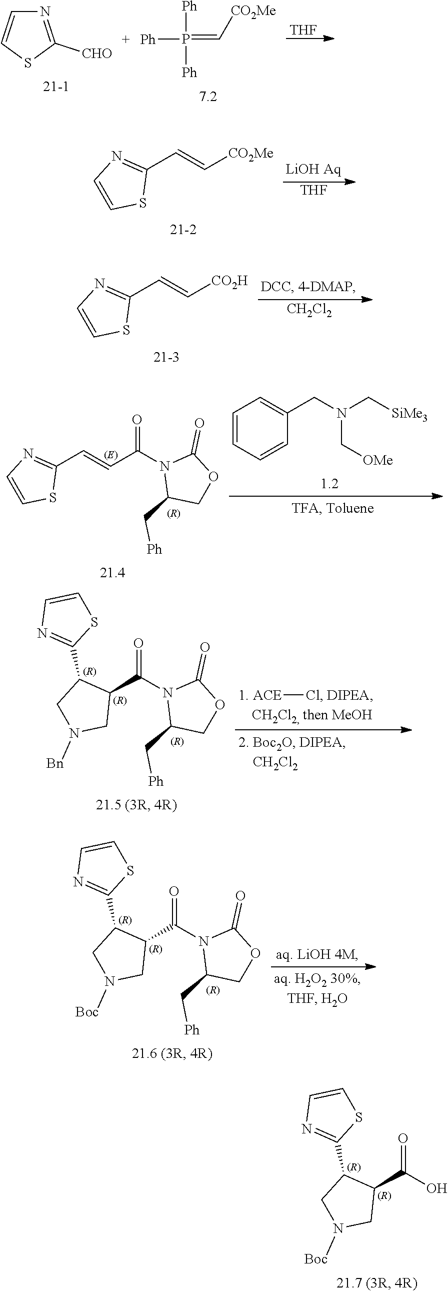

### Step 1: Methyl (2E)-3-(1,3-thiazol-2-yl)prop-2-enoate (21.2)

The intermediate 21.2 was synthesized according to the procedure described in Step 1 of Example 7 starting from intermediate 21.1 (0.39 mL, 4.42 mmol) and intermediate 7.2 (1.72 g, 4.95 mmol) in THF (15 mL). The intermediate 21.2 (705 mg, 4.17 mmol) was obtained as white crystals after chromatographic purification (PET/EtOAc, from 90:1 to 70:30, v/v). Yield: 94%. MS-ESI(−) m/z: 170.4 (M−H).

### Step 2: (2E)-3-(1,3-Thiazol-2-yl)prop-2-enoic acid (21.3)

Aq. LiOH 1.0 M (4.55 mL, 4.55 mmol) was added to a stirred solution of 21.2 (700 mg, 4.13 mmol) in THF (20 mL), and the mixture was stirred at r.t. for 3 h. The reaction was then poured into H₂O (20 mL) and acidified up to pH=1 by adding 1.0 M HCl. The aqueous phase was extracted with EtOAc (3×20 mL), and the collected organic layers were washed with brine (30 mL), dried over Na₂SO₄, and concentrated under reduced pressure. 580 mg (3.74 mmol) of intermediate 21.3 were obtained as a white powder. Yield: 72%. MS-ESI(−) m/z: 154.5 (M−H).

### Step 3: (4R)-4-Benzyl-3-[(2E)-3-(1,3-thiazol-2-yl)prop-2-enoyl]-1,3-oxazolidin-2-one (21.4)

The intermediate 21.4 was synthesized according to the experimental procedure of Step 1 of Example 17, starting from intermediate 21.3 (565 mg, 3.64 mmol), intermediate 7.2 (568 mg, 3.30 mmol), DMAP (52 mg, 0.42 mmol), and DCC (0.90 g, 4.36 mmol) in CH₂Cl₂ (15 mL). After work up and chromatographic purification, 1.03 g (3.28 mmol) of intermediate 21.4 were obtained. Yield: 90%. MS-ESI(+) m/z 315.5 (M+H).

### Step 4: (4R)-Benzyl-3-[(3R,4R)1-benzyl-4-(1,3-thiazol-2-yl)-pyrrolidine-3-carbonyl]-oxazolidin-2-one (21.5)

The diasteroisomer 21.5 was synthesized according to the experimental procedure of Step 2 of Example 17, starting from intermediate 21.4 (1.00 g, 3.18 mmol), intermediate 1.2 (0.89 mL, 3.49 mmol), and TFA (0.04 mL, 0.57 mmol) in toluene (10 mL). After work up and chromatographic purification (PET/AcOEt, from 80:20 to 30:70, v/v), the second eluate is the diasteroisomer 21.5 (0.74 g, 1.65 mmol). Yield: 52%. MS-ESI(+) m/z 448.6 (M+H).

### Step 5: tert-Butyl (3R,4R)-3-[(4R)-benzyl-2-oxo-oxazolidine-3-carbonyl]-4-(1,3-thiazol-2-yl)-pyrrolidine-1-carboxylate (21.6)

Intermediate 21.6 was synthesized according to the procedure described in Step 2 of Example 1 from intermediate 21.5 (700 mg, 1.56 mmol), DIPEA (0.29 mL, 3.91 mmol), and 1-chloroethylchloroformate (0.41 mL, 3.91 mmol) in CH₂Cl₂ (30 mL). The obtained crude was treated in refluxing MeOH (30 mL). After removal of volatiles, the debenzylated intermediate was reacted with Boc₂O (510 mg, 2.34 mmol) and DIPEA (0.82 mL, 4.68 mmol) in CH₂Cl₂ (20 mL). After work-up the crude was purified by flash chromatography (PET/EtOAc, from 80:20 to 30:70, v/v), to afford 444 mg (0.97 mmol) of intermediate 21.6. Yield: 62%. MS-ESI(+) m/z 458.8 (M+H).

### Step 6: (3R,4R)-1-(tert-Butoxycarbonyl)-4-(1,3-thiazol-2-yl)-pyrrolidine-3-carboxylic acid (21.7)

The intermediate 21.7 was synthesized according to the procedure reported in Step 4 of Example 17, from intermediate 21.6 (440 mg, 0.96 mmol), 4.0 M aq. LiOH (0.96 mL, 3.84 mol), and 30% aq. H₂O₂ (0.44 mL, 14.10 mol) in THF (30 mL) and H₂O (4 mL). After work-up, the title intermediate 21.7 was obtained as a colorless oil (263 mg, 0.88 mmol, yield 92%). MS-ESI(−) m/z 297.6 (M−H).

### Example 22: (3S,4S)-1-(tert-Butoxycarbonyl)-4-(1,3-thiazol-2-yl)pyrrolidine-3-carboxylic acid (22.3)

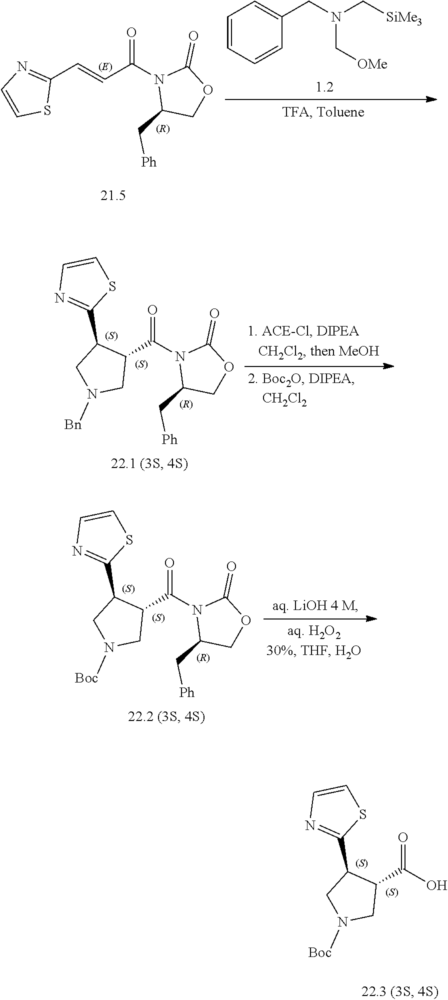

### Step 1: (4R)-Benzyl-3-[(3S,4S)1-benzyl-4-(1,3-thiazol-2-yl)-pyrrolidine-3-carbonyl]-oxazolidin-2-one (22.1)

The diasteroisomer 22.1 was synthesized according to the experimental procedure of Step 2 of Example 17, starting from intermediate 21.5 (1.00 g, 3.18 mmol), intermediate 1.2 (0.89 mL, 3.49 mmol), and TFA (0.04 mL, 0.57 mmol) in toluene (10 mL). After work up and chromatographic purification (PET/EtOAc, from 80:20 to 60:40, v/v), the first eluate is the diasteroisomer 22.1 (0.51 g, 1.14 mmol). Yield: 36%. MS-ESI(+) m/z 448.7 (M+H).

### Step 2: tert-Butyl (3S,4S)-3-[(4R)-benzyl-2-oxo-oxazolidine-3-carbonyl]-4-(1,3-thiazol-2-yl)-pyrrolidine-1-carboxylate (22.2)

Intermediate 22.2 was synthesized according to the procedure described in Step 2 of Example 1 from intermediate 22.1 (0.51 g, 1.14 mmol), DIPEA (0.22 mL, 1.25 mmol) and 1-chloroethylchloroformate (0.30 mL, 2.87 mmol), in CH₂Cl₂ (20 mL). The obtained crude was treated in refluxing MeOH (20 mL). After removal of volatiles, the debenzylated intermediate was reacted with Boc₂O (370 mg, 1.71 mmol) and DIPEA (0.59 mL, 3.42 mmol) in CH₂Cl₂ (20 mL). After work-up the crude was purified by flash chromatography (PET/EtOAc, from 80:20 to 50:50, v/v), to afford 510 mg (1.11 mmol) of the intermediate 22.2. Yield: 98%. MS-ESI(+) m/z 458.4 (M+H).

### Step 3: (3S,4S)-1-(tert-Butoxycarbonyl)-4-(1,3-thiazol-2-yl)-pyrrolidine-3-carboxylic acid (22.3)

The intermediate 22.3 was synthesized according to the procedure reported in Step 4 of Example 17, from intermediate 22.2 (506 mg, 1.10 mmol), 4.0 M aq. LiOH (1.10 mL, 4.42 mol), and 30% aq. H₂O₂ (0.50 mL, 16.5 mol) in THF (28 mL) and H₂O (4.5 mL). After work-up the title intermediate 22.3 was obtained as a white powder in nearly quantitative yield (328 mg, 1.10 mmol). MS-ESI(−) m/z 297.6 (M−H).

### Example 23: (3R,4S)-1-(tert-Butoxycarbonyl)-4-(4-fluorophenyl)-pyrrolidine-3-carboxylic acid (23.5)

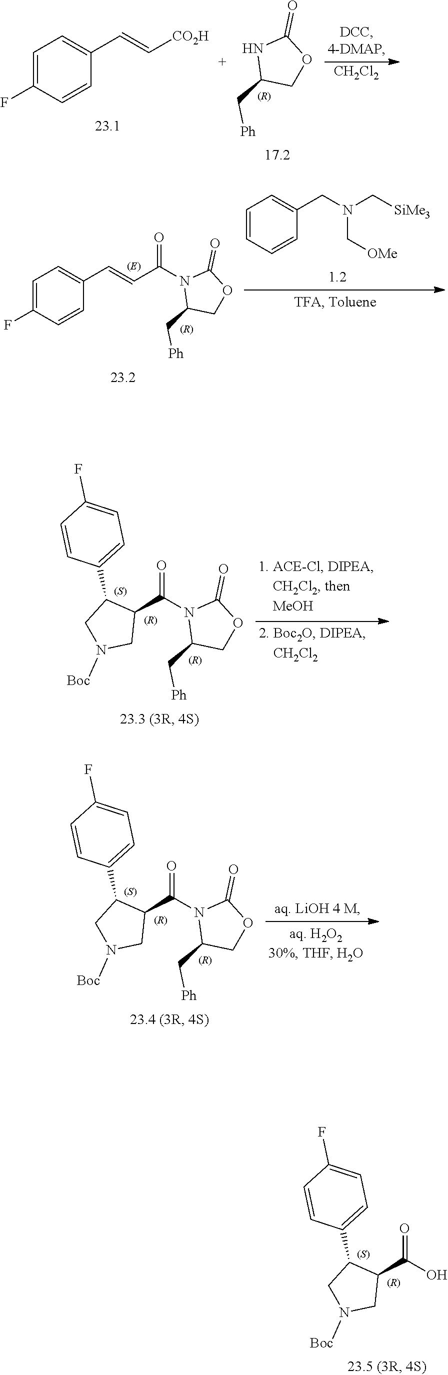

### Step 1: (4R)-4-Benzyl-3-[(2E)-3-(4-fluorophenyl)prop-2-enoyl]-1,3-oxazolidin-2-one (23.5)

The intermediate 23.2 was synthesized according to the experimental procedure of Step 1 of Example 17, starting from intermediate 23.1 (1.50 g, 9.03 mmol), intermediate 14.2 (1.45 g, 8.18 mmol), DMAP (0.13 g, 1.07 mmol), and DCC (2.03 g, 9.84 mmol) in CH₂Cl₂ (15 mL). After work up and chromatographic purification, 2.45 g (7.53 mmol) of intermediate 23.2 were obtained. Yield: 92%. MS-ESI(+) m/z 326.7 (M+H).

### Step 3: (4R)-Benzyl-3-[(3R,4S)1-benzyl-4-(4-fluorophenyl)-pyrrolidine-3-carbonyl]-oxazolidin-2-one (23.3)

The diasteroisomer 23.3 was synthesized according to the experimental procedure of Step 2 of Example 17, starting from intermediate 23.2 (2.70 g, 8.30 mmol), intermediate 1.2 (2.76 mL, 10.79 mmol), and TFA (0.63 mL, 0.83 mmol) in toluene (15 mL). After work up and chromatographic purification (PET/EtOAc, from 90:10 to 50:50, v/v), the second eluate is the diasteroisomer 23.3 (1.48 g, 3.24 mmol). Yield: 39%. MS-ESI(+) m/z 459.4 (M+H).

### Step 4: tert-Butyl (3R,4S)-3-[(4R)-benzyl-2-oxo-oxazolidine-3-carbonyl]-4-(4-fluorophenyl)-pyrrolidine-1-carboxylate (23.4)

Intermediate 23.4 was synthesized according to the procedure described in Step 2 of Example 1 from intermediate 23.3 (1.36 g, 3.05 mmol), DIPEA (0.58 mL, 3.35 mmol), and 1-chloroethylchloroformate (0.81 mL, 7.63 mmol) in CH₂Cl₂ (60 mL). The obtained crude was treated in refluxing MeOH (60 mL). After removal of volatiles, the debenzylated intermediate was reacted with Boc₂O (0.99 g, 4.57 mmol) and DIPEA (1.59 mL, 9.15 mmol) in CH₂Cl₂ (30 mL). After work-up the crude was purified by flash chromatography (PET/EtOAc, 90:10 to 60:40, v/v), to afford 1.42 g of intermediate 23.4. Yield: Quantitative. MS-ESI(+) m/z 469.4 (M+H).

### Step 5: (3R,4S)-1-(tert-Butoxycarbonyl)-4-(4-fluorophenyl)-pyrrolidine-3-carboxylic acid (23.5)

The intermediate 23.5 was synthesized according to the procedure reported in Step 4 of Example 17, from intermediate 23.4 (1.40 g, 2.98 mmol), 4.0 M aq. LiOH (2.98 mL, 11.95 mol), and 30% aq. H₂O₂ (4.56 mL, 44.70 mmol) in THF (50 mL) and H₂O (12 mL). After work-up the title intermediate 23.5 was obtained as a white powder (0.48 g, 1.55 mmol, yield 53%). MS-ESI(−) m/z 308.5 (M−H).

### Example 24: (3S,4R)-1-(tert-Butoxycarbonyl)-4-(4-fluorophenyl)-pyrrolidine-3-carboxylic acid (24.3)

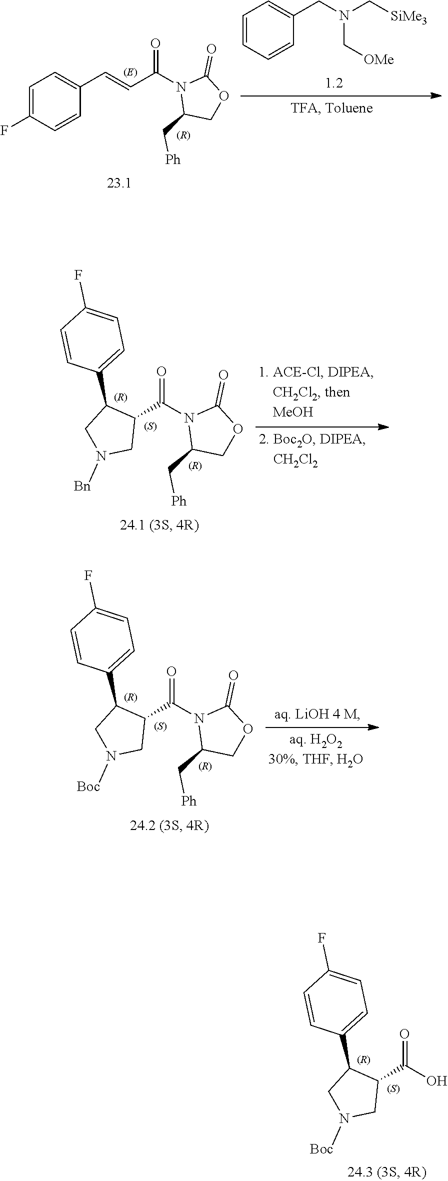

### Step 1: (4R)-Benzyl-3-[(3S,4R)1-benzyl-4-(4-fluorophenyl)-pyrrolidine-3-carbonyl]-oxazolidin-2-one (24.1)

The diasteroisomer 24.1 was synthesized according to the experimental procedure of Step 2 of Example 17, starting from intermediate 23.1 (2.70 g, 8.30 mmol), intermediate 1.2 (2.76 mL, 10.79 mmol), and TFA (0.063 mL, 0.83 mmol) in toluene (16 mL). After work up and chromatographic purification (PET/EtOAc, from 100% PET to 60:40 v/v PET/EtOAc), the first eluate is the diasteroisomer 24.1 obtained as a white solid (1.59 g, 3.48 mmol). Yield: 42%. MS-ESI(+) m/z 459.4 (M+H).

### Step 2: tert-Butyl (3S,4R)-3-[(4R)-benzyl-2-oxo-oxazolidine-3-carbonyl]-4-(4-fluorophenyl)-pyrrolidine-1-carboxylate (24.2)

Intermediate 24.2 was synthesized according to the procedure described in Step 2 of Example 1 from intermediate 24.1 (1.58 g, 3.44 mmol), DIPEA (0.66 mL, 3.78 mmol), and 1-chloroethylchloroformate (0.97 mL, 8.61 mmol) in CH₂Cl₂ (60 mL). The obtained crude was treated in refluxing MeOH (60 mL). After removal of volatiles, the debenzylated intermediate was reacted with Boc₂O (1.11 g, 5.10 mmol) and DIPEA (1.80 mL, 10.32 mmol) in CH₂Cl₂ (60 mL). After work-up, the crude was purified by flash chromatography (PET/EtOAc, from 100% PET to 70:30, v/v PET/EtOAc), to afford 1.60 g (3.43 mmol) of intermediate 24.2 as a pale yellow oil. Yield: 99%. MS-ESI(+) m/z 469.3 (M+H).

### Step 3: (3S,4R)-1-(tert-Butoxycarbonyl)-4-(4-fluorophenyl)-pyrrolidine-3-carboxylic acid (24.3)

The intermediate 24.3 was synthesized according to the procedure reported in Step 4 of Example 17, from 24.2 (1.40 g, 2.98 mmol), 4.0 M aq. LiOH (3.0 mL, 11.95 mmol), and 30% aq. H₂O₂ (4.56 mL, 44.7 mmol) in THF (50 mL) and H₂O (12 mL). After work-up, the title intermediate 24.3 was obtained as a white powder (0.79 g, 2.56 mmol, yield 86%). MS-ESI(−) m/z 308.6 (M−H).

### Example 25: (3R,4S)-1-(tert-Butoxycarbonyl)-4-(4-trifluoromethylphenyl)-pyrrolidine-3-carboxylic acid (25.5)

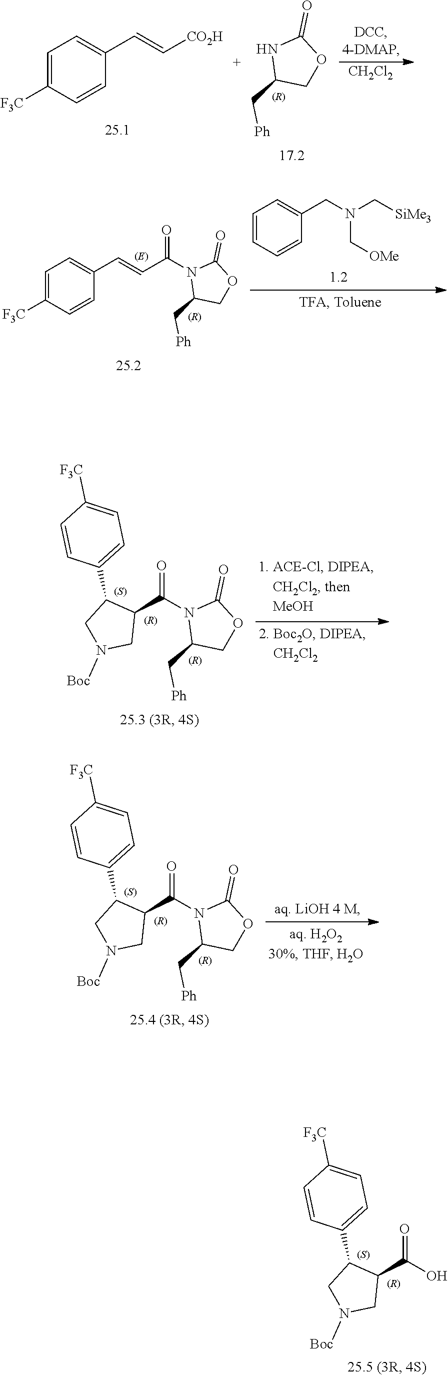

### Step 1: (4R)-Benzyl-3-[(3R,4S)1-benzyl-4-(4-trifluoromethylphenyl)-pyrrolidine-3-carbonyl]-oxazolidin-2-one (25.2)

The intermediate 25.2 was synthesized according to the experimental procedure of Step 1 of Example 17, starting from intermediate 25.1 (1.15 g, 5.32 mmol), intermediate 17.2 (0.94 g, 5.32 mmol), DMAP (85 mg, 0.69 mmol), and DCC (1.32 g, 6.38 mmol) in CH₂Cl₂ (20 mL). After work up and chromatographic purification (PET/EtOAc from 90:10 to 70:30, v/v), the intermediate 25.2 was obtained in nearly quantitative yield (2.00 g, 5.32 mmol). MS-ESI(+) m/z 376.5 (M+H).

### Step 2: (4R)-Benzyl-3-[(3R,4S)1-benzyl-4-(4-trifluoromethyphenyl)-pyrrolidine-3-carbonyl]-oxazolidin-2-one (25.3)

The diasteroisomer 25.3 was synthesized according to the experimental procedure of Step 2 of Example 17, starting from intermediate 25.2 (2.00 g, 5.32 mmol), intermediate 1.2 (1.63 mL, 6.38 mmol), and TFA (0.07 mL, 0.81 mmol) in toluene (20 mL). After work up and chromatographic purification (PET/EtOAc, from 80:20 to 40:60, v/v), the second eluate is the diasteroisomer 25.3 (0.57 g, 1.12 mmol). Yield: 21%. MS-ESI(+) m/z 509.4 (M+H).

### Step 3: tert-Butyl (3R,4S)-3-[(4R)-benzyl-2-oxo-oxazolidine-3-carbonyl]-4-(4-trifluoromethylphenyl)-pyrrolidine-1-carboxylate (25.4)

Intermediate 25.4 was synthesized according to the procedure described in Step 2 of Example 1 from intermediate 25.3 (700 mg, 1.59 mmol), DIPEA (0.30 mL, 1.75 mmol), and 1-chloroethylchloroformate (0.42 mL, 3.97 mmol) in CH₂Cl₂ (30 mL). The obtained crude was treated in refluxing MeOH (30 mL). After removal of volatiles, the debenzylated intermediate was reacted with Boc₂O (0.52 g, 2.38 mmol) and DIPEA (0.83 mL, 4.37 mmol) in CH₂Cl₂ (30 mL). After work-up the crude was purified by flash chromatography (PET/EtOAc, from 70:20 to 50:50, v/v), to provide the intermediate 25.4 in nearly quantitative yield (820 mg, 1.58 mmol). MS-ESI(+) m/z 519.4 (M+H).

### Step 4: (3R,4S)-1-(tert-Butoxycarbonyl)-4-(4-trifluoromethylphenyl)-pyrrolidine-3-carboxylic acid (25.5)

The intermediate 25.5 was synthesized according to the procedure reported in Step 4 of Example 17, from intermediate 25.4 (1.40 g, 2.70 mmol), 4.0 M aq. LiOH (2.7 mL, 10.79 mmol), and 30% aq. H₂O₂ (4.13 mL, 40.50 mol) in THF (50 mL) and H₂O (12 mL). After work-up, the title intermediate 25.5 was obtained as a white powder in 81% yield (0.79 g, 2.19 mmol). MS-ESI(−) m/z 358.6 (M−H).

### Example 26: (3S,4R)-1-(tert-Butoxycarbonyl)-4-(4-trifluorophenyl)-pyrrolidine-3-carboxylic acid (26.3)

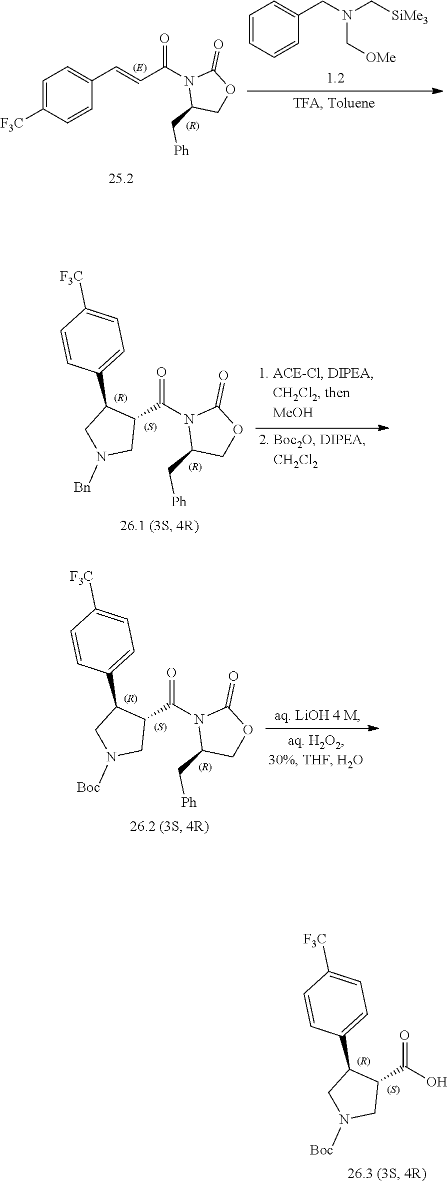

### Step 1: (4R)-Benzyl-3-[(3S,4R)1-benzyl-4-(4-trifluoromethyphenyl)-pyrrolidine-3-carbonyl]-oxazolidin-2-one (26.1)

The diasteroisomer 26.1 was synthesized according to the experimental procedure of Step 2 of Example 17, starting from intermediate 25.2 (2.00 g, 5.32 mmol), intermediate 1.2 (1.63 mL, 6.38 mmol), and TFA (0.07 mL, 0.81 mmol) in toluene (20 mL). After work up and chromatographic purification (PET/EtOAc, from 80:20 to 40:60, v/v), the first eluate is the diasteroisomer 26.1 (0.89 g, 1.75 mmol). Yield: 33%. MS-ESI(+) m/z 509.4 (M+H).

### Step 2: tert-Butyl (3S,4R)-3-[(4R)-benzyl-2-oxo-oxazolidine-3-carbonyl]-4-(4-trifluoromethylphenyl)-pyrrolidine-1-carboxylate (26.2)

Intermediate 26.2 was synthesized according to the procedure described in Step 2 of Example 1 from intermediate 26.1 (2.00 g, 3.93 mmol), DIPEA (0.75 mL, 4.32 mmol), and 1-chloroethylchloroformate (1.06 mL, 9.83 mmol) in CH₂Cl₂ (40 mL). The obtained crude was treated in refluxing MeOH (30 mL). After removal of volatiles, the debenzylated intermediate was reacted with Boc₂O (1.76 g, 7.86 mmol) and DIPEA (2.05 mL, 11.796 mmol) in CH₂Cl₂ (40 mL). After work-up, the crude of intermediate 26.2 was used such as for the next step. MS-ESI(+) m/z 519.4 (M+H).

### Step 3: (3S,4R)-1-(tert-Butoxycarbonyl)-4-(4-trifluoromethylphenyl)-pyrrolidine-3-carboxylic acid (26.3)

The intermediate 26.3 was synthesized according to the procedure reported in Step 4 of Example 17, from intermediate 26.2 (crude of previous step, 3.93 mmol), 4.0 M aq. LiOH (3.9 mL, 15.72 mol), and 30% aq. H₂O₂ (4.01 mL, 39.32 mol) in THF (35 mL) and H₂O (6 mL). After work-up, the title intermediate 26.3 was obtained as a white powder (1.09 g, 3.03 mmol, yield 77% from 6.1). MS-ESI(−) m/z 358.6 (M−H).

### Example 27: 5-Isothiocyanatoisoquinoline (27.2)

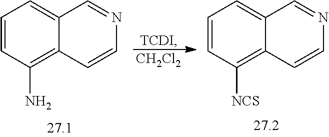

1,1′-thiocarbonyldiimidazole (3.78 g, 21.24 mmol) was added to a stirred solution of intermediate 27.1 (2.04 g) in CH₂Cl₂ (20 mL), and the reaction was stirred at r.t. for 24 h. The mixture was concentrated under reduced pressure and purified by flash chromatography (PET/EtOAc from 85:15 to 60:40 v/v). 2.01 g of the title intermediate 27.2 were obtained (76%).

MS-ESI(+) m/z: 187.3 (M+H).

### Example 28: 3-(2-Bromo-1,3-thiazol-4-yl)pyridine (28.4)

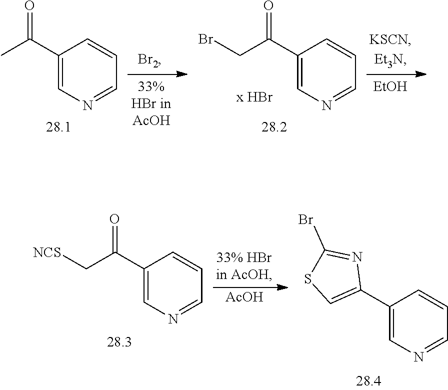

### Step 1: 2-Bromo-1-(pyridin-3-yl)ethanone hydrochloride (28.2)

Bromine (0.51 mL, 10.01 mmol) was added to a stirred solution of intermediate 28.1 (1.10 g, 9.09 mmol) in 33% HBr in AcOH (10 mL) cooled at 0° C. The mixture was then slowly warmed to 70° C. and reacted for 1 h. Once cooled to r.t., the obtained suspension was poured into Et₂O (50 mL), and the solid collected by filtration under vacuum, to afford 2.47 g of intermediate 28.2 (97%).

MS-ESI(+) m/z: 199.6 (M+H), 201.6 (M+H).

### Step 2: 2-Oxo-2-(pyridin-3-yl)ethyl thiocyanate (28.3)

Et₃N (1.23 mL, 8.79 mmol) was added to a stirred suspension of intermediate 28.2 (2.47 g, 8.79 mmol) in EtOH (40 mL), to give a solution, potassium thiocyanate (0.94 g, 9.671 mmol) was then added and the mixture was reacted at 85° C. for 1 h. Once cooled to r.t. it was poured into H₂O (50 mL) and brine (50 mL) then extracted with EtOAc (3×50 mL). The collected organic layers were washed with brine (50 mL), dried over Na₂SO₄, and concentrated under reduced pressure, to give 1.53 g of intermediate 28.3 which was used such as for the next step.

MS-ESI(+) m/z: 178.7 (M+H).

### Step 3: 3-(2-Bromo-1,3-thiazol-4-yl)pyridine (28.4)

The crude coming from the previous step (8.79 mmol) was dissolved in AcOH (7.5 mL) and treated with 33% HBr in AcOH (15 mL) at 50° C. for 16 h. Once cooled to r.t. the suspension was poured into Et₂O (50 mL) and filtered under vacuum. The collected solid was dissolved in H₂O (50 mL) and aq. NaHCO₃ ss was added up to pH=8.0. The mixture was extracted with EtOAc (3×50 mL), and the collected organic layers were washed with brine (50 mL), dried over Na₂SO₄, and concentrated under reduced pressure. After chromatographic purification (CH₂Cl₂/MeOH from 98:2 to 93:7 v/v), 1.09 g of title intermediate 28.4 were obtained as a pale yellow solid. Yield: 51%.

MS-ESI(+) m/z: 240.5 (M+H), 242.5 (M+H).

### Example 29: 2-Bromo[1,3]thiazolo[4,5-c]pyridine (29.5)

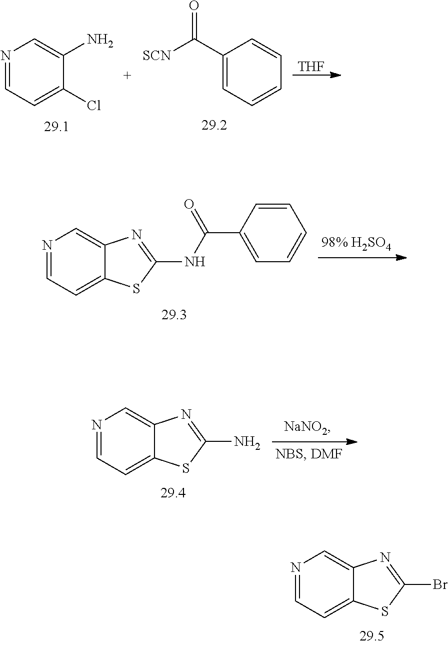

### Step 1: N-([1,3]Thiazolo[4,5-c]pyridin-2-yl)benzamide (29.3)

Intermediate 29.1 (500 mg, 3.89 mmol) was added to a stirred solution of intermediate 29.2 (0.73 mL, 5.44 mmol) in THF (15 mL) under N₂ atmosphere. The mixture thus obtained was reacted under magnetic stirring at 50° C. for 18 h. The mixture was cooled to r.t., the precipitate formed was filtered, washed with THF (3 mL), dried in drying oven under vacuum, to obtain 898 mg (3.52 mmol) of the desired intermediate 29.3, which was used such as for the next step. Yield: 90%. MS-ESI(+) m/z: 256.0 (M+H); MS-ESI(−) m/z: 253.9 (M−H).

### Step 2: [1,3]Thiazolo[4,5-c]pyridin-2-amine (29.4)

A solution of intermediate 29.3 in 98% H₂SO₄ was heated to 110° C. for 18 h under magnetic stirring. The solution was then cooled to r.t. and slowly poured into 6M aq. NaOH (30 mL) maintained at 0° C. The solid thus obtained was filtered off and washed with EtOAc (3×10 mL) and MeOH (3×5 mL). The phases were separated and the aqueous layer was extracted with EtOAc/MeOH (8:2, v/v). The combined organics were dried over anhydrous Na₂SO₄, and evaporated to dryness, to provide the desired crude intermediate 29.4, which was used such as for the next step. MS-ESI(+) m/z: 150.3 (M+H).

### Step 3: 2-Bromo[1,3]thiazolo[4,5-c]pyridine (29.5)

NaNO₂ (122 mg, 1.76 mmol) and N-bromosuccinimide (209 mg, 1.17 mmol) were added to a stirred solution of intermediate 29.4 (crude of previous step, 1.17 mmol) in DMF (5 mL), and the mixture was reacted for 3 h at r.t. The reaction was poured into H₂O (15 mL) and extracted with EtOAc (3×15 mL). The collected organic layers were washed with brine (25 mL), dried over Na₂SO₄, and concentrated under reduced pressure. After chromatographic purification (CH₂Cl₂/MeOH from 99:1 to 96:4 v/v), 80 mg (0.37 mmol) of the title intermediate 29.5 were obtained. Yield: 32%. MS-ESI(+) m/z: 214.9 (M+H), 217.0 (M+H).

### Example 30: 4-(Pyridin-3-yl)-1,3-thiazol-2-amine (30.1)

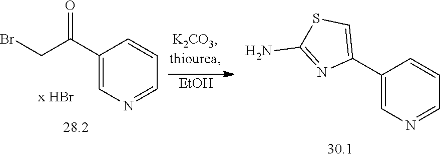 [SMILES: "EtOH" ; O=C(CBr)c1cccnc1 ; Nc1nc(-c2cccnc2)cs1]

Thiourea (247 mg, 3.24 mmol) and K₂CO₃ (814 mg, 5.90 mmol) were added to a stirring solution of intermediate 28.2 (830 mg, 2.95 mmol) in EtOH (15 mL), and the mixture thus obtained was reacted in refluxing conditions for 5 h. After cooling to r.t., the solvent was removed in vacuo, the residue was taken up with ssNaHCO₃ (50 mL) and stirring was continued at r.t. for 1 h. The aqueous mixture was extracted with EtOAc (3×50 mL), the organic layer was then dried over anhydrous Na₂SO₄, and evaporated to dryness to afford the title intermediate 30.1 (500 mg, 2.82 mmol) as a yellowish solid. Yield: 96%. MS-ESI(+) m/z: 176.6 (M+H).

### Example 31: 3-(3-Pyridyl)aniline (31.3)

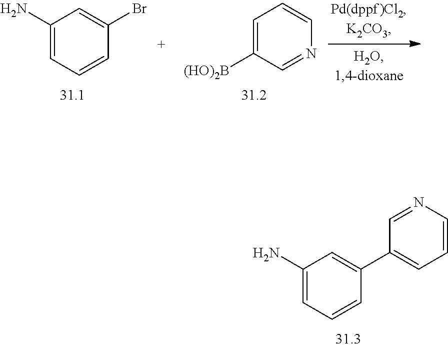 [SMILES: Nc1cccc(Br)c1 ; OB(O)c1cccnc1 ; Nc1cccc(-c2cccnc2)c1]

Intermediate 31.1 (2.32 g, 18.89 mmol) was dissolved in 1,4-dioxane (75 mL) and H₂O (25 mL) under N₂ atmosphere, then K₂CO₃ (8.02 g, 58.13 mmol), Pd(dppf)Cl₂ (532 mg, 0.73 mmol), and intermediate 31.2 (5.00 g, 14.53 mmol) were sequentially added. The mixture was reacted at 80° C. for 4 h. Thus, 1 M aq. NaOH (100 mL) was added and the reaction mixture was extracted with CH₂Cl₂ (3×50 mL). The combined organic layers were washed with H₂O (100 mL) and brine (100 mL), dried over anhydrous Na₂SO₄, and evaporated to dryness. After purification by flash chromatography (CH₂Cl₂/MeOH, from 100% CH₂Cl₂ to 95:5 v/v CH₂Cl₂/MeOH) the title intermediate 31.3 (2.02 g, 11.87 mmol) was obtained as a brown solid. Yield: 82%. MS-ESI(+) m/z: 171.4 (M+H).

### Example 32: 3-(6-Fluoropyridin-3-yl)aniline (32.3)

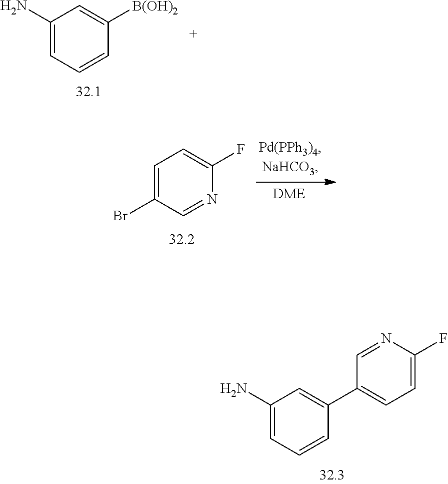 [SMILES: Nc1cccc(B(O)O)c1 ; Fc1ccc(Br)cn1 ; Nc1cccc(-c2ccc(F)nc2)c1]

To a stirred solution of intermediate 32.2 (0.38 mL, 3.65 mmol) in DME (15 mL), Pd(PPh₃)₄ (42 mg, 0.036 mmol) was added, after stirring for 10 minutes, intermediate 32.1 (500 mg, 3.65 mmol) and 1.0 M aq. NaHCO₃ (11 mL, 11.00 mmol) were then added sequentially. The mixture was reacted under refluxing conditions for 3 h. After cooling H₂O (100 mL) and EtOAc were added (50 mL), the phases were separated and the aqueous phase extracted with EtOAc (2×50 mL). The combined organic layers were washed with H₂O (50 mL) and brine (50 mL), dried over anhydrous Na₂SO₄, and evaporated to dryness. After work-up and chromatographic purification (PET/EtOAc from 95/5 to 75/25, v/v), the title intermediate 32.3 (618 mg, 3.28 mmol) was obtained in 90% yield. MS-ESI(+) m/z: 189.4 (M+H).

### Example 33: 3-Isothiocyanatothieno[2,3-c]pyridine (33.5)

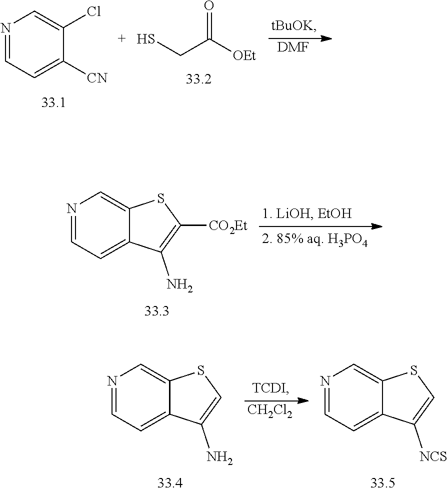

### Step 1: Ethyl 3-aminothieno[2,3-c]pyridine-2-carboxylate (33.3)

To a stirred solution of intermediate 33.1 (1.5 g, 10.83 mmol) in DMF (9 mL) cooled to 0° C., intermediate 33.2 (1.19 mL, 10.83 mmol) and tBuOK (1.21 g, 10.83 mmol) were sequentially added and the resulting solution was reacted at 0° C. for 30 min, then at r.t. for 16 h. The mixture was slowly poured into H₂O (40 mL) maintained under magnetic stirring, the brownish solid thus obtained was collected by filtration and used such as for further processing.

MS-ESI(+) m/z: 220.8 (M+H); MS-ESI(−) m/z: 222.9 (M−H).

### Step 2: Thieno[2,3-c]pyridin-3-amine (33.4)

LiOH (216.mg, 9.00 mmol) was added to a stirred solution of 33.3 (crude of previous step, ca. 500 mg, 2.25 mmol) in EtOH (15 mL), and the mixture was stirred under reflux for 16 h. The suspension was cooled to r.t., then 1 N aq. HCl (5.5 mL) and H₂O (50 mL) were added (pH ˜ 6), and the yellow precipitate thus obtained was collected by filtration. The resulting solid was dissolved in 85% aq. H₃PO₄ (3.6 mL) and stirred at 60° C. for 16 h. The solution was cooled to r.t. and slowly poured into a 5 M aq. solution of NaOH (18.6 mL) maintained at 0° C. under magnetic stirring. The precipitate was filtered and washed with EtOAc (20 mL). The phases were separated, and the aqueous phase was extracted with EtOAc (2×10 mL). the combined organic layers were washed with brine (20 mL), dried over Na₂SO₄, and evaporated to dryness. The crude intermediate 33.4 was used directly for the next step.

MS-ESI(+) m/z: 150.9 (M+H); MS-ESI(−) m/z: 149.7 (M−H)

### Step 3: 3-Isothiocyanatothieno[2,3-c]pyridine (33.5)

The intermediate 33.5 was synthesized according to the procedure reported in Example 27 from intermediate 33.4 (crude of previous step, 2.25 mmol) and TCDI (0.60 g, 3.37 mmol) in CH₂Cl₂ (20 mL). After chromatographic purification (PET/EtOAc), 21.5 g of the title intermediate 33.5 was obtained in 27% yield from 33.1.

MS-ESI(+) m/z: 193.4 (M+H).

### Example 34: (2E)-3-Phenyl-N-[3-(pyridin-3-yl)phenyl]prop-2-enamide (34.1)

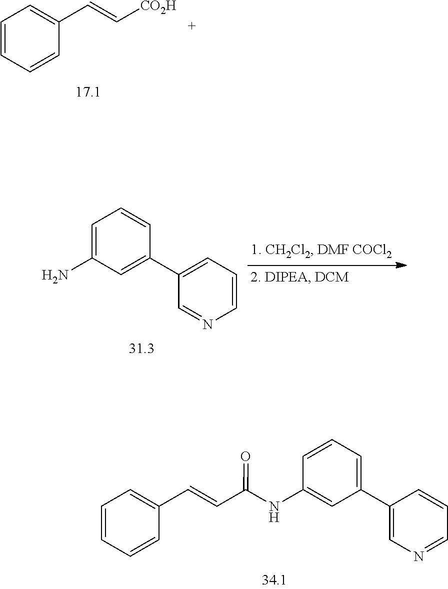 [SMILES: O=C(O)/C=C/c1ccccc1 ; Nc1cccc(-c2cccnc2)c1 ; O=C(/C=C/c1ccccc1)Nc1cccc(-c2cccnc2)c1]

Oxalyl chloride (0.27 mL, 3.10 mmol) was added to a stirred solution of intermediate 17.1 (300 mg, 2.03 mmol) in CH₂Cl₂ (10 mL) and DMF (2 drops) cooled at 0-5° C., and the mixture was reacted at r.t. for 4 h. The volatiles were removed under reduced pressure, the crude was dissolved in CH₂Cl₂ (15 mL) and to the resulting solution were added intermediate 31.3 (390 mg, 2.23 mmol) and DIPEA (0.39 mL, 2.23 mmol). The mixture was stirred for 16 h, then washed with aq. citric acid 0.5 M (3×20 mL), brine (20 mL), dried over Na₂SO₄, and concentrated under reduced pressure. After chromatographic purification, the title intermediate 34.1 was obtained in 74% yield.

MS-ESI(+) m/z: 301.4 (M+H).

### Example 35: N-Methyl-3-(pyridin-3-yl)aniline (35.2)

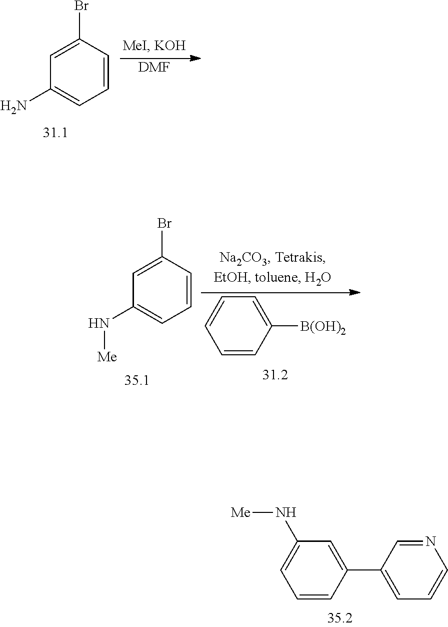

### Step 1: 3-Bromo-N-methylaniline (35.1)

KOH (179 mg, 3.20 mmol) and MeI (0.18 mL, 2.91 mmol) were sequentially added to a stirred solution of intermediate 31.1 (0.32 mL, 2.91 mmol) in DMF (3 mL) and magnetic stirring was continued for 3 days at r.t. The mixture was poured into H₂O (50 mL) and extracted with EtOAc (3×20 mL). The combined organic layers were washed with H₂O (50 mL) and brine (50 mL), dried over anhydrous Na₂S04, and evaporated to dryness. After purification by flash chromatography (PET/EtOAc, from 100% PET to 8:2 v/v PET/EtOAc), the title intermediate 35.1 (275 mg, 1.48 mmol) was obtained. Yield: 91%. MS-ESI(+) m/z: 185.9, 187.9 (M+H).

### Step 2: N-Methyl-3-(pyridin-3-yl)aniline (35.2)

To a degassed solution of intermediate 35.1 (270 mg, 1.45 mmol) in EtOH/toluene (1:1 v/v, 10 mL), intermediate 30.2 (193 mg, 1.57 mmol) and a solution of Na₂CO₃ (900 mg, 8.49 mmol) in H₂O (4 mL) were sequentially added and the mixture was reacted at 80° C. for 21 h. The mixture was extracted with EtOAc (3×20 mL). The combined organic layers were washed with H₂O (30 mL) and brine (30 mL), dried over anhydrous Na₂S04, and evaporated to dryness. After purification by flash chromatography (PET/EtOAc, from 100% PET to 6:4 v/v PET/EtOAc) the title intermediate 35.2 (156 mg, 0.85 mmol) was obtained as a pale yellow oil. Yield: 59%. MS-ESI(+) m/z: 185.0 (M+H).

### Example 36: 3-(Azetidin-3-yl)pyridine (36.4)

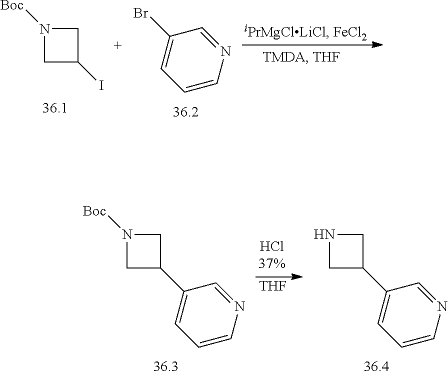 [SMILES: CC(C)(C)OC(=O)N1CC(I)C1 ; Brc1cccnc1 ; "THF" ; CC(C)(C)OC(=O)N1CC(c2cccnc2)C1 ; c1cncc(C2CNC2)c1]

### Step 1: tert-Butyl 3-(pyridin-3-yl)azetidine-1-carboxylate (36.3)

ⁱPrMgCl.LiCl 1.3 M in THF (9.7 mL, 12.61 mmol) was added dropwise to a stirred solution of intermediate 36.2 (2.00 g, 12.61 mmol) in dry THF (12 mL) under N₂ atmosphere, and the resulting mixture was stirred at r.t. for 2 h. In meantime, in a second three neck round flask equipped with a dropping funnel, a solution of intermediate 36.1 (1.09 mL, 6.29 mmol), iron(II)chloride (80 mg, 0.63 mol) and tetramethylethylenediamine (0.09 mL, 0.63 mmol) in THF (40 mL) was prepared and cooled at 0-5° C. The contents of the first flask were then slowly added dropwise to the second one, maintaining the internal temperature below 5° C. Once the addition was complete, the mixture was vigorously stirred at r.t. for 3 h and then filtered through a celite pad under vacuum, washing the residual solid with EtOAc (30 mL). The collected organic liquor was washed with H₂O (50 mL), brine (50 mL), dried over Na₂SO₄, and concentrated under reduced pressure. After chromatographic purification (CH₂Cl₂/MeOH from 98:2 to 94:6, v/v), 1.24 g of intermediate 36.3 were obtained.

Yield: 42%

MS-ESI(+) m/z: 235.2 (M+H).

### Step 2: 3-(Azetidin-3-yl)pyridine (36.4)

A stirred solution of intermediate 36.3 (0.50 g, 2.13 mol) in THF (20 mL) was treated with aq. HCl 37% (0.61 mL, 8.52 mmol) at 40° C. for 2 h. The resulting solution was basified to pH=8.0 by adding aq. NaHCO₃ ss and the volatiles were removed under reduced pressure. The crude was purified by reverse phase flash chromatography (stationary phase: RP-18, elution with H₂O/MeOH from 80:20 to 25:75, v/v), to give the title intermediate 36.4 as a white powder in 77% yield.

MS-ESI(+) m/z: 176.2 (M+H+MeCN).

### Example 37: 3-(Pyridin-3-yloxy)aniline (37.4)

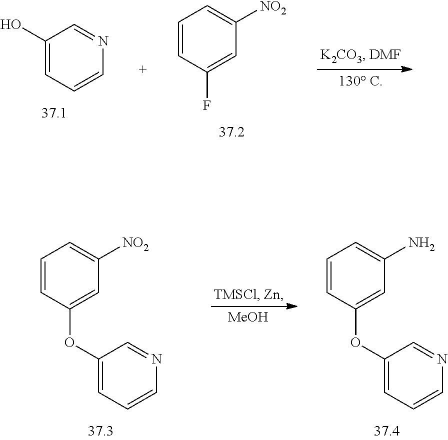 [SMILES: "MeOH" ; Oc1cccnc1 ; O=[N+]([O-])c1cccc(F)c1 ; O=[N+]([O-])c1cccc(Oc2cccnc2)c1 ; Nc1cccc(Oc2cccnc2)c1]

### Step 1: 3-(3-Nitrophenoxy)pyridine (37.3)

K₂CO₃ (1.96 g, 14.17 mmol) and intermediate 37.2 (0.76 mL, 7.09 mmol) were sequentially added to a stirred solution of intermediate 37.1 (674 mg, 7.09 mmol) in dry DMF (10 mL) maintained under N₂ atmosphere, and stirring was continued at 130° C. for 48 h. The mixture was poured into H₂O (150 mL) and extracted with CH₂Cl₂ (3×30 mL). The combined organic layers were washed with H₂O (80 mL) and brine (500 mL), dried over anhydrous Na₂SO₄, and evaporated to dryness. After purification by flash chromatography (PET/EtOAc, from 100% PET to 1:1 v/v PET/EtOAc) the desired intermediate 37.3 (805 mg, 3.72 mmol) was obtained as a brown oil. Yield: 53%. MS-ESI(+) m/z: 217.0 (M+H).

### Step 2: 3-(Pyridin-3-yloxy)aniline (37.4)

12N HCl (3.08 mL, 37.00 mmol), and zinc (726 mg, 11.10 mmol) were sequentially added to a solution of intermediate 37.3 (800 mg, 3.70 mmol) in MeOH (30 mL), and stirring was continued for 1 h at r.t. The catalyst was filtered over a celite pad, the liquor was diluted with H₂O (50 mL) and washed with EtOAc (2×20 mL). The aqueous layer was treated with aq. ss NaHCO₃ up to pH=9 and extracted with EtOAc (3×50 mL). The combined organic layers were washed with H₂O (50 mL) and brine (50 mL), dried over anhydrous Na₂SO₄, and evaporated to dryness. The title intermediate 37.4 (579 mg, 3.11 mmol) was obtained as a yellow solid. Yield: 84%. MS-ESI(+) m/z: 187.1 (M+H).

### Example 38: 3-(Pyridin-3-yloxy)aniline (38.2)

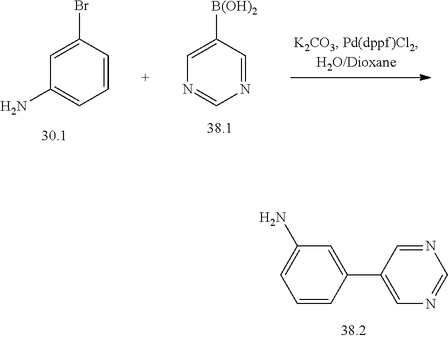 [SMILES: Nc1cccc(Br)c1 ; OB(O)c1cncnc1 ; Nc1cccc(-c2cncnc2)c1]

Intermediate 38.2 was prepared according to the procedure described in Example 100 starting from intermediate 30.1 (0.31 mL, 2.91 mmol), intermediate 38.1 (468 mg, 3.78 mmol), K₂CO₃ (1.60 g, 11.63 mmol), and Pd(dppf)Cl₂ (106 mg, 0.15 mmol) in H₂O (4 mL) and 1,4-dioxane (12 mL). Stirring was continued at 80° C. for 4 h. After workup and purification by flash chromatography (CH₂Cl₂/MeOH, from 100% CH₂Cl₂ to 95:5 v/v CH₂Cl₂/MeOH), the title intermediate 38.2 (446 mg, 2.61 mmol) was obtained as a white crystalline solid. Yield: 90%. MS-ESI(+) m/z: 172.1 (M+H).

### Example 39: N-Methylisoquinolin-5-amine (39.1)

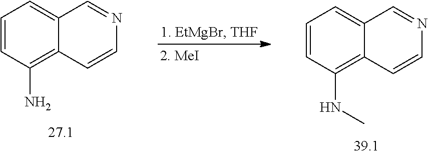

3.0 M EtMgBr in Et₂O (1.16 mL, 3.47 mmol) was added dropwise to a solution of intermediate 27.1 (500 mg, 3.47 mmol) in THF (8 mL) maintained under N₂ atmosphere. After 5 minutes, MeI (195 mL, 3.12 mmol) was added dropwise and the resulting fine suspension was reacted at r.t. for 3 h. The mixture was poured into H₂O (30 mL) and extracted with EtOAc (3×10 mL). The combined organic layers were washed with 0.5 M aq. citric acid (30 mL), H₂O (30 mL), and brine (30 mL), dried over anhydrous Na₂SO₄, and evaporated to dryness. After purification by flash chromatography (DCM/MeOH, from 98:2 v/v to 94:6 v/v) the title intermediate 39.1 (265 mg, 1.66 mmol) was obtained. Yield: 48%. MS-ESI(+) m/z: 159.2 (M+H).

### Example 40: 4-(Pyridin-3-yl)aniline (40.2)

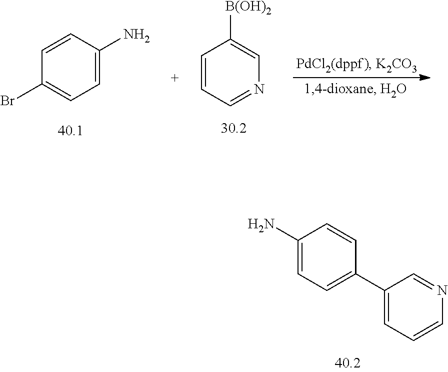 [SMILES: Nc1ccc(Br)cc1 ; OB(O)c1cccnc1 ; Nc1ccc(-c2cccnc2)cc1]

K₂CO₃ (3.24 g, 23.48 mmol) and [1,1′-bis(diphenylphosphino)ferrocene]palladium(II) dichloride (0.22 g, 0.29 mmol) were added to a stirred solution of intermediate 40.1 (1.00 g, 5.81 mmol) and intermediate 30.2 (0.94 g, 7.64 mmol) in 1,4-dioxane (30 mL), and H₂O (12 mL). The resulting mixture was reacted at 110° C. for 16 h. Once cooled to r.t. the reaction was diluted with EtOAc (30 mL) and filtered through a celite pad under vacuum. Collecting the liquor, the two phases were separated and the aqueous phase extracted with EtOAc (2×15 mL). The collected organic layers were washed with brine (30 mL), dried over Na₂SO₄, and concentrated under reduced pressure. After chromatographic purification (CH₂Cl₂/MeOH from 99:1 to 95:5), 0.70 g of the title intermediate 40.2 were obtained. Yield: 71%

MS-ESI(+) m/z: 171.3 (M+H).

### Example 41: l-Methylisoquinolin-5-amine (41.3)

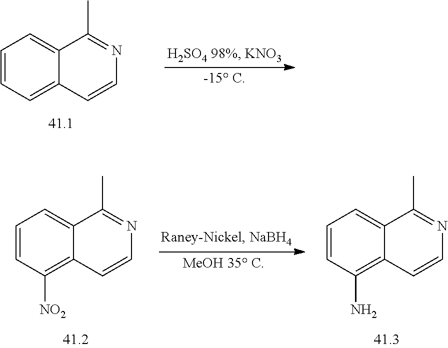

### Step 1: 1-Methyl-5-nitroisoquinoline (41.2)

A solution of KNO₃ (353 mg, 3.49 mmol) in H₂SO₄ 98% (2 mL) was added dropwise to a stirred solution of intermediate 41.1 (500 mg, 3.49 mmol) in H₂SO₄ 98% (2 mL) cooled at −15° C. The mixture was allowed to stir at r.t. and reacted for 3 h. The mixture was then cautiously poured into 5.0 M aq. NaOH (20 mL) and extracted with CH₂Cl₂ (3×20 mL). The collected organic layers were dried over Na₂SO₄ and concentrated under reduced pressure, to afford 650 mg of intermediate 41.2 as a pale yellow powder. Yield: quantitative.

MS-ESI(+) m/z: 189.1 (M+H).

### Step 2: 1-Methylisoquinolin-5-amine (41.3)

Aq. Raney-Nickel suspension (1.5 mL) was added to a stirred solution of intermediate 41.2 (650 mg, 3.45 mmol) in MeOH (10 mL), and the reaction was warmed at 35° C. Sodium borohydride (262 mg, 6.91 mmol) was then added portion wise. After 5 min the reaction was filtered through a celite pad under vacuum, washing the solid with CH₂Cl₂ (30 mL). The liquor was concentrated under reduced pressure, the crude was dissolved in CH₂Cl₂ (20 mL) then washed with brine (20 mL), dried over Na₂SO₄, and concentrated under reduced pressure, to give 505 mg of title intermediate 41.3. Yield: 92%

MS-ESI(+) m/z: 159.3 (M+H).

### Example 42: 1-Chloroisoquinolin-5-amine (42.3)

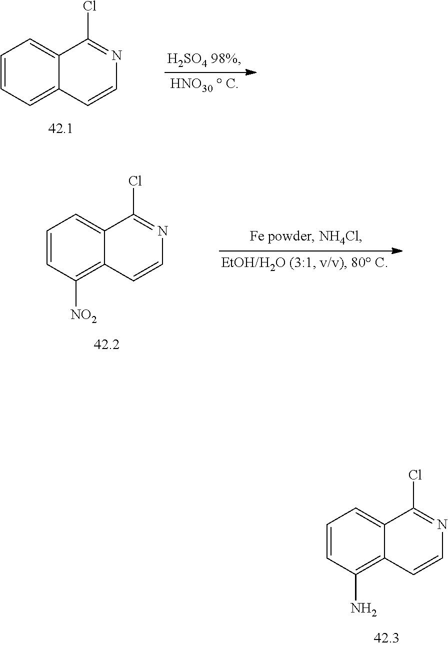 [SMILES: Clc1nccc2ccccc12 ; O=[N+]([O-])c1cccc2c(Cl)nccc12 ; Nc1cccc2c(Cl)nccc12]

### Step 1: 1-Chloro-5-nitroisoquinoline (42.2)

To a stirred solution of intermediate 42.1 (500 mg, 3.06 mmol) in 98% H₂SO₄ (2.3 mL) cooled to 0° C., was added fuming HNO₃ (0.44 mL, 10.70 mmol) and the resulting solution was reacted at r.t. for 3 h. The solution was then slowly poured into 6 M aq. NaOH cooled to 0° C., and the solid thus obtained was collected by filtration and dried under vacuum. The product was used such as without further purification. MS-ESI(+) m/z: 209.4 (M+H).

### Step 2: 1-Chloroisoquinolin-5-amine (42.3)

To a stirred suspension of intermediate 42.2 (200 mg, 0.96 mmol) in EtOH/H₂O (3:1 v/v, 6 mL), powdered iron (289 mg, 5.18 mmol), and NH₄Cl (31 mg, 0.58 mmol) were sequentially added. The mixture was reacted at 80° C. for 1 h. After cooling down to r.t., the solvent was removed in vacuo and the residue was submitted to chromatographic purification (CH₂Cl₂/MeOH from 98:2 to 90:10 v/v), to afford the title intermediate 42.3 (152 mg, 0.85 mmol) was obtained as a brownish solid. Yield: 89%. MS-ESI(+) m/z: 179.2 (M+H).

### Example 43: 5-Amino-2-methylisoquinolin-1(2H)-one (43.3)

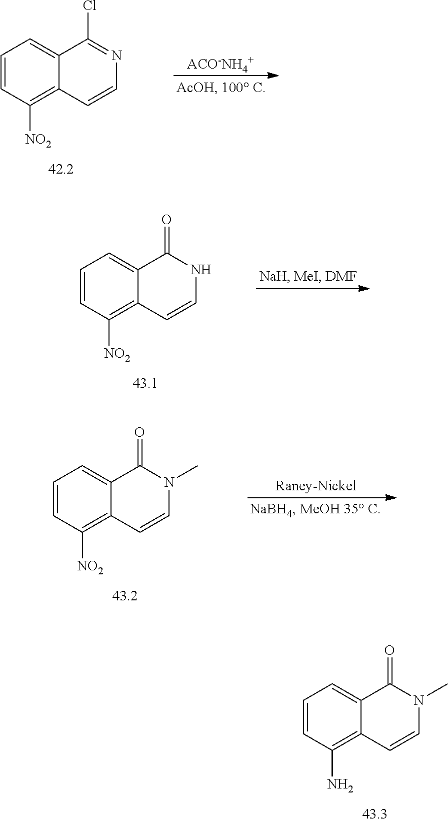

### Step 1: 5-Nitroisoquinolin-1(2H)-one (43.1)

A mixture of intermediate 42.2 (1.00 g, 4.79 mmol) and ammonium acetate (3.69 g, 47.94 mmol) in AcOH (10 mL) was stirred at 100° C. for 3 h. Once cooled to r.t., the reaction was poured into H₂O/ice (120 mL). The yellow precipitate thus obtained was filtered under vacuum, washing the solid with H₂O (2×10 mL). The collected solid was co-evaporated with acetone under reduced pressure, to give 0.61 g of intermediate 43.1.

Yield: 67%

MS-ESI(−) m/z: 189.1 (M−H).

### Step 2: 2-Methyl-5-nitroisoquinolin-1(2H)-one (43.2)

Intermediate 43.1 (500 mg, 2.63 mmol) was added to a stirred suspension of NaH 60% in mineral oil (420 mg, 10.517 mmol) in DMF (5 mL). After 5 min, methyl iodide (0.21 mL, 3.42 mmol) was added, and the mixture was stirred at r.t. for 2 h. The suspension thus obtained was diluted with EtOAc (50 mL), washed with 0.5 M citric acid (30 mL), brine (50 mL), dried over Na₂SO₄, and concentrated under reduced pressure. After chromatographic purification (CH₂Cl₂/MeOH from 98.5:1.5 to 94.6 v/v), 520 mg of the intermediate 43.2 were obtained.

Yield: 97%

MS-ESI(−) m/z: 203.3 (M−H).

### Step 3: 5-Amino-2-methylisoquinolin-1(2H)-one (43.3)

The intermediate 43.3 was synthesized according to the procedure reported in Step 2 of Example 41 from intermediate 48.2 (570 mg, 2.79 mmol), aq. Raney-Nickel suspension (2 mL), and sodium borohydride (212 mg, 5.58 mmol). After work up, 379 mg of the title intermediate 43.3 were obtained.

Yield: 78%

MS-ESI(+) m/z: 175.2 (M+H).

### Example 44: Isoquinolin-5-ylmethyl methanesulfonate (44.3)

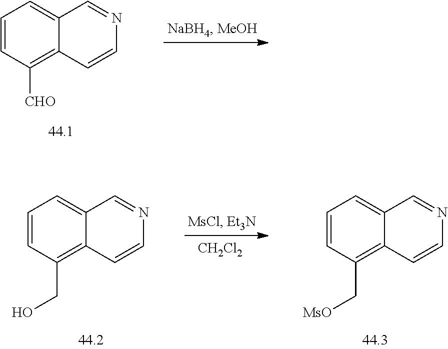

### Step 1: Isoquinolin-5-ylmethanol (44.2)

To a solution of intermediate 44.1 (300 mg, 1.91 mmol) in MeOH (8 mL) cooled to 0-5° C., sodium borohydride (87 mg, 2.29 mmol) was added, and the mixture was stirred for 2 h. The reaction was then poured into EtOAc (30 mL), washed with brine (20 mL), dried over Na₂SO₄, and concentrated under reduced pressure, to afford 285 mg of the intermediate 44.2.

Yield: 94%

MS-ESI(+) m/z: 160.3 (M+H).

### Step 2: Isoquinolin-5-ylmethyl methanesulfonate (44.3)

Mesyl chloride (0.20 mL, 2.64 mmol) and Et₃N (0.74 mL, 5.28 mmol) were added to a stirred solution of intermediate 44.2 (280 mg, 1.76 mmol) in CH₂Cl₂ (10 mL), and the resulting mixture was stirred at r.t. for 16 h. The reaction was poured into H₂O (15 mL), the two phases were separated and the aqueous phase extracted with CH₂Cl₂ (2×15 mL). The collected organic layers were washed with brine (20 mL), dried over Na₂SO₄, and concentrated under reduced pressure. After chromatographic purification (CH₂Cl₂/MeOH from 99:1 to 95:5, v/v), 117 mg of title intermediate 44.3 were obtained.

Yield: 28%.

MS-ESI(+) m/z: 178.3 (M+H−MsO+MeCN).

### Example 45: N-Phenylbenzene-1,3-diamine (45.5)

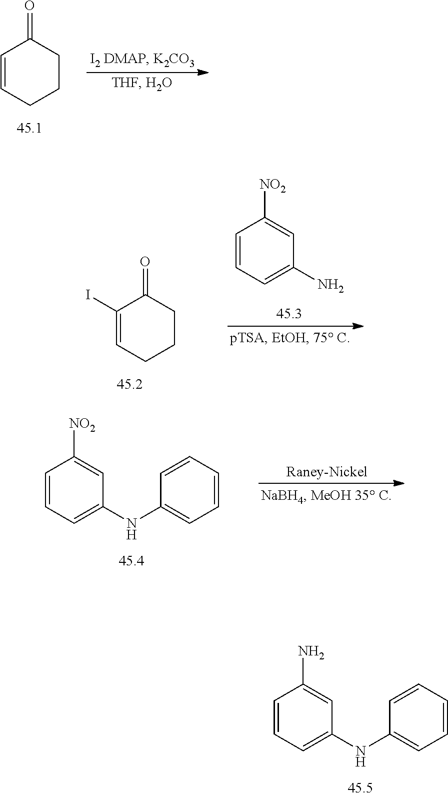 [SMILES: O=C1C=CCCC1 ; O=C1CCCC=C1I ; Nc1cccc([N+](=O)[O-])c1 ; O=[N+]([O-])c1cccc(Nc2ccccc2)c1 ; Nc1cccc(Nc2ccccc2)c1]

### Step 1: 2-Iodocyclohex-2-en-1-one (45.2)

A mixture of intermediate 45.1 (1.50 mL, 15.76 mmol), iodine (6.00 g, 23.64 mmol), DMAP (1.92 g, 15.76 mmol), and K₂CO₃ (2.61 g, 18.91 mmol) in THF/H₂O (50 mL, 1:1 v/v) was vigorously stirred at r.t. for 45 min. The obtained dark mixture was poured into EtOAc (50 mL), washed with aq. Na₂S₂O₃ ss (2×50 mL), brine (50 mL), dried over Na₂SO₄, and concentrated under reduced pressure. After chromatographic purification (PET/EtOAc from 98:2 to 90:10 v/v), 0.59 g of intermediate 45.2 were obtained.

Yield: 17%.

### Step 2: 3-Nitro-N-phenylaniline (45.4)

pTSA (0.15 g, 0.80 mmol) was added to a stirred solution of intermediate 45.2 (0.59 g, 2.68 mmol) and intermediate 45.3 (0.37 g, 2.68 mmol) in EtOH (4 mL), and the reaction was stirred at 75° C. for 90 min. Once cooled to r.t., the mixture was poured into EtOAc (25 mL), washed with aq. NaHCO₃ ss (20 mL), brine (20 mL), dried over Na₂SO₄, and concentrated under reduced pressure, to give 145 mg of intermediate 45.4 which was used such as for the next step.

Yield: 25%.

MS-ESI(+) m/z: 215.4 (M+H).

### Step 3: N-Phenylbenzene-1,3-diamine (45.5)

The intermediate 45.5 was synthesized according to the procedure reported in Step 2 of Example 41 from intermediate 45.4 (491 mg, 2.29 mmol), aq. Raney-Nickel suspension (1.03 mL), and sodium borohydride (173 mg, 4.58 mmol). After work up and purification by flash chromatography (PET/EtOAc, from 9:1 to 1:1 v/v) the title intermediate 45.5 (350 mg, 1.90 mmol) was obtained as a yellow solid. Yield: 83%. MS-ESI(+) m/z: 185.1 (M+H).

### Example 46: tert-Butyl (3-aminophenyl)phenylcarbamate (46.2)

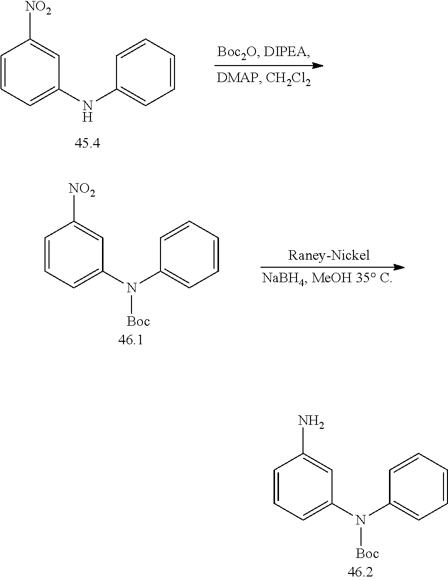 [SMILES: O=[N+]([O-])c1cccc(Nc2ccccc2)c1 ; CC(C)(C)OC(=O)N(c1ccccc1)c1cccc([N+](=O)[O-])c1 ; CC(C)(C)OC(=O)N(c1ccccc1)c1cccc(N)c1]

### Step 1: tert-Butyl (3-nitrophenyl)phenylcarbamate (46.1)

DMAP (16 mg, 0.13 mmol), and DIPEA (0.23 mL, 1.31 mmol) were added to a stirred solution of intermediate 45.4 (140 mg, 0.653 mmol) in CH₂Cl₂ (10 mL), Boc₂O (214 mg, 0.98 mmol). After 36 h the reaction was poured into H₂O (10 mL), the two phases were separated and the aqueous phase extracted with CH₂Cl₂ (2×5 mL). The collected organic layers were washed with aq. citric acid 0.5 M (15 mL), brine (15 mL), dried over Na₂SO₄, and concentrated under reduced pressure. After chromatographic purification (PET/EtOAc from 90:10 to 60:40 v/v), 210 mg of intermediate 46.1 were obtained.

Yield: 99%.

MS-ESI(+) m/z: 215.3 (M+H-100).

### Step 2: tert-Butyl (3-aminophenyl)phenylcarbamate (46.2)

The intermediate 46.2 was synthesized according to the procedure reported in Step 2 of Example 41 from intermediate 46.1 (210 mg, 0.66 mmol), aq. Raney-Nickel suspension (0.3 mL), and sodium borohydride (51 mg, 1.37 mmol) in MeOH (5 mL). After work up 172 mg of the title intermediate 46.2 were obtained.

Yield: 91%.

MS-ESI(+) m/z: 285.3 (M+H), 185.3 (M+H-100).

### Example 47: 3-(Tetrahydro-2H-pyran-4-yloxy)aniline (47.4)

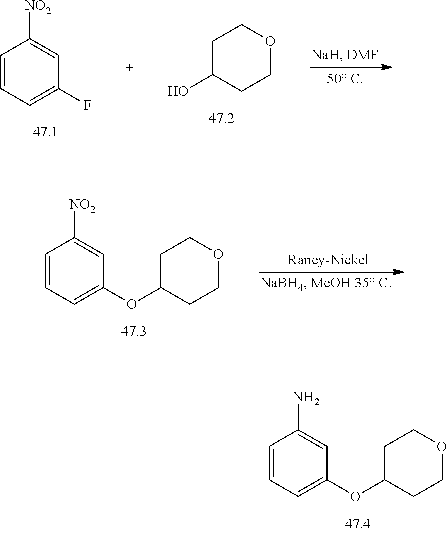 [SMILES: O=[N+]([O-])c1cccc(F)c1 ; OC1CCOCC1 ; O=[N+]([O-])c1cccc(OC2CCOCC2)c1 ; Nc1cccc(OC2CCOCC2)c1]

### Step 1: 4-(3-Nitrophenoxy)tetrahydro-2H-pyran (47.3)

A mixture of intermediate 47.1 (1.00 mL, 9.39 mmol) and intermediate 47.2 (1.16 mL, 12.20 mmol) in DMF (10 mL) was treated with NaH 60% in mineral oil (0.75 g, 18.78 mmol) at 50° C. for 5 h. Once cooled to r.t. the reaction was poured into EtOAc (30 mL), washed with H₂O (30 mL), brine (20 mL), dried over Na₂SO₄, and concentrated under reduced pressure. After chromatographic purification (PET/EtOAc from 90:10 to 65:35, v/v), 545 mg of intermediate 47.3 were obtained. Yield: 26%

MS-ESI(−) m/z: 222.1 (M−H)

### Step 2: 3-(Tetrahydro-2H-pyran-4-yloxy)aniline (47.4)

The intermediate 47.4 was synthesized according to the procedure reported in Step 2 of Example 41 from intermediate 47.3 (540 mg, 2.42 mmol), aq. Raney-Nickel suspension (0.5 mL), and sodium borohydride (184 mg, 4.84 mmol) in MeOH (7 mL). After work up 332 mg of the title intermediate 47.4 were obtained.

Yield: 72%.

MS-ESI(+) m/z: 285.3 (M+H).

### Example 48: 3-[4-(Trifluoromethyl)phenoxy]aniline (48.3)

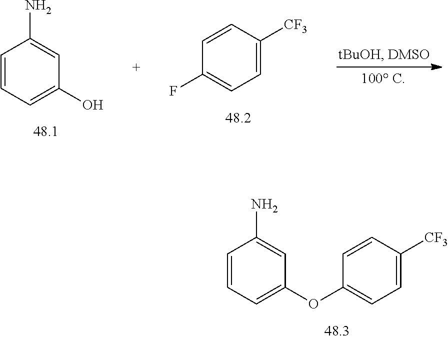 [SMILES: Nc1cccc(O)c1 ; Fc1ccc(C(F)(F)F)cc1 ; Nc1cccc(Oc2ccc(C(F)(F)F)cc2)c1]

A stirred solution of intermediate 48.1 (100 mg, 0.91 mmol), intermediate 48.2 (150 mg, 0.91 mmol), and ^{t}BuOK (118 mg, 1.05 mmol in DMSO (2 mL) was stirred at 100° C. for 16 h. Once cooled to r.t., the reaction was poured into EtOAc (15 mL), washed with LEO (2×10 mL), brine (10 mL), dried over Na₂SO₄, and concentrated under reduced pressure. After chromatographic purification (PET/EtOAc from 90:10 to 65:35 v/v), the title intermediate 48.3 was obtained in 77% yield as vitreous pale yellow solid.

MS-ESI(+) m/z: 254.1 (M+H).

### Example 49: 3-(4-Fluorophenoxy)aniline (49.2)

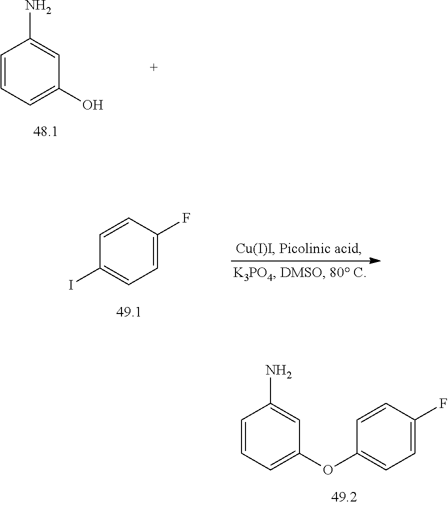 [SMILES: Nc1cccc(O)c1 ; Fc1ccc(I)cc1 ; Nc1cccc(Oc2ccc(F)cc2)c1]

Intermediate 48.1 (250 mg, 2.29 mmol), intermediate 49.1 (0.22 mL, 1.91 mmol), Cu(I)I (18 mg, 0.09 mmol), K₃PO₄ (767 mg, 3.82 mmol), and picolinic acid (24 mg, 0.19 mmol) were inserted in a tube, which was back-filled with N2 (3 times). DMSO (5 mL) was then added, the tube was sealed, and the resulting mixture was stirred at 80° C. for 24 h. Once cooled to r.t., the reaction was poured into EtOAc (25 mL), washed with H₂O (2×20 mL), brine (20 mL), dried over Na₂SO₄, and concentrated under reduced pressure. After chromatographic purification (PET/EtOAc from 95:5 to 70:30, v/v), the title intermediate 49.2 was obtained in 77% yield as whitish solid.

MS-ESI(+) m/z: 244.5 (M+H+MeCN).

### Example 50: 4-(3-Aminophenoxy)benzonitrile (50.2)

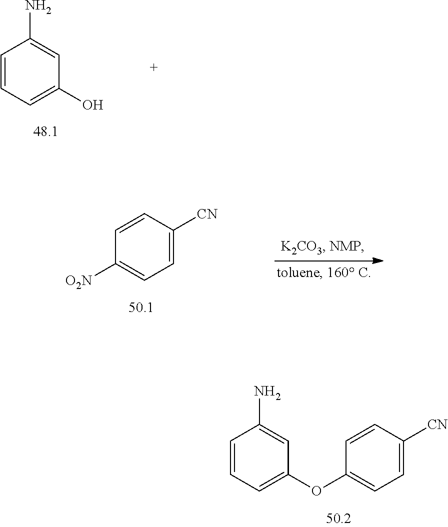 [SMILES: Nc1cccc(O)c1 ; N#Cc1ccc([N+](=O)[O-])cc1 ; N#Cc1ccc(Oc2cccc(N)c2)cc1]

A mixture of intermediate 48.1 (1.00 g, 9.16 mmol), intermediate 50.1 (1.36 g, 9.16 mmol), and K₂CO₃ (1.52 g, 10.99 mmol) in toluene (7 mL) and N-methyl-2-pyrrolidinone (14 mL) under N₂ atmosphere, was reacted at 160° C. under azeotropic conditions for 3 h. Once cooled to r.t., the reaction was poured into EtOAc (40 mL), washed with H₂O (2×30 mL), brine (30 mL), dried over Na₂SO₄, and concentrated under reduced pressure. After chromatographic purification (PET/EtOAc from 95:5 to 70:30, v/v), the title intermediate 50.2 was obtained in 58% yield as pale yellow solid.

MS-ESI(+) m/z: 211.3 (M+H).

### Example 51: 3-(3-Methylphenoxy)aniline (51.4)

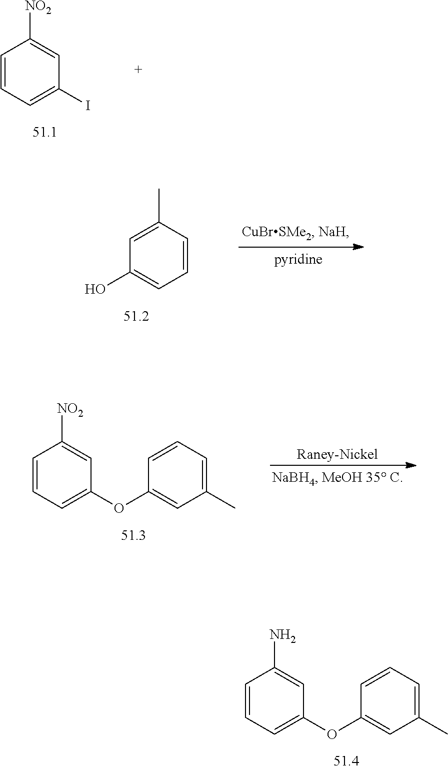

### Step 1: I-Methyl-3-(3-nitrophenoxy)benzene (51.3)

A mixture of intermediate 51.1 (1.00 g, 3.40 mmol), intermediate 51.2 (0.37 g, 3.40 mmol), NaH 60% in mineral oil (0.27 g, 6.80 mmol), and copper(I)bromide dimethyl sulfide complex (0.91 g, 4.42 mmol) in pyridine (7 mL), was stirred at 115° C. for 24 h. Once cooled to r.t., the reaction was poured into EtOAc (25 mL) and 3.0 M HCl (40 mL). The biphasic mixture was filtered through a celite pad under vacuum, washing the residual solid with EtOAc (2×10 mL). The two phases were separated and the aqueous phase was extracted with EtOAc (3×15 mL). The combined organic layers were washed with brine (50 mL), dried over Na₂SO₄, and concentrated under reduced pressure. After chromatographic purification (PET/EtOAc from 95:5 to 80:20, v/v), 0.40 g of intermediate 51.3 were obtained.

Yield: 51%.

MS-ESI(−) m/z: 228.6 (M+H).

### Step 2: 3-(3-Methylphenoxy)aniline (51.4)

The intermediate 51.3 was synthesized according to the procedure reported in Step 2 of Example 41 from intermediate 94.2 (0.40 g, 21.74 mmol), aq. Raney-Nickel suspension (0.5 mL), and sodium borohydride (0.13 mg, 3.49 mmol). After work up and chromatographic purification (PET/EtOAc from 90:10 to 70:30, v/v), 0.27 g of the title intermediate 51.4 were obtained as a colorless oil.

Yield: 77%

MS-ESI(+) m/z: 200.3 (M+H).

### Example 52: 3-(Pyridin-4-yloxy)aniline (52.3)

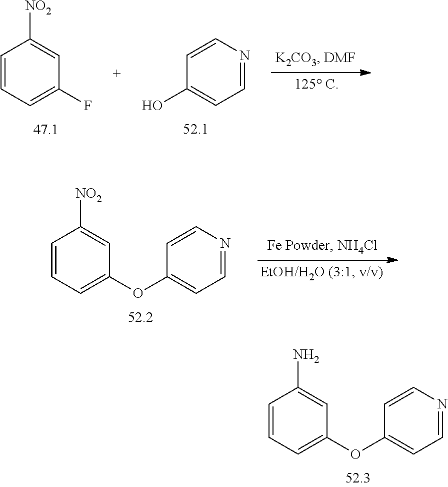 [SMILES: O=[N+]([O-])c1cccc(F)c1 ; Oc1ccncc1 ; O=[N+]([O-])c1cccc(Oc2ccncc2)c1 ; Nc1cccc(Oc2ccncc2)c1]

### Step 1: 4-(3-Nitrophenoxy)pyridine (52.2)

A mixture of intermediate 47.1 (1.00 g, 7.09 mmol), intermediate 52.1 (0.67 g, 7.09 mmol), and K₂CO₃ (1.96 g, 14.17 mmol) in DMF (7 mL) was stirred at 125° C. for 18 h. Once cooled to r.t., the reaction was poured into EtOAc (40 mL), washed with H₂O (2×30 mL), brine (30 mL), dried over Na₂SO₄, and concentrated under reduced pressure. After chromatographic purification (PET/EtOAc from 90:10 to 50:50, v/v), intermediate 52.2 was obtained in 22% yield as yellow solid.

MS-ESI(+) m/z: 217.3 (M+H).

### Step 2: 3-(Pyridin-4-yloxy)aniline (52.3)

The intermediate 52.3 was synthesized according to the procedure reported in Step 2 of Example 42 from intermediate 52.2 (1.10 g, 5.09 mmol), powdered iron (1.53 g, 27.48 mmol), and NH₄Cl (163 mg, 3.05 mmol) in EtOH/H₂O (3:1 v/v, 25 mL). After work up and chromatographic purification (CH₂Cl₂/MeOH from 98:2 to 90:10 v/v), 635 mg (3.41 mmol) of the title intermediate 52.3 as a yellow solid were obtained. Yield: 67%

MS-ESI(+) m/z: 187.3 (M+H)

### Example 53: 3-(Pyridin-2-yloxy)aniline (53.3)

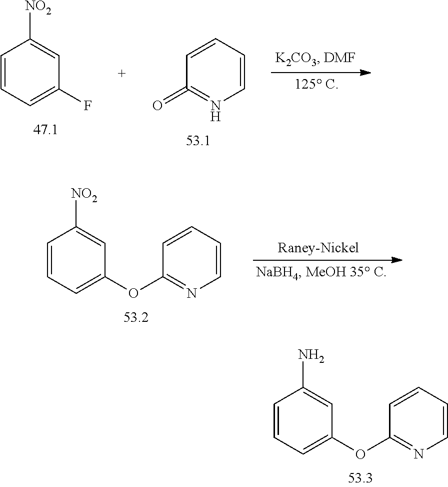

### Step 1: 2-(3-Nitrophenoxy)pyridine (53.2)

A mixture of intermediate 47.1 (1.00 g, 7.09 mmol), intermediate 53.1 (0.81 g, 8.50 mmol), and K₂C03 (1.96 g, 14.17 mmol) in DMF (10 mL) was stirred at 125° C. for 24 h. Once cooled to r.t. the reaction was poured into EtOAc (40 mL), washed with H₂O (40 mL), and brine (40 mL), dried over Na₂SO₄, and concentrated under reduced pressure. After chromatographic purification (CH₂Cl₂/MeOH from 99:1 to 96:4, v/v), 1.35 g of intermediate 53.2 were obtained.

Yield: 87%.

MS-ESI(+) m/z: 217.3 (M+H).

### Step 2: 3-(Pyridin-2-yloxy)aniline (53.3)

The intermediate 53.3 was synthesized according to the procedure reported in Step 2 of Example 41 from intermediate 53.2 (1.35 g, 6.24 mmol), powdered iron (1.89 g, 33.84 mmol), and NH₄Cl (203 mg, 3.80 mmol) in EtOH/H₂O (3:1 v/v, 40 mL). After work up and chromatographic purification (CH₂Cl₂/MeOH from 98:2 to 90:10 v/v), 628 mg (3.37 mmol) of the title intermediate 53.3 as a yellow solid were obtained. Yield: 54%

MS-ESI(+) m/z: 187.3 (M+H)

### Example 54: N-(Pyridin-3-yl)benzene-1,3-diamine (54.3)

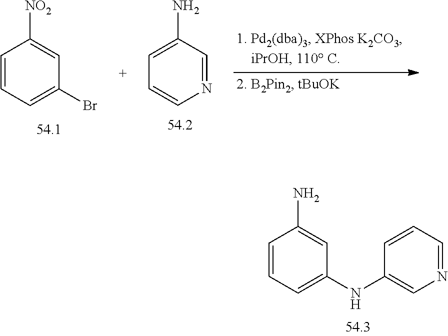 [SMILES: O=[N+]([O-])c1cccc(Br)c1 ; Nc1cccnc1 ; Nc1cccc(Nc2cccnc2)c1]

In a flame dried closed tube placed under N₂ atmosphere, intermediate 54.1 (1.05 g, 5.19 mmol), Pd₂(dba)₃ (43 mg, 0.05 mmol), Xantphos (136 mg, 0.23 mmol), and K₂CO₃ (1.30 g, 9.43 mmol) were added. The flask was evacuated under vacuum and refilled with N₂ 3 times. A solution of intermediate 54.2 (0.44 g, 4.18 mmol) in ⁱPrOH (6 mL) was then added, the tube was closed, and the mixture was stirred vigorously at 110° C. for 18 h. Once cooled to r.t., bis(pinacolato)diboron (3.59 g, 14.5 mmol) and ^{t}BuOK (0.85 g, 7.55 mmol) were cautiously added, the tube was closed and the mixture was stirred at 110° C. for 2 h. Once cooled to r.t., the reaction was poured into EtOAc/H₂O (50 mL, 2:1 v/v) and the biphasic mixture was filtered through a celite pad under vacuum, washing the residual solid with EtOAc (2×15 mL). Collecting the liquor, the two phases were separated and the aqueous phase was extracted with EtOAc (3×25 mL). The combined organic layers were washed with brine (50 mL), dried over Na₂SO₄ and concentrated under reduced pressure. After chromatographic purification (CH₂Cl₂/MeOH from 99:1 to 93:7, v/v), 0.34 g of the title intermediate 54.3 were obtained.

Yield: 38%.

MS-ESI(−) m/z: 186.1 (M+H).

### Example 55: N-(Pyridin-3-yl)benzene-1,4-diamine (55.2)

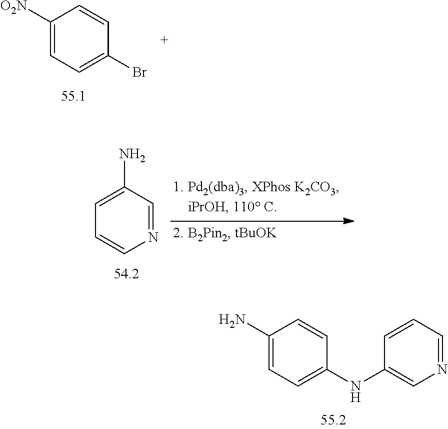 [SMILES: O=[N+]([O-])c1ccc(Br)cc1 ; Nc1cccnc1 ; Nc1ccc(Nc2cccnc2)cc1]

The intermediate 55.2 was synthesized according to the procedure reported in Example 54 from intermediate 55.1 (224 mg, 1.11 mmol), intermediate 54.2 (95 mg, 1.01 mmol), Pd₂(dba)₃ (9 mg, 0.01 mmol), Xantphos (29 mg, 0.0.5 mmol), and K₂CO₃ (279 mg, 2.02 mmol). In a second step, bis(pinacolato)diboron (1.024 g, 4.04 mmol) and tBuOK (226 mg, 2.02 mmol) were added. After work-up and chromatographic purification (CH₂Cl₂/MeOH from 99:1 to 93:7, v/v), 145 mg of the title intermediate 55.2 were obtained.

Yield: 77%.

MS-ESI(−) m/z: 186.1 (M+H).

### Example 56: 1-(Isoquinolin-5-yl)methanamine (56.4)

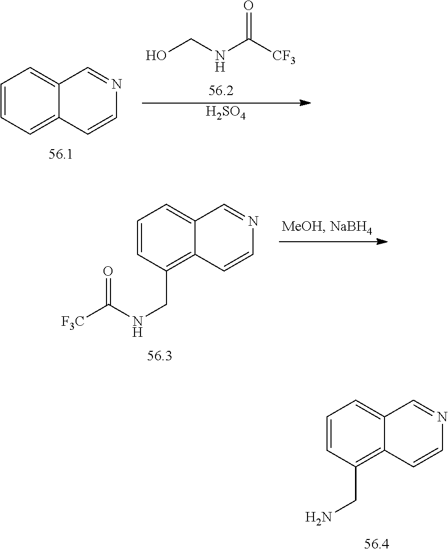

### Step 1: 2,2,2-Trifluoro-N-(isoquinolin-5-ylmethyl)acetamide (56.3)

Intermediate 56.2 (1.43 g, 10.00 mmol) was added portion wise to a stirred solution of intermediate 56.1 (1.29 g, 10.00 mmol) in cone. H₂SO₄ (50 mL) cooled at 0-5° C. Stirring was continued at 0-5° C. for 15 min, then the reaction was allowed to warm to r.t. and stirred for further 16 h. The mixture was cautiously poured into stirred ice (200 g), ammonia 28% (130 mL) was then added dropwise until a basic pH was reached. The aqueous mixture was extracted with CH₂Cl₂ (3×20 mL), the collected organic layers were washed with brine (40 mL), dried over Na₂SO₄, and concentrated under reduced pressure. After chromatographic purification (PET/EtOAc from 90:10 to 40:60), 0.68 g of intermediate 56.3 were obtained. Yield: 27%

MS-ESI(+) m/z: 255.3 (M+H).

### Step 2: 1-(Isoquinolin-5-yl)methanamine (56.4)

Sodium borohydride (0.32 g, 8.40 mmol) was added portion wise to a stirred solution of intermediate 56.3 (0.65 g, 2.50 mmol) in MeOH (25 mL). After 1 h, the volatiles were removed under reduced pressure, the crude was dissolved in CH₂Cl₂ (20 mL). The resulting solution was washed with brine (20 mL), dried over Na₂SO₄, and concentrated under reduced pressure, to afford the title intermediate 56.4 as colorless oil in nearly quantitative yield.

MS-ESI(+) m/z: 159.3 (M+H).

### Example 57: 3-[(6-Methylpyridin-3-yl)oxy]aniline (57.2)

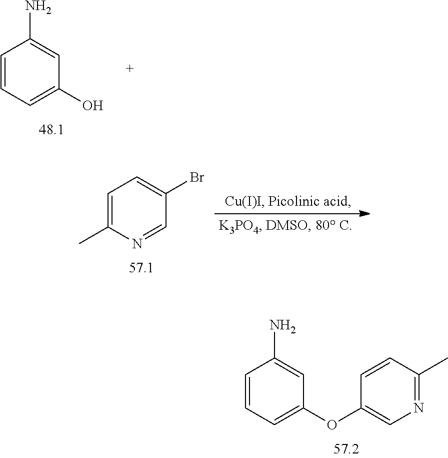

Intermediate 57.2 was synthesized according to the procedure reported in Example 49 starting from intermediate 48.1 (200 mg, 1.16 mmol), intermediate 57.1 (152 mg, 1.40 mmol), Cu(I)I (11 mg, 0.06 mmol), K₃PO₄ (494 mg, 2.33 mmol), and picolinic acid (14 mg, 0.12 mmol) in DMSO (3 mL) After workup and chromatographic purification (PET/EtOAc from 95:5 to 70:30, v/v), the title intermediate 57.2 (115 mg, 0.57 mmol) was obtained in 49% yield.

MS-ESI(+) m/z: 201.2 (M+H).

### Example 58: 3-{[6-(Trifluoromethyl)pyridin-3-yl]oxy}aniline (58.2)

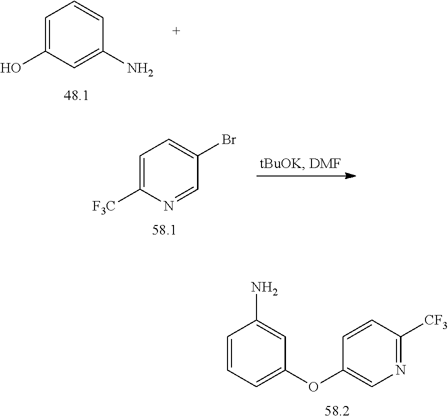 [SMILES: Nc1cccc(O)c1 ; FC(F)(F)c1ccc(Br)cn1 ; Nc1cccc(Oc2ccc(C(F)(F)F)nc2)c1]

A solution of intermediate 48.1 (300 mg, 2.75 mmol) in dry DMF (5 mL) was degassed by evacuation of the head space and backfilling with N₂ (3 times), tBuOK (370 mg, 2.30 mmol) was then added and the resulting suspension was stirred at r.t. for 30 minutes. Intermediate 58.1 (621 mg, 2.75 mmol) was then added and the mixture was reacted under magnetic stirring at 105° C. for 18 h. The mixture was poured into H₂O (50 mL) and extracted with CH₂Cl₂ (3×10 mL). The combined organic layers were washed with H₂O (50 mL) and brine (50 mL), dried over anhydrous Na₂SO₄, and evaporated to dryness. After purification by flash chromatography (CH₂Cl₂/MeOH, from 100% CH₂Cl₂ to 9:1 v/v CH₂Cl₂/MeOH) the title intermediate 58.2 (320 mg, 1.26 mmol) was obtained as a colorless oil. Yield: 46%. MS-ESI(+) m/z: 255.5 (M+H); MS-ESI(−) m/z: 253.3 (M−H).

### Example 59: 4-(6-Fluoropyridin-3-yl)aniline (59.3)

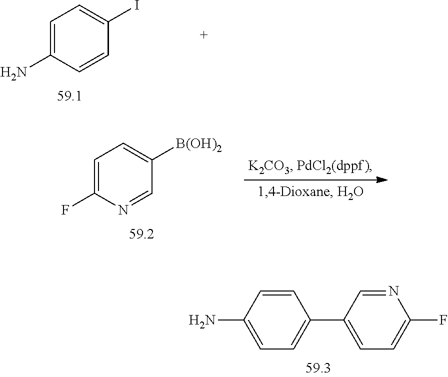 [SMILES: Nc1ccc(I)cc1 ; OB(O)c1ccc(F)nc1 ; Nc1ccc(-c2ccc(F)nc2)cc1]

The intermediate 59.3 was synthesized according to the procedure reported in Example 40 from intermediate 59.1 (500 mg, 2.28 mmol), intermediate 59.2 (386 mg, 2.73 mmol), K₂CO₃ (1.55 g, 11.28 mmol), and [1,1′-bis(diphenylphosphino)ferrocene]palladium(II) dichloride (103 mg, 0.14 mmol) in 1,4-dioxane (14 mL) and H₂O (5 mL). After work up and chromatographic purification, 200 mg of title intermediate 59.3 were obtained.

Yield: 38%.

MS-ESI(+) m/z: 189.6 (M+H).

### Example 60: trans-3-(4-Fluorophenoxy)cyclobutanamine hydrochloride (60.4)

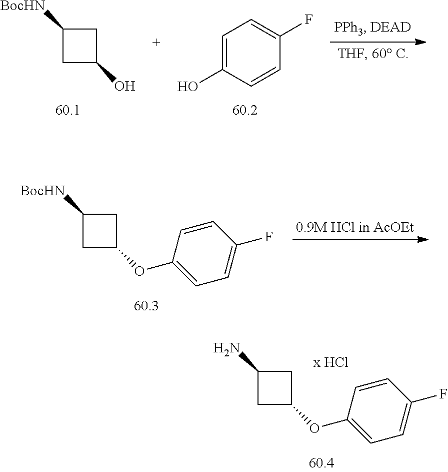 [SMILES: CC(C)(C)OC(=O)N[C@H]1C[C@@H](O)C1 ; Oc1ccc(F)cc1 ; CC(C)(C)OC(=O)N[C@H]1C[C@H](Oc2ccc(F)cc2)C1 ; N[C@H]1C[C@H](Oc2ccc(F)cc2)C1]

### Step 1: tert-Butyl [trans-3-(4-fluorophenoxy)cyclobutyl]carbamate (60.3)

Diethyl azodicarboxylate (0.45 mL, 2.89 mmol) and intermediate 60.2 (325 mg, 2.89 mmol) were added to a stirred solution of intermediate 60.1 (500 mg, 2.67 mmol) and triphenylphospine (760 mg, 2.89 mmol) in THF (20 mL) held under N₂ atmosphere and cooled at 0-5° C. The mixture was slowly heated up to 60° C. and reacted for 16 h. Volatiles were removed under reduced pressure, and the crude was dissolved in CH₂Cl₂ (50 mL), washed with 2.0 M aq. NaOH (2×10 mL), brine (20 mL), dried over Na₂SO₄, and concentrated under reduced pressure. After chromatographic purification (PET/EtOAc from 95:5 to 50:50, v/v), 220 mg of the intermediate 60.3 were obtained as a white solid.

Yield: 29%.

MS-ESI(−) m/z: 280.4 (M−H).

### Step 2: trans-3-(4-Fluorophenoxy)cyclobutanamine hydrochloride (60.4)

A solution of intermediate 60.3 (200 mg, 0.71 mmol) in 0.9 M HCl in EtOAc (3.15 mL, 2.84 mmol) was stirred at r.t. for 16 h. Volatiles were removed under reduced pressure, to give 153 mg of the title intermediate 60.4.

Yield: 99%.

MS-ESI(+) m/z: 182.3 (M+H).

### Example 61: trans-3-(4-Methylphenoxy)cyclobutanamine hydrochloride (61.3)

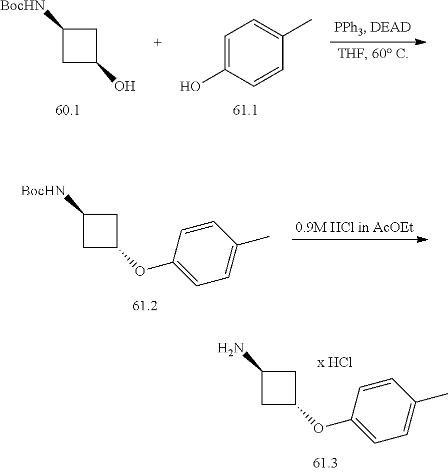

### Step 1: tert-Butyl [trans-3-(4-methylphenoxy)cyclobutyl]carbamate 057.2)

The intermediate 61.2 was synthesized according to the procedure reported in Step 1 of Example 60 from intermediate 60.1 (400 mg, 2.13 mmol), triphenylphospine (616 mg, 2.34 mmol), DIAD (0.46 mL, 2.34 mmol), and intermediate 61.1 (253 mg, 2.34 mmol) in THF (15 mL). After work-up and chromatographic purification (/EtOAc from 95:5 to 70:00, v/v), 250 mg of the intermediate 61.2 were obtained.

Yield: 42%.

MS-ESI(−) m/z: 276.3 (M+H).

### Step 2: trans-3-(4-Methylphenoxy)cyclobutanamine hydrochloride (61.3)

The intermediate 61.3 was synthesized according to the procedure reported in Step 2 of Example 60 from intermediate 61.2 (250 mg, 0.90 mmol) and 0.9 M HCl in EtOAc (4.00 mL, 6.80 mmol). 184 mg of the title intermediate 61.3 were obtained.

Yield: 96%.

MS-ESI(−) m/z: 178.3 (M+H).

### Example 62: trans-3-(Pyridin-3-yloxy)cyclobutanamine dihydrochloride (62.3)

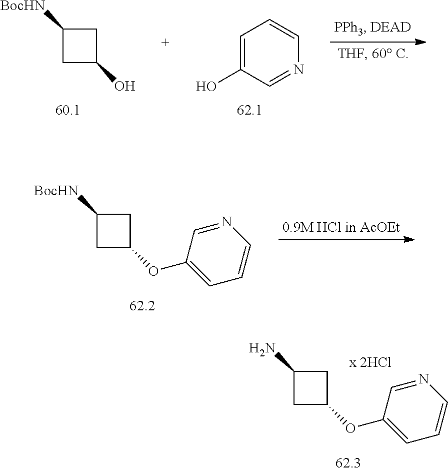 [SMILES: CC(C)(C)OC(=O)N[C@H]1C[C@@H](O)C1 ; Oc1cccnc1 ; CC(C)(C)OC(=O)N[C@H]1C[C@H](Oc2cccnc2)C1 ; N[C@H]1C[C@H](Oc2cccnc2)C1]

### Step 1: tert-Butyl [trans-3-(pyridin-3-yloxy)cyclobutyl]carbamate (62.2)

The intermediate 62.2 was synthesized according to the procedure reported in Step 1 of Example 60 from intermediate 60.1 (350 mg, 1.87 mmol), triphenylphospine (539 mg, 2.05 mmol), DIAD (0.40 mL, 2.05 mmol) and intermediate 62.1 (195 mg, 2.05 mmol) in THF (14 mL). After work-up and chromatographic purification (CH₂Cl₂/MeOH from 99:1 to 93:7, v/v), 260 mg of the intermediate 62.2 were obtained.

Yield: 52%.

MS-ESI(+) m/z: 265.3 (M+H).

### Step 2: trans-3-(Pyridin-3-yloxy)cyclobutanamine dihydrochloride (62.3)

The intermediate 62.3 was synthesized according to the procedure reported in Step 2 of Example 60 from intermediate 62.2 (250 mg, 0.95 mmol) and 0.9 M HCl in EtOAc (4.22 mL, 3.80 mmol). 204 mg of title intermediate 62.3 were obtained.

Yield: 81%.

MS-ESI(+) m/z: 165.3 (M+H).

### Example 63: trans-3-[(6-Methylpyridin-3-yl)oxy]cyclobutanamine dihydrochloride (63.3)

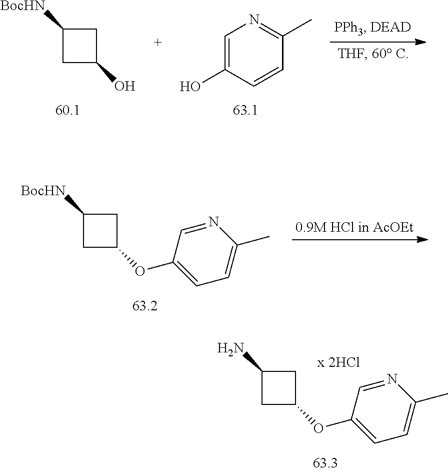

### Step 1: tert-Butyl [trans-3-[(6-methylpyridin-3-yl)oxy]cyclobutyl]carbamate (63.2)

The intermediate 63.2 was synthesized according to the procedure reported in Step 1 of Example 60 from intermediate 60.1 (350 mg, 1.87 mmol), triphenylphospine (580 mg, 2.24 mmol), DIAD (0.44 mL, 2.24 mmol), and 63.1 (244 mg, 2.24 mmol) in THF (14 mL). After work-up and chromatographic purification (CH₂Cl₂/MeOH from 99:1 to 93:7, v/v), 362 mg of the intermediate 63.2 were obtained.

Yield: 69%.

MS-ESI(+) m/z: 279.4 (M+H).

### Step 2: trans-3-[(6-Methylpyridin-3-yl)oxy]cyclobutanamine dihydrochloride (63.3)

Intermediate 63.2 (350 mg, 1.26 mmol) was treated with 0.9 M HCl in EtOAc (45.6 mL, 5.04 mmol) at r.t. for 16 h and then at 50° C. for 2 h. The obtained suspension was centrifugated and the supernatant was removed. The collected white powder was washed with Et₂O (2×5 mL) and dried under vacuum, to give the title intermediate 63.3 in nearly quantitative yield.

MS-ESI(+) m/z: 179.4 (M+H).

### Example 64: trans-3-[(6-Fluoropyridin-3-yl)oxy]cyclobutanamine dihydrochloride (64.3)

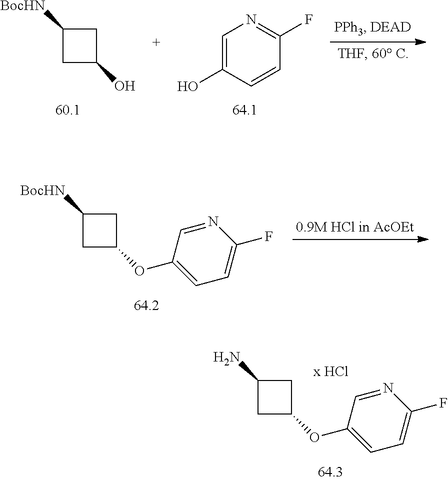 [SMILES: CC(C)(C)OC(=O)N[C@H]1C[C@@H](O)C1 ; Oc1ccc(F)nc1 ; CC(C)(C)OC(=O)N[C@H]1C[C@H](Oc2ccc(F)nc2)C1 ; N[C@H]1C[C@H](Oc2ccc(F)nc2)C1]

### Step 1: tert-Butyl [trans-3-[(6-fluoropyridin-3-yl)oxy]cyclobutyl]carbamate (64.2)

The intermediate 64.2 was synthesized according to the procedure reported in Step 1 of Example 60 from intermediate 60.1 (350 mg, 1.87 mmol), triphenylphospine (539 mg, 2.05 mmol), DIAD (0.40 mL, 2.05 mmol), and intermediate 64.1 (232 mg, 2.05 mmol) in THF (15 mL). After work-up and chromatographic purification (CH₂Cl₂/MeOH from 99:1 to 95:5, v/v), 600 mg of crude intermediate 64.2 were obtained, which was used such as for the next step.

### Step 2: trans-3-[(6-Fluoropyridin-3-yl)oxy]cyclobutanamine dihydrochloride (64.3)

The intermediate 64.3 was synthesized according to the procedure reported in Step 2 of Example 60. After washing with Et₂O and removal of residual volatiles under vacuum, 210 mg of title intermediate 64.3 were obtained.

Yield: 23% from intermediate 10.1.

MS-ESI(+) m/z: 182.3 (M+H).

### EXEMPLIFICATION OF TITLE COMPOUNDS

### Example 64: N-(Isoquinolin-5-yl)-4-phenyl-2,5-dihydro-1H-pyrrole-3-carboxamide (Compound I-1)

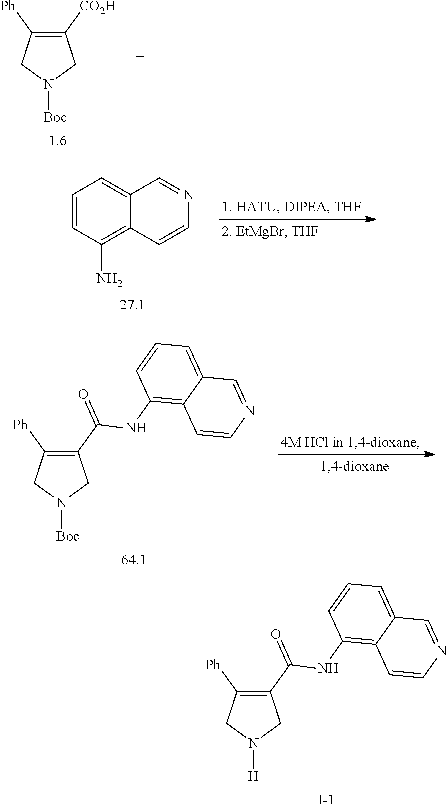 [SMILES: CC(C)(C)OC(=O)N1CC(C(=O)O)=C(c2ccccc2)C1 ; Nc1cccc2cnccc12 ; CC(C)(C)OC(=O)N1CC(C(=O)Nc2cccc3cnccc23)=C(c2ccccc2)C1 ; O=C(Nc1cccc2cnccc12)C1=C(c2ccccc2)CNC1 ; [I-]]

### Step 1: tert-Butyl 3-(isoquinolin-5-ylcarbamoyl)-4-phenyl-2,5-dihydro-1H-pyrrole-1-carboxylate (64.1)

DIPEA (270 μL, 1.55 mmol) and HATU (236 mg, 0.62 mmol) were added to a stirred solution of intermediate 1.6 (150 mg, 0.52 mmol) in THF (3 mL) under a N₂ atmosphere, and the resulting fine suspension was stirred at r.t. for 45 min. In a second flask, intermediate 27.1 (112 mg, 0.78 mmol) was dissolved in THF (3 mL) under a N₂ atmosphere, 3.0 M EtMgBr in Et₂O (0.52 mL, 1.55 mmol) was then quickly added dropwise. The resulting yellow orange suspension was stirred for 15 min and then to this solution the mixture coming from the first flask was added dropwise. The resulting mixture was vigorously stirred at r.t. for 4 h. The crude was poured into EtOAc (10 mL), washed with H₂O (10 mL), 0.5 M aqueous citric acid (10 mL), brine (10 mL), dried over Na₂SO₄, and concentrated under reduced pressure. The crude was purified by flash chromatography (DCM/MeOH from 99:1 to 95:5 v/v). Intermediate 64.1 (79 mg, 0.19 mmol) was obtained as a colorless oil. Yield: 24%. MS-ESI(+) m/z: 416.6 (M+H); MS-ESI(−) m/z: 414.6 (M−H).

### Step 2: N-(Isoquinolin-5-yl)-4-phenyl-2,5-dihydro-1H-pyrrole-3-carboxamide (Compound I-1)

The intermediate 64.1 (79 mg, 0.19 mmol) was dissolved in 1,4-dioxane (2.5 mL) and treated with 4.0 M HCl in 1,4-dioxane (0.47 mL, 1.90 mmol) for 24 h. The volatiles were removed under reduced pressure, the crude was then dissolved in H₂O (5 mL) and washed with Et₂O (2×5 mL). The aqueous phase was basified by adding aq. NaHCO₃ ss and extracted with DCM/MeOH (9:1 v/v, 3×10 mL). The collected organic layers were washed with brine (15 mL), dried over Na₂SO₄, and concentrated under reduced pressure. The title compound I-1 (36 mg, 0.11 mmol) was obtained as a pale yellow powder. Yield: 58%. ¹H-NMR (400 MHz, DMSO-d₆) δ 3.20-3.55 (m, 4H), 7.30-7.50 (m, 6H), 7.69 (t, J=9.2 Hz, 1H), 7.91-7.97 (m, 2H), 8.38 (d, J=5.9 Hz, 1H), 9.28 (s, 1H), 10.12 (s. 1H). UHPLC purity: ≥95%. MS-ESI(+) m/z: 316.5 (M+H); MS-ESI(−) m/z: 314.5 (M−H).

### Example 65: (±)-trans-O-(4-Phenylpyrrolidin-3-yl) isoquinolin-5-ylcarbamothioate (Compound I-2)

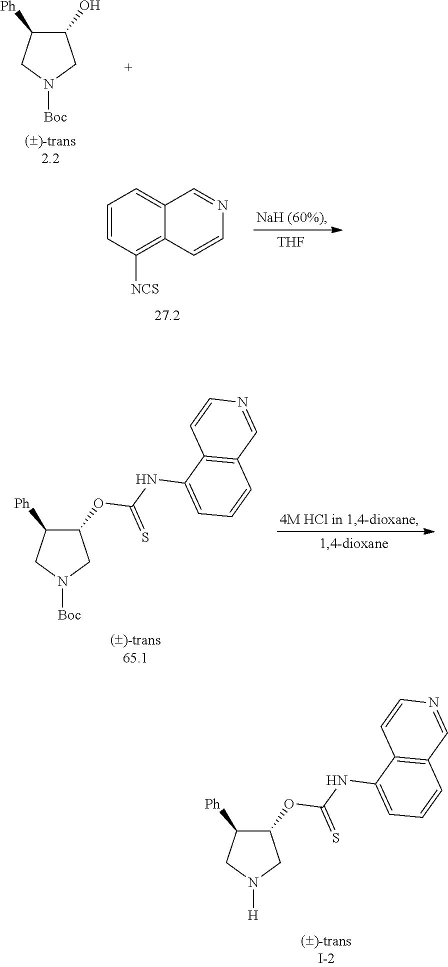

### Step 1: tert-Butyl (±)-trans-4-phenyl-3-[(isoquinolin-5-ylcarbamothioyl)oxy]pyrrolidine-1-carboxylate (65.1)

NaH (60% in mineral oil) (22 mg, 0.54 mmol) in THF (4 mL) was added to a stirred solution of intermediate 2.2 (141 mg, 0.54 mmol) in dry THF (2 mL) under a N₂ atmosphere, and the resulting mixture was reacted at r. t. for 30 min. A solution of intermediate 27.2 (100 mg, 0.54 mmol) in dry THF (2 mL) was then added and the mixture was stirred at r.t. for 16 h. The mixture was then poured into H₂O (20 mL) and extracted with EtOAc (3×10 mL). The combined organic layers were washed with H₂O (20 mL) and brine (20 mL), dried over anhydrous Na₂SO₄, and evaporated to dryness. The crude intermediate 65.1 was used such as for the next step. MS-ESI(+) m/z: 450.2 (M+H).

### Step 2: (±)-trans-O-(4-Phenylpyrrolidin-3-yl) isoquinolin-5-ylcarbamothioate (Compound I-2)

The intermediate 65.1 (crude from previous step, 0.54 mmol) was dissolved in THF (10 mL) and MeOH (1 mL). The resulting solution was treated with 37% aq. HCl (233 μL, 2.685 mmol) at 55° C. for 6 h. Once cooled to r.t., the opalescent solution, so obtained, was poured into H₂O (20 mL) and washed with Et₂O (2×15 mL). The aqueous layer was basified by adding aq. NaHCO₃ ss and extracted with DCM/MeOH (9:1 v/v, 3×15 mL). The collected organic layers were dried over Na₂SO₄ and concentrated under reduced pressure. The crude was purified by reverse phase flash chromatography (H₂O/MeCN from 70:30 to 0:100 v/v). The title compound I-2 (71 mg, 0.20 mmol) was obtained as a yellow powder. Yield: 38%. ¹H-NMR (400 MHz, CD₃OD) δ 2.84-3.63 (m, 5H, two rotamers), 5.81-5.85 (m, 1H, two rotamers), 7.01-7.35 (m, 6H), 7.65-8.08 (m, 5H), 8.42-8.50 (m, 1H, two rotamers), 9.23+9.28 (s+s, 1H, two rotamers). UHPLC purity: ≥90%. MS-ESI(+) m/z: 349.9 (M+H); MS-ESI(−) m/z: 347.8 (M−H).

### Example 66: (±)-cis-O-(4-Phenylpyrrolidin-3-yl) isoquinolin-5-ylcarbamothioate (Compound I-3)

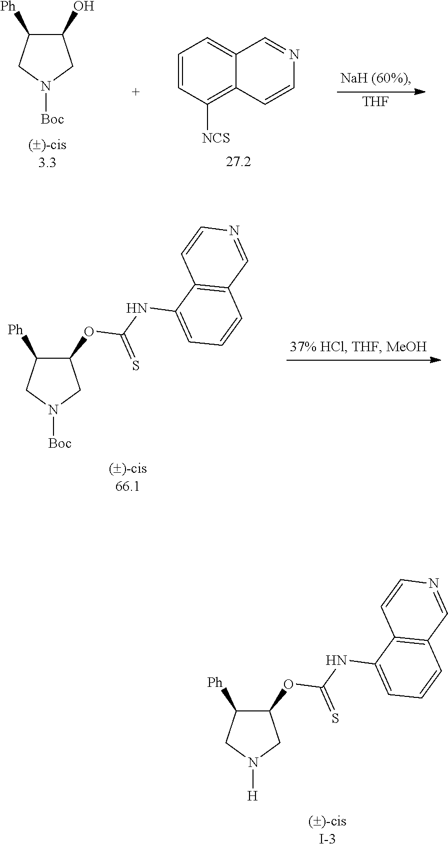

### Step 1: tert-Butyl (±)-cis-4-phenyl-3-[(isoquinolin-5-ylcarbamothioyl)oxy]pyrrolidine-1-carboxylate (66.1)

NaH (60% in mineral oil) (22 mg, 0.54 mmol) was added to a stirred solution of intermediate 3.3 (141 mg, 0.54 mmol) in dry THF (2 mL) under a N₂ atmosphere, and the resulting mixture was reacted at r.t. for 30 min. A solution of intermediate 27.2 (100 mg, 0.54 mmol) in dry THF (2 mL) was then added and the mixture stirred at r.t. for 16 h. The mixture was then poured into H₂O (20 mL) and extracted with EtOAc (3×10 mL). The combined organic layers were washed with H₂O (20 mL) and brine (20 mL), dried over anhydrous Na₂SO₄, and evaporated to dryness. The crude intermediate 66.1 was used such as for the next step. MS-ESI(+) m/z: 450.2 (M+H).

### Step 2: (±)-cis-O-(4-Phenylpyrrolidin-3-yl) isoquinolin-5-ylcarbamothioate (Compound I-3)

Compound I-3 was synthesized from intermediate 66.1 (crude of previous step, 0.54 mmol) which was reacted with 37% aq. HCl (233 μL, 2.685 mmol) in THF (10 mL) and MeOH (1 mL). The title compound I-3 (17 mg, 0.05 mmol) was obtained as a pale orange powder after reverse phase chromatographic purification. Yield: 9%. ¹H-NMR (400 MHz, CD₃OD) δ 3.01-3.77 (m, 5H, two rotamers), 5.95-5.99+6.28-6.32 (m+m, 1H, two rotamers), 6.68-7.63 (m, 10H, two rotamers), 8.03-8.07 (m, 1H, two rotamers), 8.33-8.40 (m, 1H, two rotamers), 9.23-9.28 (m, 1H, two rotamers). UHPLC purity: ≥90%. MS-ESI(+) m/z: 349.9 (M+H); MS-ESI(−) m/z: 347.7 (M−H).

### Example 67: (±)-trans-4-Phenylpyrrolidin-3-yl isoquinolin-5-ylcarbamate (Compound I-4)

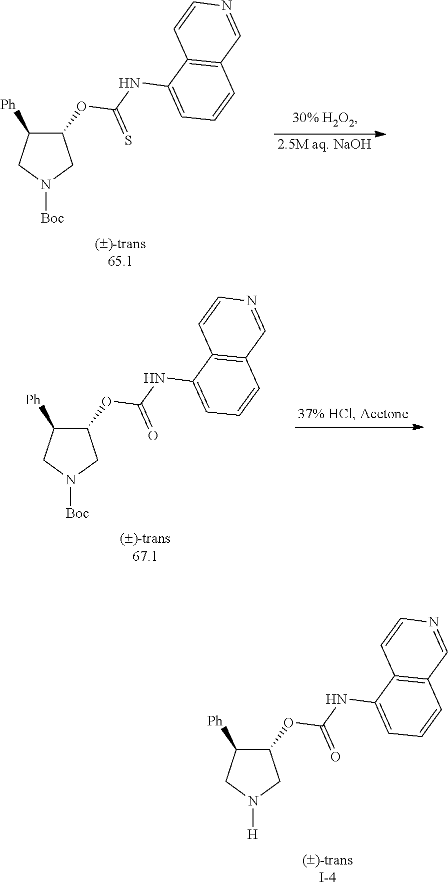 [SMILES: CC(C)(C)OC(=O)N1C[C@@H](OC(=S)Nc2cccc3cnccc23)[C@H](c2ccccc2)C1 ; "2.5M aq. NaOH" ; CC(C)(C)OC(=O)N1C[C@@H](OC(=O)Nc2cccc3cnccc23)[C@H](c2ccccc2)C1 ; O=C(Nc1cccc2cnccc12)O[C@@H]1CNC[C@H]1c1ccccc1]

### Step 1: tert-Butyl (±)-trans-4-phenyl-3-[(isoquinolin-5-ylcarbamoyl)oxy]pyrrolidine-1-carboxylate (67.1)

H₂O₂ (30% w/w in H₂O) (995 μL, 9.74 mmol) was added dropwise to a stirred suspension of 65.1 (100 mg, 0.22 mmol) in 10% aq. NaOH (3 mL) cooled to 0-5° C., and the resulting suspension was allowed to gradually warm to r.t. then reacted at r.t. for 16 h. The mixture was diluted with H₂O (10 mL) and extracted with EtOAc (3×10 mL). The combined organic layers were washed with H₂O (20 mL) and brine (20 mL), dried over anhydrous Na₂SO₄, and evaporated to dryness. The residue was purified by flash chromatography (DCM/MeOH, from 100% DCM, to 95:5 v/v DCM/MeOH). Intermediate 67.1 (90 mg, 0.21 mmol) was obtained as a white solid. Yield: 94%. MS-ESI(+) m/z: 433.7 (M+H); MS-ESI(−) m/z: 431.7 (M−H).

### Step 2: (±)-trans-4-Phenylpyrrolidin-3-yl isoquinolin-5-ylcarbamate (Compound I-4)

Compound I-4 was synthesized starting from intermediate 67.1 (90 mg, 0.21 mmol) by reaction with 37% aq. HCl (173 μL, 2.08 mmol) in acetone (4 mL). The title compound I-4 (47 mg, 0.14 mmol) was obtained as a white solid after flash chromatography purification of the crude (DCM/MeOH, from 100% DCM, to 85:15 v/v DCM/MeOH). Yield: 67%. ¹H-NMR (400 MHz, CD₃OD) δ 3.06-3.09 (m, 1H), 3.23-3.28 (m, 1H), 3.44-3.56 (m, 3H), 5.29-5.30 (m, 1H), 7.25-7.37 (m, 5H), 7.68 (t, J=8.0 Hz, 1H), 7.94 (t, J=8.0 Hz, 2H), 7.97-8.01 (m, 1H), 8.45 (d, J=6.0 Hz, 1H), 9.24 (s, 1H). UHPLC purity: ≥95%. MS-ESI(+) m/z: 334.3 (M+H).

### Example 68: (±)-trans-3-{2-[(4-Phenylpyrrolidin-3-yl)oxy]-1,3-thiazol-4-yl}pyridine (Compound I-5)

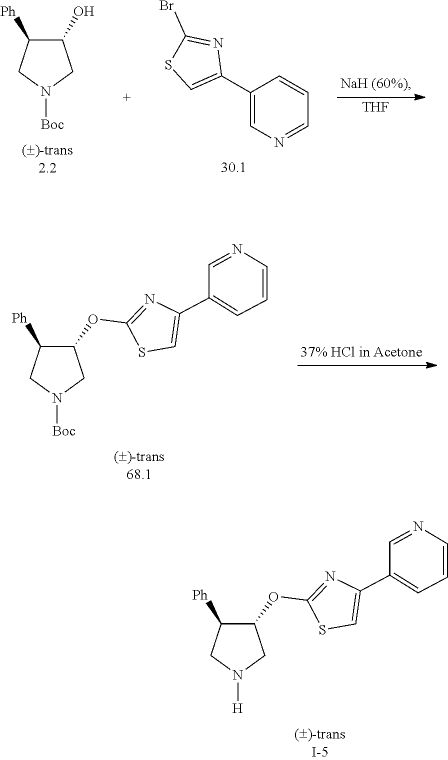 [SMILES: CC(C)(C)OC(=O)N1C[C@@H](O)[C@H](c2ccccc2)C1 ; Brc1nc(-c2cccnc2)cs1 ; CC(C)(C)OC(=O)N1C[C@@H](Oc2nc(-c3cccnc3)cs2)[C@H](c2ccccc2)C1 ; c1ccc([C@@H]2CNC[C@H]2Oc2nc(-c3cccnc3)cs2)cc1 ; "THF"]

### Step 1: tert-Butyl (±)-trans-3-phenyl-4-{[4-(pyridin-3-yl)-1,3-thiazol-2-yl]oxy}pyrrolidine-1-carboxylate (68.1)

Intermediate 2.2 (109 mg, 0.41 mmol) was added to a stirred suspension of NaH (60% in mineral oil) (36 mg, 0.91 mmol) in dry THF (5 mL) under a N₂ atmosphere and the resulting mixture was reacted at r.t. for 15 min. A solution of intermediate 30.1 (100 mg, 0.41 mmol) in dry THF (5 mL) was then added dropwise and the mixture was stirred at 60° C. for 18 h. The mixture was poured into H₂O (30 mL) and extracted with EtOAc (3×10 mL). The combined organic layers were washed with H₂O (20 mL) and brine (20 mL), dried over anhydrous Na₂SO₄, and evaporated to dryness. The residue was purified by flash chromatography (DCM/MeOH, from 100% DCM, to 95:5 v/v DCM/MeOH). Intermediate 68.1 (140 mg, 0.33 mmol) was obtained as a brownish powder. Yield: 80%. MS-ESI(+) m/z: 424.3 (M+H).

### Step 2: (±)-trans-3-{2-[(4-Phenylpyrrolidin-3-yl)oxy]-1,3-thiazol-4-yl}pyridine (Compound I-5)

Compound I-5 was synthesized according to the procedure described in Step 2 of Example 67 starting from intermediate 68.1 (140 mg, 0.33 mmol) which was reacted with 37% aq. HCl (354 μL, 4.25 mmol) in acetone (4 mL). The title compound I-5 (38 mg, 0.12 mmol) was obtained as a yellow oil after flash chromatography purification of the crude (DCM/MeOH, from 100% DCM, to 85:15 v/v DCM/MeOH). Yield: 36%. ¹H-NMR (400 MHz, CDCl₃) δ 3.05-3.10 (m, 1H), 3.46-3.48 (m, 2H), 3.55-3.59 (m, 1H), 3.67-3.72 (m, 1H), 5.50-5.51 (m, 1H), 6.97 (s, 1H), 7.25-7.39 (m, 6H), 7.91 (d, J=7.9 Hz, 1H), 8.51 (t, J=4.8 Hz, 1H), 8.94 (s, 1H). UHPLC purity: ≥95%. MS-ESI(+) m/z: 324.2 (M+H).

### Example 69: (±)-trans-1-Isoquinolin-5-yl-3-(1-benzyl-4-phenylpyrrolidin-3-yl)thiourea (Compound I-6)

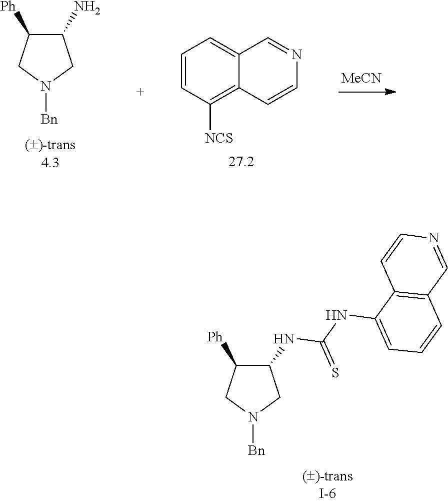

A mixture of intermediate 4.3 (280 mg, 1.11 mmol) and intermediate 27.2 (206 mg, 1.11 mmol) in MeCN (10 mL) was stirred at r.t. for 3 h. The obtained suspension was filtered under vacuum, washing the solid with cold MeCN. The title compound I-6 (375 mg, 0.86 mmol) was obtained as a white powder after desiccation under reduced pressure at 45° C. for 18 h. Yield: 77%. ¹H-NMR (400 MHz, DMSO-d₆) δ 2.47-2.52 (m, 2H), 2.90-2.94 (m, 1H), 2.96-3.00 (m, 1H), 3.20-3.29 (m, 1H), 3.49 (d, J=13.0 Hz, 1H), 3.58 (d, J=12.7 Hz, 1H), 4.72-4.78 (m, 1H), 7.16-7.30 (m, 10H), 7.54-7.59 (m, 2H), 7.67-7.70 (m, 1H), 7.90 (d, J=8.03 Hz, 1H), 8.11 (d, J=5.5 Hz, 1H), 8.37 (d, J=4.9 Hz, 1H), 9.23 (s, 1H), 9.55 (s, 1H). UHPLC purity: ≥95%. MS-ESI(+) m/z: 439.3 (M+H); MS-ESI(−) m/z: 437.3 (M−H).

### Example 70: (±)-trans-1-Isoquinolin-5-yl-3-(4-phenylpyrrolidin-3-yl)thiourea (Compound I-7)

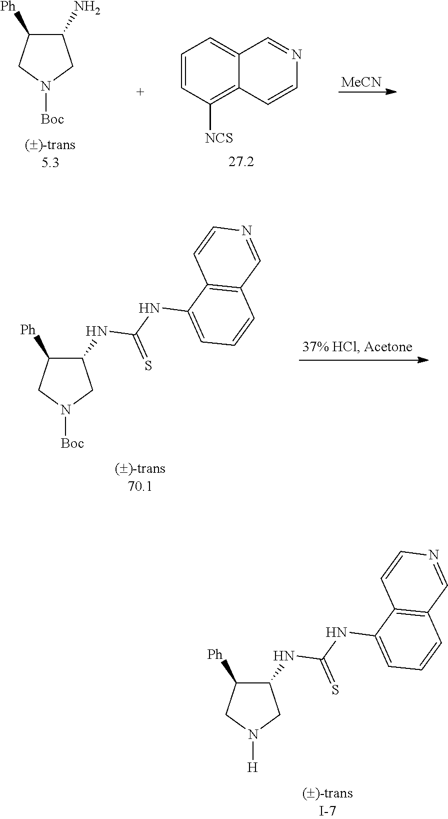

### Step 1: tert-Butyl (±)-trans-3-phenyl-4-[(isoquinolin-5-ylcarbamothioyl)amino]pyrrolidine-1-carboxylate (70.1)

Intermediate 70.1 was synthesized according to the procedure described in Example 69 starting from intermediate 5.3 (174 mg, 0.66 mmol) which was reacted with intermediate 27.2 (174 mg, 0.66 mmol) in MeCN (6 mL). Intermediate 70.1 (132 mg, 0.29 mmol) was obtained as a yellow oil after flash chromatography purification (DCM/MeOH, from 100% DCM, to 9:1 v/v DCM/MeOH). Yield: 44%. MS-ESI(+) m/z: 447.7 (M+H); MS-ESI(−) m/z: 449.6 (M−H).

### Step 2: (±)-trans-1-Isoquinolin-5-yl-3-(4-phenylpyrrolidin-3-yl)thiourea (Compound I-7)

Compound I-7 was synthesized starting from intermediate 70.1 (40 mg, 0.09 mmol) by reaction with 37% aq. HCl (74 μL, 0.89 mmol) in acetone/H₂O (3:1 v/v, 3 mL). The title compound I-7 (14 mg, 0.04 mmol) was obtained as a white powder after flash chromatography purification of the crude (DCM/MeOH, from 100% DCM, to 85:15 v/v DCM/MeOH). Yield: 44%. ¹H-NMR (400 MHz, DMSO-d₆) δ 3.28-3.3.30 (m, 1H), 3.52-3.59 (m, 1H), 3.64-3.88 (m, 3H), 4.82-4.85 (m, 1H), 7.23 (d, J=7.3 Hz, 1H), 7.32 (t, J=7.3 Hz, 2H), 7.37-7.48 (m, 2H), 7.54-7.65 (m, 2H), 7.77-7.97 (m, 3H), 8.35-8.44 (m, 1H), 9.25 (s, 1H), 10.62 (brs, 1H), 12.12 (brs, 1H). UHPLC purity: ≥90%. MS-ESI(−) m/z: 449.8 (M−H); MS-ESI(+) m/z: 347.6 (M+H).

### Example 71: (±)-trans-N-(4-Phenylpyrrolidin-3-yl)isoquinoline-5-sulfonamide (Compound I-8)

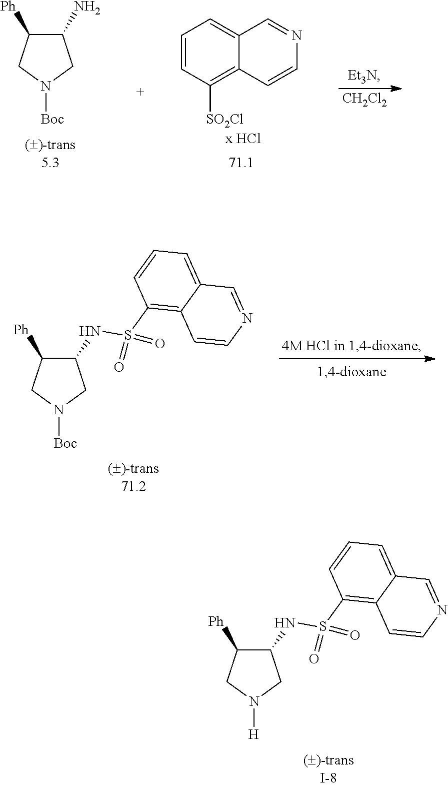

### Step 1: tert-Butyl (±)-trans-4-phenyl-3-[(isoquinolin-5-ylsulfonyl)amino]pyrrolidine-1-carboxylate (71.2)

Et₃N (161 μL, 1.14 mmol) and intermediate 71.1 (101 mg, 0.38 mmol) were added to a stirred solution of intermediate 5.3 (100 mg, 0.38 mmol) in DCM (5 mL), and the resulting mixture was stirred at r.t. for 24 h. The reaction was poured into H₂O (10 mL) and extracted with DCM (3×10 mL). The collected organic layers were dried over Na₂SO₄ and concentrated under reduced pressure. The crude was purified by flash chromatography (DCM/MeOH from 98:2 to 90:10 v/v), to give intermediate 71.2 (44 mg, 0.10 mmol) as a colorless oil. Yield: 26%. MS-ESI(+) m/z: 455.4 (M+H); MS-ESI(−) m/z: 452.4 (M−H).

### Step 2: (±)-trans-N-(4-Phenylpyrrolidin-3-yl)isoquinoline-5-sulfonamide (Compound I-8)

Intermediate 71.2 (44 mg, 0.09 mmol) was dissolved in 1,4-dioxane (3 mL) and H₂O (0.5 mL) then treated with 4.0 M HCl in 1,4-dioxane (170 μL, 0.68 mmol) for 24 h. The mixture was concentrated under reduced pressure to % of the initial volume and then diluted with H₂O (10 mL). The aqueous phase was washed with Et₂O (2×10 mL), EtOAc (2×10 mL), and DCM (2×10 mL). The acidic aqueous phase was basified by adding aq. NaHCO₃ ss and extracted with DCM/MeOH (8:2 v/v, 3×20 mL). The combined organic layers were dried over Na₂S04 and concentrated under reduced pressure. The title compound I-8 (18 mg, 0.05 mmol) was obtained as a brownish solid. Yield: 56%. ¹H-NMR (400 MHz, CD₃OD) δ 2.74-2-83 (m, 2H), 2.89-2.95 (m, 1H), 3.17-3.23 (m, 1H), 3.68-3.72 (m, 1H), 4.11-4.15 (m, 1H), 6.70-6.77 (m, 4H), 6.81-6.85 (m, 1H), 7.65 (t, J=7.7 Hz, 1H), 8.22 (d, J=8.22 Hz, 1H), 8.27-8.31 (m, 2H), 8.48 (d, J=6.2 Hz, 1H), 9.24 (s, 1H). UHPLC purity: ≥90%. MS-ESI(+) m/z: 354.4 (M+H); MS-ESI(−) m/z: 352.4 (M−H).

### Example 72: (±)-trans-N-(4-Phenylpyrrolidin-3-yl)isoquinoline-5-carboxamide (Compound I-9)

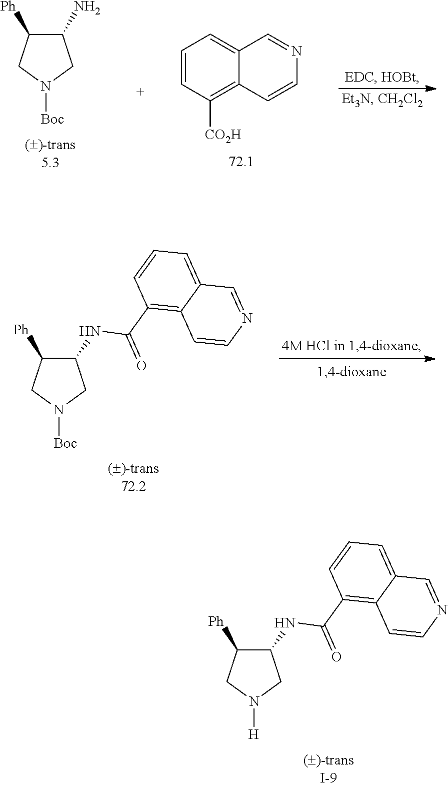

### Step 1: tert-Butyl (±)-trans-4-phenyl-3-[(isoquinolin-5-ylcarbonyl)amino]pyrrolidine-1-carboxylate(72.2)

To a stirred solution of intermediate 72.1 (99 mg, 0.57 mmol) in DCM (5 mL) cooled to 0° C., were added HOBt (77 mg, 0.57 mmol), EDC (109 mg, 0.57 mmol), and Et₃N (107 μL, 0.76 mmol) and the mixture was stirred for 45 min. A solution of intermediate 5.3 (100 mg, 0.38 mmol) in DCM (5 mL) was then added dropwise, and the reaction stirred at r.t. for 18 h. The mixture was washed with 0.5 M aq. citric acid (3×10 mL), H₂O (10 mL), aq. NaHCO₃ ss (10 mL), brine (10 mL), dried over Na₂SO₄, and concentrated under reduced pressure. The residue was purified by flash chromatography (DCM/MeOH from 98:2 to 90:10 v/v), to afford the intermediate 72.2 (98 mg, 0.23 mmol). Yield: 61%. MS-ESI(+) m/z: 418.5 (M+H); MS-ESI(−) m/z: 416.6 (M−H).

### Step 2: (±)-trans-N-(4-Phenylpyrrolidin-3-yl)isoquinoline-5-carboxamide (Compound I-9)

Compound I-9 was synthesized starting from intermediate 72.2 (98 mg, 0.23 mmol) which was reacted with 4.0 M HCl in 1,4-dioxane (0.41 mL, 1.64 mmol), in 1,4-dioxane (3 mL). After work-up, the title compound I-9 (58 mg, 0.18 mmol) was obtained as an orange powder. Yield: 78%. ¹H-NMR (400 MHz, CD₃OD) δ 2.94-2.99 (m, 1H), 3.06-3.11 (m, 1H), 3.32-3.36 (m, 1H), 3.44-3.57 (m, 3H), 7.29-7.32 (m, 1H), 7.37-7.44 (m, 4H), 7.71 (t, J=7.8 Hz, 1H), 7.80 (d, J=6.1 Hz, 1H), 7.86 (d, J=7.1 Hz, 1H), 8.21 (d, J=8.2 Hz, 1H), 8.39 (d, J=6.0 Hz, 1H), 9.27 (s, 1H). UHPLC purity: ≥95%. MS-ESI(+) m/z: 318.5 (M+H); MS-ESI(−) m/z: 316.4 (M−H).

### Example 73: (±)-trans-N-[4-Phenylpyrrolidin-3-yl][1,3]thiazolo[4,5-c]pyridin-2-amine hydrochloride (I-10)

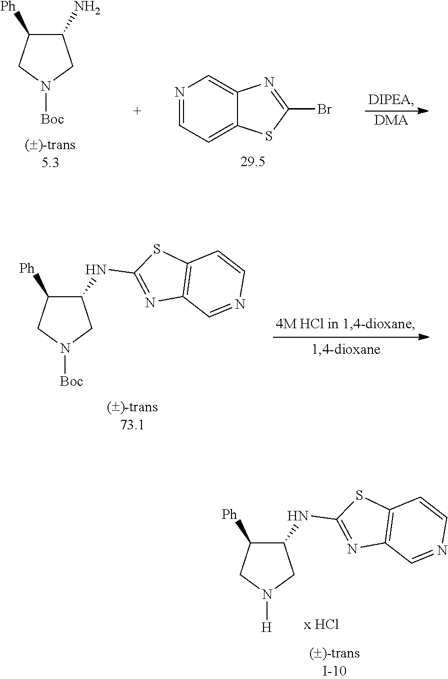

### Step 1: tert-Butyl (±)-trans-4-phenyl-3-([1,3]thiazolo[4,5-c]pyridin-2-ylamino)pyrrolidine-1-carboxylate (73.1)

DIPEA (44 μL, 0.25 mmol), and intermediate 29.5 (27 mg, 0.13 mmol) were sequentially added to a stirred solution of intermediate 5.3 (40 mg, 0.15 mmol) in DMA (0.5 mL) under a N₂ atmosphere, and the resulting mixture was reacted at 100° C. for 2 days. The mixture was poured into H₂O (20 mL) and extracted with EtOAc (3×10 mL). The combined organic layers were washed with H₂O (20 mL) and brine (20 mL), dried over anhydrous Na₂SO₄, and evaporated to dryness. The residue was purified by flash chromatography (DCM/MeOH, from 100% DCM, to 95:5 v/v DCM/MeOH) to provide the desired intermediate 73.1 (36 mg, 0.09 mmol). Yield: 69%. MS-ESI(+) m/z: 397.2 (M+H); MS-ESI(−) m/z: 395.1 (M−H).

### Step 2: (±)-trans-N-[4-Phenylpyrrolidin-3-yl][1,3]thiazolo[4,5-c]pyridin-2-amine hydrochloride (I-10)

To a stirred solution of intermediate 73.1 (36 mg, 0.09 mmol) in dry 1,4-dioxane (0.5 mL), was added 4.0M HCl in 1,4-dioxane (227 μL, 0.90 mmol), and the mixture was stirred at r.t. for 16 h. The resulting suspension was centrifuged, the supernatant was removed, and the residue was washed with Et₂O (2×1 mL). Upon centrifugation and desiccation in a drying oven, the title compound I-10 (15 mg, 0.05 mmol) was obtained as a yellow solid. Yield: 56%. ¹H-NMR (400 MHz, DMSO-d₆) δ 3.16-3.27 (m, 1H), 3.36-3.42 (m, 1H), 3.60-3.78 (m, 3H), 3.95-4.01 (m, 1H), 7.26-7.50 (m, 6H), 8.80 (brs, 2H), 9.85-10.09 (m, 2H). UHPLC purity: 90-95%. MS-ESI(+) m/z: 295.2 (M+H); MS-ESI(−) m/z: 297.2 (M−H).

### Example 74: (±)-trans-4-Phenyl-N-[3-(pyridin-3-yl)phenyl]pyrrolidine-3-carboxamide (Compound I-11)

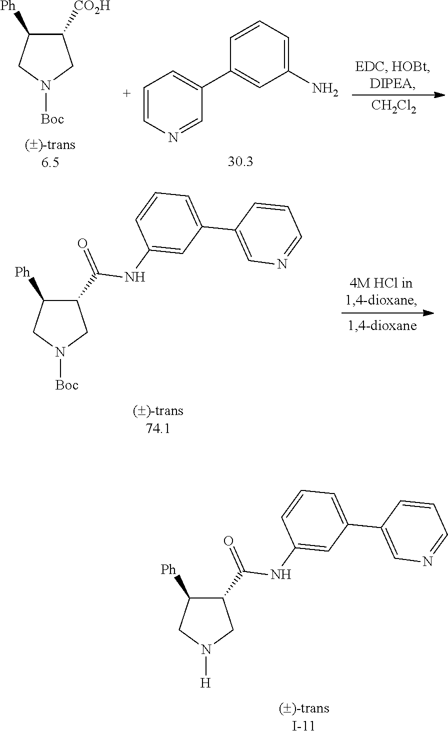

### Step 1: tert-Butyl (±)-trans-4-phenyl-3-{[3-(pyridin-3-yl)phenyl]carbamoyl}pyrrolidine-1-carboxylate (74.1)

To a solution of intermediate 6.5 (150 mg, 0.51 mmol) in DCM (15 mL) DIPEA (0.13 mL, 0.77 mmol), EDC (148 mg, 0.62 mmol), and HOBt (104 mg, 0.77 mmol) were added. Stirring was continued at r.t. for 15 min, after which intermediate 30.3 (105 mg, 0.62 mmol) was added. Stirring was then continued at r.t. for 16 h. The crude was poured into water (30 mL) and was washed with a 0.5 M solution of citric acid. The collected organic layers were washed with brine (20 mL), dried over Na₂SO₄, and concentrated under reduced pressure. The crude was purified by flash chromatography (DCM/MeOH from 0% to 4%) to give the intermediate 74.1 (90 mg, 0.2 mmol) as a colorless oil. Yield: 40%.

### Step 2: (±)-trans-4-Phenyl-N-[3-(pyridin-3-yl)phenyl]pyrrolidine-3-carboxamide (I-11)

To a solution of intermediate 74.1 (86 mg, 0.19 mmol) in 1,4-dioxane (5 mL) was added a 4M solution of HCl in 1,4-dioxane (0.5 mL, 1.93 mmol). Stirring was continued at rt 16 h and the solvent was removed under vacuum. The crude was taken up with H₂O (30 mL) and the pH was basified with NaHCO₃(ss). The aqueous phase was extracted with EtOAc (3×20 mL). The collected organic layers were washed with brine (20 mL), dried over Na₂SO₄ and concentrated under reduced pressure to give the title compound I-11 (30 mg, 0.08 mmol) as a yellowish solid. Yield: 40%. ¹H-NMR (400 MHz, CDCl₃) δ 3.04-3.06 (m, 2H), 3.04 (m, 1H), 3.53-3.68 (m, 3H), 7.29-7.45 (m, 10H), 7.77 (s, 1H); 7.86 (d, J=4.1 Hz, 1H), 8.04 (s, 1H), 8.55 (d, J=4.6 Hz, 1H), 8.8 (s, 1H). HPLC purity: 99.9%. MS-ESI(+) m/z: 344.1 (M+H);

### Example 75: (±)-trans-N-(Isoquinolin-1-yl)-4-phenylpyrrolidine-3-carboxamide dihydrochloride (Compound I-12)

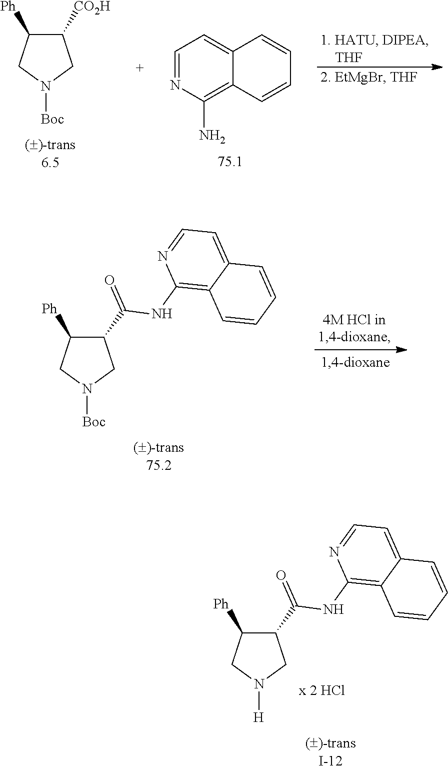

### Step 1: tert-Butyl (±)-trans-4-phenyl-3-(isoquinolin-1-ylcarbamoyl)pyrrolidine-1-carboxylate (75.2)

Intermediate 75.2 was synthesized according the procedure of Step 1 Example 64 starting from intermediate 6.5 (70 mg, 0.20 mmol) which was reacted with HATU (93 mg, 0.28 mmol), DIPEA (126 μL, 0.72 mmol), intermediate 50.1 (52 mg, 0.361 mmol), and 3.0 M EtMgBr in Et₂O (241 μL, 0.36 mmol) in THF (2 mL+2 mL). The intermediate 75.2 (40 mg, 0.10 mmol) was obtained after work-up and flash chromatography (DCM/MeOH, from 100% DCM, to 95:5 v/v DCM/MeOH). Yield: 40%. MS-ESI(+) m/z: 418.3 (M+H); MS-ESI(−) m/z: 416.3 (M−H).

### Step 2: (±)-trans-N-(Isoquinolin-1-yl)-4-phenylpyrrolidine-3-carboxamide dihydrochloride (Compound I-12)

Compound I-12 was synthesized according to the procedure described in Step 2 of Example 64 starting from a solution of intermediate 75.2 (40 mg, 0.10 mmol) in 1,4-dioxane (0.9 mL) with 4.0 M HCl in 1,4-dioxane (0.24 mL, 0.96 mmol). The title compound I-12 (40 mg, 0.10 mmol) was obtained as a brownish solid. Yield: quantitative. ¹H-NMR (400 MHz, DMSO-d₆) δ 3.24-3.30 (m, 1H), 3.33-3.42 (m, 1H), 3.56-3.67 (m, 2H), 3.68-3.85 (m, 2H), 7.25-7.32 (m, 4H), 7.38 (d, J=7.3 Hz, 2H), 7.83-7.86 (m, 2H), 7.91-7.94 (m, 1H), 8.34 (d, J=7.9 Hz, 1H), 8.78 (s, 1H), 9.49 (s, 1H), 9.60 (brs, 1H), 9.92 (brs, 1H), 10.81 (s, 1H). UHPLC purity: ≥95%. MS-ESI(+) m/z: 318.4 (M+H); MS-ESI(−) m/z: 316.5 (M−H).

### Example 76: (±)-trans-N-(Biphenyl-3-yl)-4-phenylpyrrolidine-3-carboxamide (Compound I-13)

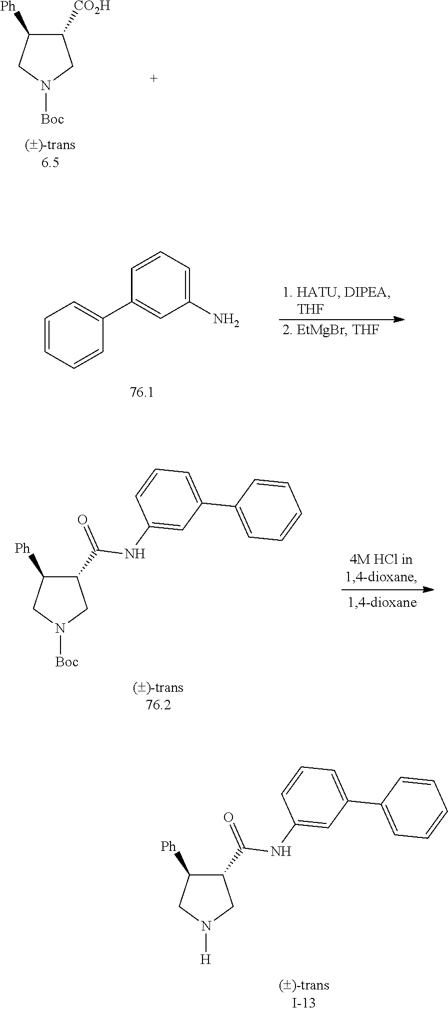 [SMILES: CC(C)(C)OC(=O)N1C[C@@H](C(=O)O)[C@H](c2ccccc2)C1 ; Nc1cccc(-c2ccccc2)c1 ; "2. EtMgBr, THF" ; "1. HATU, DIPEA,    THF" ; CC(C)(C)OC(=O)N1C[C@@H](C(=O)Nc2cccc(-c3ccccc3)c2)[C@H](c2ccccc2)C1 ; O=C(Nc1cccc(-c2ccccc2)c1)[C@@H]1CNC[C@H]1c1ccccc1]

### Step 1: tert-Butyl (±)-trans-4-phenyl-3-[(biphenyl-3-yl)carbamoyl]pyrrolidine-1-carboxylate (76.2)

DIPEA (0.27 mL, 1.53 mmol) and HATU (235 mg, 0.62 mmol) were added to a solution of intermediate 6.5 (150 mg, 0.51 mmol) in THF (5 mL). Stirring was continued at r.t. 1 h. In a separate flask, a 3.0 M solution of EtMgBr in Et₂O (0.51 mL, 1.53 mmol) was added to a solution of intermediate 76.1 (129.4 mg, 0.77 mmol) in THF (5 mL). After 10 minutes, the first solution was added via cannula to the second one and stirring was continued at r.t. for additional 4 h. The reaction was quenched by the addition of water (20 mL). The aqueous phase was extracted with EtOAc (3×20 mL). The collected organic layers were washed with brine (20 mL), dried over Na₂SO₄, and concentrated under reduced pressure. Purification by flash chromatography (PET/EtOAc from 100% PET to 80:20 v/v PET/EtOAc). The intermediate 76.2 (120 mg, 0.27 mmol) was obtained as a colorless oil. Yield 53%.

### Step 2: (±)-trans-N-(Biphenyl-3-yl)-4-phenylpyrrolidine-3-carboxamide (Compound I-13)

Intermediate 76.2 (120 mg, 0.27 mmol) was dissolved in 1,4-dioxane (5 mL) then a 4M solution of HCl in 1,4-dioxane (0.67 mL, 2.71 mmol) was added and stirring continued at r.t. for 16 h. The solvent was removed under vacuo. The crude was taken up with H₂O and the pH was basified with NaHCO₃(ss). The aqueous phase was extracted with EtOAc (3×20 mL). The collected organic layers were then washed with brine (20 mL), dried over Na₂SO₄, and concentrated under reduced pressure. Reverse phase flash chromatography purification (H₂O/MeCN from 100 to 20:80, v/v) afforded the title compound I-13 (30 mg, 0.087 mmol) as a white solid. Yield: 32%. ¹H-NMR (400 MHz, CDCl₃) δ 2.58 (brs, 2H), 2.98-3.02 (m, 2H), 3.39 (m, 1H), 3.54-3.61 (m, 3H), 7.27-7.38 (m, 8H), 7.41-7.45 (m, 2H), 7.41-7.45 (m, 2H), 7.57 (d, J=7.8 Hz, 2H), 7.67 (s, 1H), 7.72 (s, 1H). UHPLC purity: ≥95%. MS-ESI(+) m/z: 343.5 (M+H); MS-ESI(−) m/z: 341.3 (M−H).

### Example 77: (±)-trans-N-(Isoquinolin-3-yl)-4-phenylpyrrolidine-3-carboxamide dihydrochloride (Compound I-14)

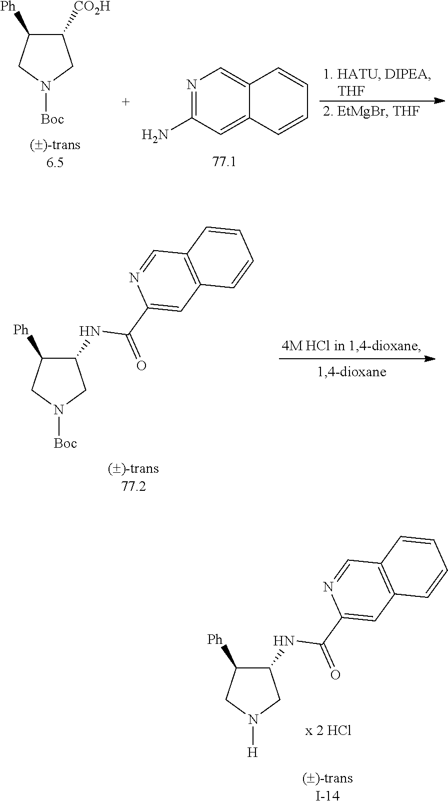

### Step 1: tert-Butyl (±)-trans-4-phenyl-3-(isoquinolin-3-ylcarbamoyl)pyrrolidine-1-carboxylate (77.2)

Compound 77.2 was synthesized according to the procedure described in Step 1 of Example 64 starting from intermediate 6.5 (70 mg, 0.20 mmol) which was reacted with HATU (110 mg, 0.29 mmol), DIPEA (126 μL, 0.72 mmol), intermediate 77.1 (52 mg, 0.36 mmol), and 3.0M EtMgBr in Et₂O (241 μL, 0.72 mmol) in THF (2 mL+2 mL). The intermediate 77.2 (63 mg, 0.15 mmol) was obtained after flash chromatography (DCM/MeOH, from 100% DCM, to 95:5 v/v DCM/MeOH). Yield: 75%. MS-ESI(+) m/z: 418.3 (M+H); MS-ESI(−) m/z: 416.3 (M−H).

### Step 2: (±)-trans-N-(Isoquinolin-3-yl)-4-phenylpyrrolidine-3-carboxamide dihydrochloride (Compound I-14)

Compound I-14 was synthesized according to the procedure described in Step 2 of Example 64 starting from a solution of intermediate 77.2 (63 mg, 0.15 mmol) in 1,4-dioxane (1 mL) which was reacted with 4.0 M HCl in 1,4-dioxane (0.37 mL, 0.96 mmol). The title Compound I-14 (52 mg, 0.13 mmol) was obtained as a yellowish solid. Yield: 87%. ¹H-NMR (400 MHz, DMSO-d₆) δ 3.13-3.18 (m, 1H), 3.27-3.29 (m, 1H), 3.56-3.70 (m, 4H), 7.18 (d, J=7.2 Hz, 1H), 7.24-7.32 (m, 4H), 7.44 (t, J=7.0 Hz, 1H), 7.62 (t, J=7.0 Hz, 1H), 7.80 (d, J=8.2 Hz, 1H), 7.95 (d, J=8.2 Hz, 1H), 8.36 (s, 1H), 9.02 (s, 1H), 9.68 (brs, 1H), 9.84 (brs, 1H), 10.90 (s, 1H). UHPLC purity: ≥95%. MS-ESI(+) m/z: 318.5 (M+H); MS-ESI(−) m/z: 316.5 (M−H).

### Example 78: (±)-trans-N-(3-Methylisoquinolin-5-yl)-4-phenylpyrrolidine-3-carboxamide dihydrochloride (Compound I-15)

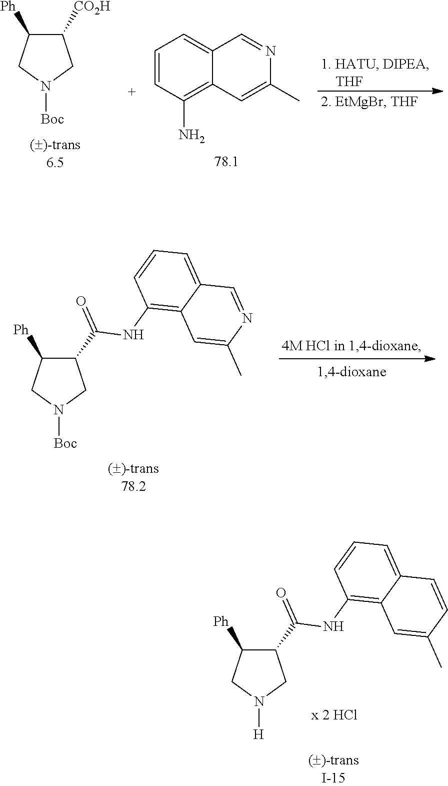

### Step 1: tert-Butyl (±)-trans-4-phenyl-3-[(3-methylisoquinolin-5-yl)carbamoyl]pyrrolidine-1-carboxylate (78.2)

Intermediate 78.2 was synthesized according to the procedure described in Step 1 of Example 64 starting from intermediate 6.5 (85 mg, 0.29 mmol) which was reacted with HATU (133 mg, 0.35 mmol), DIPEA (154 μL, 0.88 mmol), intermediate 78.1 (70 mg, 0.44 mmol), and 3.0 M EtMgBr in Et₂O (294 μL, 0.88 mmol) in THF (2 mL+2 mL). The intermediate 78.2 (23 mg, 0.05 mmol) was obtained after flash chromatography (DCM/MeOH, from 100% DCM, to 95:5 v/v DCM/MeOH). Yield: 17%. MS-ESI(+) m/z: 432.7 (M+H); MS-ESI(−) m/z: 430.8 (M−H).

### Step 2: (±)-trans-N-(3-Methylisoquinolin-5-yl)-4-phenylpyrrolidine-3-carboxamide dihydrochloride (Compound I-15)

Compound I-15 was synthesized according to the procedure described in Step 2 of Example 64 starting from a solution of intermediate 78.2 (23 mg, 0.05 mmol) in 1,4-dioxane (0.7 mL) which was reacted with 4.0 M HCl in 1,4-dioxane (133 μL, 0.53 mmol). The title compound I-15 (16 mg, 0.04 mmol) was obtained as a yellow solid. Yield: 80%. ¹H-NMR (400 MHz, DMSO-d₆) δ 3.27-3.66 (m, 6H), 7.27 (d, J=7.2 Hz, 1H), 7.32 (t, J=7.5 Hz, 2H), 7.39 (d, J=7.3 Hz, 2H), 7.53 (s, 1H), 7.72 (t, J=7.9 Hz, 1H), 8.00 (d, J=7.3 Hz, 1H), 8.12 (d, J=8.0 Hz, 1H), 9.60 (s, 1H), 9.70 (brs, 1H), 10.02 (brs, 1H), 10.58 (s, 1H). UHPLC purity: ≥95%. MS-ESI(+) m/z: 332.5 (M+H); MS-ESI(−) m/z: 330.5 (M−H).

### Example 79: (±)-trans-N-(Naphthalen-1-yl)-4-phenylpyrrolidine-3-carboxamide hydrochloride (Compound I-16)

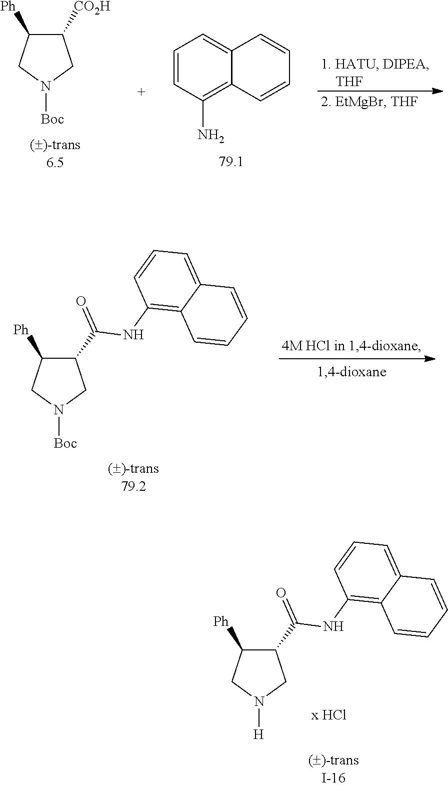

### Step 1: tert-Butyl (±)-trans-4-phenyl-3-(naphthalen-1-ylcarbamoyl)pyrrolidine-1-carboxylate (79.2)

Intermediate 79.2 was synthesized according to the procedure described in Step 1 of Example 64 starting from intermediate 6.5 (110 mg, 0.38 mmol) which was reacted with HATU (172 mg, 0.45 mmol), DIPEA (198 μL, 1.13 mmol), intermediate 79.1 (81 mg, 0.57 mmol), and 3.0 M EtMgBr in Et₂O (378 μL, 0.13 mmol) in THF (2 mL+2 mL). The intermediate 79.2 (31 mg, 0.07 mmol) was obtained after flash chromatography (DCM/MeOH, from 100% DCM, to 95:5 v/v DCM/MeOH). Yield: 18%. MS-ESI(−) m/z: 415.6 (M−H).

### Step 2: (±)-trans-N-(Naphthalen-1-yl)-4-phenylpyrrolidine-3-carboxamide dihydrochloride (Compound I-16)

Compound I-16 was synthesized according to the procedure described in Step 2 of Example 64 starting from a solution of intermediate 79.2 (31 mg, 0.07 mmol) in 1,4-dioxane (0.7 mL) which was reacted with 4.0 M HCl in 1,4-dioxane (186 μL, 0.74 mmol). The title compound I-16 (21 mg, 0.06 mmol) was obtained as a white solid. Yield: 86%. ¹H-NMR (400 MHz, DMSO-d₆) δ 3.47-3.59 (m, 4H), 3.61-3.65 (m, 1H), 3.66-3.72 (m, 1H), 7.26-7.43 (m, 9H), 7.67 (d, J=8.1 Hz, 1H), 7.80 (d, J=8.1 Hz, 1H), 9.42 (brs, 1H), 9.72 (brs, 1H), 10.06 (s, 1H). UHPLC purity: ≥95%. MS-ESI(+) m/z: 317.5 (M+H); MS-ESI(−) m/z: 315.4 (M−H).

### Example 80: (±)-trans-4-Phenyl-N-(quinolin-5-yl)pyrrolidine-3-carboxamide dihydrochloride (Compound I-17)

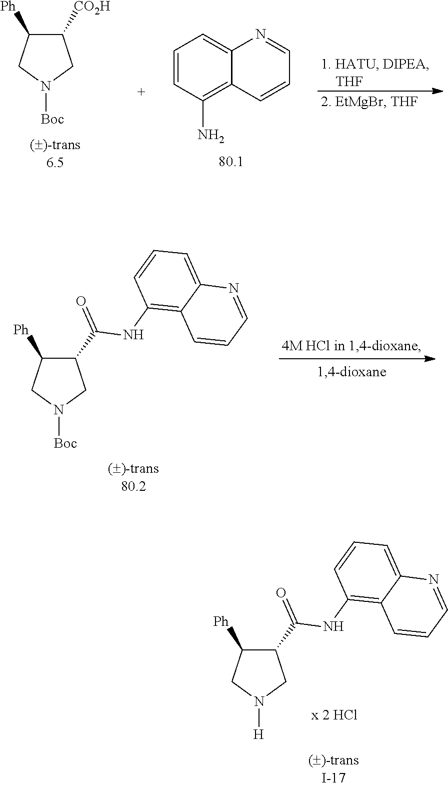

### Step 1: tert-Butyl (±)-trans-4-phenyl-3-(quinolin-5-ylcarbamoyl)pyrrolidine-1-carboxylate (80.2)

Intermediate 80.2 was synthesized according to the procedure described in Step 1 of Example 64 starting from intermediate 6.5 (70 mg, 0.24 mmol) which was reacted with HATU (110 mg, 0.29 mmol), DIPEA (126 μL, 0.72 mmol), intermediate 80.1 (52 mg, 0.36 mmol), and 3.0 M EtMgBr in Et₂O (241 μL, 0.72 mmol) in THF (2 mL+2 mL). The intermediate 80.2 (35 mg, 0.08 mmol) was obtained after flash chromatography (DCM/MeOH, from 100% DCM, to 95:5 v/v DCM/MeOH). Yield: 33%. MS-ESI(+) m/z: 418.7 (M+H); MS-ESI(−) m/z: 416.7 (M−H).

### Step 2: (±)-trans-4-Phenyl-N-(quinolin-5-yl)pyrrolidine-3-carboxamide dihydrochloride (Compound I-17)

Compound I-17 was synthesized according to the procedure described in Step 2 of Example 64 starting from a solution of intermediate 80.2 (35 mg, 0.08 mmol) in 1,4-dioxane (0.8 mL) which was reacted with 4.0 M HCl in 1,4-dioxane (210 μL, 0.84 mmol). The title compound I-17 (31 mg, 0.08 mmol) was obtained as a brown solid. Yield: quantitative. ¹H-NMR (400 MHz, DMSO-d₆) δ 3.24-3.39 (m, 2H), 3.56-3.67 (m, 4H), 7.27 (d, J=7.1 Hz, 1H), 7.32 (t, J=7.5 Hz, 2H), 7.38 (d, J=7.4 Hz, 2H), 7.73 (dd, J=5.0 Hz, J=8.0 Hz, 1H), 7.80 (d, J=7.4 Hz, 1H), 7.90 (t, J=8.0 Hz, 1H), 8.01 (d, J=8.7 Hz, 1H), 8.38 (d, J=8.2 Hz, 1H), 9.08 (d, J=4.9 Hz, 1H), 9.56 (brs, 1H), 9.88 (brs, 1H), 10.69 (s, 1H). UHPLC purity: ≥95%. MS-ESI(+) m/z: 318.6 (M+H); MS-ESI(−) m/z: 316.5 (M−H).

### Example 81: (±)-trans-4-Phenyl-N-(quinolin-8-yl)pyrrolidine-3-carboxamide dihydrochloride (Compound I-18)

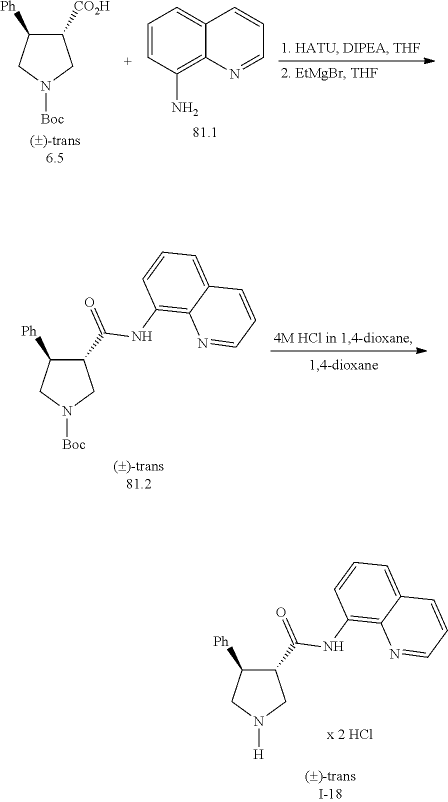 [SMILES: CC(C)(C)OC(=O)N1C[C@@H](C(=O)O)[C@H](c2ccccc2)C1 ; Nc1cccc2cccnc12 ; CC(C)(C)OC(=O)N1C[C@@H](C(=O)Nc2cccc3cccnc23)[C@H](c2ccccc2)C1 ; O=C(Nc1cccc2cccnc12)[C@@H]1CNC[C@H]1c1ccccc1]

### Step 1: tert-Butyl (±)-trans-3-phenyl-3-(quinolin-8-ylcarbamoyl)pyrrolidine-1-carboxylate (81.2)

Intermediate 81.2 was synthesized according to the procedure described in Step 1 of Example 64 starting from intermediate 6.5 (70 mg, 0.24 mmol) which was reacted with HATU (110 mg, 0.29 mmol), DIPEA (126 μL, 0.72 mmol), intermediate 81.1 (52 mg, 0.57 mmol), and 3.0 M EtMgBr in Et₂O (241 μL, 0.72 mmol) in THF (2 mL+2 mL). The intermediate 81.2 (65 mg, 0.16 mmol) was obtained after flash chromatography (DCM/MeOH, from 100% DCM, to 95:5 v/v DCM/MeOH). Yield: 67%. MS-ESI(+) m/z: 418.7 (M+H).

### Step 2: (±)-trans-4-Phenyl-N-(quinolin-8-yl)pyrrolidine-3-carboxamide dihydrochloride (Compound I-18)

Compound I-18 was synthesized according to the procedure described in Step 2 of Example 64 starting from a solution of intermediate 81.2 (65 mg, 0.16 mmol) in 1,4-dioxane (1 mL) which was reacted with 4.0 M HCl in 1,4-dioxane (389 μL, 1.56 mmol). The title compound I-18 (63 mg, 0.16 mmol) was obtained as a white solid. Yield: quantitative. ¹H-NMR (400 MHz, DMSO-d₆) δ 3.16-3.22 (m, 1H), 3.37-3.41 (m, 1H), 3.64-3.71 (m, 3H), 3.77-3.82 (m, 1H) 7.20 (d, J=7.4 Hz, 1H), 7.26 (t, J=7.5 Hz, 2H), 7.37 (d, J=7.3 Hz, 2H), 7.49 (t, J=7.9 Hz, 1H), 7.54 (dd, J=4.3 Hz, J=8.0 Hz, 1H), 7.62 (d, J=7.7 Hz, 1H), 8.35 (d, J=7.0 Hz, 1H), 8.42 (d, J=7.3 Hz, 1H), 8.73 (dd, J=1.4 Hz, J=4.2 Hz, 1H), 9.61 (brs, 1H), 9.87 (brs, 1H), 10.10 (s, 1H). UHPLC purity: ≥95%. MS-ESI(+) m/z: 318.5 (M+H).

### Example 82: (±)-trans-4-Phenyl-N-(pyridin-3-yl)pyrrolidine-3-carboxamide dihydrochloride (Compound I-19)

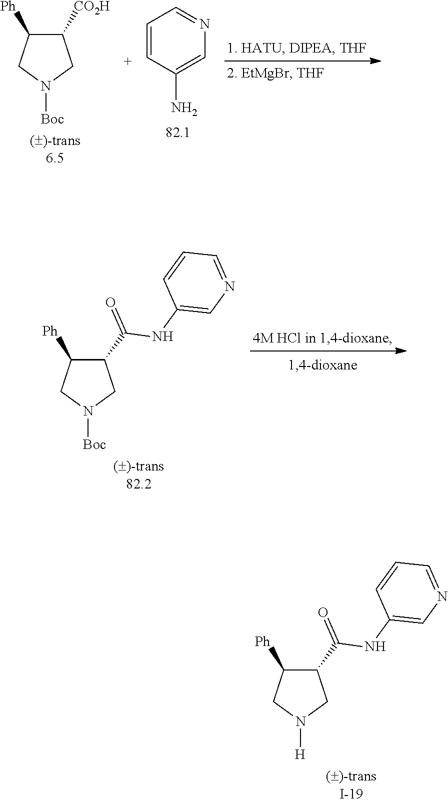 [SMILES: CC(C)(C)OC(=O)N1C[C@@H](C(=O)O)[C@H](c2ccccc2)C1 ; Nc1cccnc1 ; CC(C)(C)OC(=O)N1C[C@@H](C(=O)Nc2cccnc2)[C@H](c2ccccc2)C1 ; O=C(Nc1cccnc1)[C@@H]1CNC[C@H]1c1ccccc1]

### Step 1: tert-Butyl (±)-trans-4-phenyl-3-(pyridin-3-ylcarbamoyl)pyrrolidine-1-carboxylate (82.2)

Intermediate 82.2 was prepared according to the procedure described in Step 1 of Example 64 starting from a solution of intermediate 6.5 (150 mg, 0.51 mmol), HATU (235 mg, 0.62 mmol), DIPEA (0.27 mL, 1.53 mmol), 3.0 M EtMgBr in Et₂O (0.51 mL, 1.53 mmol), and intermediate 82.1 (72 mg, 0.77 mmol) in THF (5+5 mL). After purification by flash chromatography (DCM/MeOH from 100% DCM to 97:3 v/v DCM/MeOH) the intermediate 82.2 (150 mg, 0.41 mmol) was obtained as a colorless oil. Yield 80%.

### Step 2: (±)-trans-4-Phenyl-N-(pyridin-3-yl)pyrrolidine-3-carboxamide dihydrochloride (I-19)

Compound I-19 was prepared following the procedure described in Step 2 of Example 64 starting from a solution of intermediate 82.2 (167 mg, 0.45 mmol) in 1,4-dioxane (5 mL) and 4M HCl in 1,4-dioxane (1.13 mL, 4.54 mmol). Stirring was continued at r.t. for 16 h. The title compound I-19 (40 mg, 0.14 mmol) was obtained as a brownish solid. Yield: 33%. ¹H-NMR (400 MHz, DMSO-d₆) δ 2.92-2.97 (m, 1H), 3.09-3.18 (m, 2H), 3.43-3.51 (m, 3H), 3.54-3.59 (m, 1H), 7.21-7.24 (m, 1H), 7.29-7.34 (m, 5H), 8.01 (d, J=8.48 Hz, 1H), 8.24 (d, J=4.57 Hz, 1H), 8.69 (d, J=2.2 Hz, 1H), 10.28 (s, 1H). UHPLC purity: ≥93%. MS-ESI(+) m/z: 268.3 (M+H); MS-ESI(−) m/z: 266.3 (M−H).

### Example 83: (±)-trans-4-Phenyl-N-[5-(pyridin-3-yl)-1,3-thiazol-2-yl]pyrrolidine-3-carboxamide (Compound I-20)

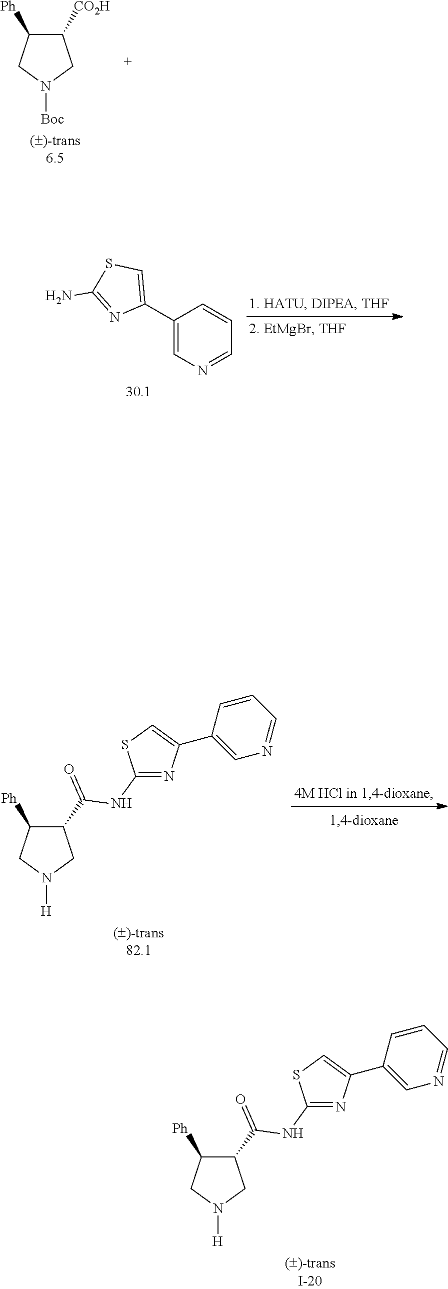 [SMILES: CC(C)(C)OC(=O)N1C[C@@H](C(=O)O)[C@H](c2ccccc2)C1 ; Nc1nc(-c2cccnc2)cs1 ; O=C(Nc1nc(-c2cccnc2)cs1)[C@@H]1CNC[C@H]1c1ccccc1 ; O=C(Nc1nc(-c2cccnc2)cs1)[C@@H]1CNC[C@H]1c1ccccc1]

### Step 1: tert-Butyl (±)-trans-4-phenyl-3-{[4-(pyridin-3-yl)-1,3-thiazol-2-yl]carbamoyl}pyrrolidine-1-carboxylate (82.1)

Intermediate 82.1 was prepared according to the procedure described in Step 1 of Example 64 starting from a solution of intermediate 6.5 (150 mg, 0.51 mmol), HATU (235 mg, 0.62 mmol), DIPEA (0.27 mL, 1.53 mmol), 3.0 M EtMgBr in Et₂O (0.51 mL, 1.53 mmol), and intermediate 30.1 (235 mg, 0.62 mmol) in THF (5 mL+5 mL). Stirring was continued at r.t. 16 h. The crude was poured into water, washed with a 0.5 M solution of citric acid and extracted with EtOAc (3×20 mL). The collected organic layers were washed with brine (20 mL), dried over Na₂SO₄, and concentrated under reduced pressure. After purification by flash chromatography (DCM/MeOH from 100% DCM to 97:3 v/v DCM/MeOH). The intermediate 82.1 (126 mg, 0.27 mmol) was obtained as a colorless oil. Yield 55%.

### Step 2: (±)-trans-4-Phenyl-N-[5-(pyridin-3-yl)-1,3-thiazol-2-yl]pyrrolidine-3-carboxamide (Compound I-20)

Compound I-20 was prepared following the procedure described in Step 2 of Example 64 starting from a solution of intermediate 82.1 (150 mg, 0.43 mmol) in 1,4-dioxane (5 mL) and 4M HCl in 1,4-dioxane (1.3 mL, 4.3 mmol). Stirring was continued at r.t. for 16 h. The title compound I-20 (60 mg, 0.17 mmol) as a white solid. Yield: 40%. ¹H-NMR (400 MHz, DMSO-d₆) δ 2.78 (dd, J=10.9 Hz, J=8.8 Hz, 1H), 3.02 (dd, J=10.6 Hz, J=6.7 Hz, 1H), 3.24-3.30 (m, 1H), 3.33-3.38 (m, 2H), 3.51-3.57 (m, 1H), 7.19-7.22 (m, 1H), 7.27-7.33 (m, 4H), 7.44 (dd, J=7.9 Hz, J=4.7 Hz, 1H), 7.79 (s, 1H), 8.18 (dt, J=8.52 Hz, J=1.7 Hz, 1H), 8.50 (dd, J=4.7 Hz, J=1.6 Hz), 9.0 (s, 1H). HPLC purity: ≥95%. MS-ESI(+) m/z: 351.4 (M+H); MS-ESI(−) m/z: 349.2 (M−H).

### Example 84: (±)-trans-N-(Isoquinolin-5-yl)-4-phenylpyrrolidine-3-carboxamide dihydrochloride (Compound I-21)

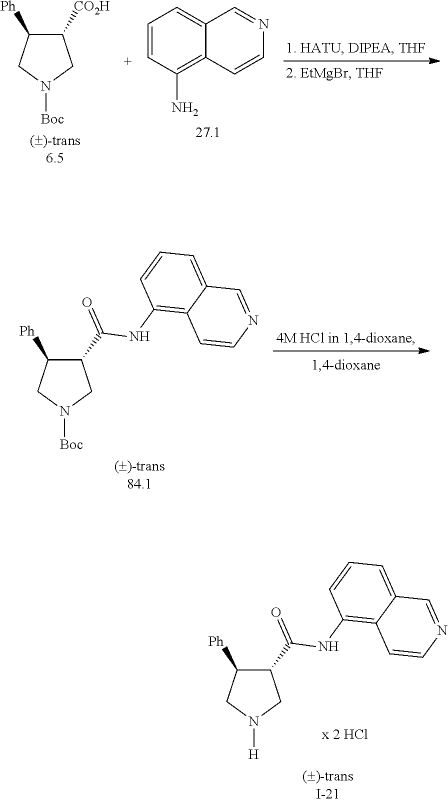 [SMILES: CC(C)(C)OC(=O)N1C[C@@H](C(=O)O)[C@H](c2ccccc2)C1 ; Nc1cccc2cnccc12 ; CC(C)(C)OC(=O)N1C[C@@H](C(=O)Nc2cccc3cnccc23)[C@H](c2ccccc2)C1 ; O=C(Nc1cccc2cnccc12)[C@@H]1CNC[C@H]1c1ccccc1]

### Step 1: tert-Butyl (±)-trans-4-phenyl-3-(isoquinolin-5-ylcarbamoyl)pyrrolidine-1-carboxylate (84.1)

Intermediate 84.1 was synthesized according to the procedure described in Step 1 of Example 64 starting from intermediate 6.5 (70 mg, 0.24 mmol), HATU (110 mg, 0.29 mmol), DIPEA (126 μL, 0.72 mmol), intermediate 27.1 (52 mg, 0.57 mmol), and 3.0 M EtMgBr in Et₂O (241 μL, 0.72 mmol) in THF (2 mL+2 mL). The intermediate 84.1 (17 mg, 0.04 mmol) was obtained after flash chromatography (DCM/MeOH, from 100% DCM, to 95:5 v/v DCM/MeOH). Yield: 17%. MS-ESI(+) m/z: 418.7 (M+H); MS-ESI(−) m/z: 416.7 (M−H).

### Step 2: (±)-trans-N-(Isoquinolin-5-yl)-4-phenylpyrrolidine-3-carboxamide dihydrochloride (Compound I-21)

Compound I-21 was synthesized according to the procedure described in Step 2 of Example 64 starting from a solution of intermediate 84.1 (17 mg, 0.04 mmol) in 1,4-dioxane (0.4 mL) which was reacted with 4.0 M HCl in 1,4-dioxane (102 μL, 0.41 mmol). The title compound I-21 (16 mg, 0.04 mmol) was obtained as an orange solid. Yield: quantitative. ¹H-NMR (400 MHz, DMSO-d₆) δ 3.26-3.37 (m, 1H), 3.39-3.49 (m, 1H), 3.66-3.77 (m, 3H), 3.78-3.90 (m, 1H), 7.36 (d, J=7.1 Hz, 1H), 7.41 (t, J=7.0 Hz, 2H), 7.47 (d, J=7.6 Hz, 2H), 7.91 (dd, J=8.0 Hz, J=6.8 Hz, 2H), 8.16 (d, J=7.5 Hz, 1H), 8.29 (d, J=8.1 Hz, 1H), 8.57 (d, J=6.6 Hz, 1H), 9.71 (brs, 1H), 9.80 (s, 1H), 10.03 (brs, 1H), 10.77 (s, 1H). UHPLC purity: ≥95%. MS-ESI(+) m/z: 318.6 (M+H); MS-ESI(−) m/z: 316.5 (M−H).

### Example 85: (±)-trans-N-(Biphenyl-3-yl)-4-(thiophen-2-yl)pyrrolidine-3-carboxamide hydrochloride (Compound I-22)

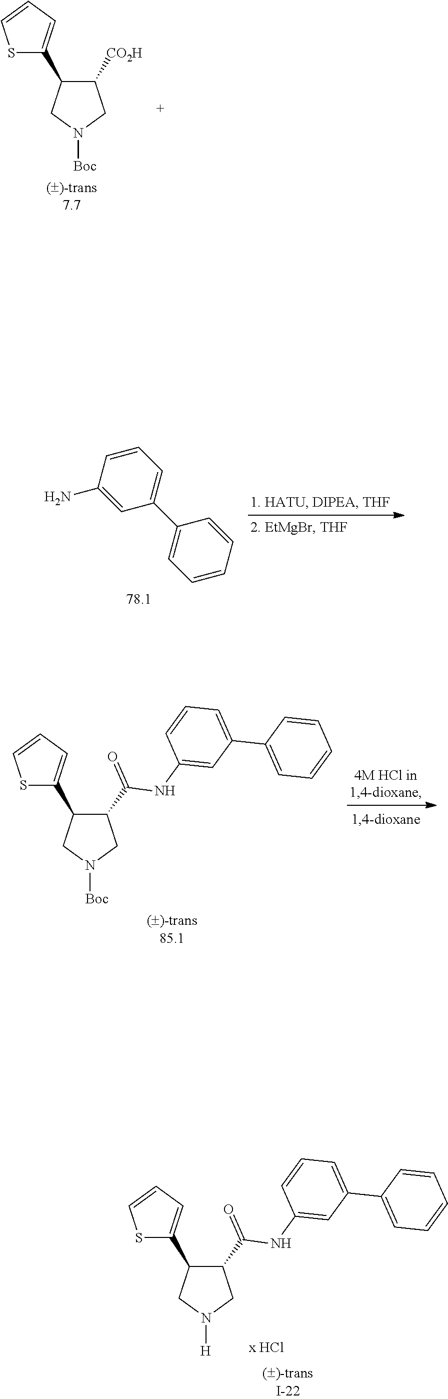 [SMILES: CC(C)(C)OC(=O)N1C[C@@H](C(=O)O)[C@H](c2cccs2)C1 ; Nc1cccc(-c2ccccc2)c1 ; CC(C)(C)OC(=O)N1C[C@@H](C(=O)Nc2cccc(-c3ccccc3)c2)[C@H](c2cccs2)C1 ; O=C(Nc1cccc(-c2ccccc2)c1)[C@@H]1CNC[C@H]1c1cccs1]

### Step 1: tert-Butyl (±)-trans-4-(thiophen-2-yl)-3-[(biphenyl-3-yl)carbamoyl]pyrrolidine-1-carboxylate (85.1)

Intermediate 85.1 was prepared according to the procedure described in Step 1 of Example 64 starting from a solution of intermediate 7.7 (150 mg, 0.51 mmol), HATU (230 mg, 0.61 mmol), DIPEA (0.26 mL, 1.5 mmol), 3.0 M EtMgBr in Et₂O (0.51 mL, 1.53 mmol), and intermediate 78.1 (101.5 mg, 0.6 mmol) in THF (5 mL+5 mL). Stirring was continued at r.t. for 16 h. Purification by flash chromatography (PET/EtOAc from 100% PET to 80:20 v/v PET/EtOAc). The intermediate 85.1 (80 mg, 0.17 mmol) was obtained as a colorless oil. Yield 36%.

### Step 2: (±)-trans-N-(Biphenyl-3-yl)-4-(thiophen-2-yl)pyrrolidine-3-carboxamide hydrochloride (Compound I-22)

Compound I-22 was prepared following the procedure described in Step 2 of Example 64 starting from a solution of intermediate 85.1 (80 mg, 0.17 mmol) in 1,4-dioxane (5 mL) and 4M HCl in 1,4-dioxane (0.54 mL, 1.78 mmol). Stirring was continued at r.t. 16 h. The title compound I-22 (55 mg, 0.14 mmol) was obtained as a yellowish powder as hydrochloride salt, after trituration with Et₂O. Yield: 84%. ¹H-NMR (400 MHz, DMSO-d₆) δ 3.27-3.41 (m, 4H), 3.73-3.76 (m, 1H), 3.99-4.0 (m, 1H), 6.99 (t, J=3.6 Hz, 1H), 7.11 (d, J=2.6 Hz, 1H), 7.33-7.38 (m, 3H), 7.43-7.47 (m, 3H), 7.57 (d, J=7.4 Hz, 3H), 7.92 (s, 1H), 9.51 (brs, 1H), 9.90 (brs, 1H), 10.6 (s, 1H). HPLC purity: ≥95%. MS-ESI(+) m/z: 349.4 (M+H); MS-ESI(−) m/z: 347.3 (M−H).

### Example 86: (±)-trans-N-(Biphenyl-3-yl)-4-(4-fluorophenyl)pyrrolidine-3-carboxamide hydrochloride (Compound I-23)

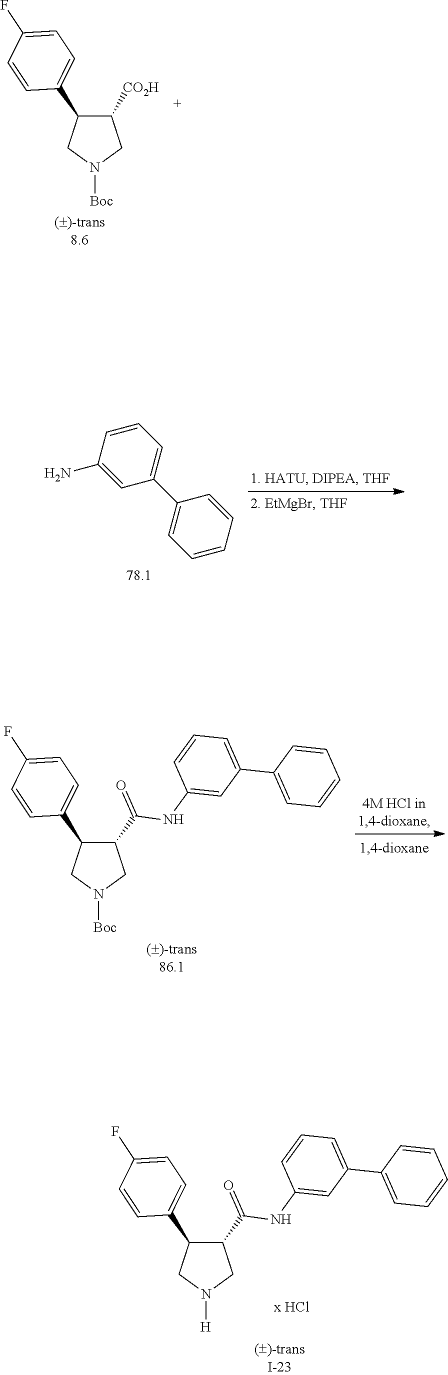 [SMILES: CC(C)(C)OC(=O)N1C[C@@H](C(=O)O)[C@H](c2ccc(F)cc2)C1 ; Nc1cccc(-c2ccccc2)c1 ; CC(C)(C)OC(=O)N1C[C@@H](C(=O)Nc2cccc(-c3ccccc3)c2)[C@H](c2ccc(F)cc2)C1 ; O=C(Nc1cccc(-c2ccccc2)c1)[C@@H]1CNC[C@H]1c1ccc(F)cc1]

### Step 1: tert-Butyl (±)-trans-4-(4-fluorophenyl)-3-[(biphenyl-3-yl)carbamoyl]pyrrolidine-1-carboxylate (86.1)

Intermediate 86.1 was prepared according to the procedure described in Step 1 of Example 64 starting from a solution of intermediate 8.6 (150 mg, 0.51 mmol), HATU (230 mg, 0.61 mmol), DIPEA (0.26 mL, 1.5 mmol), 3.0 M EtMgBr in Et₂O (0.51 mL, 1.53 mmol), and intermediate 78.1 (101.5 mg, 0.6 mmol) in THF (5 mL+5 mL). Stirring was continued at r.t. 16 h. Purification by flash chromatography (DCM/MeOH from 100% DCM to 97.5:2.5 v/v DCM/MeOH). The intermediate 86.1 (55 mg, 0.11 mmol) was obtained as a colorless oil. Yield 25%.

### Step 2: (±)-trans-N-(Biphenyl-3-yl)-4-(4-fluorophenyl)pyrrolidine-3-carboxamide hydrochloride (Compound I-23)

Compound I-23 was prepared following the procedure described in Step 2 of Example 64 starting from a solution of intermediate 86.1 (55 mg, 0.12 mmol) in 1,4-dioxane (5 mL), and 4M HCl in 1,4-dioxane (0.3 mL, 1.19 mmol). Stirring was continued at r.t. for 16 h. The title compound I-23 hydrochloride salt (20 mg, 0.05 mmol) was obtained as a yellowish powder after trituration with Et₂O. Yield: 42%. ¹H-NMR (400 MHz, DMSOd₆) δ 3.26-3.42 (m, 3H), 3.71-3.74 (m, 3H), 7.17 (t, J=8.62 Hz, 2H), 7.31-7.36 (m, 3H), 7.44-7.46 (m, 4H), 7.54 (t, J=7.8 Hz, 3H), 7.86 (s, 1H), 9.50 (brs, 1H), 9.90 (brs, 1H), 10.48 (s, 1H). Yield 42%. HPLC purity: ≥95%. MS-ESI(+) m/z: 361.4 (M+H); MS-ESI(−) m/z: 359.3 (M−H).

### Example 87: (±)-trans-N-(Biphenyl-4-yl)-4-phenylpyrrolidine-3-carboxamide hydrochloride (Compound I-24)

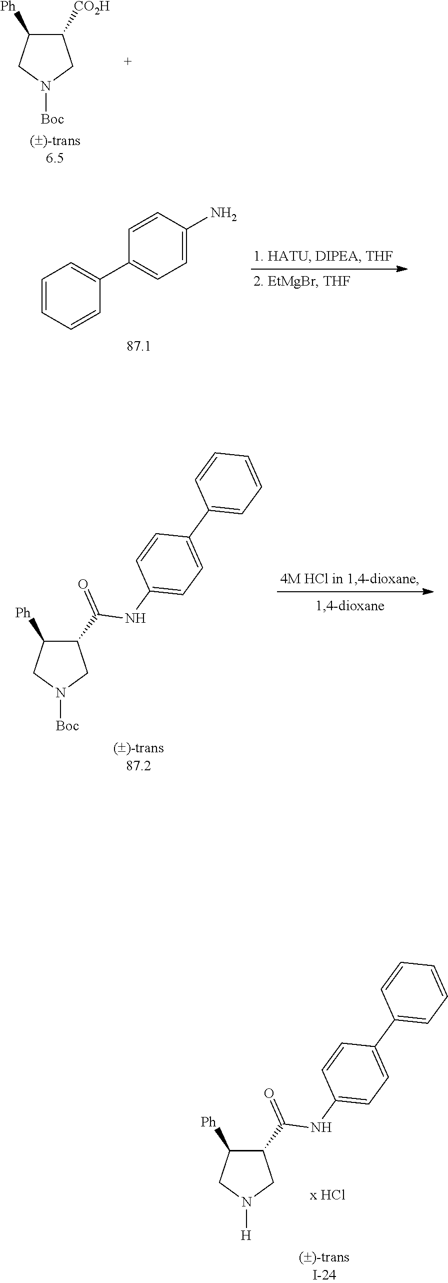 [SMILES: CC(C)(C)OC(=O)N1C[C@@H](C(=O)O)[C@H](c2ccccc2)C1 ; Nc1ccc(-c2ccccc2)cc1 ; CC(C)(C)OC(=O)N1C[C@@H](C(=O)Nc2ccc(-c3ccccc3)cc2)[C@H](c2ccccc2)C1 ; O=C(Nc1ccc(-c2ccccc2)cc1)[C@@H]1CNC[C@H]1c1ccccc1]

### Step 1: tert-Butyl (±)-trans-4-phenyl-3-[(biphenyl-4-yl)carbamoyl]pyrrolidine-1-carboxylate (87.2)

Intermediate 87.2 was prepared according to the procedure described in Step 1 of Example 64 starting from a solution of intermediate 6.5 (150 mg, 0.51 mmol), HATU (235 mg, 0.62 mmol), DIPEA (0.27 mL, 1.5 mmol), 3.0 M EtMgBr in Et₂O (0.51 mL, 1.53 mmol), and intermediate 87.1 (101.5 mg, 0.6 mmol) in THF (5 mL+5 mL). Stirring was continued at r.t. 16 h. Purification by flash chromatography (PET/EtOAc from 100% PET to 75:25 v/v PET/EtOAc). The intermediate 87.2 (120 mg, 0.27 mmol) was obtained as a white solid. Yield 53%.

### Step 2: (±)-trans-N-(Biphenyl-4-yl)-4-phenylpyrrolidine-3-carboxamide hydrochloride (Compound I-24)

Compound I-24 was prepared following the procedure described in Step 2 of Example 64 starting from a solution of intermediate 87.2 (120 mg, 0.27 mmol) in 1,4-dioxane (5 mL) and 4M HCl in 1,4-dioxane (0.67 mL, 2.71 mmol). Stirring was continued at r.t. for 16 h. The title compound I-24 (80 mg, 0.21 mmol) was obtained as a white powder as hydrochloride salt after trituration with Et₂O. Yield: 78%. ¹H-NMR (400 MHz, DMSO-d₆) δ 3.27-3.35 (m, 2H), 3.41-3.43 (m, 1H), 3.70-3.74 (m, 3H), 7.26-7.30 (m, 2H), 7.34 (t, J=7.2 Hz, 2H), 7.37-7.42 (m, 4H), 7.56-7.60 (m, 3H), 7.62-7.64 (m, 3H), 9.50 (bsr, 1H), 9.85 (brs, 1H), 10.43 (s, 1H). Yield 78%. HPLC purity: ≥95%. MS-ESI(+) m/z: 343.4 (M+H); MS-ESI(−) m/z: 341.4 (M−H).

### Example 88: (±)-trans-4-Phenyl-N-[4-(pyridin-3-yl)phenyl]pyrrolidine-3-carboxamide dihydrochloride (Compound I-25)

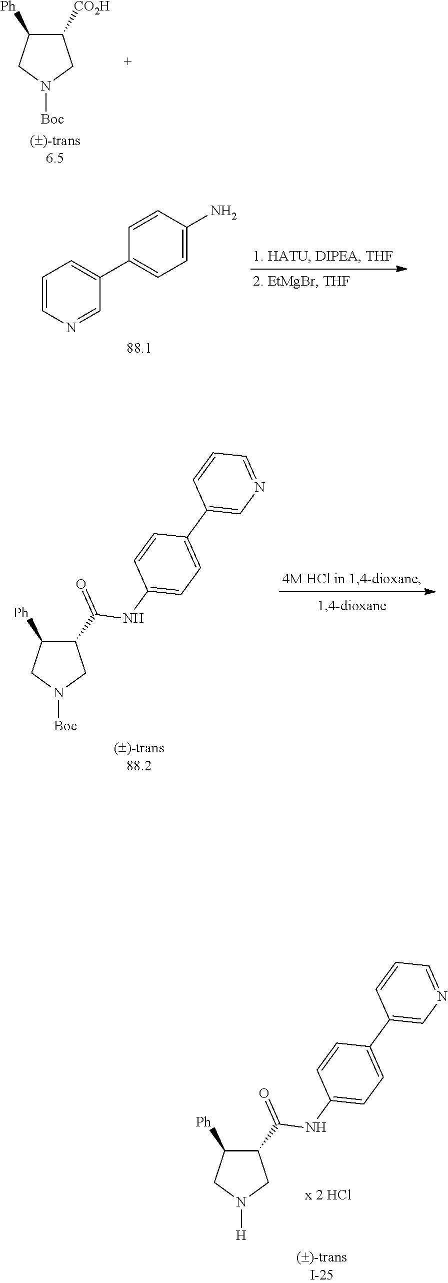 [SMILES: CC(C)(C)OC(=O)N1C[C@@H](C(=O)O)[C@H](c2ccccc2)C1 ; Nc1ccc(-c2cccnc2)cc1 ; CC(C)(C)OC(=O)N1C[C@@H](C(=O)Nc2ccc(-c3cccnc3)cc2)[C@H](c2ccccc2)C1 ; O=C(Nc1ccc(-c2cccnc2)cc1)[C@@H]1CNC[C@H]1c1ccccc1]

### Step 1: tert-Butyl (±)-trans-4-phenyl-3-{[4-(pyridin-3-yl)phenyl]carbamoyl}pyrrolidine-1-carboxylate (88.2)

Intermediate 88.2 was prepared according to the procedure described in Step 1 of Example 64 starting from a solution of intermediate 6.5 (94 mg, 0.32 mmol), HATU (144.3 mg, 0.38 mmol), DIPEA (0.17 mL, 0.96 mmol), 3.0 M EtMgBr in Et₂O (0.32 mL, 0.96 mmol), and intermediate 88.1 (70 mg, 0.41 mmol) in THF (5 mL+5 mL). Stirring was continued at r.t. 16 h. The crude was poured into H₂O, washed with 0.5 M solution of citric acid and extracted with EtOAc (3×20 mL). The collected organic layers were washed with brine (20 mL), dried over Na₂SO₄, and concentrated under reduced pressure. After purification by flash chromatography (DCM/MeOH from 100% DCM to DCM/MeOH 97.5:2.5, v/v) intermediate 88.2 (20 mg, 0.045 mmol) was obtained as a white solid. Yield 14%.

### Step 2: (±)-trans-4-Phenyl-N-[4-(pyridin-3-yl)phenyl]pyrrolidine-3-carboxamide dihydrochloride (Compound I-25)

Compound I-25 was prepared following the procedure described in Step 2 of Example 64 starting from a solution of intermediate 88.2 (60 mg, 0.13 mmol) in 1,4-dioxane (5 mL) and 4M HCl in 1,4-dioxane (0.33 mL, 1.3 mmol). Stirring was continued at r.t. for 16 h. The title compound I-25 (30 mg, 0.07 mmol) was obtained as a white powder as dihydrochloride salt after trituration with Et₂O. Yield: 55%. ¹H-NMR (400 MHz, DMSO-d₆) δ 3.25-3.34 (m, 2H), 3.48-3.53 (m, 2H), 3.71-3.74 (m, 3H), 7.6 (d, J=7.3 Hz, 1H), 7.33 (t, J=7.3 Hz, 2H), 7.38 (d, J=7.1 Hz, 2H), 7.74 (d, J=8.6 Hz, 2H), 7.80 (d, J=8.6 Hz, 2H), 7.96-7.99 (m, 1H), 8.69 (d, J=9.2 Hz, 1H), 8.77 (d, J=5.2 Hz, 1H), 9.14 (s, 1H), 9.70 (brs, 1H), 10.12 (brs, 1H). HPLC purity: 90%. MS-ESI(+) m/z: 344.4 (M+H); MS-ESI(−) m/z: 342.4 (M−H).

### Example 89: (±)-trans-N-[3-(6-Fluoropyridin-3-yl)phenyl]-4-phenylpyrrolidine-3-carboxamide (Compound I-26)

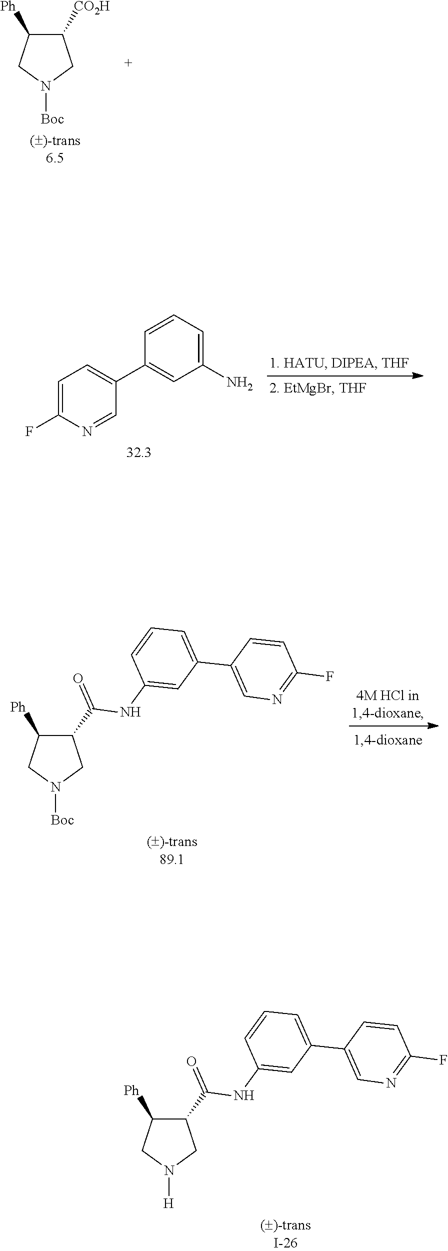 [SMILES: CC(C)(C)OC(=O)N1C[C@@H](C(=O)O)[C@H](c2ccccc2)C1 ; Nc1cccc(-c2ccc(F)nc2)c1 ; CC(C)(C)OC(=O)N1C[C@@H](C(=O)Nc2cccc(-c3ccc(F)nc3)c2)[C@H](c2ccccc2)C1 ; O=C(Nc1cccc(-c2ccc(F)nc2)c1)[C@@H]1CNC[C@H]1c1ccccc1]

### Step 1: tert-Butyl (±)-trans-4-phenyl-3-{[3-(6-fluoropyridin-3-yl)phenyl]carbamoyl}pyrrolidine-1-carboxylate (89.1)

Intermediate 89.1 was prepared according to the procedure described in Step 1 of Example 64 starting from a solution of intermediate 6.5 (150 mg, 0.51 mmol), HATU (235 mg, 0.62 mmol), DIPEA (0.27 mL, 1.53 mmol), 3.0 M EtMgBr in Et₂O (0.76 mL, 1.53 mmol), and intermediate 32.3 (145 mg, 0.77 mmol) in THF (5 mL+5 mL). Stirring was continued at r.t. for 16 h. The crude was poured into H₂O, washed with 0.5 M solution of citric acid and extracted with EtOAc (3×20 mL). The collected organic layers were washed with brine (20 mL), dried over Na₂SO₄, and concentrated under reduced pressure. After purification by flash chromatography (DCM/MeOH from 100% DCM to 97.5:2.5 v/v of DCM/MeOH) the intermediate 89.1 (120 mg, 0.04 mmol) was obtained as a colorless oil. Yield 51%.

### Step 2: (±)-trans-N-[3-(6-Fluoropyridin-3-yl)phenyl]-4-phenylpyrrolidine-3-carboxamide (Compound I-26)

Compound I-26 was prepared following the procedure described in Step 2 of Example 64 starting from a solution of intermediate 89.1 (100 mg, 0.33 mmol) in 1,4-dioxane (5 mL) and 4M HCl in 1,4-dioxane (0.55 mL, 2.2 mmol). Stirring was continued at r.t. for 16 h. After purification on reverse phase chromatography (H₂O/MeCN from 100% H₂O to 20:80 v/v H₂O/MeCN) the title compound I-26 (25 mg, 0.07 mmol) was obtained as a yellowish powder Yield: 31%. ¹H-NMR (400 MHz, CDCl₃) δ 3.13-3.18 (m, 2H), 3.57-3.64 (m, 4H), 6.94 (dd, J=8.45 Hz, J=2.9 Hz, 1H), 7.20-7.25 (m, 2H), 7.30-7.39 (m, 7H), 7.75 (s, 1H), 7.89 (dt, J=8.1 Hz, J=2.2 Hz, 1H), 8.31 (brs, 1H), 8.33 (s, 1H). HPLC purity: 90%. MS-ESI(+) m/z: 362.5 (M+H); MS-ESI(−) m/z: 360.3 (M−H).

### Example 90: (±)-trans-N-(Biphenyl-3-yl)-4-(3-fluorophenyl)pyrrolidine-3-carboxamide hydrochloride (Compound I-27)

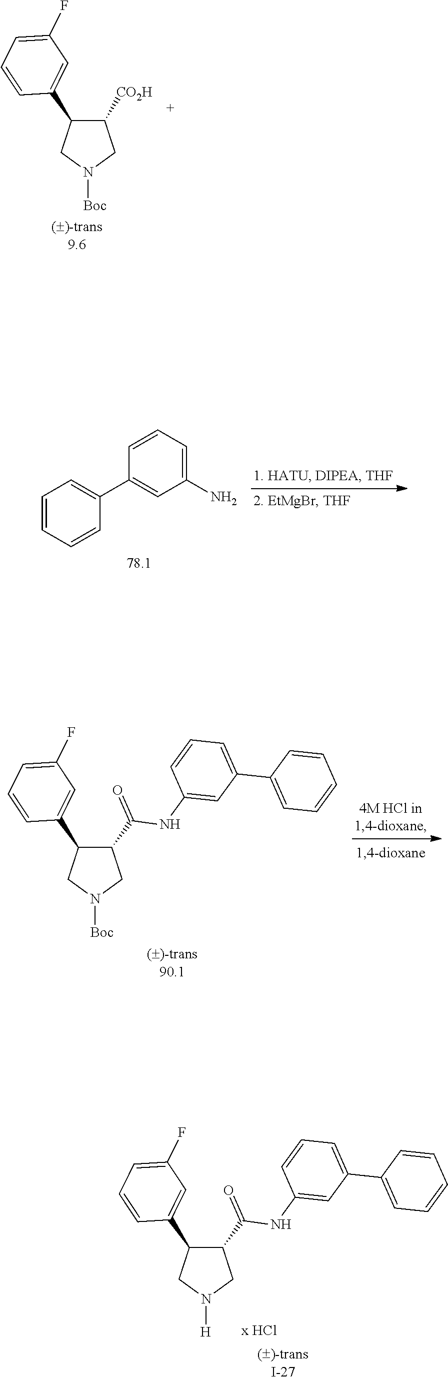 [SMILES: CC(C)(C)OC(=O)N1C[C@@H](C(=O)O)[C@H](c2cccc(F)c2)C1 ; Nc1cccc(-c2ccccc2)c1 ; CC(C)(C)OC(=O)N1C[C@@H](C(=O)Nc2cccc(-c3ccccc3)c2)[C@H](c2cccc(F)c2)C1 ; O=C(Nc1cccc(-c2ccccc2)c1)[C@@H]1CNC[C@H]1c1cccc(F)c1]

### Step 1: tert-Butyl (±)-trans-4-(3-fluorophenyl)-3-[(biphenyl-3-yl)carbamoyl]pyrrolidine-1-carboxylate (90.1)

Intermediate 90.1 was prepared according to the procedure described in Step 1 of Example 64 starting from a solution of intermediate 9.6 (187 mg, 0.6 mmol), HATU (273 mg, 0.72 mmol), DIPEA (0.31 mL, 1.8 mmol), 3.0 M EtMgBr in Et₂O (0.6 mL, 1.8 mmol), and intermediate 78.1 (132 mg, 0.78 mmol) in THF (5 mL+5 mL). Stirring was continued at r.t. for 16 h. After purification by flash chromatography (PET/EtOAc from 100% PET to 80:20 v/v PET/EtOAc) the intermediate 90.1 (120 mg, 0.04 mmol) was obtained as a yellowish powder. Yield 55%.

### Step 2: (±)-trans-N-(Biphenyl-3-yl)-4-(3-fluorophenyl)pyrrolidine-3-carboxamide hydrochloride (Compound I-27)

Compound I-27 was prepared following the procedure described in Step 2 of Example 64 starting from a solution of intermediate 90.1 (102 mg, 0.22 mmol) in 1,4-dioxane (5 mL) and 4M HCl in 1,4-dioxane (0.55 mL, 2.2 mmol). Stirring was continued at r.t. for 16 h. The title compound I-27 (50 mg, 0.125 mmol) was obtained as a yellowish powder. Yield: 57%. ¹H-NMR (400 MHz, CDCl₃) δ 3.31-3.45 (m, 3H), 3.73 (s, 3H), 7.09-7.20 (m, 2H), 7.32-7.53 (m, 10H), 7.85 (s, 1H), 9.55 (brs, 1H), 9.94 (brs, 1H), 10.5 (s, 1H). HPLC purity: ≥95%. MS-ESI(+) m/z: 361.4 (M+H); MS-ESI(−) m/z: 359.4 (M−H).

### Example 91: (±)-trans-4-(2-Fluorophenyl)-pyrrolidine-3-carboxylic acid biphenyl-3-ylamide hydrochloride (Compound I-28)

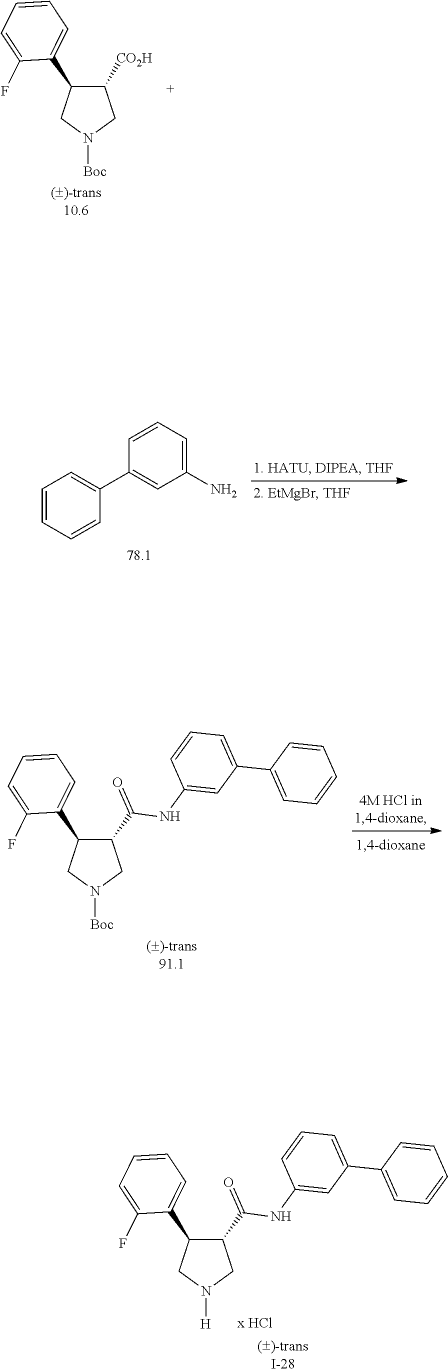 [SMILES: CC(C)(C)OC(=O)N1C[C@@H](C(=O)O)[C@H](c2ccccc2F)C1 ; Nc1cccc(-c2ccccc2)c1 ; CC(C)(C)OC(=O)N1C[C@@H](C(=O)Nc2cccc(-c3ccccc3)c2)[C@H](c2ccccc2F)C1 ; O=C(Nc1cccc(-c2ccccc2)c1)[C@@H]1CNC[C@H]1c1ccccc1F]

### Step 1: tert-Butyl (±)-trans-4-(2-fluorophenyl)-3-[(biphenyl-3-yl)carbamoyl]pyrrolidine-1-carboxylate (91.1)

Intermediate 91.1 was prepared according to the procedure described in Step 1 of Example 64 starting from a solution of intermediate 10.6 (175 mg, 0.57 mmol), HATU (258 mg, 0.68 mmol), DIPEA (0.3 mL, 1.71 mmol), 3.0 M EtMgBr in Et₂O (0.6 mL, 1.71 mmol), and intermediate 78.1 (115 mg, 0.68 mmol) in THF (5 mL+5 mL). Stirring was continued at r.t. for 16 h. Purification by flash chromatography (PET/EtOAc from 100% PET to 80:20 v/v PET/EtOAc). The title intermediate 91.1 (65 mg, 0.14 mmol) was obtained as a white powder. Yield 25%.

### Step 2: (±)-trans-4-(2-Fluorophenyl)-pyrrolidine-3-carboxylic acid biphenyl-3-ylamide hydrochloride (Compound I-28)

Compound I-28 was prepared following the procedure described in Step 2 of Example 64 starting from a solution of intermediate 91.1 (60 mg, 0.22 mmol) in 1,4-dioxane (5 mL) and 4M HCl in 1,4-dioxane (0.33 mL, 1.3 mmol). Stirring was continued at r.t. for 16 h. The title compound I-28 (45 mg, 0.11 mmol) was obtained as a grey powder Yield: 87%. ¹H-NMR (400 MHz, DMSO-d₆) δ 3.2 (m, 1H), 3.35-3.38 (m, 2H), 3.71-3.75 (m, 3H), 7.15-7.23 (m, 2H), 7.30-7.36 (m, 4H), 7.44 (t, J=7.6 Hz, 2H), 7.53 (t, J=7.6 Hz, 2H), 7.59 (t, J=7.5 Hz, 1H), 7.85 (s, 1H), 9.58 (brs, 1H), 9.97 (brs, 1H), 10.5 (s, 1H). HPLC purity: 92%. MS-ESI(+) m/z: 361.4 (M+H); MS-ESI(−) m/z: 359.3 (M−H).

### Example 92: (3R,4S)—N-(Isoquinolin-5-yl)-4-phenylpyrrolidine-3-carboxamide (Compound I-29)

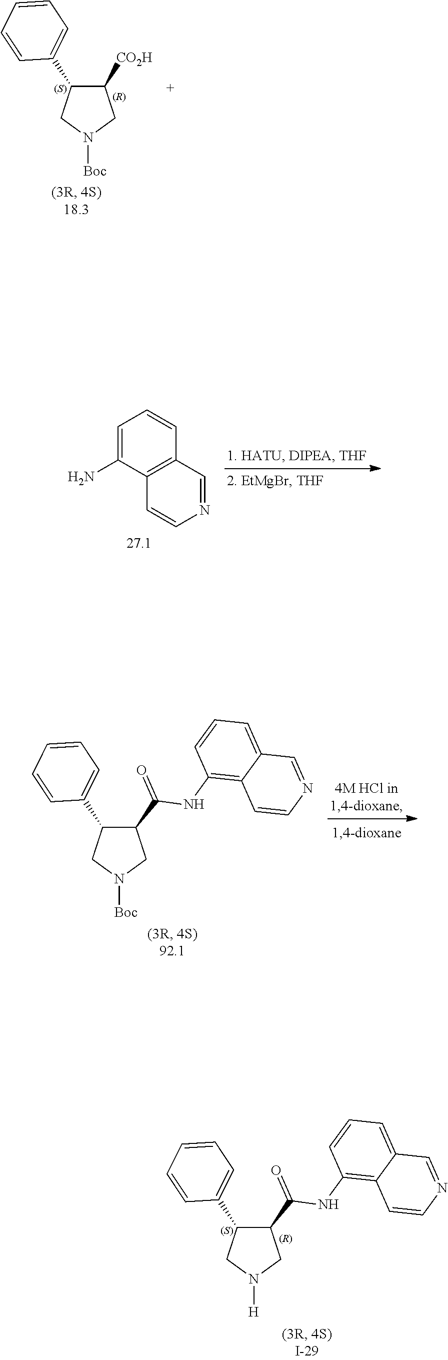

### Step 1: tert-Butyl (±)-trans-4-phenyl-3-(isoquinolin-5-ylcarbamoyl)pyrrolidine-1-carboxylate (92.1)

Intermediate 92.1 was synthesized according to the procedure described in Step 1 of Example 64 starting from intermediate 18.3 (70 mg, 0.24 mmol), HATU (110 mg, 0.29 mmol), DIPEA (126 μL, 0.72 mmol), intermediate 27.1 (52 mg, 0.57 mmol), and 3.0 M EtMgBr in Et₂O (241 μL, 0.72 mmol) in THF (2 mL+2 mL). The intermediate 92.1 was obtained after flash chromatography (DCM/MeOH, from 100% DCM, to 95:5 v/v DCM/MeOH) (46 mg, 0.11 mmol). Yield: 46%. MS-ESI(+) m/z: 418.3 (M+H); MS-ESI(−) m/z: 416.3 (M−H).

### Step 2: (3R,4S)—N-(Isoquinolin-5-yl)-4-phenylpyrrolidine-3-carboxamide (Compound I-29)

Compound I-29 was synthesized according to the procedure described in Step 2 of Example 64 starting from a solution of intermediate 92.1 (46 mg, 0.11 mmol) in 1,4-dioxane (0.8 mL) which was reacted with 4.0 M HCl in 1,4-dioxane (275 μL, 1.10 mmol). The dihydrochloride derivative thus obtained was dissolved in H₂O (1 mL), treated with NaHCO₃ (21.0 mg, 0.25 mmol) and MeOH (1 mL), then evaporated to dryness. The title compound I-29 was obtained after flash chromatography (DCM/MeOH, from 100% DCM, to 9:1 v/v DCM/MeOH) as a yellowish solid (42 mg, 0.11 mmol). Yield: quantitative. ¹H-NMR (400 MHz, DMSO-d₆) δ 2.82 (t, J=9.32 Hz, 1H), 3.09-3.13 (m, 1H), 3.22-3.41 (m, 3H), 3.50 (t, J=7.9 Hz, 1H), 7.22-7.28 (m, 1H), 7.35 (s, 4H), 7.58 (d, J=5.7 Hz, 1H), 7.63 (t, J=7.9 Hz, 1H), 7.92 (d, J=8.0 Hz, 2H), 8.44 (d, J=6.0 Hz, 1H), 9.28 (s, 1H), 10.02 (brs, 1H). UHPLC purity: ≥95%. MS-ESI(+) m/z: 318.6 (M+H); MS-ESI(−) m/z: 316.5 (M−H).

### Example 93: (3R,4S)—N-(1-Methylisoquinolin-5-yl)-4-phenylpyrrolidine-3-carboxamide dihydrochloride (Compound I-30)

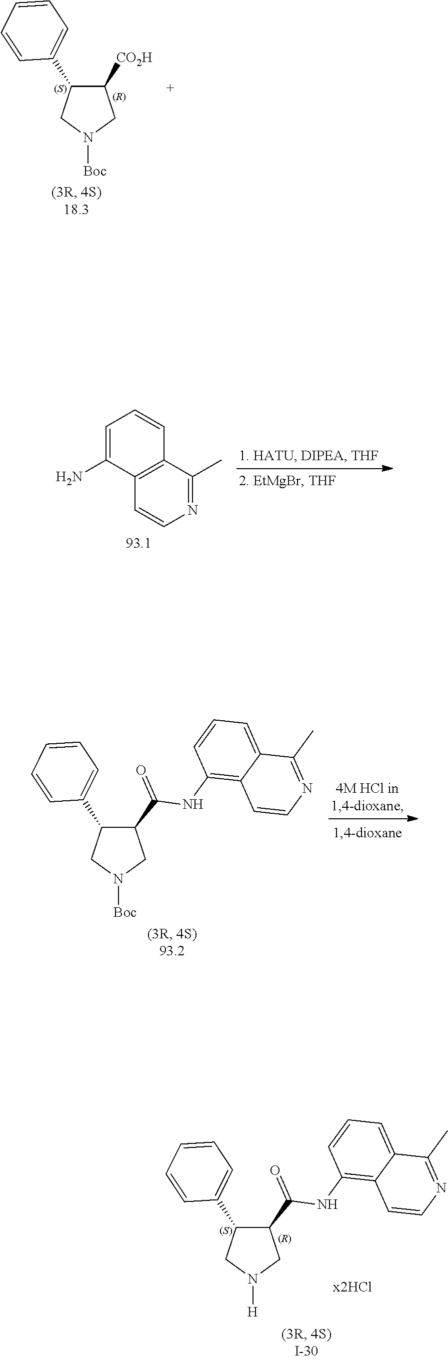

### Step 1: tert-Butyl (±)-trans-4-phenyl-3-(1-methylisoquinolin-5-ylcarbamoyl)pyrrolidine-1-carboxylate (93.2)

Intermediate 93.2 was synthesized according to the procedure described in Step 1 of Example 64 starting from intermediate 18.3 (50 mg, 0.17 mmol), HATU (78 mg, 0.20 mmol), DIPEA (90 μL, 0.51 mmol), intermediate 93.1 (41 mg, 0.26 mmol), and 3.0 M EtMgBr in Et₂O (172 μL, 0.51 mmol) in THF (1 mL+1 mL). The intermediate 93.2 was obtained after flash chromatography (DCM/MeOH, from 100% DCM, to 95:5 v/v DCM/MeOH) (20 mg, 0.05 mmol). Yield: 29%. MS-ESI(+) m/z: 432.7 (M+H); MS-ESI(−) m/z: 430.6 (M−H).

### Step 2: (3R,4S)—N-(1-Methylisoquinolin-5-yl)-4-phenylpyrrolidine-3-carboxamide dihydrochloride, (Compound I-30)

Compound I-30 was synthesized according to the procedure described in Step 2 of Example 64 starting from a solution of intermediate 93.2 (20 mg, 0.05 mmol) in 1,4-dioxane (0.4 mL) which was reacted with 4.0 M HCl in 1,4-dioxane (116 μL, 0.46 mmol). The title compound I-30 (12 mg, 0.03 mmol) was obtained as a yellow solid. Yield: 60%. ¹H-NMR (400 MHz, DMSO-d₆) δ 3.09 (s, 3H), 3.25-3.47 (m, 2H), 3.60-3.67 (m, 3H), 3.68-3.75 (m, 1H), 7.27 (d, J=7.0 Hz, 1H), 7.32 (t, J=7.0 Hz, 2H), 7.66 (d, J=6.7 Hz, 1H), 7.86 (t, J=8.3 Hz, 1H), 8.07 (d, J=7.7 Hz, 1H), 8.29 (d, J=7.1 Hz, 2H), 9.63 (brs, 1H), 9.96 (brs, 1H), 10.67 (s, 1H). UHPLC purity: ≥95%. MS-ESI(+) m/z: 332.6 (M+H); MS-ESI(−) m/z: 330.5 (M−H).

### Example 94: (3R,4S)-4-Phenyl-N-(pyridin-4-ylmethyl)pyrrolidine-3-carboxamide dihydrochloride (Compound I-31)

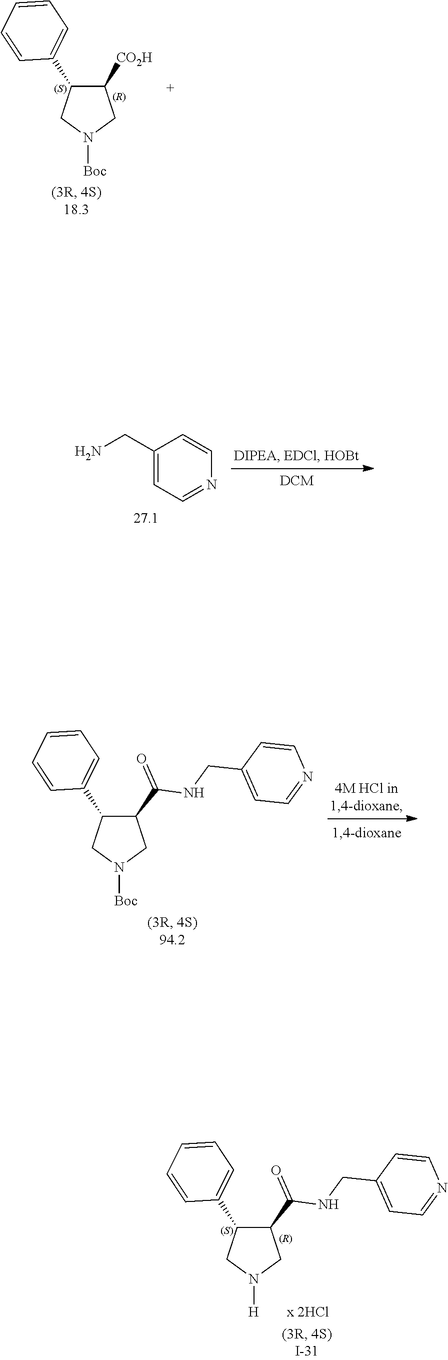

### Step 1: tert-Butyl (3R,4S)-4-phenyl-3-[(pyridin-4-ylmethyl)carbamoyl]pyrrolidine-1-carboxylate (94.2)

DIPEA (0.14 mL, 0.82 mmol), EDCI (157 mg, 0.82 mmol), and HOBt (111 mg, 0.82 mmol) were added to a solution of intermediate 18.3 (160 mg, 0.55 mmol) in DCM (10 mL) and stirring was continued at r.t. for 30 min. Intermediate 94.1 (0.083 mL, 0.82 mmol) was then added and stirring continued for an additional 16 h. The solvent was removed under vacuo. The crude was taken up with H₂O, then extracted with EtOAc (3×20 mL). The collected organic layers were washed with brine (20 mL), dried over Na₂SO₄, and concentrated under reduced pressure. After purification by flash chromatography (DCM/MeOH from 100 DCM to 97:3 v/v DCM/MeOH), the title intermediate 94.2 (120 mg, 0.14 mmol) was obtained as a crystalline powder. Yield 57%.

### Step 2: (3R,4S)-4-Phenyl-N-(pyridin-4-ylmethyl)pyrrolidine-3-carboxamide dihydrochloride (Compound I-31)

Compound I-31 was prepared following the procedure described in Step 2 of Example 64 starting from a solution of intermediate 94.2 (120 mg, 0.31 mmol) in 1,4-dioxane (5 mL) and 4M HCl in 1,4-dioxane (0.7 mL, 3.1 mmol). Stirring was continued at r.t. for 16 h. The title compound I-31 (100 mg, 0.28 mmol) was obtained as a white crystalline powder as dihydrochloride salt. Yield: 91%. ¹H-NMR (400 MHz, DMSOd₆) δ 3.22-3.36 (m, 3H), 3.52-3.59 (m, 1H), 3.64-3.68 (m, 2H), 4.37 (dd, J=17.5 Hz, J=5.58 Hz, 1H), 4.54 (dd, J=17.5 Hz, J=6.1 Hz, 1H), 7.34-7.42 (m, 5H), 7.49 (d, J=6.5 Hz, 2H), 8.71 (d, J=6.6 Hz, 2H), 9.12 (t, J=5.8 Hz, 1H), 9.9 (brs, 1H), 10.05 (brs, 1H). HPLC purity: 98%. MS-ESI(+) m/z: 282.4 (M+H); MS-ESI(−) m/z: 280.5 (M−H).

### Example 95: (3R,4S)-4-Phenyl-N-(thieno[2,3-c]pyridin-3-yl)pyrrolidine-3-carboxamide dihydrochloride Compound I-32)

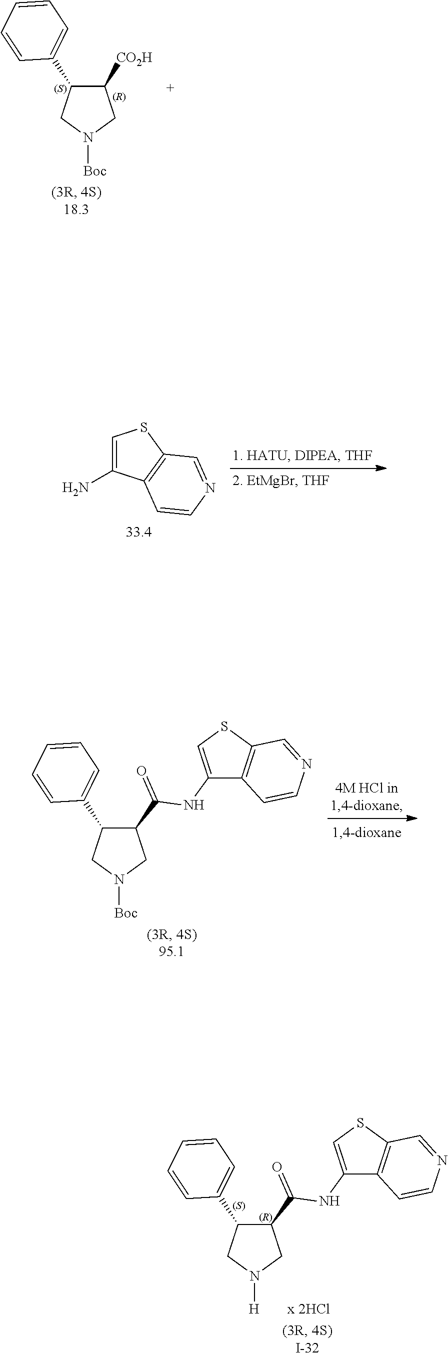

### Step 1: tert-Butyl (3R,4S)-3-phenyl-4-(thieno[2,3-c]pyridin-3-ylcarbamoyl)pyrrolidine-1-carboxylate (70.1)

Intermediate 95.1 was synthesized according to the procedure described in Step 1 of Example 64 starting from intermediate 18.3 (50 mg, 0.17 mmol) which was reacted with HATU (78 mg, 0.21 mmol), DIPEA (90 μL, 0.51 mmol), intermediate 33.4 (39 mg, 0.26 mmol), and 3.0 M EtMgBr in Et₂O (172 μL, 0.51 mmol) in THF (1 mL+1 mL). The intermediate 95.1 (32 mg, 0.17 mmol) was obtained after work-up and flash chromatography (DCM/MeOH, from 100% DCM, to 95:5 v/v DCM/MeOH). Yield: 47%. MS-ESI(+) m/z: 424.7 (M+H); MS-ESI(−) m/z: 422.7 (M−H).

### Step 2: (3R,4S)-4-Phenyl-N-(thieno[2,3-c]pyridin-3-yl)pyrrolidine-3-carboxamide dihydrochloride (Compound I-32)

Compound I-32 was synthesized according to the procedure described in Step 2 of Example 64 starting from a solution of intermediate 95.1 (30 mg, 0.07 mmol) in 1,4-dioxane (0.5 mL) which was reacted with 4.0 M HCl in 1,4-dioxane (177 μL, 0.71 mmol). The title compound I-32 (21 mg, 0.06 mmol) was obtained as a white solid. Yield: 86%. ¹H-NMR (400 MHz, DMSO-d₆) δ 3.27-3.38 (m, 2H), 3.73-3.85 (m, 4H), 7.27-7.30 (m, 1H), 7.33-7.37 (m, 2H), 7.42-7.45 (m, 2H), 8.61 (d, J=6.4H, 1H), 8.71 (d, J=6.3 Hz, 1H), 8.78 (s, 1H), 9.62 (brs, 1H), 9.69 (s, 1H), 9.97 (brs, 1H), 11.25 (s, 1H). UHPLC purity: ≥95%. MS-ESI(+) m/z: 324.5; (M+H) MS-ESI(−) m/z: 322.4 (M−H).

### Example 96: (3R,4S)—N-Benzyl-4-phenylpyrrolidine-3-carboxamide hydrochloride (Compound I-33)

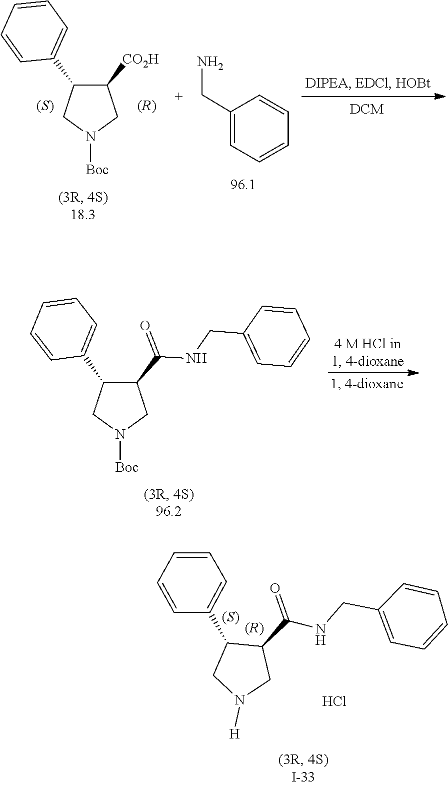

### Step 1: tert-Butyl (3R,4S)-4-phenyl-3-(benzylcarbamoyl)pyrrolidine-1-carboxylate (96.2)

Intermediate 96.2 was prepared according to the procedure described in Step 1 of Example 94 and starting from a solution of intermediate 18.3 (160 mg, 0.51 mmol), DIPEA (0.13 mL, 0.77 mmol), HOBt (111 mg, 0.82 mmol), EDCI (148 mg, 0.77 mmol), and intermediate 96.1 (150 mg, 0.51 mmol) in DCM (15 mL). After purification by flash chromatography (DCM/MeOH from 100% DCM to 98.5/1.5 v/v DCM/MeOH), the intermediate 96.2 (149 mg, 0.39 mmol) was obtained as a colorless foam. Yield 77%.

### Step 2: (3R,4S)—N-Benzyl-4-phenylpyrrolidine-3-carboxamide hydrochloride (Compound I-33)

Compound I-33 was prepared following the procedure described in Step 2 of Example 64 starting from a solution of intermediate 96.2 (134 mg, 0.34 mmol) in 1,4-dioxane (5 mL) and 4M HCl in 1,4-dioxane (0.85 mL, 3.4 mmol). Stirring was continued at r.t. for 16 h. The title compound I-33 (100 mg, 0.28 mmol) was obtained as a white crystalline powder as dihydrochloride salt. Yield: 46%. ¹H-NMR (400 MHz, DMSOd₆) δ 3.21-3.26 (m, 3H), 3.56-3.70 (m, 3H), 4.08 (dd, J=15.4 Hz, J=5.2 Hz, 1H), 4.35 (dd, J=15.4 Hz, J=6.61 Hz, 1H), 6.93-6.95 (m, 2H), 7.16-7.25 (m, 3H), 7.31-7.39 (m, 5H), 8.73 (t, J=5.5 Hz, 1H), 9.62 (brs, 1H), 9.90 (brs, 1H). UHPLC purity: ≥95%. MS-ESI(+) m/z: 281.4 (M+H).

### Example 97: (3R,4S)—N,4-Diphenylpyrrolidine-3-carboxamide (Compound I-34)

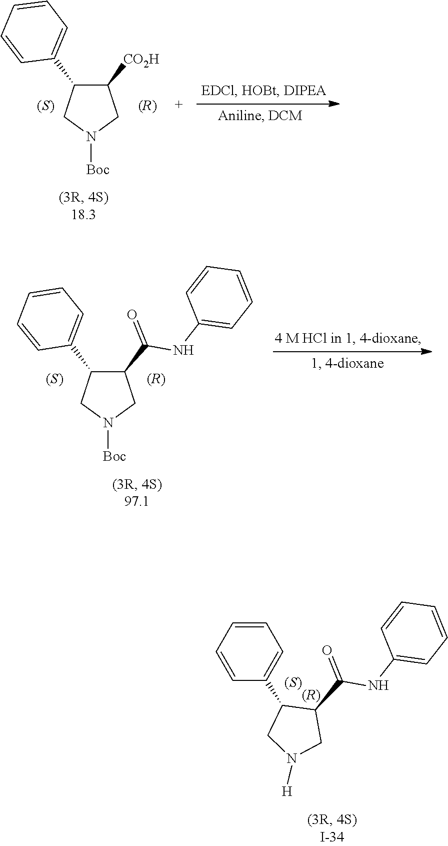

### Step 1: tert-Butyl (3R,4S)-4-phenyl-3-(phenylcarbamoyl)pyrrolidine-1-carboxylate (97.1)

Intermediate 97.1 was prepared according to the procedure described in Step 1 of Example 94 and starting from a solution of intermediate 18.3 (150 mg, 0.51 mmol), DIPEA (0.13 mL, 0.77 mmol), HOBt (104 mg, 0.77 mmol), EDCI (147 mg, 0.77 mmol), and aniline (0.056 mL, 0.62 mmol) in DCM (15 mL). After purification by flash chromatography (DCM/MeOH from 100% DCM to 99:1 v/v DCM/MeOH), the intermediate 97.1 (140 mg, 0.39 mmol) was obtained as a colorless foam. Yield 74%.

### Step 2: (3R,4S)—N,4-Diphenylpyrrolidine-3-carboxamide, (Compound I-34)

Compound I-34 was prepared following the procedure described in Step 2 of Example 64 starting from a solution of intermediate 97.1 (140 mg, 0.38 mmol) in 1,4-dioxane (5 mL) and 4M HCl in 1,4-dioxane (0.95 mL, 3.8 mmol). Stirring was continued at r.t. for 16 h. After purification by reverse phase flash chromatography (H₂O/MeOH from 100% H₂O to H₂O/MeOH 60:40, v/v). The title compound I-34 (50 mg, 0.18 mmol) was obtained as a white powder Yield: 49%. ¹H-NMR (400 MHz, DMSOd₆) δ 2.70-2.78 (m, 1H), 2.95-3.05 (m, 2H), 3.27-3.33 (m, 2H), 3.47-3.51 (m, 1H), 7.00 (t, J=7.4 Hz, 1H), 7.17-7.29 (m, 8H), 7.56 (d, J=8.3 Hz, 2H), 9.90 (m, 1H). HPLC purity: ≥95%. MS-ESI(+) m/z: 267.3 (M+H); MS-ESI(−) m/z: 265.2 (M−H).

### Example 98: (3R,4S)—N-[(1-Methylpiperidin-4-yl)methyl]-4-phenylpyrrolidine-3-carboxamide dihydrochloride (Compound I-35)

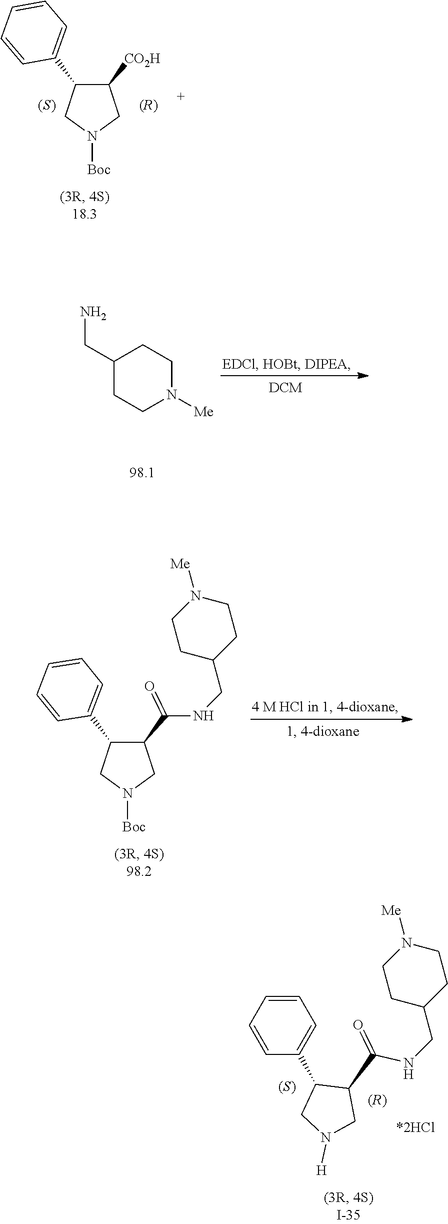

### Step 1: tert-Butyl (3R,4S)-4-phenyl-3-{[(1-methylpiperidin-4-yl)methyl]carbamoyl}pyrrolidine-1-carboxylate (98.2)

Intermediate 98.2 was prepared according to the procedure described in Step 1 of Example 94 and starting from a solution of intermediate 18.3 (156 mg, 0.54 mmol), DIPEA (0.14 mL, 0.81 mmol), HOBt (109 mg, 0.81 mmol), EDCI (155 mg, 0.81 mmol), and intermediate 98.1 (82 mg, 0.63 mmol) in DCM (15 mL). After purification by reverse phase flash chromatography (H₂O/MeOH from 20% MeOH to 100% MeOH), the intermediate 98.2 (160 mg, 0.39 mmol) was obtained as a colorless foam. Yield 78%.

### Step 2: (3R,4S)—N-[(1-Methylpiperidin-4-yl)methyl]-4-phenylpyrrolidine-3-carboxamide dihydrochloride (Compound I-35)

Compound I-35 was prepared following the procedure described in Step 2 of Example 64 starting from a solution of intermediate 98.2 (160 mg, 0.39 mmol) in 1,4-dioxane (5 mL) and 4M HCl in 1,4-dioxane (0.99 mL, 3.98 mmol). Stirring was continued at r.t. for 16 h. The title compound I-35 (90 mg, 0.24 mmol) was obtained as white crystals. Yield: 61%, ¹H-NMR (400 MHz, DMSOd₆) δ 1.22-1.29 (m, 3H), 1.41-1.47 (m, 2H), 2.62-2.74 (m, 6H), 2.98-3.01 (m, 2H), 3.12-3.22 (m, 5H), 3.50-3.63 (m, 2H), 7.26-7.36 (m, 5H), 8.31 (t, J=5.6 Hz, 1H), 9.65 (brs, 1H), 9.97 (brs, 1H), 10.50 (brs, 1H). HPLC purity: >95%. MS-ESI(+) m/z: 302.5 (M+H).

### Example 99: (3R,4S)—N-[(1,4-trans)-4-Hydroxycyclohexyl]-4-phenylpyrrolidine-3-carboxamide hydrochloride (Compound I-36)

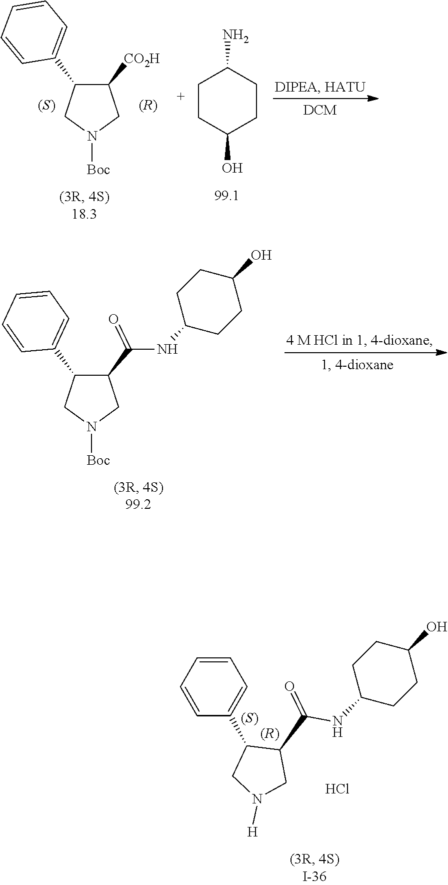

### Step 1: tert-Butyl (3R,4S)-4-phenyl-3-{[(1,4-trans)-4-hydroxycyclohexyl]carbamoyl}pyrrolidine-1-carboxylate (99.2)

DIPEA (0.13 mL, 0.77 mmol) and HATU (293 mg, 0.77 mmol) were added to a solution of intermediate 18.3 (150 mg, 0.51 mmol) in DCM (15 mL), and stirring was continued at r.t. for 1 h. Intermediate 99.1 (89 mg, 0.77 mmol) was then added, and stirring was continued for additional 16 h. The solvent was removed under vacuo. The crude was taken up with H₂O, extracted with EtOAc (3×20 mL). The collected organic layers were washed with brine (20 mL), dried over Na₂SO₄, and concentrated under reduced pressure. After purification by flash chromatography (DCM/MeOH from 100% DCM to 95:5 v/v DCM/MeOH), the intermediate 99.2 (170 mg, 0.43 mmol) was obtained as white crystals. Yield 86%.

### Step 2: (3R,4S)—N-[(1,4-Trans)-4-hydroxycyclohexyl]-4-phenylpyrrolidine-3-carboxamide hydrochloride (Compound I-39)

Compound I-36 was prepared following the procedure described in Step 2 of Example 64 starting from a solution of intermediate 99.2 (170 mg, 0.44 mmol) in 1,4-dioxane (5 mL) and 4M HCl in 1,4-dioxane (1.1 mL, 4.4 mmol). Stirring was continued at r.t. for 16 h. The title compound I-36 (100 mg, 0.3 mmol) was obtained as a white powder. Yield: 70%. ¹H-NMR (400 MHz, DMSOd₆) δ 0.90-0.97 (m, 1H), 1.08-1.15 (m, 3H), 1.25-1.30 (m, 1H), 1.49 (d, J=12.3 Hz, 1H), 1.66-1.73 (m, 3H), 3.02-3.06 (m, 1H), 3.17-3.29 (m, 3H), 3.51-3.55 (m, 2H), 3.62-3.67 (m, 1H), 4.52 (brs, 1H), 7.25-7.35 (m, 5H), 7.99 (d, J=7.7 Hz, 1H), 9.60 (brs, 2H). HPLC purity: >95%. MS-ESI(+) m/z: 289.4 (M+H).

### Example 100: (3R,4S)—N-(Biphenyl-3-yl)-4-phenylpyrrolidine-3-carboxamide hydrochloride (Compound I-37)

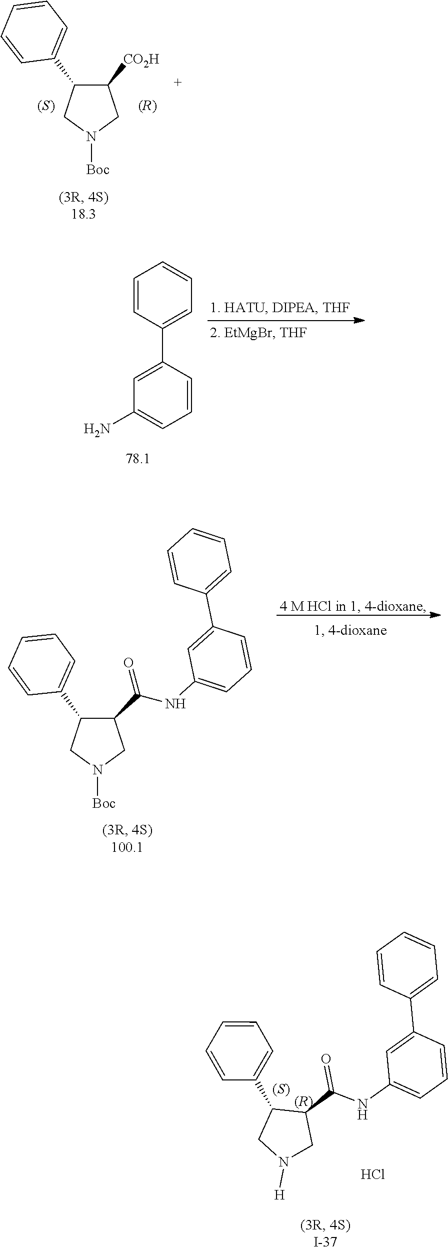

### Step 1: tert-Butyl (3R,4S)-4-phenyl-3-[(biphenyl-3-yl)carbamoyl]pyrrolidine-1-carboxylate (100.1)

Intermediate 100.1 was prepared according to the procedure described in Step 1 of Example 64 starting from a solution of intermediate 18.3 (150 mg, 0.51 mmol), HATU (235 mg, 0.62 mmol), DIPEA (0.27 mL, 1.53 mmol), 3.0 M EtMgBr in Et₂O (0.51 mL, 1.53 mmol), and intermediate 78.1 (112 mg, 0.66 mmol) in THF (5 mL+5 mL). Stirring was continued at r.t. for 16 h. After purification by flash chromatography (PET/EtOAc from 100% PET to 80:20 v/v PET/EtOAc), the intermediate 100.1 (80 mg, 0.18 mmol) was obtained as a colorless oil. Yield 35%.

### Step 2: (3R,4S)—N-(Biphenyl-3-yl)-4-phenylpyrrolidine-3-carboxamide hydrochloride (Compound I-37)

Compound I-37 was prepared following the procedure described in Step 2 of Example 64 starting from a solution of intermediate 100.1 (80 mg, 0.18 mmol) in 1,4-dioxane (5 mL) and 4M HCl in 1,4-dioxane (0.45 mL, 1.81 mmol). Stirring was continued at r.t. for 16 h. The title compound I-37 (30 mg, 0.079 mmol) was obtained as a white powder. Yield: 44%. ¹H-NMR (400 MHz, DMSOd₆) δ 3.26-3.34 (m, 2H), 3.45-3.47 (m, 1H), 3.71-3.75 (m, 3H), 7.25-7.27 (m, 1H), 7.33-7.36 (m, 5H), 7.38-7.39 (m, 2H), 7.42-7.45 (m, 2H) 7.53-7.55 (m, 3H), 7.87 (s, 1H), 9.65 (brs, 1H), 9.95 (brs, 1H), 10.52 (s, 1H). HPLC purity: >95%. MS-ESI(+) m/z: 343.5 (M+H); MS-ESI(−) m/z: 341.3 (M−H).

### Example 101: (3R,4S)—N-(Isoquinolin-3-yl)-4-phenylpyrrolidine-3-carboxamide dihydrochloride (Compound I-38)

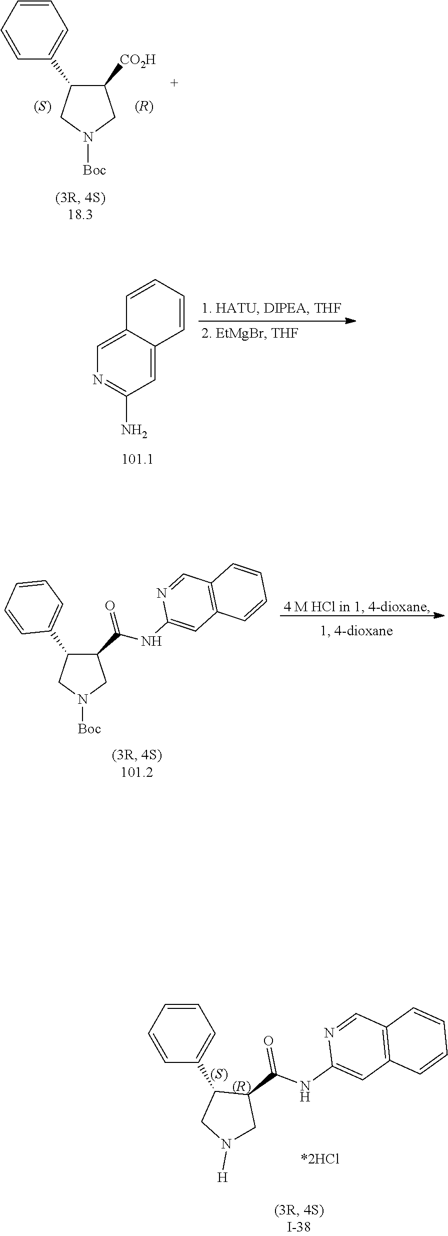

### Step 1: tert-Butyl (3R,4S)-4-phenyl-3-(isoquinolin-3-ylcarbamoyl)pyrrolidine-1-carboxylate (101.2)

Intermediate 101.2 was synthesized according to the procedure described in Step 1 of Example 64 starting from intermediate 18.3 (100 mg, 0.34 mmol), HATU (157 mg, 0.41 mmol), DIPEA (179 μL, 1.03 mmol), intermediate 101.1 (74 mg, 0.51 mmol), and 3.0 M EtMgBr in Et₂O (343 μL, 1.03 mmol) in THF (2 mL+2 mL). The intermediate 101.2 (91 mg, 0.22 mmol) was obtained after work-up and flash chromatography (DCM/MeOH, from 100% DCM, to 95:5 v/v DCM/MeOH). Yield: 65%. MS-ESI(+) m/z: 418.3 (M+H); MS-ESI(−) m/z: 416.3 (M−H).

### Step 2: (3R,4S)—N-(Isoquinolin-3-yl)-4-phenylpyrrolidine-3-carboxamide dihydrochloride, (Compound I-38)

Compound I-38 was synthesized according to the procedure described in Step 2 of Example 64 starting from a solution of intermediate 101.2 (80 mg, 0.19 mmol) in 1,4-dioxane (1 mL) which was reacted with 4.0 M HCl in 1,4-dioxane (485 μL, 1.92 mmol). The title compound I-38 (74 mg, 0.19 mmol) was obtained as a white solid. Yield: quantitative. ¹H-NMR (400 MHz, DMSO-d₆) δ 3.15-3.22 (m, 1H), 3.26-3.35 (m, 1H), 3.57-3.74 (m, 4H), 7.19-7.35 (m, 5H), 7.48 (t, J=7.0 Hz, 1H), 7.65 (t, J=8.2 Hz, 1H), 7.83 (d, J=8.3 Hz, 1H), 7.98 (d, J=8.2 Hz, 1H), 8.40 (s, 1H), 9.06 (s, 1H), 9.71 (brs, 1H), 9.88 (brs, 1H), 10.94 (s, 1H). UHPLC purity: ≥95%. MS-ESI(+) m/z: 318.6 (M+H).

### Example 102: (3R,4S)-4-Phenyl-N-[4-(pyridin-3-yl)phenyl]pyrrolidine-3-carboxamide dihydrochloride (Compound I-39)

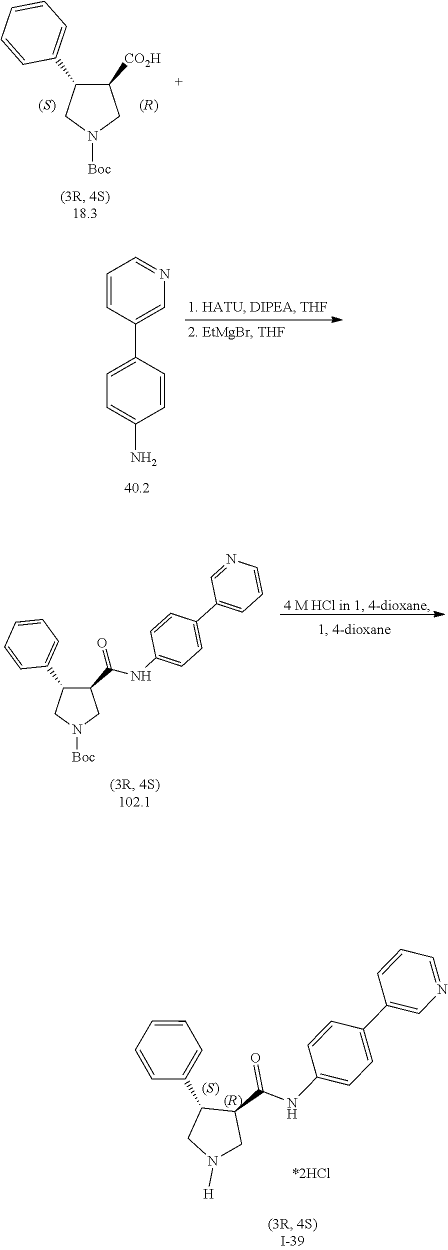

### Step 1: (tert-Butyl (3R,4S)-4-phenyl-3-{[4-(pyridin-3-yl)phenyl]carbamoyl}pyrrolidine-1-carboxylate (102.1)

Intermediate 102.1 was prepared according to the procedure described in Step 1 of Example 64 starting from a solution of intermediate 18.3 (150 mg, 0.51 mmol), HATU (235 mg, 0.62 mmol), DIPEA (0.27 mL, 1.53 mmol), 3.0 M EtMgBr in Et₂O (0.51 mL, 1.53 mmol), and intermediate 40.2 (128 mg, 0.66 mmol) in THF (5+5 mL). Stirring was continued at r.t. 16 h. After purification by flash chromatography (DCM/MeOH from 100% DCM to 97.5:2.5 v/v DCM/MeOH), the title intermediate 102.1 (60 mg, 0.13 mmol) was obtained as white solid. Yield 27%.

### Step 2: (3R,4S)-4-Phenyl-N-[4-(pyridin-3-yl)phenyl]pyrrolidine-3-carboxamide dihydrochloride, (Compound I-39)

Compound I-39 was prepared following the procedure described in Step 2 of Example 64 starting from a solution of intermediate 102.1 (70 mg, 0.16 mmol) in 1,4-dioxane (5 mL) and 4M HCl in 1,4-dioxane (0.4 mL, 1.6 mmol). Stirring was continued at r.t. for 16 h. The title compound I-39 (40 mg, 0.09 mmol) was obtained as a white powder after trituration with Et₂O. Yield: 60%. ¹H-NMR (400 MHz, DMSOd₆) δ 3.49-3.51 (m, 2H), 3.54 (m, 1H), 3.71-3.74 (m, 3H), 7.23-7.27 (m, 1H), 7.33 (t, J=7.3 Hz, 2H), 7.37 (t, J=7.9 Hz, 2H), 7.75 (d, J=7.7 Hz, 2H), 7.80 (d, J=8.7 Hz, 2H), 7.97 (dd, J=8.0 Hz, J=5.7 Hz, 1H), 8.69 (d, J=8.1 Hz, 1H), 8.7 (d, J=5.4 Hz, 1H), 9.1 (s, 1H), 9.75 (brs, 1H), 10.05 (brs, 1H), 10.76 (s, 1H). HPLC purity: >95%. MS-ESI(+) m/z: 344.4 (M+H); MS-ESI(−) m/z: 342.3 (M−H).

### Example 103: (3S,4R)—N-(Isoquinolin-5-yl)-4-phenylpyrrolidine-3-carboxamide dihydrochloride (Compound I-40)

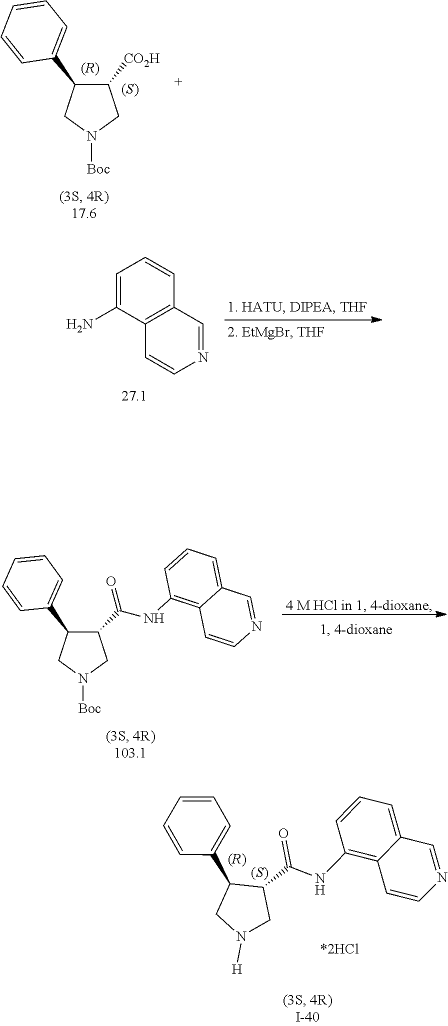

### Step 1: tert-Butyl (3S,4R)-4-phenyl-3-(isoquinolin-5-ylcarbamoyl)pyrrolidine-1-carboxylate (103.1)

Intermediate 103.1 was synthesized according to the procedure described in Step 1 of Example 64 starting from intermediate 17.6 (100 mg, 0.34 mmol), HATU (157 mg, 0.41 mmol), DIPEA (179 μL, 1.03 mmol), intermediate 27.1 (74 mg, 0.51 mmol), and 3.0 M EtMgBr in Et₂O (343 μL, 1.03 mmol) in THF (2 mL+2 mL). The intermediate 103.1 (29 mg, 0.07 mmol) was obtained after work-up and flash chromatography (DCM/MeOH, from 100% DCM, to 95:5 v/v DCM/MeOH). Yield: 21%. MS-ESI(+) m/z: 418.3 (M+H); MS-ESI(−) m/z: 416.3 (M−H);

### Step 2: (3S,4R)—N-(Isoquinolin-5-yl)-4-phenylpyrrolidine-3-carboxamide dihydrochloride (Compound I-40)

Compound I-40 was synthesized according to the procedure described in Step 2 of Example 64 starting from a solution of intermediate 103.1 (29 mg, 0.07 mmol) in 1,4-dioxane (0.7 mL) which was reacted with 4.0 M HCl in 1,4-dioxane (232 μL, 0.70 mmol). The title compound I-40 was obtained as an orange solid (27 mg, 0.07 mmol). Yield: quantitative. ¹H-NMR (400 MHz, DMSO-d₆) δ 3.31 (m, 2H), 3.68-3.95 (m, 4H), 7.29-7.49 (m, 5H), 7.86 (d, J=6.3 Hz, 1H), 7.91 (d, J=7.8 Hz, 1H), 8.14 (d, J=7.6 Hz, 1H), 8.27 (d, J=8.2 Hz, 1H), 8.57 (d, J=6.5 Hz, 1H), 9.65 (brs, 1H), 9.78 (s, 1H), 9.98 (brs, 1H), 10.73 (s, 1H). UHPLC purity: ≥95%. MS-ESI(+) m/z: 318.5 (M+H); MS-ESI(−) m/z: 316.5 (M−H);

### Example 104: (3S,4R)-4-Phenyl-N-[4-(pyridin-3-yl)phenyl]pyrrolidine-3-carboxamide dihydrochloride (Compound I-41)

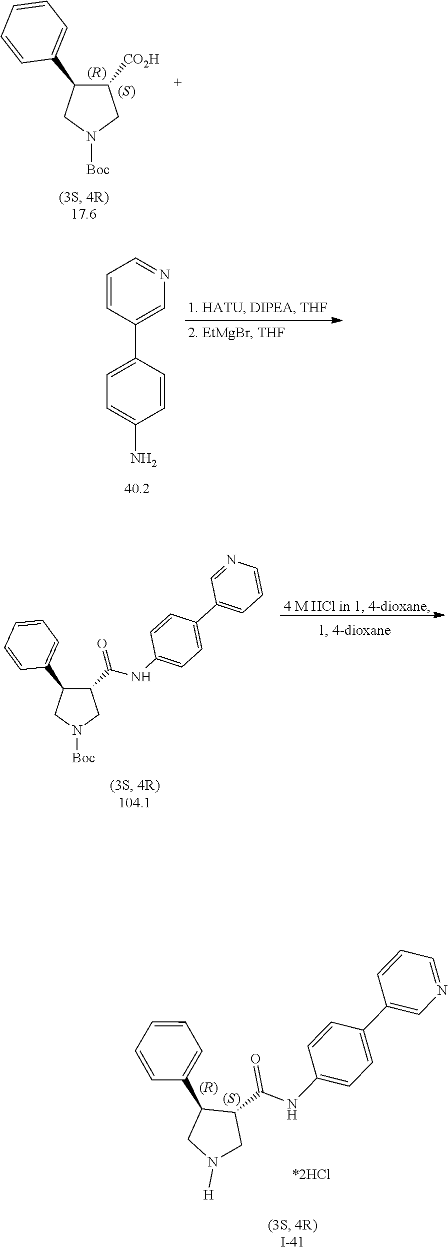

### Step 1: tert-Butyl (3S,4R)-4-phenyl-3-{[4-(pyridin-3-yl)phenyl]carbamoyl}pyrrolidine-1-carboxylate (104.1)

Intermediate 104.1 was prepared according to the procedure described in Step 1 of Example 64 starting from a solution of intermediate 17.6 (150 mg, 0.51 mmol), HATU (252 mg, 0.66 mmol), DIPEA (0.27 mL, 1.53 mmol), 3.0 M EtMgBr in Et₂O (0.51 mL, 1.53 mmol), and intermediate 40.2 (131 mg, 0.78 mmol) in THF (5 mL+5 mL). Stirring was continued at r.t. for 16 h. After purification by flash chromatography (DCM/MeOH from 100% DCM to 97.5:2.5 v/v DCM/MeOH) the intermediate 104.1 (40 mg, 0.13 mmol) was obtained as a white solid. Yield 18%.

### Step 2: (3S,4R)-4-Phenyl-N-[4-(pyridin-3-yl)phenyl]pyrrolidine-3-carboxamide dihydrochloride (Compound I-41)

Compound I-41 was prepared following the procedure described in Step 2 of Example 64 starting from a solution of intermediate 104.1 (40 mg, 0.09 mmol) in 1,4-dioxane (5 mL) and 4M HCl in 1,4-dioxane (0.4 mL, 1.6 mmol). Stirring was continued at r.t. for 16 h. The title Compound I-41 (20 mg, 0.048 mmol) was obtained as a yellowish powder after trituration with Et₂O. Yield: 53%. ¹H-NMR (400 MHz, DMSOd₆) δ 3.26-3.35 (m, 2H), 3.49-3.50 (m, 1H), 3.72-3.76 (m, 3H), 7.25-7.27 (m, 1H), 7.32 (t, J=7.5 Hz, 2H), 7.39-7.40 (m, 2H), 7.75 (d, J=8.3 Hz, 2H), 7.80 (d, J=8.1 Hz, 2H), 7.94 (m, 1H), 8.66 (m, 1H), 8.76 (m, 1H), 9.13 (s, 1H), 9.66 (brs, 1H), 10.03 (brs, 1H), 10.72 (s, 1H). HPLC purity: >95%. MS-ESI(+) m/z: 344.5 (M+H); MS-ESI(−) m/z: 342.3 (M−H).

### Example 105: (3S,4R)—N-(Biphenyl-3-yl)-4-phenylpyrrolidine-3-carboxamide hydrochloride, (Compound I-42)

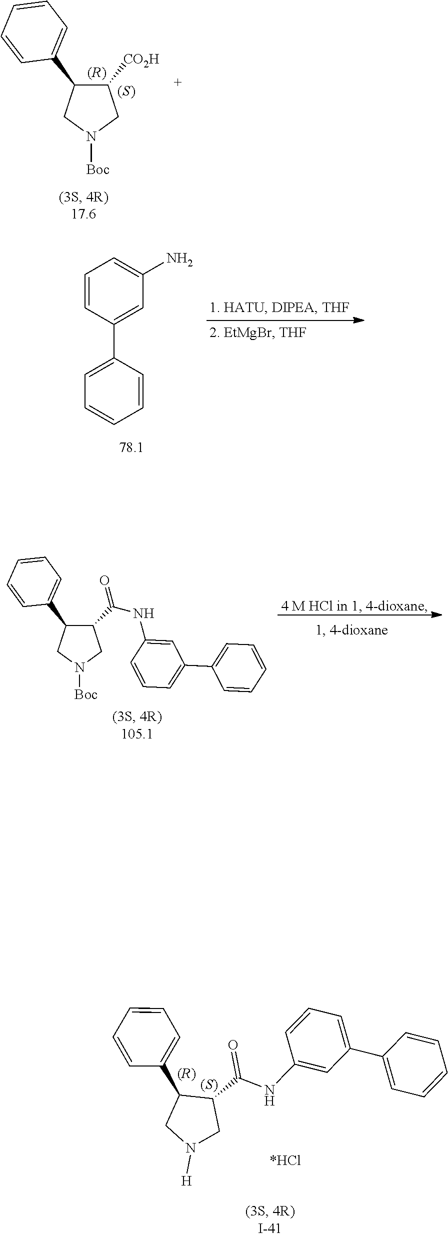

### Step 1: tert-Butyl (3S,4R)-4-phenyl-3-[(biphenyl-3-yl)carbamoyl]pyrrolidine-1-carboxylate (105.1)

Intermediate 105.1 was prepared according to the procedure described in Step 1 of Example 64 starting from a solution of intermediate 17.6 (150 mg, 0.51 mmol), HATU (252 mg, 0.66 mmol), DIPEA (0.27 mL, 1.53 mmol), 3.0 M EtMgBr in Et₂O (0.51 mL, 1.53 mmol) and intermediate 78.1 (103 mg, 0.78 mmol) in THF (5+5 mL). Stirring was continued at r.t. for 16 h. After purification by flash chromatography (PET/EtOAc from 100% PET to 80:20 v/v PET/EtOAc) the intermediate 105.1 (100 mg, 0.13 mmol) was obtained as a colorless oil. Yield 44%.

### Step 2: (3S,4R)—N-(Biphenyl-3-yl)-4-phenylpyrrolidine-3-carboxamide hydrochloride, (Compound I-42)

Compound I-42 was prepared following the procedure described in Step 2 of Example 64 starting from a solution of intermediate 105.1 (120 mg, 0.27 mmol) in 1,4-dioxane (5 mL) and 4M HCl in 1,4-dioxane (0.4 mL, 1.6 mmol). Stirring was continued at r.t. for 16 h. The title compound I-42 (50 mg, 0.13 mmol) was obtained as a grey powder after trituration with Et₂O. Yield: 49%. ¹H-NMR (400 MHz, DMSOd₆) δ 3.26-3.35 (m, 2H), 3.43-3.45 (m, 1H), 3.72 (m, 3H), 7.25-7.27 (m, 1H), 7.32-7.39 (m, 6H), 7.44 (t, J=7.3 Hz, 2H), 7.53 (t, J=7.3 Hz, 3H), 7.86 (s, 1H), 9.66 (brs, 1H), 9.95 (brs, 1H), 10.49 (s, 1H). HPLC purity: >95%. MS-ESI(+) m/z: 343.5 (M+H); MS-ESI(−) m/z: 341.4 (M−H).

### Example 106: (3S,4R)—N-(Isoquinolin-3-yl)-4-phenylpyrrolidine-3-carboxamide dihydrochloride, (Compound I-43)

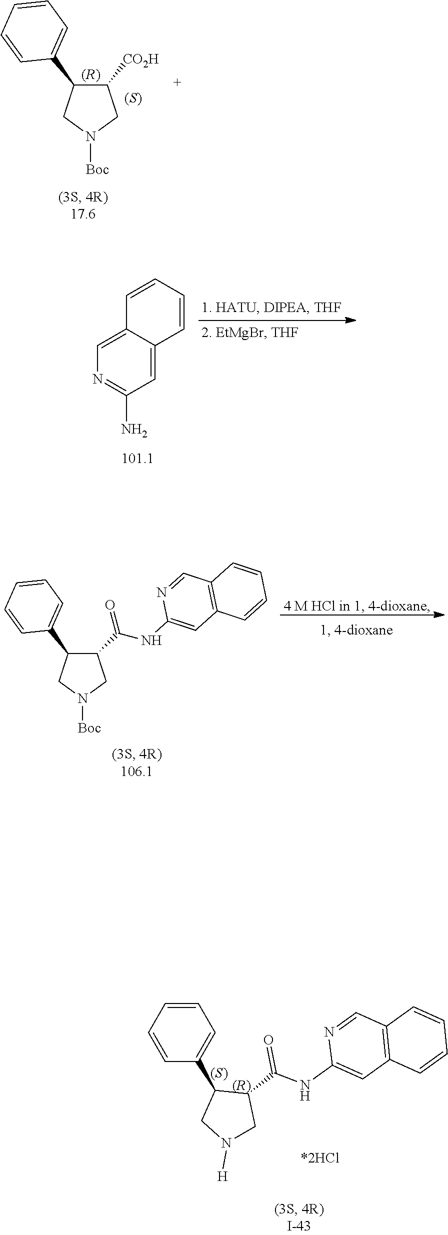

### Step 1: tert-Butyl (3S,4R)-phenyl-3-(isoquinolin-3-ylcarbamoyl)pyrrolidine-1-carboxylate (106.1)

Intermediate 106.1 was synthesized according to the procedure described in Step 1 of Example 64 starting from intermediate 14.6 (100 mg, 0.34 mmol), HATU (157 mg, 0.41 mmol), DIPEA (179 μL, 1.03 mmol), intermediate 101.1 (74 mg, 0.51 mmol), and 3.0 M EtMgBr in Et₂O (343 μL, 1.03 mmol) in THF (2 mL+2 mL). The intermediate 106.1 was obtained after work-up and flash chromatography (DCM/MeOH, from 100% DCM, to 95:5 v/v DCM/MeOH) (115 mg, 0.28 mmol). Yield: 82%. MS-ESI(+) m/z: 418.3 (M+H); MS-ESI(−) m/z: 416.3 (M−H).

### Step 2: (3S,4R)—N-(Isoquinolin-3-yl)-4-phenylpyrrolidine-3-carboxamide dihydrochloride, (Compound I-43)

Compound I-43 was synthesized according to the procedure described in Step 2 of Example 64 starting from a solution of intermediate 106.1 (115 mg, 0.28 mmol) in 1,4-dioxane (3 mL) which was reacted with 4.0 M HCl in 1,4-dioxane (689 μL, 2.8 mmol). The title compound I-43 (109 mg, 0.28 mmol) was obtained as a yellow solid. Yield: quantitative. ¹H-NMR (400 MHz, DMSO-d₆) δ 3.17-3.21 (m, 1H), 3.30-3.34 (m, 1H), 3.59-3-74 (m, 4H), 7.21-7.35 (m, 4H), 7.48 (t, J=8.1 Hz, 1H), 7.65 (t, J=7.6 Hz, 1H), 7.83 (d, J=8.4 Hz, 1H), 7.98 (d, J=8.1 Hz, 1H), 8.40 (s, 1H), 9.05 (s, 1H), 9.61 (brs, 1H), 9.76 (brs, 1H), 10.91 (s, 1H). UHPLC purity: ≥95%. MS-ESI(+) m/z: 318.5 (M+H).

### Example 107: (3R,4R)—N-(Isoquinolin-5-yl)-4-(thiophen-2-yl)pyrrolidine-3-carboxamide dihydrochloride (Compound I-44)

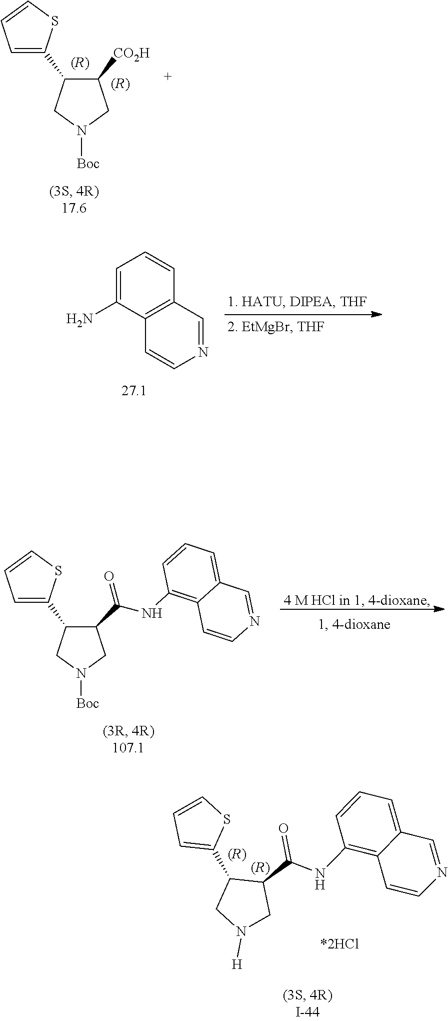

### Step 1: tert-Butyl (3R,4R)-4-(thiophen-2-yl)-3-(isoquinolin-5-ylcarbamoyl)pyrrolidine-1-carboxylate (107.1)

Intermediate 107.1 was prepared according to the procedure described in Step 1 of Example 64 starting from a solution of intermediate 19.5 (250 mg, 0.84 mmol), HATU (416 mg, 1.09 mmol), DIPEA (0.44 mL, 2.52 mmol), 3.0 M EtMgBr in Et₂O (0.84 mL, 2.52 mmol), and intermediate 27.1 (182 mg, 1.26 mmol) in THF (5 mL+5 mL). Stirring was continued at r.t. 16 h. After purification by flash chromatography (DCM/MeOH from 100% DCM to 93:7 v/v DCM/MeOH), the intermediate 107.1 (80 mg, 0.18 mmol) was obtained as a colorless oil. Yield 22%.

### Step 2: (3R,4R)—N-(Isoquinolin-5-yl)-4-(thiophen-2-yl)pyrrolidine-3-carboxamide dihydrochloride, (Compound I-44)

Compound I-44 was prepared following the procedure described in Step 2 of Example 64 starting from a solution of intermediate 107.1 (78 mg, 0.18 mmol) in 1,4-dioxane (5 mL) and 4M HCl in 1,4-dioxane (0.69 mL, 2.76 mmol). Stirring was continued at r.t. for 16 h. The title compound I-44 (50 mg, 0.13 mmol) was obtained as a grey powder after trituration with Et₂O. Yield: 49%. ¹H-NMR (400 MHz, DMSO-d₆) δ 3.33-3.43 (m, 1H), 3.68-3.73 (m, 1H), 3.82-3.84 (m, 2H), 3.94-4.06 (m, 2H), 7.07 (t, J=3.7 Hz, 1H), 7.21 (s, 1H), 7.50 (d, J=5.1 Hz, 1H), 7.96 (t, J=7.4 Hz, 1H), 8.21 (t, J=7.6 Hz, 2H), 8.31 (d, J=7.3 Hz, 1H), 8.65 (d, J=5.3 Hz, 1H), 9.72 (brs, 1H), 9.81 (s, 1H), 10.03 (brs. 1H), 10.93 (s, 1H). UHPLC purity: 93%. MS-ESI(+) m/z: 324.0 (M+H); MS-ESI(−) m/z: 321.9 (M−H).

### Example 108: (±)-trans-N-(Biphenyl-3-yl)-4-(4-methoxyphenyl)pyrrolidine-3-carboxamide hydrochloride (Compound I-45)

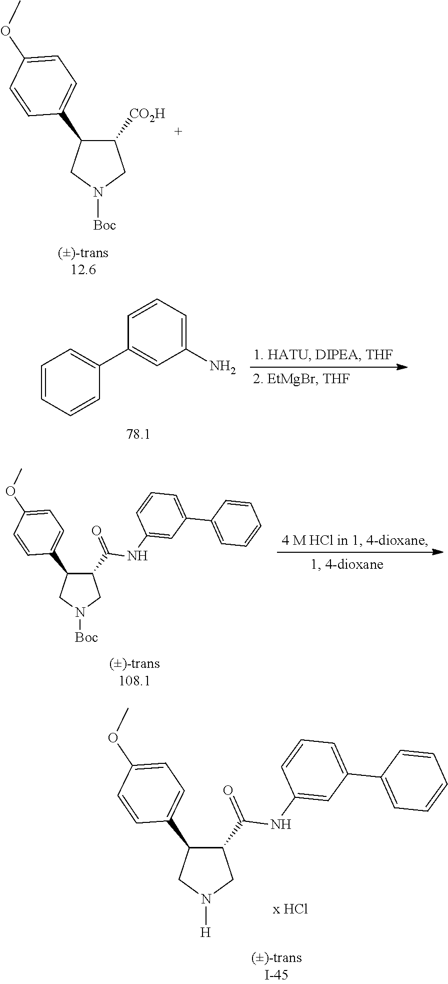 [SMILES: COc1ccc([C@@H]2CN(C(=O)OC(C)(C)C)C[C@H]2C(=O)O)cc1 ; Nc1cccc(-c2ccccc2)c1 ; COc1ccc([C@@H]2CN(C(=O)OC(C)(C)C)C[C@H]2C(=O)Nc2cccc(-c3ccccc3)c2)cc1 ; COc1ccc([C@@H]2CNC[C@H]2C(=O)Nc2cccc(-c3ccccc3)c2)cc1]

### Step 1: tert-Butyl (±)-trans-4-(4-methoxyphenyl)-3-[(biphenyl-3-yl)carbamoyl]pyrrolidine-1-carboxylate (108.1)

Intermediate 108.1 was prepared according to the procedure described in Step 1 of Example 64 starting from a solution of intermediate 12.6 (126 mg, 0.43 mmol), HATU (197 mg, 0.52 mmol), DIPEA (0.22 mL, 1.29 mmol), 3.0 M EtMgBr in Et₂O (0.43 mL, 1.29 mmol), and intermediate 78.1 (88 mg, 0.52 mmol) in THF (5 mL+5 mL). Stirring was continued at r.t. 16 h. After purification by flash chromatography (PET/EtOAc from 100 PET to 80:20 v/v PET/EtOAc), the intermediate 108.1 (30 mg, 0.06 mmol) was obtained as a colorless oil. Yield 15%.

### Step 2: (±)-trans-N-(Biphenyl-3-yl)-4-(4-methoxyphenyl)pyrrolidine-3-carboxamide hydrochloride, (Compound I-45)

Compound I-45 was prepared following the procedure described in Step 2 of Example 64 starting from a solution of intermediate 108.1 (20 mg, 0.04 mmol) in 1,4-dioxane (5 mL) and 4M HCl in 1,4-dioxane (0.1 mL, 0.42 mmol). Stirring was continued at r.t. for 16 h. The title Compound I-45 (15 mg, 0.06 mmol) was the hydrochloride salt as a yellowish powder, after trituration with Et₂O. Yield: 92%. ¹H-NMR (400 MHz, DMSOd₆) δ 3.24 (m, 1H), 3.37-3.41 (m, 2H), 3.69-3.72 (m, 6H), 6.91 (d, J=8.1 Hz, 2H), 7.32 (d, J=8.0 Hz, 2H), 7.35-7.39 (m, 3H), 7.47 (t, J=8.0 Hz, 2H), 7.56 (t, J=8.1 Hz, 3H), 7.88 (s, 1H), 9.44 (brs, 1H), 9.80 (brs, 1H), 10.43 (s, 1H). UHPLC purity: 96%. MS-ESI(+) m/z: 372.8 (M+H); MS-ESI(−) m/z: 370.8 (M−H).

### Example 109: (±)-trans-1-Methyl-4-phenyl-N-[3-(pyridin-3-yl)phenyl]pyrrolidine-3-carboxamide, (Compound I-46)

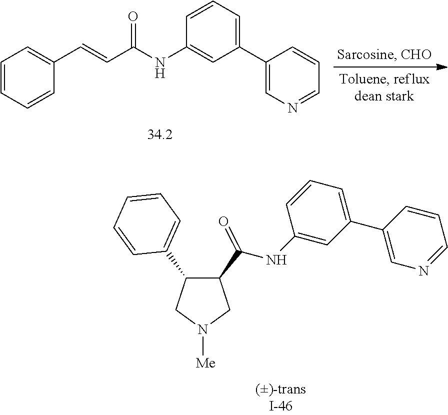 [SMILES: O=C(/C=C/c1ccccc1)Nc1cccc(-c2cccnc2)c1 ; CN1C[C@H](C(=O)Nc2cccc(-c3cccnc3)c2)[C@@H](c2ccccc2)C1]

p-Formaldehyde (181 mg, 5.96 mmol) and sarcosine (334 mg, 3.74 mmol) were added to a solution of intermediate 34.2 (450 mg, 1.49 mmol) in toluene (10 mL) and stirring was continued at reflux with a Dean Stark apparatus for 16 h. The solvent was removed under vacuo and the crude taken up with DCM/MeOH then purified by flash chromatography (DCM/MeOH from 100% DCM to 92:8 v/v DCM/MeOH). The title compound I-46 (220 mg, 0.61 mmol) was obtained as a white powder. Yield 41%. ¹H-NMR (400 MHz, DMSOd₆) δ 2.33 (s, 3H), 2.65 (m, 1H), 2.73 (m, 1H), 2.89 (m, 1H), 3.10-3.15 (m, 2H), 3.73 (m, 1H), 7.21 (m, 1H), 7.31 (m, 4H), 7.40-7.42 (m, 2H), 7.49 (m, 1H), 7.60 (m, 1H), 7.95 (s, 1H), 7.99-8.00 (m, 1H), 8.58 (m, 1H), 8.82 (s, 1H), 10.1 (s, 1H). HPLC purity: 99%. MS-ESI(+) m/z: 357.8 (M+H); MS-ESI(−) m/z: 355.9 (M−H).

### Example 110: (±)-trans-N-Methyl-4-phenyl-N-[3-(pyridin-3-yl)phenyl]pyrrolidine-3-carboxamide (Compound I-47)

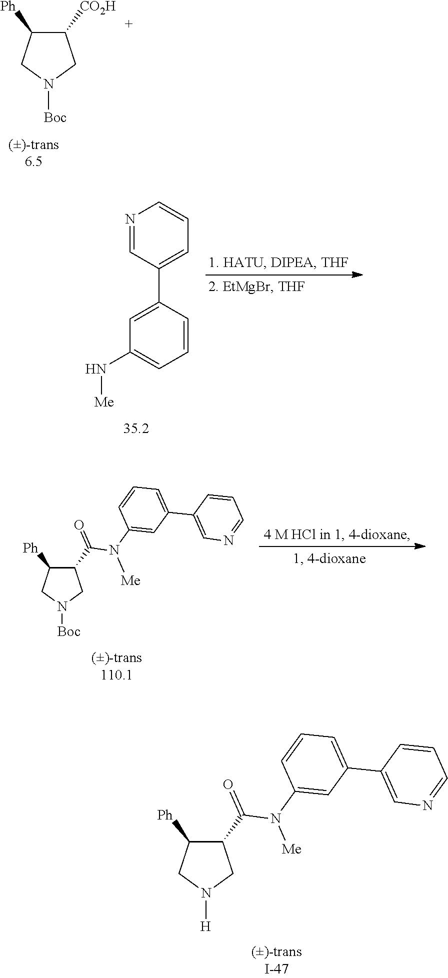 [SMILES: CC(C)(C)OC(=O)N1C[C@@H](C(=O)O)[C@H](c2ccccc2)C1 ; CNc1cccc(-c2cccnc2)c1 ; CN(C(=O)[C@@H]1CN(C(=O)OC(C)(C)C)C[C@H]1c1ccccc1)c1cccc(-c2cccnc2)c1 ; CN(C(=O)[C@@H]1CNC[C@H]1c1ccccc1)c1cccc(-c2cccnc2)c1]

### Step 1: tert-Butyl (±)-trans-4-phenyl-3-{methyl[3-(pyridin-3-yl)phenyl]carbamoyl}pyrrolidine-1-carboxylate (110.1)

Intermediate 110.1 was synthesized according to the procedure described in Step 1 of Example 64 starting from intermediate 6.5 (130 mg, 0.45 mmol), HATU (204 mg, 0.54 mmol), DIPEA (233 μL, 1.34 mmol), intermediate 35.2 (123 mg, 0.67 mmol), and 3.0 M EtMgBr in Et₂O (446 μL, 1.34 mmol) in THF (3 mL+3 mL). The intermediate 110.1 (137 mg, 0.30 mmol) was obtained after work-up and flash chromatography (DCM/MeOH, from 100% DCM, to 95:5 v/v DCM/MeOH). Yield: 67%. MS-ESI(+) m/z: 458.1 (M+H).

### Step 2: (±)-trans-N-Methyl-4-phenyl-N-[3-(pyridin-3-yl)phenyl]pyrrolidine-3-carboxamide, (Compound I-47)

Compound I-47 was synthesized according to the procedure described in Step 2 of Example 64 starting from a solution of intermediate 110.1 (137 mg, 0.30 mmol) in 1,4-dioxane (3 mL) which was reacted with 4.0 M HCl in 1,4-dioxane (749 μL, 3.0 mmol). The title compound I-47 (75 mg, 0.27 mmol) was obtained as a yellow solid. Yield: 70%. ¹H-NMR (400 MHz, CDCl₃) δ 2.97-3.11 (m, 2H), 3.24 (s, 3H), 3.30-3.35 (m, 1H), 3.45-3.49 (m, 1H), 3.58-3.67 (m, 2H), 6.70-6.81 (m, 2H), 7.01-7.10 (m, 5H), 7.33-7.39 (m, 2H), 7.46-7.48 (m, 2H), 7.66 (s, 1H), 8.61-8.65 (m, 2H). UHPLC purity: ≥95%. MS-ESI(+) m/z: 357.8 (M+H).

### Example 111: (±)-trans-(4-Phenylpyrrolidin-3-yl)[3-(pyridin-3-yl)azetidin-1-yl]methanone dihydrochloride, (Compound I-48)

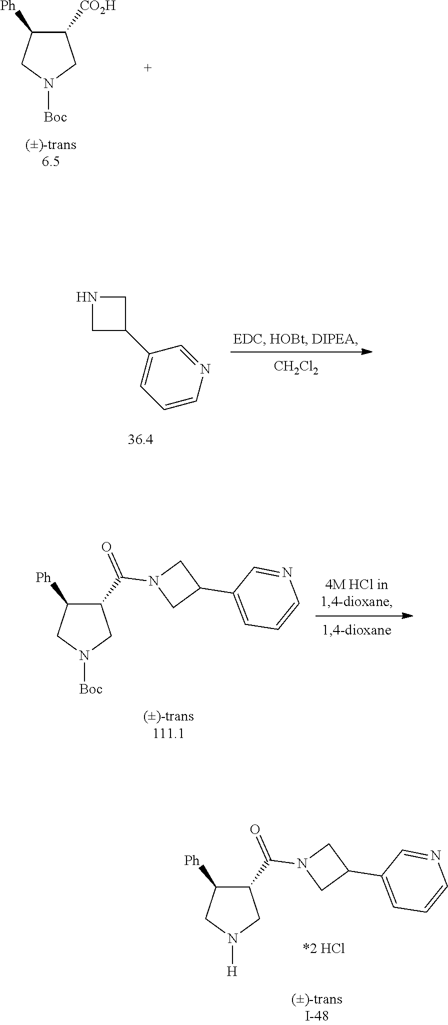 [SMILES: CC(C)(C)OC(=O)N1C[C@@H](C(=O)O)[C@H](c2ccccc2)C1 ; c1cncc(C2CNC2)c1 ; "CH2Cl2" ; CC(C)(C)OC(=O)N1C[C@@H](C(=O)N2CC(c3cccnc3)C2)[C@H](c2ccccc2)C1 ; "1,4-dioxane" ; O=C([C@@H]1CNC[C@H]1c1ccccc1)N1CC(c2cccnc2)C1]

### Step 1: tert-Butyl (±)-trans-phenyl-3-{[3-(pyridin-3-yl)azetidin-1-yl]carbonyl}pyrrolidine-1-carboxylate (111.1)

Intermediate 111.1 was synthesized according to the procedure described in Step 1 of Example 94 from intermediate 6.5 (100 mg, 0.34 mmol), intermediate 36.4 (107 mg, 0.52 mmoL), EDC (99 mg, 0.51 mmol), HOBt (70 mg, 0.51 mmol), and DIPEA (269 μL, 1.54 mmol) in DCM (4 mL+2 mL). The intermediate 111.1 (133 mg, 0.33 mmol) was obtained after work-up and chromatographic purification (DCM/MeOH, from 100% DCM to 95:5 v/v DCM/MeOH). Yield: 95%. MS-ESI(−) m/z: 406.0 (M−H).

### Step 2: (±)-trans-(4-Phenylpyrrolidin-3-yl)[3-(pyridin-3-yl)azetidin-1-yl]methanone dihydrochloride, (Compound I-48)

Compound I-48 was synthesized according to the procedure described in Step 2 of Example 64 from a solution of intermediate 111.1 (133 mg, 0.33 mmol) in 1,4-dioxane (2 mL) which was reacted with 4.0M HCl (816 μL, 3.26 mmol). The title compound I-48 (122 mg, 0.32 mmol) was obtained as a white solid. Yield: 97%. ¹H-NMR (400 MHz, DMSO-d₆) δ 2.91-3.05 (m, 3H), 3.26-3.50 (m, 6H), 3.73-3.83 (m, 2H), 7.12-7.25 (m, 5H), 7.81 (brs, 1H), 8.17-8.23 (m, 1H), 8.41 (s, 1H), 8.71-8.74 (m, 2H), 9.57 (brs, 1H), 9.91 (brs, 1H). UHPLC purity: ≥95%. MS-ESI(+) m/z: 307.1 (M+H).

### Example 112: (±)-trans-N-[3-(Pyridin-3-yl)phenyl]-4-(thiophen-2-yl)pyrrolidine-3-carboxamide dihydrochloride (Compound I-49)

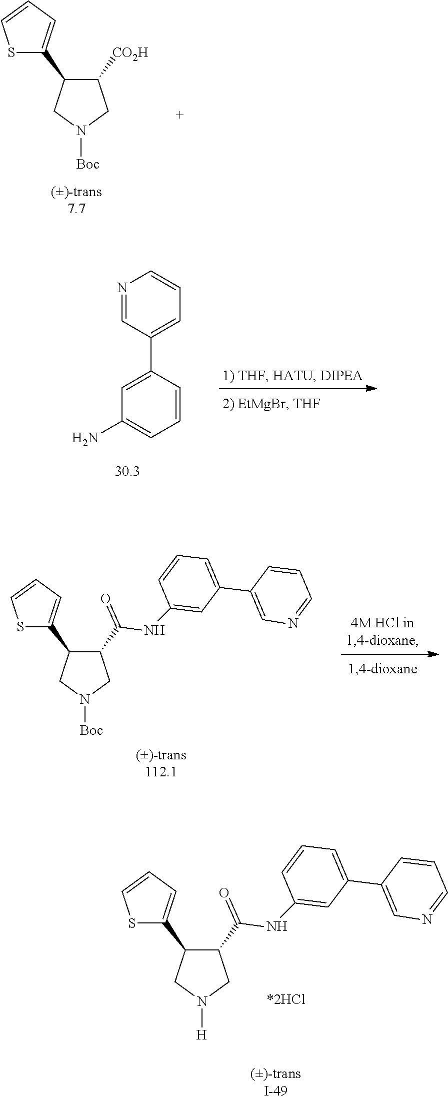 [SMILES: CC(C)(C)OC(=O)N1C[C@@H](C(=O)O)[C@H](c2cccs2)C1 ; Nc1cccc(-c2cccnc2)c1 ; CC(C)(C)OC(=O)N1C[C@@H](C(=O)Nc2cccc(-c3cccnc3)c2)[C@H](c2cccs2)C1 ; "1,4-dioxane" ; O=C(Nc1cccc(-c2cccnc2)c1)[C@@H]1CNC[C@H]1c1cccs1]

### Step 1: tert-Butyl (±)-trans-4-(thiophen-2-yl)-3-{[3-(pyridin-3-yl)phenyl]carbamoyl}pyrrolidine-1-carboxylate (112.1)

Intermediate 112.1 was synthesized according to the procedure described in Step 1 of Example 64 from intermediate 7.7 (150 mg, 0.50 mmol), HATU (230 mg, 0.61 mmol), DIPEA (264 μL, 1.51 mmol), intermediate 30.3 (129 mg, 0.76 mmol), and 3.0M EtMgBr in Et₂O (504 μL, 1.51 mmol) in THF (3 mL+3 mL). The intermediate 112.1 (55 mg, 0.12 mmol) was obtained after work-up and chromatographic purification (DCM/MeOH). Yield: 24%. MS-ESI(−) m/z: 448.1 (M−H).

### Step 2: (±)-trans-N-[3-(Pyridin-3-yl)phenyl]-4-(thiophen-2-yl)pyrrolidine-3-carboxamide dihydrochloride (Compound I-49)

Compound I-49 was synthesized according to the procedure described in Step 2 of Example 64 from a solution of intermediate 112.1 (55 mg, 0.12 mmol) in 1,4-dioxane (1.2 mL) which was reacted with 4.0M HCl in 1,4-dioxane (306 μL, 1.22 mmol). The title compound I-49 (50 mg, 0.12 mmol) was obtained as a yellow solid. Yield: quantitative. ¹H-NMR (400 MHz, DMSO-d₆) δ 3.23-3.39 (m, 2H), 3.46 (q, J=9.6 Hz, 1H), 3.72-3.83 (m, 2H), 4.01 (q, J=10.0 Hz, 1H), 7.01 (m, 1H), 7.14 (d, J=3.2 Hz, 1H), 7.45 (d, J=5.0 Hz, 1H), 7.49-7.56 (m, 2H), 7.72 (d, J=7.6 Hz, 1H), 8.00-8.03 (m, 1H), 8.12 (s, 1H), 8.63 (d, J=7.7 Hz, 1H), 8.84 (d, J=5.1 Hz, 1H), 9.09 (s, 1H), 9.81 (brs, 1H), 10.11 (brs, 1H), 10.90 (s, 1H). UHPLC purity ≥95%. MS-ESI(+) m/z: 349.8 (M+H); MS-ESI(−) m/z: 347.8 (M−H);

### Example 113: (±)-trans-N-[3-(Pyridin-3-yl)phenyl]-4-(tetrahydro-2H-pyran-4-yl)pyrrolidine-3-carboxamide dihydrochloride (Compound I-50)

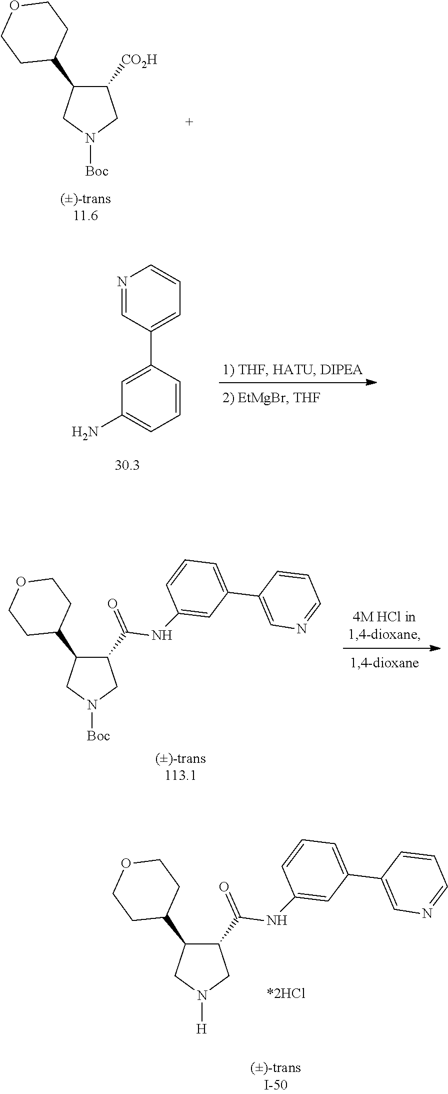 [SMILES: CC(C)(C)OC(=O)N1C[C@@H](C(=O)O)[C@H](C2CCOCC2)C1 ; Nc1cccc(-c2cccnc2)c1 ; CC(C)(C)OC(=O)N1C[C@@H](C(=O)Nc2cccc(-c3cccnc3)c2)[C@H](C2CCOCC2)C1 ; "1,4-dioxane" ; O=C(Nc1cccc(-c2cccnc2)c1)[C@@H]1CNC[C@H]1C1CCOCC1]

### Step 1: tert-Butyl (±)-trans-4-(tetrahydro-2H-pyran-4-yl)-3-{[3-(pyridin-3-yl)phenyl]carbamoyl}pyrrolidine-1-carboxylate (113.1)

Intermediate 113.1 was prepared according to the procedure described in Step 1 of Example 64 starting from a solution of intermediate 11.6 (250 mg, 0.83 mmol), HATU (412 mg, 1.1 mmol), DIPEA (0.43 mL, 2.49 mmol), 3.0 M EtMgBr in Et₂O (0.83 mL, 2.49 mmol), and 30.3 (211 mg, 1.24 mmol) in THF (5 mL+5 mL). Stirring was continued at r.t. for 16 h. After flash purification by flash chromatography (DCM/MeOH from 100% DCM to 92:8 v/v of DCM/MeOH). The intermediate 113.1 (100 mg, 0.18 mmol) was obtained as a colorless oil. Yield 22%.

### Step 2: (±)-trans-N-[3-(Pyridin-3-yl)phenyl]-4-(tetrahydro-2H-pyran-4-yl)pyrrolidine-3-carboxamide dihydrochloride (Compound I-50)

Compound I-50 was prepared following the procedure described in Step 2 of Example 64 starting from a solution of intermediate 113.1 (100 mg, 0.22 mmol) in 1,4-dioxane (5 mL) and 4M HCl in 1,4-dioxane (0.83 mL, 3.32 mmol). Stirring was continued at r.t. for 16 h. The title compound I-50 (30 mg, 0.07 mmol) was obtained as the dihydrochloride salt as a yellowish powder after trituration with Et₂O. Yield: 32%. ¹H-NMR (400 MHz, DMSOd₆) δ 1.19-1.29 (m, 3H), 1.57-1.65 (m, 4H), 2.98-3.03 (m, 1H), 3.15-3.28 (m, 4H), 3.79-3.84 (m, 4H), 7.5-7.55 (m, 2H), 7.75 (dt, J=7.4 Hz, J=1.9 Hz, 1H), 7.97 (dd, J=8.15 Hz, J=5.4 Hz, 1H), 8.11 (s, 1H), 8.57 (d, J=7.9 Hz, 1H), 8.82 (dd, J=5.4 Hz, J=1.08 Hz, 1H), 9.07 (d, J=1.9 Hz, 1H), 9.35 (brs, 1H), 9.70 (brs, 1H), 10.87 (s, 1H). HPLC purity: 85%. MS-ESI(+) m/z: 352.0 (M+H); MS-ESI(−) m/z: 350.0 (M−H).

### Example 114: 4-Phenyl-N-[3-(pyridin-3-yl)phenyl]-1H-pyrrole-3-carboxamide (Compound I-51)

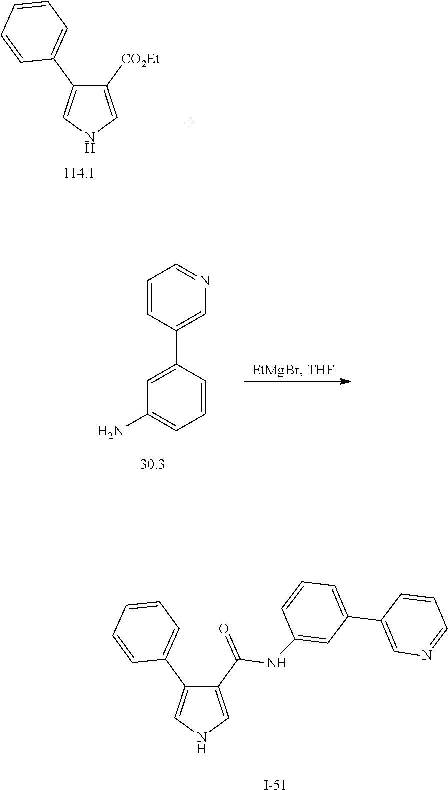

To a stirred solution of intermediate 114.1 (47 mg, 0.28 mmol) in dry THF (1 mL), 3.0 M EtMgBr in Et₂O (186 μL, 0.56 mmol) was added quickly dropwise thereby immediately obtaining a dense suspension which was vigorously stirred at 40° C. for 15 min. A solution of intermediate 30.3 (40 mg, 0.19 mmol) in dry THF (1 mL) was then added, and the mixture thus obtained was reacted at 40° C. for 2 days. The mixture was diluted with EtOAc (10 mL) and washed with H₂O (3×20 mL), 0.5 M aq. citric acid (2×20 mL), and brine (20 mL), dried over Na₂SO₄ and concentrated under reduced pressure. The residue was purified by flash chromatography (DCM/MeOH, from 100% DCM, to 95:5 v/v DCM/MeOH) to provide the title compound I-51 (16 mg, 0.05 mmol). Yield: 26%. ¹H-NMR (400 MHz, DMSO-d₆) δ 7.01-7.03 (m, 1H), 7.17-7.19 (m, 1H), 7.27 (t, J=7.82 Hz, 2H), 7.35-7-51 (m, 6H), 7.72 (d, J=8.2 Hz, 1H), 7.98-8.01 (m, 2H), 8.57 (dd, J=1.4 Hz, J=4.7 Hz, 1H), 8.82-8.83 (m, 1H), 9.78 (s, 1H), 11.41 (s, 1H). UHPLC purity: ≥95%. MS-ESI(+) m/z: 339.8 (M+H); MS-ESI(−) m/z: 337.8 (M−H).

### Example 115: (±)-trans-N-(3-Phenoxyphenyl)-4-phenylpyrrolidine-3-carboxamide dihydrochloride, (Compound I-52)

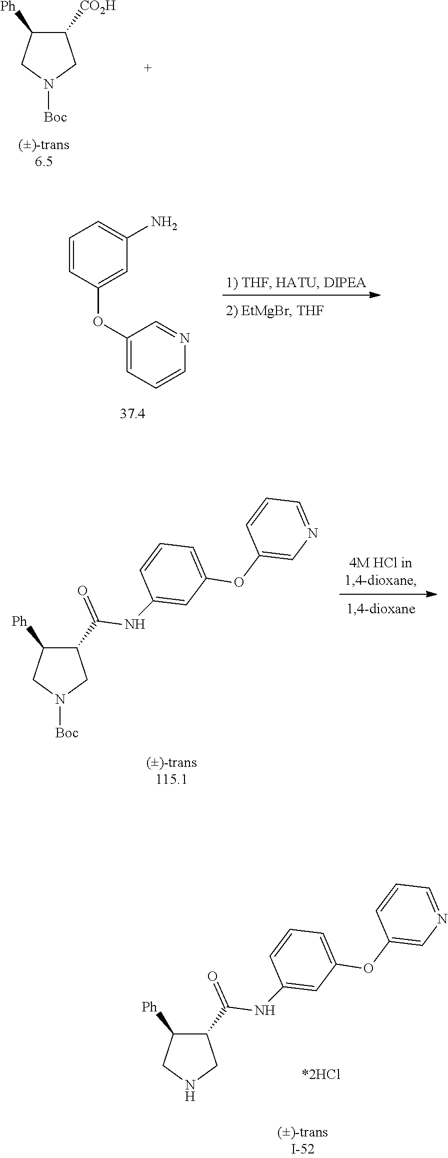 [SMILES: CC(C)(C)OC(=O)N1C[C@@H](C(=O)O)[C@H](c2ccccc2)C1 ; Nc1cccc(Oc2cccnc2)c1 ; CC(C)(C)OC(=O)N1C[C@@H](C(=O)Nc2cccc(Oc3cccnc3)c2)[C@H](c2ccccc2)C1 ; "1,4-dioxane" ; O=C(Nc1cccc(Oc2cccnc2)c1)[C@@H]1CNC[C@H]1c1ccccc1]

### Step 1: tert-Butyl (±)-trans-4-Phenyl-3-[(3-phenoxyphenyl)carbamoyl]pyrrolidine-1-carboxylate (115.1)

Intermediate 115.1 was synthesized according to the procedure described in Step 1 of Example 64 from intermediate 6.5 (104 mg, 0.36 mmol), HATU (163 mg, 0.43 mmol), DIPEA (187 μL, 1.07 mmol), intermediate 37.4 (100 mg, 0.54 mmol), and 3.0M EtMgBr in Et₂O (358 μL, 1.07 mmol) in THF (2 mL+2 mL). The intermediate 115.1 (37 mg, 0.08 mmol) was obtained after work-up and chromatographic purification (DCM/MeOH from 100% DCM to 95:5 v/v DCM/MeOH). Yield: 22%.

### Step 2: (±)-trans-N-(3-Phenoxyphenyl)-4-phenylpyrrolidine-3-carboxamide dihydrochloride, (Compound I-52)

Compound I-52 was synthesized according to the procedure described in Step 2 of Example 64 from a solution of intermediate 115.1 (37 mg, 0.08 mmol) in 1,4-dioxane (0.8 mL) which was reacted with 4.0M HCl in 1,4-dioxane (201 μL, 0.81 mmol). The title compound I-52 (35 mg, 0.08 mmol) was obtained as a pink solid. Yield: quantitative. ¹H-NMR (400 MHz, DMSO-d₆) δ 3.23-3.33 (m, 2H), 3.40-3.46 (m, 1H), 3.66-3.73 (m, 3H), 5.55 (brs, 1H), 6.81-6.83 (m, 1H), 7.26-7.38 (m, 7H), 7.44 (s, 1H), 7.66-7.75 (m, 2H), 8.50 (d, J=4.8 Hz, 1H), 8.54 (d, J=2.2 Hz, 1H), 9.52 (brs, 1H), 9.92 (brs, 1H), 10.59 (s, 1H). UHPLC purity: ≥95%. MS-ESI(+) m/z: 360.0 (M+H); MS-ESI(−) m/z: 358.0 (M−H).

### Example 116: (±)-trans-4-Phenyl-N-[3-(pyrimidin-5-yl)phenyl]pyrrolidine-3-carboxamide trihydrochloride (Compound I-53)

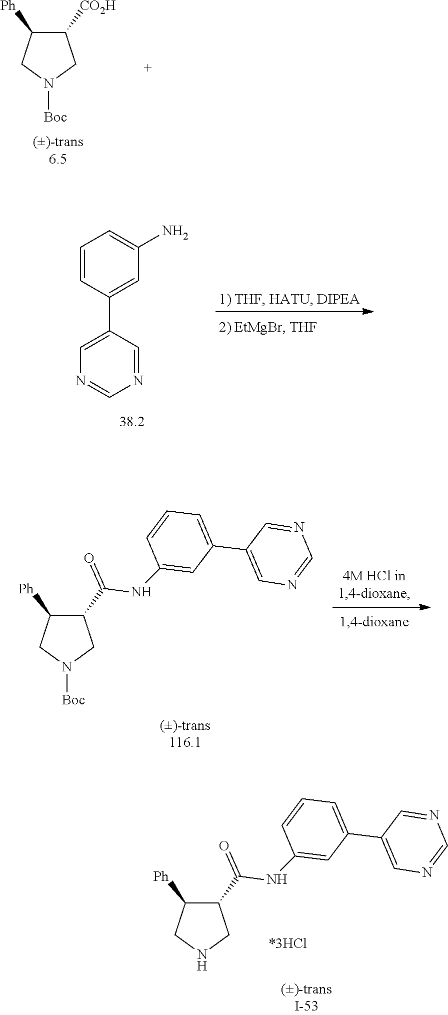 [SMILES: CC(C)(C)OC(=O)N1C[C@@H](C(=O)O)[C@H](c2ccccc2)C1 ; Nc1cccc(-c2cncnc2)c1 ; CC(C)(C)OC(=O)N1C[C@@H](C(=O)Nc2cccc(-c3cncnc3)c2)[C@H](c2ccccc2)C1 ; "1,4-dioxane" ; O=C(Nc1cccc(-c2cncnc2)c1)[C@@H]1CNC[C@H]1c1ccccc1]

### Step 1: tert-Butyl (±)-trans-4-phenyl-3-{[3-(pyrimidin-5-yl)phenyl]carbamoyl}pyrrolidine-1-carboxylate (116.1)

Intermediate 116.1 was synthesized according to the procedure described in Step 1 of Example 64 from intermediate 6.5 (200 mg, 0.69 mmol), HATU (313 mg, 0.82 mmol), DIPEA (359 μL, 2.06 mmol), intermediate 38.2 (176 mg, 1.03 mmol), and 3.0M EtMgBr in Et₂O (687 μL, 2.06 mmol) in THF (4 mL+4 mL). The intermediate 116.1 (64 mg, 0.14 mmol) was obtained after work-up and chromatographic purification (DCM/MeOH from 100% DCM to 95:5 v/v DCM/MeOH). Yield: 21%. MS-ESI(−) m/z: 443.0 (M−H); MS-ESI(+) m/z: 445.9 (M+H).

### Step 2: (±)-trans-4-Phenyl-N-[3-(pyrimidin-5-yl)phenyl]pyrrolidine-3-carboxamide trihydrochloride, (Compound I-53)

Compound I-53 was synthesized according to the procedure described in Step 2 of Example 64 from a solution of intermediate 116.1 (64 mg, 0.14 mmol) in 1,4-dioxane (1.2 mL) which was reacted with 4.0M HCl in 1,4-dioxane (360 μL). The title compound I-53 (61 mg, 0.07 mmol) was obtained as a yellow solid. Yield: 50%. ¹H-NMR (400 MHz, DMSO-d₆) δ 3.27-3.38 (m, 2H), 3.45-3.49 (m, 1H), 3.72-3.78 (m, 3H), 4.90 (brs, 1H), 7.27-7.47 (m, 9H), 7.66 (d, J=6.1 Hz, 1H), 7.94 (s, 1H), 9.02 (s, 1H), 9.19 (s, 1H), 9.53 (brs, 1H), 9.95 (brs, 1H), 10.61 (s, 1H). UHPLC purity: ≥95%. MS-ESI(+) m/z: 345.0 (M+H); MS-ESI(−) m/z: 342.9 (M−H).

### Example 117: (±)-trans-4-Phenyl-N-[3-(pyridin-3-yl)phenyl]-1-(tetrahydro-2H-pyran-4-yl)pyrrolidine-3-carboxamide (Compound I-54)

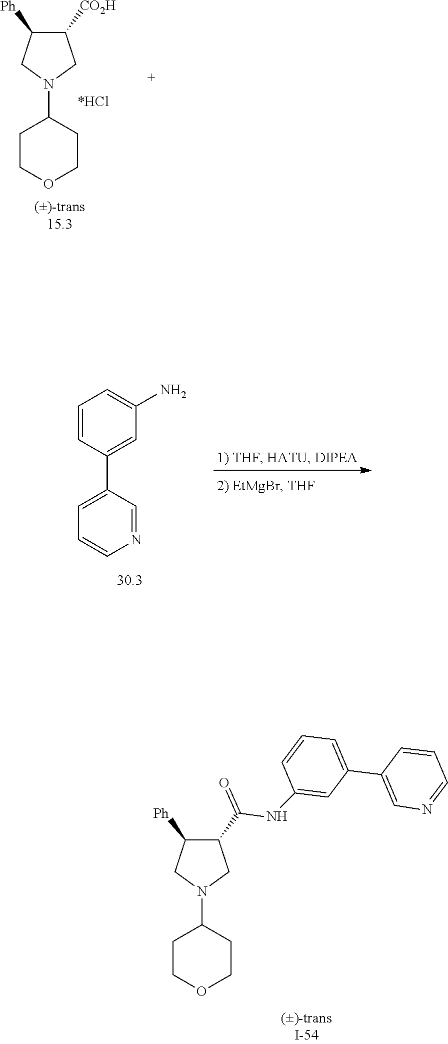 [SMILES: O=C(O)[C@@H]1CN(C2CCOCC2)C[C@H]1c1ccccc1 ; Nc1cccc(-c2cccnc2)c1 ; O=C(Nc1cccc(-c2cccnc2)c1)[C@@H]1CN(C2CCOCC2)C[C@H]1c1ccccc1]

Compound I-54 was synthesized according to the procedure described in Step 1 of Example 64 from intermediate 15.3 (150 mg, 0.48 mmol) which was reacted with HATU (274 mg, 0.72 mmol), DIPEA (294 μL, 1.68 mmol), intermediate 30.3 (123 mg, 0.0.72), and 3.0 M EtMgBr in Et₂O (0.48 mL, 1.44 mmol) in THF (3 mL+3 mL). the title compound I-54 (66 mg 0.15 mmol) was obtained after work-up and chromatographic purification (DCM/MeOH from 100% DCM to 94:6 v/v DCM/MeOH). Yield: 32%. ¹H-NMR (400 MHz, DMSO-d₆) δ 1.38 (brs, 2H), 1.75 (brs, 2H), 2.25-2.75 (m, 4H), 2.98-3.35 (m, 4H), 3.55-3.66 (m, 1H), 3.72-3.84 (m, 2H), 7.14-7.41 (m, 8H), 7.49 (d, J=7.3 Hz, 1H), 7.83 (s, 1H), 7.89 (d, J=8.2 Hz, 1H), 8.48 (d, J=3.6 Hz, 1H), 8.71 (s, 1H), 10.02 (brs, 1H). UHPLC purity ≥95%. MS-ESI(−) m/z: 426.8 (M−H); MS-ESI(+) m/z: 428.3 (M+H).

### Example 118: (±)-trans-1-Acetyl-4-phenyl-N-[3-(pyridin-3-yl)phenyl]pyrrolidine-3-carboxamide (Compound I-55)

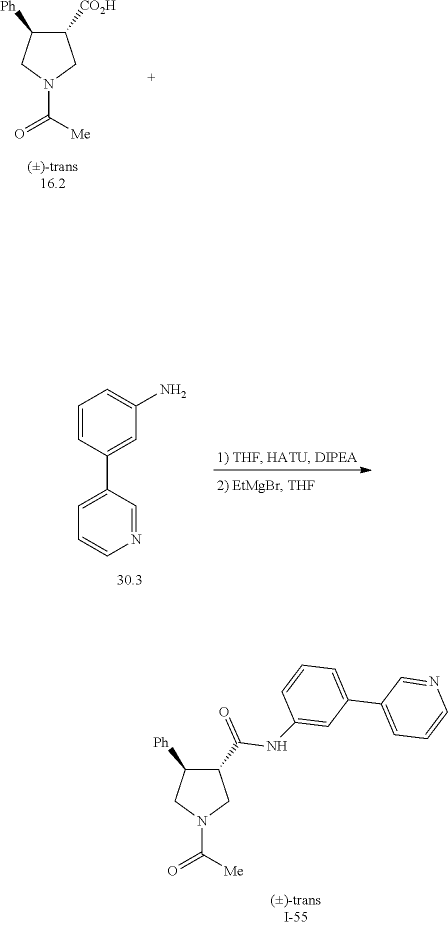 [SMILES: CC(=O)N1C[C@@H](C(=O)O)[C@H](c2ccccc2)C1 ; Nc1cccc(-c2cccnc2)c1 ; CC(=O)N1C[C@@H](C(=O)Nc2cccc(-c3cccnc3)c2)[C@H](c2ccccc2)C1]

Compound I-55 was synthesized according to the procedure described in Step 1 of Example 64 from intermediate 16.2 (150 mg, 0.64 mmol), HATU (367 mg, 0.96 mmol), DIPEA (337 μL, 0.1.93 mmol), intermediate 30.3 (164 mg, 0.96), and 3.0 M EtMgBr in Et₂O (0.64 μL, 1.93 mmol) in THF (3 mL+3 mL). The title compound I-55 (52 mg, 0.13 mmol) was obtained after flash chromatographic purification (DCM/MeOH from 99:1 to 95:5 v/v). Yield: 21%. ¹H-NMR (400 MHz, DMSO-d₆) δ 1.98 (s, 3H), 3.40-3.45 (m, 1H), 3.54-3.59 (m, 1H), 3.63-3.70 (m, 1H), 3.76-3.81 (m, 1H), 4.04-3.97 (m, 1H), 7.23-7.26 (m, 1H), 7.32-7.42 (m, 6H), 7.48-7.51 (m, 1H), 7.54-7.59 (m, 1H), 7.88 (s, 1H), 7.97-8.01 (m, 1H), 8.56-8.59 (m, 1H), 8.79 (s, 1H), 10.22 (s, 1H). UHPLC purity: ≥95%._MS-ESI(+) m/z: 385.9 (M+H); MS-ESI(−) m/z: 383.9 (M−H).

### Example 119: (3S,4S)—N-(Isoquinolin-5-yl)-4-(thiophen-2-yl)pyrrolidine-3-carboxamide (Compound I-56)

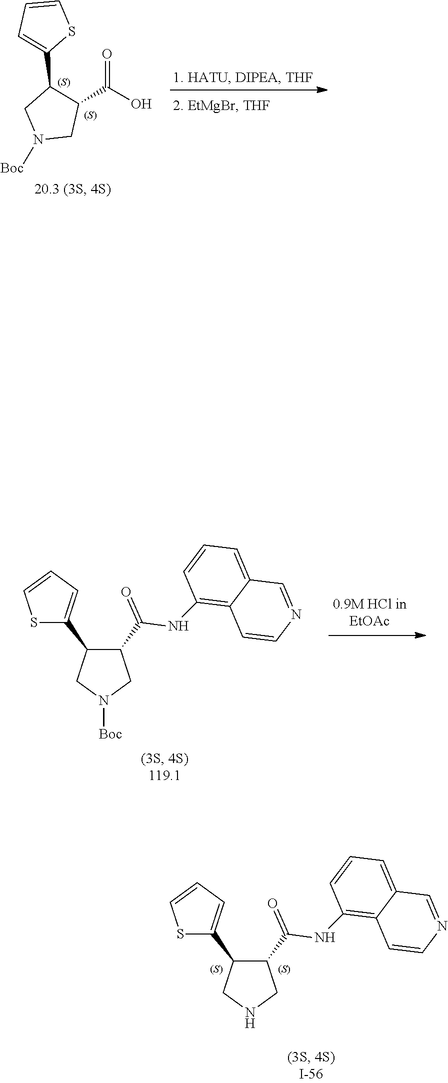

### Step 1: tert-Butyl (3S,4S)-4-(thiophen-2-yl)-3-(isoquinolin-5-ylcarbamoyl)pyrrolidine-1-carboxylate (119.1)

Intermediate 119.1 was synthesized according to the procedure described in Step 1 of Example 64 from intermediate 20.3 (300 mg, LOO mmol), HATU (575 mg, 1.51 mmol), DIPEA (530 μL, 3.02 mmol), intermediate 27.1 (218 mg, 1.51), and 3.0 M EtMgBr in Et₂O (1.00 mL, 3.02 mmol) in THF (5 mL+5 mL). The intermediate 119.1 (90 mg, 0.21 mmol) was obtained after chromatographic purification (DCM/MeOH from 100% DCM to 95:5 v/v DCM/MeOH). Yield: 21%. MS-ESI(+) m/z: 424.3 (M+H). MS-ESI(−) m/z: 422.3 (M−H);

### Step 2: (3S,4S)—N-(Isoquinolin-5-yl)-4-(thiophen-2-yl)pyrrolidine-3-carboxamide), (Compound I-56)

Intermediate 119.1 (90 mg, 0.21 mmol) was treated with 0.9M HCl in EtOAc (2.4 mL, 2.12 mmol) and the resulting mixture was reacted at r.t. for 16 h. The suspension was centrifuged, the supernatant was removed and the residue was washed with EtOAc (2×1 mL). Upon centrifugation and desiccation in a drying oven, the title compound I-56 (81 mg, 0.20 mmol) was obtained as a yellowish solid. Yield: 95%. UHPLC purity: ≥95%. MS-ESI(+) m/z: 324.2 (M+H). MS-ESI(−) m/z: 322.2 (M−H);

### Example 120: (3R,4S)—N-(Isoquinolin-5-yl)-N-methyl-4-phenylpyrrolidine-3-carboxamide dihydrochloride (Compound I-57)

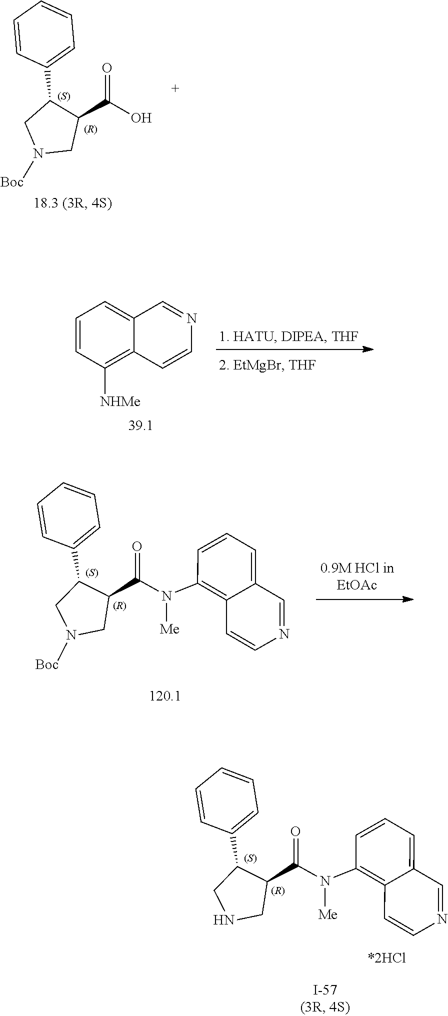

### Step 1: tert-Butyl (3R,4S)-4-phenyl-3-[isoquinolin-5-yl(methyl)carbamoyl]pyrrolidine-1-carboxylate(120.1)

Intermediate 120.1 was synthesized according to the procedure described in Step 1 of Example 33 from intermediate 18.3 (153 mg, 0.52 mmol), HATU (240 mg, 0.631 mmol), DIPEA (275 μL, 1.58 mmol), intermediate 39.1 (41 mg, 0.26), and 3.0 M EtMgBr in Et₂O (0.53 mL, 1.58 mmol) in THF (2 mL+2 mL). The intermediate 120.1 (35 mg, 0.08 mmol) was obtained after chromatographic purification (DCM/MeOH from 100% DCM to 95:5 v/v DCM/MeOH). Yield: 15%. MS-ESI(+) m/z: 432.3 (M+H).

### Step 2: 3R, 4S)—N-(Isoquinolin-5-yl)-N-methyl-4-phenylpyrrolidine-3-carboxamide dihydrochloride (Compound I-57)

Intermediate 120.1 (35 mg, 0.08 mmol) was treated with 0.9M HCl in EtOAc (901 μL, 0.81 mmol) and the resulting mixture was reacted at r.t. for 16 h. The suspension was centrifuged, the supernatant was removed and the residue was washed with EtOAc (2×1 mL). Upon centrifugation and desiccation in drying oven, the title compound I-57 (28 mg, 0.07 mmol) was obtained as a yellowish solid. Yield: 88%. ¹H-NMR (400 MHz, DMSO-d₆) δ 3.30-3.36 (m, 1H), 3.47 (s, 3H), 3.50-3.79 (m, 5H), 6.05 (d, J=7.6 Hz), 1H), 7.34-7.68 (m, 7H), 7.69 (d, J=7.7 Hz, 1H), 8.05 (d, J=8.0 Hz, 1H), 9.41 (brs, 1H), 9.70 (brs, 1H), 10.04 (s, 1H). UHPLC purity: ≥95%. MS-ESI(+) m/z: 348.4 (M+H). MS-ESI(−) m/z: 346.3 (M−H);

### Example 121: (3R,4R)—N-(Isoquinolin-5-yl)-4-(1,3-thiazol-2-yl)pyrrolidine-3-carboxamide dihydrochloride (Compound I-58)

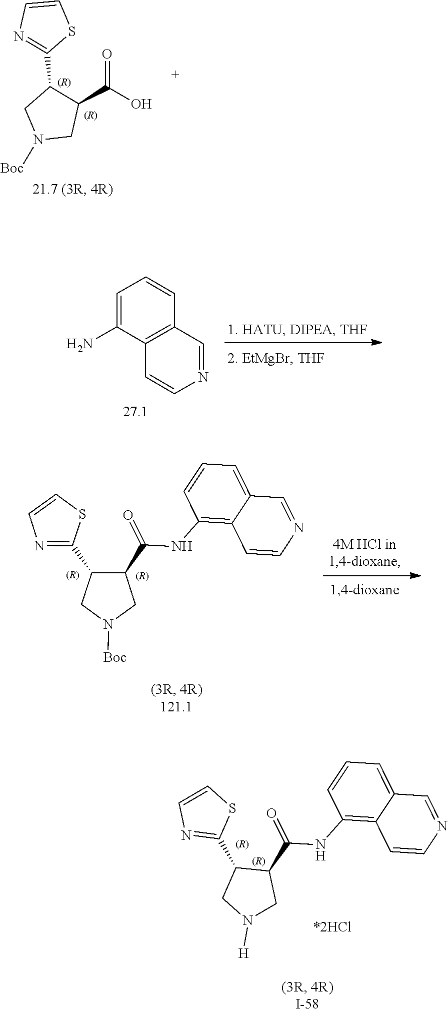

### Step 1: tert-Butyl (3R,4R)-4-(1,3-thiazol-2-yl)-3-(isoquinolin-5-ylcarbamoyl)pyrrolidine-1-carboxylate (121.1)

Intermediate 121.1 was prepared according to the procedure described in Step 1 of Example 64 starting from a solution of intermediate 21.7 (300 mg, 0.95 mmol), HATU (472 mg, 1.24 mmol), DIPEA (0.49 mL, 2.85 mmol), 3.0 M EtMgBr in Et₂O (0.95 mL, 2.85 mmol), and intermediate 27.1 (205 mg, 1.42 mmol) in THF (5 mL+5 mL). Stirring was continued at r.t. for 16 h. After purification by flash chromatography (DCM/MeOH from 100% DCM to 97:3 v/v DCM/MeOH), the intermediate 121.1 (150 mg, 0.35 mmol) was obtained as a colorless oil. Yield 37%.

### Step 2: (3R,4R)—N-(Isoquinolin-5-yl)-4-(1,3-thiazol-2-yl)pyrrolidine-3-carboxamide dihydrochloride, (Compound I-58)

Compound I-58 was prepared following the procedure described in Step 2 of Example 64 starting from a solution of intermediate 121.1 (60 mg, 0.14 mmol) in 1,4-dioxane (5 mL) and 1M HCl in EtOAc (1.4 mL, 1.4 mmol). Stirring was continued at r.t. for 16 h. The title compound I-58 (30 mg, 0.07 mmol) was obtained as the dihydrochloride salt as white powder after trituration with Et₂O. Yield: 54%. ¹H-NMR (400 MHz, DMSO-d₆) δ 3.49 (m, 1H), 3.59 (m, 1H), 3.87 (m, 3H), 4.30 (d, J=7.9 Hz, 1H), 7.76 (m, 1H), 7.87 (m, 1H), 8.02 (t, J=7.1 Hz, 1H), 8.33 (d, J=7.1 Hz, 1H), 8.40 (d, J=7.7 Hz, 1H), 8.51-8.56 (m, 1H), 8.73 (d, J=7.5 Hz, 1H), 9.9 (s, 2H), 10.13 (brs, 1H), 11.1 (s, 1H). HPLC purity: >95%. MS-ESI(+) m/z: 325.3 (M+H); MS-ESI(−) m/z: 323.2 (M−H).

### Example 122: (3S,4S)—N-(Isoquinolin-5-yl)-4-(1,3-thiazol-2-yl)pyrrolidine-3-carboxamide dihydrochloride (1-59)

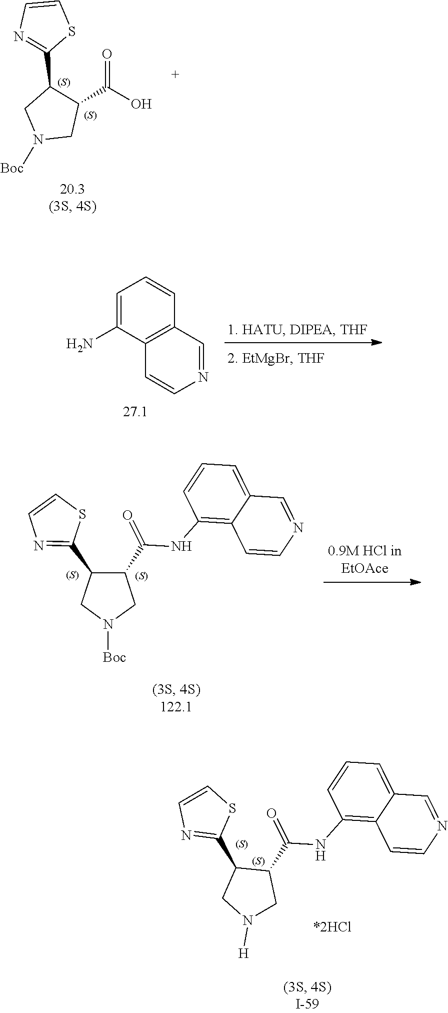

### Step 1: tert-Butyl (3S,4S)-4-(1,3-thiazol-2-yl)-3-(isoquinolin-5-ylcarbamoyl)pyrrolidine-1-carboxylate (122.1)

Intermediate 122.1 was prepared according to the procedure described in Step 1 of Example 64 starting from a solution of intermediate 20.3 (300 mg, 0.95 mmol), HATU (472 mg, 1.24 mmol), DIPEA (0.49 mL, 2.85 mmol), 3.0 M EtMgBr in Et₂O (0.95 mL, 2.85 mmol), and intermediate 27.1 (205 mg, 1.42 mmol) in THF (5 mL+5 mL). Stirring was continued at r.t. 16 h. Purification by flash chromatography (DCM/MeOH from 100% DCM to 97:3 v/v DCM/MeOH) the intermediate 122.1 (80 mg, 0.18 mmol) was obtained as a colorless oil. Yield 20%.

### Step 2: (3S,4S)—N-(Isoquinolin-5-yl)-4-(1,3-thiazol-2-yl)pyrrolidine-3-carboxamide dihydrochloride, (Compound I-59)

Compound I-59 was prepared following the procedure described in Step 2 of Example 45 starting from a solution of intermediate 122.1 (80 mg, 0.18 mmol) in 1,4-dioxane (5 mL) and 0.9 M HCl in EtOAc (1.8 mL). Stirring was continued at r.t. for 16 h. The title compound I-59 (25 mg, 0.06 mmol) as a dihydrochloride salt was obtained as a white powder after trituration with Et₂O. Yield: 35%. ¹H-NMR (400 MHz, DMSO-d₆) δ 3.48 (s, 1H), 3.58 (s, 1H), 3.85 (m, 3H), 4.29 (m, 1H), 7.49 (m, 1H), 7.74 (s, 1H), 7.85 (s, 1H), 8.01 (s, 1H), 8.31-8.38 (m, 2H), 8.51 (m, 1H), 8.73 (m, 1H), 9.92 (brs, 2H), 10.08 (brs, 1H), 11.1 (s, 1H). HPLC purity: >95%. MS-ESI(+) m/z: 325.3 (M+H); MS-ESI(−) m/z: 323.1 (M−H).

### Example 123: 5-{[(3R,4S)-4-Phenylpyrrolidin-3-yl]methoxy}isoquinoline dihydrochloride (Compound I-60)

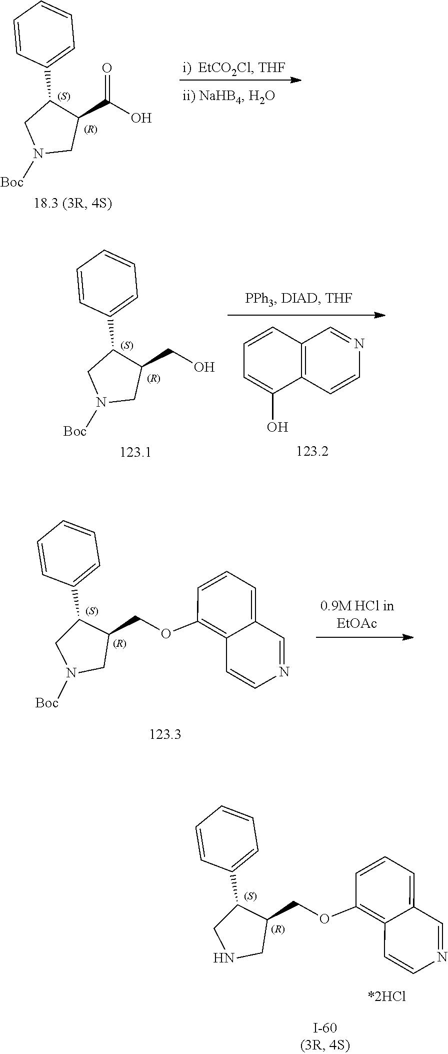

### Step 1: tert-Butyl (3R,4S)-3-(hydroxymethyl)-4-phenylpyrrolidine-1-carboxylate (123.1)

Et₃N (0.19 mL, 1.33 mmol) and EtCO₂Cl (0.11 mL, 1.13 mmol) were added to a solution of intermediate 18.3 (300 mg, 1.029 mmol) in THF (5 mL) and stirring was continued at r. t. 3 h. A suspension of NaBH₄ in H₂O was then added dropwise to the mixture and stirring was continued at r. t. additional 16 h. The crude was diluted with water and extracted with EtOAc (3×30 mL). The organic phases were collected together, washed with brine, and dried over anhydrous Na₂SO₄. Purification by flash chromatography (DCM/MeOH from 100% DCM to 97:3 v/v DCM/MeOH) the intermediate 123.1 (150 mg, 0.54 mmol) was obtained as a colorless oil. Yield 50%.

### Step 2: tert-Butyl (3R,4S)-3-(isoquinolin-5-yloxy-methyl)-4-phenylpyrrolidine-1-carboxylate (123.3)

To a solution of intermediate 123.1 (100 mg, 0.36 mmol) in THF (5 mL) was added PPh₃ (209 mg, 0.79 mmol), DIAD (0.14 mL, 0.72 mmol), and intermediate 123.2 (78.3 mg, 0.54 mmol). Stirring was continued at r.t. for 16 h. The crude was diluted with water and was extracted with EtOAc (3×30 mL). The organic phases were collected together, washed with brine and dried over anhydrous Na₂SO₄. After purification by flash chromatography (DCM/MeOH from 100% DCM to 97:3 v/v DCM/MeOH) the intermediate 123.3 (75 mg, 0.19 mmol) was obtained as a white solid. Yield 51%.

### Step 3: 5-{[(3R,4S)-4-Phenylpyrrolidin-3-yl]methoxy}isoquinoline dihydrochloride (Compound I-60)

Intermediate 123.3 (44 mg, 0.11 mmol) was treated with a 0.9M solution of HCl in EtOAc (1.21 mL; 1.1 mmol) and the resulting mixture was reacted at r.t. for 16 h. The suspension was centrifuged, the supernatant was removed and the residue was washed with EtOAc (2×1 mL). Upon centrifugation and desiccation in drying oven, the title compound I-60 (30 mg, 0.07 mmol) was obtained as a white solid. Yield: 80%. ¹H-NMR (400 MHz, DMSO-de) δ 3.03 (s, m, 1H), 3.25-3.34 (m, 2H), 3.44-3.54 (m, 1H), 3.70 (m, 2H), 4.28 (d, J=8 Hz, 2H), 7.28-7.37 (m, 4H), 7.43 (d, J=8 Hz, 2H), 7.54 (d, J=8 Hz, 1H), 7.86 (t, J=8 Hz, 1H), 8.02 (d, J=8 Hz, 1H), 8.17 (d, J=4 Hz, 1H), 8.55 (d, J=4 Hz, 1H), 9.82 (s, 1H), 10.04 (m, 2H). HPLC purity: >95%. MS-ESI(+) m/z: 325.3 (M+H); MS-ESI(−) m/z: 323.1 (M−H).

### Example 124: 5-{[(±)-cis-4-Phenylpyrrolidin-3-yl]oxy}isoquinoline dihydrochloride (Compound I-61)

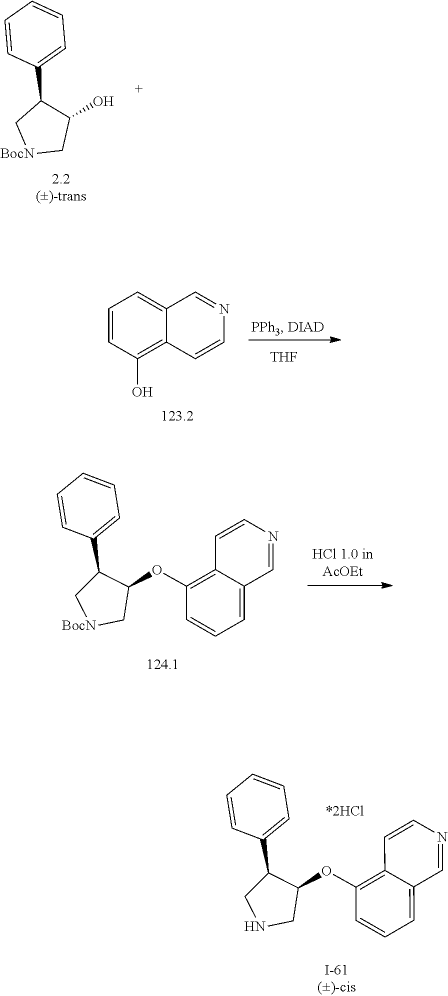 [SMILES: CC(C)(C)OC(=O)N1C[C@@H](O)[C@H](c2ccccc2)C1 ; Oc1cccc2cnccc12 ; CC(C)(C)OC(=O)N1C[C@H](Oc2cccc3cnccc23)[C@H](c2ccccc2)C1 ; "THF" ; c1ccc([C@@H]2CNC[C@@H]2Oc2cccc3cnccc23)cc1]

### Step 1: tert-Butyl (±)-cis-3-(isoquinolin-5-yloxy)-4-phenylpyrrolidine-1-carboxylate (124.1)

Intermediate 124.1 was prepared following the procedure described in Step 2 of Example 123 starting from a solution of intermediate 2.2 (150 mg, 0.57 mmol) which was reacted with PPh₃ (329 mg, 1.25 mmol), DIAD (0.22 mL, 1.14 mmol), and intermediate 123.2 (124 mg, 0.85 mmol) in THF (7.5 mL). After purification by flash chromatography (DCM/MeOH from 100% DCM to 97:3 v/v DCM/MeOH) the intermediate 124.1 (50 mg, 0.13 mmol) was obtained as a white solid. Yield: 22%. MS-ESI(+) m/z: 391.5 (M+H).

### Step 2: 5-{[(±)-cis-4-Phenylpyrrolidin-3-yl]oxy}isoquinoline dihydrochloride (Compound I-61)

Compound I-61 was prepared following the procedure described in Step 2 of Example 120 starting from a solution of intermediate 124.1 (46 mg, 0.12 mmol) and a 0.9 M solution of HCl in EtOAc (1.30 mL). The title compound I-61 (24 mg, 0.07 mmol) was obtained as a pale yellow solid. Yield 56%. (400 MHz, DMSO-d₆) δ 3.58-3.63 (m, 1H), 3.78-3.93 (m, 4H), 5.63-5.65 (m, 1H), 7.17-7.27 (m, 3H), 7.47 (d, J=7.58 Hz, 2H), 7.53 (d, J=7.9 Hz, 1H), 7.80 (t, J=8.1 Hz, 1H), 7.99 (d, J=8.2 Hz, 1H), 8.43 (d, J=6.5 Hz, 1H), 8.61 (d, J=6.5 Hz, 1H), 9.78 (s, 1H), 10.16 (brs, 2H). HPLC purity: ≥95%. MS-ESI(+) m/z: 291.3 (M+H).

### Example 125: (±)-trans-1,4-Diphenyl-N-[3-(pyridin-3-yl)phenyl]pyrrolidine-3-carboxamide (Compound I-62)

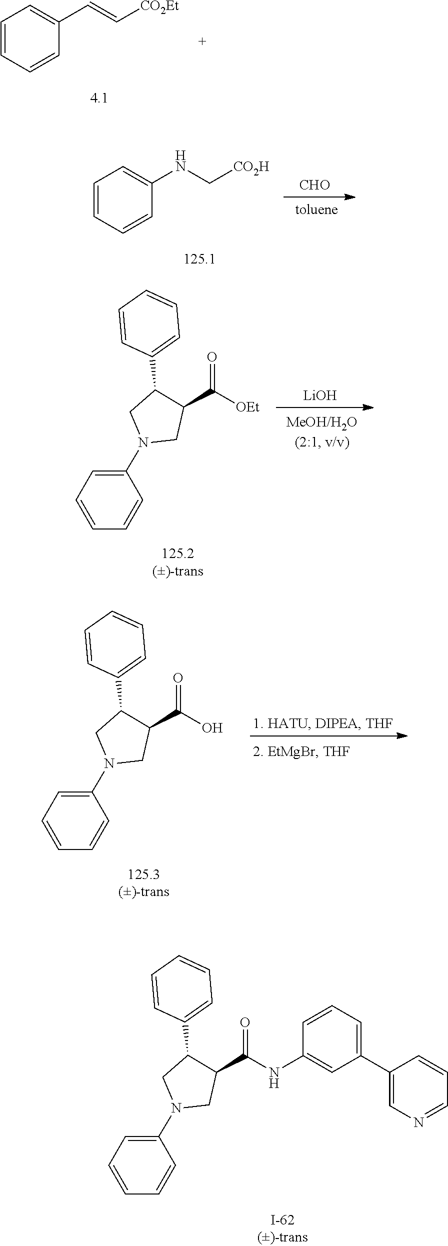

### Step 1: Ethyl (±)-trans-1,4-diphenylpyrrolidine-3-carboxylate (125.2)

p-Formaldehyde (1.09 g, 36.32 mmol) and N-phenylglycine, intermediate 125.1, (1.89 mg, 12.48 mmol) were added to a solution of intermediate 4.1 (2.0 g, 11.35 mmol) in toluene (40 mL) and stirring was continued at reflux with a Dean Stark apparatus for 16 h. The solvent was removed under vacuo. After purification by flash chromatography (PET/EtOAc from 100% PET to 95:5 v/v PET/EtOAc) the intermediate 125.2 (408 mg, 1.38 mmol) was obtained as a colorless sticky oil. Yield: 12%. MS-ESI(+) m/z: 296.4 (M+H).

### Step 2: (±)-trans-1,4-Diphenylpyrrolidine-3-carboxylic acid (125.3)

LiOH (165 mg, 6.91 mmol) was added to a solution of intermediate 125.2 (408 mg, 1.38 mmol) in MeOH/H₂O (2:1, v/v, 6 mL) and the resulting mixture was reacted at r.t. for 2 h. The solvent was removed under vacuo, the residue was taken up with H₂O (30 mL) and washed with EtOAc (2×20 mL). The aqueous layer was acidified with 0.5M aq. citric acid up to pH=3, then extracted with EtOAc (3×20 mL). The combined organic phases were washed with H₂O (30 mL) and brine (30 mL), dried over anhydrous Na₂SO₄, and evaporated under vacuum. After purification by flash chromatography (DCM/MeOH from 100% DCM to 95:5 v/v DCM/MeOH) the intermediate 125.3 (256 mg, 0.96 mmol) was obtained as a colorless vitreous solid. Yield: 69%. MS-ESI(−) m/z: 266.5 (M−H).

### Step 3: (H-trans-1,4-Diphenyl-N-[3-(pyridin-3-yl)phenyl]pyrrolidine-3-carboxamide (I-62)

Compound I-62 was prepared according to the procedure described in Step 1 of Example 64 starting from intermediate 125.3 (125 mg, 0.47 mmol) which was reacted with HATU (213 mg, 0.56 mmol), DIPEA (0.24 mL, 1.40 mmol), 3.0 M EtMgBr in Et₂O (0.47 mL, 1.40 mmol), and intermediate 23.1 (129 mg, 0.70 mmol) in THF (2.5 mL+2.5 mL). Stirring was continued at r.t. for 16 h. After purification by flash chromatography (PET/EtOAc from 9:1 v/v to 1:1 PET/EtOAc) the title compound I-62 (47 mg, 0.11 mmol) was obtained as a white solid. Yield 24%. ¹H-NMR (400 MHz, DMSO-d₆) δ 3.39 (t, J=8.5 Hz, 1H), 3.49-3.52 (m, 2H), 3.80-3.91 (m, 3H), 6.60-6.66 (m, 3H), 7.17-7.27 (m, 3H), 7.33-7.51 (m, 7H), 7.61 (d, J=7.6 Hz, 1H), 7.93 (s, 1H), 7.99 (d, J=8.0 Hz, 1H), 8.58 (d, J=4.0 Hz, 1H), 8.81 (s, 1H), 10.25 (s, 1H). HPLC purity: ≥95%. MS-ESI(+) m/z: 420.5 (M+H).

### Example 126: (±)-trans-1-Benzyl-4-phenyl-N-[3-(pyridin-3-yl)phenyl]pyrrolidine-3-carboxamide (Compound I-63)

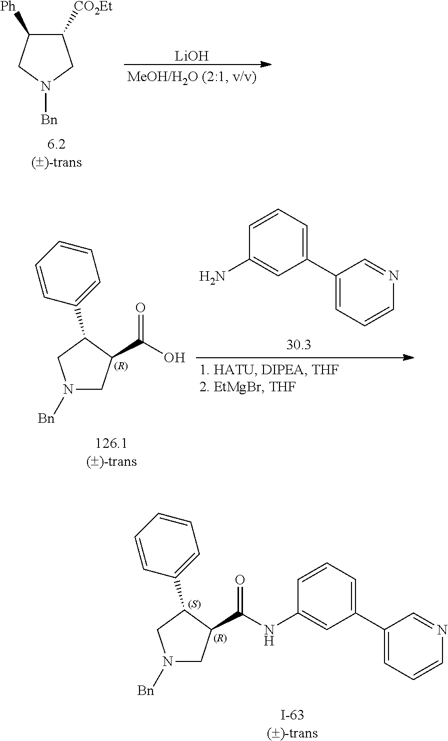

### Step 1: Ethyl (H-trans I-benzyl-4-phenylpyrrolidine-3-carboxylate (126.1)

Compound 126.1 was prepared according to the procedure described in Step 3 of Example 6 starting from intermediate 6.2 (765 mg, 2.47 mmol) which was reacted with LiOH (296 mg, 12.36 mmol) in MeOH/H₂O (2:1, v/v, 10.5 mL). Stirring was continued at r.t. for 2 h. The intermediate 126.1 (145 mg, 0.52 mmol) was obtained as a colorless oil. Yield 21%. MS-ESI(−) m/z: 280.4 (M−H).

### Step 2: (±)-trans-1-Benzyl-4-phenyl-N-[3-(pyridin-3-yl)phenyl]pyrrolidine-3-carboxamide (F63)

Compound I-63 was prepared according to the procedure described in Step 1 of Example 64 starting from a solution of intermediate 126.1 (143 mg, 0.51 mmol), HATU (231 mg, 0.61 mmol), DIPEA (0.27 mL, 1.52 mmol), 3.0 M EtMgBr in Et₂O (0.51 mL, 1.52 mmol), and intermediate 30.3 (140 mg, 0.76 mmol) in THF (2.5 mL+2.5 mL). Stirring was continued at r.t. for 16 h. After purification by flash chromatography (PET/EtOAc from 9:1 v/v to 1:1 PET/EtOAc) the title compound I-63 (44 mg, 0.11 mmol) was obtained as a yellow solid. Yield 20%. (400 MHz, DMSO-d₆) δ 3.60-3.87 (m, 4H), 4.06-4.10 (m, 1H), 4.49-4.58 (m, 3H), 7.32-7.64 (m, 14H), 7.88 (s, 1H), 8.11-8.15 (m, 1H), 8.64-8.66 (m, 1H), 1.14 (s, 1H), 10.25-10.28 (m, 1H), 10.42 (brs, 1H). HPLC purity: ≥90%. MS-ESI(+) m/z: 434.6 (M+H).

### Example 127: 5-{[(±)-trans-4-Phenylpyrrolidin-3-yl]oxy}isoquinoline dihydrochloride (Compound I-64)

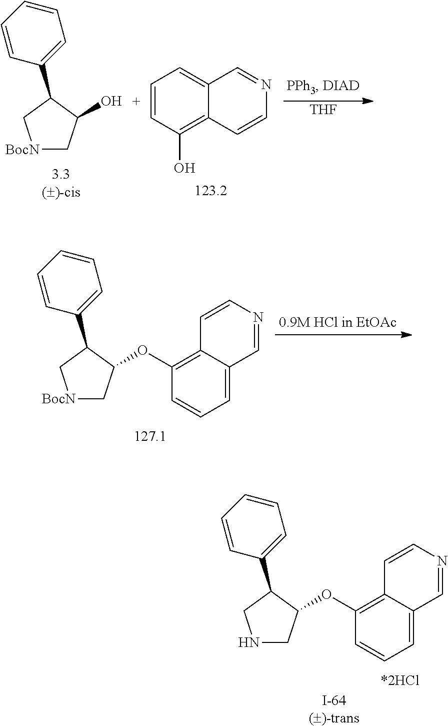

### Step 1: tert-Butyl (±)-trans-3-(isoquinolin-5-yloxy)-4-phenylpyrrolidine-1-carboxylate (127.1)

Intermediate 127.1 was prepared following the procedure described in Step 2 of Example 123 starting from a solution of intermediate 3.3 (181 mg, 0.69 mmol) which was reacted with PPh₃ (395 mg, 1.51 mmol), DIAD (0.27 mL, 1.38 mmol), and 5-hydroxyisoquinoline, intermediate 123.2 (150 mg, 1.03 mmol) in THF (10 mL). After purification by flash chromatography (DCM/MeOH from 100% DCM to 97:3 v/v DCM/MeOH) the intermediate 127.1 (8.7 mg, 0.02 mmol) was obtained. Yield: 3.2%. MS-ESI(+) m/z: 391.3 (M+H).

### Step 2: 5-{[(±)-trans-4-Phenylpyrrolidin-3-yl]oxy}isoquinoline dihydrochloride (Compound I-64)

Compound I-64 was prepared following the procedure described in Step 2 of Example 120 starting from a solution of intermediate 127.1 (8.7 mg, 0.022 mmol) and a 0.9 M solution of HCl in EtOAc (0.28 mL). The title compound I-64 (5.7 mg, 0.020 mmol) was obtained as a white solid. Yield: 91%. (400 MHz, DMSO-d₆) δ 3.52-3.75 (m, 2H), 3.84-3.95 (m, 3H), 5.47 (s, 1H), 7.36-7.38 (m, 1H), 7.42-7.46 (m, 2H), 7.50-7.54 (m, 3H), 7.86 (t, J=8.1 Hz, 1H), 8.04 (d, J=8.3 Hz, 1H), 8.64 (d, J=6.1 Hz, 1H), 8.71 (d, J=6.3 Hz), 9.76 (s, 1H), 10.07 (brs, 1H), 10.23 (brs, 1H).

HPLC purity: ≥95%. MS-ESI(+) m/z: 291.2 (M+H).

### Example 128: (±)-trans-N-(2,3-Dihydro-1,4-benzodioxin-5-yl)-4-phenylpyrrolidine-3-carboxamide hydrochloride (Compound I-65)

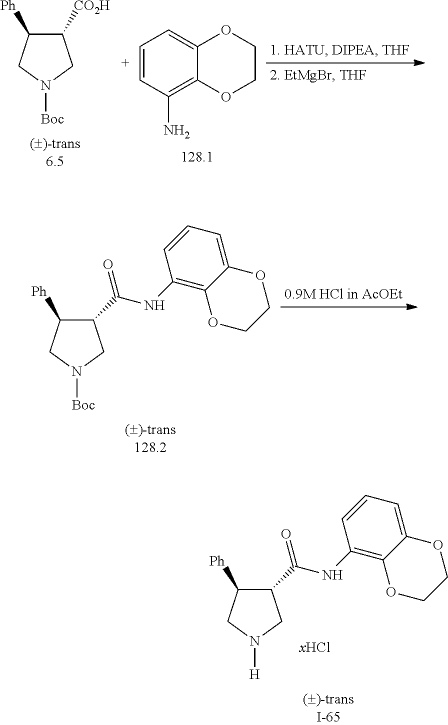 [SMILES: CC(C)(C)OC(=O)N1C[C@@H](C(=O)O)[C@H](c2ccccc2)C1 ; Nc1cccc2c1OCCO2 ; CC(C)(C)OC(=O)N1C[C@@H](C(=O)Nc2cccc3c2OCCO3)[C@H](c2ccccc2)C1 ; O=C(Nc1cccc2c1OCCO2)[C@@H]1CNC[C@H]1c1ccccc1]

### Step 1: tert-Butyl (±)-trans-3-(2,3-dihydro-1,4-benzodioxin-5-ylcarbamoyl)-4-phenylpyrrolidine-1-carboxylate (128.2)

Intermediate 128.2 was prepared according to the procedure described in Step 1 of Example 64 starting from intermediate 6.5 (200 mg, 0.69 mmol), HATU (313 mg, 0.82 mmol), DIPEA (0.36 mL, 2.06 mmol), 3.0 M EtMgBr in Et₂O (0.69 mL, 2.06 mmol), and intermediate 128.1 (0.12 mL, 1.03 mmol) in THF (3.5 mL+3.5 mL). Stirring was continued at r.t. for 16 h. After purification by flash chromatography (DCM/MeOH from 100% DCM to 95:5 v/v DCM/MeOH) the intermediate 128.2 (86 mg, 0.20 mmol) was obtained as a yellowish solid. Yield 30%. MS-ESI(+) m/z: 425.6 (M+H).

### Step 2: (±)-trans-N-(2,3-Dihydro-1,4-benzodioxin-5-yl)-4-phenylpyrrolidine-3-carboxamide hydrochloride (Compound I-65)

Compound I-65 was prepared following the procedure described in Step 2 of Example 120 starting from a solution of intermediate 128.2 (86 mg, 0.20 mmol) and a 0.9 M solution of HCl in EtOAc (2.2 mL). The title compound I-65 (64 mg, 0.18 mmol) was obtained as a white solid. Yield: 88%. (400 MHz, DMSO-d₆) δ 3.25-3.29 (m, 2H), 3.60-3.72 (m, 4H), 4.03-4.20 (m, 4H), 6.62 (d, J=8.2 Hz, 1H), 6.73 (t, J=8.1 Hz, 1H), 7.28-7.39 (m, 6H), 9.34 (s, 1H), 9.45 (brs, 1H), 9.76 (brs, 1H). HPLC purity: ≥95%. MS-ESI(+) m/z: 325.3 (M+H).

### Example 129: (3S,4S)—N-(Isoquinolin-5-yl)-4-phenylpyrrolidine-3-carboxamide dihydrochloride (Compound I-65)

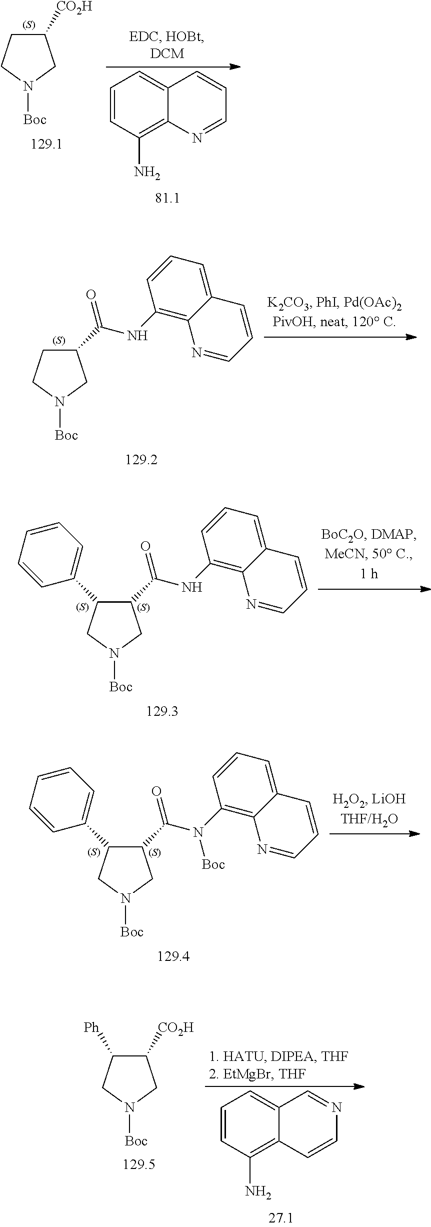

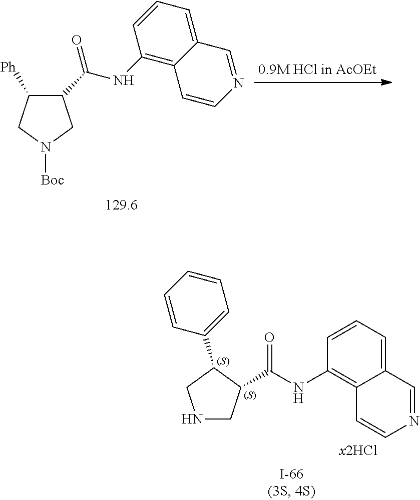

### Step 1: tert-Butyl (3S)-3-(quinolin-8-ylcarbamoyl)pyrrolidine-1-carboxylate (129.2)

HOBt (430 mg, 2.8 mmol) was added to a solution of intermediate 129.1 (500 mg, 2.33 mmol) and intermediate 81.1 (404 mg, 2.8 mmol) in dry DCM (9 mL). Stirring was continued at r.t. 5 min. EDC (537 mg, 2.8 mmol) was added to the mixture and stirring was continued at r.t. additional 72 h. The crude was poured into water, diluted with DCM (50 mL) and washed with water (50 mL), 1 M solution of HCl (30 mL), NaHCO₃ (ss) (50 mL) and brine (60 mL). Purification by flash chromatography (PET/EtOAc from 100% PET to 60:40 v/v PET/EtOAc) gave the intermediate 129.2 (715 mg, 2.09 mmol) as a colorless oil. Yield 90%.

MS-ESI(+) m/z: 342.2 (M+H).

### Step 2: tert-Butyl (3S,4S)-4-phenyl-N-(quinolin-8-yl)pyrrolidine-3-carboxamide-1-carboxylate (129.3)

K₂CO₃ (40.4 mg, 0.29 mmol) was flamed in a dry vial. Then the vial was charged under argon with Pd(OAc)₂ (3.25 mg, 0.014 mmol), PivOH (30 mg, 0.29 mmol), and intermediate 129.2 (100 mg, 0.29 mmol). The reaction vessel was sealed and purged with argon 3 times. Iodobenzene (0.16 mL, 0.87 mmol) was then added and the mixture was sonicated for 5 min under argon. The reaction tube was then placed in a preheated bath and stirred at 120° C. for 48 h. The reaction was allowed to cool to r.t., diluted with EtOAc (10 mL) and filtered through a short pad of celite. Purification by flash chromatography (DCM/MeCN from 100% DCM to 10:90 v/v DCM/MeCN) gave the intermediate 129.3 (55 mg, 0.13 mmol) as a colorless oil. Yield 45%.

MS-ESI(+) m/z: 418.3 (M+H); MS-ESI(−) m/z: 416.3 (M−H).

### Step 3: (3S,4S)-1-Pyrrolidinecarboxylic acid, 3-[[[(1,1-dimethylethoxy)carbonyl]-8-quinolinylamino]carbonyl]-4-(4-phenyl)-,1,1-dimethylethyl ester, (129.4)

A flame dried reaction tube was charged with a solution of intermediate 129.3 (100 mg, 0.32 mmol), Boc2O (0.36 mL, 1.57 mmol), and DMAP (83 mg, 0.64 mmol) in MeCN (0.7 mL). The reaction was heated at 50° C. for 2 h, then allowed to cool to r.t., before the tube was gently open and the reaction quenched with NH4⁺Cl⁻. The mixture was diluted with DCM (10 mL) and water (15 mL) and the crude was diluted with water then extracted with DCM (3×30 mL). The organic phase were collected together, washed with brine and dried over anhydrous Na₂SO₄. Purification (PET/Acetone from 100% PET to 80:20 v/v PET/Acetone) the intermediate 129.4 (132 mg, 0.25 mmol) was obtained as a white solid. Yield 80%.

MS-ESI(+) m/z: 518.5 (M+H).

### Step 4: (3S,4S)-1-(tert-Butoxycarbonyl)-4-phenylpyrrolidine-3-carboxylic acid (129.5)

A solution of 30% aq. H₂O₂ (60 μL, 0.58 mmol) in THF (600 μL) was added under argon at 0° C. to a solution of LiOH (10 mg, 0.38 mmol) in water (600 μL). The mixture was added dropwise under argon to a solution of intermediate 129.4 (100 mg, 0.19 mmol) in THF (800 μL). The vial was sealed and stirring was continued at r.t. 2 h. The reaction was quenched by addition of Na₂S₂O₃ (sat) and the aqueous phase was extracted with EtOAc (3×20 mL). The pH was brought to 2 by the addition of 1M HCl solution and the aqueous phase was extracted with EtOAc (3×30 mL). The combined organic phase was washed with brine and dried over anhydrous Na₂S04 to afford the intermediate 129.5 (50 mg, 0.19 mmol) as a white solid. Yield 89%.

MS-ESI(−) m/z 290.1 (M−H).

### Step 5: tert-Butyl (3S,4S)—N-(isoquinolin-5-yl)-4-phenylpyrrolidine-3-carboxamide-1-carboxylate (129.6)

Intermediate 129.6 was prepared according to the procedure described in Step 1 of Example 64 starting from a solution of intermediate 129.5 (230 mg, 0.79 mmol), HATU (391 mg, 1.026 mmol), DIPEA (0.21 mL, 1.19 mmol), 3.0M EtMgBr in Et₂O (0.8 mL, 2.37 mmol), and intermediate 27.1 (171 mg, 1.85 mmol) in THF (5 mL+5 mL). Stirring was continued at r.t. for 16 h. After purification by flash chromatography (DCM/MeOH from 100% DCM to 96.5:3.5 v/v DCM/MeOH), the intermediate 129.6 (80 mg, 0.19 mmol) was obtained as a colorless oil. Yield 24%.

MS-ESI(+) m/z: 418.3 (M+H); MS-ESI(−) m/z: 416.3 (M−H).

### Step 6: (3S,4S)—N-(Isoquinolin-5-yl)-4-phenylpyrrolidine-3-carboxamide dihydrochloride (Compound I-66)

Compound I-66 was prepared following the procedure described in Step 2 of Example 120 starting from a solution of intermediate 129.6 (80 mg, 0.19 mmol) and a 0.9 M solution of HCl in EtOAc (2.12 mL). The title compound I-66 (50 mg, 0.13 mmol) was obtained as a white solid. Yield: 66%. ¹H-NMR (400 MHz, DMSO-d₆) δ 3.66 (m, 6H), 4.02 (dd, J=9.4, 4.2 Hz, 4H), 7.26 (d, J=6.7 Hz, 1H), 7.31 (t, J=7.3 Hz, 2H), 7.39 (d, J=7.5 Hz, 2H), 7.63 (d, J=6.6 Hz, 1H), 7.72 (d, J=7.6 Hz, 1H), 7.83 (t, J=7.8 Hz, 1H), 8.21 (d, J=8.2 Hz, 1H), 8.50 (d, J=6.5 Hz, 1H), 9.54 (brs, 1H), 9.72 (s, 1H), 9.90 (brs, 1H), 10.62 (s, 1H). HPLC purity: ≥90%. MS-ESI(+) m/z: 318.3 (M+H).

### Example 130: (3R,4S)—N-(Isoquinolin-5-ylmethyl)-4-phenylpyrrolidine-3-carboxamide dihydrochloride (Compound I-67)

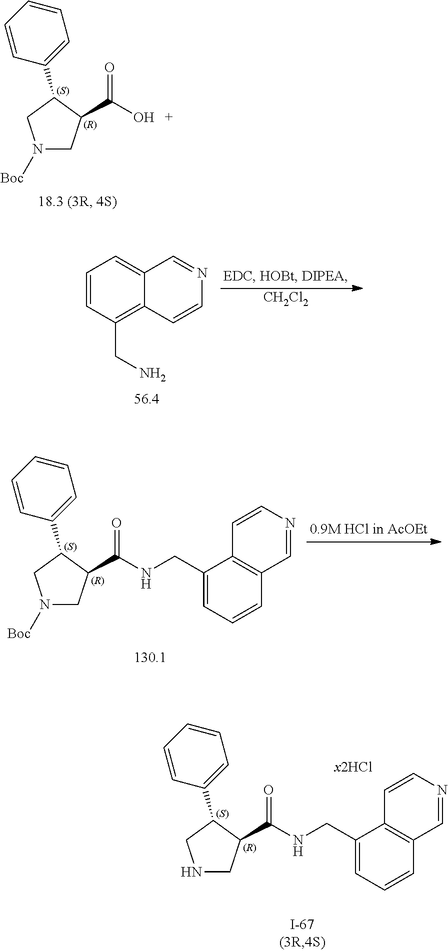

### Step 1: tert-Butyl (3R,4S)—N-(isoquinolin-5-ylmethyl)-4-phenylpyrrolidine-3-carboxamide-1-carboxylate (130.1)

To a solution of intermediate 18.3 (200 mg, 0.69 mmol) in THF (10 mL), was added DIPEA (0.36 mL, 20.7 mmol) and HATU (339.3 mg, 0.89 mmol). Stirring was then continued at r.t. 1 h. Intermediate 56.4 (131 mg, 0.83 mmol) was added to the mixture and stirring was continued at r.t. for a further 16 h. The crude was diluted with EtOAc (50 mL) and washed sequentially with a 0.5 M solution of citric acid (30 mL), water (30 mL), and brine.

Purification by flash chromatography (DCM/MeOH from 100% DCM to 97:3 v/v DCM/MeOH) gave the intermediate 130.1 (180 mg, 0.41 mmol) as a white solid. Yield 60%.

### Step 2: (3R,4S)—N-(Isoquinolin-5-ylmethyl)-4-phenylpyrrolidine-3-carboxamide dihydrochloride (Compound I-67)

Compound I-67 was prepared following the procedure described in Step 2 of Example 120 starting from a solution of intermediate 130.1 (100 mg, 0.23 mmol) and a 0.9 M solution of HCl in EtOAc (2.6 mL). The title compound I-67 (80 mg, 0.19 mmol) was obtained as a white solid. Yield: 86%. ¹H NMR (400 MHz, DMSO-d₆) δ 3.14-3.38 (m, 3H), 3.51-3.74 (m, 4H), 4.60 (dd, J=15.6, 5.1 Hz, 1H), 4.86 (dd, J=15.7, 6.5 Hz, 1H), 7.17-7.36 (m, 4H), 7.58 (d, J=7.1 Hz, 1H), 7.63-7.82 (m, 1H), 8.33 (dd, J=15.0, 7.5 Hz, 2H), 8.63 (d, J=6.6 Hz, 1H), 8.95 (s, 1H), 9.60 (brs, 1H), 9.79 (s, 1H), 9.88 (brs, 1H), 9.60 (s, 1H). HPLC purity: ≥95%. MS-ESI(+) m/z: 332.3 (M+H).

### Example 131: (±)-trans-4-Phenyl-N-[2-(pyridin-3-yloxy)phenyl]pyrrolidine-3-carboxamide dihydrochloride (Compound I-68)

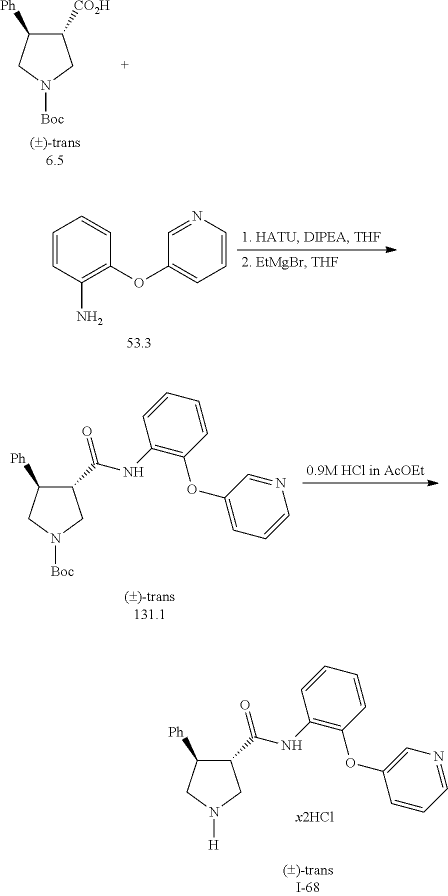 [SMILES: CC(C)(C)OC(=O)N1C[C@@H](C(=O)O)[C@H](c2ccccc2)C1 ; Nc1ccccc1Oc1cccnc1 ; CC(C)(C)OC(=O)N1C[C@@H](C(=O)Nc2ccccc2Oc2cccnc2)[C@H](c2ccccc2)C1 ; O=C(Nc1ccccc1Oc1cccnc1)[C@@H]1CNC[C@H]1c1ccccc1]

### Step 1: (±)-trans tert-Butyl-4-phenyl-N-[2-(pyridin-3-yloxy)phenyl]pyrrolidine-3-carboxamide-1-carboxylate 6737.2)

Intermediate 131.1 was prepared according to the procedure described in Step 1 of Example 64 starting from a solution of intermediate 6.5 (250 mg, 0.86 mmol), HATU (424 mg, 1.12 mmol), DIPEA (0.45 mL, 2.58 mmol), 3.0 M EtMgBr in Et₂O (0.86 mL, 2.58 mmol), and intermediate 53.3 (240 mg, 1.29 mmol) in THF (5 mL+5 mL). Stirring was continued at r.t. for 16 h. After purification by flash chromatography (DCM/MeOH from 100% DCM to 97:3 v/v DCM/MeOH), the intermediate 131.2 (150 mg, 0.33 mmol) was obtained as a colorless oil. Yield 38%.

MS-ESI(+) m/z: 420.6 (M+H).

### Step 2: (±)-trans-4-Phenyl-N-[2-(pyridin-3-yloxy)phenyl]pyrrolidine-3-carboxamide dihydrochloride (Compound I-68)

Compound I-68 was prepared following the procedure described in Step 2 of Example 120 starting from a solution of intermediate 131.1 (150 mg, 0.33 mmol) and a 0.9 M solution of HCl in EtOAc (3.6 mL). The title compound I-68 (134 mg, 0.31 mmol) was obtained as a yellowish solid. Yield: 95%. ¹H NMR (400 MHz, DMSO-d₆) δ 3.19-3.25 (m, 2H), 3.47-3.52 (m, 1H), 3.54-3.73 (m, 4H), 7.02-7.13 (m, 1H), 7.16-7.33 (m, 7H), 7.52 (d, J=8.8 Hz, 1H), 7.62 (dd, J=8.5, 5.1 Hz, 1H), 7.67-7.80 (m, 1H), 8.38 (d, J=2.7 Hz, 1H), 8.46 (d, J=5.0 Hz, 1H), 9.52 (s, 1H), 9.90 (s, 2H).

HPLC purity: ≥95%. MS-ESI(+) m/z: 360.3 (M+H).

### Example 132: (±)-trans-N-(3-Phenoxyphenyl)-4-phenylpyrrolidine-3-carboxamide hydrochloride (Compound I-69)

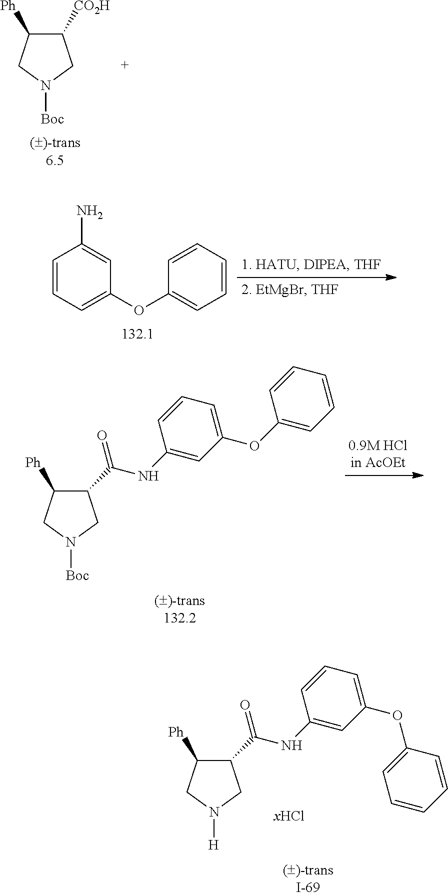 [SMILES: CC(C)(C)OC(=O)N1C[C@@H](C(=O)O)[C@H](c2ccccc2)C1 ; Nc1cccc(Oc2ccccc2)c1 ; CC(C)(C)OC(=O)N1C[C@@H](C(=O)Nc2cccc(Oc3ccccc3)c2)[C@H](c2ccccc2)C1 ; O=C(Nc1cccc(Oc2ccccc2)c1)[C@@H]1CNC[C@H]1c1ccccc1]

### Step 1: (±)-trans tert-butyl-N-(3-Phenoxyphenyl)-4-phenylpyrrolidine-3-carboxamide-1-carboxylate (132.2)

Intermediate 132.2 was prepared according to the procedure described in Step 1 of Example 64 starting from a solution of intermediate 6.5 (200 mg, 0.69 mmol), HATU (341 mg, 0.89 mmol), DIPEA (0.36 mL, 2.1 mmol), 3.0 M EtMgBr in Et₂O (0.7 mL, 2.1 mmol), and intermediate 132.1 (191 mg, 1.03 mmol) in THF (5 mL+5 mL). Stirring was continued at r.t. for 16 h. After purification by flash chromatography (PET/EtOAc from 100% PET to 70:30 v/v PET/EtOAc), the intermediate 132.2 (100 mg, 0.22 mmol) was obtained as a colorless oil. Yield 32%. MS-ESI(+) m/z: 459.5 (M+H).

### Step 2: (±)-trans-N-(3-Phenoxyphenyl)-4-phenylpyrrolidine-3-carboxamide hydrochloride (Compound I-69)

Compound I-69 was prepared following the procedure described in Step 2 of Example 120 starting from a solution of intermediate 132.2 (100 mg, 0.22 mmol) and a 0.9 M solution of HCl in EtOAc (2.5 mL). The title compound I-69 (45 mg, 0.11 mmol) was obtained as a brownish solid. Yield: 52%. ¹H NMR (400 MHz, DMSO-d₆) δ 3.36 (m, 2H), 3.69 (m, 4H), 6.69-6.72 (m, 1H), 6.98-7.01 (m, 2H), 7.12-7.18 (m, 1H), 7.27-7.29 (m, 5H), 7.35-7.41 (m, 5H), 9.39 (brs, 1H), 9.77 (brs, 1H), 10.37 (s, 1H). HPLC purity: ≥95%. MS-ESI(+) m/z: 359.2 (M+H).

### Example 133: (±)-trans-4-Phenyl-N-[4-(pyridin-3-yloxy)phenyl]pyrrolidine-3-carboxamide dihydrochloride (Compound I-70)

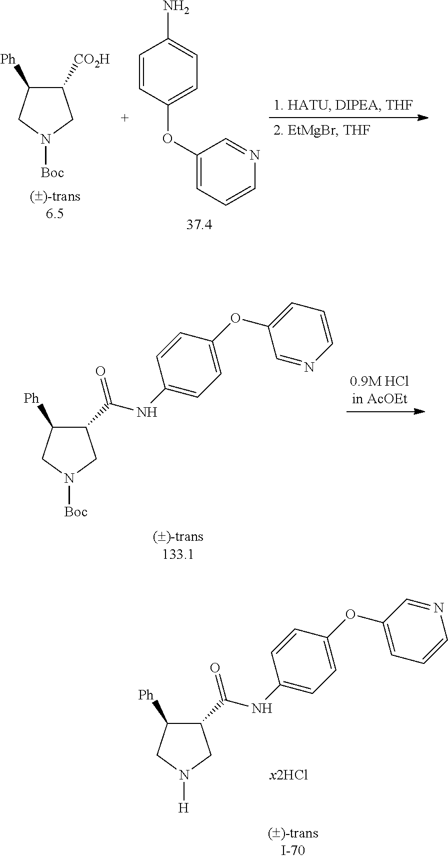 [SMILES: CC(C)(C)OC(=O)N1C[C@@H](C(=O)O)[C@H](c2ccccc2)C1 ; Nc1ccc(Oc2cccnc2)cc1 ; CC(C)(C)OC(=O)N1C[C@@H](C(=O)Nc2ccc(Oc3cccnc3)cc2)[C@H](c2ccccc2)C1 ; O=C(Nc1ccc(Oc2cccnc2)cc1)[C@@H]1CNC[C@H]1c1ccccc1]

### Step 1: (H-trans tert-butyl-N-[4-(Pyridin-3-yloxy)phenyl]pyrrolidine-3-carboxamide-1-carboxylate (133.1)

Intermediate 133.1 was prepared according to the procedure described in Step 1 of Example 64 starting from a solution of intermediate 6.5 (250 mg, 0.86 mmol), HATU (424 mg, 1.12 mmol), DIPEA (0.45 mL, 2.58 mmol), 3.0 M EtMgBr in Et₂O (0.86 mL, 2.58 mmol), and intermediate 37.4 (240 mg, 1.29 mmol) in THF (5 mL+5 mL). Stirring was continued at r.t. for 16 h. After purification by flash chromatography (DCM/MeOH from 100% DCM to 6.5:3.5 v/v DCM/MeOH), the intermediate 133.1 (40 mg, 0.087 mmol) was obtained as a colorless oil. Yield 10%. MS-ESI(+) m/z: 460.5 (M+H).

### Step 2: (±)-trans-4-phenyl-N-[4-(Pyridin-3-yloxy)phenyl]pyrrolidine-3-carboxamide dihydrochloride (Compound I-70)

Compound I-70 was prepared following the procedure described in Step 2 of Example 120 starting from a solution of intermediate 133.1 (30 mg, 0.065 mmol) and a 0.9 M solution of HCl in EtOAc (1 mL). The title compound I-70 (15 mg, 0.03 mmol) was obtained as a brownish solid. Yield: 54%. ¹H NMR (400 MHz, DMSO-d₆) δ 3.26-3.36 (m, 2H), 3.41-3.45 (m, 1H), 3.72-3.78 (m, 4H), 7.10 (d, J=7.2 Hz, 2H), 7.27-7.3 (m, 1H), 7.34-7.41 (m, 4H), 7.62-7.68 (m, 4H), 8.46 (dd, J=4.6 Hz, J=1.5 Hz, 1H), 8.49 (d, J=2.1 Hz, 1H), 9.52 (brs, 1H), 9.91 (brs, 1H), 10.5 (s, 1H).

HPLC purity: ≥95%. MS-ESI(+) m/z: 360.6 (M+H).

### Example 134: (±)-trans 4-Cyclohexyl-N-[3-(pyridin-3-yl)phenyl]pyrrolidine-3-carboxamide dihydrochloride (Compound I-71)

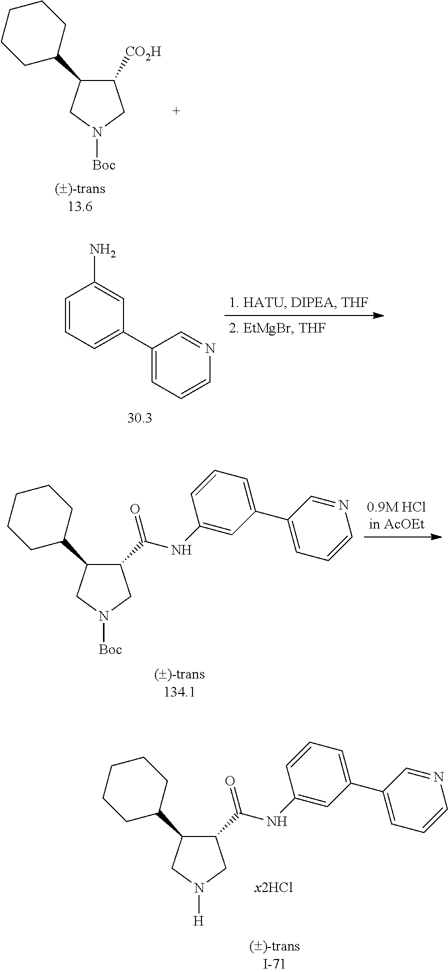 [SMILES: CC(C)(C)OC(=O)N1C[C@@H](C(=O)O)[C@H](C2CCCCC2)C1 ; Nc1cccc(-c2cccnc2)c1 ; CC(C)(C)OC(=O)N1C[C@@H](C(=O)Nc2cccc(-c3cccnc3)c2)[C@H](C2CCCCC2)C1 ; O=C(Nc1cccc(-c2cccnc2)c1)[C@@H]1CNC[C@H]1C1CCCCC1]

### Step 1: tert-Butyl (±)-trans-4-Cyclohexyl-N-[3-(pyridin-3-yl)phenyl]pyrrolidine-3-carboxamide-1-carboxylate (134.1)

Intermediate 134.1 was prepared according to the procedure described in Step 1 of Example 64 starting from intermediate 13.6 (100 mg, 0.34 mmol), HATU (153 mg, 0.40 mmol), DIPEA (0.18 mL, 1.01 mmol), 3.0 M EtMgBr in Et₂O (0.33 mL, 1.01 mmol), and intermediate 30.3 (86 mg, 0.50 mmol) in THF (1.5 mL+1.5 mL). Stirring was continued at r.t. for 16 h. After purification by flash chromatography (DCM/MeOH from 100% DCM to 95:5 v/v DCM/MeOH) the intermediate 134.1 (88 mg, 0.20 mmol) was obtained as a yellowish solid. Yield: 20%. MS-ESI(+) m/z: 450.6 (M+H); MS-ESI(−) m/z: 448.5 (M−H).

### Step 2: (±)-trans 4-Cyclohexyl-N-[3-(pyridin-3-yl)phenyl]pyrrolidine-3-carboxamide dihydrochloride (Compound I-71)

Compound I-71 was prepared following the procedure described in Step 2 of Example 120 starting from a solution of intermediate 134.1 (83 mg, 0.18 mmol) and a 0.9 M solution of HCl in EtOAc (2.1 mL). The title compound I-71 (72 mg, 0.17 mmol) was obtained as a yellowish solid. Yield: 95%. (400 MHz, CDCl₃) δ 0.93-0.96 (m, 2H), 1.36-1.40 (m, 1H), 1.57-1.70 (m, 5H), 2.38-2.43 (m, 1H), 2.62-2.76 (m, 4H), 2.92-2.96 (m, 1H), 3.10-3.15 (m, 2H), 3.39 (brs, 1H), 3.52 (brs, 1H), 7.48-4.54 (m, 2H), 7.73-7.75 (m, 1H), 7.96-7.99 (m, 1H), 8.11 (s, 1H), 8.58 (d, J=7.6 Hz, 1H), 8.82 (d, J=5.4 Hz, 1H), 9.06 (s, 1H), 9.38 (brs, 1H), 9.74 (brs, 1H), 10.84 (s, 1H). HPLC purity: ≥95%. MS-ESI(+) m/z: 350.6 (M+H).

### Example 135: (±)-trans 4-Benzyl-N-[3-(pyridin-3-yl)phenyl]pyrrolidine-3-carboxamide dihydrochloride (Compound I-72)

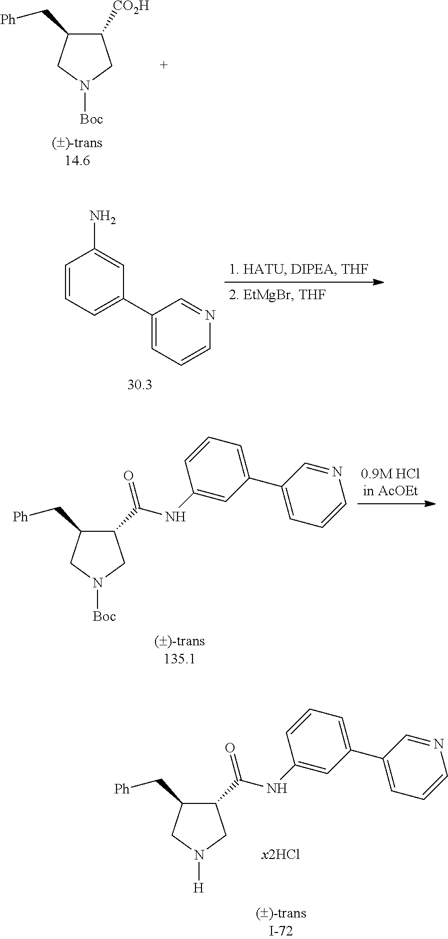 [SMILES: CC(C)(C)OC(=O)N1C[C@@H](Cc2ccccc2)[C@H](C(=O)O)C1 ; Nc1cccc(-c2cccnc2)c1 ; CC(C)(C)OC(=O)N1C[C@@H](Cc2ccccc2)[C@H](C(=O)Nc2cccc(-c3cccnc3)c2)C1 ; O=C(Nc1cccc(-c2cccnc2)c1)[C@@H]1CNC[C@H]1Cc1ccccc1]

### Step 1: tert-Butyl (±)-trans 4-benzyl-N-[3-(pyridin-3-yl)phenyl]pyrrolidine-3-carboxamide-1-carboxylate (135.1)

Intermediate 135.1 was prepared according to the procedure described in Step 1 of Example 64 starting from intermediate 14.6 (100 mg, 0.33 mmol), HATU (150 mg, 0.39 mmol), DIPEA (0.17 mL, 0.98 mmol), 3.0 M EtMgBr in Et₂O (0.32 mL, 0.98 mmol), and intermediate 30.3 (83 mg, 0.49 mmol) in THF (1.5 mL+1.5 mL). Stirring was continued at r.t. for 16 h. After purification by flash chromatography (DCM/MeOH from 100% DCM to 95:5 v/v DCM/MeOH) the intermediate 135.1 (55 mg, 0.12 mmol) was obtained. Yield: 36%. MS-ESI(+) m/z: 458.6 (M+H); MS-ESI(−) m/z: 456.2 (M−H).

### Step 2: (±)-trans 4-Benzyl-N-[3-(pyridin-3-yl)phenyl]pyrrolidine-3-carboxamide dihydrochloride (Compound I-72)

Compound I-72 was prepared following the procedure described in Step 2 of Example 120 starting from a solution of intermediate 135.1 (52 mg, 0.11 mmol) and a 0.9 M solution of HCl in EtOAc (1.3 mL). The title compound I-72 (43 mg, 0.10 mmol) was obtained as a white solid. Yield: 91%. (400 MHz, DMSO-d₆) δ 2.75-2.80 (m, 2H), 2.89-2.95 (m, 2H), 3.14-3.20 (m, 2H), 3.28-3.32 (m, 1H), 3.55-3.60 (m, 1H), 7.12-7.16 (m, 1H), 7.22-7.27 (m, 4H), 7.49-5.52 (m, 2H), 7.67-7.70 (m, 1H), 7.98-8.01 (m, 1H), 8.07 (s, 1H), 8.59-8.61 (m, 1H), 8.83 (d, J=5.3 Hz, 1H), 9.06 (s, 1H), 9.42 (brs, 1H), 1.09 (brs, 1H), 10.81 (s, 1H). HPLC purity: ≥95%. MS-ESI(+) m/z: 358.6.

### Example 136: 5-({[(±)-trans-4-Phenylpyrrolidin-3-yl]oxy}methyl)isoquinoline dihydrochloride (Compound I-73)

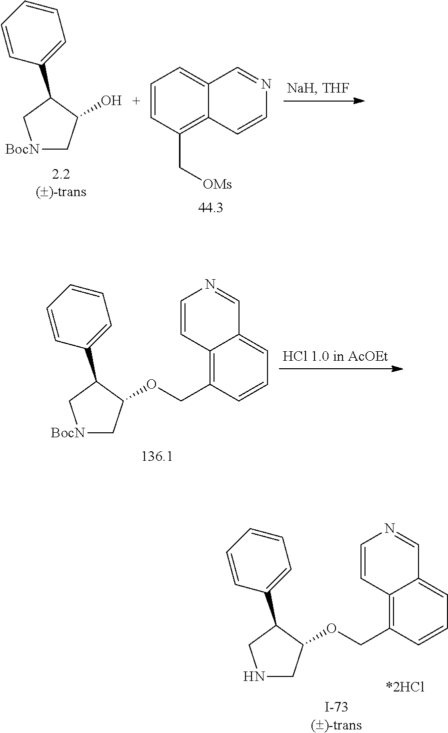

### Step 1: tert-Butyl-({[(±)-trans-4-phenylpyrrolidin-3-yl]oxy}methyl)isoquinoline1-1carboxylate (136.1)

Intermediate 2.2 (106 mg, 0.40 mmol) was added dropwise to a stirred suspension of NaH (60% in mineral oil) (24 mg, 0.60 mmol) in dry THF (10 mL) under N₂ atmosphere and the resulting mixture was reacted at r.t. for 5 min. A solution of intermediate 44.3 (115 mg, 0.48 mmol) in dry THF (10 mL) was then added dropwise and the mixture was stirred at r.t. for 24 h. The mixture was poured into H₂O (30 mL) and extracted with EtOAc (3×10 mL). The combined organic layers were washed with H₂O (20 mL) and brine (20 mL), dried over anhydrous Na₂SO₄, and evaporated to dryness. After purification by flash chromatography (PET/EtOAc, from 85:15 v/v to 3:7 v/v) the desired intermediate 136.1 (11 mg, 0.03 mmol) was obtained. Yield: 7%. MS-ESI(+) m/z: 405.6.

### Step 2: 5-({[(±)-trans-4-Phenylpyrrolidin-3-yl]oxy}methyl)isoquinoline dihydrochloride (Compound I-73)

Compound I-73 was prepared following the procedure described in Step 2 of Example 120 starting from a solution of intermediate 136.1 (11 mg, 0.03 mmol) and a 0.9 M solution of HCl in EtOAc (0.3 mL). The title compound I-73 (7.7 mg, 0.02 mmol) was obtained as a yellowish solid. Yield: 68%. (400 MHz, DMSO-d₆) δ 3.29-3.34 (m, 2H), 3.52-3.56 (m, 1H), 3.64-3.68 (m, 1H), 3.36-3.38 (m, 1H), 5.01 (d, J=12.2 Hz, 1H), 5.04 (d, J=12.2 Hz, 1H), 7.24-7.34 (m, 5H), 7.84-7.87 (m, 1H), 8.02 (d, J=7.0 Hz, 1H), 8.27 (d, J=6.1 Hz, 1H), 8.36 (d, J=8.2 Hz, 1H), 8.59 (d, J=6.5 Hz, 1H), 9.69-9.76 (m, 3H). HPLC purity: ≥95%. MS-ESI(+) m/z: 305.5 (M+H).

### Example 137: (3R,4S)—N-(Naphthalen-1-yl)-4-phenylpyrrolidine-3-carboxamide hydrochloride (Compound I-74)

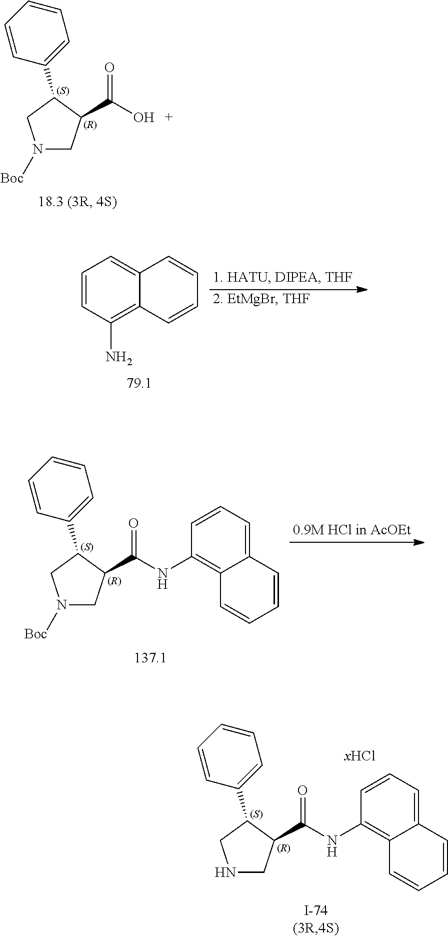

### Step 1: (±)-trans tert-Butyl-N-(naphthalen-1-yl)-4-phenylpyrrolidine-3-carboxamide-1-carboxylate (137.7)

Intermediate 137.1 was prepared according to the procedure described in Step 1 of Example 64 starting from a solution of intermediate 18.3 (250 mg, 0.86 mmol), HATU (420 mg, 1.12 mmol), DIPEA (0.45 mL, 2.58 mmol), 3.0 M EtMgBr in Et₂O (0.86 mL, 2.57 mmol), and intermediate 79.1 (147 mg, 1.03 mmol) in THF (5 mL+5 mL). Stirring was continued at r.t. for 16 h. After purification by flash chromatography (PET/EtOAc from 100% PET to 75:25 v/v PET/EtOAc), the intermediate 137.1 (154 mg, 0.37 mmol) was obtained as a colorless oil. Yield 40%.

### Step 2: (3R,4S)—N-(Naphthalen-1-yl)-4-phenylpyrrolidine-3-carboxamide hydrochloride (Compound I-74)

Compound I-74 was prepared following the procedure described in Step 2 of Example 120 starting from a solution of intermediate 137.1 (154 mg, 0.36 mmol) and a 0.9 M solution of HCl in EtOAc (4 mL). The title compound I-74 (80 mg, 0.23 mmol) was obtained as a white solid. Yield: 63%.

¹H NMR (400 MHz, DMSO-d₆) δ 3.34 (s, 1H), 3.45-3.47 (m, 1H), 3.63-3.76 (m, 3H), 3.81-3.85 (m, 1H), 7.35-7.54 (m, 10H), 7.76 (d, J=8 Hz, 1H), 7.90 (d, J=8 Hz, 1H), 9.65 (brs, 1H), 9.98 (brs, 1H), 10.17 (s, 1H). HPLC purity: ≥95%. MS-ESI(+) m/z: 317.4 (M+H).

### Example 138: (3S,4R)—N-(Naphthalen-1-yl)-4-phenylpyrrolidine-3-carboxamide hydrochloride (Compound I-75)

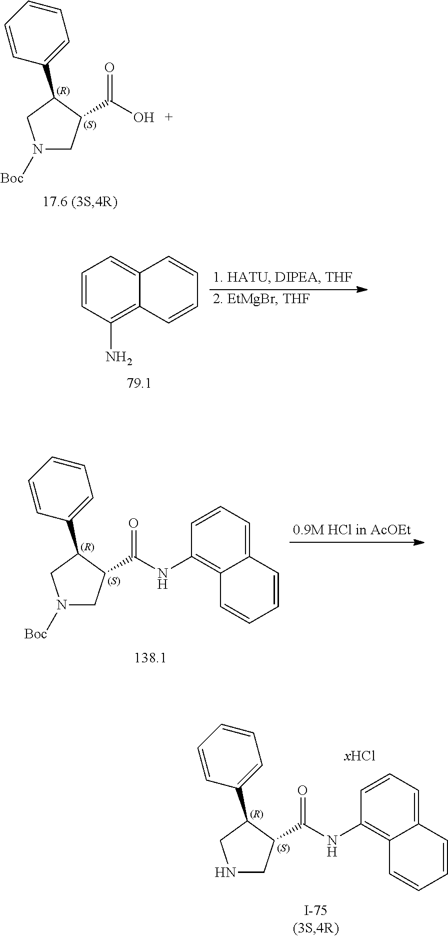

### Step 1: tert-Butyl (3S,4R)—N-(naphthalen-1-yl)-4-phenylpyrrolidine-3-carboxamide-1-carboxylate (138.1)

Intermediate 138.1 was prepared according to the procedure described in Step 1 of Example 64 starting from a solution of intermediate 17.6 (250 mg, 0.86 mmol), HATU (420 mg, 1.11 mmol), DIPEA (0.45 mL, 2.58 mmol), 3.0 M EtMgBr in Et₂O (0.86 mL, 2.57 mmol), and intermediate 79.1 (147 mg, 1.03 mmol) in THF (5 mL+5 mL). Stirring was continued at r.t. for 16 h. After purification by flash chromatography (PET/EtOAc from 100% PET to 75:25 v/v PET/EtOAc), the intermediate 138.1 (90 mg, 0.25 mmol) was obtained as a colorless oil. Yield 25%.

MS-ESI(+) m/z: 417.5 (M+H).

### Step 2: (3S,4R)—N-(Naphthalen-1-yl)-4-phenylpyrrolidine-3-carboxamide hydrochloride (Compound I-75) (Compound I-75)

Compound I-75 was prepared following the procedure described in Step 2 of Example 120 starting from a solution of intermediate 138.1 (154 mg, 0.36 mmol) and a 0.9 M solution of HCl in EtOAc (4 mL). The title compound I-75 (50 mg, 0.14 mmol) was obtained as a white solid. Yield: 74%.

¹H NMR (400 MHz, DMSO-d₆) δ 3.32-3.35 (m, 1H), 3.39-3.45 (m, 1H), 3.60-3.68 (m, 2H), 3.71 (m, 1H), 3.78-3.83 (m, 1H), 7.33-7.52 (m, 10H), 7.74 (d, J=8 Hz, 1H), 8.85 (d, J=8 Hz, 1H), 9.69 (brs, 2H), 10.14 (s, 1H). HPLC purity: ≥95%. MS-ESI(+) m/z: 317.6 (M+H).

### Example 139: (±)-trans-N-(1,3-Benzoxazol-4-yl)-4-phenylpyrrolidine-3-carboxamide dihydrochloride (Compound I-76)

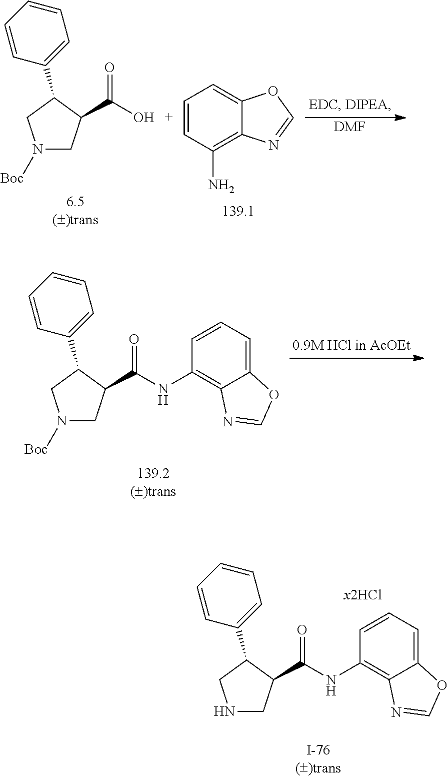

### Step 1: tert-Butyl (±)trans-N-(1,3-benzoxazol-4-yl)-4-phenylpyrrolidine-3-carboxamide-1-carboxylate (139.2)

To a solution of intermediate 6.5 (200 mg, 0.69 mmol) in dry DMF (1 mL) under N₂ atmosphere, DIPEA (0.82 mL, 4.12 mmol), EDC (316 mg, 1.64 mmol), and intermediate 139.1 (138 mg, 1.03 mmol) were sequentially added and the resulting mixture was reacted at r.t. for 3 days. The mixture was poured into 0.5 M aq. citric acid (30 mL) and extracted with CH₂Cl₂ (3×20 mL). The combined organic layers were washed with H₂O (30 mL) and brine (30 mL), dried over anhydrous Na₂SO₄, and evaporated under vacuum. After purification by flash chromatography (DCM/MeOH from 100% DCM to 9:1 v/v DCM/MeOH), the intermediate 139.2 (56 mg, 0.14 mmol) was obtained as a yellowish solid. Yield 20%. MS-ESI(+) m/z: 408.3 (M+H).

### Step 2: (±)trans-N-(1,3-Benzoxazol-4-yl)-4-phenylpyrrolidine-3-carboxamide dihydrochloride (Compound I-76)

Compound I-76 was prepared following the procedure described in Step 2 of Example 120 starting from a solution of intermediate 139.2 (56 mg, 0.14 mmol) and a 0.9 M solution of HCl in EtOAc (1.5 mL). The title compound I-76 (45 mg, 0.12 mmol) was obtained as a white solid. Yield: 86%. (400 MHz, DMSO-d₆) δ 3.26-3.46 (m, 3H), 3.68-3.79 (m, 3H), 6.69-6.79 (m, 1H), 6.92-7.04 (m, 1H), 7.28-7.50 (m, 5H), 7.99-8.04 (m, 1H), 8.86-9.26 (m, 3H). HPLC purity: ≥90%. MS-ESI(+) m/z: 308.3 (M+H).

### Example 140: Isoquinolin-5-yl(3-phenylpyrrolidin-1-yl)methanone (1-77)

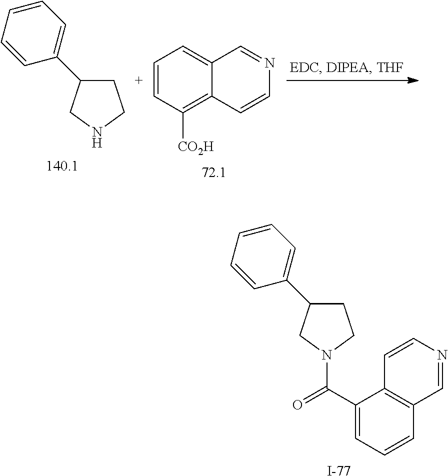

Compound I-77 was prepared starting from intermediate 72.1 (82 mg, 0.48 mmol), EDC (110 mg, 0.57 mmol), and DIPEA (0.25 mL, 1.4 mmol) which were sequentially added to a solution of intermediate 140.1 (70 mg, 0.48 mmol) in THF (5 mL), and the resulting mixture was reacted at r.t. for 24 h. The mixture was poured into H₂O (30 mL) and extracted with EtOAc (3×10 mL). The combined organic layers were washed with H₂O (20 mL), then brine (20 mL), dried over anhydrous Na₂SO₄, and evaporated to dryness. After purification by flash chromatography (DCM/MeOH, from 98:2 v/v to 94:6 v/v) the title compound I-77 (93 mg, 0.31 mmol) was obtained. Yield: 65%. (400 MHz, DMSO-d₆), two rotamers, δ 1.90-1.94 (m, 1H of rotamer 1) 1.98-2.06 (m, 1H of rotamer 2), 2.02-2.08 (m, 1H of rotamer 1), 2.25-2.31 (m, 1H of rotamer 2), 3.12-3.22 (m, 2H of rotamer 1 and 1H of rotamer 2), 3.32-3.54 (m, 2H of rotamer 1 and 2H of rotamer 2) 3.60-3.66 (m, 1H of rotamer 2), 3.82-3.86 (m, 1H of rotamer 1), 4.06-4.11 (m, 1H of rotamer 2), 7.11-7.22 (m, 3H+3H of both rotamers), 7.31 (m, 2H+2H of both rotamers), 7.64-7.71 (m, 2H+2H of both rotamers), 7.77-7.79 (m, 1H+1H of both rotamers), 8.12-8.17 (m, 1H+1H of both rotamers), 8.48-8.52 (m, 1H+1H of both rotamers), 9.33 (d, J=11.1 Hz, 1H+1H of both rotamers). HPLC purity: ≥95%. MS-ESI(+) m/z: 303.4 (M+H).

### Example 141: N-(Isoquinolin-5-yl)-3-phenylpyrrolidine-1-carbothioamide (Compound I-78)

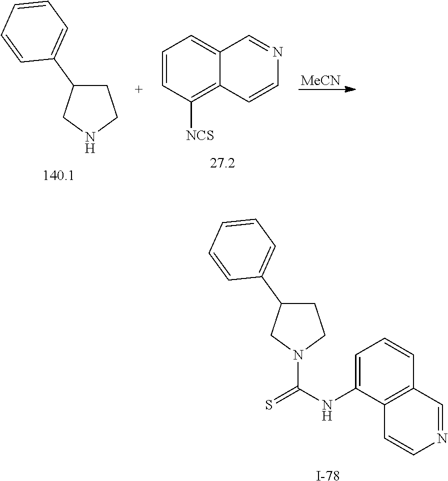

Compound I-78 was prepared following the procedure described in Example 69 starting from a solution of intermediate 140.1 (70 mg, 0.48 mmol) and intermediate 27.2 (97 mg, 0.52 mmol) in MeCN (1.5 mL). Stirring was continued for 2 h. The title compound I-78 (82 mg, 0.24 mmol) was obtained after recrystallization from MeCN as a yellowish solid. Yield: 51%. (400 MHz, DMSO-d₆) δ 2.02-2.39 (m, 2H), 3.60-3.77 (m, 3H), 4.01 (brs, 1H), 4.23-4.27 (m, 1H), 7.25-7.28 (m, 1H), 7.37-7.41 (m, 4H), 7.63-7.70 (m, 2H), 7.77 (d, J=5.8 Hz, 1H), 8.03 (d, J=8.0 Hz, 1H), 8.49 (d, J=5.9 Hz, 1H), 9.23 (s, 1H), 9.32 (s, 1H). HPLC purity: ≥95%. MS-ESI(+) m/z: 334.4 (M+H).

### Example 142: N-(Isoquinolin-5-yl)-3-phenylpyrrolidine-1-carboxamide (Compound I-79)

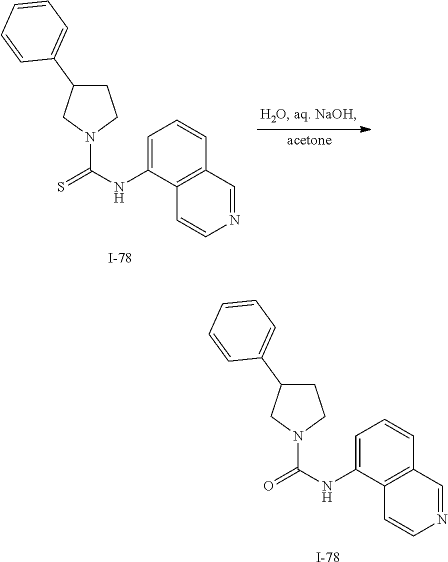

Compound I-79 was prepared following the procedure described in Step 1 of Example 67 starting from compound I-78 (229 mg, 0.69 mmol), 30% aq. H₂O₂ (2.1 mL, 20.61 mmol), and 10% aq. NaOH (8.24 mL, 20.61 mmol) in AcMe (9 mL). Stirring was continued for 24 h. After purification by flash chromatography (DCM/MeOH from 100% DCM to 95:5 v/v DCM/MeOH), the title compound I-79 (146 mg, 0.46 mmol) was obtained as a white solid. Yield: 67%. (400 MHz, DMSO-d₆) δ 2.05-2.12 (m, 1H), 2.30-2.34 (m, 1H), 3.42-3.56 (m, 3H), 3.73-3.77 (m, 1H), 3.98-4.01 (m, 1H), 7.25-7.28 (m, 1H), 7.34-7.38 (m, 4H), 7.64 (t, J=7.8 Hz, 1H), 7.81 (d, J=7.4 Hz, 1H), 7.85-7.90 (m, 2H), 8.37 (s, 1H), 8.49 (d, J=5.9 Hz, 1H), 9.29 (s, 1H). HPLC purity: ≥95%. MS-ESI(+) m/z: 318.5 (M+H).

### Example 143: 5-[(3-Phenylpyrrolidin-1-yl)sulfonyl]isoquinoline (Compound I-80)

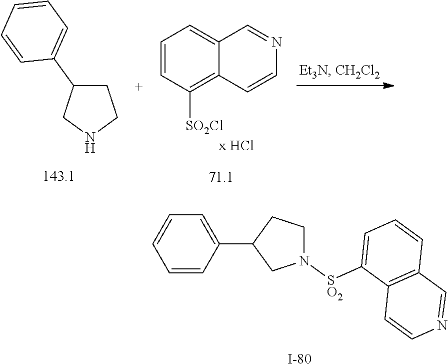

Compound I-80 was prepared following the procedure described in Step 1 of Example 71 starting from intermediate 143.1 (70 mg, 0.48 mmol), intermediate 71.1 (138 mg, 0.52 mmol), and Et₃N (0.17 mL, 1.19 mmol) in DCM (5 mL). Stirring was continued for 24 h. After purification by flash chromatography (DCM/MeOH from 100% DCM to 94:6 v/v DCM/MeOH), the title compound I-80 (138 mg, 0.41 mmol) was obtained as a white solid. Yield: 85%. (400 MHz, DMSO-d₆) δ 1.87-1.94 (m, 1H), 2.20-2.24 (m, 1H), 3.19 (t, J=9.1 Hz, 1H), 3.33-3.44 (m, 1H), 3.52-3.57 (m, 1H), 3.78 (dd, J1=12.2 Hz, J2=3.3 Hz, 1H), 7.13-7.25 (m, 4H), 7.85-7.90 (m, 1H), 8.40 (dd, J1=7.4 Hz, J2=1.1 Hz, 1H), 8.48-8.52 (m, 2H), 8.70 (d, J=6.1 Hz, 1H), 9.50 (s, 1H). HPLC purity: ≥95%. MS-ESI(+) m/z: 339.4 (M+H).

### Example 144: (3R,4S)-4-(4-Fluorophenyl)-N-(isoquinolin-5-yl)pyrrolidine-3-carboxamide dihydrochloride (Compound I-81)

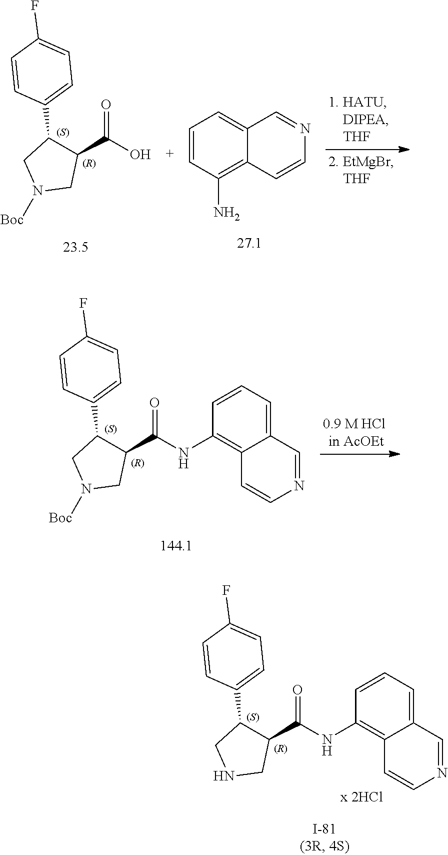

### Step 1: tert-Butyl (3R,4S)-4-(4-fluorophenyl)-N-(isoquinolin-5-yl)pyrrolidine-3-carboxamide-1-carboxylate (144.1)

Intermediate 144.1 was prepared according to the procedure described in Step 1 of Example 64 starting from a solution of intermediate 23.5 (250 mg, 0.81 mmol), HATU (399 mg, 1.05 mmol), DIPEA (0.42 mL, 2.43 mmol), 3.0 M EtMgBr in Et₂O (0.81 mL, 2.43 mmol), and intermediate 27.1 (140 mg, 0.97 mmol) in THF (5 mL+5 mL). Stirring was continued at r.t. for 16 h. After purification by flash chromatography on NH-based silica gel, eluting with (PET/EtOAc from 100% PET to 70:30 v/v PET/EtOAc), the intermediate 144.1 (160 mg, 0.45 mmol) was obtained as a brownish oil. Yield 56%. MS-ESI(+) m/z: 436.6 (M+H).

### Step 2: (3R,4S)-4-(4-Fluorophenyl)-N-(isoquinolin-5-yl)pyrrolidine-3-carboxamide dihydrochloride (Compound I-81)

Compound I-81 was prepared following the procedure described in Step 2 of Example 120 starting from a solution of intermediate 144.1 (150 mg, 0.34 mmol) and a 0.9 M solution of HCl in EtOAc (4.3 mL). The title compound I-81 (100 mg, 0.24 mmol) was obtained as a white solid. Yield: 72%.

Yield: 72%. ¹H NMR (400 MHz, DMSO-d₆) δ 3.31 (m, 1H), 3.42 (m, 1H), 3.73 (m, 3H), 3.84 (m, 1H), 7.22 (t, J=8.7 Hz, 2H), 7.52 (dd, J=8.2, 5.6 Hz, 2H), 7.93 (t, J=1.9 Hz, 1H), 8.08 (d, J=6.3 Hz, 2H), 8.17 (d, J=7.5 Hz, 1H), 8.30 (d, J=8.2 Hz, 1H), 8.59 (d, J=6.6 Hz, 1H), 9.82 (m, 2H), 10.16 (brs, 1H), 10.87 (s, 1H). HPLC purity: ≥95%. MS-ESI(+) m/z: 336.7 (M+H).

### Example 145: (3R,4S)-4-(4-Trifluorophenyl)-N-(isoquinolin-5-yl)pyrrolidine-3-carboxamide dihydrochloride (Compound I-82)

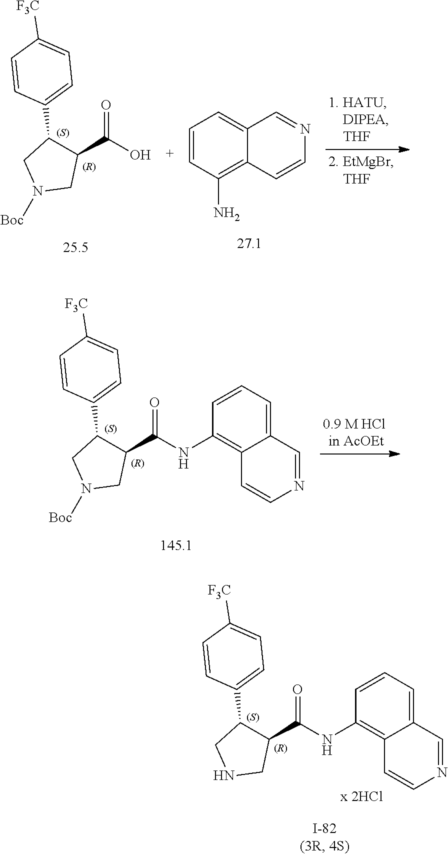

### Step 1: tert-Butyl (3R,4S)-4-(4-trifluorophenyl)-N-(isoquinolin-5-yl)pyrrolidine-3-carboxamide-1-carboxylate (145.1)

Intermediate 145.1 was prepared according to the procedure described in Step 1 of Example 64 starting from intermediate 25.5 (250 mg, 0.70 mmol), HATU (317 mg, 0.83 mmol), DIPEA (0.37 mL, 2.09 mmol), 3.0 M EtMgBr in Et₂O (0.70 mL, 2.09 mmol), and intermediate 27.1 (301 mg, 2.09 mmol) in THF (3.5 mL+3.5 mL). Stirring was continued at r.t. for 16 h. After purification by flash chromatography (DCM/MeOH from 100% DCM to 95:5 v/v DCM/MeOH) the intermediate 145.1 (63 mg, 0.13 mmol) was obtained as a white solid. Yield 19%. MS-ESI(+) m/z: 486.5 (M+H); MS-ESI(−) m/z: 484.3 (M−H).

### Step 2: (3R,4S)-4-(4-Trifluorophenyl)-N-(isoquinolin-5-yl)pyrrolidine-3-carboxamide dihydrochloride (Compound I-82)

Compound I-82 was prepared following the procedure described in Step 1 of Example 120 starting from a solution of intermediate 145.1 (63 mg, 0.13 mmol) and a 0.9 M solution of HCl in EtOAc (1.4 mL). The title compound I-82 (51 mg, 0.11 mmol) was obtained as a white solid. Yield: 85%. (400 MHz, DMSO-d₆) δ 3.40-3.46 (m, 2H), 3.79-3.87 (m, 4H), 7.74 (m, 4H), 7.91 (t, J=8.0 Hz, 1H), 8.05 (d, J=6.5 Hz, 1H), 8.17 (d, J=7.6 Hz, 1H), 8.28 (d, J=8.3 Hz, 1H), 8.54 (d, J=6.6 Hz, 1H), 9.78 (brs, 2H), 10.17 (brs, 1H), 10.84 (s, 1H). HPLC purity: ≥95%. MS-ESI(+) m/z: 386.6 (M+H).

### Example 146: (3R,4S)—N-(1-Chloroisoquinolin-5-yl)-4-phenylpyrrolidine-3-carboxamide dihydrochloride (Compound I-83)

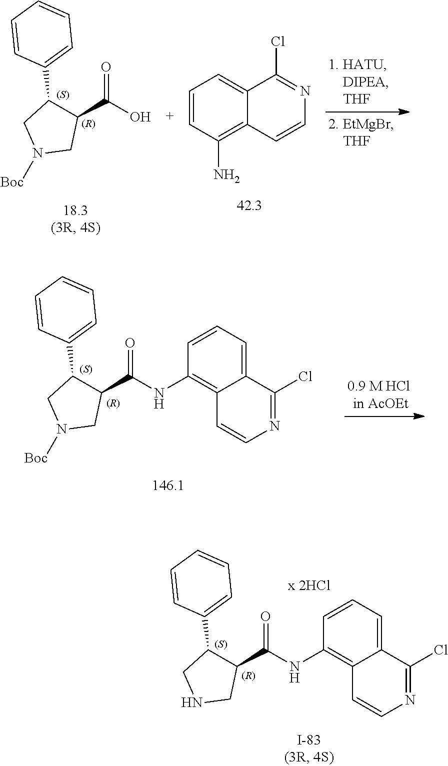

### Step 1: tert-Butyl (3R,4S)—N-(1-chloroisoquinolin-5-yl)-4-phenylpyrrolidine-3-carboxamide-1-carboxylate (146.1)

Intermediate 146.1 was prepared according to the procedure described in Step 1 of Example 64 starting from intermediate 18.3 (100 mg, 0.34 mmol), HATU (157 mg, 0.41 mmol), DIPEA (0.18 mL, 1.03 mmol), 3.0 M EtMgBr in Et₂O (0.34 mL, 1.03 mmol), and intermediate 42.3 (184 mg, 1.03 mmol) in THF (2.0 mL+2.0 mL). Stirring was continued at r.t. for 16 h. After purification by flash chromatography (DCM/MeOH from 100% DCM to 9:1 v/v DCM/MeOH) the intermediate 146.1 (57 mg, 0.13 mmol) was obtained as a yellowish solid. Yield 38%. MS-ESI(+) m/z: 452.6 (M+H); MS-ESI(−) m/z: 450.5 (M−H).

### Step 2: (3R,4S)—N-(1-Chloroisoquinolin-5-yl)-4-phenylpyrrolidine-3-carboxamide dihydrochloride (Compound I-83)

Compound I-83 was prepared following the procedure described in Step 2 of Example 120 starting from a solution of intermediate 146.1 (57 mg, 0.13 mmol) and a 0.9 M solution of HCl in EtOAc (1.4 mL). The title compound I-83 (46 mg, 0.11 mmol) was obtained as a yellowish solid. Yield: 85%. (400 MHz, DMSO-d₆) δ 3.31-3.44 (m, 2H), 3.67-3.85 (m, 4H), 7.33-7.46 (m, 7H), 7.76 (t, J=8.1 Hz, 1H), 7.92 (d, J=7.5 Hz), 8.10 (d, J=8.4 Hz, 1H), 8.16 (d, J=5.9 Hz, 1H), 9.65 (brs, 1H), 10.02 (brs, 1H), 10.44 (s, 1H). HPLC purity: ≥95%. MS-ESI(+) m/z: 352.6 (M+H).

### Example 147: (3R,4S)—N-(2-Methyl-1-oxo-1,2-dihydroisoquinolin-5-yl)-4-phenylpyrrolidine-3-carboxamide hydrochloride (Compound I-84)

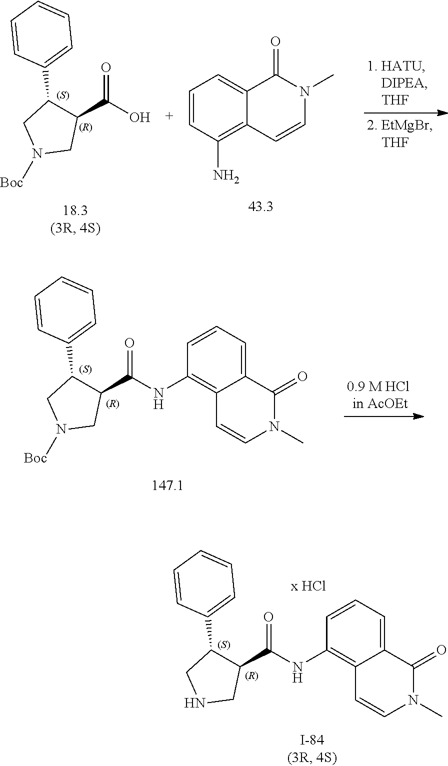

### Step 1: tert-Butyl (3R,4S)—N-(2-methyl-1-oxo-1,2-dihydroisoquinolin-5-yl)-4-phenylpyrrolidine-3-carboxamide-1-carboxylate (147.1)

Intermediate 147.1 was prepared according to the procedure described in Step 1 of Example 64 starting from a solution of intermediate 18.3 (200 mg, 0.69 mmol), HATU (341 mg, 0.9 mmol), DIPEA (0.36 mL, 2.07 mmol), 3.0 M EtMgBr in Et₂O (0.69 mL, 2.07 mmol), and intermediate 43.3 (143 mg, 0.82 mmol) in THF (5 mL+5 mL). Stirring was continued at r.t. for 16 h. After purification by flash chromatography (PET/EtOAc from 100% PET to 70:30 v/v PET/EtOAc), the intermediate 147.1 (30 mg, 0.067 mmol) was obtained as a brownish solid. Yield 10%. MS-ESI(+) m/z: 448.5 (M+H).

### Step 2: (3R,4S)—N-(2-Methyl-1-oxo-1,2-dihydroisoquinolin-5-yl)-4-phenylpyrrolidine-3-carboxamide hydrochloride (Compound I-84)

Compound I-84 was prepared following the procedure described in Step 2 of Example 120 starting from a solution of intermediate 147.1 (20 mg, 0.044 mmol) and a 0.9 M solution of HCl in EtOAc (0.5 mL). The title compound I-84 (15 mg, 0.039 mmol) was obtained as a white solid. Yield: 88%.

¹H NMR (400 MHz, DMSO-d₆) δ 3.29-3.33 (m, 1H), 3.37-3.42 (m, 1H), 3.46 (s, 3H), 3.35-3.66 (m, 2H), 3.68-3.80 (m, 2H), 6.06 (d, J=7.6 Hz, 1H), 7.32-7.35 (m, 2H), 7.38-7.44 (m, 5H), 7.67 (d, J=7.7 Hz, 1H), 8.04 (d, J=8.1 Hz, 1H), 9.58-9.94 (m, 2H), 10.04 (s, 1H). HPLC purity: ≥95%. MS-ESI(+) m/z: 348.6 (M+H).

### Example 148: (±)-trans-N-(3-Benzylphenyl)-4-phenylpyrrolidine-3-carboxamide hydrochloride (Compound I-85)

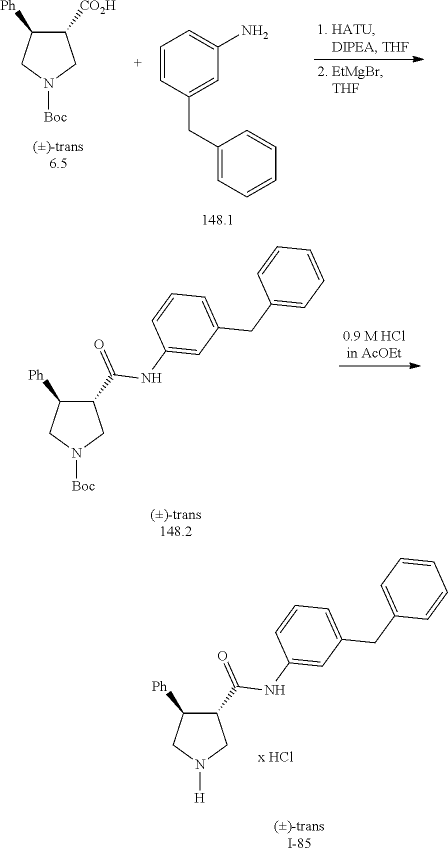 [SMILES: CC(C)(C)OC(=O)N1C[C@@H](C(=O)O)[C@H](c2ccccc2)C1 ; Nc1cccc(Cc2ccccc2)c1 ; CC(C)(C)OC(=O)N1C[C@@H](C(=O)Nc2cccc(Cc3ccccc3)c2)[C@H](c2ccccc2)C1 ; O=C(Nc1cccc(Cc2ccccc2)c1)[C@@H]1CNC[C@H]1c1ccccc1]

### Step 1: (H-trans tert-Butyl-N-(3-Benzylphenyl)-4-phenylpyrrolidine-3-carboxamide-1-carboxylate (148.2)

Intermediate 148.2 was prepared according to the procedure described in Step 1 of Example 64 starting from a solution of intermediate 6.5 (200 mg, 0.68 mmol), HATU (339 mg, 0.9 mmol), DIPEA (0.35 mL, 2.04 mmol), 3.0 M EtMgBr in Et₂O (0.68 mL, 2.04 mmol), and intermediate 148.1 (150 mg, 0.82 mmol) in THF (5 mL+5 mL). Stirring was continued at r.t. for 16 h. After purification by flash chromatography (PET/EtOAc from 100% PET to 80:20 v/v PET/EtOAc), the intermediate 148.2 (110 mg, 0.24 mmol) was obtained as a yellowish oil. Yield 35%. MS-ESI(+) m/z: 457.6 (M+H).

### Step 2: (±)-trans-N-(3-Benzylphenyl)-4-phenylpyrrolidine-3-carboxamide hydrochloride (Compound I-85)

Compound I-85 was prepared following the procedure described in Step 2 of Example 120 starting from a solution of intermediate 148.2 (103 mg, 0.22 mmol) and a 0.9 M solution of HCl in EtOAc (2.5 mL). The title compound I-85 (70 mg, 0.18 mmol) was obtained as a white solid. Yield: 81%. ¹H NMR (400 MHz, DMSO-d₆) δ 3.24 (m, 2H), 3.35-3.41 (m, 2H), 3.68 (m, 3H), 3.86 (s, 2H), 6.91 (d, J=7.6 Hz, 1H), 7.15-7.19 (m, 3H), 7.24-7.28 (m, 2H), 7.30-7.40 (m, 6H), 9.46 (brs, 1H), 9.86 (brs, 1H), 10.21 (s, 1H). HPLC purity: ≥95%. MS-ESI(+) m/z: 357.7 (M+H).

### Example 149: (±)-trans-N-[3-(Tetrahydro-2H-pyran-4-yloxy)phenyl]-4-phenylpyrrolidine-3-carboxamide hydrochloride (Compound I-86)

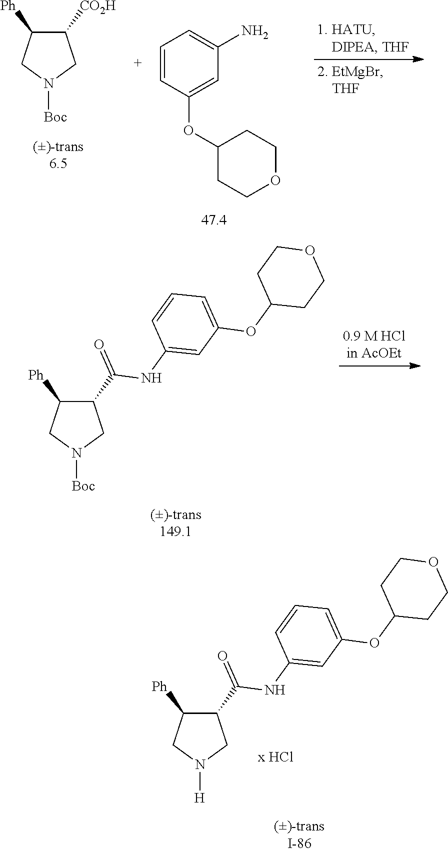 [SMILES: CC(C)(C)OC(=O)N1C[C@@H](C(=O)O)[C@H](c2ccccc2)C1 ; Nc1cccc(OC2CCOCC2)c1 ; CC(C)(C)OC(=O)N1C[C@@H](C(=O)Nc2cccc(OC3CCOCC3)c2)[C@H](c2ccccc2)C1 ; O=C(Nc1cccc(OC2CCOCC2)c1)[C@@H]1CNC[C@H]1c1ccccc1]

### Step 1: (±)-trans tert-Butyl-4-phenyl-N-[3-(tetrahydro-2H-pyran-4-yloxy)phenyl]pyrrolidine-3-carboxamide-1-carboxylate (122.2)

Intermediate 149.1 was prepared according to the procedure described in Step 1 of Example 64 starting from a solution of intermediate 6.5 (200 mg, 0.68 mmol), HATU (339 mg, 0.9 mmol), DIPEA (0.35 mL, 2.04 mmol), 3.0 M EtMgBr in Et₂O (0.68 mL, 2.04 mmol), and intermediate 47.4 (170 mg, 0.88 mmol) in THF (5 mL+5 mL). Stirring was continued at r.t. for 16 h. After purification by flash chromatography (PET/EtOAc from 100% PET to 50:50 v/v PET/EtOAc), the intermediate 149.1 (220 mg, 0.47 mmol) was obtained as a yellowish oil. Yield 66%. MS-ESI(+) m/z: 467.6 (M+H).

### Step 2: (±)-trans-N-[3-(Tetrahydro-2H-pyran-4-yloxy)phenyl]-4-phenylpyrrolidine-3-carboxamide hydrochloride (Compound I-86)

Compound I-86 was prepared following the procedure described in Step 2 of Example 120 starting from a solution of intermediate 149.1 (140 mg, 0.3 mmol) and a 0.9 M solution of HCl in EtOAc (3.75 mL). The title compound I-86 (80 mg, 0.19 mmol) was obtained as a white solid. Yield: 66%. NMR %. ¹H NMR (400 MHz, DMSO-d₆) δ 1.50-1.62 (m, 2H), 1.89-1.97 (m, 2H), 3.25-3.30 (m, 2H), 3.36-3.46 (m, 3H), 3.69-3.73 (m, 3H), 3.79-3.82 (m, 2H), 4.42-4.46 m, 1H), 6.65 (dd, J=8.2, 1.3 Hz, 1H), 7.05 (d, J=8.2 Hz, 1H), 7.14 (t, J=8.1 Hz, 1H), 7.29-7.23 (m, 2H), 7.32-7.38 m, 4H), 9.48 (brs, 1H), 9.87 (brs, 1H), 10.30 (s, 1H). HPLC purity: ≥95%. MS-ESI(+) m/z: 367.7 (M+H).

### Example 150: (±)-trans-N-(4-Cyclohexylphenyl)-4-phenylpyrrolidine-3-carboxamide hydrochloride (Compound I-87)

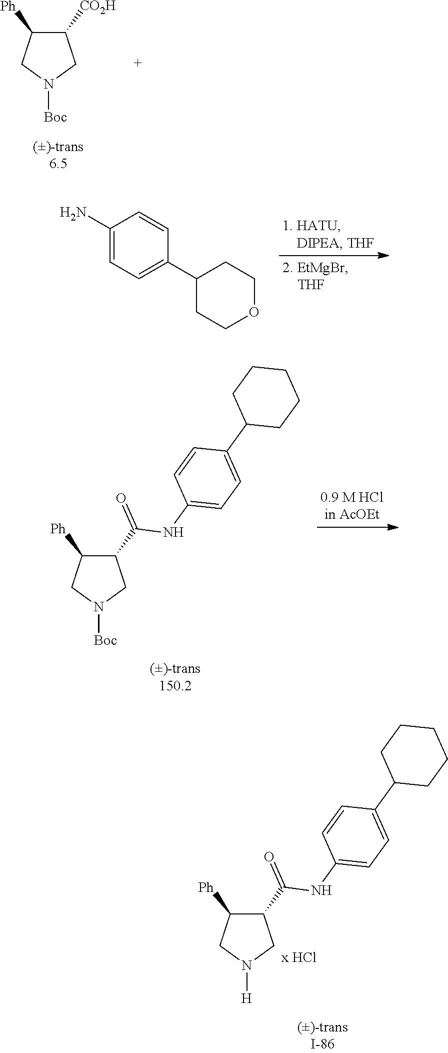

### Step 1: (±)-trans tert-Butyl N-(4-cyclohexylphenyl)-4-methylpyrrolidine-3-carboxamide-1-carboxylate (150.2)

Intermediate 150.2 was prepared according to the procedure described in Step 1 of Example 64 starting from a solution of intermediate 6.5 (200 mg, 0.68 mmol), HATU (339 mg, 0.9 mmol), DIPEA (0.35 mL, 2.04 mmol), 3.0 M EtMgBr in Et₂O (0.68 mL, 2.04 mmol), and intermediate 150.1 (143 mg, 0.81 mmol) in THF (5 mL+5 mL). Stirring was continued at r.t. for 16 h. After purification by flash chromatography (PET/EtOAc from 100% PET to 70:30 v/v PET/EtOAc), the intermediate 150.2 (200 mg, 0.45 mmol) was obtained as a white solid. Yield 66%. MS-ESI(+) m/z: 449.5 (M+H).

### Step 2: (±)-trans-N-(4-Cyclohexylphenyl)-4-phenylpyrrolidine-3-carboxamide hydrochloride (Compound I-87)

Compound I-87 was prepared following the procedure described in Step 2 of Example 120 starting from a solution of intermediate 150.2 (125 mg, 0.28 mmol) and a 0.9 M solution of HCl in EtOAc (3.4 mL). The title compound I-87 (65 mg, 0.17 mmol) was obtained as a white solid. Yield: 60%. Yield: 60%. ¹H NMR (400 MHz, DMSO-d₆) δ 1.14-1.21 (m, 1H), 1.26-1.36 (m, 4H), 1.64-1.74 (m, 5H), 2.39 (m, 1H), 3.25 (m, 2H), 3.36-3.43 (m, 2H), 3.68-3.72 (m, 3H), 7.08 (d, J=8.5 Hz, 2H), 7.18-7.23 (m, 1H), 7.27-7.38 (m, 4H), 7.42 (d, J=8.5 Hz, 2H), 9.67 (brs, 1H), 9.98 (brs, 1H), 10.23 (s, 1H). HPLC purity: ≥95%. MS-ESI(+) m/z: 349.7 (M+H).

### Example 151: (3R,4R)—N-(Isoquinolin-5-yl)-4-phenylpyrrolidine-3-carboxamide dihydrochloride (Compound I-88)

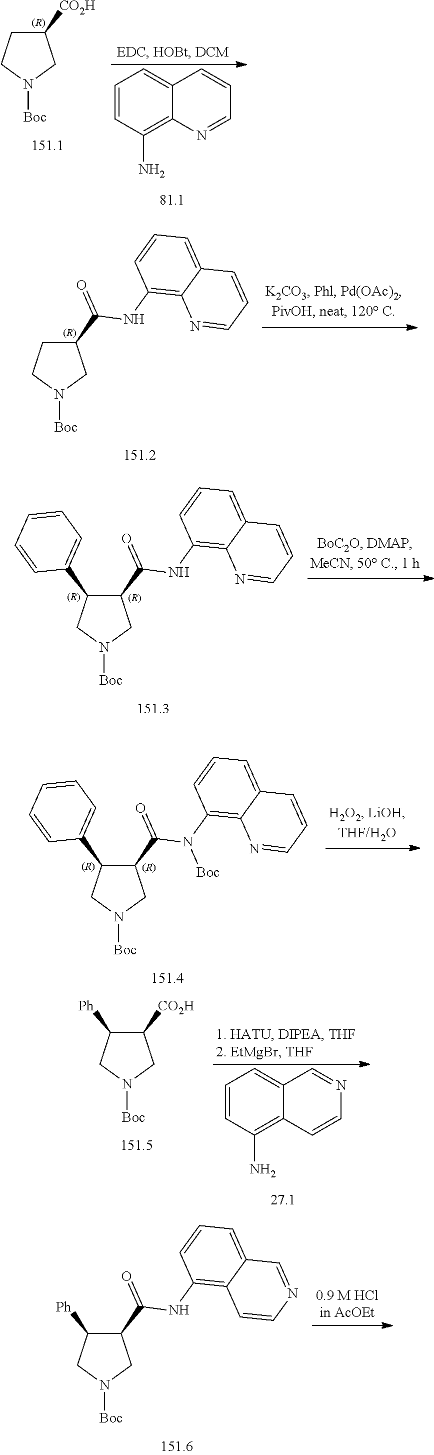

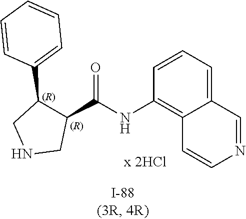

### Step 1: tert-butyl (3R)-3-(Quinolin-8-ylcarbamoyl)pyrrolidine-1-carboxylate (151.2)

HOBt (489 mg, 3.62 mmol) was added to a solution of intermediate 151.1 (650 mg, 3.01 mmol) and intermediate 81.1 (522 mg, 3.62 mmol) in dry DCM (15 mL) and stirring was continued at r.t. for 5 min. EDC (694 mg, 3.62 mmol) was then added to the mixture and stirring was continued at r.t. additional 72 h. The crude was poured into water, diluted with DCM (50 mL), and washed with water (50 mL), 1 M solution of HCl (30 mL), NaHCO₃ (ss) (50 mL), and brine (60 mL). Purification by flash chromatography (PET/EtOAc from 100% PET to 60:40 v/v PET/EtOAc) the intermediate 151.2 (970 mg, 2.85 mmol) was obtained as a yellowish oil. Yield 95%. MS-ESI(+) m/z: 342.2 (M+H).

### Step 2: tert-Butyl (3R,4R)-4-Phenyl-N-(quinolin-8-yl)pyrrolidine-3-carboxamide-1-carboxylate (151.3)

K₂CO₃ (40 mg, 0.29 mmol) was flamed in a dry vial, the vial was then charged under argon with Pd(OAc)₂ (3.2 mg, 0.014 mmol), PivOH (30 mg, 0.29 mmol), and intermediate 151.2 (100 mg, 0.29 mmol). The reaction vessel was sealed and purged with argon (3 times). Iodobenzene (0.16 mL, 0.87 mmol) was added and the mixture was sonicated 5 min under argon. The reaction tube was placed in a preheated bath and stirred at 120° C. 48 h. The reaction was allowed to cool to r.t., then diluted with EtOAc (10 mL) and filtered through a short pad of celite. Purification by flash chromatography (DCM/MeCN from 100% DCM to 10:90 v/v DCM/MeCN) the intermediate 151.3 (55 mg, 0.13 mmol) was obtained as a colorless oil. Yield 45%. MS-ESI(+) m/z: 416.3 (M+H).

### Step 3: (3R,4R)-1-Pyrrolidinecarboxylic acid, 3-[[[(1,1-dimethylethoxy)carbonyl]-8-quinolinylamino]carbonyl]-4-(4-phenyl)-,1,1-dimethylethyl ester (151.4)

A flame dried reaction tube was charged with a solution of intermediate 151.3 (525 mg, 1.65 mmol), Boc2O (1.89 mL, 8.3 mmol), and DMAP (427 mg, 3.3 mmol) in MeCN (3.3 mL). The reaction was heated at 50° C. for 2 h. Then the reaction was allowed to cool to r.t., gently opened and quenched with NH4⁺Cl⁻. The mixture was diluted with DCM (10 mL) and water (15 mL).

The crude was diluted with water and was extracted with DCM (3×30 mL). The organic phase were collected together, washed with brine, and dried over anhydrous Na₂SO₄. Purification (PET/Acetone from 100% PET to 80:20 v/v PET/Acetone) the intermediate 151.4 (638 mg, 1.23 mmol) was obtained as a white solid. Yield 75%. MS-ESI(+) m/z: 518.5 (M+H).

### Step 4: (3R,4R)-1-(tert-Butoxycarbonyl)-4-phenylpyrrolidine-3-carboxylic acid (151.5)

A solution of 30% aq. H₂O₂ (0.32 mL, 3.19 mmol) in THF (3.3 mL) was added under argon at 0° C. to a solution of LiOH (51 mg, 2.12 mmol) in water (3.3 mL). This mixture was added dropwise under argon to a solution of intermediate 151.4 (550 mg, 1.06 mmol) in THF (4.4 mL). The vial was sealed and stirring was continued at r.t. 2 h. The reaction was quenched by addition of Na₂S₂O₃ (ss) and the water was extracted with EtOAc (3×20 mL). The pH was brought to pH=2 by the addition of 1M HCl solution and the aqueous phase was extracted with EtOAc (3×30 mL). The organic phases were collected together, washed with brine, and dried over anhydrous Na₂S04 to afford the intermediate 151.5 (236 mg, 0.81 mmol) as a white solid. Yield 76%. MS-ESI(−) m/z: 290.1 (M−H).

### Step 5: tert-Butyl (3R,4R)—N-(isoquinolin-5-yl)-4-phenylpyrrolidine-3-carboxamide-1-carboxylate (151.6)

Intermediate 151.6 was prepared according to the procedure described in Step 1 of Example 64 starting from a solution of intermediate 151.5 (215 mg, 0.73 mmol), HATU (365 mg, 0.96 mmol), DIPEA (0.39 mL, 2.21 mmol), 3.0 M EtMgBr in Et₂O (0.74 mL, 2.21 mmol), and intermediate 27.1 (159.4 mg, 1.10 mmol) in THF (5 mL+5 mL). Stirring was continued at r.t. for 16 h. After purification by flash chromatography (DCM/MeOH from 100% DCM to 96.5:3.5 v/v DCM/MeOH), the intermediate 151.6 (100 mg, 0.24 mmol) was obtained as a colorless oil. Yield 33%. MS-ESI(+) m/z: 418.3 (M+H); MS-ESI(−) m/z: 416.3 (M−H).

### Step 5: (3R,4R)—N-(Isoquinolin-5-yl)-4-phenylpyrrolidine-3-carboxamide dihydrochloride (Compound I-88)

Compound I-88 was prepared following the procedure described in Step 3 of Example 96 starting from a solution of intermediate 151.6 (40 mg, 0.095 mmol) and a 0.9 M solution of HCl in EtOAc (1.36 mL). The title compound I-88 (20 mg, 0.05 mmol) was obtained as a white solid. Yield: 54%. ¹H NMR (400 MHz, DMSO-d₆) δ 3.57-3.68 (m, 4H), 4.03-4.09 (m, 2H), 7.22 (d, J=7.2 Hz, 1H), 7.29 (t, J=7.4 Hz, 2H), 7.39 (d, J=7.2 Hz, 3H), 7.73 (d, J=7.7 Hz, 2H), 7.84 (t, J=1.9 Hz, 1H), 8.24 (d, J=8.2 Hz, 1H), 8.51 (d, J=6.7 Hz, 1H), 9.66 (brs, 1H), 9.77 (s, 1H), 10.07 (brs, 1H), 10.76 (s, 1H). HPLC purity: ≥95%. MS-ESI(+) m/z: 318.3 (M+H).

### Example 152: (±)-trans-4-Phenyl-N-[3-(phenylamino)phenyl]pyrrolidine-3-carboxamide hydrochloride (Compound I-89)

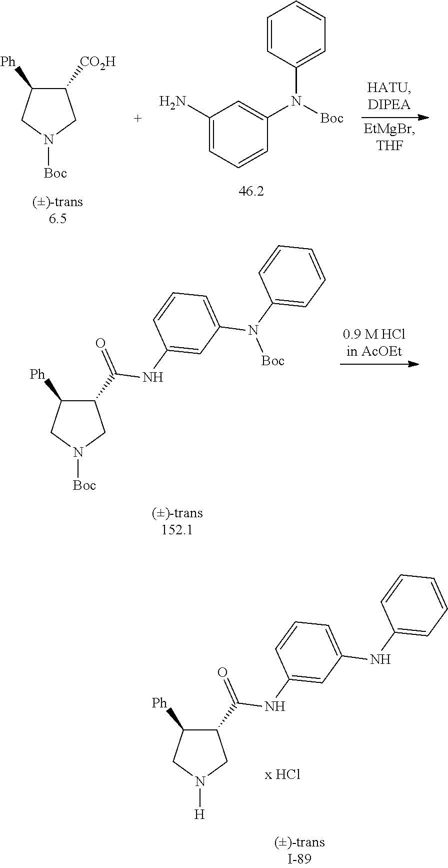 [SMILES: CC(C)(C)OC(=O)N1C[C@@H](C(=O)O)[C@H](c2ccccc2)C1 ; CC(C)(C)OC(=O)N(c1ccccc1)c1cccc(N)c1 ; CC(C)(C)OC(=O)N1C[C@@H](C(=O)Nc2cccc(N(C(=O)OC(C)(C)C)c3ccccc3)c2)[C@H](c2ccccc2)C1 ; O=C(Nc1cccc(Nc2ccccc2)c1)[C@@H]1CNC[C@H]1c1ccccc1]

### Step 1: tert-Butyl (±)-trans-4-phenyl-3-({3-[(tert-butoxycarbonyl)(phenyl)amino]phenyl}carbamoyl) pyrrolidine-1-carboxylate (152.1)

Intermediate 152.1 was prepared according to the procedure described in Step 1 of Example 64 starting from a solution of intermediate 6.5 (137 mg, 0.47 mmol), HATU (232.3 mg, 0.61 mmol), DIPEA (0.25 mL, 1.41 mmol), 3.0 M EtMgBr in Et₂O (0.47 mL, 1.41 mmol), and intermediate 46.2 (160 mg, 0.56 mmol) in THF (5 mL+5 mL). Stirring was continued at r.t. for 16 h. After purification by flash chromatography (PET/EtOAc from 100% PET to 70:30 v/v PET/EtOAc), the intermediate 152.1 (50 mg, 0.089 mmol) was obtained as a white solid. Yield 19%. MS-ESI(+) m/z: 458.3 (M+H).

### Step 2: (±)-trans-4-Phenyl-N-[3-(phenylamino)phenyl]pyrrolidine-3-carboxamide hydrochloride (Compound I-89)

Compound I-89 was prepared following the procedure described in Step 2 of Example 120 starting from a solution of intermediate 152.1 (46 mg, 0.08 mmol) and a 0.9 M solution of HCl in EtOAc (0.9 mL). The title compound I-89 (20 mg, 0.05 mmol) was obtained as a white solid. Yield: 63%. ¹H NMR (400 MHz, DMSO-d₆) δ 3.23-3.43 (m, 3H), 3.68-3.71 (m, 3H), 6.72 (dd, J=8.0 Hz, J=1.3 Hz, 1H), 6.82 (t, J=13 Hz, 1H), 6.97 (d, J=8.8 Hz, 1H), 7.05 (d, J=1.1 Hz, 2H), 7.10 (t, J=8.0 Hz, 1H), 7.22 (t, J=1.9 Hz, 2H), 7.26-7.30 (m, 1H), 7.33-7.41 (m, 5H), 9.5 (brs, 1H), 9.91 (brs, 1H), 10.17 (s, 1H). HPLC purity: ≥95%. MS-ESI(+) m/z: 358.4 (M+H).

### Example 153: (±)-trans-N-[3-(4-Cyanophenoxy)phenyl]-4-phenylpyrrolidine-3-carboxamide hydrochloride (Compound I-90)

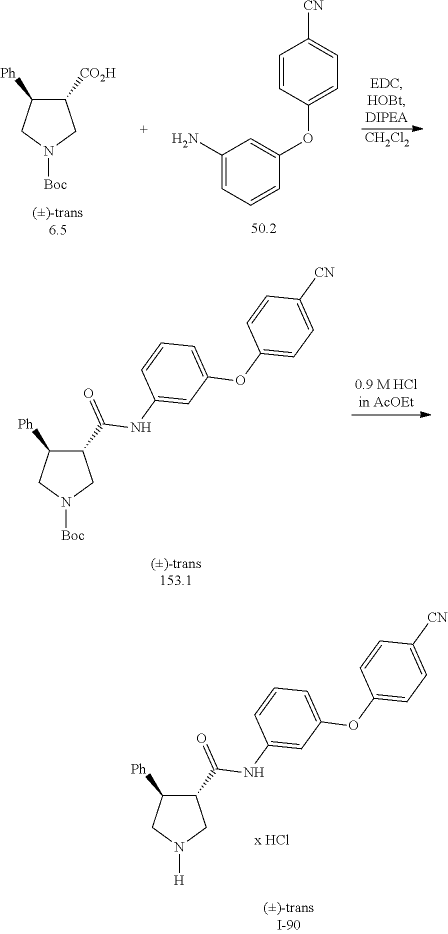

### Step 1: (H-trans tert-Butyl N-[3-(4-cyanophenoxy)phenyl]-4-phenylpyrrolidine-3-carboxamide-1-carboxylate (153.1)

Intermediate 153.1 was prepared according to the procedure described in Step 1 of Example 94 from intermediate 6.5 (250 mg, 0.86 mmol), EDC (247.3 mg, 1.3 mmol), HOBt (176 mg, 1.3 mmol), intermediate 50.2 (214.4 mg, 1.02 mmol), and DIPEA (230 μL, 1.3 mmol) in DCM (15 mL). The intermediate 153.1 (120 mg, 0.24 mmol) was obtained after work-up and chromatographic purification (PET/EtOAc, from 100% PET to 70:30 v/v PET/EtOAc). Yield: 29%. MS-ESI(+) m/z: 484.3 (M+H).

### Step 2: (±)-trans-4-Phenyl-N-[3-(phenylamino)phenyl]pyrrolidine-3-carboxamide hydrochloride (Compound I-90)

Compound I-90 was prepared following the procedure described in Step 2 of Example 120 starting from a solution of intermediate 153.1 (118 mg, 0.24 mmol) and a 0.9 M solution of HCl in EtOAc (3 mL). The title compound I-90 (65 mg, 0.15 mmol) was obtained as a yellowish solid. Yield: 64%. ¹H NMR (400 MHz, DMSO-d₆) δ 3.20-3.35 (m, 1H), 3.42-3.49 (m, 2H), 3.71-3.78 (m, 3H), 6.86 (dt, J=6.5 Hz, J=2.2 Hz, 1H), 7.15-7.10 (m, 2H), 7.29-7.33 (m, 1H), 7.36-7.42 (m, 6H), 7.46 (m, 1H), 7.85-7.88 (m, 2H), 9.48 (brs, 1H), 9.89 (brs, 1H), 10.59 (s, 1H). HPLC purity: ≥95%. MS-ESI(+) m/z: 384.4 (M+H).

### Example 154: (±)-trans-N-[trans-3-(4-Fluorophenoxy)cyclobutyl]-4-phenylpyrrolidine-3-carboxamide hydrochloride (Compound I-91)

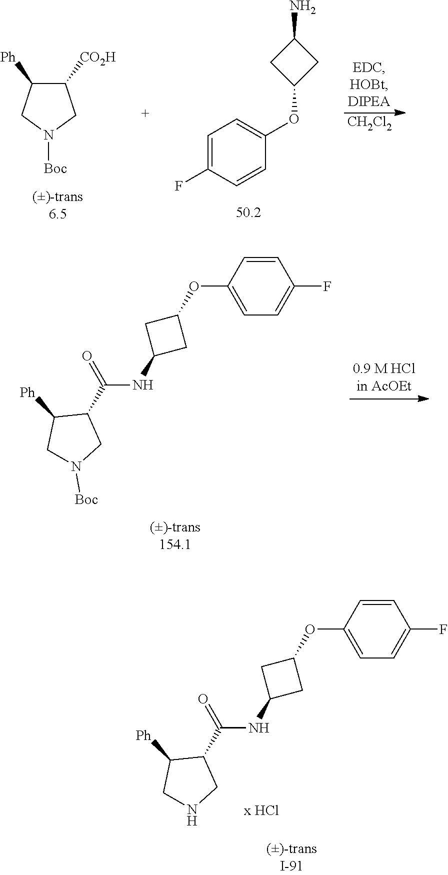 [SMILES: CC(C)(C)OC(=O)N1C[C@@H](C(=O)O)[C@H](c2ccccc2)C1 ; N[C@H]1C[C@H](Oc2ccc(F)cc2)C1 ; "CH2Cl2" ; CC(C)(C)OC(=O)N1C[C@@H](C(=O)N[C@H]2C[C@H](Oc3ccc(F)cc3)C2)[C@H](c2ccccc2)C1 ; O=C(N[C@H]1C[C@H](Oc2ccc(F)cc2)C1)[C@@H]1CNC[C@H]1c1ccccc1]

### Step 1: (±)-tert-Butyl-N-[trans-3-(4-fluorophenoxy)cyclobutyl]-4-phenylpyrrolidine-3-carboxamide-1-carboxylate (154.1)

Intermediate 154.1 was prepared according to the procedure described in Step 1 of Example 94 from intermediate 6.5 (134 mg, 0.46 mmol), EDC (132 mg, 0.69 mmol), HOBt (93 mg, 0.69 mmol), DIPEA (240 μL, 1.38 mmol), and intermediate 60.4 (120 mg, 0.55 mmol) in DCM (10 mL). The intermediate 154.1 (180 mg, 0.4 mmol) was obtained after work-up and chromatographic purification (PET/EtOAc, from 100% PET to 70:30 v/v PET/EtOAc) as a colorless oil. Yield: 86%. MS-ESI(+) m/z: 455.4 (M+H).

### Step 2: (±)-trans-N-[trans-3-(4-Fluorophenoxy)cyclobutyl]-4-phenylpyrrolidine-3-carboxamide hydrochloride (Compound I-91)

Compound I-91 was prepared following the procedure described in Step 2 of Example 120 starting from a solution of intermediate 154.1 (170 mg, 0.37 mmol) and a 0.9 M solution of HCl in EtOAc (3.5 mL). The title compound I-91 (100 mg, 0.26 mmol) was obtained as a yellowish solid. Yield: 69%. ¹H NMR (400 MHz, DMSO-d₆) δ 2.12-2.14 (m, 1H), 2.22-2.33 (m, 3H), 3.08 (m, 1H), 3.25 (t, J=10.4 Hz, 2H), 3.25-3.69 (m, 3H), 4.15-4.23 (m, 1H), 4.61-4.67 (m, 1H), 6.76 (dd, J=6.5 Hz, J=4.2 Hz, 2H), 7.10 (dd, J=12.1 Hz, 2H), 7.27-7.31 (m, 1H), 7.33-7.38 (m, 4H), 8.64 (d, J=6.5 Hz, 1H), 9.50 (brs, 1H), 9.88 (brs, 1H). HPLC purity: ≥95%. MS-ESI(+) m/z: 355.4 (M+H).

### Example 155: (±)-trans-4-Phenyl-N-{3-[4-(trifluoromethyl)phenoxy]phenyl}pyrrolidine-3-carboxamide hydrochloride (Compound I-92)

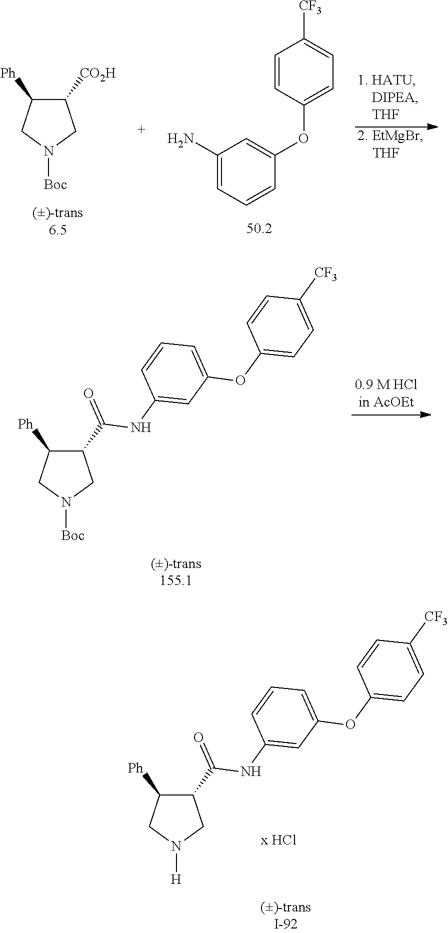 [SMILES: CC(C)(C)OC(=O)N1C[C@@H](C(=O)O)[C@H](c2ccccc2)C1 ; Nc1cccc(Oc2ccc(C(F)(F)F)cc2)c1 ; "2. EtMgBr,    THF" ; CC(C)(C)OC(=O)N1C[C@@H](C(=O)Nc2cccc(Oc3ccc(C(F)(F)F)cc3)c2)[C@H](c2ccccc2)C1 ; O=C(Nc1cccc(Oc2ccc(C(F)(F)F)cc2)c1)[C@@H]1CNC[C@H]1c1ccccc1]

### Step 1: tert-Butyl (±)-trans-4-Phenyl-N-[3-[4-(trifluoromethyl)phenoxy]phenyl]pyrrolidine-3-carboxamide-1-carboxylate (155.1)

Intermediate 155.1 was prepared according to the procedure described in Step 1 of Example 64 starting from intermediate 6.5 (81 mg, 0.28 mmol), HATU (127 mg, 0.33 mmol), DIPEA (0.15 mL, 0.84 mmol), 3.0 M EtMgBr in Et₂O (0.28 mL, 0.84 mmol), and intermediate 58.1 (212 mg, 0.84 mmol) in THF (1.5 mL+1.5 mL). Stirring was continued at r.t. for 16 h. After purification by flash chromatography (DCM/MeOH from 100% DCM to 95:5 v/v DCM/MeOH) the intermediate 155.1 (52 mg, 0.10 mmol) was obtained as a yellowish solid. Yield: 36%. MS-ESI(+) m/z: 527.8 (M+H); MS-ESI(−) m/z: 525.7 (M−H).

### Step 2: (±)-trans-4-Phenyl-N-[3-[4-(trifluoromethyl)phenoxy]phenyl]pyrrolidine-3-carboxamide hydrochloride (Compound I-92)

Compound I-92 was prepared following the procedure described in Step 2 of Example 120 starting from a solution of intermediate 155.1 (52 mg, 0.10 mmol) and a 0.9 M solution of HCl in EtOAc (1.1 mL). The title compound I-92 (47 mg, 0.10 mmol) was obtained as a yellowish solid. Yield: quantitative. (400 MHz, DMSO-d₆) δ 3.22-3.43 (m, 4H), 3.67-3.73 (m, 2H), 6.82 (dd, J1=5.7 Hz, J2=3.4 Hz, 1H), 7.14 (d, J=8.5 Hz, 2H), 7.25-7.29 (m, 1H), 7.32-7.39 (m, 7H), 7.73 (d, J=8.6 Hz, 2H), 9.42 (brs, 1H), 9.83 (brs, 1H), 10.50 (s, 1H). HPLC purity: ≥95%. MS-ESI(+) m/z: 427.4 (M+H).

### Example 156: (±)-trans-N-[3-(4-Fluorophenoxy)phenyl]-4-phenylpyrrolidine-3-carboxamide hydrochloride (Compound I-93)

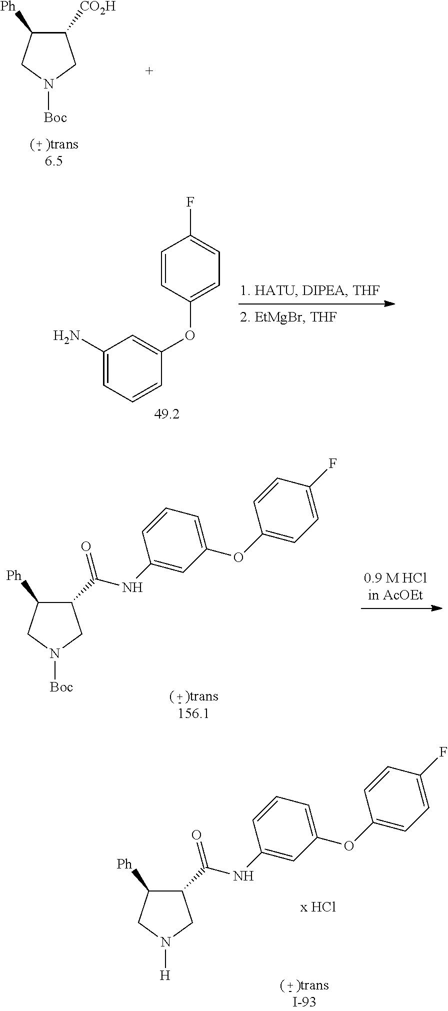

### Step 1: tert-Butyl (±)-trans-N-[3-(4-fluorophenoxy)phenyl]-4-phenylpyrrolidine-3-carboxamide-1-carboxylate (156.1)

Intermediate 156.1 was prepared according to the procedure described in Step 1 of Example 64 starting from intermediate 6.5 (81 mg, 0.28 mmol), HATU (127 mg, 0.33 mmol), DIPEA (0.15 mL, 0.84 mmol), 3.0 M EtMgBr in Et₂O (0.28 mL, 0.84 mmol), and intermediate 49.2 (170 mg, 0.84 mmol) in THF (1.5 mL+1.5 mL). Stirring was continued at r.t. for 16 h. After purification by flash chromatography (DCM/MeOH from 100% DCM to 95:5 v/v DCM/MeOH) the intermediate 156.1 (39 mg, 0.08 mmol) was obtained as a yellowish solid. Yield 29%. MS-ESI(+) m/z: 477.8 (M+H); MS-ESI(−) m/z: 475.6 (M−H).

### Step 2: (±)-trans-N-[3-(4-Fluorophenoxy)phenyl]-4-phenylpyrrolidine-3-carboxamide hydrochloride (Compound I-93)

Compound I-93 was prepared following the procedure described in Step 2 of Example 120 starting from a solution of intermediate 156.1 (39 mg, 0.08 mmol) and a 0.9 M solution of HCl in EtOAc (0.9 mL). The title compound I-93 (33 mg, 0.08 mmol) was obtained as a white solid. Yield: quantitative. (400 MHz, DMSO-d₆) δ 3.11-3.36 (m, 4H), 3.61-3.66 (m, 2H), 6.61-6.64 (m, 1H), 6.98-7.02 (m, 2H), 7.14-7.24 (m, 6H), 7.27-7.32 (4H), 9.36 (brs, 1H), 9.75 (brs, 1H), 10.32 (s, 1H).

HPLC purity: ≥95%. MS-ESI(+) m/z: 377.4 (M+H).

### Example 157: (±)-trans-4-Phenyl-N-[3-(phenylcarbonyl)phenyl]pyrrolidine-3-carboxamide hydrochloride (Compound I-94)

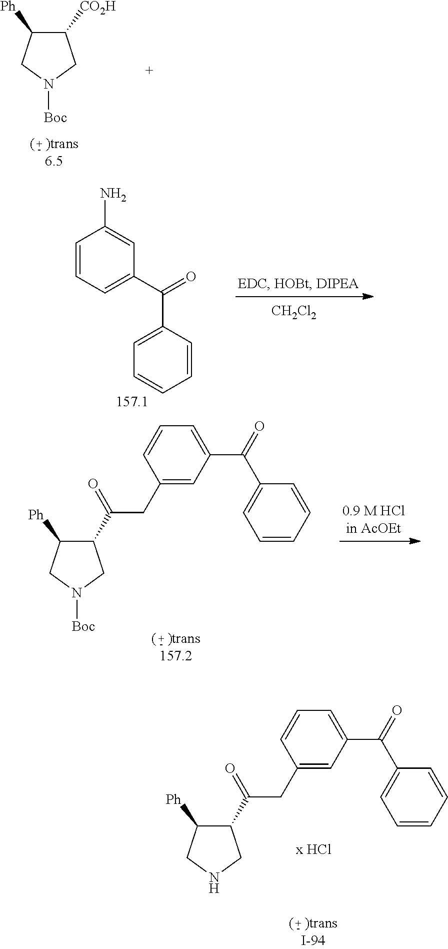

### Step 1: tert-Butyl (±)-trans-4-phenyl-N-[3-(phenylcarbonyl)phenyl]pyrrolidine-3-carboxamide hydrochloride-1-carboxylate (157.2)

Intermediate 157.2 was prepared according to the procedure described in Step 1 of Example 94 from intermediate 6.5 (200 mg, 0.69 mmol), intermediate 157.1 (135 mg, 0.69 mmol), EDC (107 mg, 0.69 mmol), HOBt (93 mg, 0.69 mmol), and DIPEA (0.18 mL, 1.0 mmol) in DCM (5 mL). Stirring was continued for 3 days. The intermediate 157.2 (38 mg, 0.08 mmol) was obtained after work-up and chromatographic purification (PET/EtOAc, from 100% PET to 1:1 v/v PET/EtOAc) as a yellow oil. Yield: 12%. MS-ESI(−) m/z: 469.5 (M−H).

### Step 2: (±)-trans-4-Phenyl-N-[3-(phenylcarbonyl)phenyl]pyrrolidine-3-carboxamide hydrochloride (Compound I-94)

Compound I-94 was prepared following the procedure described in Step 2 of Example 120 starting from a solution of intermediate 157.2 (38 mg, 0.08 mmol) and a 0.9 M solution of HCl in EtOAc (0.9 mL). The title compound I-94 (30 mg, 0.07 mmol) was obtained as a brownish solid. Yield: 88%. (400 MHz, DMSO-d₆) δ 3.28-3.44 (m, 4H), 3.70-3.77 (m, 2H), 7.27-7.31 (m, 1H), 7.34-7.42 (m, 5H), 7.48 (t, J=7.8 Hz, 1H), 7.56 (m, 2H), 7.67-7.72 (m, 3H), 7.85 (ddd, J1=12.8 Hz, J2=6.8 Hz, J3=1.2 Hz, 1H), 7.97 (t, J=7.8 Hz, 1H), 9.39 (brs, 1H), 9.73 (brs, 1H), 10.54 (s, 1H). HPLC purity: ≥90%. MS-ESI(+) m/z: 371.4 (M+H).

### Example 158: (3S,4R)—N-(Isoquinolin-5-ylmethyl)-4-phenylpyrrolidine-3-carboxamide dihydrochloride (Compound I-95)

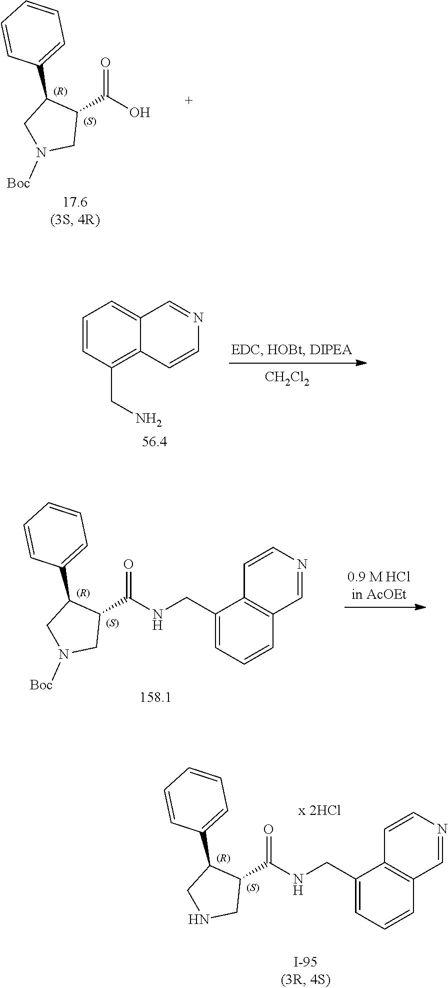

### Step 1: tert-Butyl (3S,4R)—N-(isoquinolin-5-ylmethyl)-4-phenylpyrrolidine-3-carboxamide-1-carboxylate (158.1)

Intermediate 158.1 was prepared according to the procedure described in Step 1 of Example 94 from intermediate 17.6 (200 mg, 0.68 mmol), EDC (197 mg, 1.03 mmol), HOBt (139.2 mg, 1.03 mmol), DIPEA (0.36 μL, 2.04 mmol), and intermediate 56.4 (129 mg, 0.82 mmol) in DCM (15 mL). The intermediate 158.1 (150 mg, 0.34 mmol) was obtained after work-up and chromatographic purification (DCM/MeOH, from 100% DCM to 95:5 v/v DCM/MeOH) as a colorless oil. Yield: 51%. MS-ESI(+) m/z: 432.2 (M+H).

### Step 2: (3S,4R)—N-(Isoquinolin-5-ylmethyl)-4-phenylpyrrolidine-3-carboxamide dihydrochloride (Compound I-95)

Compound I-95 was prepared following the procedure described in Step 2 of Example 120 starting from a solution of intermediate 158.1 (100 mg, 0.23 mmol) and a 0.9 M solution of HCl in EtOAc (2.6 mL). The title compound I-95 (80 mg, 0.19 mmol) was obtained as a yellowish solid. Yield: 86%. ¹H NMR (400 MHz, DMSO-d₆) δ 3.16-3.29 (m, 3H), 3.53-3.65 (m, 3H), 4.61 (dd, J=15.6 Hz, J=5.1 Hz, 1H), 4.86 (dd, J=15.7 Hz, J=6.4 Hz, 1H), 7.23-7.32 (m, 5H), 7.59 (d, J=6.5 Hz, 1H), 7.78 (dd, J=8.2 Hz, J=7.2 Hz, 1H), 8.36 (dd, J=11.0, 7.5 Hz, 2H), 8.64 (d, J=6.6 Hz, 1H), 9.00 (t, J=5.7 Hz, 2H), 9.72 (brs, 1H), 9.83 (s, 1H), 10.01 (brs, 1H). HPLC purity: ≥95%. MS-ESI(+) m/z: 332.3 (M+H).

### Example 159: (3R,4S)—N-(1-Methylisoquinolin-5-yl)-4-[4-(trifluoromethyl)phenyl]pyrrolidine-3-carboxamide dihydrochloride (Compound I-96)

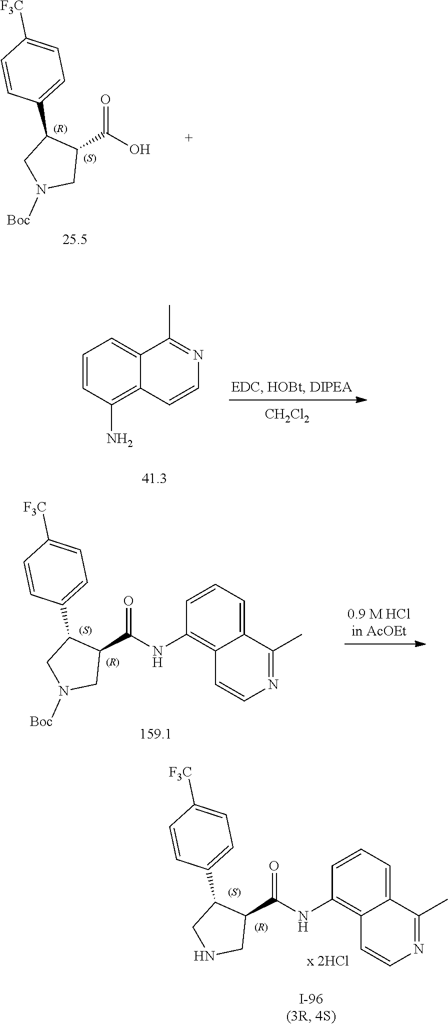

### Step 1: tert-Butyl (3R,4S)—N-(1-methylisoquinolin-5-yl)-4-[4-(trifluoromethyl)phenyl]pyrrolidine-3-carboxamide-1-carboxylate (159.1)

Intermediate 159.1 was prepared according to the procedure described in Step 1 of Example 64 starting from a solution of intermediate 25.5 (246 mg, 0.68 mmol), HATU (336.1 mg, 0.88 mmol), DIPEA (0.36 mL, 2.04 mmol), 3.0 M EtMgBr in Et₂O (0.68 mL, 2.04 mmol), and intermediate 41.3 (130 mg, 0.82 mmol) in THF (5 mL+5 mL). Stirring was continued at r.t. for 16 h. After purification by flash chromatography (DCM/MeOH, from 100% DCM to 95:5 v/v DCM/MeOH), the intermediate 159.1 (70 mg, 0.14 mmol) was obtained as a colorless oil. Yield 21%. MS-ESI(+) m/z: 500.2 (M+H).

### Step 2: (3R,4S)—N-(1-Methylisoquinolin-5-yl)-4-[4-(trifluoromethyl)phenyl]pyrrolidine-3-carboxamide dihydrochloride (Compound I-96)

Compound I-96 was prepared following the procedure described in Step 2 of Example 120 starting from a solution of intermediate 159.1 (70 mg, 0.14 mmol) and a 0.9 M solution of HCl in EtOAc (1.7 mL). The title compound I-96 (40 mg, 0.084 mmol) was obtained as a yellowish solid. Yield: 60%. ¹H NMR (400 MHz, DMSO-d₆) δ 3.0+9 (s, 3H), 3.30-3.41 (m, 2H), 3.70-3.80 (m, 5H), 7.65 (d, J=8.3 Hz, 2H), 7.69 (d, J=8.4 Hz, 2H), 7.91-7.82 (m, 2H), 8.09 (d, J=7.5 Hz, 1H), 8.27 (d, J=6.8 Hz, 1H), 8.31 (d, J=8.5 Hz, 1H), 9.77 (brs, 1H), 10.11 (brs, 1H), 10.8 (s, 1H). HPLC purity: ≥95%. MS-ESI(+) m/z: 400.3 (M+H).

### Example 160: (±)-trans-4-Phenyl-N-[3-(3-methylphenoxy)phenyl]pyrrolidine-3-carboxamide hydrochloride (Compound I-97)

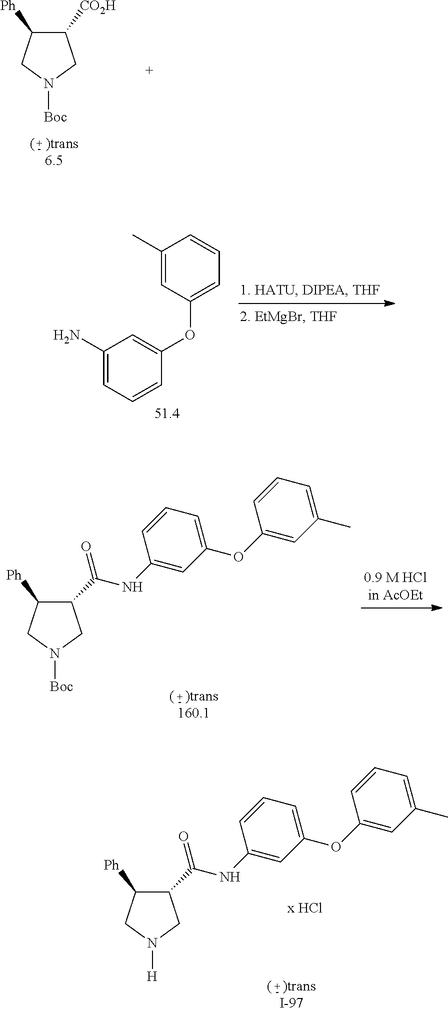

### Step 1: (±)-trans tert-Butyl-4-phenyl-N-[3-(3-methylphenoxy)phenyl]pyrrolidine-3-carboxamide-1-carboxylate (160.1)

Intermediate 160.1 was prepared according to the procedure described in Step 1 of Example 64 starting from a solution of intermediate 6.5 (200 mg, 0.69 mmol), HATU (339 mg, 0.89 mmol), DIPEA (0.36 mL, 2.1 mmol), 3.0 M EtMgBr in Et₂O (0.69 mL, 2.1 mmol), and intermediate 51.4 (179 mg, 0.9 mmol) in THF (5 mL+5 mL). Stirring was continued at r.t. for 16 h. After purification by flash chromatography (PET/EtOAc, from 100% PET to 80:20 v/v PET/EtOAc) the intermediate 160.1 (60 mg, 0.13 mmol) was obtained as a colorless oil. Yield 18%. MS-ESI(+) m/z: 473.1 (M+H).

### Step 2: (±)-trans-4-Phenyl-N-[3-(3-methylphenoxy)phenyl]pyrrolidine-3-carboxamide hydrochloride (Compound I-97)

Compound I-97 was prepared following the procedure described in Step 2 of Example 120 starting from a solution of intermediate 160.1 (40 mg, 0.084 mmol) and a 0.9 M solution of HCl in EtOAc (1 mL). The title compound I-97 (20 mg, 0.048 mmol) was obtained as a yellowish solid. Yield: 58%. ¹H NMR (400 MHz, DMSO-d₆) δ 2.29 (s, 3H), 3.27-3.42 (m, 4H), 3.70-3.73 (m, 3H), 6.68-6.71 (m, 1H), 6.77-6.80 (m, 1H), 6.83 (m, 1H), 6.95-6.99 (m, 1H), 7.25-7.27 (m, 2H), 7.29-7.32 (m, 2H), 7.35-7.38 (m, 4H), 9.43 (brs, 1H), 9.82 (brs, 1H), 10.38 (s, 1H).

HPLC purity: ≥95%. MS-ESI(+) m/z: 373.3 (M+H).

### Example 161: (±)-trans-4-Phenyl-N-[3-(pyridin-4-yloxy)phenyl]pyrrolidine-3-carboxamide dihydrochloride (Compound I-98)

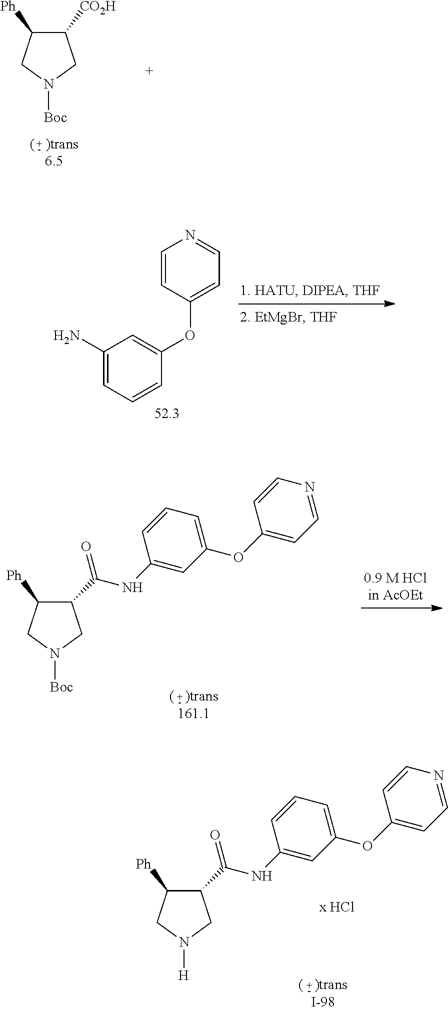

### Step 1: tert-Butyl (±)-trans-4-phenyl-N-[3-(pyridin-4-yloxy)phenyl]pyrrolidine-3-carboxamide-1-carboxylate (134.2)

Intermediate 161.1 was prepared according to the procedure described in Step 1 of Example 64 starting from intermediate 6.5 (150 mg, 0.51 mmol), HATU (235 mg, 0.62 mmol), DIPEA (0.27 mL, 1.54 mmol), 3.0 M EtMgBr in Et₂O (0.52 mL, 1.54 mmol), and intermediate 52.3 (144 mg, 0.77 mmol) in THF (2.5 mL+2.5 mL). Stirring was continued at r.t. for 16 h. After purification by flash chromatography (DCM/MeOH from 100% DCM to 9:1 v/v DCM/MeOH) the intermediate 161.1 (14 mg, 0.03 mmol) was obtained. Yield: 5%. MS-ESI(+) m/z: 460.8 (M+H); MS-ESI(−) m/z: 458.8 (M−H).

### Step 2: (±)-trans-4-Phenyl-N-[3-(pyridin-4-yloxy)phenyl]pyrrolidine-3-carboxamide dihydrochloride (Compound I-98)

Compound I-98 was prepared following the procedure described in Step 2 of Example 120 starting from a solution of intermediate 161.1 (14 mg, 0.030 mmol) and a 0.9 M solution of HCl in EtOAc (0.3 mL). The title compound I-98 (12 mg, 0.028 mmol) was obtained as a pale yellow solid. Yield: 93%. (400 MHz, DMSO-d₆) δ 3.31-3.43 (m, 2H), 3.54 (dd, J1=17.9 Hz, J2=9.1 Hz, 1H), 3.76-3.82 (m, 3H), 7.10 (brs, 2H), 7.30-7.34 (m, 1H), 7.37-7.44 (m, 5H), 7.57 (t, J=8.1 Hz, 1H), 7.67 (d, J=8.8 Hz, 1H), 8.01 (s, 1H), 8.55 (brs, 2H), 9.53 (brs, 1H), 9.88 (brs, 1H), 10.85 (s, 1H). HPLC purity: ≥95%. MS-ESI(+) m/z: 360.3 (M+H).

### Example 162: (±)-trans-4-Phenyl-N-[3-(pyridin-2-yloxy)phenyl]pyrrolidine-3-carboxamide dihydrochloride (Compound I-99)

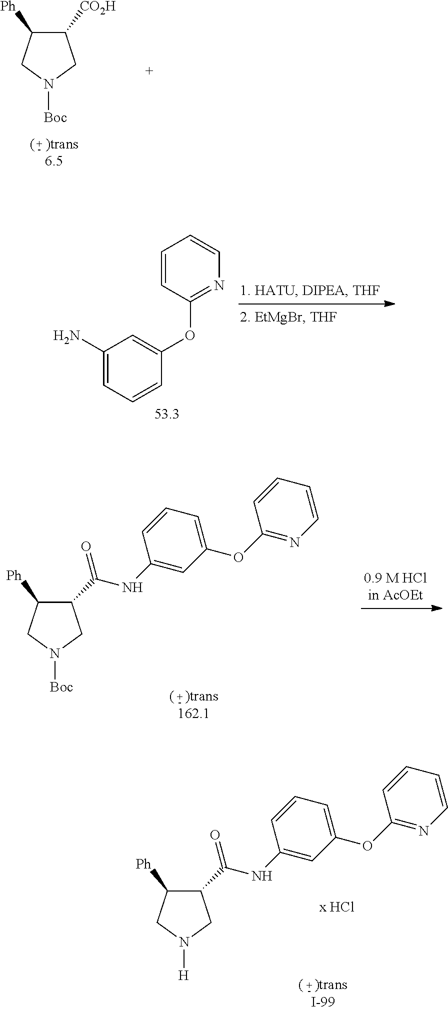

### Step 1: tert-Butyl (±)-trans-4-phenyl-N-[3-(pyridin-2-yloxy)phenyl]pyrrolidine-3-carboxamide-1-carboxylate (162.1)

Intermediate 162.1 was prepared according to the procedure described in Step 1 of Example 64 starting from intermediate 6.5 (96 mg, 0.33 mmol), HATU (149 mg, 0.39 mmol), DIPEA (0.17 mL, 0.98 mmol), 3.0 M EtMgBr in Et₂O (0.22 mL, 0.66 mmol), and intermediate 53.3 (61 mg, 0.33 mmol) in THF (1.5 mL+1.5 mL). Stirring was continued at r.t. for 16 h. After purification by flash chromatography (DCM/MeOH from 100% DCM to 9:1 v/v DCM/MeOH) the intermediate 162.1 (52 mg, 0.11 mmol) was obtained. Yield: 33%. MS-ESI(+) m/z: 460.6 (M+H); MS-ESI(−) m/z: 458.8 (M−H).

### Step 2: (±)-trans-4-Phenyl-N-[3-(pyridin-2-yloxy)phenyl]pyrrolidine-3-carboxamide dihydrochloride (Compound I-99)

Compound I-99 was prepared following the procedure described in Step 2 of Example 120 starting from a solution of intermediate 162.1 (52 mg, 0.11 mmol) and a 0.9 M solution of HCl in EtOAc (1.2 mL). The title compound I-99 (46 mg, 0.11 mmol) was obtained as a pale yellow solid. Yield: quantitative. (400 MHz, DMSO-d₆) δ 3.25-3.47 (m, 4H), 3.69-3.77 (m, 2H), 6.30 (td, J1=6.7 Hz, J2=1.3 Hz, 1H), 6.47 (d, J=12 Hz, 1H), 7.07 (ddd, J1=7.9 Hz, J2=2.0 Hz, J3=0.9 Hz, 1H), 7.25-7.31 (m, 1H), 7.33-7.45 (m, 5H), 7.47-7.53 (m, 1H), 7.53-7.57 (m, 1H), 7.57-7.60 (m, 1H), 7.68 (t, J=2.0 Hz, 1H), 9.42 (brs, 1H), 9.78 (brs, 1H), 10.60 (s, 1H). HPLC purity: ≥95%. MS-ESI(+) m/z: 360.3 (M+H).

### Example 163: (±)-trans Isoquinolin-5-yl[(3S,4R)-4-phenylpyrrolidin-3-yl]methanone dihydrochloride (Compound I-100)

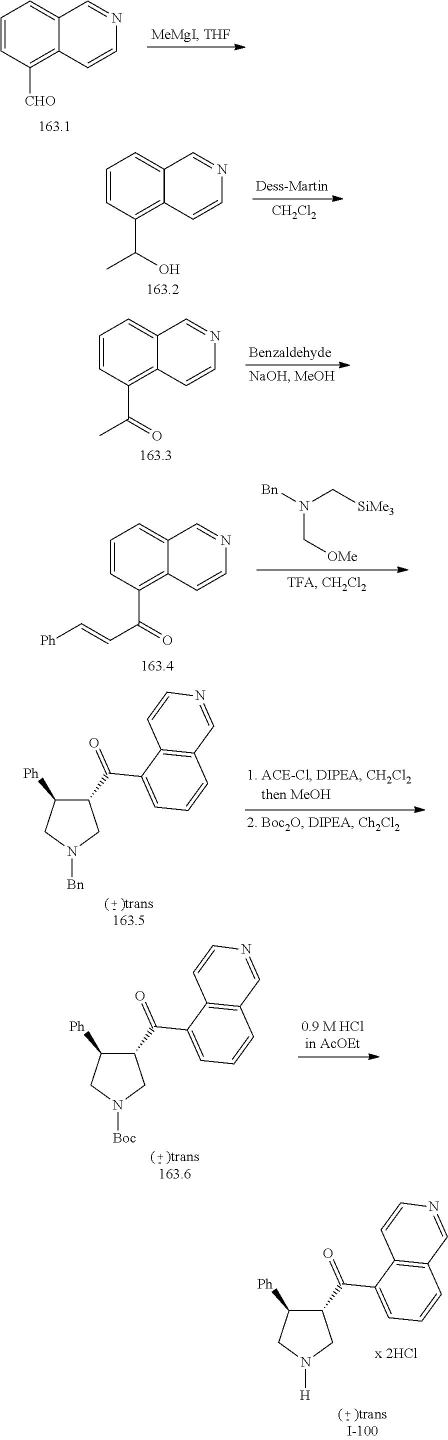

### Step 1: 1-(Isoquinolin-5-yl)ethanol (163.2)

A solution of 3.0 M MeMgI in Et₂O (5.3 mL, 15.91 mmol) under N₂ atmosphere was diluted with Et₂O (5 mL) and cooled to −10° C. A solution of 163.1 (1.0 g, 6.36 mmol) in THF (20 mL) was then added dropwise and the resulting mixture was reacted under magnetic stirring at r.t. for 1 h. The mixture was poured into H₂O (30 mL) and extracted with EtOAc (3×10 mL). The combined organic layers were washed with H₂O (20 mL) and brine (20 mL), dried over anhydrous Na₂SO₄, and evaporated to dryness. Purification by flash chromatography (PET/EtOAc, from 93:7 v/v to 7:3 v/v) gave the desired intermediate 163.2 (865 mg, 4.99 mmol) as a brown oil. Yield: 79%. MS-ESI(+) m/z: 174.3 (M+H).

### Step 2: 1-(Isoquinolin-5-yl)ethanone (163.3)

Dess-Martin reagent (2.74 g, 6.45 mmol) was added to a stirred solution of 163.2 (860 mg, 4.96 mmol) in DCM (15 mL) and the resulting solution was reacted under magnetic stirring at r.t. for 18 h. The whitish suspension thus obtained was poured into DCM (50 mL), then washed with H₂O (2×30 mL) and brine (30 mL). The combined organic layers were dried over anhydrous Na₂SO₄, and evaporated to dryness. Purification by flash chromatography (DCM/MeOH, from 100% DCM to 95:5 v/v DCM/MeOH) gave the desired intermediate 163.3 (835 mg, 4.88 mmol). Yield: 98%. MS-ESI(+) m/z: 172.3 (M+H).

### Step 3: (2E)-1-(Isoquinolin-5-yl)-3-phenylprop-2-en-1-one (163.4)

Intermediate 163.3 (830 mg, 4.85 mmol) was dissolved in MeOH (25 mL), NaOH (582 mg, 14.54 mmol) and benzaldehyde (0.54 mL, 5.33 mmol) were then added sequentially. The resulting mixture was reacted under magnetic stirring for 18 h. The volatiles were then removed under vacuum and the residue poured into 0.5 M aq. HCl (15 mL) then extracted with EtOAc (2×15 mL). The aqueous phase was basified with 2.0 M aq. NaOH and extracted with DCM (3×15 mL) The combined organic layers were washed with brine (20 mL), dried over anhydrous Na₂SO₄, and evaporated to dryness. Purification by flash chromatography (DCM/MeOH, from 100% DCM to 96:4 v/v DCM/MeOH) gave the desired intermediate 163.4 (75 mg, 0.29 mmol). Yield: 6%. MS-ESI(+) m/z: 260.5 (M+H).

### Step 4: [(±)-trans-1-Benzyl-4-phenylpyrrolidin-3-yl](isoquinolin-5-yl)methanone (163.5)

Intermediate 163.5 was synthesized according to the procedure described in Step 1 of Example 1 from intermediate 163.4 (75 mg, 0.29 mmol), intermediate 1.2 (0.11 mL, 0.43 mmol), and TFA (0.011 mL, 0.14 mmol) in DCM (10 mL). Stirring was continued for 24 h. After workup, the crude reaction mixture was used such as for the next step. MS-ESI(+) m/z: 393.8 (M+H).

### Step 5: tert-Butyl (±)-trans-3-(isoquinolin-5-ylcarbonyl)-4-phenylpyrrolidine-1-carboxylate (163.6)

Intermediate 163.6 was synthesized according to the procedure described in Step 2 of Example 1 starting from intermediate 163.5 (crude from Step 4, 0.26 mmol), DIPEA (0.06 mL, 0.32 mmol), and 1-chloroethylchloroformate (0.08 mL, 0.72 mmol) in DCM (10 mL). The obtained crude was treated in refluxing MeOH (10 mL). After removal of volatiles, the debenzylated intermediate was reacted with Boc₂O (126 mg, 0.58 mmol) and DIPEA (0.15 mL, 0.87 mmol) in DCM (10 mL). Stirring was continued for 16 h. After purification by flash chromatography (DCM/MeOH, from 100% DCM to 95:5 v/v DCM/MeOH) the desired intermediate 163.6 (10 mg, 0.025 mmol) was obtained. Yield: 9% over three steps. MS-ESI(+) m/z: 403.8 (M+H).

### Step 6: Isoquinolin-5-yl[(3S,4R)-4-phenylpyrrolidin-3-yl]methanone dihydrochloride (Compound I-100)

Compound I-100 was prepared following the procedure described in Step 2 of Example 120 starting from a solution of intermediate 163.6 (10 mg, 0.025 mmol) and a 0.9 M solution of HCl in EtOAc (0.3 mL). The title compound I-100 (8 mg, 0.21 mmol) was obtained as a yellowish solid. Yield: 85%. (400 MHz, DMSO-d₆) δ 3.33-3.44 (m, 1H), 3.67-3.90 (m, 5H), 7.1-7.21 (m, 3H), 7.28 (m, 2H), 7.76 (t, 3=1.1 Hz, 1H), 8.21 (d, J=7.3 Hz, 1H), 8.47 (d, J=8.3 Hz, 1H), 8.60 (d, J=6.3 Hz, 1H), 8.73 (d, J=6.3 Hz, 1H), 9.62-9.70 (m, 2H), 9.80 (brs, 1H). HPLC purity: ≥90%. MS-ESI(+) m/z: 303.2 (M+H).

### Example 164: (±)-trans-N-[trans-3-(Pyridyn-3-yloxy)cyclobutyl]-4-phenylpyrrolidine-3-carboxamide dihydrochloride (Compound I-101)

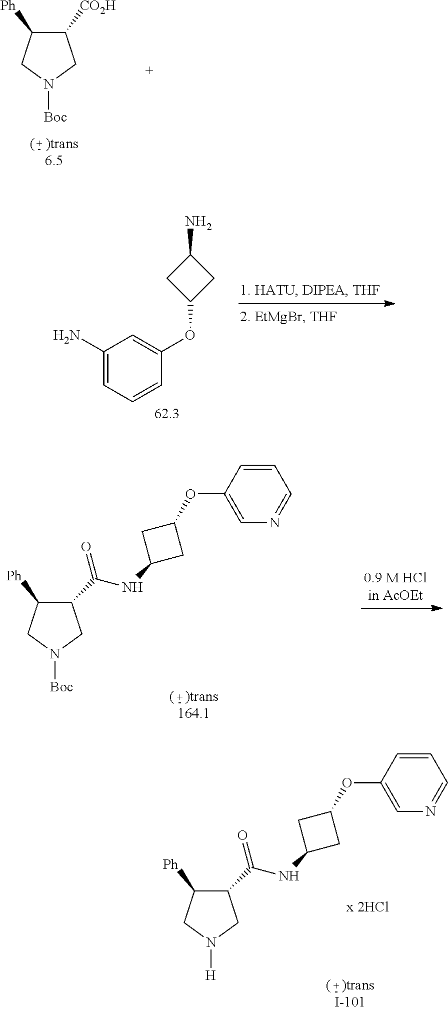

### Step 1: tert-Butyl-(±)-trans-4-phenyl-N-[trans-3-(pyridin-3-yloxy)cyclobutyl]pyrrolidine-3-carboxamide-1-carboxylate (164.1)

Intermediate 164.1 was prepared according to the procedure described in Example 94 from intermediate 6.5 (150 mg, 0.51 mmol), EDC (148 mg, 0.77 mmol), HOBt (104 mg, 0.77 mmol), DIPEA (0.36 μL, 2.04 mmol), and intermediate 62.3 (133 mg, 0.56 mmol) in DCM (10 mL). The intermediate 164.1 (200 mg, 0.46 mmol) was obtained after work-up and chromatographic purification NH-based silica (PET/EtOAc, from 100% PET to 10:90 v/v PET/EtOAc) as a colorless oil. Yield: 89%. MS-ESI(+) m/z: 438.1 (M+H).

### Step 2: (±)-trans-N-[trans-3-(Pyridyn-3-yloxy)cyclobutyl]-4-phenylpyrrolidine-3-carboxamide dihydrochloride (Compound I-101)

Compound I-101 was prepared following the procedure described in Step 2 of Example 120 starting from a solution of intermediate 164.1 (200 mg, 0.46 mmol) and a 0.9 M solution of HCl in EtOAc (5.7 mL). The title compound I-101 (100 mg, 0.24 mmol) was obtained as a white solid. Yield: 53%. ¹H NMR (400 MHz, DMSO-d₆) δ 2.21-2.25 (m, 1H), 2.31-2.39 (m, 2H), 2.43-2.48 (m, 1H), 3.10-3.17 (m, 1H), 3.21-3.25 (m, 2H), 3.56-3.67 (m, 3H), 4.22-4.29 (m, 1H), 4.95-4.96 (m, 1H), 7.26-7.29 (m, 1H), 7.33-7.38 (m, 4H), 7.86-7.93 (m, 2H), 8.47 (d, J=3.2 Hz, 2H), 8.79 (d, J=6.9 Hz, 1H), 9.70 (brs, 1H), 10.07 (brs, 1H). HPLC purity: ≥95%. MS-ESI(+) m/z: 338.2 (M+H).

### Example 165: (±)-trans-N-{trans-3-[(6-Methylpyridin-3-yl)oxy]cyclobutyl}-4-phenylpyrrolidine-3-carboxamide dihydrochloride (Compound I-102)

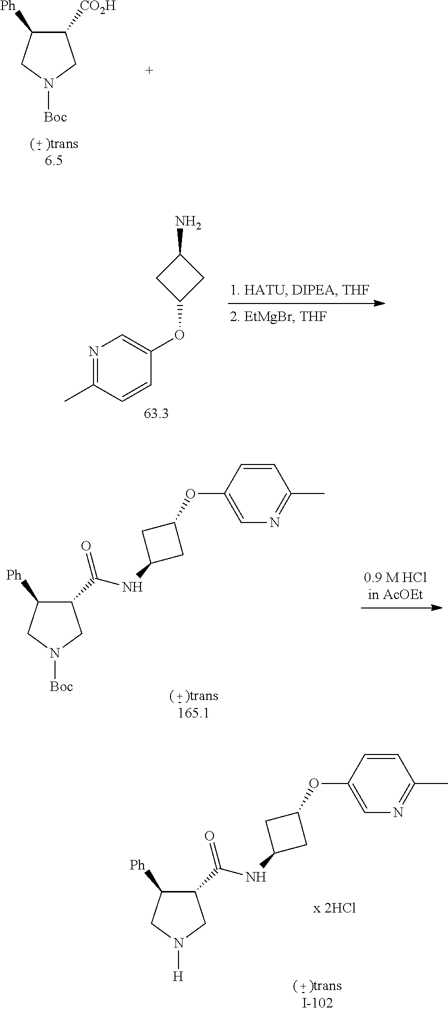

### Step 1: tert-Butyl-(±)-trans-4-phenyl-N-[trans-3-[(6-methylpyridin-3-yl)oxy]cyclobutyl]pyrrolidine-3-carboxamide-1-carboxylate (165.1)

Intermediate 165.1 was prepared according to the procedure described in Step 1 of Example 94 from intermediate 6.5 (150 mg, 0.51 mmol), EDC (148 mg, 0.77 mmol), HOBt (104 mg, 0.77 mmol), DIPEA (0.36 μL, 2.04 mmol), and intermediate 63.3 (140 mg, 0.56 mmol) in DCM (10 mL). The intermediate 165.1 (200 mg, 0.44 mmol) was obtained after work-up and chromatographic purification (DCM/MeOH, from 100% DCM to 96:4 v/v DCM/MeOH) as a white solid. Yield: 87%. MS-ESI(+) m/z: 452.1 (M+H).

### Step 2: (±)-trans-N-[trans-3-(Pyridyn-3-yloxy)cyclobutyl]-4-phenylpyrrolidine-3-carboxamide dihydrochloride (Compound I-101)

Compound I-102 was prepared following the procedure described in Step 2 of Example 120 starting from a solution of intermediate 165.1 (200 mg, 0.46 mmol) and a 0.9 M solution of HCl in EtOAc (3.4 mL). The title compound I-102 (100 mg, 0.23 mmol) was obtained as a white solid. Yield: 51%. ¹H NMR (400 MHz, DMSO-d₆) δ 2.20-2.26 (m, 1H), 2.29-2.37 (m, 2H), 2.40-2.46 m, 1H), 2.65 (m, 3H), 3.10-3.19 (m, 1H), 3.20-3.27 (m, 2H), 3.56-3.69 (m, 3H), 4.22-4.30 (m, 1H), 4.92-4.97 (m, 1H), 7.26-7.33 (m, 1H), 7.33-7.38 (m, 4H), 7.80 (d, J=8.9 Hz, 1H), 7.96 (dd, J=8.9 Hz, J=2.8 Hz, 1H), 8.27 (d, J=2.8 Hz, 1H), 8.79 (d, J=6.9 Hz, 1H), 9.69 (brs, 1H), 10.07 (brs, 1H).

HPLC purity: ≥95%. MS-ESI(+) m/z: 352.2 (M+H).

### Example 166: 3S,4R)-4-Phenyl-N-{4-[6-(trifluoromethyl)pyridin-3-yl]phenyl}pyrrolidine-3-carboxamide hydrochloride_(Compound I-103)

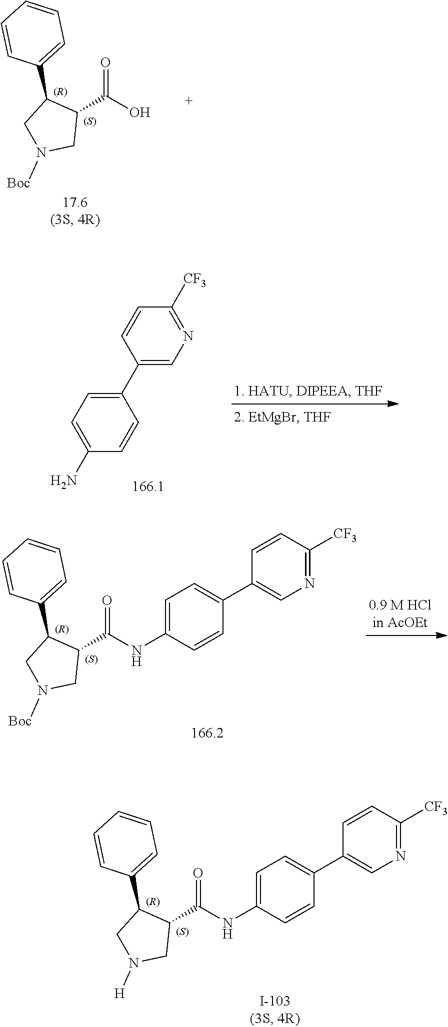

### Step 1: tert-Butyl (3S,4R)-4-phenyl-N-{4-[6-(trifluoromethyl)pyridin-3-yl]phenyl}4-phenylpyrrolidine-3-carboxamide-1-carboxylate (166.2)

Intermediate 166.2 was prepared according to the procedure described in Step 1 of Example 64 starting from a solution of intermediate 17.6 (200 mg, 0.69 mmol), HATU (339 mg, 0.89 mmol), DIPEA (0.36 mL, 2.1 mmol), 3.0 M EtMgBr in Et₂O (0.69 mL, 2.1 mmol), and intermediate 166.1 (198 mg, 0.82 mmol) in THF (5 mL+5 mL). Stirring was continued at r.t. for 16 h. After purification by flash chromatography (PET/EtOAc, from 100% PET to 60:40 v/v PET/EtOAc) the intermediate 166.2 (60 mg, 0.12 mmol) was obtained as a colorless oil. Yield 17%. MS-ESI(+) m/z: 512.1 (M+H).

### Step 2: (3S,4R)-4-Phenyl-N-[4-[6-(trifluoromethyl)pyridin-3-yl]phenyl]pyrrolidine-3-carboxamide hydrochloride (Compound I-103)

Compound I-103 was prepared following the procedure described in Step 2 of Example 120 starting from a solution of intermediate 166.2 (50 mg, 0.097 mmol) and a 0.9 M solution of HCl in EtOAc (1.22 mL). The title compound I-103 (35 mg, 0.05 mmol) was obtained as a white solid. Yield: Yield: 53%. ¹H NMR (400 MHz, DMSO-d₆) δ 3.30-3.49 (m, 4H), 3.73-3.77 (m, 3H), 7.26-7.30 (m, 1H), 7.34-7.40 (m, 4H), 7.73 (d, J=8.6 Hz, 2H), 7.78 (d, J=8.6 Hz, 2H), 7.94 (d, J=8.2 Hz, 1H), 8.31 (d, J=8.6 Hz, 1H), 9.05 (s, 1H), 9.48 (brs, 1H), 9.84 (brs, 1H), 10.56 (s, 1H).

HPLC purity: >95%. MS-ESI(+) m/z: 412.2 (M+H).

### Example 167: (±)-trans-4-Phenyl-N-(3-{[6-(trifluoromethyl)pyridin-3-yl]oxy}phenyl) pyrrolidine-3-carboxamide dihydrochloride (Compound I-104)

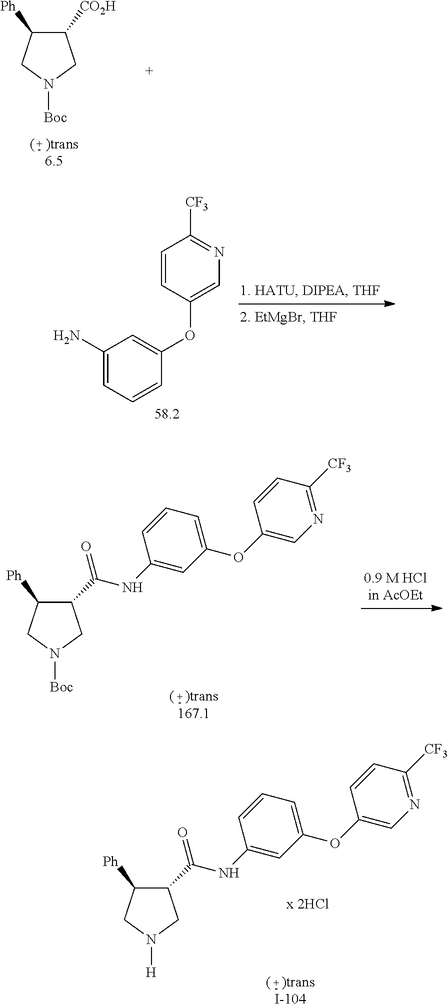

### Step 1: tert-Butyl (±)-trans-4-phenyl-N-(3-{[6-(trifluoromethyl)pyridin-3-yl]oxy}phenyl) pyrrolidine-3-carboxamide-1-carboxylate (167.1)

Intermediate 167.1 was prepared according to the procedure described in Step 1 of Example 64 starting from intermediate 6.5 (118 mg, 0.41 mmol), HATU (185 mg, 0.49 mmol), DIPEA (0.21 mL, 1.22 mmol), 3.0 M EtMgBr in Et₂O (0.16 mL, 0.49 mmol), and intermediate 58.2 (124 mg, 0.49 mmol) in THF (1.8 mL+1.8 mL). Stirring was continued at r.t. for 16 h. After purification by flash chromatography (DCM/MeOH from 100% DCM to 9:1 v/v DCM/MeOH) the intermediate 167.1 (186 mg, 0.35 mmol) was obtained as a yellowish solid. Yield: 86%. MS-ESI(−) m/z: 526.1 (M−H).

### Step 2: (±)-trans-4-Phenyl-N-(3-{[6-(trifluoromethyl)pyridin-3-yl]oxy}phenyl) pyrrolidine-3-carboxamide dihydrochloride (Compound I-104)

Compound I-104 was prepared following the procedure described in Step 2 of Example 120 starting from a solution of intermediate 167.1 (186 mg, 0.35 mmol) and a 0.9 M solution of HCl in EtOAc (3.9 mL). The title compound I-104 (149 mg, 0.30 mmol) was obtained as a pale yellow solid. Yield: 85%. (400 MHz, DMSO-d₆) δ 3.19-3.26 (m, 2H), 3.34-3.40 (m, 1H), 3.63-3.69 (m, 3H), 6.81-6.84 (m, 1H), 7.22-7.36 (m, 7H), 7.41 (d, J=1.8 Hz, 1H), 7.50 (d, J=8.5 Hz, 1H), 7.85 (dd, J1=8.7 Hz, J2=1.8 Hz, 1H), 8.49 (s, 1H), 9.44 (brs, 1H), 9.84 (brs, 1H), 10.54 (s, 1H). HPLC purity: ≥95%. MS-ESI(+) m/z: 428.1 (M+H).

### Example 168: (±)-trans-4-Phenyl-N-{3-[(6-methylpyridin-3-yl)oxy]phenyl}pyrrolidine-3-carboxamide dihydrochloride (Compound I-105)

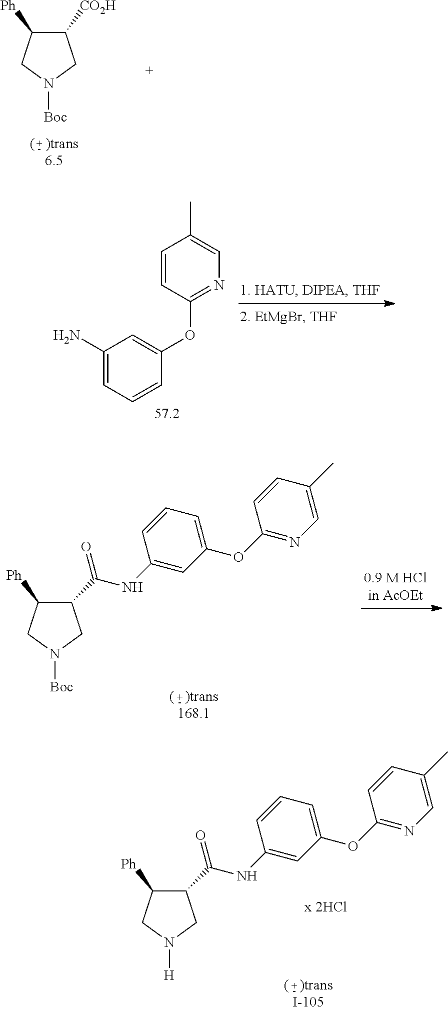

### Step 1: tert-Butyl (±)-trans-4-phenyl-N-[3-[(6-methylpyridin-3-yl)oxy]phenyl]pyrrolidine-3-carboxamide-1-carboxylate (168.1)

Intermediate 168.1 was prepared according to the procedure described in Step 1 of Example 64 starting from intermediate 6.5 (139 mg, 0.48 mmol), HATU (218 mg, 0.57 mmol), DIPEA (0.25 mL, 1.44 mmol), 3.0 M EtMgBr in Et₂O (0.38 mL, 1.15 mmol), and intermediate 57.2 (115 mg, 0.57 mmol) in THF (2.0 mL+2.0 mL). Stirring was continued at r.t. for 16 h. After purification by flash chromatography (DCM/MeOH from 100% DCM to 9:1 v/v DCM/MeOH) the intermediate 168.1 (35 mg, 0.07 mmol) was obtained. Yield: 15%. MS-ESI(+) m/z: 474.3 (M+H); MS-ESI(−) m/z: 472.2 (M−H).

### Step 2: (±)-trans-4-Phenyl-N-[3-[(6-methylpyridin-3-yl)oxy]phenyl]pyrrolidine-3-carboxamide dihydrochloride (Compound I-105)

Compound I-104 was prepared following the procedure described in Step 2 of Example 120 starting from a solution of intermediate 168.1 (35 mg, 0.07 mmol) and a 0.9 M solution of HCl in EtOAc (0.8 mL). The title compound I-105 (31 mg, 0.0.07 mmol) was obtained as a white solid. Yield: quantitative. (400 MHz, DMSO-d₆) δ d 2.55 (s, 3H), 3.15-3.20 (m, 2H), 3.35-3.39 (m, 1H), 3.58-3.63 (m, 3H), 6.71-6.74 (m, 1H), 7.18-7.20 (m, 1H), 7.22-7.29 (m, 7H), 7.64 (m, 1H), 7.84 (d, J=8.7 Hz, 1H), 8.43 (s, 1H), 9.58 (brs, 1H), 9.94 (brs, 1H), 10.61 (s, 1H). HPLC purity: ≥95%. MS-ESI(+) m/z: 374.1 (M+H).

### Example 169: (±)-trans-4-Phenyl-N-[3-(pyridin-3-ylamino)phenyl]pyrrolidine-3-carboxamide dihydrochloride (Compound I-106)

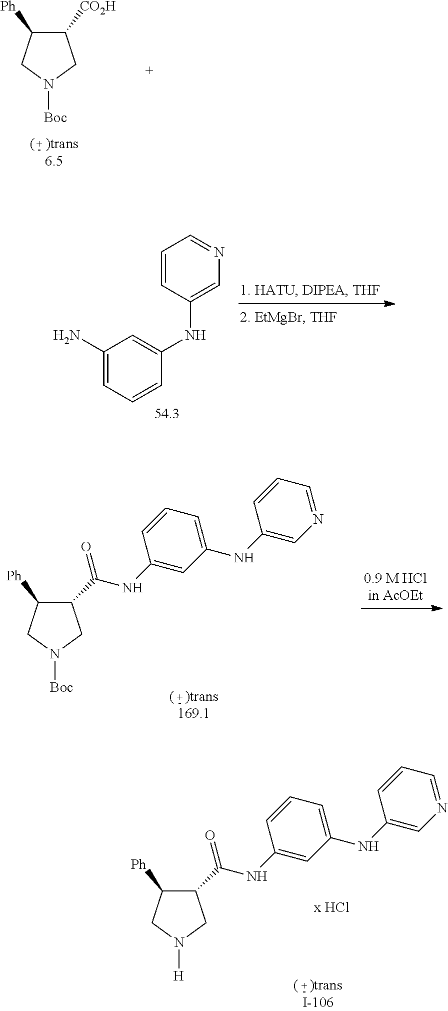

### Step 1: tert-Butyl (±)-trans-4-phenyl-N-[3-(pyridin-3-ylamino)phenyl]pyrrolidine-3-carboxamide-1-carboxylate (169.1)

Intermediate 169.1 was prepared according to the procedure described in Step 1 of Example 64 from intermediate 6.5 (236 mg, 0.81 mmol), intermediate 54.3 (150 mg, 0.81 mmol), EDC (233 mg, 1.21 mmol), HOBt (164 mg, 1.21 mmol), and DIPEA (0.56 mL, 3.24 mmol) in DCM (10 mL). Stirring was continued for 48 h. The intermediate 169.1 (108 mg, 0.24 mmol) was obtained after work-up and chromatographic purification (DCM/MeOH, from 100% DCM to 95:5 v/v DCM/MeOH). Yield: 29%. MS-ESI(+) m/z: 459.4 (M+H); MS-ESI(−) m/z: 457.4 (M−H).

### Step 2: (±)-trans-4-Phenyl-N-[3-(pyridin-3-ylamino)phenyl]pyrrolidine-3-carboxamide dihydrochloride (Compound I-106)

Compound I-106 was prepared following the procedure described in Step 2 of Example 120 starting from a solution of intermediate 169.1 (108 mg, 0.24 mmol) and a 0.9 M solution of HCl in EtOAc (2.1 mL). The title compound I-106 (87 mg, 0.20 mmol) was obtained as a yellow solid. Yield: 86%. (400 MHz, DMSO-d₆) δ 3.27-3.73 (m, 6H), 6.92 (dd, J=7.9 Hz, J2=1.3 Hz, 1H), 7.18 (d, J=8.1 Hz, 1H), 7.24-7.30 (m, 2H), 7.33-7.40 (m, 4H), 7.58 (m, 1H), 7.75 (dd, J1=8.7 Hz, J2=5.3 Hz, 1H), 7.97 (d, J=8.6 Hz, 1H), 8.21 (d, J=4.8 Hz, 1H), 8.38 (d, J=2.7 Hz, 1H), 9.34 (s, 1H), 9.50 (brs, 1H), 9.79 (brs, 1H), 10.45 (s, 1H). HPLC purity: ≥95%. MS-ESI(+) m/z: 359.1 (M+H).

### Example 170: (±)-trans-4-Phenyl-N-[4-(pyridin-3-ylamino)phenyl]pyrrolidine-3-carboxamide dihydrochloride (Compound I-107)

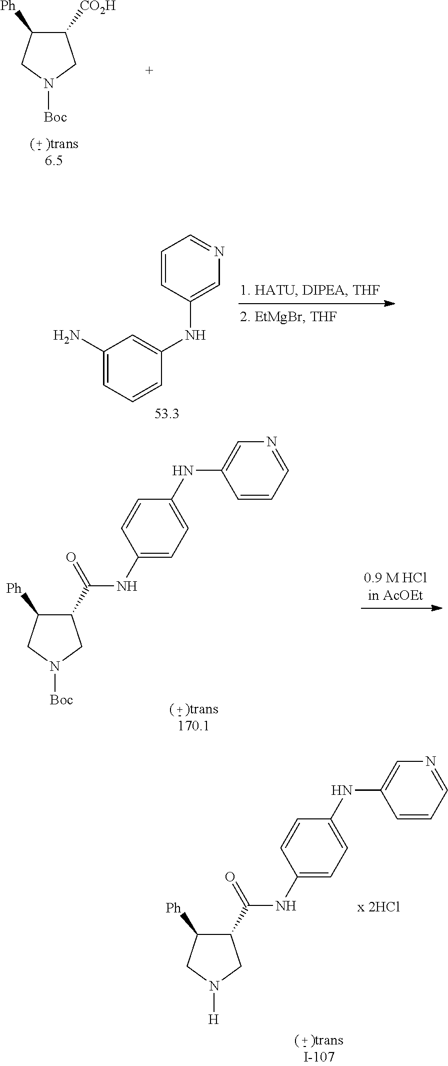

### Step 1: tert-Butyl (±)-trans-4-phenyl-N-[4-(pyridin-3-ylamino)phenyl]pyrrolidine-3-carboxamide-1-carboxylate (170.1)

Intermediate 170.1 was prepared according to the procedure described in Step 1 of Example 64 from intermediate 6.5 (228 mg, 0.73 mmol), intermediate 55.2 (145 mg, 0.78 mmol), EDC (225 mg, 1.17 mmol), HOBt (158 mg, 1.17 mmol), and DIPEA (0.55 mL, 3.13 mmol) in DCM (10 mL). Stirring was continued for 48 h. The intermediate 170.1 (47 mg, 0.10 mmol) was obtained after work-up and chromatographic purification (DCM/MeOH, from 100% DCM to 95:5 v/v DCM/MeOH). Yield: 14%. MS-ESI(+) m/z: 459.3 (M+H); MS-ESI(−) m/z: 457.1 (M−H).

### Step 2: (±)-trans-4-Phenyl-N-[4-(pyridin-3-ylamino)phenyl]pyrrolidine-3-carboxamide dihydrochloride (Compound I-107)

Compound I-107 was prepared following the procedure described in Step 2 of Example 120 starting from a solution of intermediate 170.1 (40 mg, 0.09 mmol) and a 0.9 M solution of HCl in EtOAc (1.0 mL). The title compound I-107 (23 mg, 0.06 mmol) was obtained as a yellow solid. Yield: 74%. (400 MHz, DMSO-d₆) δ 3.20-3.60 (m, 4H), 3.66-3.69 (m, 2H), 7.11 (d, J=8.9 Hz, 1H), 7.17-7.27 (m, 1H), 7.26-7.40 (m, 4H), 7.51 (d, J=8.9 Hz, 2H), 7.66 (dd, J₁=8.6 Hz, J₂=5.2 Hz, 1H), 7.85 (d, J=8.5 Hz, 1H), 8.10 (d, J=5.3 Hz, 1H), 8.24 (d, J=2.7 Hz, 1H), 9.20 (s, 1H), 9.40 (brs, 1H), 9.74 (brs, 1H), 10.33 (s, 1H). HPLC purity: ≥90%. MS-ESI(+) m/z: 359.1 (M+H).

### Example 171: (3S,4R)—N-[4-(6-Fluoropyridin-3-yl)phenyl]-4-phenylpyrrolidine-3-carboxamide dihydrochloride (Compound I-108)

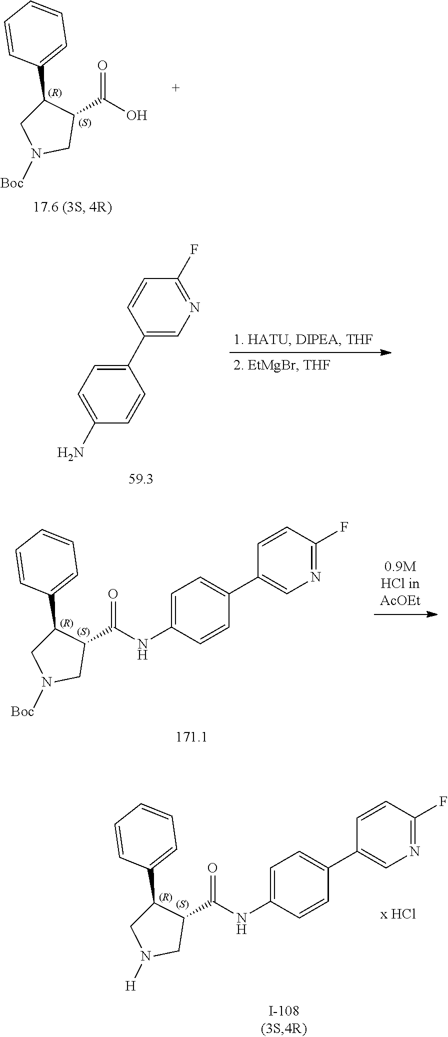

### Step 1: tert-Butyl (3S,4R)-4-phenyl-N-[4′-fluorobiphenyl-4-yl]pyrrolidine-3-carboxamide-1-carboxylate (171.1)

Intermediate 171.1 was prepared according to the procedure described in Step 1 of Example 64 starting from a solution of intermediate 17.6 (250 mg, 0.86 mmol), HATU (424.1 mg, 1.11 mmol), DIPEA (0.45 mL, 2.58 mmol), 3.0 M EtMgBr in Et₂O (0.31 mL, 0.94 mmol), and intermediate 59.3 (177 mg, 0.94 mmol) in THF (5 mL+5 mL). Stirring was continued at r.t. for 16 h. After purification by flash chromatography (PET/EtOAc, from 100% PET to 70:30 v/v PET/EtOAc) the intermediate 171.1 (110 mg, 0.27 mmol) was obtained as a colorless oil. Yield 31%. MS-ESI(+) m/z: 462.1 (M+H).

### Step 2: (3S,4R)—N-[4-(6-Fluoropyridin-3-yl)phenyl]-4-phenylpyrrolidine-3-carboxamide dihydrochloride (Compound I-108)

Compound I-108 was prepared following the procedure described in Step 2 of Example 120 starting from a solution of intermediate 170.1 (100 mg, 0.22 mmol) and a 0.9 M solution of HCl in EtOAc (2.7 mL). The title compound I-108 (50 mg, 0.11 mmol) was obtained as a white solid. Yield: 50%. ¹H NMR (400 MHz, DMSO-d₆) δ 3.30-3.35 (m, 2H), 3.34-3.46 (m, 1H), 3.74 (m, 4H), 7.24-7.26 (m, 2H), 7.36-7.39 (m, 4H), 7.60-7.67 (m, 4H), 8.23 (m, 1H), 8.50 (s, 1H), 9.52 (brs, 1H), 9.89 (brs, 1H), 10.52 (s, 1H). HPLC purity: ≥90%. MS-ESI(+) m/z: 362.1 (M+H).

### Example 172: (3R,4S)—N-(Naphthalen-1-yl)-4-[4-(trifluoromethyl)phenyl]pyrrolidine-3-carboxamide hydrochloride (Compound I-109)

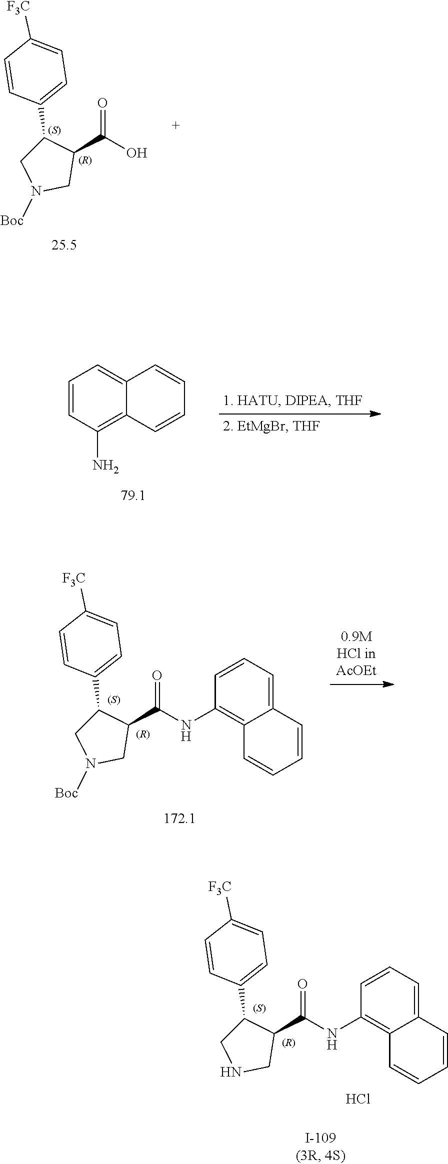

### Step 1: tert-Butyl (3R,4S)—N-(naphthalen-1-yl)-4-[4-(trifluoromethyl)phenyl]pyrrolidine-3-carboxamide-1-carboxylate (172.1)

Intermediate 172.1 was prepared according to the procedure described in Step 1 of Example 64 starting from a solution of intermediate 18.3 (230 mg, 0.64 mmol), HATU (316.4 mg, 0.83 mmol), DIPEA (0.33 mL, 1.92 mmol), 3.0 M EtMgBr in Et₂O (0.43 mL, 0.43 mmol), and intermediate 79.1 (110 mg, 0.77 mmol) in THF (5 mL+5 mL). Stirring was continued at r.t. for 16 h. After purification by flash chromatography (PET/EtOAc, from 100% PET to 80:20 v/v PET/EtOAc) the intermediate 172.1 (54 mg, 0.11 mmol) was obtained as a colorless oil. Yield 17%. MS-ESI(+) m/z: 485.1 (M+H).

### Step 2: (3R,4S)—N-(Naphthalen-1-yl)-4-[4-(trifluoromethyl)phenyl]pyrrolidine-3-carboxamide hydrochloride (Compound I-109)

Compound I-109 was prepared following the procedure described in Step 2 of Example 120 starting from a solution of intermediate 172.1 (54 mg, 0.11 mmol) and a 0.9 M solution of HCl in EtOAc (1.4 mL). The title compound I-109 (35 mg, 0.0.83 mmol) was obtained as a grey solid. Yield: 76%. ¹H NMR (400 MHz, DMSO-d₆) δ 3.38-3.50 (m, 2H), 3.68-3.74 (m, 1H), 3.77-3.87 (m, 3H), 7.31-7.37 (m, 2H), 7.75 (d, J=8.1 Hz, 2H), 7.48-7.54 (m, 2H), 7.57 (d, J=7.3 Hz, 1H), 7.79 (d, J=8.1 Hz, 1H), 7.84 (d, J=8.2 Hz, 2H), 7.93 (d, J=8.3 Hz, 1H), 9.66 (brs, 1H), 10.01 (brs, 1H), 10.22 (s, 1H). HPLC purity: >95%. HPLC purity: ≥95%. MS-ESI(+) m/z: 385.2 (M+H).

### Example 173: (3S,4R)—N-(Naphthalen-1-yl)-4-[4-(trifluoromethyl)phenyl]pyrrolidine-3-carboxamide hydrochloride (Compound I-110)

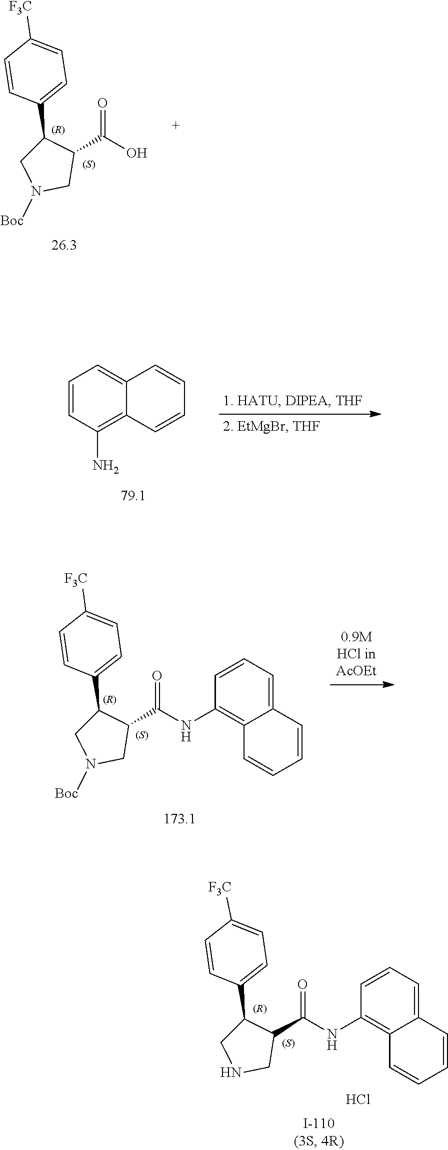

### Step 1: tert-Butyl (3S,4R)—N-(naphthalen-1-yl)-4-[4-(trifluoromethyl)phenyl]pyrrolidine-3-carboxamide-1-carboxylate (173.1)

Intermediate 173.1 was prepared according to the procedure described in Step 1 of Example 64 starting from intermediate 26.3 (250 mg, 0.70 mmol), HATU (317 mg, 0.83 mmol), DIPEA (0.36 pmL, 2.09 mmol), 3.0 M EtMgBr in Et₂O (0.70 mL, 2.09 mmol), and intermediate 79.1 (149 mg, 1.04 mmol) in THF (5.0 mL+5.0 mL). Stirring was continued at r.t. for 16 h. After purification by flash chromatography (DCM/MeOH from 100% DCM to 9:1 v/v DCM/MeOH) the intermediate 173.1 (84 mg, 0.17 mmol) was obtained. Yield: 25%. MS-ESI(−) m/z: 482.9 (M−H).

### Step 2: (3S,4R)—N-(Naphthalen-1-yl)-4-[4-(trifluoromethyl)phenyl]pyrrolidine-3-carboxamide hydrochloride (Compound I-110)

Compound I-110 was prepared following the procedure described in Step 2 of Example 120 starting from a solution of intermediate 173.1 (84 mg, 0.17 mmol) and a 0.9 M solution of HCl in EtOAc (2.0 mL). The title compound I-110 (69 mg, 0.16 mmol) was obtained as a pale grey solid. Yield: 96%. (400 MHz, DMSO-d₆) δ 3.36-3.44 (m, 2H), 3.58-3.62 (m, 1H), 3.70-3.79 (m, 3H), 7.26-7.27 (m, 2H), 7.41-7.50 (m, 3H), 7.66 (d, J=8.2 Hz, 2H), 7.71 (d, J=8.1 Hz, 1H), 7.76 (d, J=8.2 Hz, 2H), 7.84 (d, J=7.9 Hz, 1H), 9.41 (brs, 1H), 9.68 (brs, 1H), 10.08 (s, 1H). HPLC purity: ≥95%. MS-ESI(+) m/z: 385.2 (M+H).

### Example 174: (3R,4S)—N-[3-(4-Cyanophenoxy)phenyl]-4-phenylpyrrolidine-3-carboxamide hydrochloride (Compound I-111)

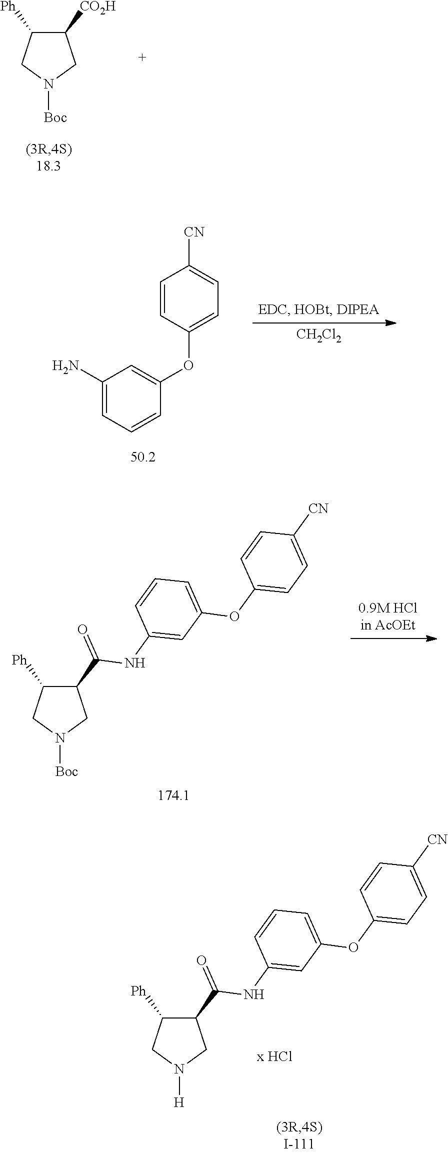 [SMILES: CC(C)(C)OC(=O)N1C[C@H](C(=O)O)[C@@H](c2ccccc2)C1 ; N#Cc1ccc(Oc2cccc(N)c2)cc1 ; "CH2Cl2" ; CC(C)(C)OC(=O)N1C[C@H](C(=O)Nc2cccc(Oc3ccc(C#N)cc3)c2)[C@@H](c2ccccc2)C1 ; N#Cc1ccc(Oc2cccc(NC(=O)[C@H]3CNC[C@@H]3c3ccccc3)c2)cc1]

### Step 1: tert-Butyl (3R,4S)—N-[3-(4-cyanophenoxy)phenyl]-4-phenylpyrrolidine-3-carboxamide-1-carboxylate (174.1)

Intermediate 174.1 was prepared according to the procedure described in Step 1 of Example 94 from intermediate 18.3 (150 mg, 0.51 mmol), intermediate 50.2 (108 mg, 0.51 mmol), EDC (148 mg, 0.77 mmol), HOBt (104 mg, 0.77 mmol), and DIPEA (0.36 mL, 2.06 mmol) in DCM (5 mL). Stirring was continued for 48 h. The intermediate 174.1 (34 mg, 0.07 mmol) was obtained after work-up and chromatographic purification (PET/EtOAc, from 100% PET to 1:1 v/v PET/EtOAc) as a yellow solid. Yield: 14%. MS-ESI(−) m/z: 482.3 (M−H).

### Step 2: (3R,4S)—N-[3-(4-Cyanophenoxy)phenyl]-4-phenylpyrrolidine-3-carboxamide hydrochloride (Compound I-111)

Compound I-111 was prepared following the procedure described in Step 2 of Example 120 starting from a solution of intermediate 174.1 (34 mg, 0.07 mmol) and a 0.9 M solution of HCl in EtOAc (0.6 mL). The title compound I-111 (21 mg, 0.05 mmol) was obtained as a pale yellow solid. Yield: 72%. (400 MHz, DMSO-d₆) δ 3.18-3.30 (m, 3H), 3.59-3.65 (m, 3H), 6.74-6.76 (m, 1H), 7.01 (d, J=9.0 Hz, 2H), 7.19-7.33 (m, 8H), 7.76 (d, J=8.9 Hz, 2H), 9.23 (brs, 1H), 9.53 (brs, 1H), 10.38 (s, 1H). HPLC purity: ≥90%. MS-ESI(+) m/z: 384.2 (M+H).

### Example 175: (3S,4R)—N-[3-(4-Cyanophenoxy)phenyl]-4-phenylpyrrolidine-3-carboxamide hydrochloride (Compound I-112)

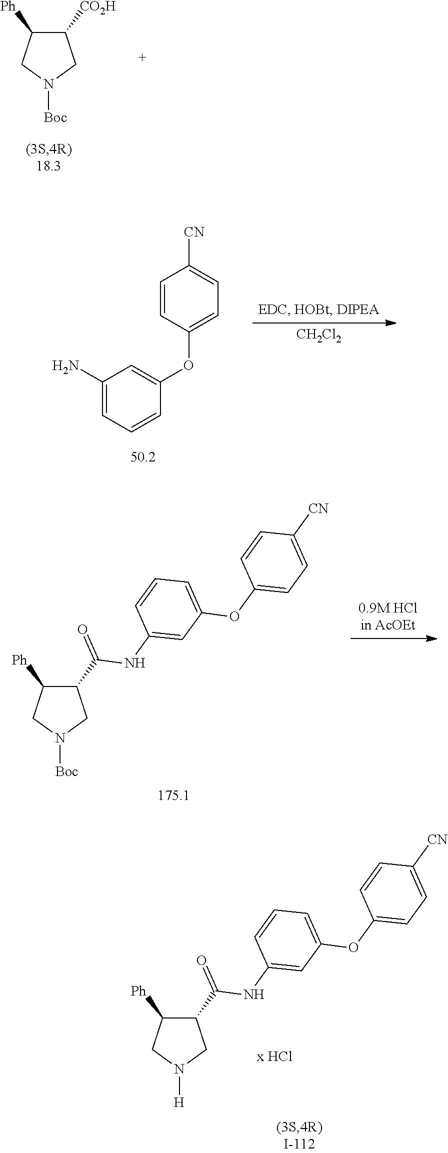 [SMILES: CC(C)(C)OC(=O)N1C[C@@H](C(=O)O)[C@H](c2ccccc2)C1 ; N#Cc1ccc(Oc2cccc(N)c2)cc1 ; "CH2Cl2" ; CC(C)(C)OC(=O)N1C[C@@H](C(=O)Nc2cccc(Oc3ccc(C#N)cc3)c2)[C@H](c2ccccc2)C1 ; N#Cc1ccc(Oc2cccc(NC(=O)[C@@H]3CNC[C@H]3c3ccccc3)c2)cc1]

### Step 1: tert-Butyl (3S,4R)—N-[3-(4-cyanophenoxy)phenyl]-4-phenylpyrrolidine-3-carboxamide-1-carboxylate (175.1)

Intermediate 175.1 was prepared according to the procedure described in Step 1 of Example 94 from intermediate 18.3 (150 mg, 0.51 mmol), intermediate 50.2 (108 mg, 0.51 mmol), EDC (148 mg, 0.77 mmol), HOBt (104 mg, 0.77 mmol), and DIPEA (0.36 mL, 2.06 mmol) in DCM (5 mL). Stirring was continued for 48 h. The intermediate 175.1 (47 mg, 0.10 mmol) was obtained after work-up and chromatographic purification (PET/EtOAc, from 100% PET to 1:1 v/v PET/EtOAc) as a yellow solid. Yield: 19%. MS-ESI(−) m/z: 482.4 (M−H).

### Step 2: (3S,4R)—N-[3-(4-Cyanophenoxy)phenyl]-4-phenylpyrrolidine-3-carboxamide hydrochloride (Compound I-112)

Compound I-112 was prepared following the procedure described in Step 2 of Example 120 starting from a solution of intermediate 175.1 (47 mg, 0.10 mmol) and a 0.9 M solution of HCl in EtOAc (0.9 mL). The title compound I-112 (36 mg, 0.09 mmol) was obtained as a pale yellow solid. Yield: 88%. (400 MHz, DMSO-d₆) δ 3.21-3.33 (m, 3H), 3.66-3.72 (m, 3H), 6.83-6.84 (m, 1H), 7.10 (d, J=8.80 Hz, 2H), 7.22-7.45 (m, 8H), 7.85 (d, J=8.8 Hz, 2H), 9.33 (brs, 1H), 9.63 (brs, 1H), 10.48 (s, 1H). HPLC purity: ≥90%. MS-ESI(+) m/z: 384.2 (M+H).

### Example 176: (3R,4S)-4-Phenyl-N-[3-(phenylamino)phenyl]pyrrolidine-3-carboxamide hydrochloride (Compound I-113)

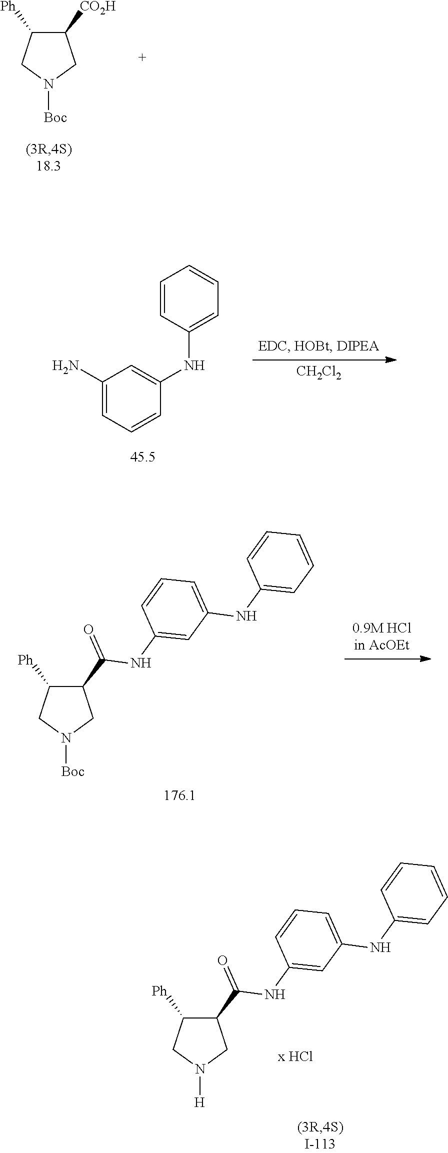 [SMILES: CC(C)(C)OC(=O)N1C[C@H](C(=O)O)[C@@H](c2ccccc2)C1 ; Nc1cccc(Nc2ccccc2)c1 ; "CH2Cl2" ; CC(C)(C)OC(=O)N1C[C@H](C(=O)Nc2cccc(Nc3ccccc3)c2)[C@@H](c2ccccc2)C1 ; O=C(Nc1cccc(Nc2ccccc2)c1)[C@H]1CNC[C@@H]1c1ccccc1]

### Step 1: tert-Butyl (3R,4S)-4-phenyl-N-[3-(phenylamino)phenyl]pyrrolidine-3-carboxamide-1-carboxylate (176.1)

Intermediate 176.1 was prepared according to the procedure described in Step 1 of Example 94 from intermediate 18.5 (277 mg, 0.95 mmol), intermediate 45.5 (175 mg, 0.95 mmol), EDC (273 mg, 1.42 mmol), HOBt (193 mg, 1.42 mmol), and DIPEA (0.66 mL, 3.80 mmol) in DCM (10 mL). Stirring was continued for 3 days. The intermediate 176.1 (141 mg, 0.31 mmol) was obtained after work-up and chromatographic purification (PET/EtOAc, from 100% PET to 1:1 v/v PET/EtOAc) as a pale yellow oil. Yield: 32%. MS-ESI(−) m/z: 456.1 (M−H).

### Step 2: (3R,4S)-4-Phenyl-N-[3-(phenylamino)phenyl]pyrrolidine-3-carboxamide hydrochloride (Compound I-113)

Compound I-113 was prepared following the procedure described in Step 2 of Example 120 starting from a solution of intermediate 176.1 (100 mg, 0.22 mmol) and a 0.9 M solution of HCl in EtOAc (2.4 mL). The title compound I-113 (86 mg, 0.22 mmol) was obtained as a white solid. Yield: quantitative. (400 MHz, DMSO-d₆) δ 3.23-3.43 (m, 3H), 3.68-3.75 (m, 3H), 6.72 (dd, J1=8.0 Hz, J2=1.3 Hz, 1H), 6.83 (t, J=7.3 Hz, 1H), 6.98 (d, J=8.0 Hz, 1H), 7.04-7.12 (m, 3H), 7.21-7.30 (m, 3H), 7.34-7.41 (m, 5H), 9.48 (brs, 1H), 9.88 (brs, 1H), 10.17 (s, 1H). HPLC purity: ≥95%. MS-ESI(+) m/z: 358.2 (M+H).

### Example 177: (3S,4R)-4-Phenyl-N-[3-(phenylamino)phenyl]pyrrolidine-3-carboxamide hydrochloride (Compound I-114)

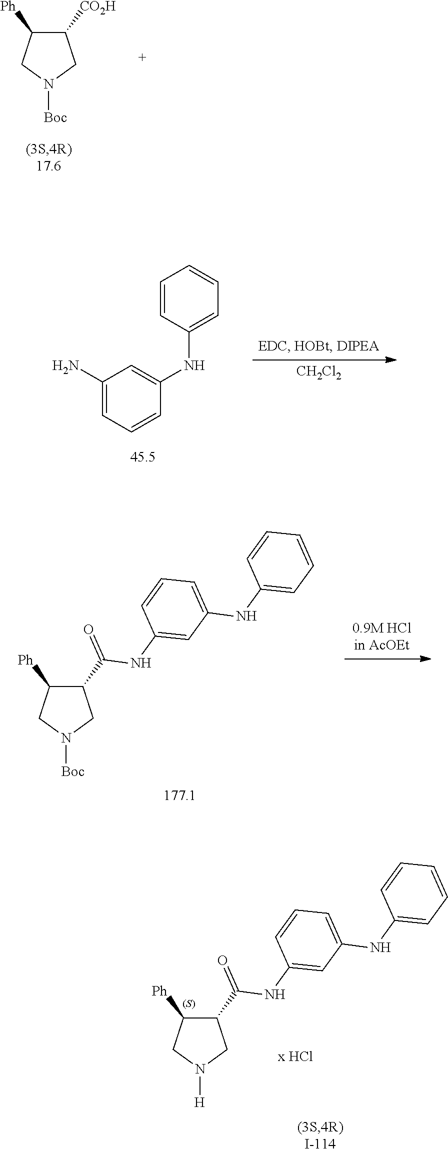

### Step 1: tert-Butyl (3S,4R)-4-phenyl-N-[3-(phenylamino)phenyl]pyrrolidine-3-carboxamide-1-carboxylate (177.1)

Intermediate 177.1 was prepared according to the procedure described in Step 1 of Example 94 from intermediate 17.6 (277 mg, 0.95 mmol), intermediate 45.5 (175 mg, 0.95 mmol), EDC (273 mg, 1.42 mmol), HOBt (193 mg, 1.42 mmol), and DIPEA (0.66 mL, 3.80 mmol) in DCM (10 mL). Stirring was continued for 3 days. The intermediate 177.1 (108 mg, 0.24 mmol) was obtained after work-up and chromatographic purification (PET/EtOAc, from 100% PET to 1:1 v/v PET/EtOAc) as a pale yellow oil. Yield: 25%. MS-ESI(−) m/z: 456.1 (M−H).

### Step 2: (3S,4R)-4-Phenyl-N-[3-(phenylamino)phenyl]pyrrolidine-3-carboxamide hydrochloride (Compound I-114)

Compound I-114 was prepared following the procedure described in Step 2 of Example 120 starting from a solution of intermediate 177.1 (100 mg, 0.22 mmol) and a 0.9 M solution of HCl in EtOAc (2.3 mL). The title compound I-114 (84 mg, 0.21 mmol) was obtained as a white solid. Yield: 97%. (400 MHz, DMSO-d₆) δ 3.23-3.43 (m, 3H), 3.68-3.75 (m, 3H), 6.72 (d, J=8.2 Hz, 1H), 6.82 (t, J=7.0 Hz, 1H), 6.96 (d, J=8.1 Hz, 1H), 7.03-7.11 (m, 3H), 7.20-7.29 (m, 3H), 7.32-7.44 (m, 5H), 9.55 (brs, 1H), 9.91 (brs, 1H), 10.20 (s, 1H).

HPLC purity: ≥95%. MS-ESI(+) m/z: 358.2 (M+H).

### Example 178: (±)-trans-N-[trans-3-(4-Methylphenoxy)cyclobutyl]-4-phenylpyrrolidine-3-carboxamide hydrochloride (Compound I-115)

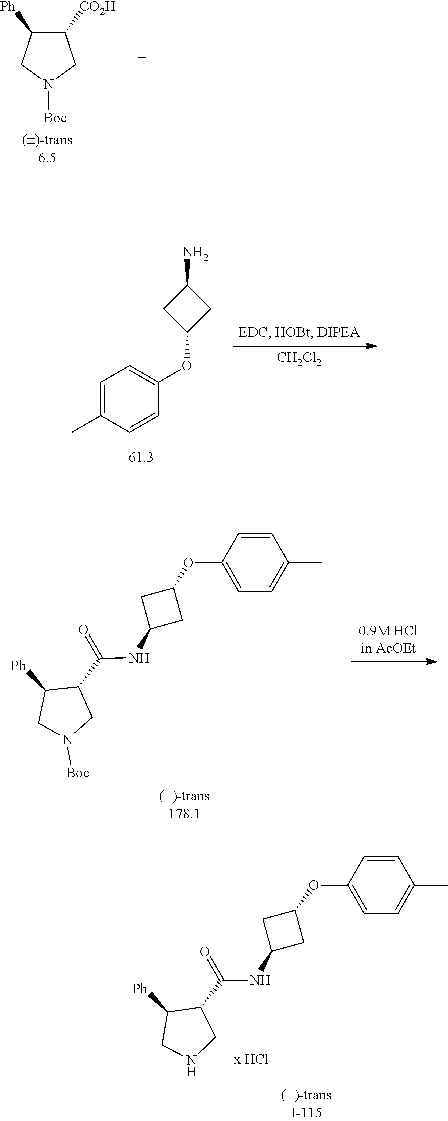 [SMILES: CC(C)(C)OC(=O)N1C[C@@H](C(=O)O)[C@H](c2ccccc2)C1 ; Cc1ccc(O[C@H]2C[C@H](N)C2)cc1 ; "CH2Cl2" ; Cc1ccc(O[C@H]2C[C@H](NC(=O)[C@@H]3CN(C(=O)OC(C)(C)C)C[C@H]3c3ccccc3)C2)cc1 ; Cc1ccc(O[C@H]2C[C@H](NC(=O)[C@@H]3CNC[C@H]3c3ccccc3)C2)cc1]

### Step 1: tert-Butyl (±)-trans-4-phenyl-N-[trans-3-(4-methylphenoxy)cyclobutyl]pyrrolidine-3-carboxamide-1-carboxylate (178.1)

Intermediate 178.1 was prepared according to the procedure described in Step 1 of Example 94 starting from a solution of intermediate 6.5 (150 mg, 0.51 mmol), EDC (148 mg, 0.77 mmol), HOBt (104 mg, 0.77 mmol), DIPEA (0.36 μL, 2.04 mmol), and intermediate 61.3 (119 mg, 0.56 mmol) in DCM (10 mL). The intermediate 178.1 (200 mg, 0.44 mmol) was obtained after work-up and chromatographic purification (PET/EtOAc, from 100% PET to 65:35 v/v PET/EtOAc) as a colorless oil. Yield: 87%. ESI(+) m/z: 451.2 (M+H).

### Step 2: (±)-trans-N-[trans-3-(4-Methylphenoxy)cyclobutyl]-4-phenylpyrrolidine-3-carboxamide hydrochloride (Compound I-115)

Compound I-115 was prepared following the procedure described in Step 2 of Example 120 starting from a solution of intermediate 178.1 (180 mg, 0.39 mmol) and a 0.9 M solution of HCl in EtOAc (4 mL). The title Compound I-115 (100 mg, 0.26 mmol) was obtained as a white solid. Yield: 67%. ¹H NMR (400 MHz, DMSO-d₆) δ 2.08-2.15 (m, 1H), 2.24 (s, 3H), 2.28-2.32 (m, 2H), 3.09-3.16 (m, 1H), 3.25-3.30 (m, 2H), 3.37 (brs, 2H), 3.57-3.71 (m, 3H), 4.15-4.25 (m, 1H), 4.62-4.68 (m, 1H), 6.66 (d, J=8.4 Hz, 2H), 7.08 (d, J=8 Hz, 2H), 7.30-7.42 (m, 4H), 8.63 (d, J=6.8 Hz, 1H), 9.57 (brs, 2H). HPLC purity: ≥95%. MS-ESI(+) m/z: 351.3 (M+H).

### Example 179: (±)-trans-N-{trans-3-[(6-Fluoropyridin-3-yl)oxy]cyclobutyl}-4-phenylpyrrolidine-3-carboxamide dihydrochloride (Compound I-116)

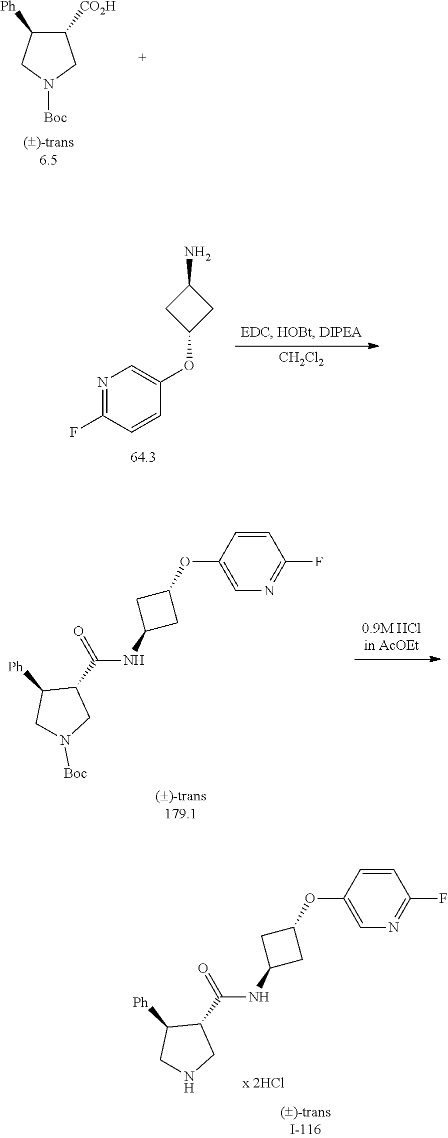 [SMILES: CC(C)(C)OC(=O)N1C[C@@H](C(=O)O)[C@H](c2ccccc2)C1 ; N[C@H]1C[C@H](Oc2ccc(F)nc2)C1 ; "CH2Cl2" ; CC(C)(C)OC(=O)N1C[C@@H](C(=O)N[C@H]2C[C@H](Oc3ccc(F)nc3)C2)[C@H](c2ccccc2)C1 ; O=C(N[C@H]1C[C@H](Oc2ccc(F)nc2)C1)[C@@H]1CNC[C@H]1c1ccccc1]

### Step 1: tert-Butyl-(±)-trans-N-[trans-3-[(6-fluoropyridin-3-yl)oxy]cyclobutyl]-4-phenylpyrrolidine-3-carboxamide-1-carboxylate (179.1)

Intermediate 179.1 was prepared according to the procedure described in Step 1 of Example 94 starting from a solution of intermediate 6.5 (160 mg, 0.55 mmol), EDC (158 mg, 0.82 mmol), HOBt (111 mg, 0.82 mmol), DIPEA (0.38 mL, 2.2 mmol), and intermediate 64.3 (154 mg, 0.6 mmol) in DCM (10 mL). The intermediate 179.1 (180 mg, 0.39 mmol) was obtained after work-up and chromatographic purification (PET/EtOAc, from 100% PET to 30:70 v/v PET/EtOAc) as a colorless oil. Yield: 72%. ESI(+) m/z: 456.1 (M+H).

### Step 2: (±)-trans-N-[trans-3-[(6-Fluoropyridin-3-yl)oxy]cyclobutyl]-4-phenylpyrrolidine-3-carboxamide hydrochloride (Compound I-116)

Compound I-116 was prepared following the procedure described in Step 3 of Example 96 starting from a solution of intermediate 179.1 (180 mg, 0.39 mmol) and a 0.9 M solution of HCl in EtOAc (2.4 mL). The title Compound I-116 (100 mg, 0.26 mmol) was obtained as a white solid. Yield: 95%. ¹H NMR (400 MHz, DMSO-d₆) δ 2.08-2.15 (m, 1H), 2.28-2.35 (m, 3H), 3.06-3.13 (m, 1H), 3.22-3.27 (m, 2H), 3.34 (brs, 3H), 3.54-3.69 (m, 3H), 4.18-4.27 (m, 1H), 4.71-4.77 (m, 1H), 7.12 (dd, J=8.9 Hz, J=3.4 Hz, 1H), 7.27-7.38 (m, 5H), 7.41-7.45 (m, 1H), 7.72 (dd, J=2.9, 2.0 Hz, 1H), 8.63 (d, J=6.8 Hz, 1H), 9.55 (brs, 2H). HPLC purity: ≥95%. MS-ESI(+) m/z: 356.2 (M+H).

### Example 180: (3S,4R)-4-Phenyl-N-(trans-3-phenylcyclobutyl)pyrrolidine-3-carboxamide hydrochloride (Compound I-117)

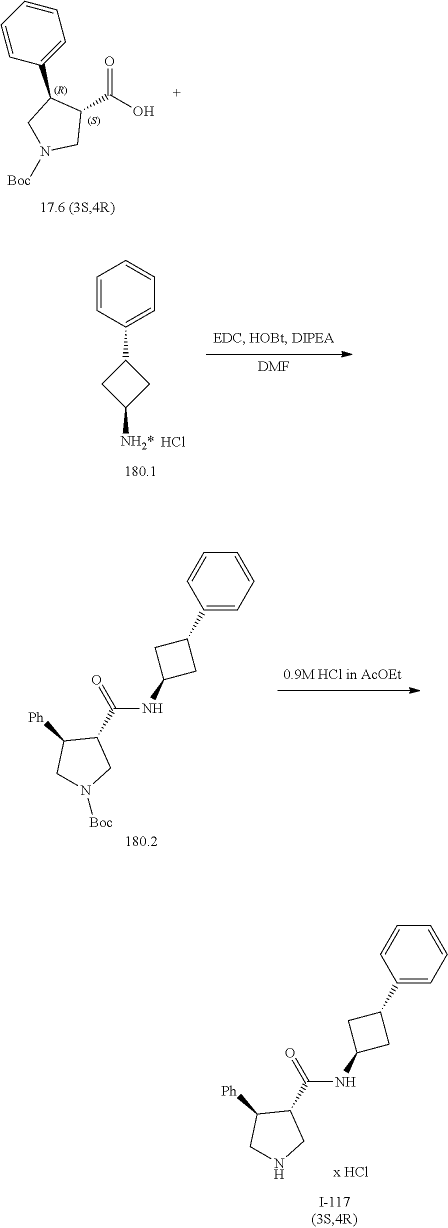

### Step 1: tert-Butyl-(3S,4R)-3-[(trans-3-phenylcyclobutyl)carbamoyl]-4-phenylpyrrolidine-1-carboxylate (180.2)

Intermediate 180.2 was prepared according to the procedure described in Step 1 of Example 94 starting from a solution of intermediate 17.6 (200 mg, 0.69 mmol), EDC (145 mg, 0.76 mmol), HOBt (98 mg, 0.72 mmol), DIPEA (0.47 mL, 2.75 mmol), and intermediate 180.1 (132 mg, 0.72 mmol) in DMF (4 mL). The intermediate 180.2 (111 mg, 0.26 mmol) was obtained after work-up and chromatographic purification (PET/EtOAc, from 100% PET to 40:60 v/v PET/EtOAc) as a yellowish sticky oil. Yield: 38%. ESI(+) m/z: 421.2 (M+H); MS-ESI(−) m/z: 419.2 (M−H).

### Step 2: (3S,4R)-4-Phenyl-N-(trans-3-phenylcyclobutyl)pyrrolidine-3-carboxamide hydrochloride (Compound I-117)

Compound I-117 was prepared following the procedure described in Step 2 of Example 120 starting from a solution of intermediate 180.2 (111 mg, 0.26 mmol) and a 0.9 M solution of HCl in EtOAc (2.9 mL). The title Compound I-117 (40 mg, 0.11 mmol) was obtained as a white solid. Yield: 42%. ¹H NMR (400 MHz, DMSO-d₆) δ 2.11-2.15 (m, 1H), 2.25-2.33 (m, 3H), 3.11-3.18 (m, 1H), 3.25-3.28 (m, 2H), 3.41-3.44 (m, 1H), 3.56-3.67 (m, 3H), 4.17-4.22 (m, 1H), 7.15-7.21 (m, 1H), 7.22-7.25 (m, 2H), 7.28-7.32 (m, 3H), 7.33-7.40 (m, 1H), 8.64 (d, J=5.8 Hz, 1H), 9.44 (brs, 1H), 9.79 (brs, 1H). HPLC purity: ≥90%. ESI(+) m/z: 321.2 (M+H); MS-ESI(−) m/z: 319.1 (M−H).

### Example 181: (3S,4R)-4-Phenyl-N-(1-phenylazetidin-3-yl)pyrrolidine-3-carboxamide hydrochloride (Compound I-118)

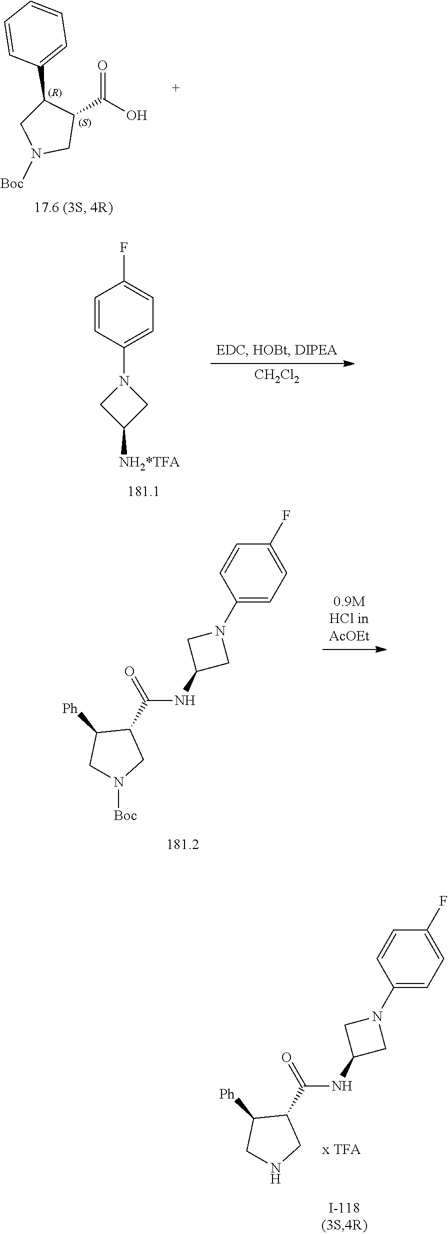

### Step 1: tert-Butyl-(3S,4R)-3-[(1-4fluorophenylazetidin-3-yl)carbamoyl]-4-phenylpyrrolidine-1-carboxylate (181.2)

Intermediate 181.2 was prepared according to the procedure described in Step of Example 94 starting from a solution of intermediate 17.6 (170 mg, 0.58 mmol), EDC (166 mg, 0.87 mmol), HOBt (117 mg, 0.87 mmol), DIPEA (0.4 mL, 2.32 mmol), and intermediate 181.1 (130 mg, 0.64 mmol) in DCM (15 mL). The intermediate 181.2 (40 mg, 0.09 mmol) was obtained after work-up and chromatographic purification (PET/EtOAc, from 100% PET to 30:70 v/v PET/EtOAc) as a colorless oil. Yield: 15%. ESI(−) m/z: 438.2 (M−H).

### Step 2: (3S,4R)-4-Phenyl-N-(1-4-fluorophenylazetidin-3-yl)pyrrolidine-3-carboxamide hydrochloride (Compound I-118)

To a solution of intermediate 181.2 (40 mg, 0.09 mmol) in DCM (2 mL) was added TFA (0.035 mL, 0.45 mmol) and the solution was stirred at r.t. 16 h. The title Compound I-118 (30 mg, 0.068 mmol) was obtained as a red oil. Yield: 75%. ¹H NMR (400 MHz, DMSO-de) δ 3.10 (d, J=4 Hz, 1H), 3.17 (d, J=4 Hz, 1H), 3.27 (m, 1H), 3.34-3.54 (m, 3H), 3.62-3.76 (m, 3H), 4.20 (d, J=4 Hz, 1H); 6.53-6.59 (m, 2H), 6.94 (t, J=8 Hz, 2H), 7.21-7.40 (m, 6H), 8.15 (brs, 3H), 9.33-9.46 (brs, 1H), 9.46 (brs, 1H). HPLC purity: ≥90%. MS-ESI(+) m/z: 340.3 (M+H).

### Example 182: 3R,4S)-4-Phenyl-N-(3-{[6-(trifluoromethyl)pyridin-3-yl]oxy}phenyl) pyrrolidine-3-carboxamide dihydrochloride (Compound I-119)

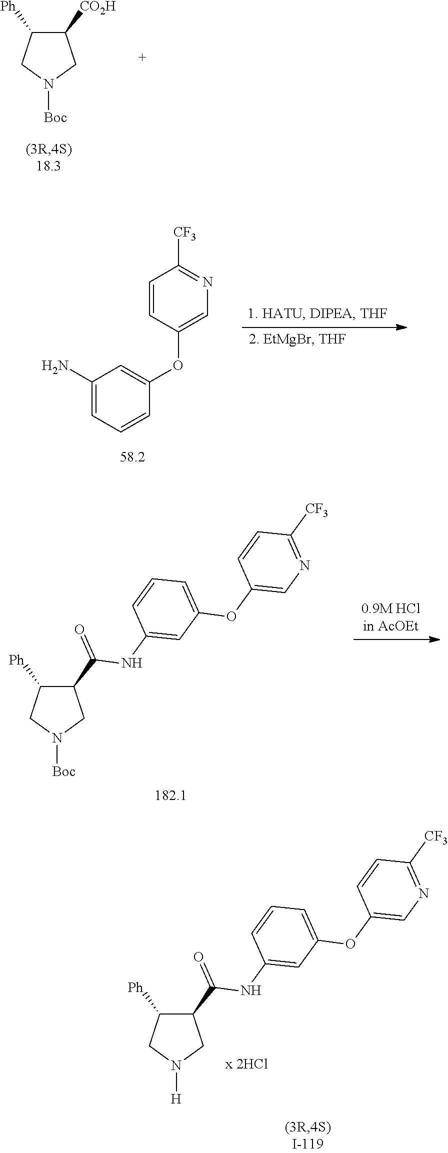 [SMILES: CC(C)(C)OC(=O)N1C[C@H](C(=O)O)[C@@H](c2ccccc2)C1 ; Nc1cccc(Oc2ccc(C(F)(F)F)nc2)c1 ; "2. EtMgBr, THF" ; CC(C)(C)OC(=O)N1C[C@H](C(=O)Nc2cccc(Oc3ccc(C(F)(F)F)nc3)c2)[C@@H](c2ccccc2)C1 ; O=C(Nc1cccc(Oc2ccc(C(F)(F)F)nc2)c1)[C@H]1CNC[C@@H]1c1ccccc1]

### Step 1: tert-Butyl-(3R,4S)-3-{[(6-trifluoromethylpyridin-3-yl)oxy]phenyl}carbamoyl]-4-phenylpyrrolidine-1-carboxylate (182.1)

Intermediate 182.1 was prepared according to the procedure described in Step 1 of Example 64 starting from intermediate 18.3 (210 mg, 0.72 mmol), HATU (356.3 mg, 0.93 mmol), DIPEA (0.38 mL, 2.16 mmol), 3.0 M EtMgBr in Et₂O (0.24 mL, 0.72 mmol), and intermediate 58.2 (200 mg, 0.79 mmol) in THF (5.0 mL+5.0 mL). Stirring was continued at r.t. for 16 h. After purification by flash chromatography (PET/EtOAc from 100% PET to 65:35 v/v PET/EtOAc) the intermediate 182.1 (180 mg, 0.34 mmol) was obtained. Yield: 47%. MS-ESI(−) m/z: 526.1 (M−H).

### Step 2: 3R, 4S)-4-Phenyl-N-(3-{[6-(trifluoromethyl)pyridin-3-yl]oxy}phenyl) pyrrolidine-3-carboxamide dihydrochloride(Compound I-119)

Compound I-119 was prepared following the procedure described in Step 2 of Example 120 starting from a solution of intermediate 182.1 (180 mg, 0.34 mmol) and a 0.9 M solution of HCl in EtOAc (1.7 mL). The title Compound I-119 (100 mg, 0.2 mmol) was obtained as a pale grey solid. Yield: 59%. (400 MHz, DMSO-d₆) δ 3.21-3.35 (m, 2H), 3.37-3.44 (m, 1H), 3.66-3.72 (m, 3H), 6.87 (dt, J=8 Hz, J=4 Hz, 1H), 7.24-7.28 (m, 1H), 7.31-7.40 (m, 6H), 7.45 (m, 1H), 7.55 (dd, J=12 Hz, J=4 Hz, 1H), 7.89 (d, J=8 Hz, 1H), 8.53 (d, J=4 Hz, 1H), 9.45 (brs, 1H), 9.58 (brs, 1H), 10.56 (s, 1H). HPLC purity: ≥95%. MS-ESI(+) m/z: 428.1 (M+H).

### Example 183: (3S,4R)-4-Phenyl-N-(3-{[6-(trifluoromethyl)pyridin-3-yl]oxy}phenyl) pyrrolidine-3-carboxamide dihydrochloride (Compound I-120)

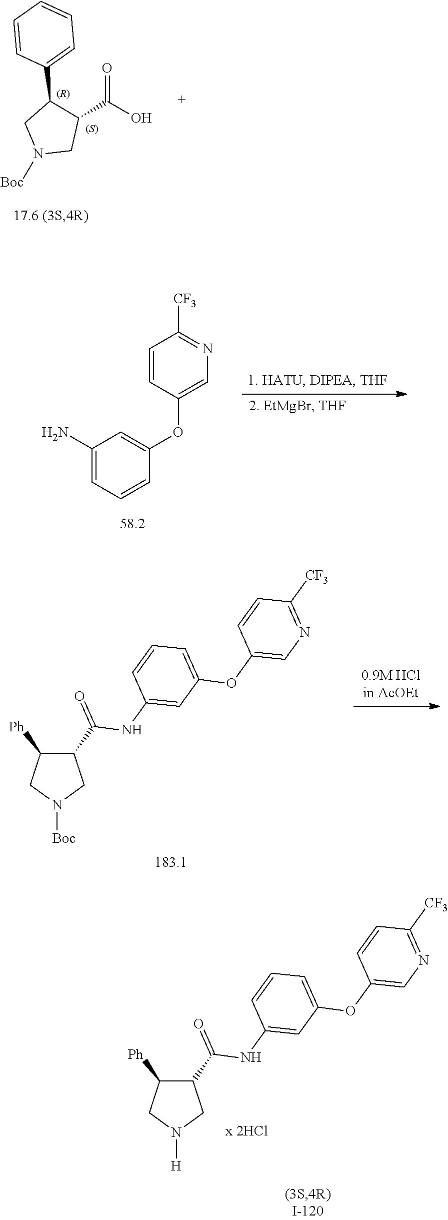

### Step 1: tert-Butyl-(3S,4R)-3-{[(6-trifluoromethylpyridin-3-yl)oxy]phenyl}carbamoyl]-4-phenylpyrrolidine-1-carboxylate (183.1)

Intermediate 183.1 was prepared according to the procedure described in Step 1 of Example 64 starting from intermediate 17.6 (210 mg, 0.72 mmol), HATU (356.3 mg, 0.93 mmol), DIPEA (0.38 mL, 2.16 mmol), 3.0 M EtMgBr in Et₂O (0.24 mL, 0.72 mmol), and intermediate 58.2 (200 mg, 0.79 mmol) in THF (5.0 mL+5.0 mL). Stirring was continued at r.t. for 16 h. After purification by flash chromatography (PET/EtOAc from 100% PET to 65:35 v/v PET/EtOAc) the intermediate 183.1 (120 mg, 0.22 mmol) was obtained. Yield: 32%. MS-ESI(−) m/z: 526.1 (M−H).

### Step 2: (3S,4R)-4-Phenyl-N-(3-{[6-(trifluoromethyl)pyridin-3-yl]oxy}phenyl) pyrrolidine-3-carboxamide dihydrochloride (Compound I-120)

Compound I-120 was prepared following the procedure described in Step 2 of Example 120 starting from a solution of intermediate 183.1 (100 mg, 0.19 mmol) and a 0.9 M solution of HCl in EtOAc (0.94 mL). The title Compound I-120 (60 mg, 0.11 mmol) was obtained as a pale grey solid. Yield: 63%. (400 MHz, DMSO-d₆) δ 3.25-3.30 (m, 2H), 3.38-3.44 (m, 1H), 3.66-3.72 (m, 3H), 6.86-6.88 (m, 1H), 7.24-7.28 (m, 1H), 7.31-7.40 (m, 6H), 7.55 (d, J=8 Hz, 1H), 7.89 (d, J=12 Hz, 1H), 8.54 (d, J=4 Hz, 1H), 9.45 (brs, 1H), 9.86 (brs, 1H), 10.57 (s, 1H). HPLC purity: ≥95%. MS-ESI(+) m/z: 428.1 (M+H).

### Example 184: (3S,4R)-3-{[1-(4-Fluorophenyl)Ipiperidin-4-yl]carbamoyl]}-4-phenylpyrrolidine dihydrochloride (Compound I-121)

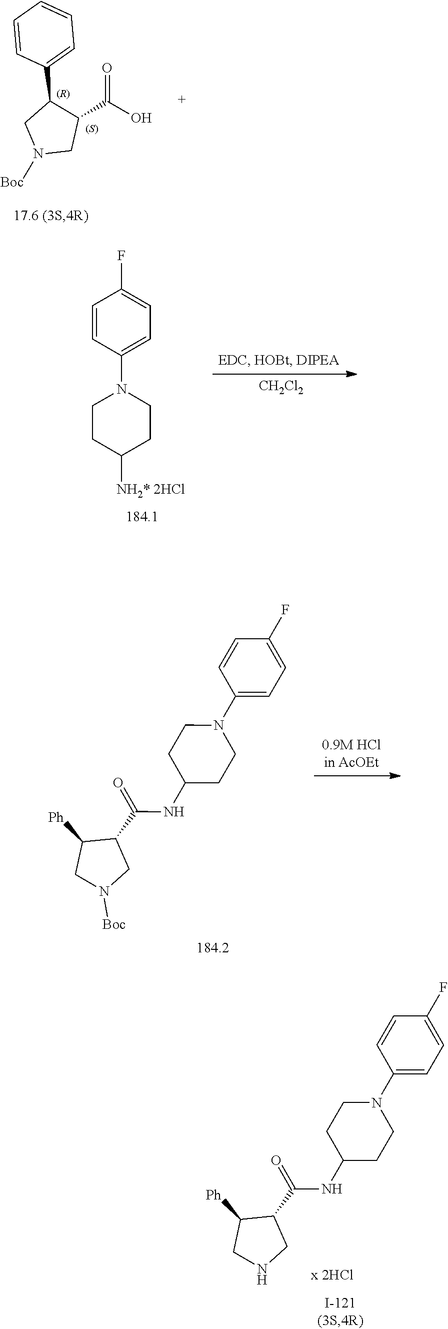

### Step 1: tert-Butyl-(3S,4R)-3-{[1-(4-fluorophenyl)lpiperidin-4-yl]carbamoyl]}-4-phenylpyrrolidine-1 carboxylate (184.2)

Intermediate 184.2 was prepared according to the procedure described in Example 94 starting from a solution of intermediate 17.6 (66 mg, 0.22 mmol), EDC (47 mg, 0.25 mmol), DIPEA (0.17 mL, 1.00 mmol), and intermediate 184.1 (60 mg, 0.22 mmol) in DCM (5 mL). The intermediate 184.2 (22 mg, 0.047 mmol) was obtained after work-up and chromatographic purification (PET/EtOAc, from 85:15 to 30:70 v/v) as a colorless oil. Yield: 21%. ESI(+) m/z: 412.2 (M+H-56), ESI(−) m/z: 466.6 (M−H).

### Step 2: (3S,4R)-3-{[1-(4-Fluorophenyl)lpiperidin-4-yl]carbamoyl]}-4-phenylpyrrolidine dihydrochloride (Compound I-727)

Compound I-121 was prepared following the procedure described in Step 2 of Example 120 starting from a solution of intermediate 184.2 (22 mg, 0.047 mmol) and a 1.0 M solution of HCl in EtOAc (0.47 mL). The title Compound I-121 (19 mg, 0.043 mmol) was obtained as a white solid. Yield: 92%. ¹H NMR (400 MHz, DMSO-d₆) δ 1.59-2.15 (m, 3H), 3.25-3.18 (m, 12H), 7.25-7.34 (m, 7H), 7.45-7.83 (m, 2H), 8.48 (brs, 1H), 9.53 (s, 1H), 9.78 (s, 1H). HPLC purity: ≥95%. MS-ESI(+) m/z: 368.1 (M+H).

### Example 185: (3S,4R)-3-{[1-(4-Cyanophenyl)lpiperidin-4-yl]carbamoyl]}-4-phenylpyrrolidine dihydrochloride (Compound I-122)

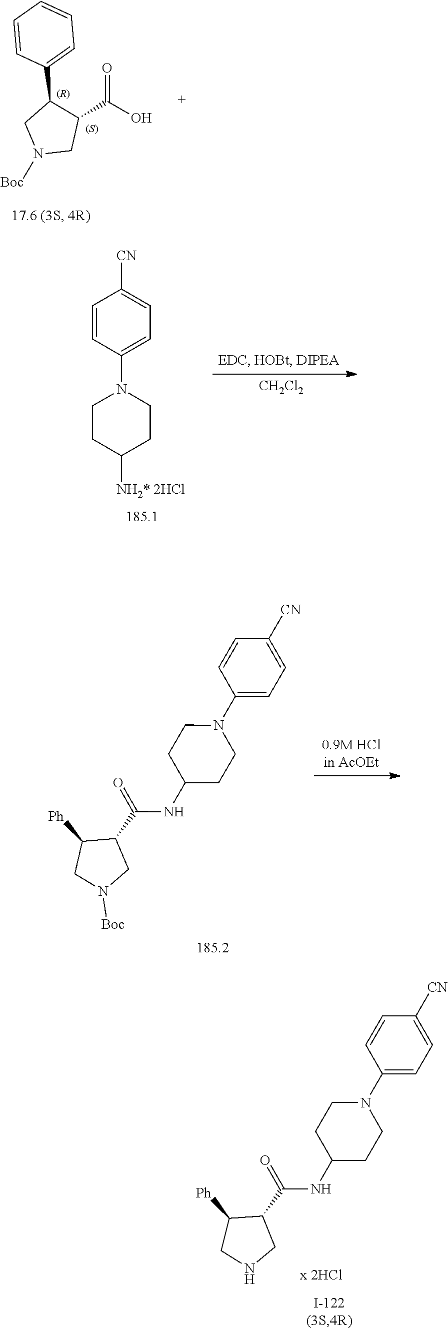

### Step 1: tert-Butyl-(3S,4R)-3-{[1-(4-cyanophenyl)lpiperidin-4-yl]carbamoyl]}-4-phenylpyrrolidine-1 carboxylate (185.2)

Intermediate 185.2 was prepared according to the procedure described in Example 94 starting from a solution of intermediate 17.6 (250 mg, 0.86 mmol), EDC (181 mg, 0.94 mmol), DIPEA (0.59 mL, 3.44 mmol), and intermediate 185.1 (236 mg, 0.86 mmol) in DCM (15 mL). The intermediate 185.2 (180 mg, 0.38 mmol) was obtained after work-up and chromatographic purification (PET/EtOAc, from 100% PET to 45:55 v/v PET/EtOAc) as a white solid. Yield: 44%. ESI(+) m/z: 516.1 (M+H).

### Step 2: 3S,4R)-3-{[1-(4-Cyanophenyl)lpiperidin-4-yl]carbamoyl]}-4-phenylpyrrolidine dihydrochloride Compound I-122)

Compound I-122 was prepared following the procedure described in Step 2 of Example 120 starting from a solution of intermediate 185.2 (160 mg, 0.34 mmol) and a 0.9 M solution of HCl in EtOAc (1.7 mL). The title Compound I-122 (136 mg, 0.3 mmol) was obtained as a white solid. Yield: 90%.

¹H NMR (400 MHz, DMSO-d₆) δ 1.15-1.22 (m, 1H), 1.36-1.39 (m, 1H), 1.58 (m, 1H), 1.73 (m, 1H), 2.92-3.02 (m, 2H), 3.09-3.13 (m, 1H), 3.22-3.24 (m, 2H), 3.57-3.67 (m, 5H), 3.74-3.77 (m, 2H), 6.97 (d, J=8 Hz, 2H), 7.26-7.33 (m, 5H), 7.52 (d, J=8 Hz, 2H), 8.07 (m, 1H), 9.53 (brs, 1H), 9.90 (brs, 1H). HPLC purity: ≥95%. MS-ESI(+) m/z: 416.1 (M+H).

### Example 186: (3S,4R)-3-[(l-Phenylpiperidin-4-yl)carbamoyl]-4-phenylpyrrolidine dihydrochloride (Compound I-123)

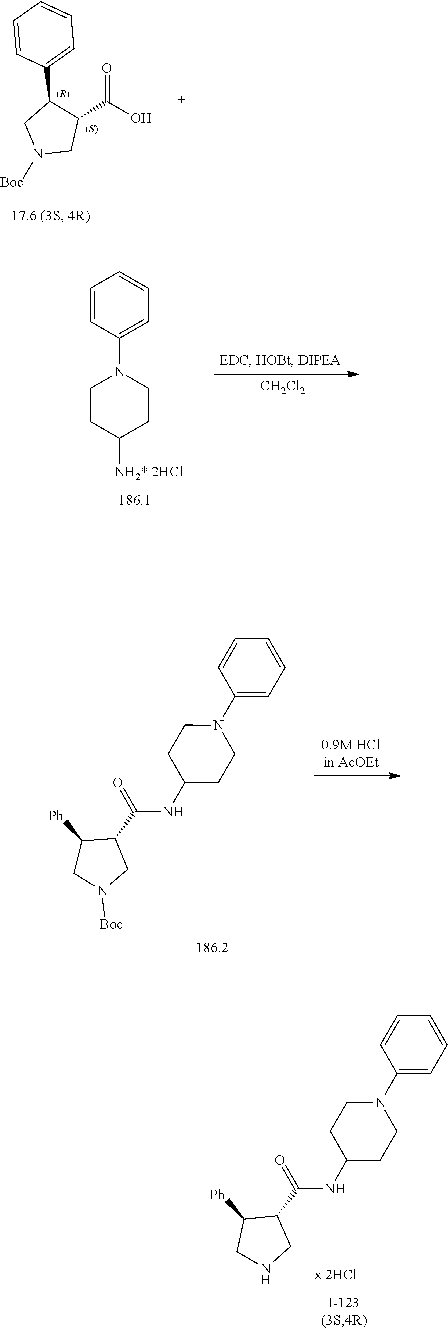

### Step 1: tert-Butyl-(3S,4R)-3-[(1-phenylpiperidin-4-yl)carbamoyl]-4-phenylpyrrolidine-1 carboxylate (186.2)

Intermediate 186.2 was prepared according to the procedure described in Example 94 starting from a solution of intermediate 17.6 (125 mg, 0.43 mmol), EDC (91 mg, 0.47 mmol), DIPEA (0.34 mL, 1.93 mmol), and intermediate 186.1 (106 mg, 0.43 mmol) in DCM (10 mL). The intermediate 186.2 (51 mg, 0.11 mmol) was obtained after work-up and chromatographic purification (PET/EtOAc, from 85:15 to 30:70 v/v) as a colorless oil. Yield: 26%. ESI(+) m/z: 394.2 (M+H-56), ESI(−) m/z: 448.2 (M−H).

### Step 1: (3S,4R)-3-[(I-phenylpiperidin-4-yl)carbamoyl]-4-phenylpyrrolidine dihydrochloride (Compound I-123)

Compound I-123 was prepared following the procedure described in Step 2 of Example 120 starting from a solution of intermediate 186.2 (51 mg, 0.11 mmol) and a 1.0 M solution of HCl in EtOAc (1.13 mL). The title Compound I-123 (48 mg, 0.11 mmol) was obtained as a white solid. Yield: 95%. ¹H NMR (400 MHz, DMSO-d₆) δ 1.70-2.01 (m, 3H), 3.01-3.25 (m, 3H), 3.27-3.51 (m, 1H), 3.53-3.98 (m, 8H), 7.25-7.50 (m, 8H), 7.50-7.80 (m, 2H), 8.46 (brs, 1H), 9.48 (s, 1H), 9.75 (s, 1H).

HPLC purity: ≥95%. MS-ESI(+) m/z: 350.1 (M+H).

### Biological Activity

### In Vitro Assays

### Biochemical NR2F6 Binding Assay

Purified recombinant human NR2F6 protein was dissolved in immobilization buffer at pH 4.5 and then immobilized in EnSpire-LFB High-Sensitivity User-Activated Plates at the concentration of 50 μg/well plate and left in incubation for 16 h at 4° C. Wells with only immobilization buffer were used as negative control to check the protein coating efficiency. NR2F6 ligands were resuspended in DMSO at the concentration of 10 mM, then dispensed into a plate containing assay buffer at concentrations ranging from 1 nM to 100 uM using an HP-dispenser. DMSO was normalized at the 1% of volume of each well. The final readings were taken over a period of 30 minutes. The label-free responses were measured as shifts in reflected wavelength and are expressed in picometers (pm). Results were analyzed using the EnSpire label-free user interface software, and EC₅₀ and graphs were generated using GraphPad PRISM software. The results are shown in tables below. In the tables below, A is <1 μM; B is 1 to 10 μM; C is 10 to 50 μM; and D is >50 μM.

| Compound | | |
|---|---|---|
| No. | Structure | EC₅₀ |
| I-1 | 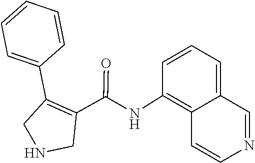 [SMILES: O=C(Nc1cccc2cnccc12)C1=C(c2ccccc2)CNC1] | C |
| I-2 | 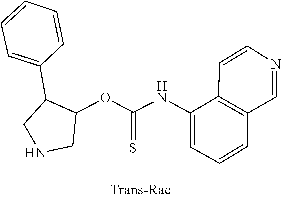 [SMILES: S=C(Nc1cccc2cnccc12)OC1CNCC1c1ccccc1] | B |
| I-3 | 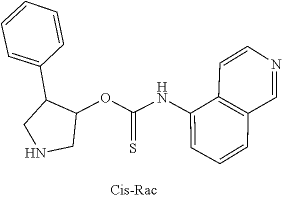 [SMILES: S=C(Nc1cccc2cnccc12)OC1CNCC1c1ccccc1] | C |
| I-4 | 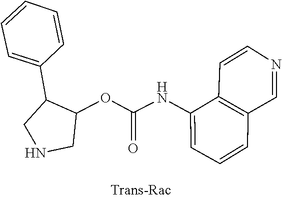 [SMILES: O=C(Nc1cccc2cnccc12)OC1CNCC1c1ccccc1] | B |
| I-5 | 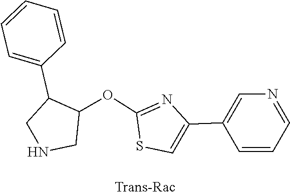 [SMILES: c1ccc(C2CNCC2Oc2nc(-c3cccnc3)cs2)cc1] | B |
| I-6 | 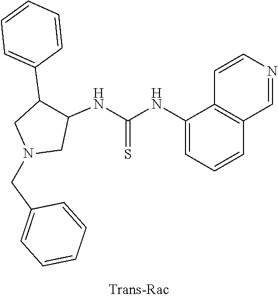 [SMILES: S=C(Nc1cccc2cnccc12)NC1CN(Cc2ccccc2)CC1c1ccccc1] | B |
| I-7 | 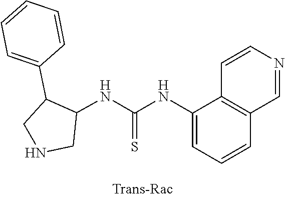 [SMILES: S=C(Nc1cccc2cnccc12)NC1CNCC1c1ccccc1] | B |
| I-8 | 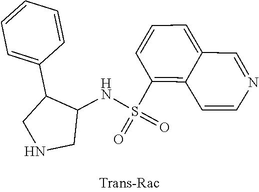 [SMILES: O=S(=O)(NC1CNCC1c1ccccc1)c1cccc2cnccc12] | D |
| I-9 | 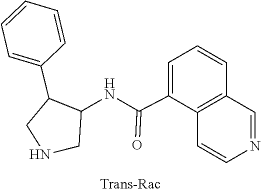 [SMILES: O=C(NC1CNCC1c1ccccc1)c1cccc2cnccc12] | B |
| I-10 | 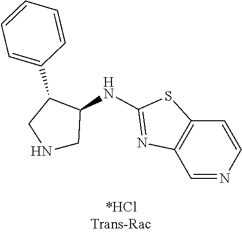 [SMILES: c1ccc([C@H]2CNC[C@@H]2Nc2nc3cnccc3s2)cc1] | D |
| I-11 | 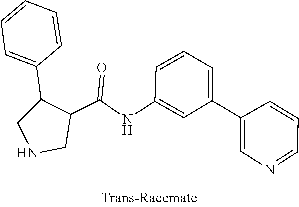 [SMILES: O=C(Nc1cccc(-c2cccnc2)c1)C1CNCC1c1ccccc1] | B |
| I-12 | 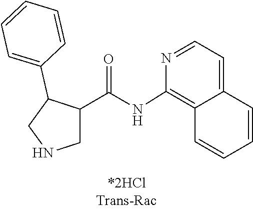 [SMILES: O=C(Nc1nccc2ccccc12)C1CNCC1c1ccccc1] | D |
| I-13 | 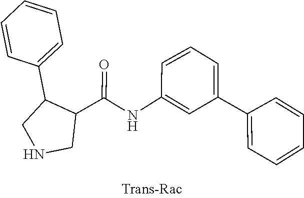 [SMILES: O=C(Nc1cccc(-c2ccccc2)c1)C1CNCC1c1ccccc1] | B |
| I-14 | 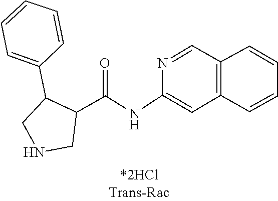 [SMILES: O=C(Nc1cc2ccccc2cn1)C1CNCC1c1ccccc1] | B |
| I-15 | 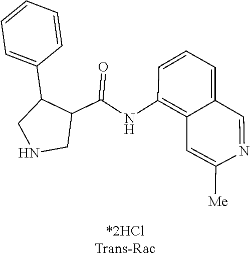 [SMILES: Cc1cc2c(NC(=O)C3CNCC3c3ccccc3)cccc2cn1] | B |
| I-16 | 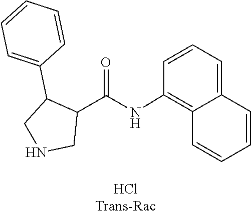 [SMILES: O=C(Nc1cccc2ccccc12)C1CNCC1c1ccccc1] | B |
| I-17 | 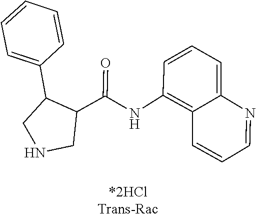 [SMILES: O=C(Nc1cccc2ncccc12)C1CNCC1c1ccccc1] | C |
| I-18 | 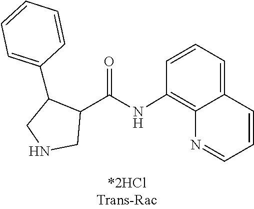 [SMILES: O=C(Nc1cccc2cccnc12)C1CNCC1c1ccccc1] | C |
| I-19 | 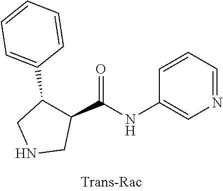 [SMILES: O=C(Nc1cccnc1)[C@H]1CNC[C@@H]1c1ccccc1] | D |
| I-20 | 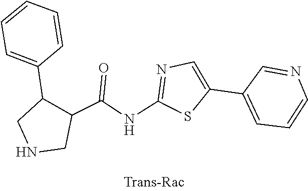 [SMILES: O=C(Nc1ncc(-c2cccnc2)s1)C1CNCC1c1ccccc1] | B |
| I-21 | 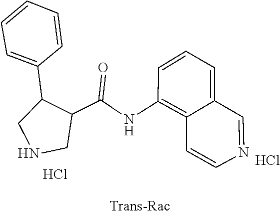 [SMILES: O=C(Nc1cccc2cnccc12)C1CNCC1c1ccccc1 ; Cl ; Cl] | B |
| I-22 | 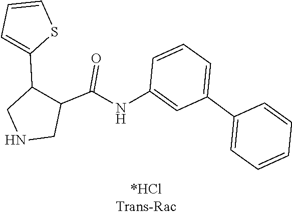 [SMILES: O=C(Nc1cccc(-c2ccccc2)c1)C1CNCC1c1cccs1] | A |
| I-23 | 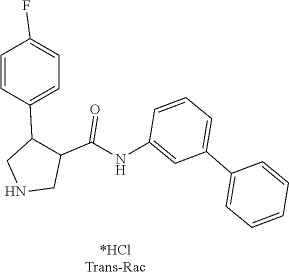 [SMILES: O=C(Nc1cccc(-c2ccccc2)c1)C1CNCC1c1ccc(F)cc1] | B |
| I-24 | 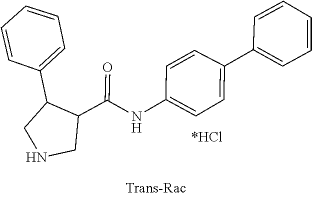 [SMILES: O=C(Nc1ccc(-c2ccccc2)cc1)C1CNCC1c1ccccc1] | B |
| I-25 | 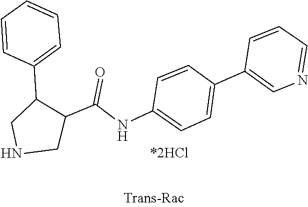 [SMILES: O=C(Nc1ccc(-c2cccnc2)cc1)C1CNCC1c1ccccc1] | B |
| I-26 | 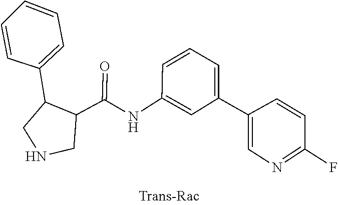 [SMILES: O=C(Nc1cccc(-c2ccc(F)nc2)c1)C1CNCC1c1ccccc1] | B |
| I-27 | 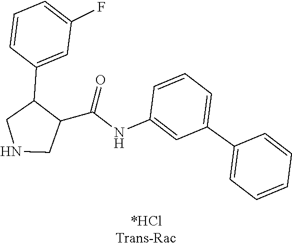 [SMILES: O=C(Nc1cccc(-c2ccccc2)c1)C1CNCC1c1cccc(F)c1] | D |
| I-28 | 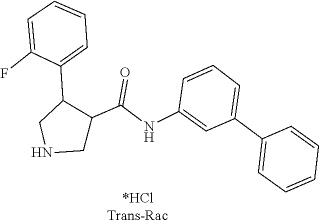 [SMILES: O=C(Nc1cccc(-c2ccccc2)c1)C1CNCC1c1ccccc1F] | D |
| I-29 | 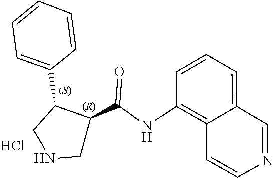 | A |
| I-30 | 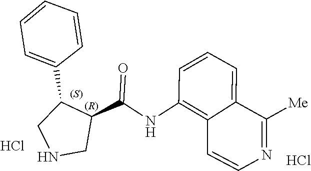 | A |
| I-31 | 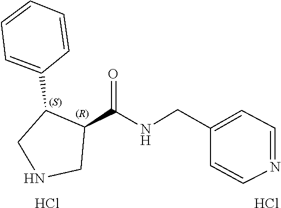 | B |
| I-32 | 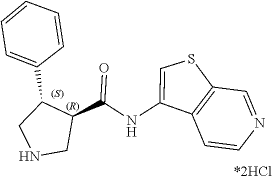 | B |
| I-33 | 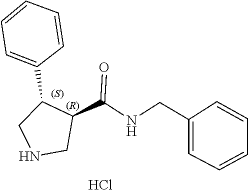 | C |
| I-34 | 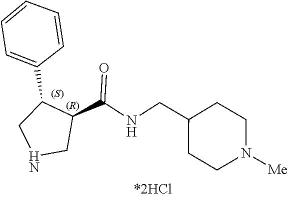 | C |
| I-35 | 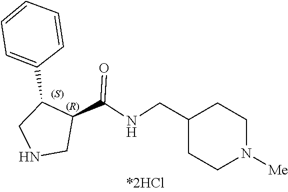 | D |
| I-36 | 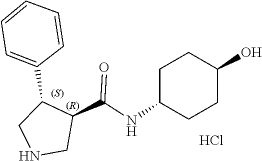 | D |
| I-37 | 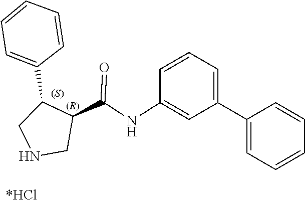 | B |
| I-38 | 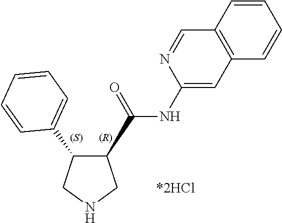 | B |
| I-39 | 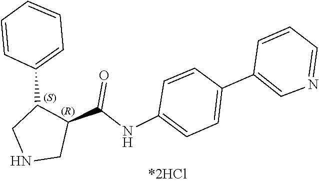 | D |
| I-40 | 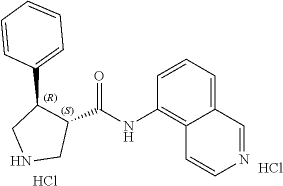 | D |
| I-41 | 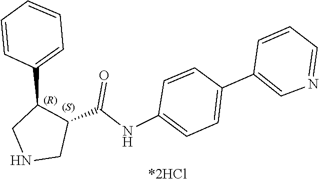 | B |
| I-42 | 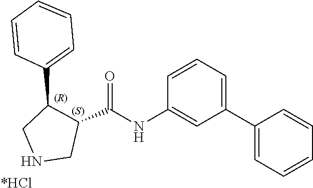 | B |
| I-43 | 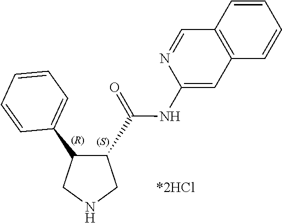 | B |
| I-44 | 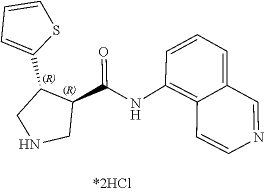 | D |
| I-45 | 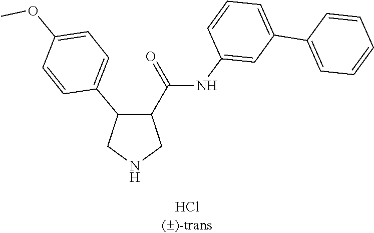 [SMILES: COc1ccc(C2CNCC2C(=O)Nc2cccc(-c3ccccc3)c2)cc1] | D |
| I-46 | 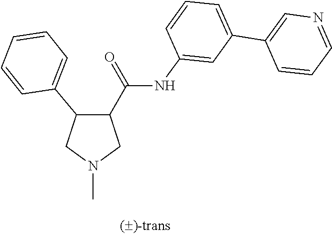 [SMILES: CN1CC(C(=O)Nc2cccc(-c3cccnc3)c2)C(c2ccccc2)C1] | D |
| I-47 | 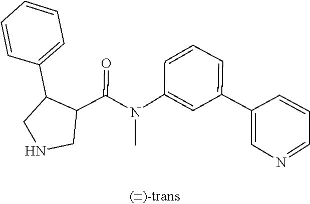 [SMILES: CN(C(=O)C1CNCC1c1ccccc1)c1cccc(-c2cccnc2)c1] | D |
| I-48 | 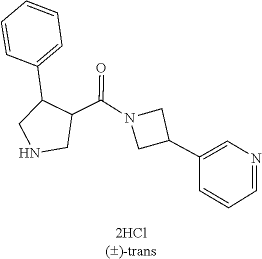 [SMILES: O=C(C1CNCC1c1ccccc1)N1CC(c2cccnc2)C1] | D |
| I-49 | 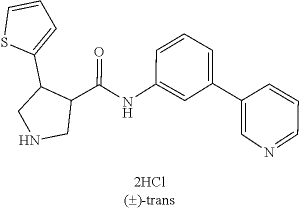 [SMILES: O=C(Nc1cccc(-c2cccnc2)c1)C1CNCC1c1cccs1] | D |
| I-50 | 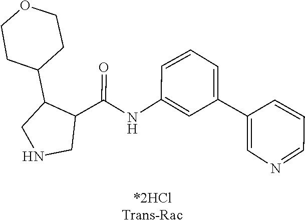 [SMILES: O=C(Nc1cccc(-c2cccnc2)c1)C1CNCC1C1CCOCC1] | D |
| I-51 | 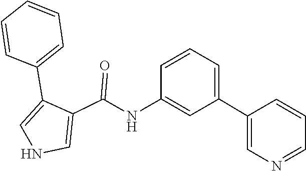 [SMILES: O=C(Nc1cccc(-c2cccnc2)c1)c1c[nH]cc1-c1ccccc1] | D |
| I-52 | 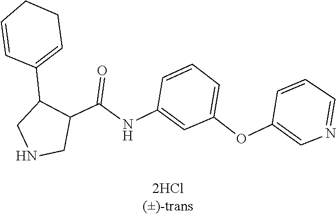 [SMILES: O=C(Nc1cccc(Oc2cccnc2)c1)C1CNCC1C1=CCCC=C1] | D |
| I-53 | 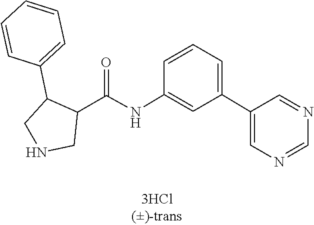 [SMILES: O=C(Nc1cccc(-c2cncnc2)c1)C1CNCC1c1ccccc1] | D |
| I-54 | 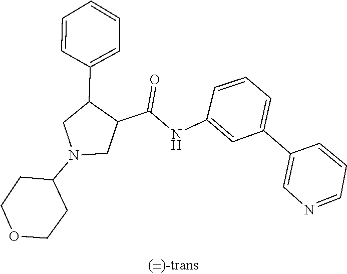 [SMILES: O=C(Nc1cccc(-c2cccnc2)c1)C1CN(C2CCOCC2)CC1c1ccccc1] | D |
| I-55 | 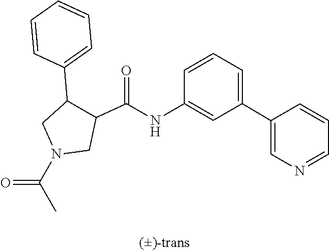 [SMILES: CC(=O)N1CC(C(=O)Nc2cccc(-c3cccnc3)c2)C(c2ccccc2)C1] | D |
| I-56 | 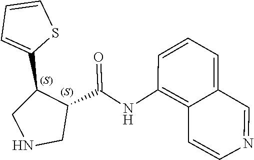 | D |
| I-57 | 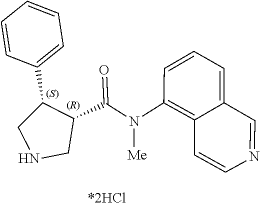 | D |
| I-58 | 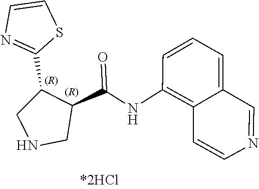 | D |
| I-59 | 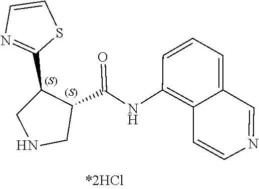 | D |

Additional compounds and data are shown below.

| Compound | | |
|---|---|---|
| No. | Structure | EC₅₀ |
| I-60 | 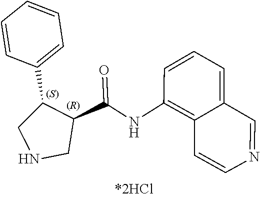 | B |
| I-61 | 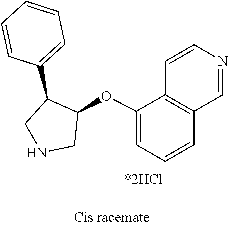 [SMILES: c1ccc([C@@H]2CNC[C@@H]2Oc2cccc3cnccc23)cc1] | B |
| I-62 | 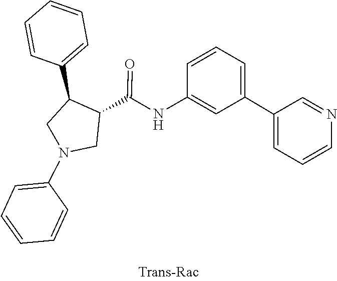 [SMILES: O=C(Nc1cccc(-c2cccnc2)c1)[C@@H]1CN(c2ccccc2)C[C@H]1c1ccccc1] | A |
| I-63 | 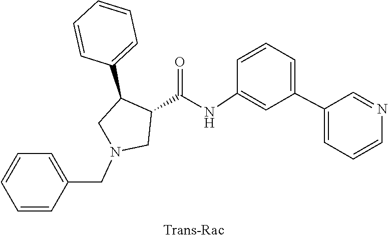 [SMILES: O=C(Nc1cccc(-c2cccnc2)c1)[C@@H]1CN(Cc2ccccc2)C[C@H]1c1ccccc1] | B |
| I-64 | 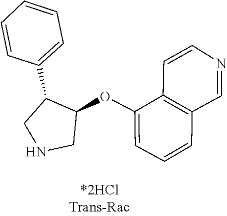 [SMILES: c1ccc([C@H]2CNC[C@@H]2Oc2cccc3cnccc23)cc1] | B |
| I-65 | 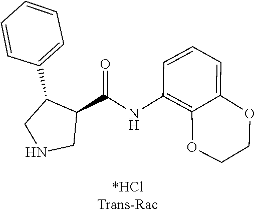 [SMILES: O=C(Nc1cccc2c1OCCO2)[C@H]1CNC[C@@H]1c1ccccc1] | D |
| I-66 | 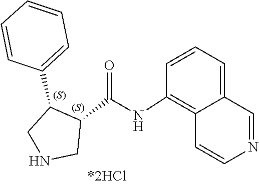 | D |
| I-67 | 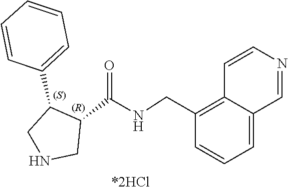 | D |
| I-68 | 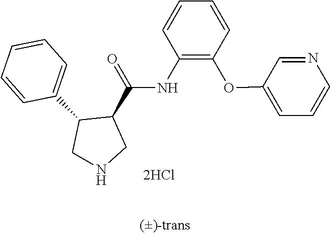 [SMILES: O=C(Nc1ccccc1Oc1cccnc1)[C@H]1CNC[C@@H]1c1ccccc1] | B |
| I-69 | 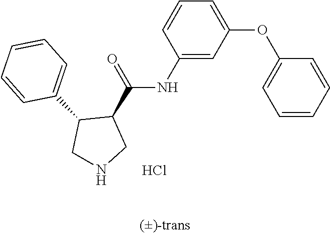 [SMILES: O=C(Nc1cccc(Oc2ccccc2)c1)[C@H]1CNC[C@@H]1c1ccccc1 ; Cl] | A |
| I-70 | 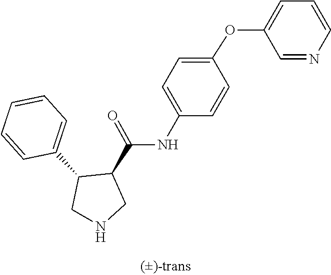 [SMILES: O=C(Nc1ccc(Oc2cccnc2)cc1)[C@H]1CNC[C@@H]1c1ccccc1] | D |
| I-71 | 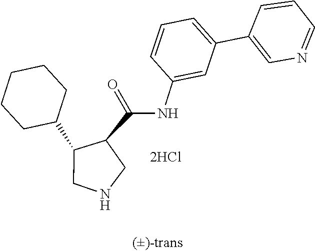 [SMILES: O=C(Nc1cccc(-c2cccnc2)c1)[C@H]1CNC[C@@H]1C1CCCCC1] | B |
| I-72 | 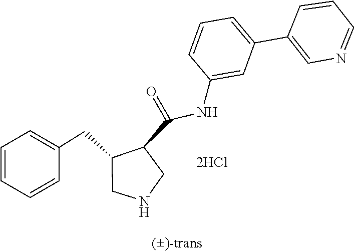 [SMILES: O=C(Nc1cccc(-c2cccnc2)c1)[C@H]1CNC[C@@H]1Cc1ccccc1] | D |
| I-73 | 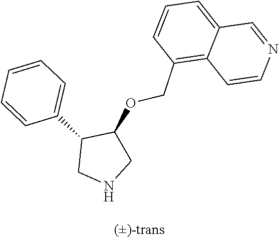 [SMILES: c1ccc([C@H]2CNC[C@@H]2OCc2cccc3cnccc23)cc1] | D |
| I-74 | 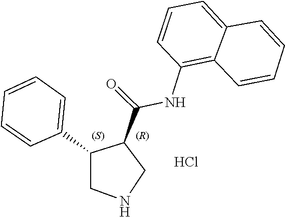 | B |
| I-75 | 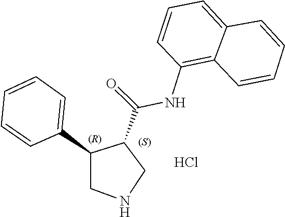 | B |
| I-76 | 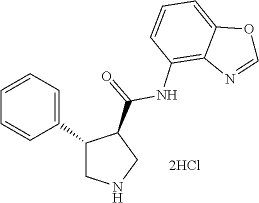 [SMILES: O=C(Nc1cccc2ocnc12)[C@H]1CNC[C@@H]1c1ccccc1] | D |
| I-77 | 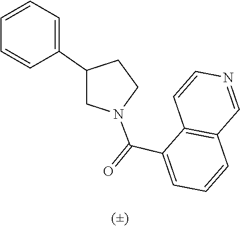 [SMILES: O=C(c1cccc2cnccc12)N1CCC(c2ccccc2)C1] | D |
| I-78 | 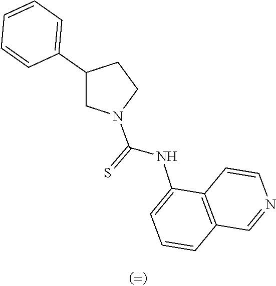 [SMILES: S=C(Nc1cccc2cnccc12)N1CCC(c2ccccc2)C1] | D |
| I-79 | 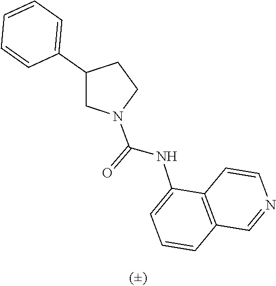 [SMILES: O=C(Nc1cccc2cnccc12)N1CCC(c2ccccc2)C1] | D |
| I-80 | 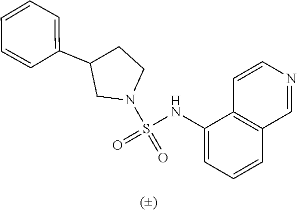 [SMILES: O=S(=O)(Nc1cccc2cnccc12)N1CCC(c2ccccc2)C1] | D |
| I-81 | 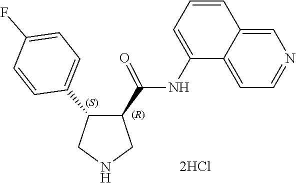 | D |
| I-82 | 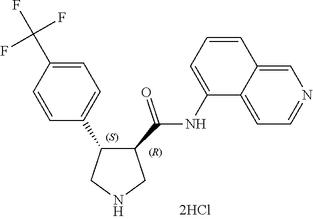 | D |
| I-83 | 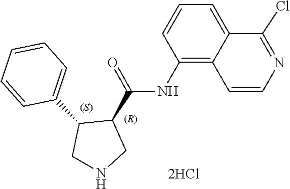 | B |
| I-84 | 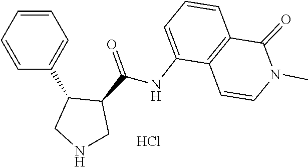 [SMILES: Cn1ccc2c(NC(=O)[C@H]3CNC[C@@H]3c3ccccc3)cccc2c1=O ; Cl] | D |
| I-85 | 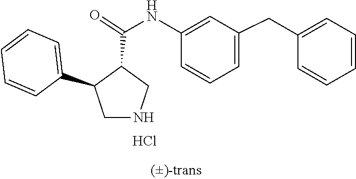 [SMILES: O=C(Nc1cccc(Cc2ccccc2)c1)[C@@H]1CNC[C@H]1c1ccccc1 ; Cl] | B |
| I-86 | 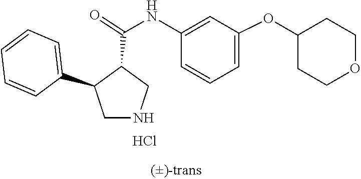 [SMILES: O=C(Nc1cccc(OC2CCOCC2)c1)[C@@H]1CNC[C@H]1c1ccccc1 ; Cl] | B |
| I-87 | 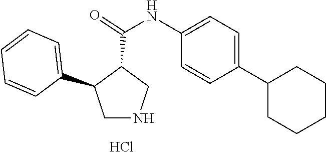 [SMILES: O=C(Nc1ccc(C2CCCCC2)cc1)[C@@H]1CNC[C@H]1c1ccccc1 ; Cl] | B |
| I-88 | 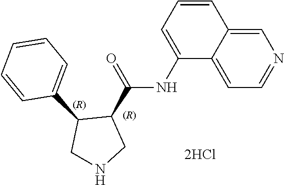 | B |
| I-89 | 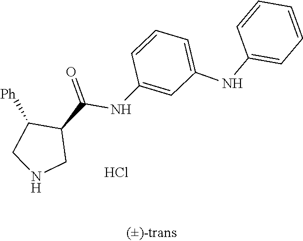 [SMILES: O=C(Nc1cccc(Nc2ccccc2)c1)[C@H]1CNC[C@@H]1c1ccccc1 ; Cl] | A |
| I-90 | 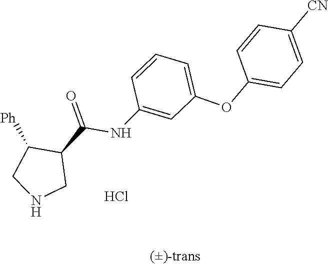 [SMILES: N#Cc1ccc(Oc2cccc(NC(=O)[C@H]3CNC[C@@H]3c3ccccc3)c2)cc1 ; Cl] | A |
| I-91 | 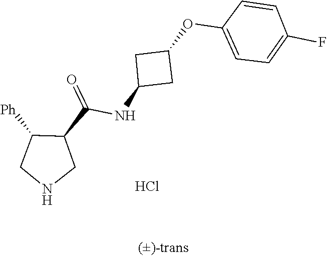 [SMILES: O=C(N[C@H]1C[C@H](Oc2ccc(F)cc2)C1)[C@H]1CNC[C@@H]1c1ccccc1 ; Cl] | B |
| I-92 | 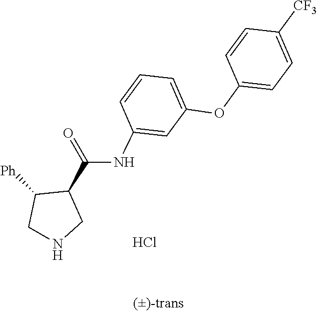 [SMILES: O=C(Nc1cccc(Oc2ccc(C(F)(F)F)cc2)c1)[C@H]1CNC[C@@H]1c1ccccc1 ; Cl] | B |
| I-93 | 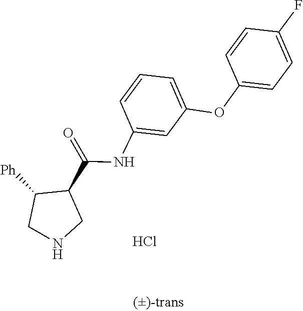 [SMILES: O=C(Nc1cccc(Oc2ccc(F)cc2)c1)[C@H]1CNC[C@@H]1c1ccccc1 ; Cl] | A |
| I-94 | 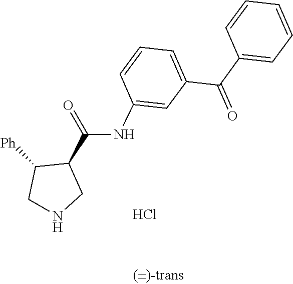 [SMILES: O=C(c1ccccc1)c1cccc(NC(=O)[C@H]2CNC[C@@H]2c2ccccc2)c1 ; Cl] | B |
| I-95 | 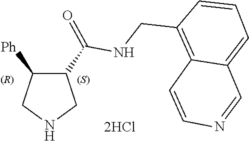 | B |
| I-96 | 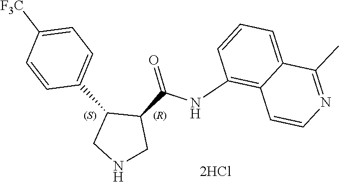 | B |
| I-97 | 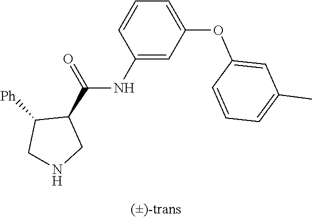 [SMILES: Cc1cccc(Oc2cccc(NC(=O)[C@H]3CNC[C@@H]3c3ccccc3)c2)c1] | B |
| I-98 | 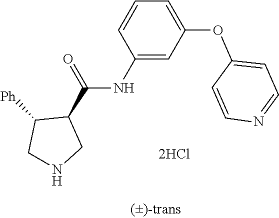 [SMILES: O=C(Nc1cccc(Oc2ccncc2)c1)[C@H]1CNC[C@@H]1c1ccccc1] | D |
| I-99 | 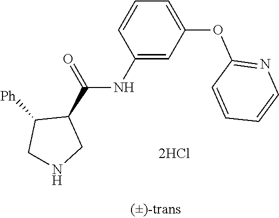 [SMILES: O=C(Nc1cccc(Oc2ccccn2)c1)[C@H]1CNC[C@@H]1c1ccccc1] | D |
| I-100 | 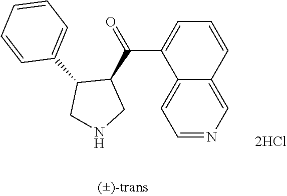 [SMILES: O=C(c1cccc2cnccc12)[C@H]1CNC[C@@H]1c1ccccc1] | B |
| I-101 | 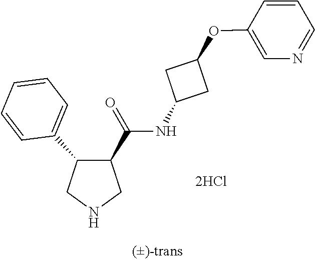 [SMILES: O=C(N[C@H]1C[C@H](Oc2cccnc2)C1)[C@H]1CNC[C@@H]1c1ccccc1] | C |
| I-102 | 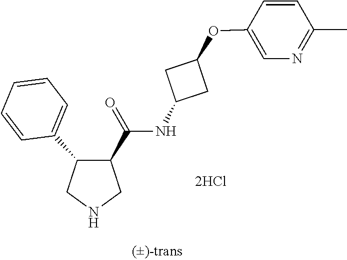 [SMILES: Cc1ccc(O[C@H]2C[C@H](NC(=O)[C@H]3CNC[C@@H]3c3ccccc3)C2)cn1] | B |
| I-103 | 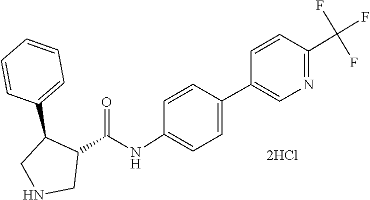 | A |
| I-104 | 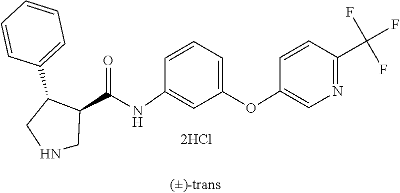 [SMILES: O=C(Nc1cccc(Oc2ccc(C(F)(F)F)nc2)c1)[C@H]1CNC[C@@H]1c1ccccc1] | A |
| I-105 | 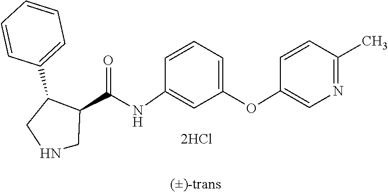 [SMILES: Cc1ccc(Oc2cccc(NC(=O)[C@H]3CNC[C@@H]3c3ccccc3)c2)cn1] | B |
| I-106 | 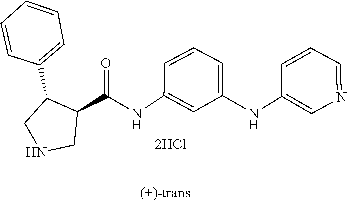 [SMILES: O=C(Nc1cccc(Nc2cccnc2)c1)[C@H]1CNC[C@@H]1c1ccccc1] | A |
| I-107 | 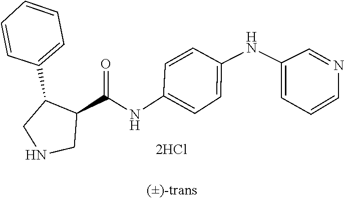 [SMILES: O=C(Nc1ccc(Nc2cccnc2)cc1)[C@H]1CNC[C@@H]1c1ccccc1] | D |
| I-108 | 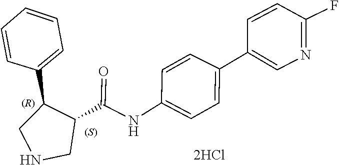 | A |
| I-109 | 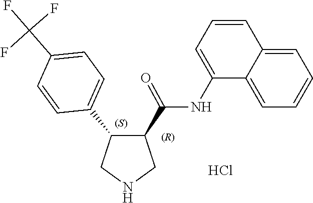 | B |
| I-110 | 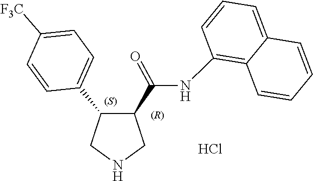 | B |
| I-111 | 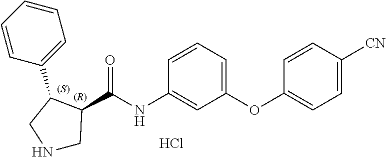 | A |
| I-112 | 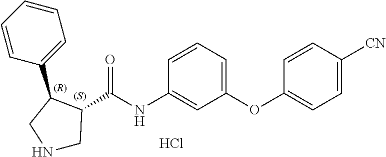 | A |
| I-113 | 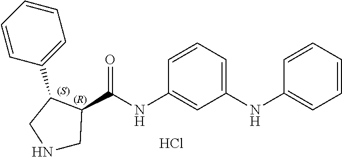 | B |
| I-114 | 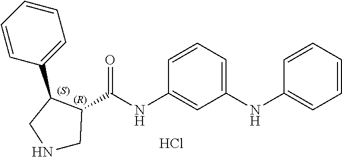 | B |
| I-115 | 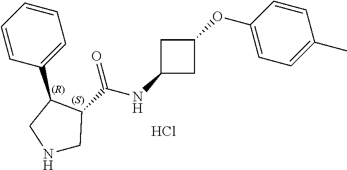 | B |
| I-116 | 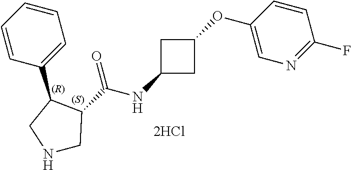 | C |
| I-117 | 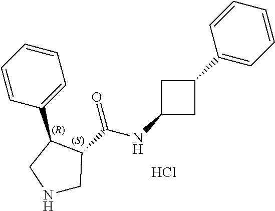 | B |
| I-118 | 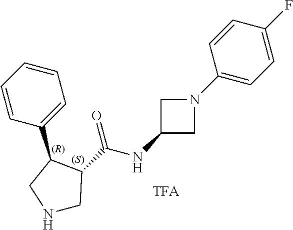 | A |
| I-119 | 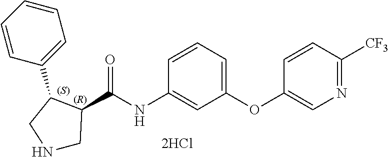 | B |
| I-120 | 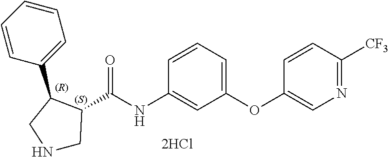 | B |
| I-121 | 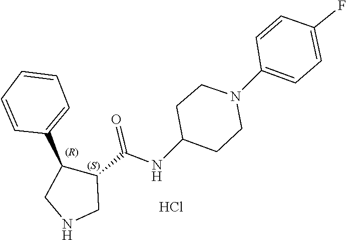 | B |
| I-122 | 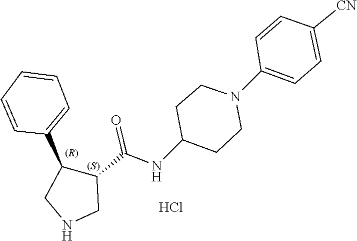 | A |
| I-123 | 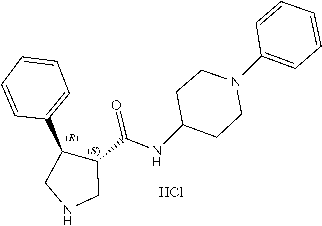 | B |

### Inflammation Assay

RAW 264.7 cells (murine macrophage cell line) are purchased from the American Type Tissue Culture Collection (ATCC, Rockville, Md.). RAW 264.7 cells are suspended in complete medium; DMEM supplemented with 10% fetal bovine serum (FBS), 100 U/ml penicillin, and 100 U/ml streptomycin. Cells are plated into 24-well plates at a density of 5×105 cells/well. All experiments are performed in a humidified atmosphere under 5% CO₂ at 37° C. LPS is purchased from Sigma (St. Louis, Mo.). TNF-alpha expression in RAW 264.7 macrophages is measured following NR2F6 ligands treatment alone or in combination with LPS-stimulation for 2 hr. LPS is used at a final concentration of 6 ng/mL.

### RT-PCR

Total RNA from Raw 264.7 cell lines is extracted using the RNAesy Plus (Quiagen) and then reverse-transcribed using Vilo enzyme (Life-Technologies. The RT-PCR reactions are performed using SYBR Green on a CFX96 real time system (BioRad). The comparative Ct (ΔΔCt) method is used to determine the fold-change in expression using B2M (beta-2 microglobulin) as the reference gene for normalization.

### Cytokine Detection

CD4⁺ T cells are purified from the spleens of C57BL/6 male mice. Spleens are processed and erythrocytes removed by specific lysis buffer. Total splenocytes are incubated with an antibody cocktail containing biotin bound B220 and cD11c antibodies. After washing cells are incubated again with streptavidin beads and passed into magnetic column in order to elute B220-CD11c cell fraction. B220/CD11c negative cells are used to isolate CD4⁺ T cells by using CD4-magnetic beads. After this incubation, cells are sorted using a new magnetic column and the positive CD4 fraction is eluted using a specific elution buffer and detaching the column from the magnet. CD4⁺ T cell purity is assessed by FACS analysis and routinely ranging between 90-95%. Then the cells are re-suspended at concentration of 0.5×10⁶/ml and stimulated with plate bound anti-CD3 and anti-CD28 under T helper differentiation conditions.

CD4+Th0 cells are stimulated with NR2F6 ligands for 24 hrs and supernatant is harvested and stored at −80° C. Cells are also stimulated with DMSO alone. Cytokine secretions are monitored using BioPlex Luminex Technology according to manufacturer's instructions. BioPlex-200 Instrument is used as the plate reader.

Cytotoxicity and hERG Screening

Cytotoxicity: 20000 HePG2 and AML-12 cells are seeded in 96 well plate (Viewplate PerkinElmer). Dose-response of the compound is performed using HP D300 digital dispenser, ranging from 10 nM to 300 μM with constant DMSO 1% in medium. Cells are stimulated for 4 hrs at 37° C.; the supernatant is used to perform LDH release (Cytotox-one, Promega) as a measure of necrosis while the cells are lysed to detect ATP level for determining cell viability (Celltiter-glo, Promega) according to manufacturer's instructions.

The Predictor hERG assay kit (Invitrogen), containing membrane preparations from Chinese hamster ovary cells stably transfected with hERG potassium channel and a high-affinity red fluorescent hERG channel ligand (tracer), is used for the determination of hERG channel affinity binding of the test compounds. Compounds that bind to the hERG channel protein (competitors) are identified by their ability to displace the tracer, resulting in a lower fluorescence polarization. The final concentration of DMSO in each well is maintained at 1%. The assays are performed according to the manufacturer's protocol (Invitrogen).

### In Vivo Assays

### Subcutaneous Cancer Mouse Models (Example for B16 Melanoma Model)

1×10⁵ B16-F10 tumor cells are injected subcutaneously (s.c.) into the left flank of C57BL/6 male mice (8-week-old) obtained from Charles River Breeding Laboratories. Two days after tumor injection, NR2F6 ligands or vehicle are administered intraperitoneal (i.p.) daily for 21 days. Tumor growth is monitored four times a week by measuring tumor length and width. For survival analysis, mice are monitored for up 30 days and then sacrificed.

### Flow Cytometry Analysis

Splenocytes and lymph node cells are mashed through a 40-μm filter. Infiltrating cells are isolated from tumor tissues by mechanical disruption and by digestion with collagenase D and DNase I. Splenocytes, lymph node cells, and TILs are incubated with FcR Block to prevent nonspecific antibody binding before staining with appropriate surface antibodies for 10 min, washed with MACS buffer, and used for FACS analysis. For intracellular cytokine staining, splenocytes and lymph node cells are stimulated with PMA, ionomycin and Brefeldin for 4-5 h. Before staining with specific intracellular antibodies cells are stained with T cell surface markers: CD45-APC-cy7, CD3-APC, CD4-BV510, CD8-Percp cy5.5, CD44-BV786, PD-1-PEcy7. Then, cells are fixed and permeabilized and the following antibodies are used for intracellular staining: IL-2-BV605 and IFN-γ-AF488. Other sets of antibodies are used to detect DCs, macrophages or MDSCs: CD45-Percp, B220-BV786, CD11c APC-cy7, MHCII-BV510, CD172-APC, XCR1-BV650, CD11b-AF700, Ly6G-PEcy7, Ly6C-BV421, GR1-PE. Data acquisition is performed by Fortessa flow cytometer and analyzed by FlowJo analysis software (Tree Star, OR, USA).

### DSS Colitis Model

In vivo model of colitis is induced using 2.5% (w/v) dextran sodium sulfate (DSS, 36,000 to 50,000 MW) dissolved in drinking water given ad libitum for 5 consecutive days. Body weight is evaluated every day. NR2F6 ligands are administered intraperitoneal (i.p.) daily for 12 days. Mice are then sacrificed. Colon length is measured, and histological analysis is carried out. The levels of proinflammatory genes and mRNA expression are determined using real-time PCR.

### Leukemia Mouse Model

NSG adult mice (6-8 weeks old) are sub-lethally irradiated with 250 cGy of total body irradiation or treated with 20 mg/kg busulfan by intraperitoneal administration 24 h before injection of leukemic cells. Cultured human AML cell lines (i.e. MV-4-11) are washed and cleared of aggregates and debris using a 0.2-mm cell filter, and suspended in PBS at a final concentration of 0.2-2 million cells per 200 μl of PBS per mouse for intravenous injection (tail vein). NR2F6 ligands are administered intraperitoneal (i.p.) daily for 28 days. Mice are monitored daily for symptoms of disease (i.e. ruffled coat, hunched back, weakness). Animals with sign of distress are humanely sacrificed.

### Experimental Autoimmune Encephalomyelitis (EAE) Mouse Model

8-11 week old C57BL/6 female mice are immunized by injecting 200 mg of MOG₃₅₋₅₅ peptide in CFA, supplemented with 5 mg/ml Mycobacterium tuberculosis H37 Ra emulsified 1:1 in PBS (200 ml). Moreover, 200 ng of pertussis toxin dissolved in PBS (200 ml) is injected 24 h later intravenously in the mice tail vein. NR2F6 ligands are administered intraperitoneal (i.p.) daily for 21 days. Mice are monitored daily for clinical signs of EAE and scored for disease based on a scale of increasing severity from 0 to 5 (0: health, 5: moribund or death).

### Orthotopic Syngeneic Cancer Mouse Models (Example for ID8-Luc Ovarian Cancer Model)

3×10⁵ ID8-Luc tumor cells were injected into the peritoneal cavity of C57BL6 mice on day 0. After 14 days the mice were treated daily with either vehicle alone or NR2F6 ligands intraperitoneally (i.p.) for 21 days. The tumor growth was detected weekly by monitoring luciferase activity after injection of luciferin. For survival analysis, mice were monitored for up 35 days and then sacrificed.

### Study of the Effects of Compounds on Non-Alcoholic Fatty Liver Disease (NAFLD) and Non-Alcoholic Steatohepatitis (NASH) in Mice

A study is performed to determine the effects of compounds of Formula (I-A), (II-A), (I), (II), or (III), or a pharmaceutically acceptable salt thereof, on non-alcoholic fatty liver disease (NAFLD) and non-alcoholic steatohepatitis (NASH) in male C57BL/6J fed a high fat and high sucrose diet.

Male C57BL/6J mice (The Jackson Laboratory, Bar Harbor, Me., USA) are housed under a 14 hrs light-10 hrs dark cycle at 21-23° C. and have ad libitum access to water during the entire experiment. From the age of 6 weeks, mice are fed a ‘Western’ HF-HSD with 44.6% of kcal derived from fat (of which 61% saturated fatty acids) and 40.6% of kcal derived from carbohydrates (primarily sucrose 340 g/kg diet) (TD.08811, 45% kcal Fat Diet, Harlan Laboratories Inc., Madison, Wis., USA) or normal chow diet (NCD) as control (VI534-000 ssniff R/M-H, ssniff Spezialdiaten GmbH, Soest, Germany). The animals are then treated with a compound of Formula (I-A), (II-A), (I), (II), or (III), or a pharmaceutically acceptable salt thereof, or a control for 4, 12 or 20 weeks (n=8 per group for every time point), after which they are sacrificed.

Body weight and food intake are monitored weekly on the same day. After sedation with sodium pentobarbital (intraperitoneal injection, 50 mg/kg body weight), total fat mass is analyzed by dual-energy X-ray absorptiometry (DEXA) (PIXImus densitometer, Lunar Corp., Madison, Wis., USA). Intraperitoneal glucose tolerance test (IPGTT) is performed in 6 hrs fasted mice. Tail vein glucose levels are measured with a Bayer Contour glucometer immediately before (time point 0 min) and 15, 30, 60, 90 and 150 min after glucose administration (1 g glucose/kg bodyweight). Insulin resistance is calculated using the Homeostasis Model of Insulin Resistance (HOMA-IR) index: (fasting insulin (ng/mL) x fasting glucose (mg/dL))/405.

### Sacrifice

After a 6 hrs fasting period, mice are anaesthetized with sodium pentobarbital (intraperitoneal injection, 50 mg/kg body weight) and sacrificed by blood sampling via cardiac puncture. Plasma is obtained by centrifugation of blood (6000 rpm for 5 min at 4° C.) that is collected in heparinized syringes. Tissues are either snap frozen in liquid nitrogen or stored at −80° C. together with the plasma until further biochemical and molecular analyses or preserved for histological analysis.

### Histological Analyses

Liver samples are routinely fixed in buffered formalin (4%) and embedded in paraffin. Serial 4 mm thick sections are stained with H&E and picrosirius red to assess fibrosis. Frozen liver sections are stained with Oil Red O to assess lipid accumulation. All liver biopsies are analysed by an expert liver pathologist, blinded to the dietary condition or surgical intervention. Steatosis, activity and fibrosis are semi-quantitatively scored according to the NASH-Clinical Research Network criteria. The amount of steatosis (percentage of hepatocytes containing fat droplets) is scored as 0 (<5%), 1 (5-33%), 2 (>33-66%) and 3 (>66%). Hepatocyte ballooning is classified as 0 (none), 1 (few) or 2 (many cells/prominent ballooning). Foci of lobular inflammation are scored as 0 (no foci), 1 (<2 foci per 200× field), 2 (2-4 foci per 200× field) and 3 (>4 foci per 200× field). Fibrosis is scored as stage F0 (no fibrosis), stage Fla (mild, zone 3, perisinusoidal fibrosis), stage Fib (moderate, zone 3, perisinusoidal fibrosis), stage F1c (portal/periportal fibrosis), stage F2 (perisinusoidal and portal/periportal fibrosis), stage F3 (bridging fibrosis), and stage F4 (cirrhosis). Diagnosis of NASH is based on accepted histological criteria. Severity of the disease is assessed using the NAS (NAFLD activity score) as the unweighted sum of scores of steatosis, hepatocyte ballooning, and lobular inflammation. Percentage of fibrosis is quantitated by morphometry from digitalised sirius red stained sections using the Aperio system after tuning the threshold of fibrosis detection under visual control. Results are expressed as collagen proportional area.

### Study of the Effects of Compounds on Non-Alcoholic Fatty Liver Disease (NAFLD) and Non-Alcoholic Steatohepatitis (NASH) in Methionine and Choline Deficient Mice

A study is performed to determine the effects of compounds of Formula (I-A), (II-A), (I), (II), or (III), or a pharmaceutically acceptable salt thereof, on non-alcoholic fatty liver disease (NAFLD) and non-alcoholic steatohepatitis (NASH) in male wildtype mice fed a methionine- and choline-deficient diet.

Wildtype mice housed in 12-hour light/dark cycles, with free access to food and water are used. At least 5 animals per time point are analyzed. All experiments are repeated at least three times. For dietary treatment, 8-12 weeks old male mice weighing 25 g are either fed a methionine- and choline-deficient diet (MCD to induce NASH) or chow diet (as a control). Animal experiments and evaluation of NAFLD and NASH as described above Examples for mice fed the high fat and high sucrose diet.

Unless otherwise defined, all technical and scientific terms used herein have the same meaning as commonly understood by one of ordinary skill in the art to which this disclosure belongs. In the specification, the singular forms also include the plural unless the context clearly dictates otherwise. Although methods and materials similar or equivalent to those described herein can be used in the practice of testing the present disclosure, suitable methods and materials are described below. All publications, patent applications, patents, and other references mentioned herein are hereby expressly incorporated by reference. The references cited herein are not admitted to be prior art of the claimed disclosure. In the case of conflict, the present specification, including definitions, will control. In addition, the materials, methods, and examples are illustrative only and are not intended to be limiting.

### EQUIVALENTS

Those skilled in the art will recognize, or be able to ascertain using no more than routine experimentation, many equivalents to the specific embodiments and methods described herein. Such equivalents are intended to be encompassed by the scope of the present disclosure.

1. A compound represented by Formula (I-A) or (II-A): 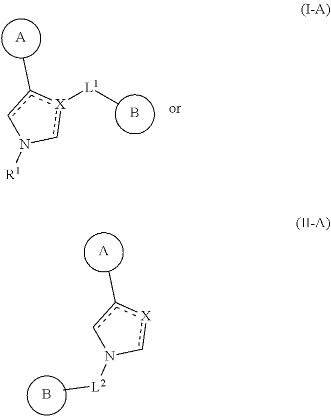
or a pharmaceutically acceptable salt and tautomer thereof, wherein:
each [symbol] independently represents a single bond or a double bond;
X is N, NH, C, CH, or CH₂;
R¹ is H, C₁₋₆alkyl, cycloalkyl, heterocyclyl, —C(O)R^{1a}, —CH₂-aryl, —CH₂-heteroaryl, aryl, or heteroaryl; wherein R^{1a} is C₁₋₆alkyl; and wherein —CH₂-aryl, —CH₂-heteroaryl, aryl, and heteroaryl are optionally substituted with C₁₋₆alkyl or halo;
A is alkyl, cycloalkyl, heterocyclyl, a fused bicyclic aryl, a fused bicyclic heteroaryl, —CH₂-aryl, —CH₂-heteroaryl, aryl, or heteroaryl; wherein the aryl or heteroaryl is optionally substituted with aryl, heteroaryl, —Y^{A}-aryl, or —Y^{A}-heteroaryl; wherein Y^{A} is —O—, —C(O)—, —N(R^{A1})—, S(O)—, or —S(O)₂—; wherein R^{A1} is H or C₁₋₆alkyl;
wherein the fused bicyclic aryl, the fused bicyclic heteroaryl, —CH₂-aryl, —CH₂— heteroaryl, each aryl, and each heteroaryl are optionally substituted with one or more substituents selected from the group consisting of alkyl, halo, haloalkyl, —CN, —N(R^{A})₂, —OH, and —O-alkyl; wherein each R^{A} is independently H or C₁₋₆alkyl;
L¹ is —C(O)—NR^{L1}—, —O—C(S)—NR^{L1}—, —O—C(O)—NR^{L1}—, —NR^{L1}—C(O)—, —NR^{L1}—C(O)—O—, —NH—C(O)—NH—, —NR^{L1}—C(S)—NR^{L1}—, —NR^{L1}—S(O)₂—, —S(O)₂—NR^{L1}—, —CH₂—CH₂—, —CH₂—NR^{L1}—, —NR^{L1}—CH₂—, —CH₂—O—, —O—CH₂—, —O—, —NH—, —C(O)-azetidinyl, —CH₂—NR^{L1}—C(O)—, —C(O)—NR^{L1}—CH₂—, or —C(O)—; wherein each R^{L1} is independently H or C₁₋₆alkyl; and
L² is —C(O)—NR^{L2}—, —S(O)₂—NR^{L2}—, —CH₂—CH₂—, —C(S)—NR^{L2}—, —C(O)—, or —S(O)₂—; wherein each R^{L2} is independently H or C₁₋₆alkyl; and
B is a fused bicyclic aryl, a fused bicyclic heteroaryl, —CH₂-aryl, —CH₂-heteroaryl, aryl, heteroaryl, cycloalkyl, —CH₂-heterocyclyl, or heterocyclyl, wherein the aryl, heteroaryl, cycloalkyl, or heterocyclyl is optionally substituted with aryl, heteroaryl, —Y^{B}-aryl, —Y^{B}— heteroaryl, —Y^{B}-heterocyclyl, or cycloalkyl; wherein Y^{B} is —O—, —CH₂—, —C(O)—, —N(R^{B1})—, —S(O)—, or —S(O)₂—; wherein R^{B1} is H or C₁₋₆alkyl;
wherein the fused bicyclic aryl, the fused bicyclic heteroaryl, —CH₂-aryl, —CH₂-heteroaryl, each aryl, each heteroaryl, each cycloalkyl, —CH₂-heterocyclyl, and each heterocyclyl are optionally substituted with one or more substituents selected from the group consisting of alkyl, halo, haloalkyl, —CN, —N(R^{B2})₂, —OH, —O-alkyl, and oxo;
wherein each R^{B2} is independently H or C₁₋₆alkyl;
wherein when the compound is Formula (I-A): A is optionally substituted phenyl or thiophenyl, and L¹ is —C(O)—NH—; then B is not 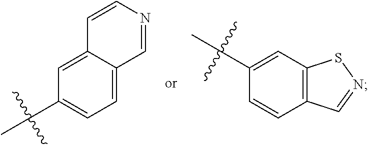 [SMILES: *c1ccc2cnccc2c1 ; *c1ccc2cnsc2c1]
wherein when the compound is Formula (I-A); A is phenyl, and L¹ is —C(O)—NH—; then B is not 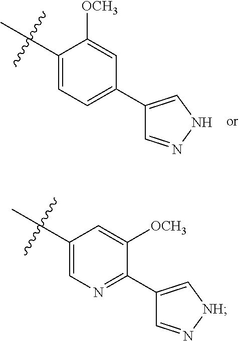 [SMILES: *c1ccc(-c2cn[nH]c2)cc1OC ; "or" ; *c1cnc(-c2cn[nH]c2)c(OC)c1]
wherein when the compound is Formula (I-A); A is a substituted phenyl and B is a substituted phenyl, then L¹ is not —C(O)—NH—, —NH—C(O)—, —NCH₃—C(O)—, or —NH—C(O)—NH—;
wherein when the compound is Formula (I-A); L¹ is —C(O)—NR^{L1}—CH₂— and B is an optionally substituted phenyl, substituted pyridyl, or 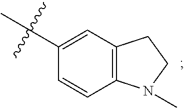 [SMILES: *c1ccc2c(c1)CCN2C]
then A is not substituted phenyl, substituted pyridyl, substituted thiophenyl, substituted thiazolyl, substituted pyrazolyl, 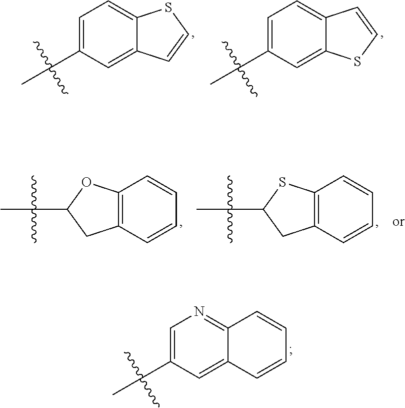 [SMILES: *c1ccc2sccc2c1 ; *c1ccc2ccsc2c1 ; *C1Cc2ccccc2O1 ; *C1Cc2ccccc2S1 ; *c1cnc2ccccc2c1]
wherein when the compound is Formula (I-A); B is optionally substituted —CH₂-aryl and A is optionally substituted aryl; then L¹ is not —C(O)—NH—;
wherein when the compound is Formula (II-A); A is optionally substituted phenyl and B is optionally substituted phenyl, then L¹ is not —C(O)—NCH₃—.

2-18. (canceled)

19. The compound of claim 1, or a pharmaceutically acceptable salt or tautomer thereof, wherein

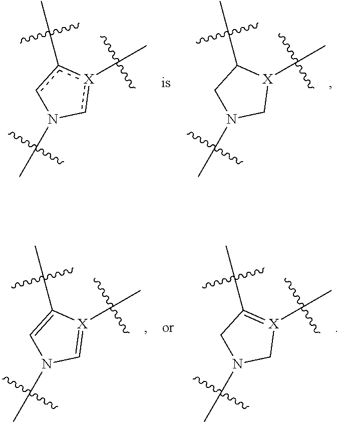

20-21. (canceled)

22. The compound of claim 1, or a pharmaceutically acceptable salt or tautomer thereof, wherein X is N or NH.

23. The compound of claim 1, or a pharmaceutically acceptable salt or tautomer thereof, wherein X is C, CH, or CH₂.

24. The compound of claim 1, or a pharmaceutically acceptable salt or tautomer thereof, wherein R¹ is H or C₁₋₆alkyl.

25. (canceled)

26. The compound of claim 1, or a pharmaceutically acceptable salt or tautomer thereof, wherein R¹ is cycloalkyl, heterocyclyl, —C(O)R^{1a}, or —CH₂-aryl.

27-29. (canceled)

30. The compound of claim 1, or a pharmaceutically acceptable salt thereof, wherein A is aryl.

31. The compound of claim 30, or a pharmaceutically acceptable salt or tautomer thereof, wherein the aryl is substituted with one or more substituents selected from the group consisting of alkyl, halo, —OH, and —O-alkyl.

32. The compound of claim 1, or a pharmaceutically acceptable salt or tautomer thereof, wherein A is 5- to 6-membered heteroaryl.

33. The compound of claim 32, or a pharmaceutically acceptable salt or tautomer thereof, wherein the heteroaryl is substituted with one or more substituents selected from the group consisting of alkyl, halo, —OH, and —O-alkyl.

34. The compound of claim 1, or a pharmaceutically acceptable salt or tautomer thereof, wherein A is alkyl, cycloalkyl, heterocyclyl, a fused bicyclic aryl, a fused bicyclic heteroaryl, —CH₂-aryl, or —CH₂-heteroaryl.

35-38. (canceled)

39. The compound of claim 1, or a pharmaceutically acceptable salt or tautomer thereof, wherein L¹ is —C(O)—NR^{L1}—.

40. The compound of claim 1, or a pharmaceutically acceptable salt or tautomer thereof, wherein L¹ is —O—C(S)—NR^{L1}—.

41. The compound of claim 1, or a pharmaceutically acceptable salt or tautomer thereof, wherein L¹ is —O—C(O)—NR^{L1}—.

42. The compound of claim 1, or a pharmaceutically acceptable salt or tautomer thereof, wherein L¹ is —NR^{L1}—C(S)—NR^{L1}—.

43. The compound of claim 1, or a pharmaceutically acceptable salt or tautomer thereof, wherein L¹ is —O—.

44. The compound of claim 1, or a pharmaceutically acceptable salt or tautomer thereof, wherein L¹ is —NR^{L1}—C(O)—, —NR^{L1}—C(O)—O—, —NH—C(O)—NH—, —NR^{L1}—S(O)₂— or —S(O)₂—NR^{L1}—.

45. The compound of claim 1, or a pharmaceutically acceptable salt or tautomer thereof, wherein L¹ is —CH₂—CH₂—, —CH₂—NR^{L1}—, —NR^{L1}—CH₂—, —CH₂—O—, —O—CH₂—, —NH—, or —C(O)-azetidinyl.

46. The compound of claim 1, or a pharmaceutically acceptable salt or tautomer thereof, wherein L² is —C(O)—NR^{L2}—.

47. The compound of claim 1, or a pharmaceutically acceptable salt or tautomer thereof, wherein L² is —S(O)₂—NR^{L2}— or —CH₂—CH₂—.

48-53. (canceled)

54. The compound of claim 1, or a pharmaceutically acceptable salt or tautomer thereof, wherein B is a fused bicyclic aryl or a fused bicyclic heteroaryl.

55. (canceled)

56. The compound of claim 1, or a pharmaceutically acceptable salt or tautomer thereof, wherein B is selected from the group consisting of

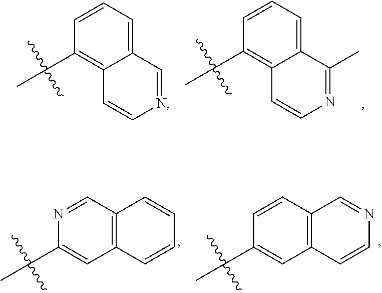 [SMILES: *c1cccc2cnccc12 ; *c1cccc2c(C)nccc12 ; *c1cc2ccccc2cn1 ; *c1ccc2cnccc2c1]

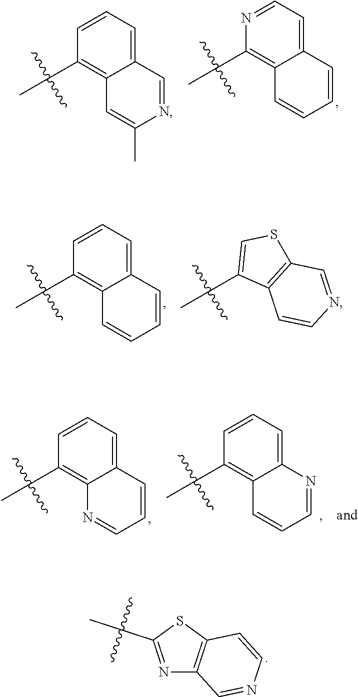

57. The compound of claim 1, or a pharmaceutically acceptable salt or tautomer thereof, wherein B is —CH₂-aryl or —CH₂-heteroaryl.

58. (canceled)

59. The compound of claim 1, or a pharmaceutically acceptable salt or tautomer thereof, wherein B is selected from the group consisting of

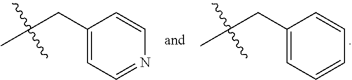 [SMILES: *Cc1ccncc1 ; *Cc1ccccc1]

60. The compound of claim 1, or a pharmaceutically acceptable salt or tautomer thereof, wherein B is aryl or heteroaryl.

61. The compound of claim 1, or a pharmaceutically acceptable salt or tautomer thereof, wherein B is aryl substituted with aryl or heteroaryl.

62. (canceled)

63. The compound of claim 1, or a pharmaceutically acceptable salt or tautomer thereof, wherein B is heteroaryl substituted with aryl or heteroaryl.

64. The compound of claim 1, or a pharmaceutically acceptable salt or tautomer thereof, wherein B is selected from the group consisting of

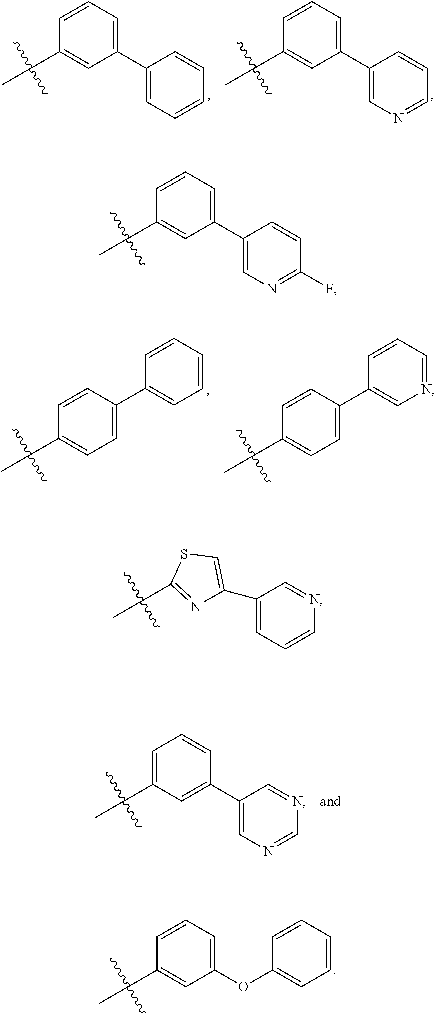

65. The compound of claim 1, or a pharmaceutically acceptable salt or tautomer thereof, wherein B is cycloalkyl.

66. The compound of claim 1, or a pharmaceutically acceptable salt or tautomer thereof, wherein B is cyclocyclyl cycloalkyl substituted with aryl, heteroaryl, —Y^{B}-aryl, —Y^{B}-heteroaryl.

67. The compound of claim 1, or a pharmaceutically acceptable salt or tautomer thereof, wherein B is —CH₂— heterocyclyl or heterocyclyl.

68. (canceled)

69. The compound of claim 1, or a pharmaceutically acceptable salt or tautomer thereof, wherein B is heterocyclyl substituted with aryl or heteroaryl.

70. The compound of claim 1, or a pharmaceutically acceptable salt or tautomer thereof, selected from the group consisting of

| Com- | |
|---|---|
| pound | |
| No. | Structure |
| I-1 | 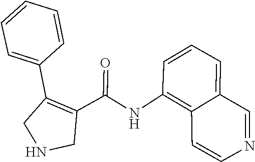 [SMILES: O=C(Nc1cccc2cnccc12)C1=C(c2ccccc2)CNC1] N-(isoquinolin-5-yl)-4-phenyl-2,5- dihydro-1H-pyrrole-3-carboxamide |
| I-2 | 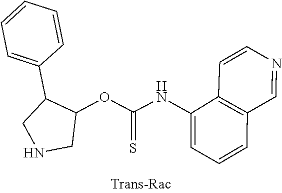 [SMILES: S=C(Nc1cccc2cnccc12)OC1CNCC1c1ccccc1] (±)-trans-O-(4-phenylpyrrolidin-3-yl) isoquinolin-5-ylcarbamothioate |
| I-3 | 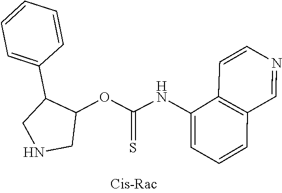 [SMILES: S=C(Nc1cccc2cnccc12)OC1CNCC1c1ccccc1] (±)-cis-O-(4-phenylpyrrolidin-3-yl) isoquinolin-5-ylcarbamothioate |
| I-4 | 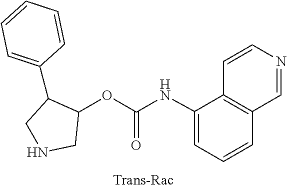 [SMILES: O=C(Nc1cccc2cnccc12)OC1CNCC1c1ccccc1] (±)-trans-4-phenylpyrrolidin-3-yl isoquinolin-5-ylcarbamate |
| I-5 | 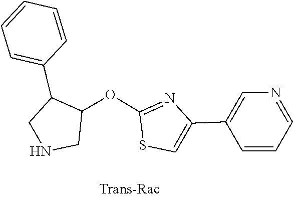 [SMILES: c1ccc(C2CNCC2Oc2nc(-c3cccnc3)cs2)cc1] (±)-trans-3-{2-[(4-phenylpyrrolidin-3- yl)oxy]-1,3-thiazol-4-yl}pyridine |
| I-6 | 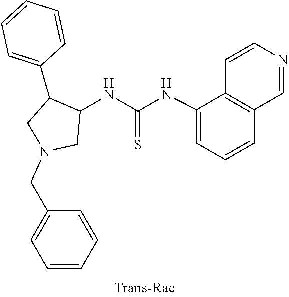 [SMILES: S=C(Nc1cccc2cnccc12)NC1CN(Cc2ccccc2)CC1c1ccccc1] (±)-trans-1-isoquinolin-5-yl-3-(1- benzyl-4-phenylpyrrolidin-3-yl)thioure |
| I-7 | 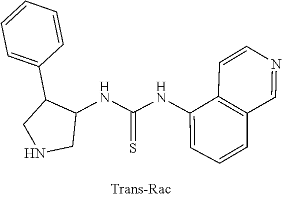 [SMILES: S=C(Nc1cccc2cnccc12)NC1CNCC1c1ccccc1] (±)-trans-1-isoquinolin-5-yl-3-(4- phenylpyrrolidin-3-yl)thiourea |
| I-8 | 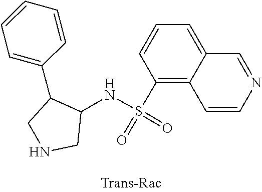 [SMILES: O=S(=O)(NC1CNCC1c1ccccc1)c1cccc2cnccc12] (±)-trans-N-(4-phenylpyrrolidin-3- yl)isoquinoline-5-sulfonamide |
| I-9 | 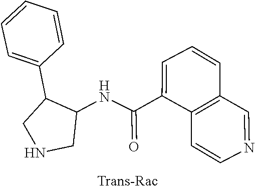 [SMILES: O=C(NC1CNCC1c1ccccc1)c1cccc2cnccc12] (±)-trans-N-(4-phenylpyrrolidin-3- yl)isoquinoline-5-carboxamide |
| I-10 | 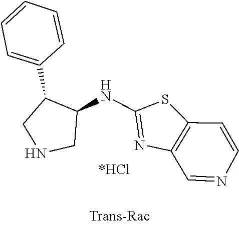 [SMILES: c1ccc([C@H]2CNC[C@@H]2Nc2nc3cnccc3s2)cc1] (±)-trans-N-[4-phenylpyrrolidin-3- yl][1,3]thiazolo[4,5-c]pyridin-2-amine hydrochloride |
| I-11 | 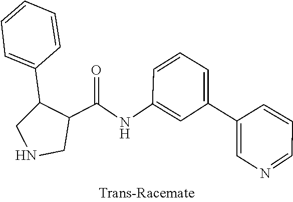 [SMILES: O=C(Nc1cccc(-c2cccnc2)c1)C1CNCC1c1ccccc1] (±)-trans-4-phenyl-N-[3-(pyridin-3- yl)phenyl]pyrrolidine-3-carboxamide |
| I-12 | 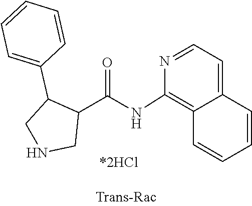 [SMILES: O=C(Nc1nccc2ccccc12)C1CNCC1c1ccccc1] (±)-trans-N-(isoquinolin-1-yl)-4- phenylpyrrolidine-3-carboxamide dihydrochloride |
| I-13 | 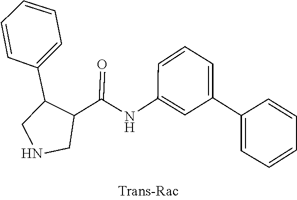 [SMILES: O=C(Nc1cccc(-c2ccccc2)c1)C1CNCC1c1ccccc1] (±)-trans-N-(biphenyl-3-yl)-4- phenylpyrrolidine-3-carboxamide |
| I-14 | 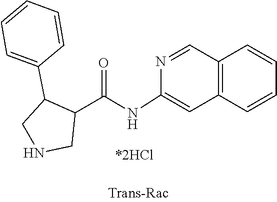 [SMILES: O=C(Nc1cc2ccccc2cn1)C1CNCC1c1ccccc1] (±)-trans-N-(isoquinolin-3-yl)-4- phenylpyrrolidine-3-carboxamide dihydrochloride |
| I-15 | 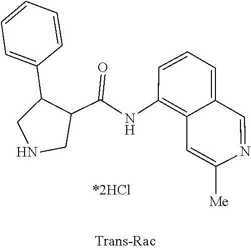 [SMILES: Cc1cc2c(NC(=O)C3CNCC3c3ccccc3)cccc2cn1] (±)-trans-N-(3-methylisoquinolin-5-yl)- 4-phenylpyrrolidine-3-caiboxamide dihydrochloride |
| I-16 | 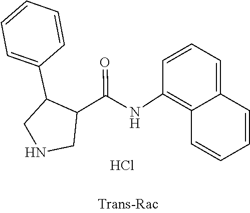 [SMILES: O=C(Nc1cccc2ccccc12)C1CNCC1c1ccccc1 ; Cl] (±)-trans-N-(naphthalen-1-yl)-4- phenylpyrrolidine-3-carboxamide hydrochloride |
| I-17 | 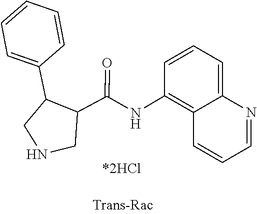 [SMILES: O=C(Nc1cccc2ncccc12)C1CNCC1c1ccccc1] (±)-trans-4-phenyl-N-(quinolin-5- yl)pyrrolidine-3-carboxamide dihydrochloride |
| I-18 | 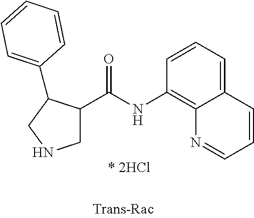 [SMILES: O=C(Nc1cccc2cccnc12)C1CNCC1c1ccccc1] (±)-trans-4-phenyl-N-(quinolin-8- yl)pyrrolidine-3-carboxamide dihydrochloride |
| I-19 | 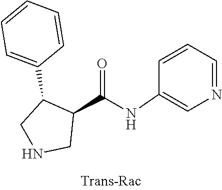 [SMILES: O=C(Nc1cccnc1)[C@H]1CNC[C@@H]1c1ccccc1] (±)-trans-4-phenyl-N-(pyridin-3- yl)pyrrolidine-3-carboxamide dihydrochloride |
| I-20 | 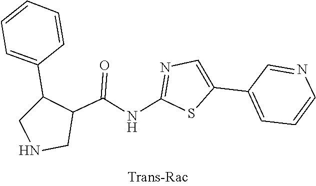 [SMILES: O=C(Nc1ncc(-c2cccnc2)s1)C1CNCC1c1ccccc1] (±)-trans-4-phenyl-N-[5-[5-(pyridin-3-yl)- 1,3-thiazol-2-yl]pyrrolidine-3- carboxamide |
| I-21 | 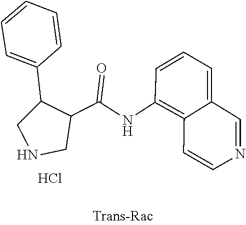 [SMILES: O=C(Nc1cccc2cnccc12)C1CNCC1c1ccccc1 ; Cl] (±)-trans-N-(isoquinolin-5-yl)-4- phenylpyrrolidine-3-carboxamide dihydrochloride |
| I-22 | 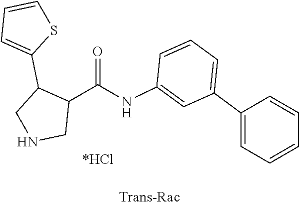 [SMILES: O=C(Nc1cccc(-c2ccccc2)c1)C1CNCC1c1cccs1] (±)-trans-N-(biphenyl-3-yl)-4-(thiophen- 2-yl)pyrrolidine-3-carboxamide hydrochloride |
| I-23 | 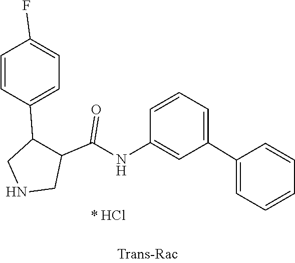 [SMILES: O=C(Nc1cccc(-c2ccccc2)c1)C1CNCC1c1ccc(F)cc1] (±)-trans-N-(biphenyl-3-yl)-4-(4- fluorophenyl)pyrrolidine-3-carboxamide hydrochloride |
| I-24 | 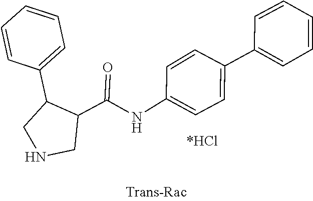 [SMILES: O=C(Nc1ccc(-c2ccccc2)cc1)C1CNCC1c1ccccc1] (±)-trans-N-(biphenyl-4-yl)-4- phenylpyrrolidine-3-carboxamide hydrochloride |
| I-25 | 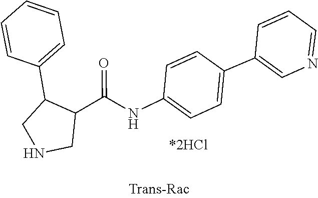 [SMILES: O=C(Nc1ccc(-c2cccnc2)cc1)C1CNCC1c1ccccc1] (±)-trans-4-phenyl-N-[4-(pyridin-3- yl)phenyl]pyrrolidine-3-carboxamide dihydrochloride |
| I-26 | 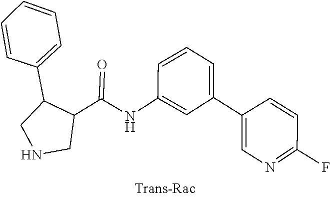 [SMILES: O=C(Nc1cccc(-c2ccc(F)nc2)c1)C1CNCC1c1ccccc1] (±)-trans-N-[3-(6-fluoropyridin-3- yl)phenyl]-4-phenylpyrrolidine-3- carboxamide |
| I-27 | 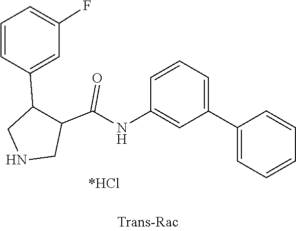 [SMILES: O=C(Nc1cccc(-c2ccccc2)c1)C1CNCC1c1cccc(F)c1] (±)-trans-N-(biphenyl-3-yl)-4-(3- fluorophenyl)pyrrolidine-3-carboxamide hydrochloride |
| I-28 | 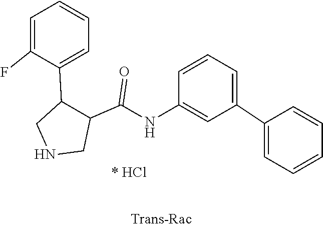 [SMILES: O=C(Nc1cccc(-c2ccccc2)c1)C1CNCC1c1ccccc1F] (±)-trans-N-(biphenyl-3-yl)-4-(2- fluorophenyl)pyrrolidine-3-carboxamide hydrochloride |
| I-29 | 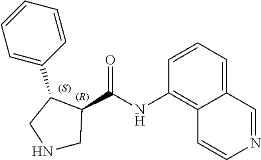 (3R,4S)-N-(isoquinolin-5-yl)-4- phenylpyrrolidine-3-carboxamide |
| I-30 | 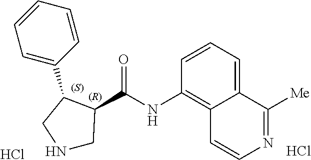 (3R,4S)-N-(1-methylisoquinolin-5-yl)-4- phenylpyrrolidine-3-carboxamide dihydrochloride |
| I-31 | 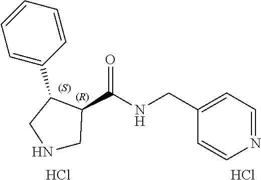 (3R,4S)-4-phenyl-N-(pyridin-4- ylmethyl)pyrrolidine-3-carboxamide dihydrochloride |
| I-32 | 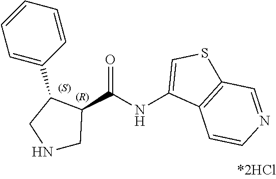 (3R,4S)-4-phenyl-N-(thieno[2,3- c]pyridin-3-yl)pyrrolidine-3- carboxamide dihydrochloride |
| I-33 | 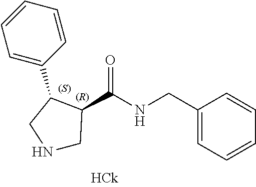 (3R,4S)-N-benzyl-4-phenylpyrrolidine- 3-carboxamide hydrochloride |
| I-34 | 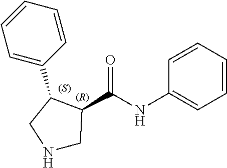 (3R,4S)-N,4-diphenylpyrrolidine-3- carboxamide |
| I-35 | 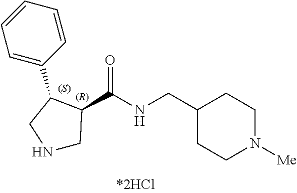 (3R,4S)-N-[(1-methylpiperidin-4- yl)methyl]-4-phenylpyrrolidine-3- carboxamide dihydrochloride |
| I-36 | 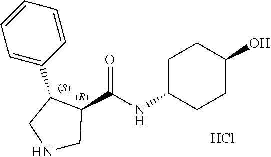 (3R,4S)-N-[(1,4-trans)-4- hydroxycyclohexyl]-4- phenylpyrrolidine-3-carboxamide hydrochloride |
| I-37 | 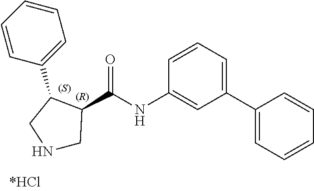 (3R,4S)-N-(biphenyl-3-yl)-4- phenylpyrrolidine-3-carboxamide hydrochloride |
| I-38 | 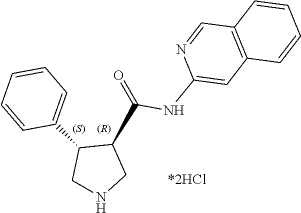 (3R,4S)-N-(isoquinolin-3-yl)-4- phenylpyrrolidine-3-carboxamide dihydrochloride |
| I-39 | 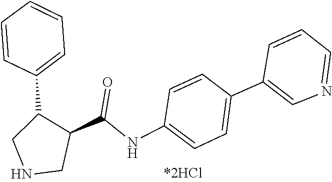 (3R,4S)-4-phenyl-N-[4-(pyridin-3- yl)phenyl]pyrrolidine-3-carboxamide dihydrochloride |
| I-40 | 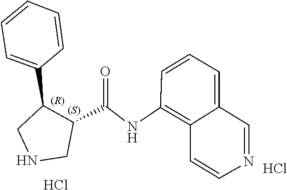 (3S,4R)-N-(isoquinolin-5-yl)-4- phenylpyrrolidine-3-carboxamide dihydrochloride |
| I-41 | 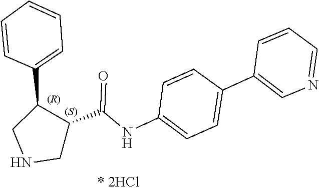 (3S,4R)-4-phenyl-N-[4-(pyridin-3- yl)phenyl]pyrrolidine-3-carboxamide dihydrochloride |
| I-42 | 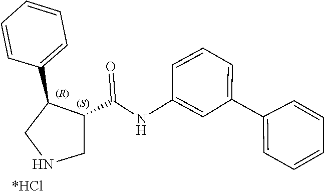 (3S,4R)-N-(biphenyl-3-yl)-4- phenylpyrrolidine-3-carboxamide hydrochloride |
| I-43 | 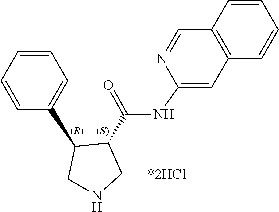 (3S,4R)-N-(isoquinolin-3-yl)-4- phenylpyrrolidine-3-carboxamide dihydrochloride |
| I-44 | 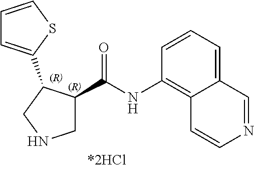 (3R,4R)-N-(isoquinolin-5-yl)-4- (thiophen-2-yl)pyrrolidine-3- carboxamide dihydrochloride |
| I-45 | 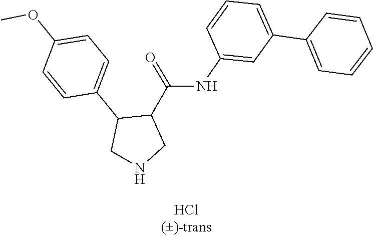 [SMILES: COc1ccc(C2CNCC2C(=O)Nc2cccc(-c3ccccc3)c2)cc1] (±)-trans-N-(biphenyl-3-yl)-4-(4- methoxyphenyl)pyrrolidine-3- carboxamide hydrochloride |
| I-46 | 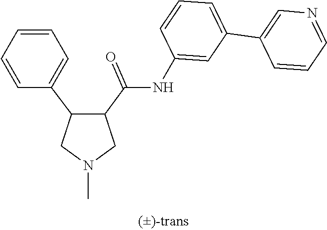 [SMILES: CN1CC(C(=O)Nc2cccc(-c3cccnc3)c2)C(c2ccccc2)C1] (±)-trans-1-methyl-4-phenyl-N-[3- (pyridin-3-yl)phenyl]pyrrolidine-3- carboxamide |
| I-47 | 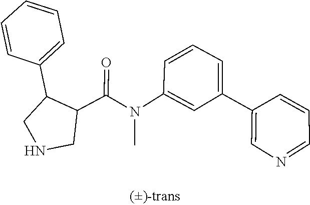 [SMILES: CN(C(=O)C1CNCC1c1ccccc1)c1cccc(-c2cccnc2)c1] (±)-trans-N-methyl-4-phenyl-N-[3- (pyridin-3-yl)phenyl]pyrrolidine-3- carboxamide |
| I-48 | 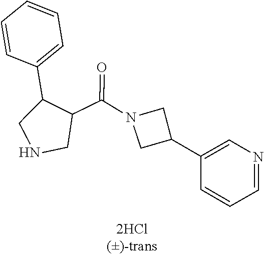 [SMILES: O=C(C1CNCC1c1ccccc1)N1CC(c2cccnc2)C1] (±)-trans-(4-phenylpyrrolidin-3-yl)[3- (pyridin-3-yl)azetidin-1-yl]methanone dihydrochloride |
| I-49 | 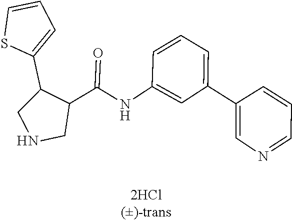 [SMILES: O=C(Nc1cccc(-c2cccnc2)c1)C1CNCC1c1cccs1] (±)-trans-(4-phenylpyrrolidin-3-yl)[3- (pyridin-3-yl)azetidin-1-yl]methanone dihydrochloride |
| I-50 | 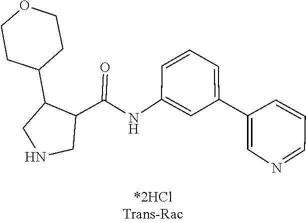 [SMILES: O=C(Nc1cccc(-c2cccnc2)c1)C1CNCC1C1CCOCC1] (±)-trans-N-[3-(pyridin-3-yl)phenyl]-4- (tetrahydro-2H-pyran-4-yl)pyrrolidine- 3-carboxamide dihydrochloride |
| I-51 | 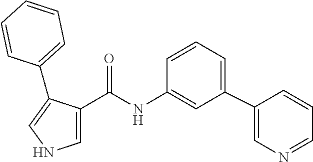 [SMILES: O=C(Nc1cccc(-c2cccnc2)c1)c1c[nH]cc1-c1ccccc1] 4-phenyl-N-[3-(pyridin-3-yl)phenyl]- 1H-pyrrole-3-carboxamide |
| I-52 | 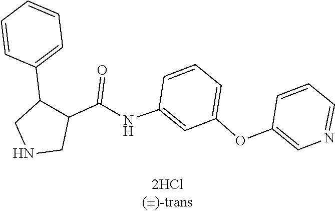 [SMILES: O=C(Nc1cccc(Oc2cccnc2)c1)C1CNCC1c1ccccc1] (±)-trans-N-(3-phenoxyphenyl)-4- phenylpyrrolidine-3-carboxamide dihydrochloride |
| I-53 | 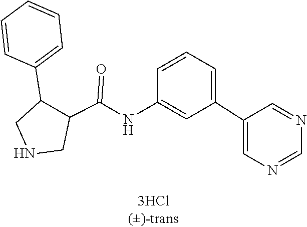 [SMILES: O=C(Nc1cccc(-c2cncnc2)c1)C1CNCC1c1ccccc1] (±)-trans-4-phenyl-N-[3-(pyrimidin-5- yl)phenyl]pyrrolidine-3-carboxamide triihydrochloride |
| I-54 | 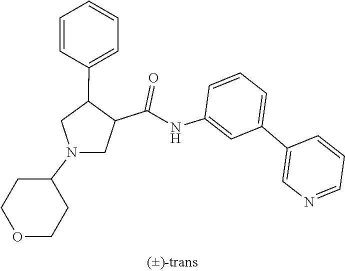 [SMILES: O=C(Nc1cccc(-c2cccnc2)c1)C1CN(C2CCOCC2)CC1c1ccccc1] (±)-trans-4-phenyl-N-[3-(pyridin-3- yl)phenyl]-1-(tetrahydro-2H-pyran-4- yl)pyrrolidine-3-carboxamide |
| I-55 | 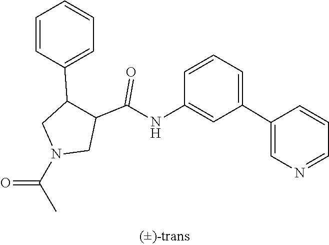 [SMILES: CC(=O)N1CC(C(=O)Nc2cccc(-c3cccnc3)c2)C(c2ccccc2)C1] (±)-trans-1-acetyl-4-phenyl-N-[3- (pyridin-3-yl)phenyl]pyrrolidine-3- carboxamide |
| I-56 | 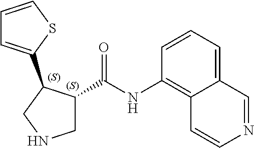 (3S,4S)-N-(isoquinolin-5-yl)-4- (thiophen-2-yl)pyrrolidine-3- carboxamide |
| I-57 | 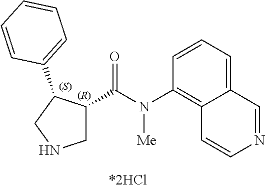 (3R,4S)-N-(isoquinolin-5-yl)-N-methyl- 4-phenylpyrrolidine-3-cathoxamide dihydrochloride |
| I-58 | 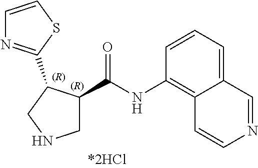 (3R,4R)-N-(isoquinolin-5-yl)-4-(1,3- thiazol-2-yl)pyrrolidine-3-carboxamide dihydrochloride |
| I-59 | 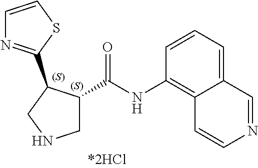 (3S,4S)-N-(isoquinolin-5-yl)-4-(1,3- thiazol-2-yl)pyrrolidine-3-carboxamide dihydrochloride |

71. The compound of claim 1, or a pharmaceutically acceptable salt or tautomer thereof, selected from the group consisting of

| Com- | |
|---|---|
| pound | |
| No. | Structure |
| I-60 | 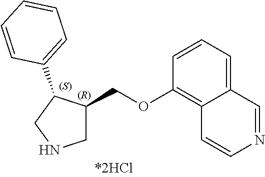 |
| I-61 | 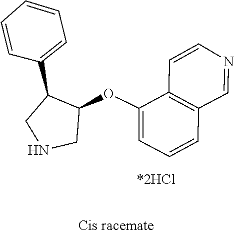 [SMILES: c1ccc([C@@H]2CNC[C@@H]2Oc2cccc3cnccc23)cc1] |
| I-62 | 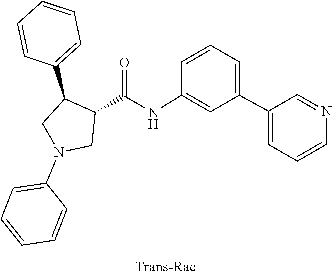 [SMILES: O=C(Nc1cccc(-c2cccnc2)c1)[C@@H]1CN(c2ccccc2)C[C@H]1c1ccccc1] |
| I-63 | 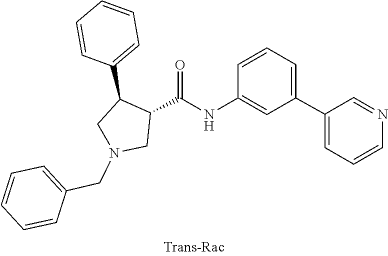 [SMILES: O=C(Nc1cccc(-c2cccnc2)c1)[C@@H]1CN(Cc2ccccc2)C[C@H]1c1ccccc1] |
| I-64 | 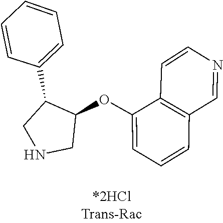 [SMILES: c1ccc([C@H]2CNC[C@@H]2Oc2cccc3cnccc23)cc1] |
| I-65 | 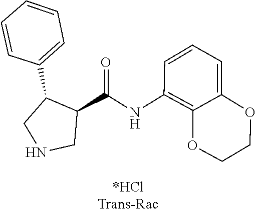 [SMILES: O=C(Nc1cccc2c1OCCO2)[C@H]1CNC[C@@H]1c1ccccc1] |
| I-66 | 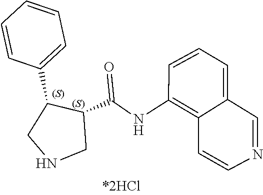 |
| I-67 | 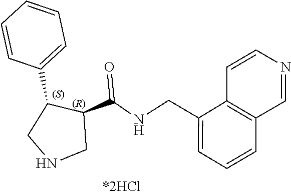 |
| I-68 | 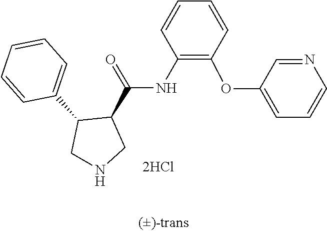 [SMILES: O=C(Nc1ccccc1Oc1cccnc1)[C@H]1CNC[C@@H]1c1ccccc1] |
| I-69 | 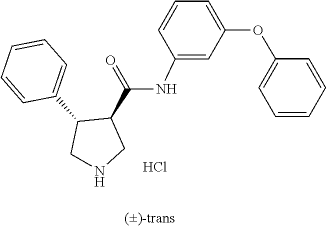 [SMILES: O=C(Nc1cccc(Oc2ccccc2)c1)[C@H]1CNC[C@@H]1c1ccccc1 ; Cl] |
| I-70 | 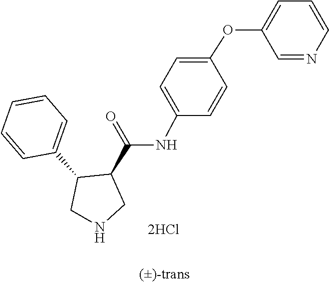 [SMILES: O=C(Nc1ccc(Oc2cccnc2)cc1)[C@H]1CNC[C@@H]1c1ccccc1] |
| I-71 | 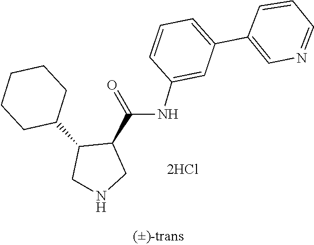 [SMILES: O=C(Nc1cccc(-c2cccnc2)c1)[C@H]1CNC[C@@H]1C1CCCCC1] |
| I-72 | 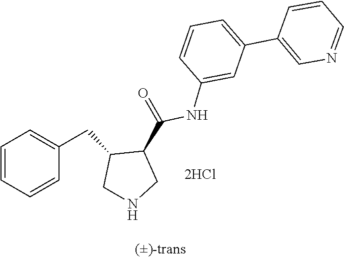 [SMILES: O=C(Nc1cccc(-c2cccnc2)c1)[C@H]1CNC[C@@H]1Cc1ccccc1] |
| I-73 | 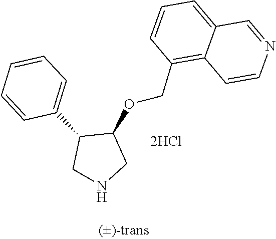 [SMILES: c1ccc([C@H]2CNC[C@@H]2OCc2cccc3cnccc23)cc1] |
| I-74 | 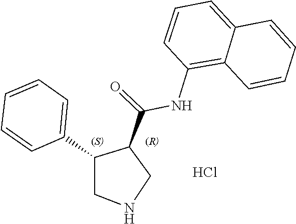 |
| I-75 | 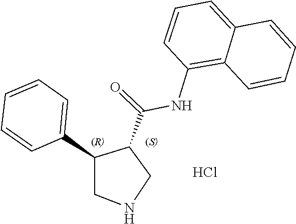 |
| I-76 | 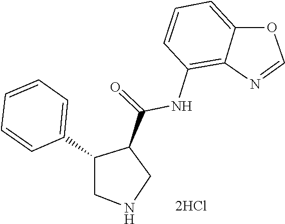 [SMILES: O=C(Nc1cccc2ocnc12)[C@H]1CNC[C@@H]1c1ccccc1 ; "2HCl"] |

72. The compound of claim 1, or a pharmaceutically acceptable salt or tautomer thereof, selected from the group consisting of

| Compound | |
|---|---|
| No. | Structure |
| I-77 | 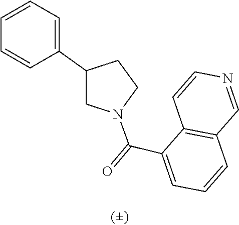 [SMILES: O=C(c1cccc2cnccc12)N1CCC(c2ccccc2)C1] |
| I-78 | 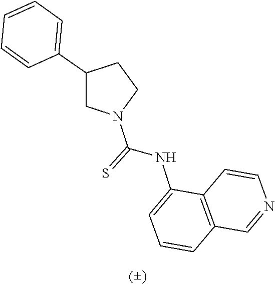 [SMILES: S=C(Nc1cccc2cnccc12)N1CCC(c2ccccc2)C1] |
| I-79 | 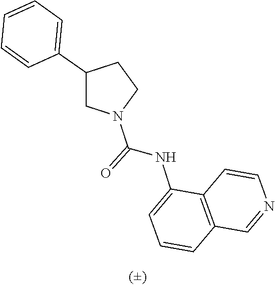 [SMILES: O=C(Nc1cccc2cnccc12)N1CCC(c2ccccc2)C1] |
| I-80 | 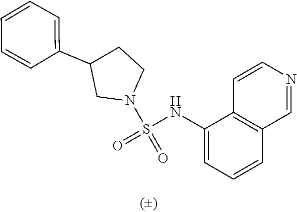 [SMILES: O=S(=O)(Nc1cccc2cnccc12)N1CCC(c2ccccc2)C1] |

73. The compound of claim 1, or a pharmaceutically acceptable salt or tautomer thereof, selected from the group consisting of

| Compound | |
|---|---|
| No. | Structure |
| I-81 | 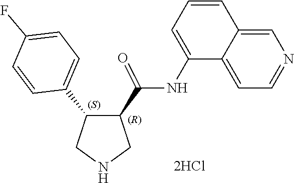 |
| I-82 | 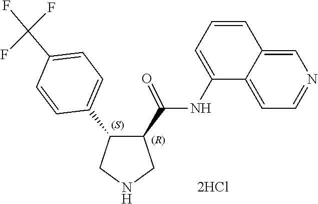 |
| I-83 | 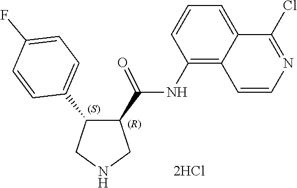 |
| I-84 | 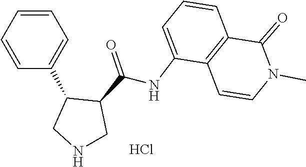 [SMILES: Cn1ccc2c(NC(=O)[C@H]3CNC[C@@H]3c3ccccc3)cccc2c1=O ; Cl] |
| I-85 | 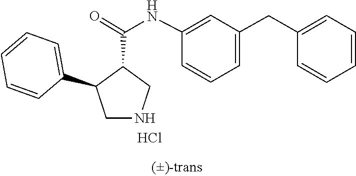 [SMILES: O=C(Nc1cccc(Cc2ccccc2)c1)[C@@H]1CNC[C@H]1c1ccccc1 ; Cl] |
| I-86 | 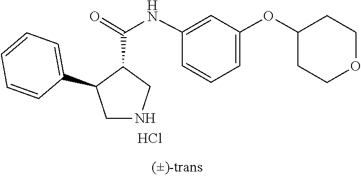 [SMILES: O=C(Nc1cccc(OC2CCOCC2)c1)[C@@H]1CNC[C@H]1c1ccccc1 ; Cl] |
| I-87 | 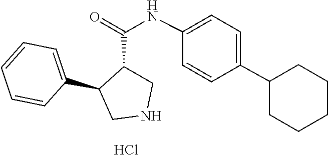 [SMILES: O=C(Nc1ccc(C2CCCCC2)cc1)[C@@H]1CNC[C@H]1c1ccccc1 ; Cl] |
| I-88 | 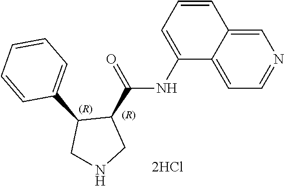 |
| I-89 | 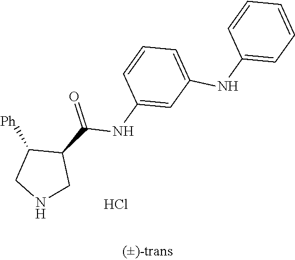 [SMILES: O=C(Nc1cccc(Nc2ccccc2)c1)[C@H]1CNC[C@@H]1c1ccccc1 ; Cl] |
| I-90 | 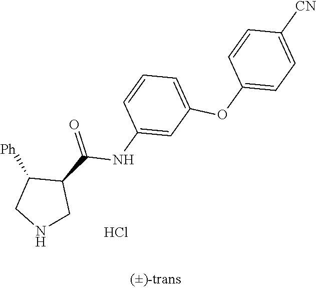 [SMILES: N#Cc1ccc(Oc2cccc(NC(=O)[C@H]3CNC[C@@H]3c3ccccc3)c2)cc1 ; Cl] |
| I-91 | 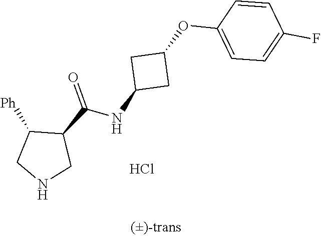 [SMILES: O=C(N[C@H]1C[C@H](Oc2ccc(F)cc2)C1)[C@H]1CNC[C@@H]1c1ccccc1 ; Cl] |
| I-92 | 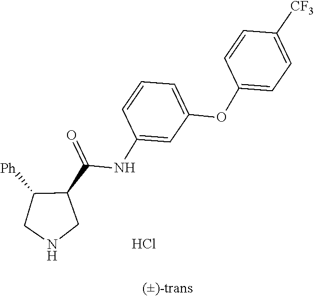 [SMILES: O=C(Nc1cccc(Oc2ccc(C(F)(F)F)cc2)c1)[C@H]1CNC[C@@H]1c1ccccc1 ; Cl] |
| I-93 | 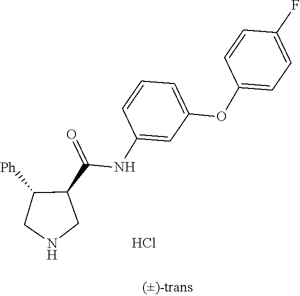 [SMILES: O=C(Nc1cccc(Oc2ccc(F)cc2)c1)[C@H]1CNC[C@@H]1c1ccccc1 ; Cl] |
| I-94 | 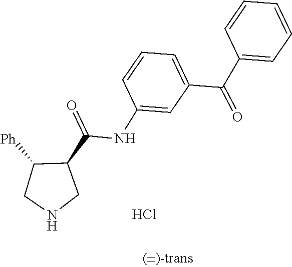 [SMILES: O=C(c1ccccc1)c1cccc(NC(=O)[C@H]2CNC[C@@H]2c2ccccc2)c1 ; Cl] |
| I-95 | 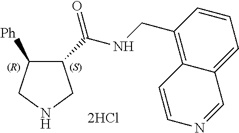 |
| I-96 | 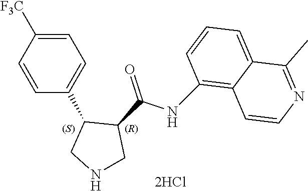 |
| I-97 | 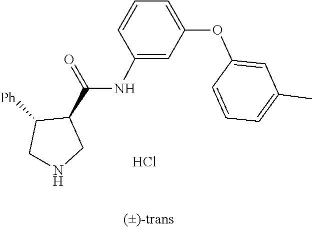 [SMILES: Cc1cccc(Oc2cccc(NC(=O)[C@H]3CNC[C@@H]3c3ccccc3)c2)c1 ; Cl] |
| I-98 | 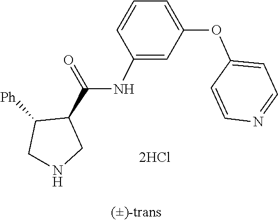 [SMILES: O=C(Nc1cccc(Oc2ccncc2)c1)[C@H]1CNC[C@@H]1c1ccccc1] |
| I-99 | 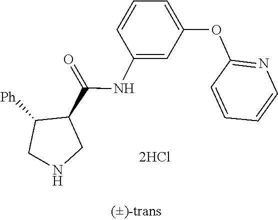 [SMILES: O=C(Nc1cccc(Oc2ccccn2)c1)[C@H]1CNC[C@@H]1c1ccccc1] |
| I-100 | 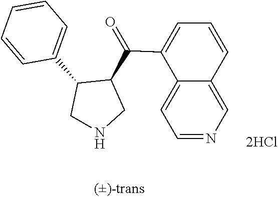 [SMILES: O=C(c1cccc2cnccc12)[C@H]1CNC[C@@H]1c1ccccc1] |
| I-101 | 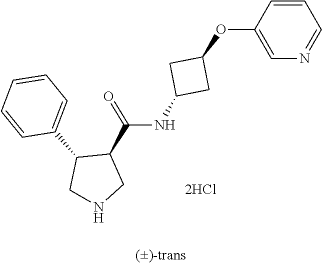 [SMILES: O=C(N[C@H]1C[C@H](Oc2cccnc2)C1)[C@H]1CNC[C@@H]1c1ccccc1] |
| I-102 | 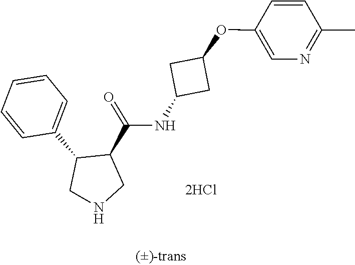 [SMILES: Cc1ccc(O[C@H]2C[C@H](NC(=O)[C@H]3CNC[C@@H]3c3ccccc3)C2)cn1] |
| I-103 | 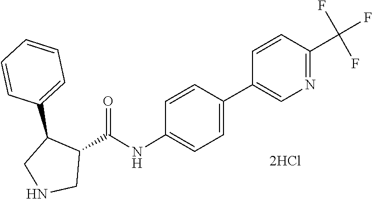 [SMILES: O=C(Nc1ccc(-c2ccc(C(F)(F)F)nc2)cc1)[C@@H]1CNC[C@H]1c1ccccc1] |
| I-104 | 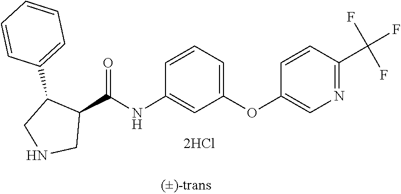 [SMILES: O=C(Nc1cccc(Oc2ccc(C(F)(F)F)nc2)c1)[C@H]1CNC[C@@H]1c1ccccc1] |
| I-105 | 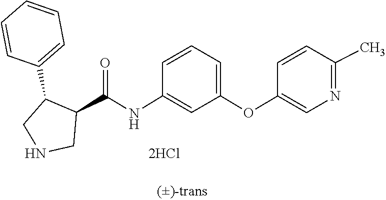 [SMILES: Cc1ccc(Oc2cccc(NC(=O)[C@H]3CNC[C@@H]3c3ccccc3)c2)cn1] |
| I-106 | 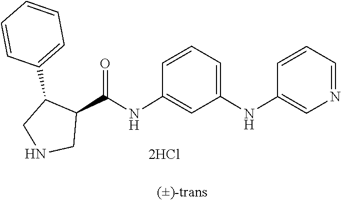 [SMILES: O=C(Nc1cccc(Nc2cccnc2)c1)[C@H]1CNC[C@@H]1c1ccccc1] |
| I-107 | 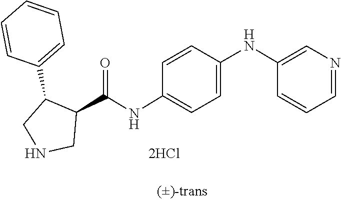 [SMILES: O=C(Nc1ccc(Nc2cccnc2)cc1)[C@H]1CNC[C@@H]1c1ccccc1] |
| I-108 | 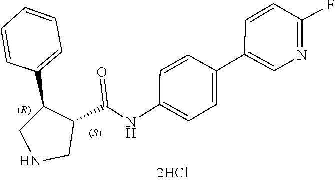 |
| I-109 | 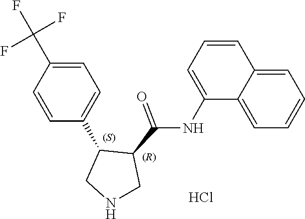 |
| I-110 | 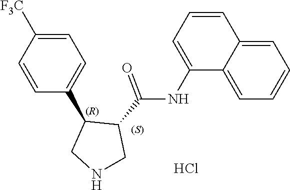 |
| I-111 | 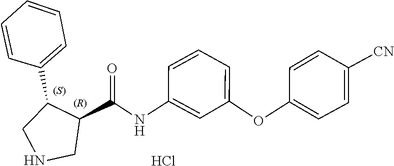 |
| I-112 | 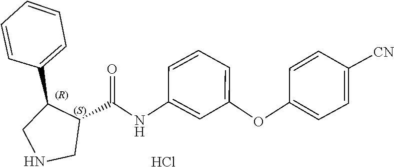 |
| I-113 | 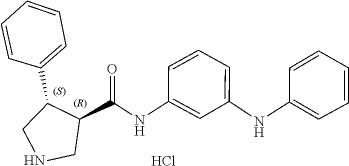 |
| I-114 | 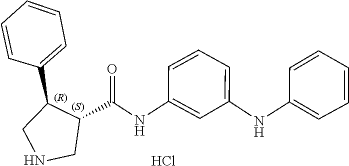 |
| I-115 | 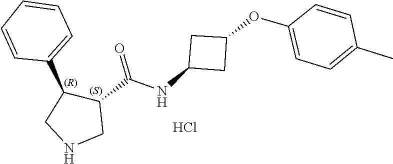 |
| I-116 | 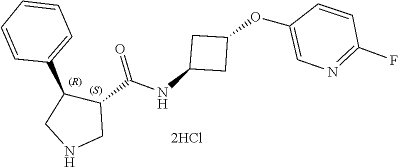 |
| I-117 | 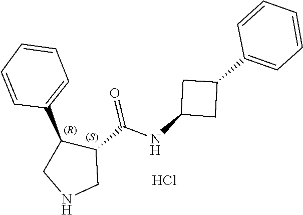 |
| I-118 | 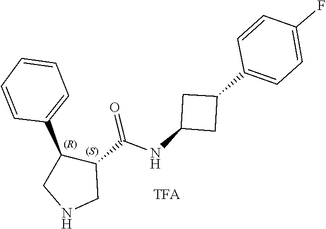 |
| I-119 | 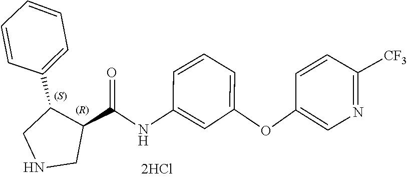 |
| I-120 | 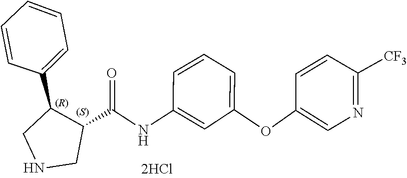 |
| I-121 | 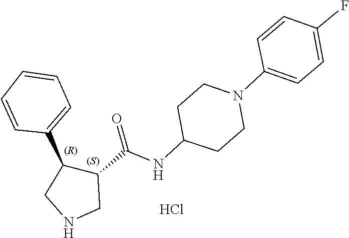 |
| I-122 | 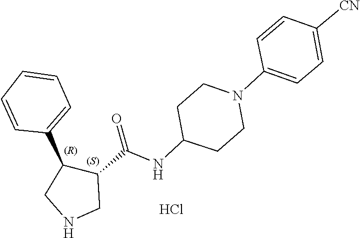 |
| I-123 | 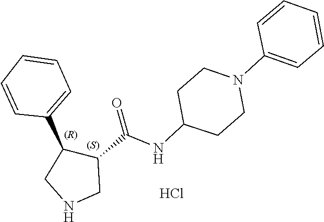 |

74. A pharmaceutical composition, comprising the compound of claim 1, or a pharmaceutically acceptable salt or tautomer thereof, and a pharmaceutically acceptable excipient.

75-77. (canceled)

78. A method of treating or reducing the effect of a disease or disorder associated with NR2F6 modulation, the method comprising administration of an effective amount of a compound of claim 1, or a pharmaceutically acceptable salt or tautomer thereof.

79. The method of claim 78, wherein the disease or disorder comprises an augmented autoimmune response or the disorder is cancer, haematological malignancy, gastrointestinal disease or disorder, or a condition associated with hepatic steatosis.

80-94. (canceled)
